(19) United States
(12) Patent Application Publication
(10) Pub. No.: US 2024/0298530 A1
(43) Pub. Date: Sep. 5, 2024

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Doo-Hyeon MOON, Gyeonggi-do (KR); Mi-Ja LEE, Gyeonggi-do (KR); Yea-Mi SONG, Gyeonggi-do (KR); Hyun-Ju KANG, Gyeonggi-do (KR); Hyo-Jung LEE, Gyeonggi-do (KR); Kyoung-Jin PARK, Gyeonggi-do (KR)

(21) Appl. No.: 18/417,068

(22) Filed: Jan. 19, 2024

(30) Foreign Application Priority Data
Feb. 2, 2023 (KR) 10-2023-0014029

(51) Int. Cl. H10K 85/60; C07D 403/04; C07D 403/10; C09K 11/06; H10K 50/11; H10K 101/00

(52) U.S. Cl. CPC H10K 85/654; C07D 403/04; C07D 403/10; C09K 11/06; H10K 85/624; H10K 85/6572; C09K 2211/1018; H10K 50/11; H10K 2101/90

(57) ABSTRACT

The present disclosure relates to a plurality of host materials, an organic electroluminescent compound, and an organic electroluminescent device comprising the same. By comprising the specific combination of the compounds according to the present disclosure as a plurality of host materials, or by comprising the compound according to the present disclosure, an organic electroluminescent device with low driving voltage and/or long lifespan characteristics can be provided, compared to a conventional organic electroluminescent device.

### TECHNICAL FIELD

The present disclosure relates to a plurality of host materials, an organic electroluminescent compound, and an organic electroluminescent device comprising the same.

### BACKGROUND ART

The TPD/Alq₃ bilayer small molecule organic electroluminescent device (OLED) with green-emission, which is constituted with a light-emitting layer and a charge transport layer, was first developed by Tang, et al., of Eastman Kodak in 1987. Thereafter, the studies on an OLED have been rapidly affected and OLEDs commercialized. At present, an organic electroluminescent device mainly includes phosphorescent materials having excellent luminous efficiency in panel realization. Accordingly, for prolonged use and high resolution of the display, an OLED having high luminous efficiency and/or long lifespan is necessary.

Various materials or concepts have been proposed for the organic layer of an organic electroluminescent device in order to improve luminous efficiency, driving voltage and/or lifespan, but they have not been satisfactory for practical use. Accordingly, there is a continuous need to develop organic electroluminescent devices with improved performance, such as improved driving voltage, luminous efficiency, power efficiency, and/or lifespan characteristics, compared to the previously disclosed organic electroluminescent devices.

Chinese Patent Application Laid-open No. 109791982 and Korean Patent Application Laid-open No. 10-2018-0080978 do not specifically disclose a plurality of host materials comprising a specific combination of compounds as described in the present disclosure. In addition, there is still a need to develop light-emitting materials with improved performance, such as improved driving voltage, luminous efficiency, and/or lifespan characteristics, compared to the previously disclosed organic electroluminescent devices.

### DISCLOSURE OF THE INVENTION

### Problems to be Solved

The object of the present disclosure is firstly, to provide a plurality of host materials which is able to produce an organic electroluminescent device with improved driving voltage and/or lifespan characteristics, and secondly, to provide an organic electroluminescent compound with a new structure suitable for application to organic electroluminescent devices. The other object of the present disclosure is to provide an organic electroluminescent device with low driving voltage and/or long lifespan characteristics by comprising a specific combination of compounds or an organic electroluminescent compound according to the present disclosure.

### Solution to Problems

As a result of intensive studies to solve the technical problem above, the present inventors found that the aforementioned objective can be achieved by a plurality of host materials comprising at least one first host compound represented by the following Formula 1 and at least one second host compound represented by the following Formula 2 wherein at least one of the first host compound and the second host compound includes at least one deuterium, so that the present invention was completed.

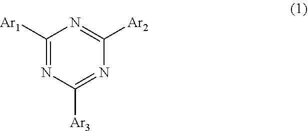

In Formula 1,
Ar₁ to Ar₃ each independently represent, a substituted or unsubstituted (C6-C30)aryl or a substituted or unsubstituted (3- to 30-membered)heteroaryl; provided that at least one of Ar₁ to Ar₃ is a carbazole group represented by the following Formula 1-1;

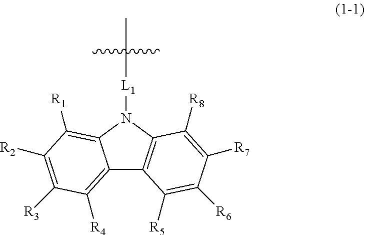

In Formula 1-1,
L₁ represents a single bond or (C6-C30)arylene unsubstituted or substituted with deuterium;
R₁ to R₈ each independently represent, hydrogen, deuterium, or (C6-C30)aryl unsubstituted or substituted with deuterium, (C1-C30)alkyl, (C6-C30)aryl, or a combination thereof; provided that at least one of R₁ to R₄ is represented by the following formulas 1-11 or 1-12;

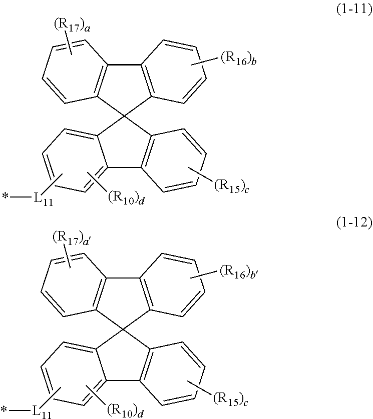

In formulas 1-11 and 1-12,
L₁₁ represents a single bond or (C6-C30)arylene unsubstituted or substituted with deuterium; R₁₀ and R₁₅ to R₁₇ each independently represent, hydrogen, deuterium, or (C6-C30)aryl unsubstituted or substituted with deuterium;
a to c each independently represent, an integer of 1 to 4; d is an integer of 1 to 3; and a′ and b′ each independently represent, an integer of 1 to 5; when a to d, a′, and b′ are 2 or more, each of R₁₀ and each of R₁₅ to each of R₇ may be the same or different; and
* represents a linking site with R₁ to R₄;

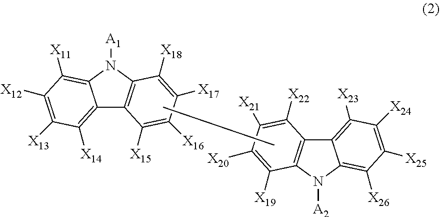

In Formula 2,
A₁ and A₂ each independently represent, (C6-C30)aryl unsubstituted or substituted with deuterium, dibenzofuranyl unsubstituted or substituted with deuterium, dibenzothiophenyl unsubstituted or substituted with deuterium, or carbazolyl unsubstituted or substituted with deuterium;
X₁₁ to X₂₆ each independently represent hydrogen, deuterium, a substituted or unsubstituted (C6-C30)aryl, or a substituted or unsubstituted (3- to 30-membered)heteroaryl; or may be linked to the adjacent substituents to form a ring(s); and
any one of X₁₅ to X₁₈ is linked to any one of X₁₉ to X₂₂.

### Advantageous Effects of Invention

By comprising a plurality of host materials and/or an organic electroluminescent compound according to the present disclosure, an organic electroluminescent device having low driving voltage and/or long lifespan characteristics can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials comprising at least one first host compound represented by Formula 1 and at least one a second host compound represented by Formula 2 where at least one of the first host compound and the second host compound includes at least one deuterium, and an organic electroluminescent device comprising the same.

The present disclosure relates to an organic electroluminescent compound represented by Formula 1′, an organic electroluminescent material comprising the organic electroluminescent compound, and an organic electroluminescent device comprising the organic electroluminescent compound and/or the organic electroluminescent material.

The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl(ene)” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms including at least one, preferably 1 to 4 heteroatoms selected from the group consisting of B, N, O, S, Si, P, Se, and Ge, in which the number of the ring backbone carbon atoms is preferably 3 to 30, and more preferably 5 to 25. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s), and may comprise a spiro structure. Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into heteroaryl(ene) with electronic properties and heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quina It may be zolinyl, a substituted or unsubstituted quinoxalinil, a substituted or unsubstituted quinolyl, etc. Heteroaryl(ene), which has hole characteristics, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl. Herein, the term “a fused ring of (C3-C30) aliphatic ring and (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatom selected from B, N, O, S, Si, and P, preferably at least one heteroatom selected from N, O, and S. The term “Halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si, and P, preferably, N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as one substituent in which two heteroaryls are connected. The substituted aryl(ene), the substituted heteroaryl, the substituted dibenzofuranyl, the substituted dibenzothiophenyl, or the substituted carbazolyl in the formulas of the present disclosure, each independently is substituted with at least one selected from the group consisting of deuterium; halogen; cyano; carboxyl; nitro; hydroxyl; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one (C6-C30)aryl; (C6-C30)aryl unsubstituted or substituted with at least one of (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; tri(C6-C30)arylgermanyl; amino; mono- or di- (C1-C30)alkylamino; mono- or di- (C2-C30)alkenylamino; mono- or di- (C6-C30)arylamino unsubstituted or substituted with (C1-C30)alkyl; mono- or di- (3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl, etc.

In formulas of the present disclosure, when a plurality of substituents represented by the same symbol are present, each of the substituents represented by the same symbol may be the same or different.

Hereinafter, the plurality of host materials according to one embodiment will be described.

The plurality of host materials according to one embodiment comprise at least one first host compound and at least one second host compound, wherein the first host compound is represented by Formula 1, the second host compound is represented by Formula 2, and at least one of the first host compound and the second host compound comprises at least one deuterium.

The first host compound as the host material according to one embodiment may be represented by the following Formula 1.

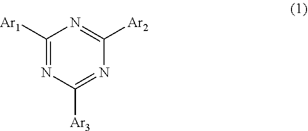

In Formula 1,
Ar₁ to Ar₃ each independently represent, a substituted or unsubstituted (C6-C30)aryl or a substituted or unsubstituted (3- to 30-membered)heteroaryl; provided that at least one of Ar₁ to Ar₃ is a carbazole group represented by the following Formula 1-1;

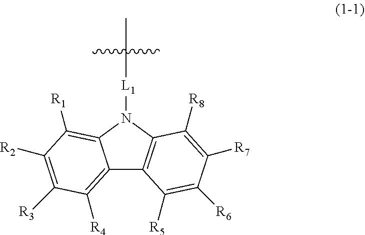

In Formula 1-1,
L₁ represents a single bond or (C6-C30)arylene unsubstituted or substituted with deuterium;
R₁ to R₈ each independently represent, hydrogen, deuterium, or (C6-C30)aryl unsubstituted or substituted with deuterium, (C1-C30)alkyl, (C6-C30)aryl, or a combination thereof; provided that at least one of R₁ to R₄ is represented by the following formulas 1-11 or 1-12;

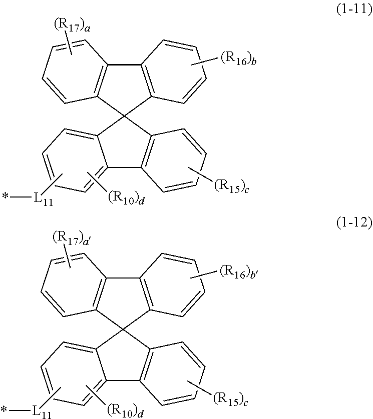

In formulas 1-11 and 1-12,
L₁₁ represents a single bond or (C6-C30)arylene unsubstituted or substituted with deuterium;
R₁₀ and R₁₅ to R₁₇ each independently represent, hydrogen, deuterium, or (C6-C30)aryl unsubstituted or substituted with deuterium;
a to c each independently represent, an integer of 1 to 4; d is an integer of 1 to 3; and a′ and b′ each independently represent, an integer of 1 to 5; when a to d, a′, and b′ are 2 or more, each of R₁₀ and each of R₁₅ to each of R₇ may be the same or different; and
* represents a linking site with R₁ to R₄.

In one embodiment, Ar₁ to Ar₃ each independently may be, a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₁ to Ar₃ each independently may be, phenyl unsubstituted or substituted with deuterium, biphenyl unsubstituted or substituted with deuterium, terphenyl unsubstituted or substituted with deuterium, quarterphenyl unsubstituted or substituted with deuterium, naphthyl unsubstituted or substituted with deuterium, phenylnaphthyl unsubstituted or substituted with deuterium, naphthylphenyl unsubstituted or substituted with deuterium, dibenzofuranyl unsubstituted or substituted with deuterium, dibenzothiophenyl unsubstituted or substituted with deuterium, carbazolyl unsubstituted or substituted with deuterium, or a combination thereof. For example, Ar₁ to Ar₃ each independently may be, a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl a substituted or unsubstituted carbazolyl, or a substituted or unsubstituted dibenzofuranyl.

In one embodiment, at least one of Ar₁ to Ar₃ is a carbazole group represented by Formula 1-1.

In one embodiment, L₁ may be a single bond or (C6-C25)arylene unsubstituted or substituted with deuterium, preferably a single bond or (C6-C18)arylene unsubstituted or substituted with deuterium, more preferably a single bond or an unsubstituted (C6-C18)arylene. For example, L₁ may be a single bond, phenylene unsubstituted or substituted with deuterium, or naphthylene unsubstituted or substituted with deuterium.

In one embodiment, R₁ to R₈ each independently may be hydrogen, deuterium, or (C6-C30)aryl unsubstituted or substituted with deuterium, (C1-C10)alkyl, (C6-C30)aryl, or a combination thereof, preferably hydrogen, deuterium, or (C6-C25)aryl unsubstituted or substituted with deuterium, (C1-C10)alkyl, (C6-C25)aryl, or a combination thereof, more preferably hydrogen, deuterium, or (C6-C25)aryl unsubstituted or substituted with at least one of deuterium, (C1-C4)alkyl, and (C6-C18)aryl. For example, R₁ to R₈ each independently may be hydrogen, deuterium, phenyl unsubstituted or substituted with deuterium, biphenyl unsubstituted or substituted with deuterium, terphenyl unsubstituted or substituted with deuterium, quarterphenyl unsubstituted or substituted with deuterium, naphthyl unsubstituted or substituted with deuterium, phenylnaphthyl unsubstituted or substituted with deuterium, naphthylphenyl unsubstituted or substituted with deuterium, or a combination thereof. For example, R₁ to R₈ each independently may be hydrogen, a substituted or unsubstituted phenyl, a substituted or unsubstituted diphenylfluorenyl, or a substituted or unsubstituted spirobifluorenyl.

In one embodiment, at least one of R₁ to R₄ may be spirobifluorenyl represented by Formula 1-11 or diphenylfluorenyl represented by Formula 1-12.

In one embodiment, L₁₁ may be a single bond.

In one embodiment, R₁₀ and R₁₅ to R₁₇ each independently may be hydrogen, deuterium, or (C6-C25)aryl unsubstituted or substituted with deuterium, preferably hydrogen, deuterium, or (C6-C18)aryl unsubstituted or substituted with deuterium, more preferably hydrogen or an unsubstituted (C6-C18)aryl. For example, R₁₀ and R₁₅ to R₁₇ each independently may be hydrogen or phenyl unsubstituted or substituted with deuterium. In one embodiment, R₂ in Formula 1-1 may be represented by the following formulas 1-11-1 or 1-12-1.

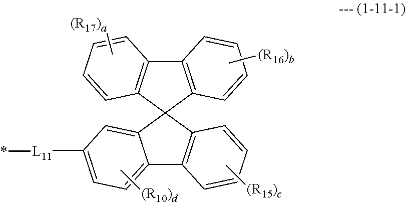

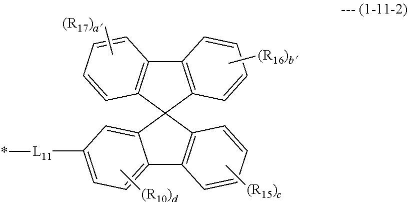

In formulas 1-11-1 and 1-12-1,
L₁₁, R₁₀, R₁₅ to R₁₇, a to d, a′, and b′ are as defined in Formula 1-1.

According to one embodiment, the first host compound represented by Formula 1 may be more specifically illustrated by the following compounds, but is not limited thereto.

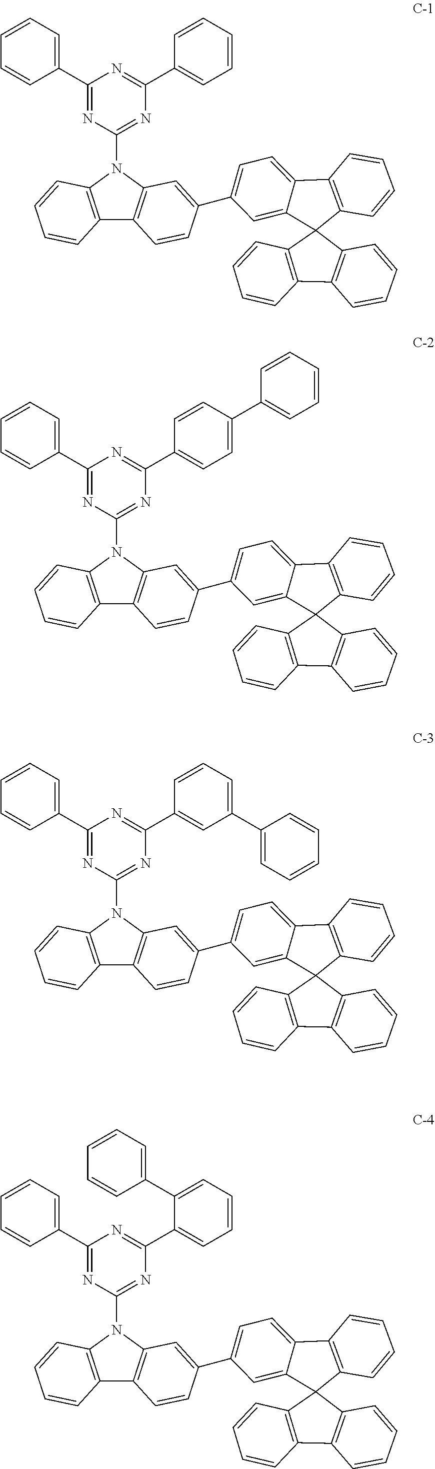 [SMILES: c1ccc(-c2nc(-c3ccccc3)nc(-n3c4ccccc4c4ccc(-c5ccc6c(c5)C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2nc(-c3ccccc3-c3ccccc3)nc(-n3c4ccccc4c4ccc(-c5ccc6c(c5)C5(c7ccccc7-c7ccccc75)c5ccccc5-6)cc43)n2)cc1]

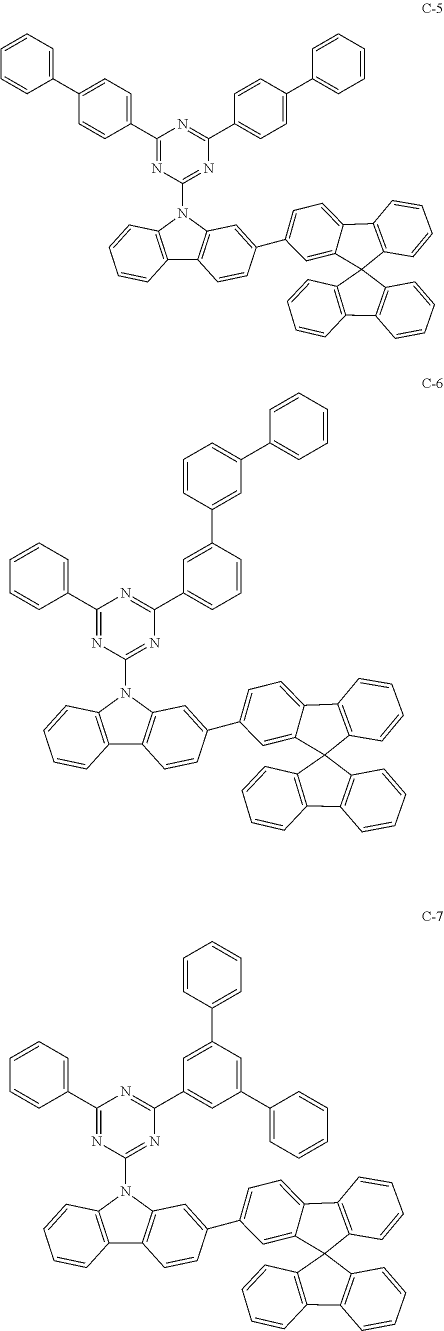 [SMILES: c1ccc(-c2ccc(-c3nc(-c4ccc(-c5ccccc5)cc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)c2)cc1 ; c1ccc(-c2cc(-c3ccccc3)cc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1]

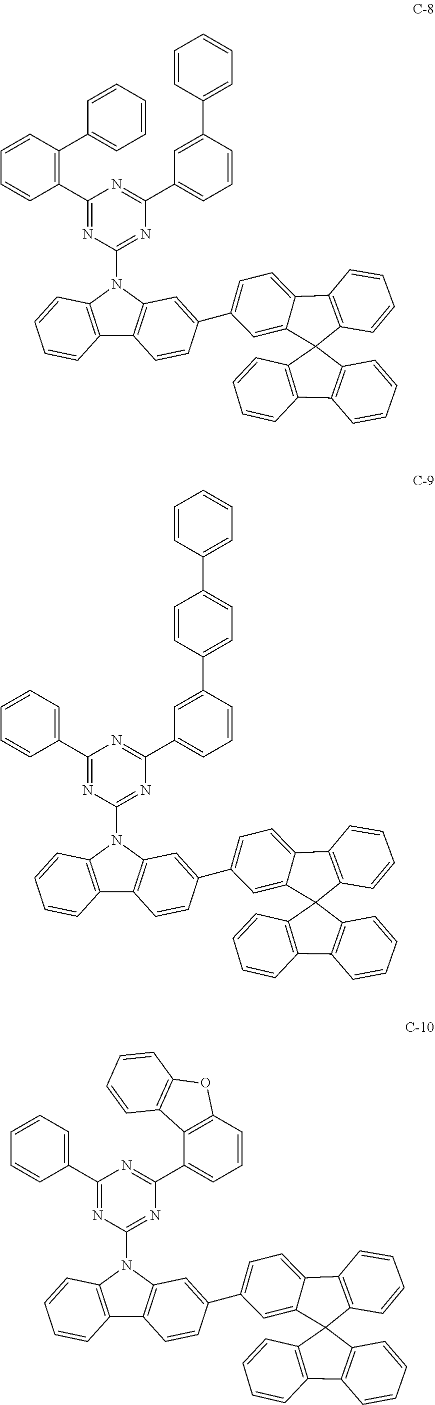 [SMILES: c1ccc(-c2cccc(-c3nc(-c4ccccc4-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2ccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)cc2)cc1 ; c1ccc(-c2nc(-c3cccc4oc5ccccc5c34)nc(-n3c4ccccc4c4ccc(-c5ccc6c(c5)C5(c7ccccc7-c7ccccc75)c5ccccc5-6)cc43)n2)cc1]

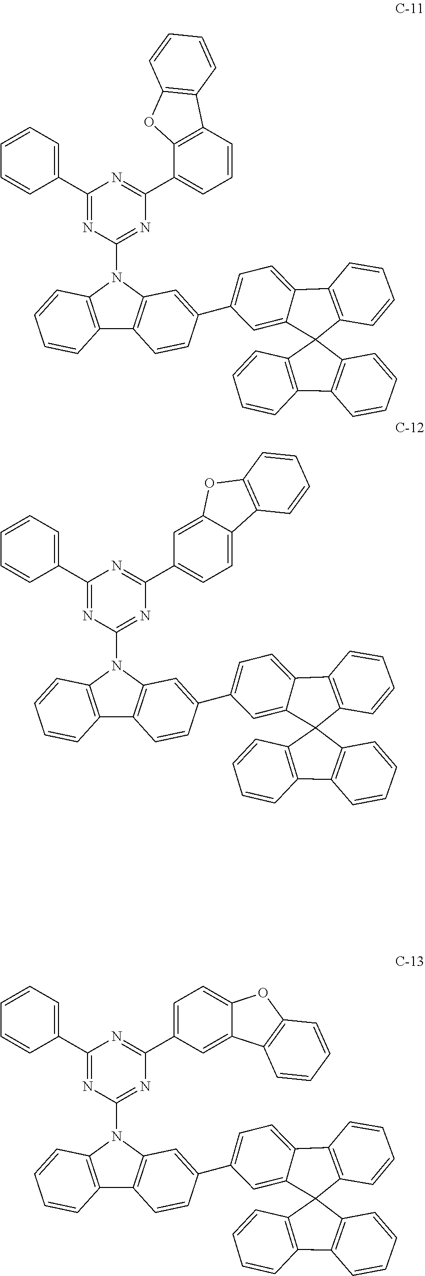

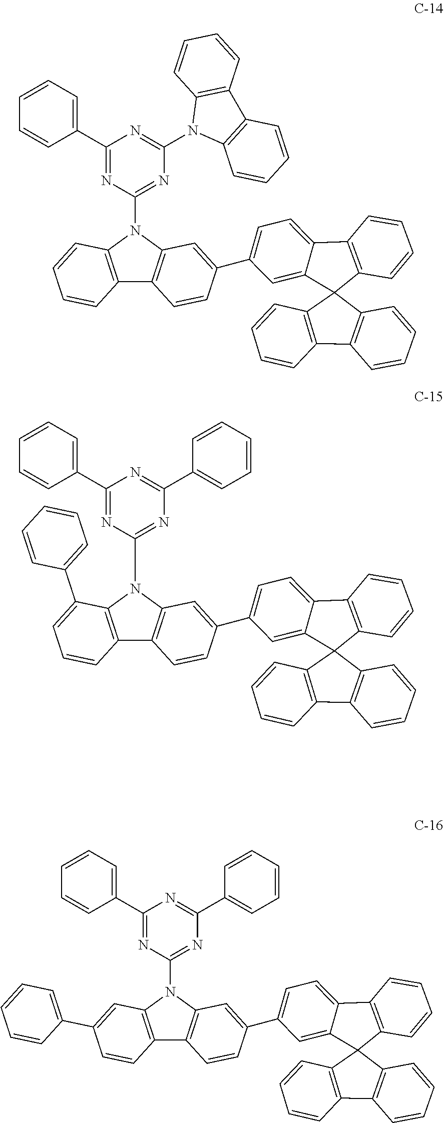 [SMILES: c1ccc(-c2nc(-n3c4ccccc4c4ccccc43)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccccc3)nc(-n3c4cc(-c5ccc6c(c5)C5(c7ccccc7-c7ccccc75)c5ccccc5-6)ccc4c4cccc(-c5ccccc5)c43)n2)cc1 ; c1ccc(-c2ccc3c4ccc(-c5ccc6c(c5)C5(c7ccccc7-c7ccccc75)c5ccccc5-6)cc4n(-c4nc(-c5ccccc5)nc(-c5ccccc5)n4)c3c2)cc1]

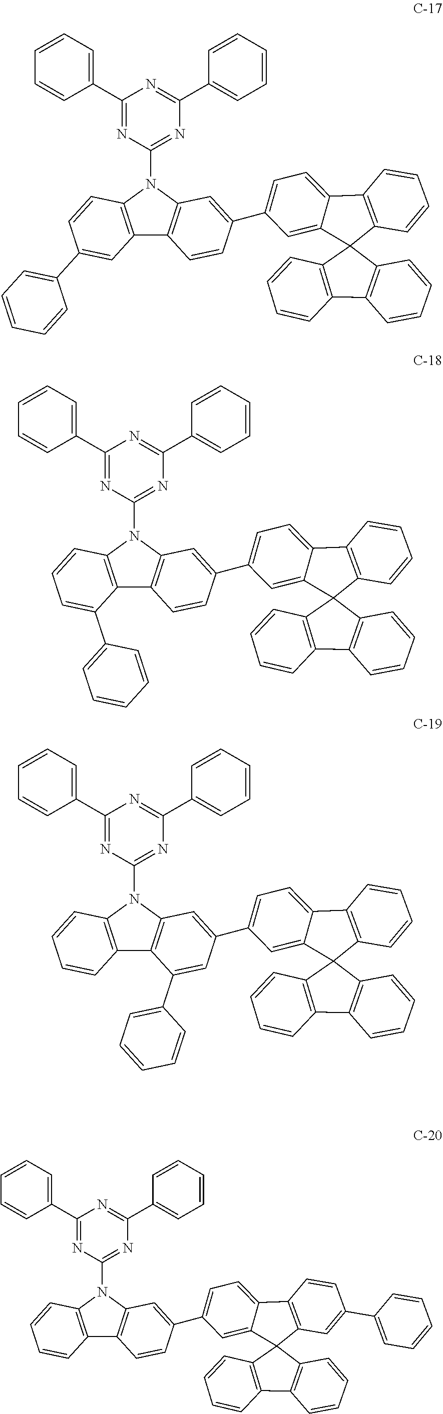 [SMILES: c1ccc(-c2ccc3c(c2)c2ccc(-c4ccc5c(c4)C4(c6ccccc6-c6ccccc64)c4ccccc4-5)cc2n3-c2nc(-c3ccccc3)nc(-c3ccccc3)n2)cc1 ; c1ccc(-c2nc(-c3ccccc3)nc(-n3c4cc(-c5ccc6c(c5)C5(c7ccccc7-c7ccccc75)c5ccccc5-6)ccc4c4c(-c5ccccc5)cccc43)n2)cc1 ; c1ccc(-c2nc(-c3ccccc3)nc(-n3c4ccccc4c4c(-c5ccccc5)cc(-c5ccc6c(c5)C5(c7ccccc7-c7ccccc75)c5ccccc5-6)cc43)n2)cc1 ; c1ccc(-c2ccc3c(c2)C2(c4ccccc4-c4ccccc42)c2cc(-c4ccc5c6ccccc6n(-c6nc(-c7ccccc7)nc(-c7ccccc7)n6)c5c4)ccc2-3)cc1]

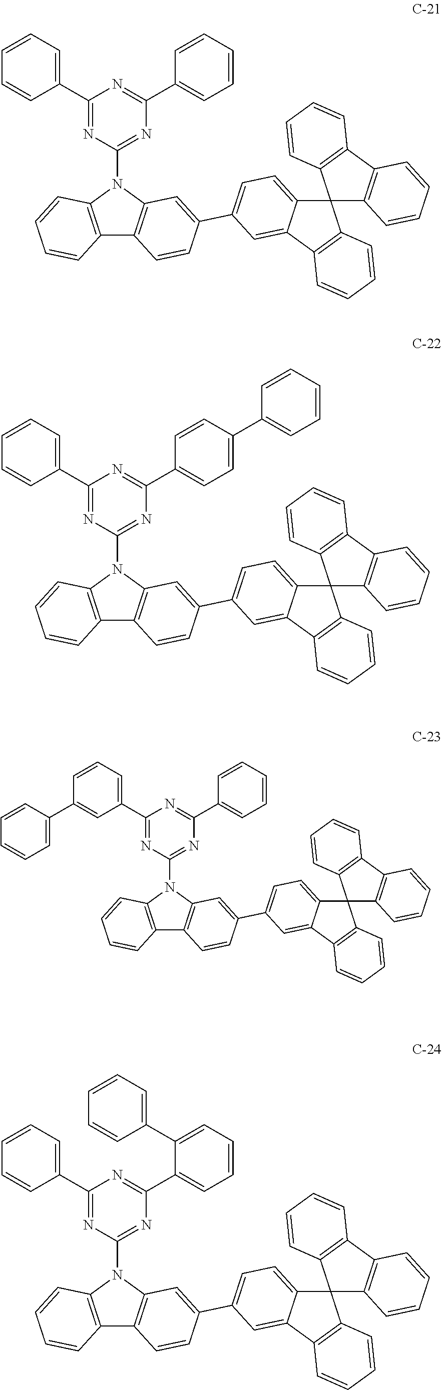 [SMILES: c1ccc(-c2nc(-c3ccccc3)nc(-n3c4ccccc4c4ccc(-c5ccc6c(c5)-c5ccccc5C65c6ccccc6-c6ccccc65)cc43)n2)cc1 ; c1ccc(-c2ccc(-c3nc(-c4ccccc4)nc(-n4c5ccccc5c5ccc(-c6ccc7c(c6)-c6ccccc6C76c7ccccc7-c7ccccc76)cc54)n3)cc2)cc1 ; c1ccc(-c2cccc(-c3nc(-c4ccccc4)nc(-n4c5ccccc5c5ccc(-c6ccc7c(c6)-c6ccccc6C76c7ccccc7-c7ccccc76)cc54)n3)c2)cc1 ; c1ccc(-c2nc(-c3ccccc3-c3ccccc3)nc(-n3c4ccccc4c4ccc(-c5ccc6c(c5)-c5ccccc5C65c6ccccc6-c6ccccc65)cc43)n2)cc1]

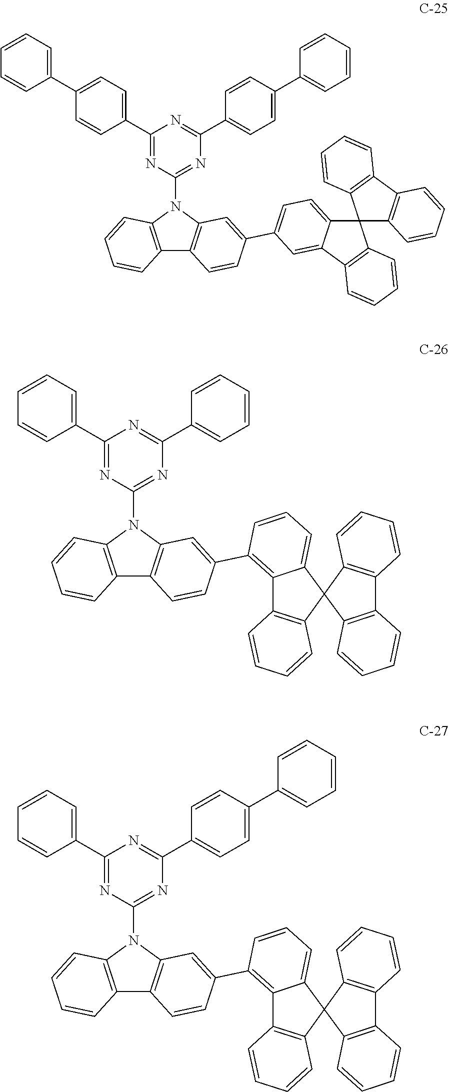 [SMILES: c1ccc(-c2ccc(-c3nc(-c4ccc(-c5ccccc5)cc4)nc(-n4c5ccccc5c5ccc(-c6ccc7c(c6)-c6ccccc6C76c7ccccc7-c7ccccc76)cc54)n3)cc2)cc1 ; c1ccc(-c2nc(-c3ccccc3)nc(-n3c4ccccc4c4ccc(-c5cccc6c5-c5ccccc5C65c6ccccc6-c6ccccc65)cc43)n2)cc1 ; c1ccc(-c2ccc(-c3nc(-c4ccccc4)nc(-n4c5ccccc5c5ccc(-c6cccc7c6-c6ccccc6C76c7ccccc7-c7ccccc76)cc54)n3)cc2)cc1]

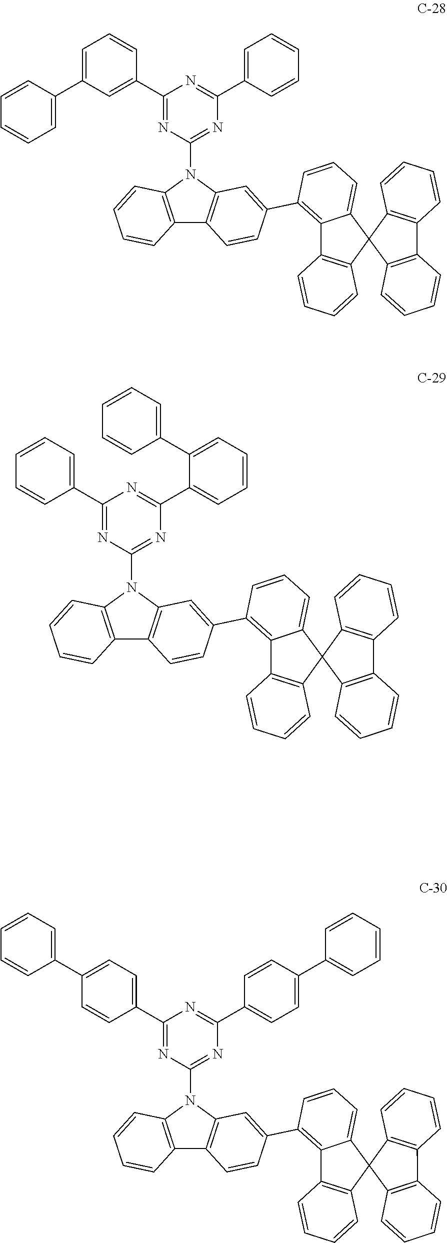 [SMILES: c1ccc(-c2cccc(-c3nc(-c4ccccc4)nc(-n4c5ccccc5c5ccc(-c6cccc7c6-c6ccccc6C76c7ccccc7-c7ccccc76)cc54)n3)c2)cc1 ; c1ccc(-c2nc(-c3ccccc3-c3ccccc3)nc(-n3c4ccccc4c4ccc(-c5cccc6c5-c5ccccc5C65c6ccccc6-c6ccccc65)cc43)n2)cc1 ; c1ccc(-c2ccc(-c3nc(-c4ccc(-c5ccccc5)cc4)nc(-n4c5ccccc5c5ccc(-c6cccc7c6-c6ccccc6C76c7ccccc7-c7ccccc76)cc54)n3)cc2)cc1]

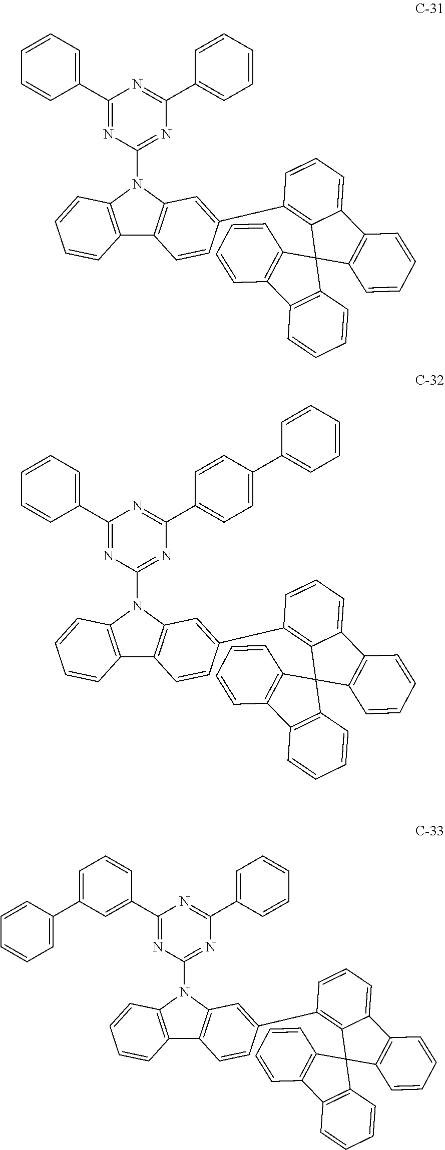 [SMILES: c1ccc(-c2nc(-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6cccc7c6C6(c8ccccc8-c8ccccc86)c6ccccc6-7)cc54)n3)cc2)cc1 ; c1ccc(-c2cccc(-c3nc(-c4ccccc4)nc(-n4c5ccccc5c5ccc(-c6cccc7c6C6(c8ccccc8-c8ccccc86)c6ccccc6-7)cc54)n3)c2)cc1]

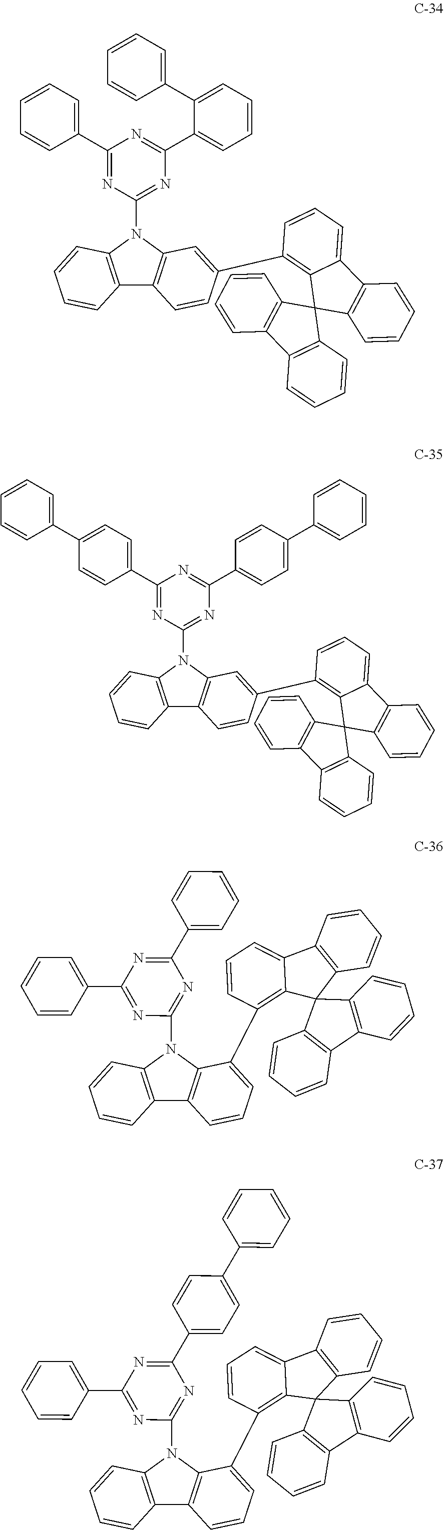 [SMILES: c1ccc(-c2nc(-c3ccccc3-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccc(-c5ccccc5)cc4)nc(-n4c5ccccc5c5ccc(-c6cccc7c6C6(c8ccccc8-c8ccccc86)c6ccccc6-7)cc54)n3)cc2)cc1 ; c1ccc(-c2nc(-c3ccccc3)nc(-n3c4ccccc4c4cccc(-c5cccc6c5C5(c7ccccc7-c7ccccc75)c5ccccc5-6)c43)n2)cc1 ; c1ccc(-c2ccc(-c3nc(-c4ccccc4)nc(-n4c5ccccc5c5cccc(-c6cccc7c6C6(c8ccccc8-c8ccccc86)c6ccccc6-7)c54)n3)cc2)cc1]

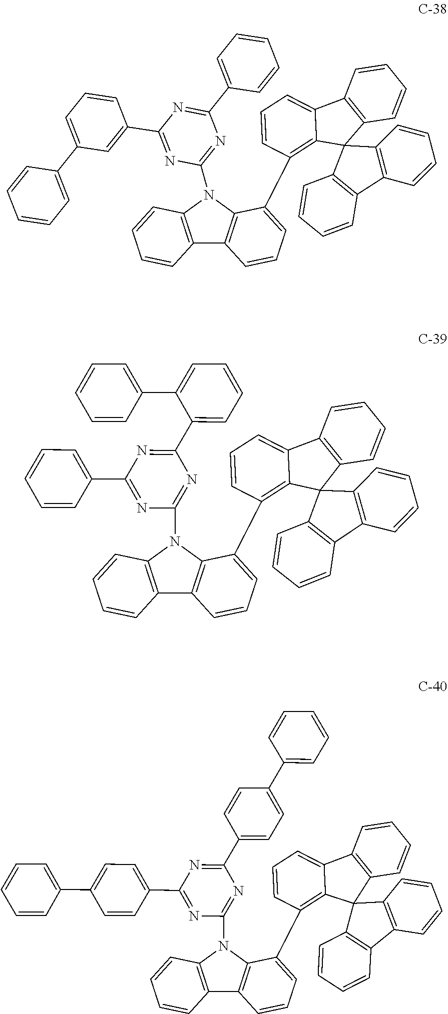 [SMILES: c1ccc(-c2cccc(-c3nc(-c4ccccc4)nc(-n4c5ccccc5c5cccc(-c6cccc7c6C6(c8ccccc8-c8ccccc86)c6ccccc6-7)c54)n3)c2)cc1 ; c1ccc(-c2nc(-c3ccccc3-c3ccccc3)nc(-n3c4ccccc4c4cccc(-c5cccc6c5C5(c7ccccc7-c7ccccc75)c5ccccc5-6)c43)n2)cc1 ; c1ccc(-c2ccc(-c3nc(-c4ccc(-c5ccccc5)cc4)nc(-n4c5ccccc5c5cccc(-c6cccc7c6C6(c8ccccc8-c8ccccc86)c6ccccc6-7)c54)n3)cc2)cc1]

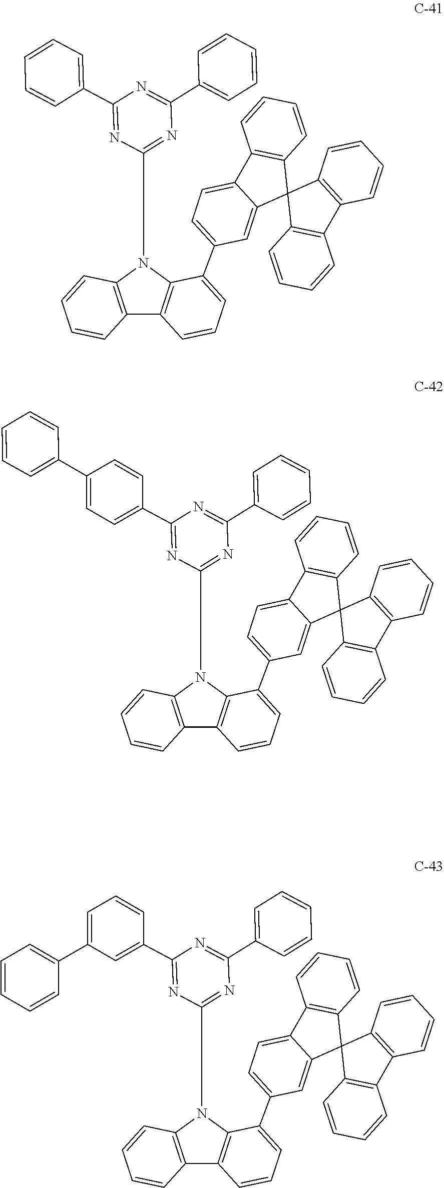 [SMILES: c1ccc(-c2nc(-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccccc4)nc(-n4c5ccccc5c5cccc(-c6ccc7c(c6)C6(c8ccccc8-c8ccccc86)c6ccccc6-7)c54)n3)cc2)cc1 ; c1ccc(-c2cccc(-c3nc(-c4ccccc4)nc(-n4c5ccccc5c5cccc(-c6ccc7c(c6)C6(c8ccccc8-c8ccccc86)c6ccccc6-7)c54)n3)c2)cc1]

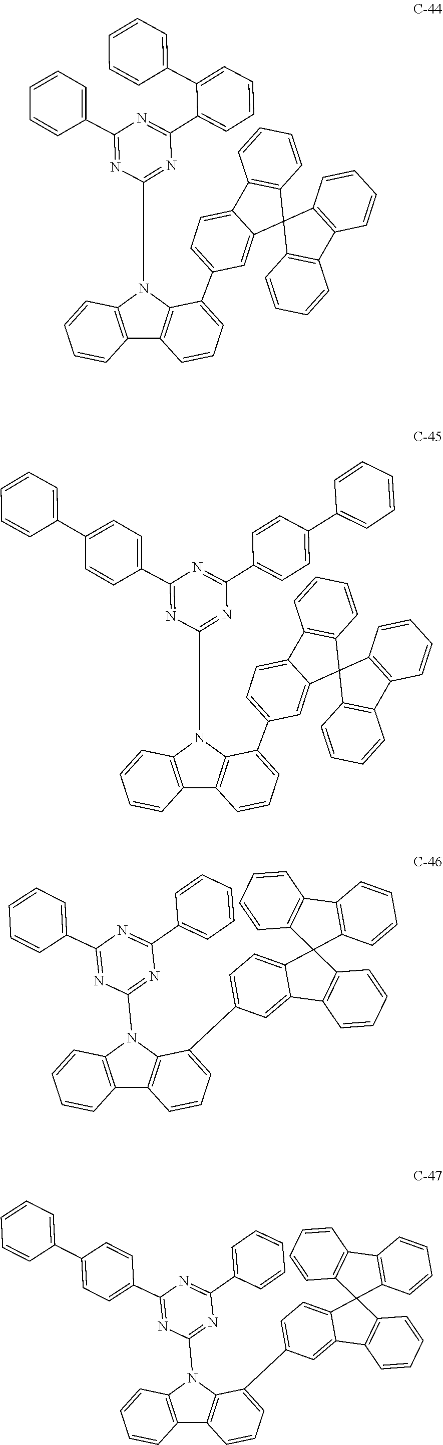 [SMILES: c1ccc(-c2nc(-c3ccccc3-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccc(-c5ccccc5)cc4)nc(-n4c5ccccc5c5cccc(-c6ccc7c(c6)C6(c8ccccc8-c8ccccc86)c6ccccc6-7)c54)n3)cc2)cc1 ; c1ccc(-c2nc(-c3ccccc3)nc(-n3c4ccccc4c4cccc(-c5ccc6c(c5)-c5ccccc5C65c6ccccc6-c6ccccc65)c43)n2)cc1 ; c1ccc(-c2ccc(-c3nc(-c4ccccc4)nc(-n4c5ccccc5c5cccc(-c6ccc7c(c6)-c6ccccc6C76c7ccccc7-c7ccccc76)c54)n3)cc2)cc1]

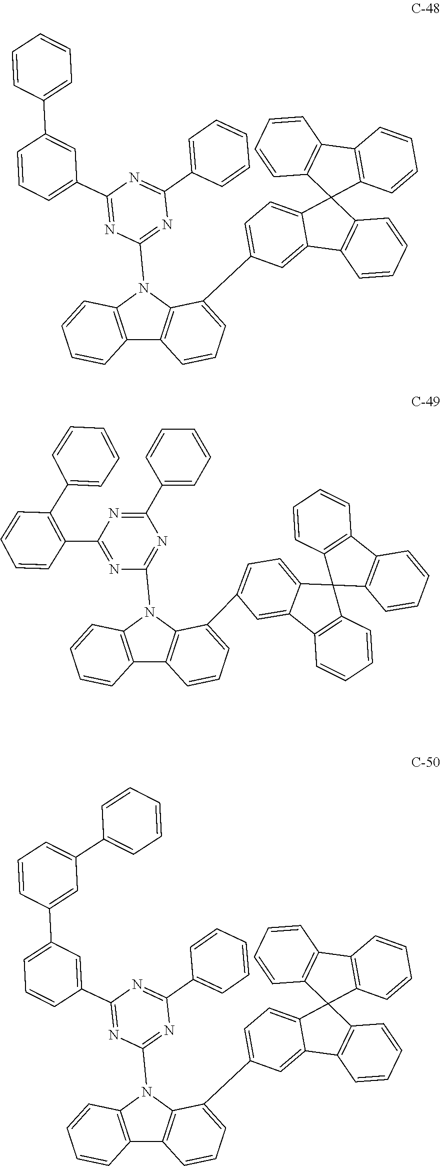 [SMILES: c1ccc(-c2cccc(-c3nc(-c4ccccc4)nc(-n4c5ccccc5c5cccc(-c6ccc7c(c6)-c6ccccc6C76c7ccccc7-c7ccccc76)c54)n3)c2)cc1 ; c1ccc(-c2nc(-c3ccccc3-c3ccccc3)nc(-n3c4ccccc4c4cccc(-c5ccc6c(c5)-c5ccccc5C65c6ccccc6-c6ccccc65)c43)n2)cc1 ; c1ccc(-c2cccc(-c3cccc(-c4nc(-c5ccccc5)nc(-n5c6ccccc6c6cccc(-c7ccc8c(c7)-c7ccccc7C87c8ccccc8-c8ccccc87)c65)n4)c3)c2)cc1]

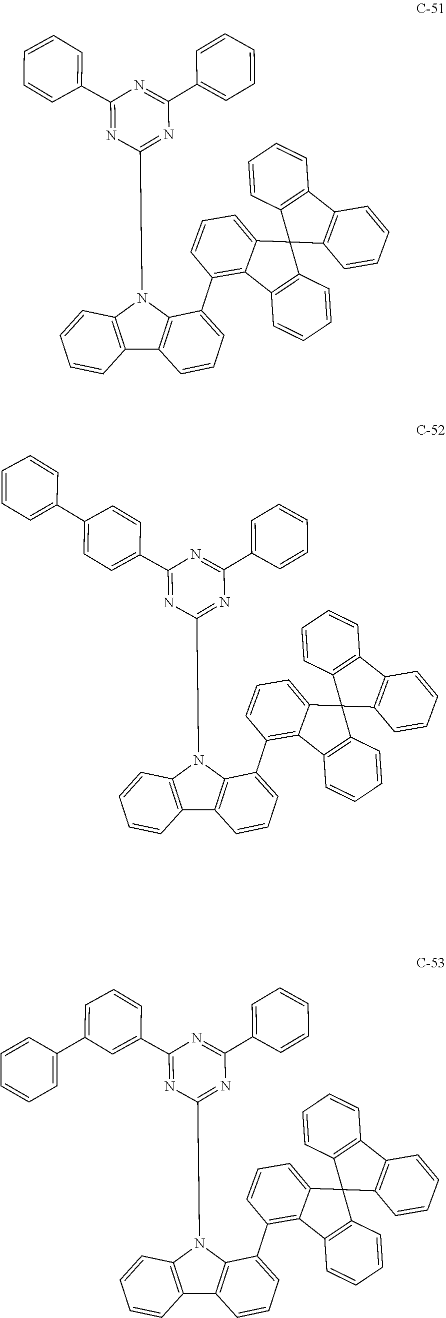 [SMILES: c1ccc(-c2nc(-c3ccccc3)nc(-n3c4ccccc4c4cccc(-c5cccc6c5-c5ccccc5C65c6ccccc6-c6ccccc65)c43)n2)cc1 ; c1ccc(-c2ccc(-c3nc(-c4ccccc4)nc(-n4c5ccccc5c5cccc(-c6cccc7c6-c6ccccc6C76c7ccccc7-c7ccccc76)c54)n3)cc2)cc1 ; c1ccc(-c2cccc(-c3nc(-c4ccccc4)nc(-n4c5ccccc5c5cccc(-c6cccc7c6-c6ccccc6C76c7ccccc7-c7ccccc76)c54)n3)c2)cc1]

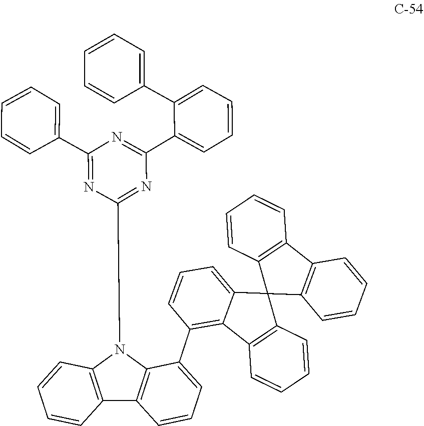 [SMILES: c1ccc(-c2nc(-c3ccccc3-c3ccccc3)nc(-n3c4ccccc4c4cccc(-c5cccc6c5-c5ccccc5C65c6ccccc6-c6ccccc65)c43)n2)cc1]

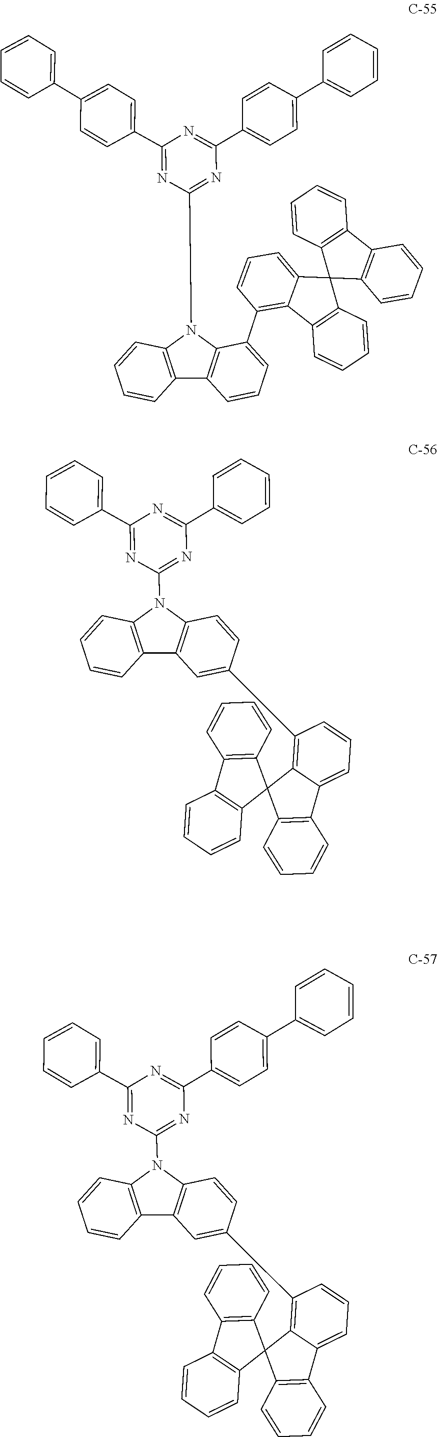 [SMILES: c1ccc(-c2ccc(-c3nc(-c4ccc(-c5ccccc5)cc4)nc(-n4c5ccccc5c5cccc(-c6cccc7c6-c6ccccc6C76c7ccccc7-c7ccccc76)c54)n3)cc2)cc1 ; c1ccc(-c2nc(-c3ccccc3)nc(-n3c4ccccc4c4cc(-c5cccc6c5C5(c7ccccc7-c7ccccc75)c5ccccc5-6)ccc43)n2)cc1 ; c1ccc(-c2ccc(-c3nc(-c4ccccc4)nc(-n4c5ccccc5c5cc(-c6cccc7c6C6(c8ccccc8-c8ccccc86)c6ccccc6-7)ccc54)n3)cc2)cc1]

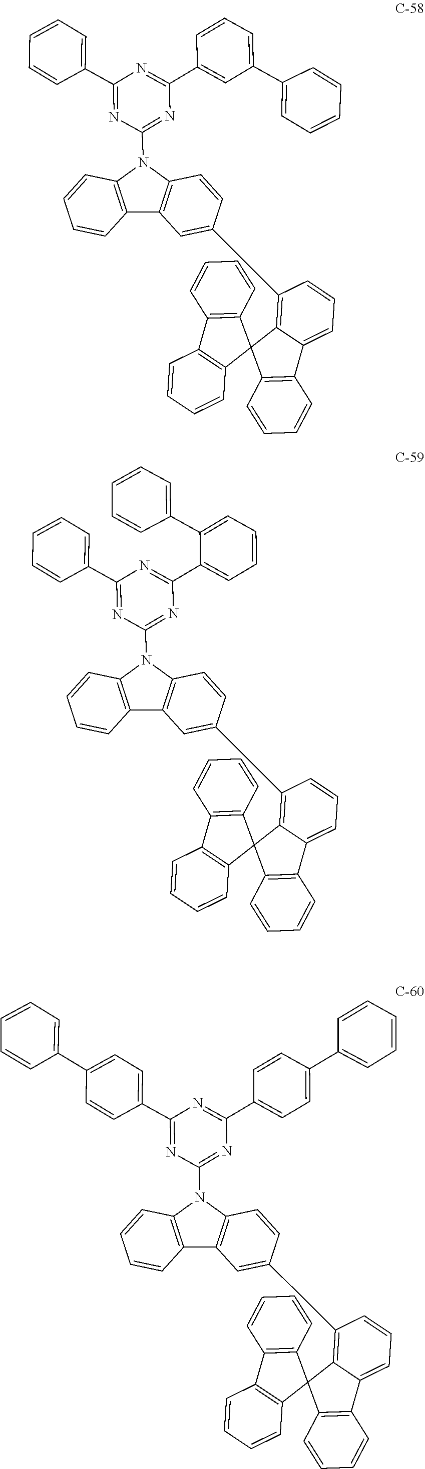 [SMILES: c1ccc(-c2cccc(-c3nc(-c4ccccc4)nc(-n4c5ccccc5c5cc(-c6cccc7c6C6(c8ccccc8-c8ccccc86)c6ccccc6-7)ccc54)n3)c2)cc1 ; c1ccc(-c2nc(-c3ccccc3-c3ccccc3)nc(-n3c4ccccc4c4cc(-c5cccc6c5C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6cccc7c6C6(c8ccccc8-c8ccccc86)c6ccccc6-7)ccc54)n3)cc2)cc1]

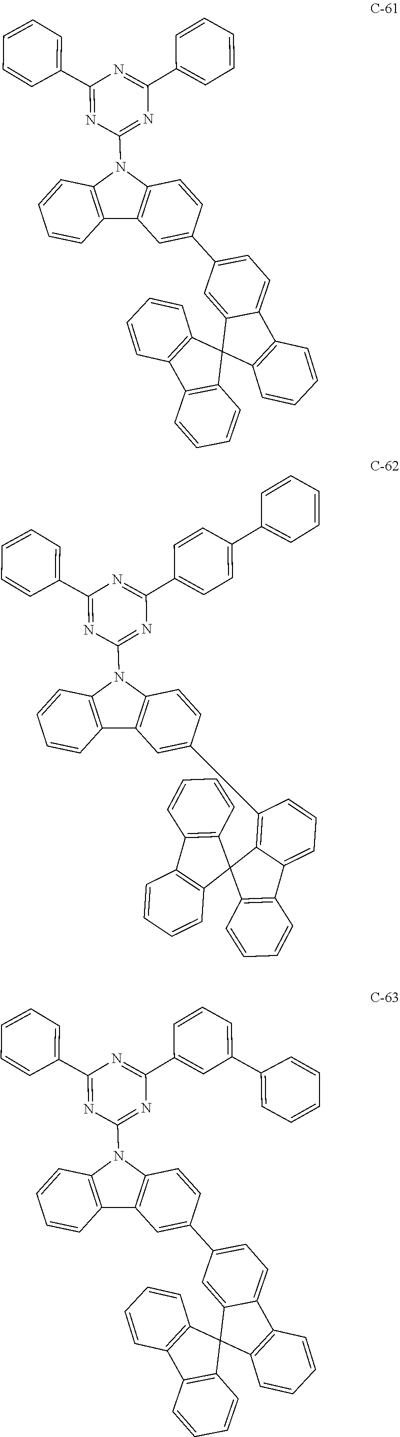 [SMILES: c1ccc(-c2nc(-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccccc4)nc(-n4c5ccccc5c5cc(-c6cccc7c6C6(c8ccccc8-c8ccccc86)c6ccccc6-7)ccc54)n3)cc2)cc1 ; c1ccc(-c2cccc(-c3nc(-c4ccccc4)nc(-n4c5ccccc5c5cc(-c6ccc7c(c6)C6(c8ccccc8-c8ccccc86)c6ccccc6-7)ccc54)n3)c2)cc1]

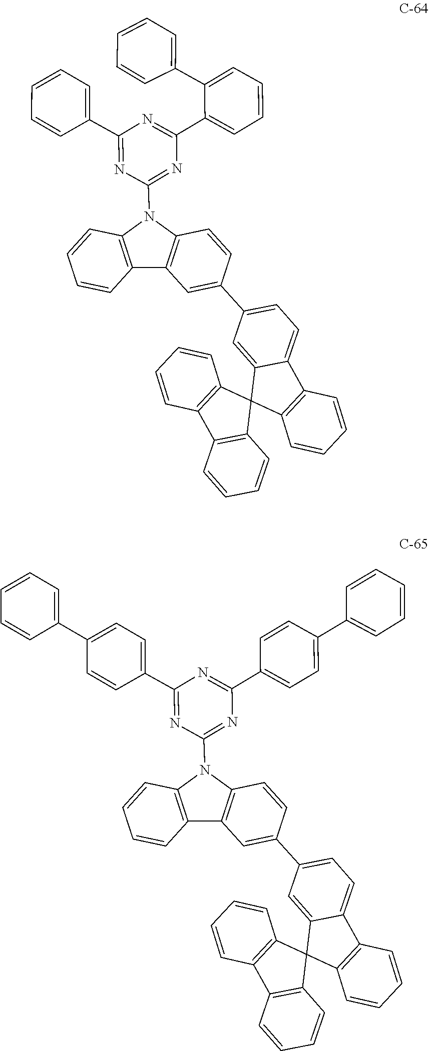 [SMILES: c1ccc(-c2nc(-c3ccccc3-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6ccc7c(c6)C6(c8ccccc8-c8ccccc86)c6ccccc6-7)ccc54)n3)cc2)cc1]

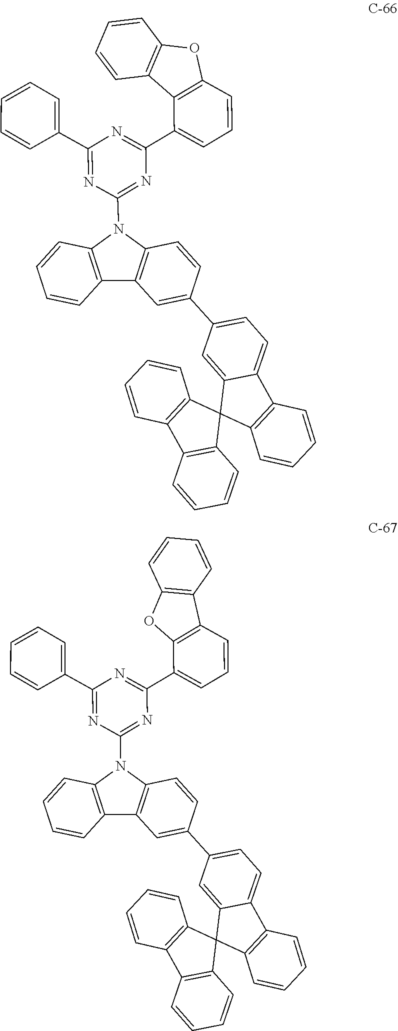

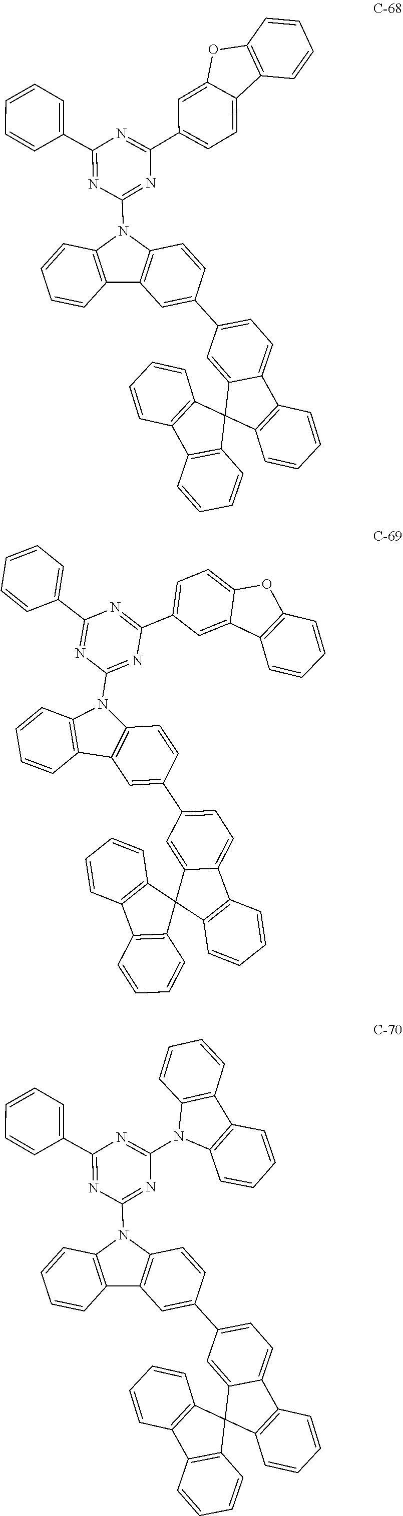 [SMILES: c1ccc(-c2nc(-c3ccc4c(c3)oc3ccccc34)nc(-n3c4ccccc4c4cc(-c5ccc6c(c5)C5(c7ccccc7-c7ccccc75)c5ccccc5-6)ccc43)n2)cc1 ; c1ccc(-c2nc(-c3ccc4oc5ccccc5c4c3)nc(-n3c4ccccc4c4cc(-c5ccc6c(c5)C5(c7ccccc7-c7ccccc75)c5ccccc5-6)ccc43)n2)cc1 ; c1ccc(-c2nc(-n3c4ccccc4c4ccccc43)nc(-n3c4ccccc4c4cc(-c5ccc6c(c5)C5(c7ccccc7-c7ccccc75)c5ccccc5-6)ccc43)n2)cc1]

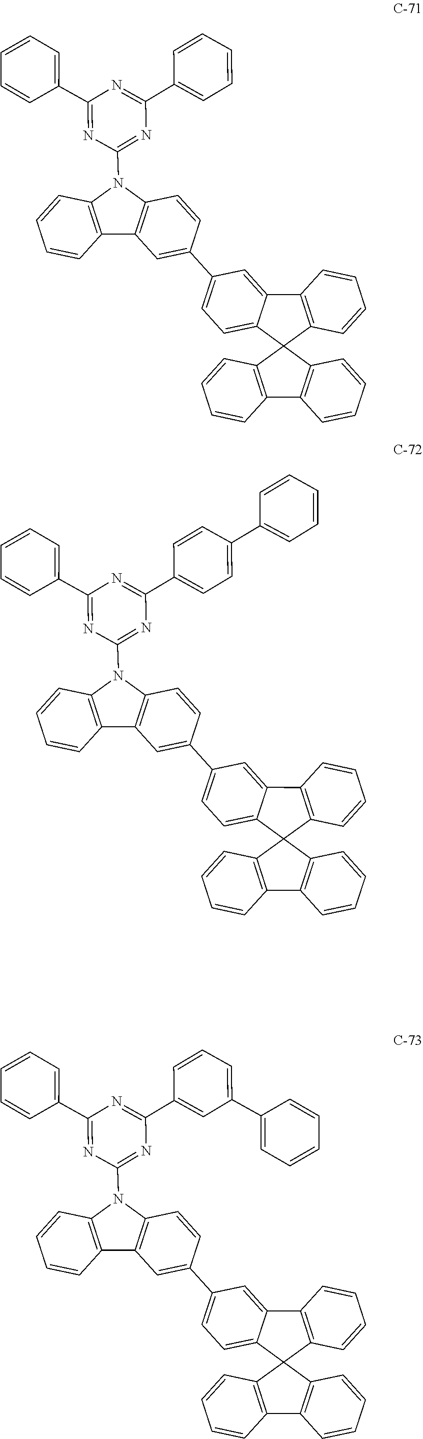 [SMILES: c1ccc(-c2nc(-c3ccccc3)nc(-n3c4ccccc4c4cc(-c5ccc6c(c5)-c5ccccc5C65c6ccccc6-c6ccccc65)ccc43)n2)cc1 ; c1ccc(-c2ccc(-c3nc(-c4ccccc4)nc(-n4c5ccccc5c5cc(-c6ccc7c(c6)-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6ccc7c(c6)-c6ccccc6C76c7ccccc7-c7ccccc76)ccc54)n3)c2)cc1]

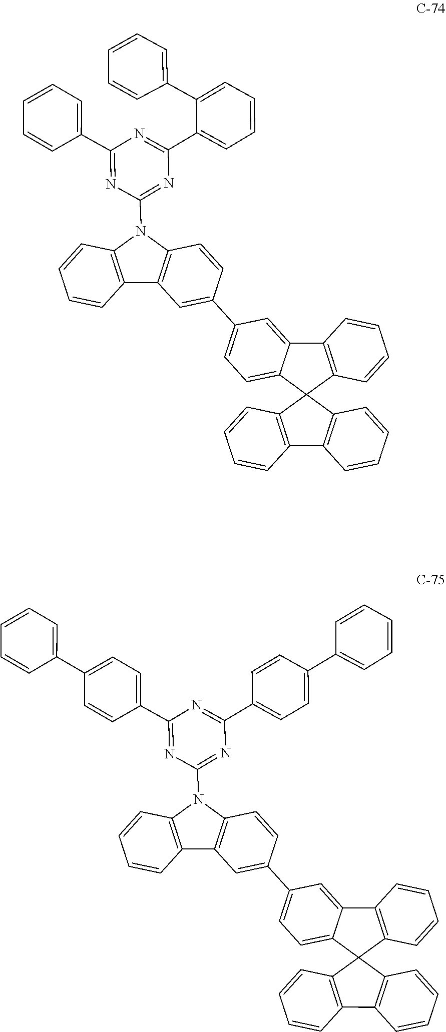 [SMILES: c1ccc(-c2nc(-c3ccccc3-c3ccccc3)nc(-n3c4ccccc4c4cc(-c5ccc6c(c5)-c5ccccc5C65c6ccccc6-c6ccccc65)ccc43)n2)cc1 ; c1ccc(-c2ccc(-c3nc(-c4ccc(-c5ccccc5)cc4)nc(-n4c5ccccc5c5cc(-c6ccc7c(c6)-c6ccccc6C76c7ccccc7-c7ccccc76)ccc54)n3)cc2)cc1]

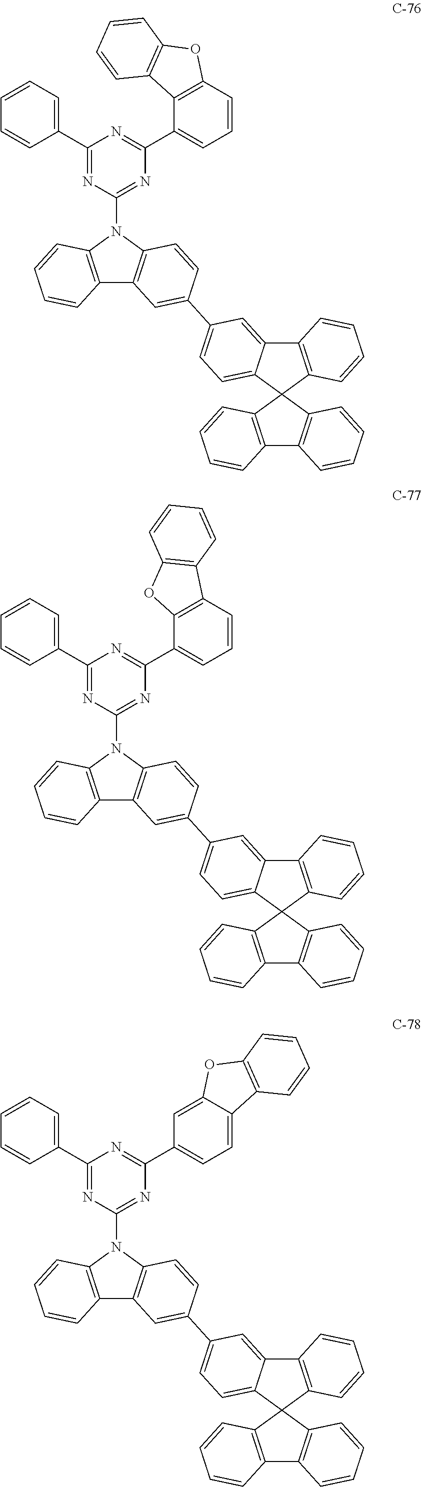

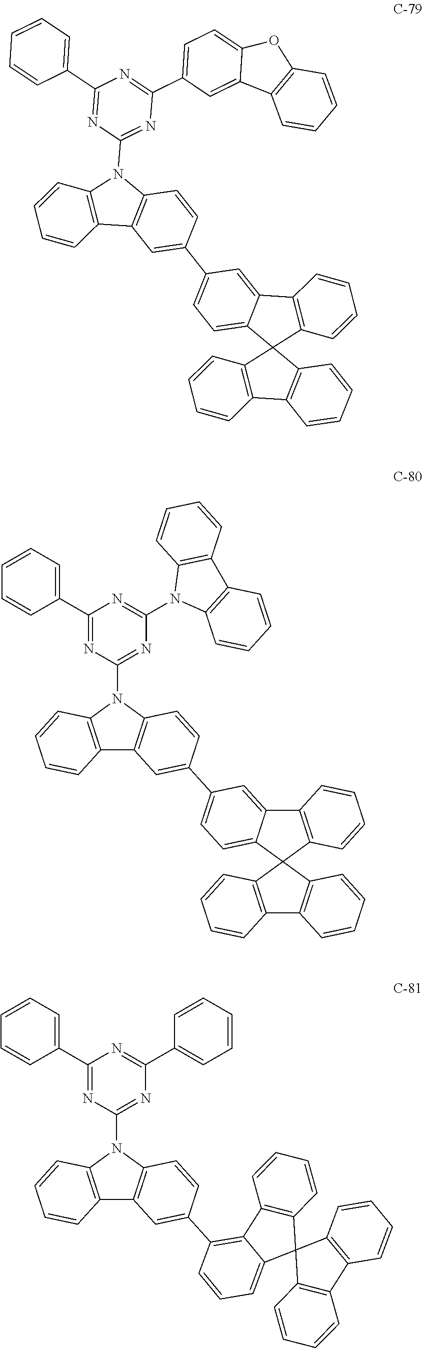 [SMILES: c1ccc(-c2nc(-c3ccc4oc5ccccc5c4c3)nc(-n3c4ccccc4c4cc(-c5ccc6c(c5)-c5ccccc5C65c6ccccc6-c6ccccc65)ccc43)n2)cc1 ; c1ccc(-c2nc(-n3c4ccccc4c4ccccc43)nc(-n3c4ccccc4c4cc(-c5ccc6c(c5)-c5ccccc5C65c6ccccc6-c6ccccc65)ccc43)n2)cc1 ; c1ccc(-c2nc(-c3ccccc3)nc(-n3c4ccccc4c4cc(-c5cccc6c5-c5ccccc5C65c6ccccc6-c6ccccc65)ccc43)n2)cc1]

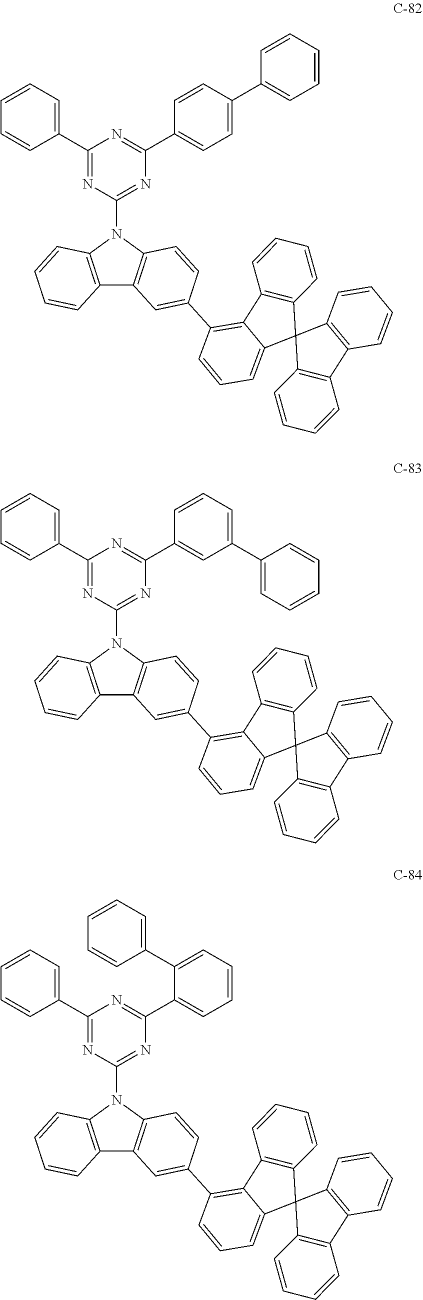 [SMILES: c1ccc(-c2ccc(-c3nc(-c4ccccc4)nc(-n4c5ccccc5c5cc(-c6cccc7c6-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6cccc7c6-c6ccccc6C76c7ccccc7-c7ccccc76)ccc54)n3)c2)cc1 ; c1ccc(-c2nc(-c3ccccc3-c3ccccc3)nc(-n3c4ccccc4c4cc(-c5cccc6c5-c5ccccc5C65c6ccccc6-c6ccccc65)ccc43)n2)cc1]

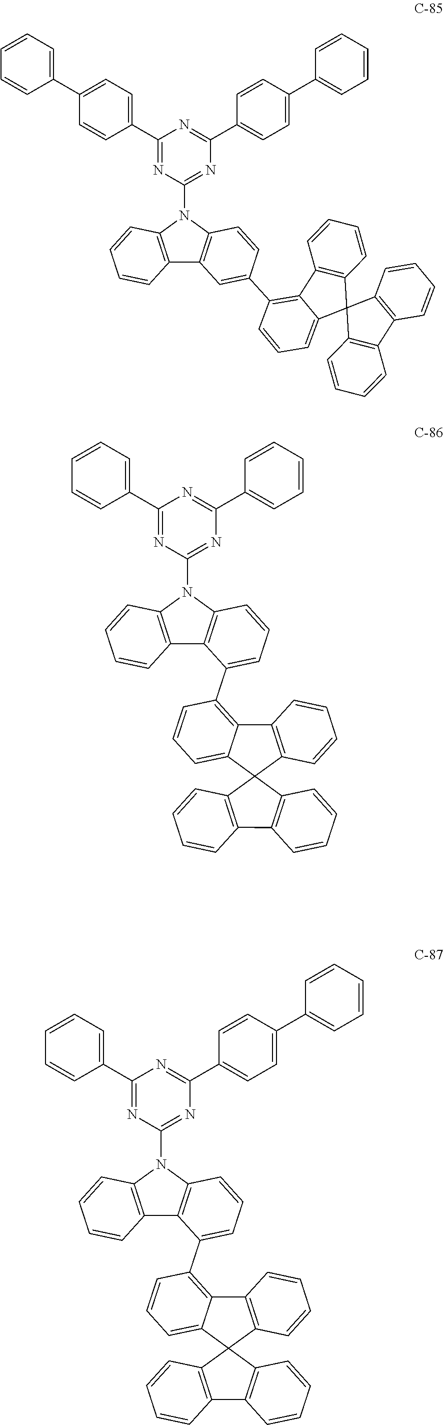 [SMILES: c1ccc(-c2ccc(-c3nc(-c4ccc(-c5ccccc5)cc4)nc(-n4c5ccccc5c5cc(-c6cccc7c6-c6ccccc6C76c7ccccc7-c7ccccc76)ccc54)n3)cc2)cc1 ; c1ccc(-c2nc(-c3ccccc3)nc(-n3c4ccccc4c4c(-c5cccc6c5-c5ccccc5C65c6ccccc6-c6ccccc65)cccc43)n2)cc1 ; c1ccc(-c2ccc(-c3nc(-c4ccccc4)nc(-n4c5ccccc5c5c(-c6cccc7c6-c6ccccc6C76c7ccccc7-c7ccccc76)cccc54)n3)cc2)cc1]

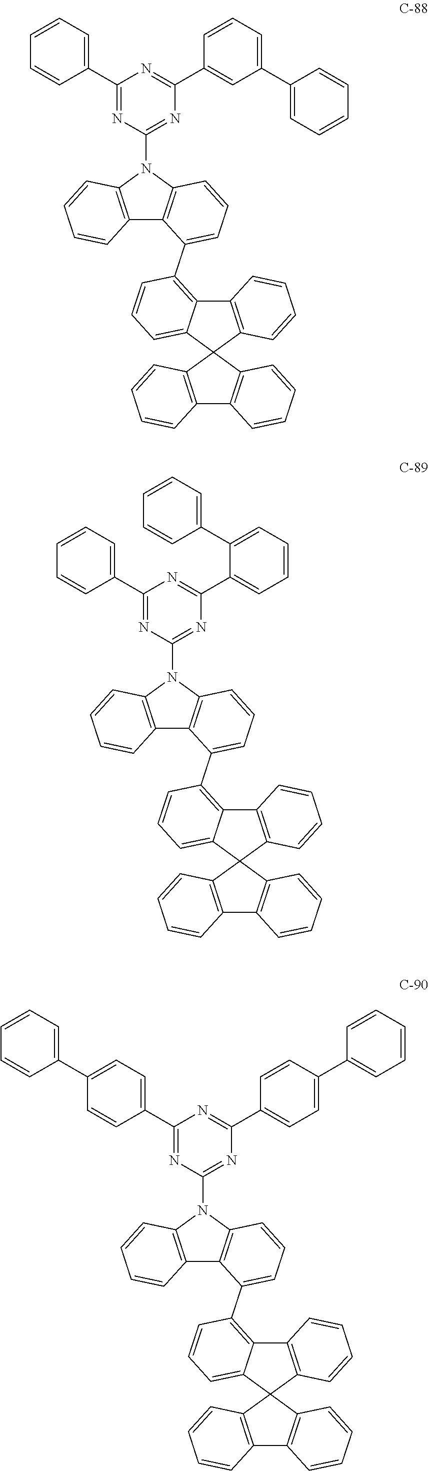 [SMILES: c1ccc(-c2cccc(-c3nc(-c4ccccc4)nc(-n4c5ccccc5c5c(-c6cccc7c6-c6ccccc6C76c7ccccc7-c7ccccc76)cccc54)n3)c2)cc1 ; c1ccc(-c2nc(-c3ccccc3-c3ccccc3)nc(-n3c4ccccc4c4c(-c5cccc6c5-c5ccccc5C65c6ccccc6-c6ccccc65)cccc43)n2)cc1 ; c1ccc(-c2ccc(-c3nc(-c4ccc(-c5ccccc5)cc4)nc(-n4c5ccccc5c5c(-c6cccc7c6-c6ccccc6C76c7ccccc7-c7ccccc76)cccc54)n3)cc2)cc1]

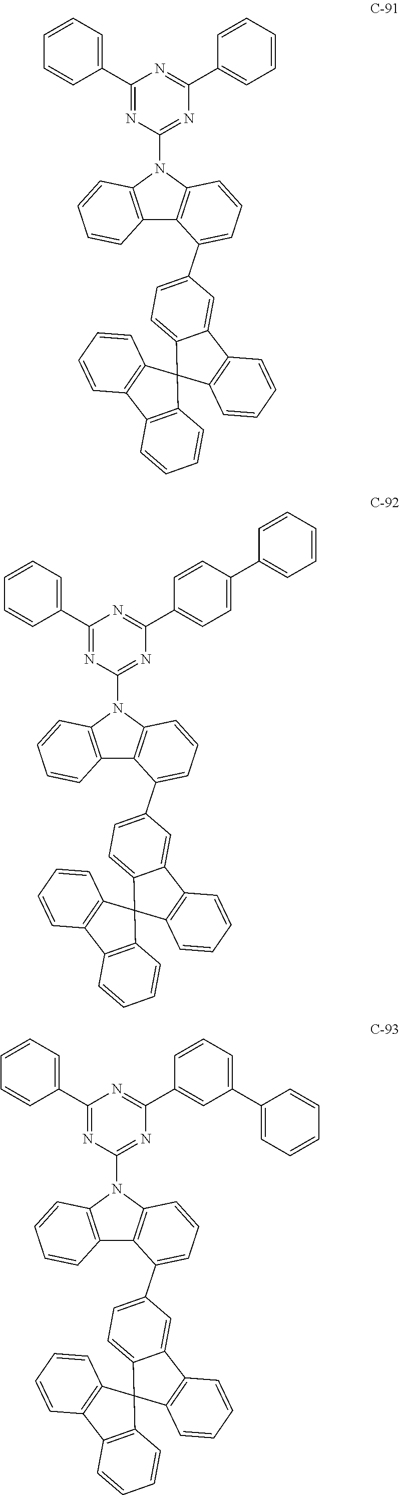 [SMILES: c1ccc(-c2nc(-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccccc4)nc(-n4c5ccccc5c5c(-c6ccc7c(c6)-c6ccccc6C76c7ccccc7-c7ccccc76)cccc54)n3)cc2)cc1 ; c1ccc(-c2cccc(-c3nc(-c4ccccc4)nc(-n4c5ccccc5c5c(-c6ccc7c(c6)-c6ccccc6C76c7ccccc7-c7ccccc76)cccc54)n3)c2)cc1]

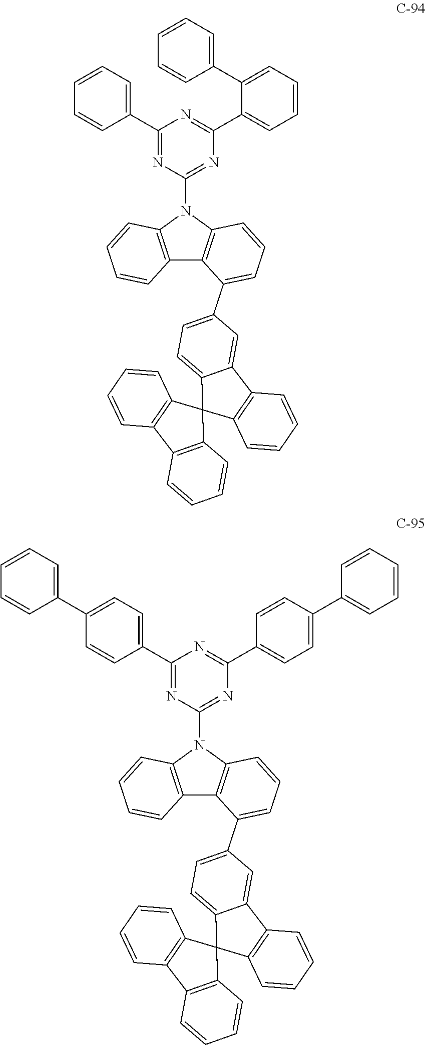 [SMILES: c1ccc(-c2nc(-c3ccccc3-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccc(-c5ccccc5)cc4)nc(-n4c5ccccc5c5c(-c6ccc7c(c6)-c6ccccc6C76c7ccccc7-c7ccccc76)cccc54)n3)cc2)cc1]

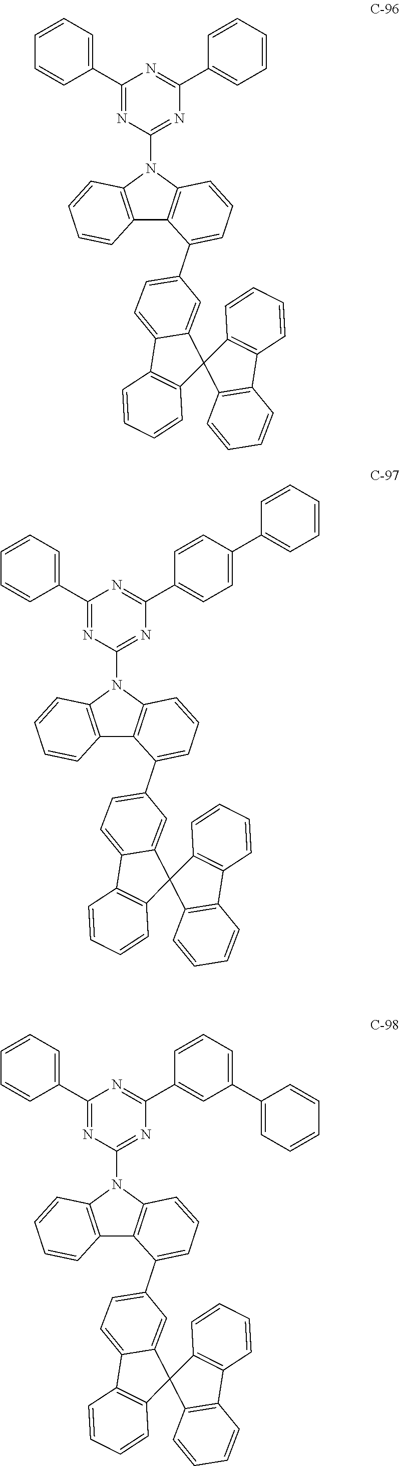 [SMILES: c1ccc(-c2nc(-c3ccccc3)nc(-n3c4ccccc4c4c(-c5ccc6c(c5)C5(c7ccccc7-c7ccccc75)c5ccccc5-6)cccc43)n2)cc1 ; c1ccc(-c2ccc(-c3nc(-c4ccccc4)nc(-n4c5ccccc5c5c(-c6ccc7c(c6)C6(c8ccccc8-c8ccccc86)c6ccccc6-7)cccc54)n3)cc2)cc1 ; c1ccc(-c2cccc(-c3nc(-c4ccccc4)nc(-n4c5ccccc5c5c(-c6ccc7c(c6)C6(c8ccccc8-c8ccccc86)c6ccccc6-7)cccc54)n3)c2)cc1]

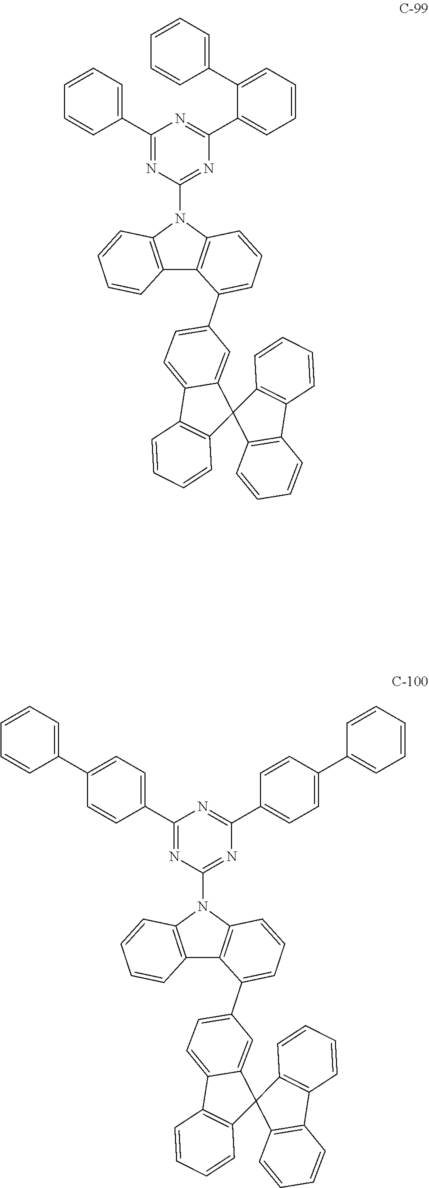 [SMILES: c1ccc(-c2nc(-c3ccccc3-c3ccccc3)nc(-n3c4ccccc4c4c(-c5ccc6c(c5)C5(c7ccccc7-c7ccccc75)c5ccccc5-6)cccc43)n2)cc1 ; c1ccc(-c2ccc(-c3nc(-c4ccc(-c5ccccc5)cc4)nc(-n4c5ccccc5c5c(-c6ccc7c(c6)C6(c8ccccc8-c8ccccc86)c6ccccc6-7)cccc54)n3)cc2)cc1]

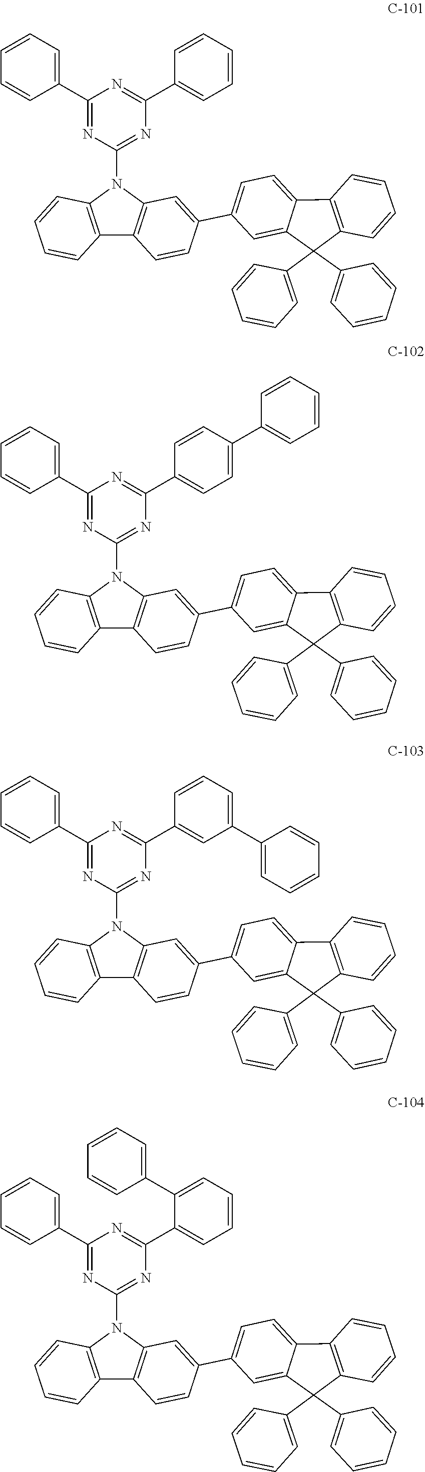 [SMILES: c1ccc(-c2nc(-c3ccccc3)nc(-n3c4ccccc4c4ccc(-c5ccc6c(c5)C(c5ccccc5)(c5ccccc5)c5ccccc5-6)cc43)n2)cc1 ; c1ccc(-c2ccc(-c3nc(-c4ccccc4)nc(-n4c5ccccc5c5ccc(-c6ccc7c(c6)C(c6ccccc6)(c6ccccc6)c6ccccc6-7)cc54)n3)cc2)cc1 ; c1ccc(-c2cccc(-c3nc(-c4ccccc4)nc(-n4c5ccccc5c5ccc(-c6ccc7c(c6)C(c6ccccc6)(c6ccccc6)c6ccccc6-7)cc54)n3)c2)cc1 ; c1ccc(-c2nc(-c3ccccc3-c3ccccc3)nc(-n3c4ccccc4c4ccc(-c5ccc6c(c5)C(c5ccccc5)(c5ccccc5)c5ccccc5-6)cc43)n2)cc1]

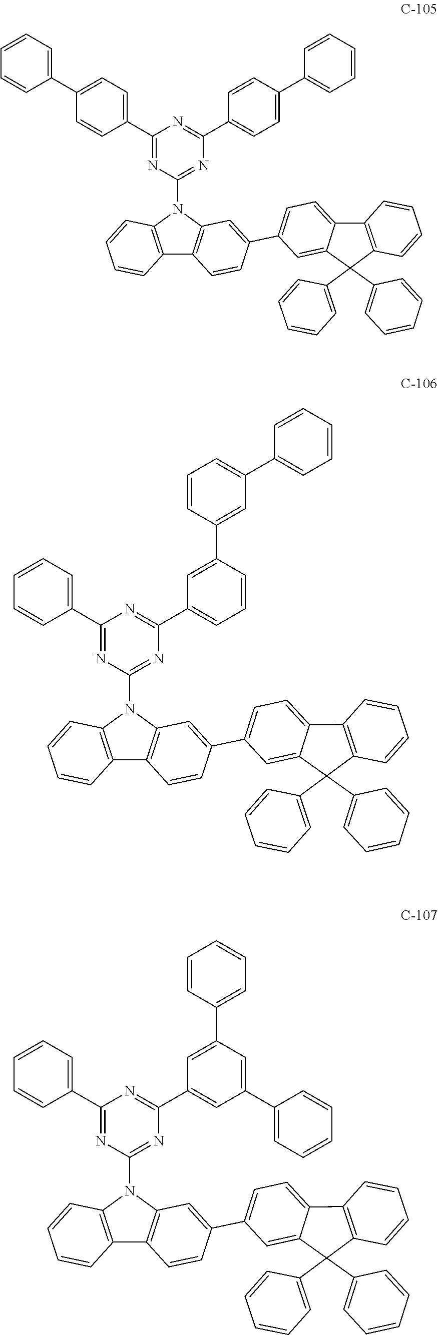 [SMILES: c1ccc(-c2ccc(-c3nc(-c4ccc(-c5ccccc5)cc4)nc(-n4c5ccccc5c5ccc(-c6ccc7c(c6)C(c6ccccc6)(c6ccccc6)c6ccccc6-7)cc54)n3)cc2)cc1 ; c1ccc(-c2cccc(-c3cccc(-c4nc(-c5ccccc5)nc(-n5c6ccccc6c6ccc(-c7ccc8c(c7)C(c7ccccc7)(c7ccccc7)c7ccccc7-8)cc65)n4)c3)c2)cc1 ; c1ccc(-c2cc(-c3ccccc3)cc(-c3nc(-c4ccccc4)nc(-n4c5ccccc5c5ccc(-c6ccc7c(c6)C(c6ccccc6)(c6ccccc6)c6ccccc6-7)cc54)n3)c2)cc1]

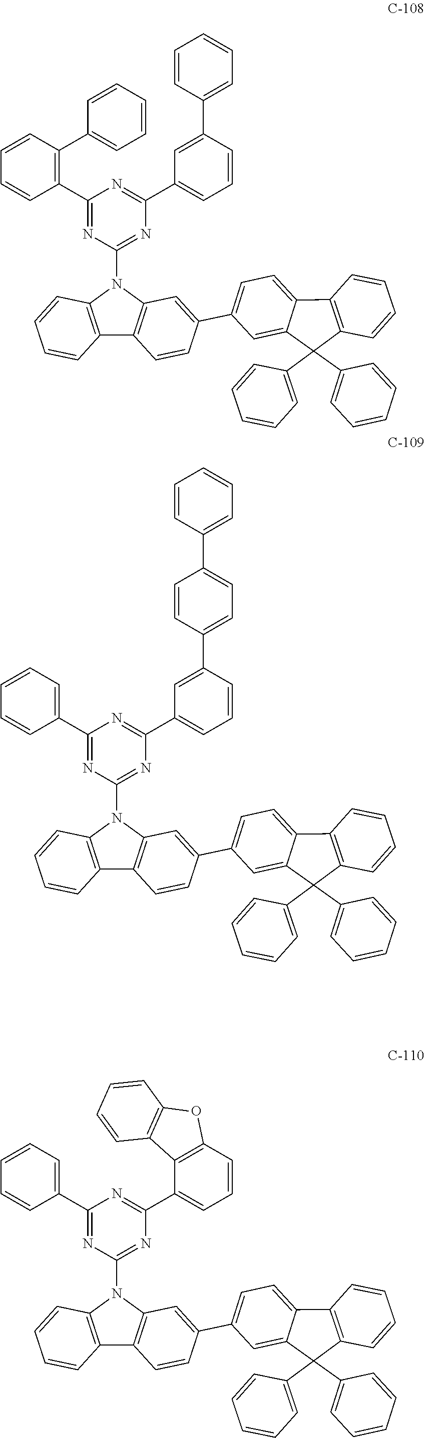 [SMILES: c1ccc(-c2cccc(-c3nc(-c4ccccc4-c4ccccc4)nc(-n4c5ccccc5c5ccc(-c6ccc7c(c6)C(c6ccccc6)(c6ccccc6)c6ccccc6-7)cc54)n3)c2)cc1 ; c1ccc(-c2ccc(-c3cccc(-c4nc(-c5ccccc5)nc(-n5c6ccccc6c6ccc(-c7ccc8c(c7)C(c7ccccc7)(c7ccccc7)c7ccccc7-8)cc65)n4)c3)cc2)cc1 ; c1ccc(-c2nc(-c3cccc4oc5ccccc5c34)nc(-n3c4ccccc4c4ccc(-c5ccc6c(c5)C(c5ccccc5)(c5ccccc5)c5ccccc5-6)cc43)n2)cc1]

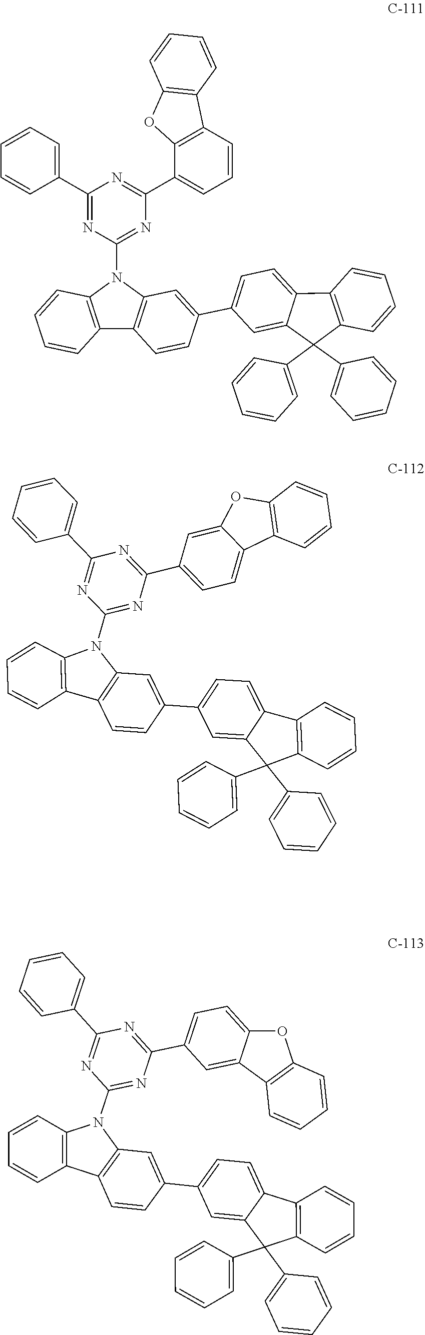 [SMILES: c1ccc(-c2nc(-c3cccc4c3oc3ccccc34)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccc4c(c3)oc3ccccc34)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccc4oc5ccccc5c4c3)nc(-n3c4ccccc4c4ccc(-c5ccc6c(c5)C(c5ccccc5)(c5ccccc5)c5ccccc5-6)cc43)n2)cc1]

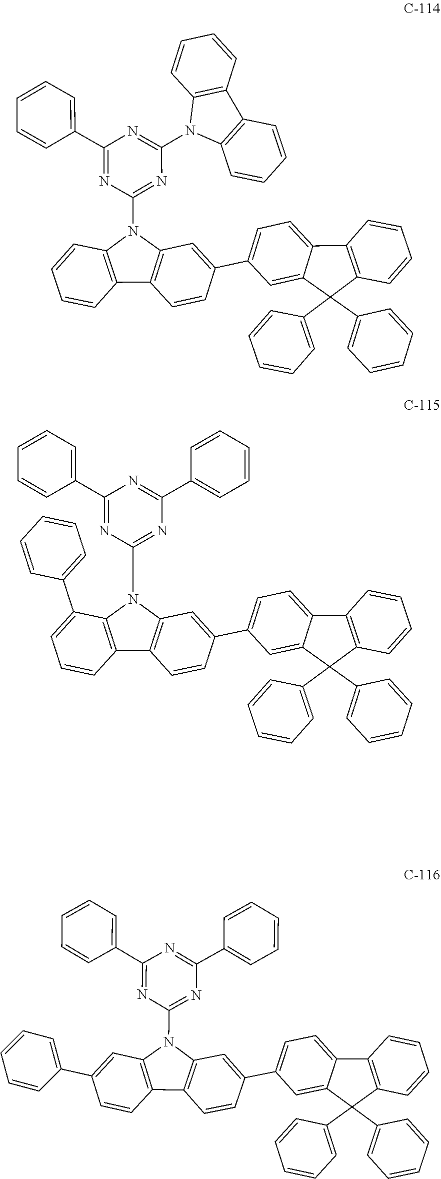 [SMILES: c1ccc(-c2nc(-n3c4ccccc4c4ccccc43)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccccc3)nc(-n3c4cc(-c5ccc6c(c5)C(c5ccccc5)(c5ccccc5)c5ccccc5-6)ccc4c4cccc(-c5ccccc5)c43)n2)cc1 ; c1ccc(-c2ccc3c4ccc(-c5ccc6c(c5)C(c5ccccc5)(c5ccccc5)c5ccccc5-6)cc4n(-c4nc(-c5ccccc5)nc(-c5ccccc5)n4)c3c2)cc1]

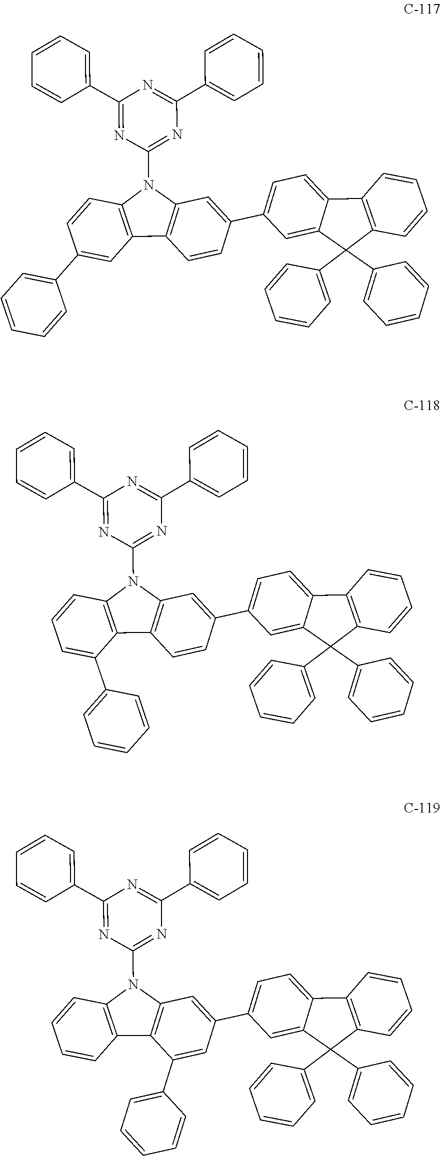 [SMILES: c1ccc(-c2ccc3c(c2)c2ccc(-c4ccc5c(c4)C(c4ccccc4)(c4ccccc4)c4ccccc4-5)cc2n3-c2nc(-c3ccccc3)nc(-c3ccccc3)n2)cc1 ; c1ccc(-c2nc(-c3ccccc3)nc(-n3c4cc(-c5ccc6c(c5)C(c5ccccc5)(c5ccccc5)c5ccccc5-6)ccc4c4c(-c5ccccc5)cccc43)n2)cc1 ; c1ccc(-c2nc(-c3ccccc3)nc(-n3c4ccccc4c4c(-c5ccccc5)cc(-c5ccc6c(c5)C(c5ccccc5)(c5ccccc5)c5ccccc5-6)cc43)n2)cc1]

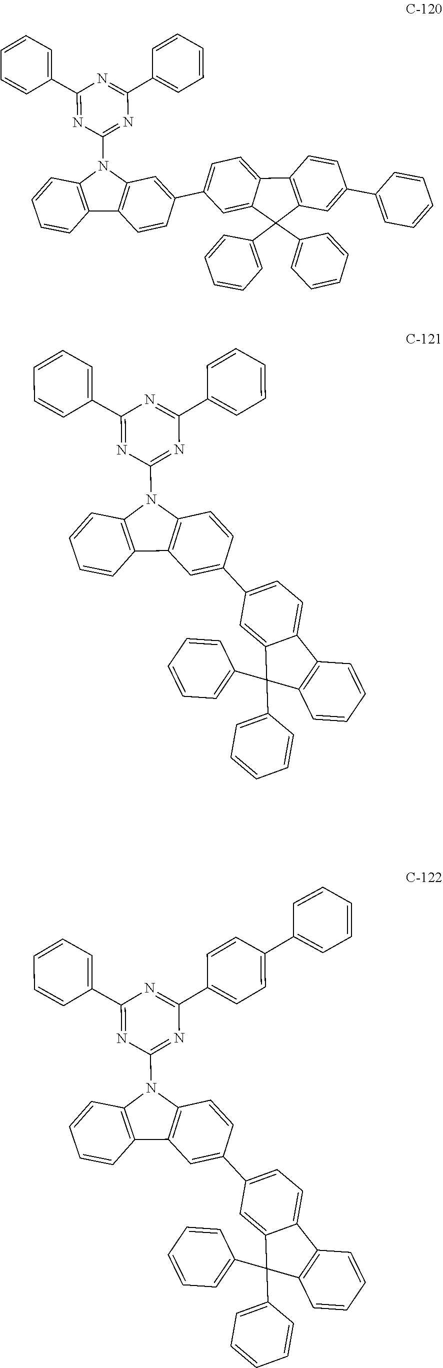 [SMILES: c1ccc(-c2ccc3c(c2)C(c2ccccc2)(c2ccccc2)c2cc(-c4ccc5c6ccccc6n(-c6nc(-c7ccccc7)nc(-c7ccccc7)n6)c5c4)ccc2-3)cc1 ; c1ccc(-c2nc(-c3ccccc3)nc(-n3c4ccccc4c4cc(-c5ccc6c(c5)C(c5ccccc5)(c5ccccc5)c5ccccc5-6)ccc43)n2)cc1 ; c1ccc(-c2ccc(-c3nc(-c4ccccc4)nc(-n4c5ccccc5c5cc(-c6ccc7c(c6)C(c6ccccc6)(c6ccccc6)c6ccccc6-7)ccc54)n3)cc2)cc1]

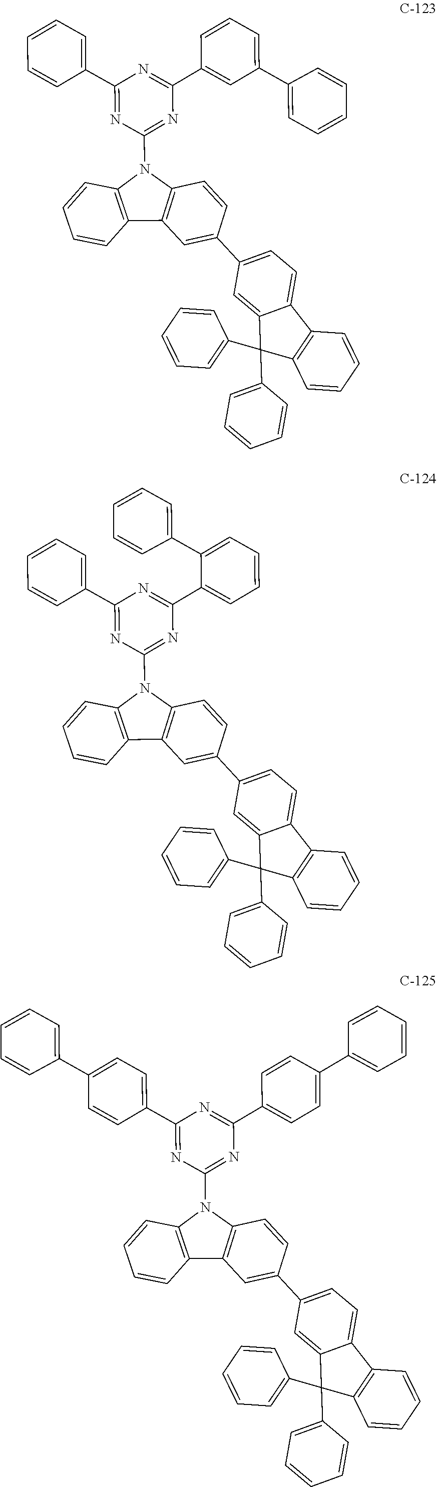 [SMILES: c1ccc(-c2cccc(-c3nc(-c4ccccc4)nc(-n4c5ccccc5c5cc(-c6ccc7c(c6)C(c6ccccc6)(c6ccccc6)c6ccccc6-7)ccc54)n3)c2)cc1 ; c1ccc(-c2nc(-c3ccccc3-c3ccccc3)nc(-n3c4ccccc4c4cc(-c5ccc6c(c5)C(c5ccccc5)(c5ccccc5)c5ccccc5-6)ccc43)n2)cc1 ; c1ccc(-c2ccc(-c3nc(-c4ccc(-c5ccccc5)cc4)nc(-n4c5ccccc5c5cc(-c6ccc7c(c6)C(c6ccccc6)(c6ccccc6)c6ccccc6-7)ccc54)n3)cc2)cc1]

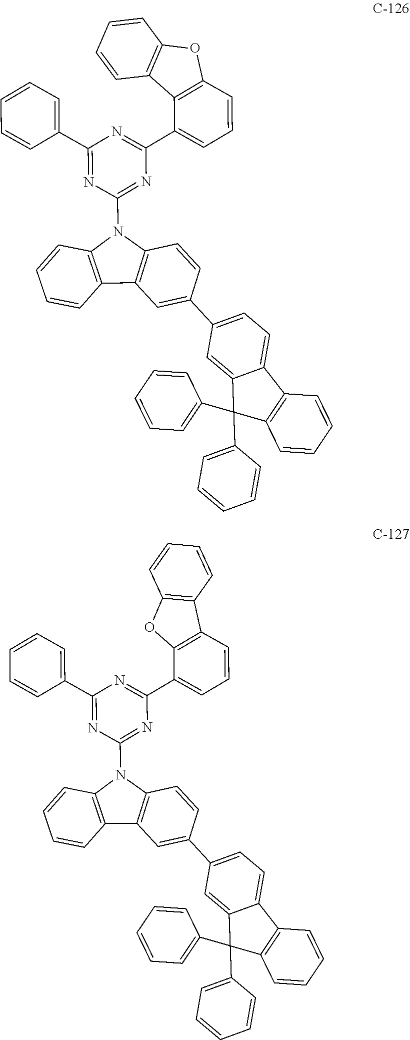

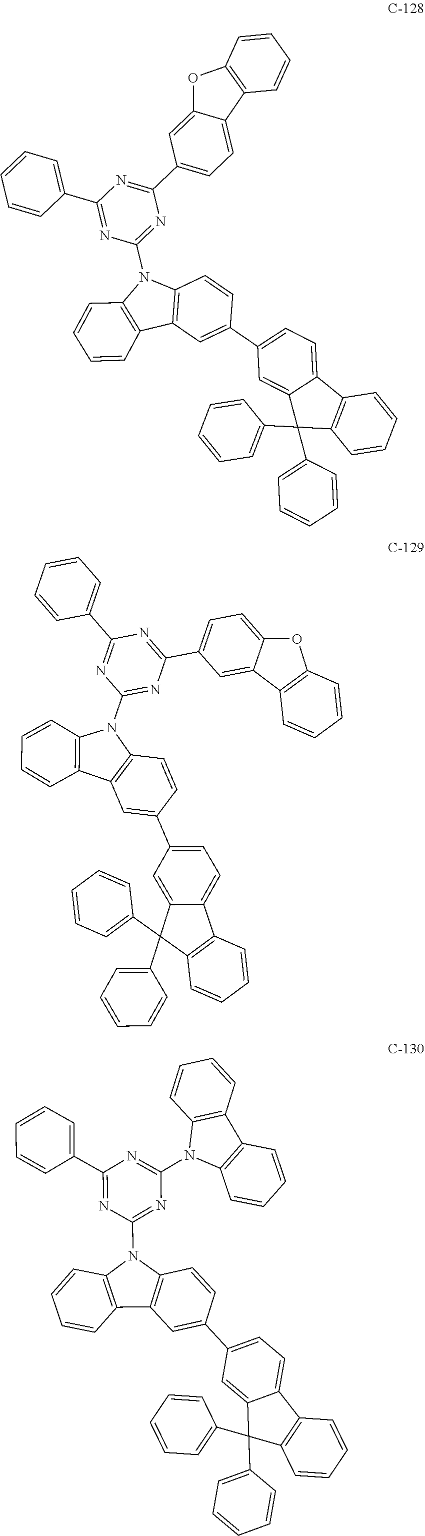 [SMILES: c1ccc(-c2nc(-c3ccc4c(c3)oc3ccccc34)nc(-n3c4ccccc4c4cc(-c5ccc6c(c5)C(c5ccccc5)(c5ccccc5)c5ccccc5-6)ccc43)n2)cc1 ; c1ccc(-c2nc(-c3ccc4oc5ccccc5c4c3)nc(-n3c4ccccc4c4cc(-c5ccc6c(c5)C(c5ccccc5)(c5ccccc5)c5ccccc5-6)ccc43)n2)cc1 ; c1ccc(-c2nc(-n3c4ccccc4c4ccccc43)nc(-n3c4ccccc4c4cc(-c5ccc6c(c5)C(c5ccccc5)(c5ccccc5)c5ccccc5-6)ccc43)n2)cc1]

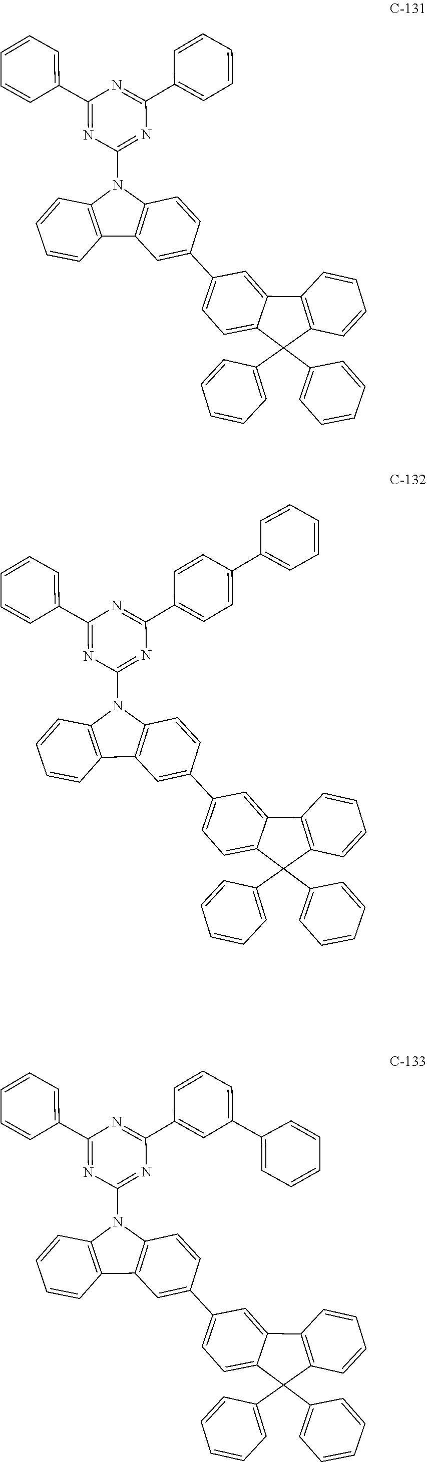 [SMILES: c1ccc(-c2nc(-c3ccccc3)nc(-n3c4ccccc4c4cc(-c5ccc6c(c5)-c5ccccc5C6(c5ccccc5)c5ccccc5)ccc43)n2)cc1 ; c1ccc(-c2ccc(-c3nc(-c4ccccc4)nc(-n4c5ccccc5c5cc(-c6ccc7c(c6)-c6ccccc6C7(c6ccccc6)c6ccccc6)ccc54)n3)cc2)cc1 ; c1ccc(-c2cccc(-c3nc(-c4ccccc4)nc(-n4c5ccccc5c5cc(-c6ccc7c(c6)-c6ccccc6C7(c6ccccc6)c6ccccc6)ccc54)n3)c2)cc1]

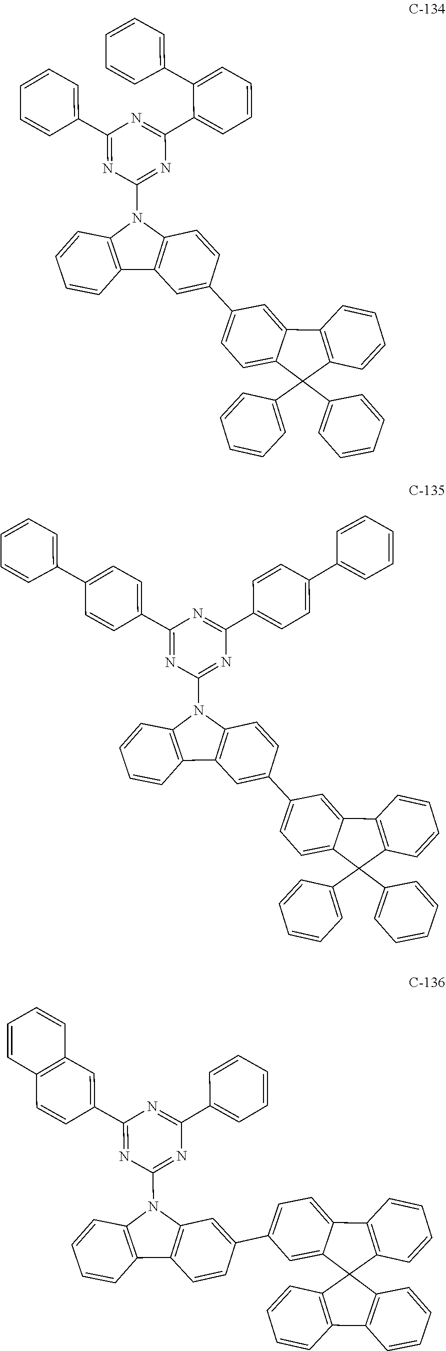 [SMILES: c1ccc(-c2nc(-c3ccccc3-c3ccccc3)nc(-n3c4ccccc4c4cc(-c5ccc6c(c5)-c5ccccc5C6(c5ccccc5)c5ccccc5)ccc43)n2)cc1 ; c1ccc(-c2ccc(-c3nc(-c4ccc(-c5ccccc5)cc4)nc(-n4c5ccccc5c5cc(-c6ccc7c(c6)-c6ccccc6C7(c6ccccc6)c6ccccc6)ccc54)n3)cc2)cc1 ; c1ccc(-c2nc(-c3ccc4ccccc4c3)nc(-n3c4ccccc4c4ccc(-c5ccc6c(c5)C5(c7ccccc7-c7ccccc75)c5ccccc5-6)cc43)n2)cc1]

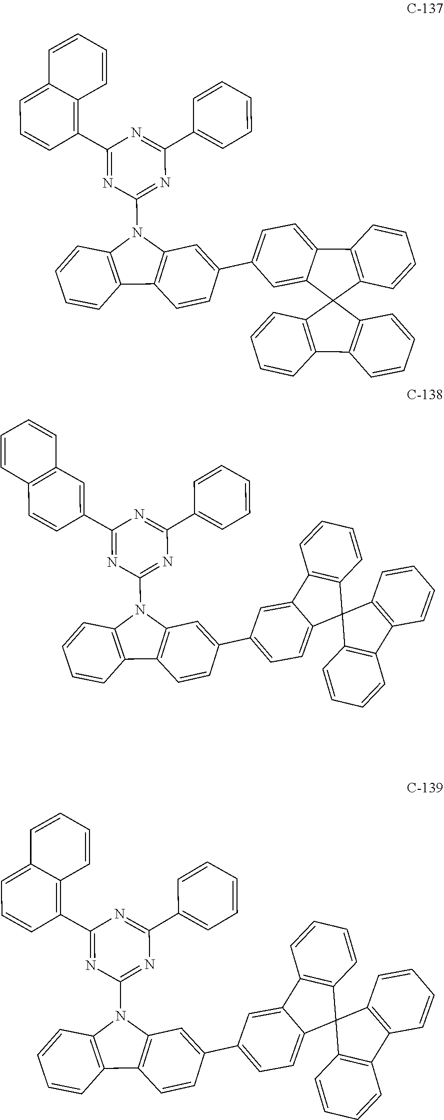 [SMILES: c1ccc(-c2nc(-c3cccc4ccccc34)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccc4ccccc4c3)nc(-n3c4ccccc4c4ccc(-c5ccc6c(c5)-c5ccccc5C65c6ccccc6-c6ccccc65)cc43)n2)cc1 ; c1ccc(-c2nc(-c3cccc4ccccc34)nc(-n3c4ccccc4c4ccc(-c5ccc6c(c5)-c5ccccc5C65c6ccccc6-c6ccccc65)cc43)n2)cc1]

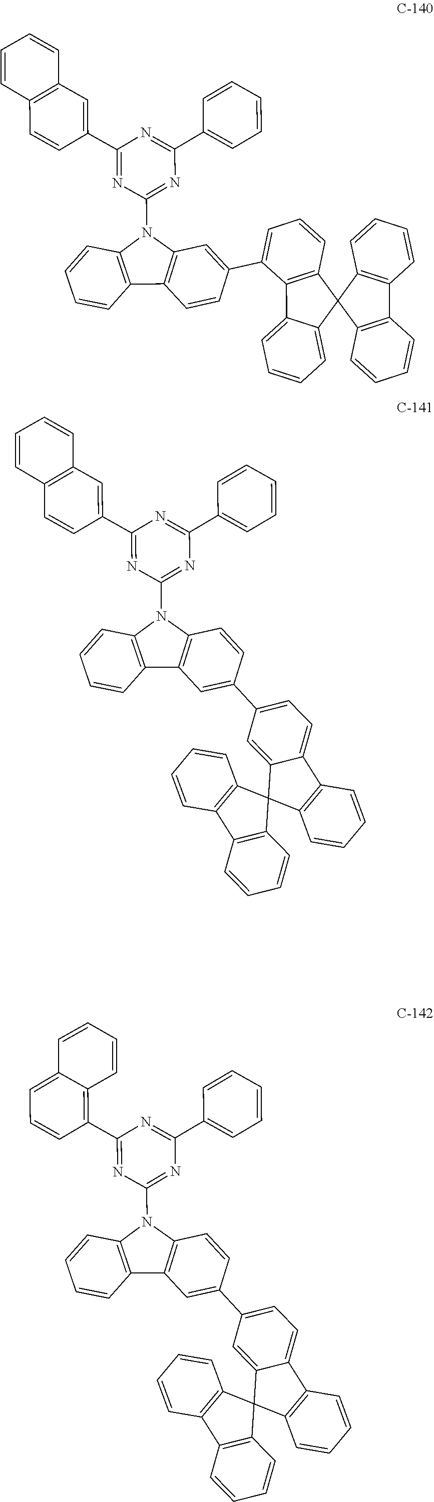 [SMILES: c1ccc(-c2nc(-c3ccc4ccccc4c3)nc(-n3c4ccccc4c4ccc(-c5cccc6c5-c5ccccc5C65c6ccccc6-c6ccccc65)cc43)n2)cc1 ; c1ccc(-c2nc(-c3ccc4ccccc4c3)nc(-n3c4ccccc4c4cc(-c5ccc6c(c5)C5(c7ccccc7-c7ccccc75)c5ccccc5-6)ccc43)n2)cc1 ; c1ccc(-c2nc(-c3cccc4ccccc34)nc(-n3c4ccccc4c4cc(-c5ccc6c(c5)C5(c7ccccc7-c7ccccc75)c5ccccc5-6)ccc43)n2)cc1]

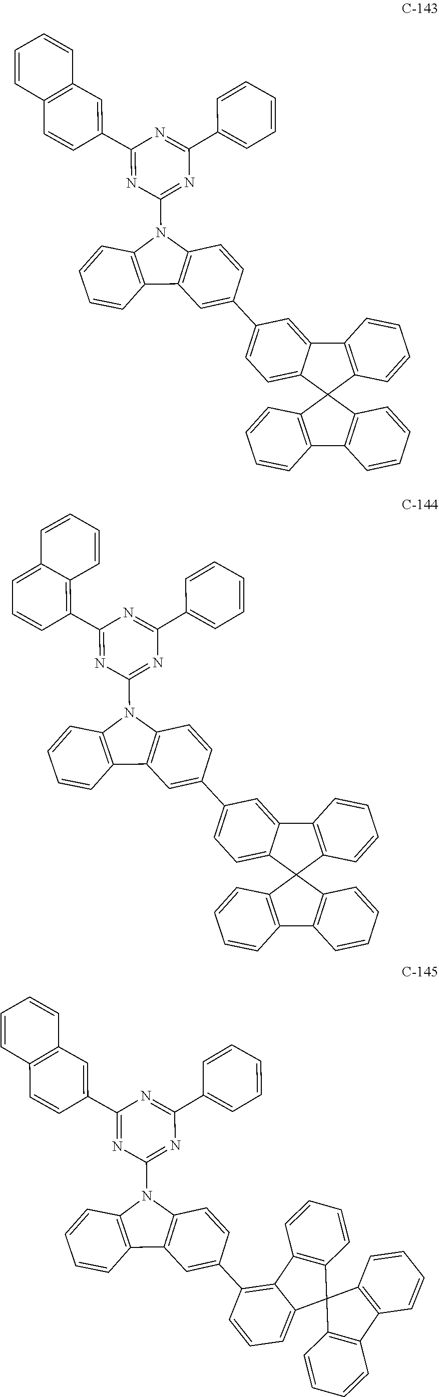 [SMILES: c1ccc(-c2nc(-c3ccc4ccccc4c3)nc(-n3c4ccccc4c4cc(-c5ccc6c(c5)-c5ccccc5C65c6ccccc6-c6ccccc65)ccc43)n2)cc1 ; c1ccc(-c2nc(-c3cccc4ccccc34)nc(-n3c4ccccc4c4cc(-c5ccc6c(c5)-c5ccccc5C65c6ccccc6-c6ccccc65)ccc43)n2)cc1 ; c1ccc(-c2nc(-c3ccc4ccccc4c3)nc(-n3c4ccccc4c4cc(-c5cccc6c5-c5ccccc5C65c6ccccc6-c6ccccc65)ccc43)n2)cc1]

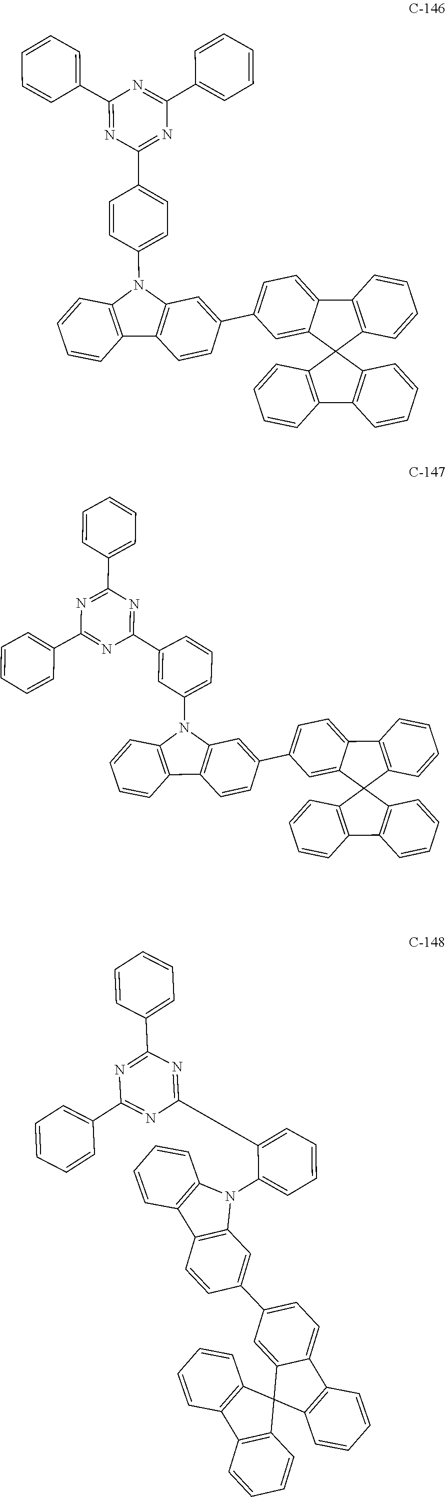 [SMILES: c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)cc3)n2)cc1 ; c1ccc(-c2nc(-c3ccccc3)nc(-c3cccc(-n4c5ccccc5c5ccc(-c6ccc7c(c6)C6(c8ccccc8-c8ccccc86)c6ccccc6-7)cc54)c3)n2)cc1 ; c1ccc(-c2nc(-c3ccccc3)nc(-c3ccccc3-n3c4ccccc4c4ccc(-c5ccc6c(c5)C5(c7ccccc7-c7ccccc75)c5ccccc5-6)cc43)n2)cc1]

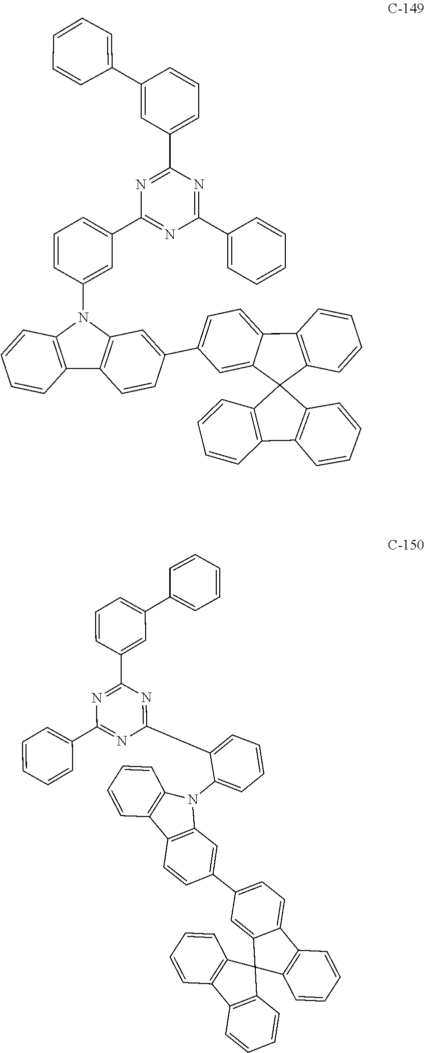 [SMILES: c1ccc(-c2cccc(-c3nc(-c4ccccc4)nc(-c4cccc(-n5c6ccccc6c6ccc(-c7ccc8c(c7)C7(c9ccccc9-c9ccccc97)c7ccccc7-8)cc65)c4)n3)c2)cc1 ; c1ccc(-c2cccc(-c3nc(-c4ccccc4)nc(-c4ccccc4-n4c5ccccc5c5ccc(-c6ccc7c(c6)C6(c8ccccc8-c8ccccc86)c6ccccc6-7)cc54)n3)c2)cc1]

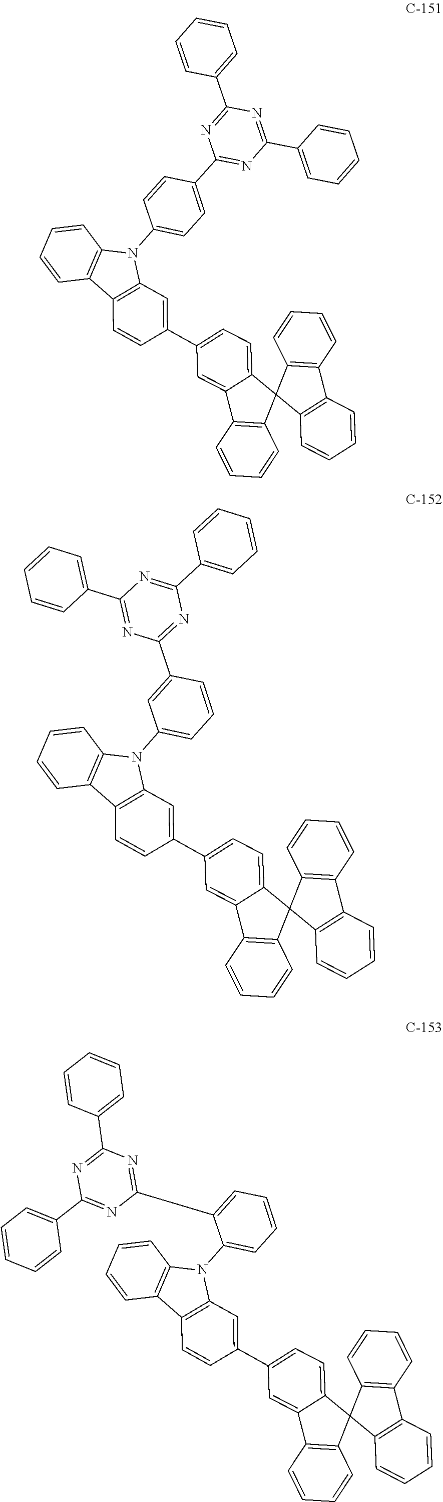

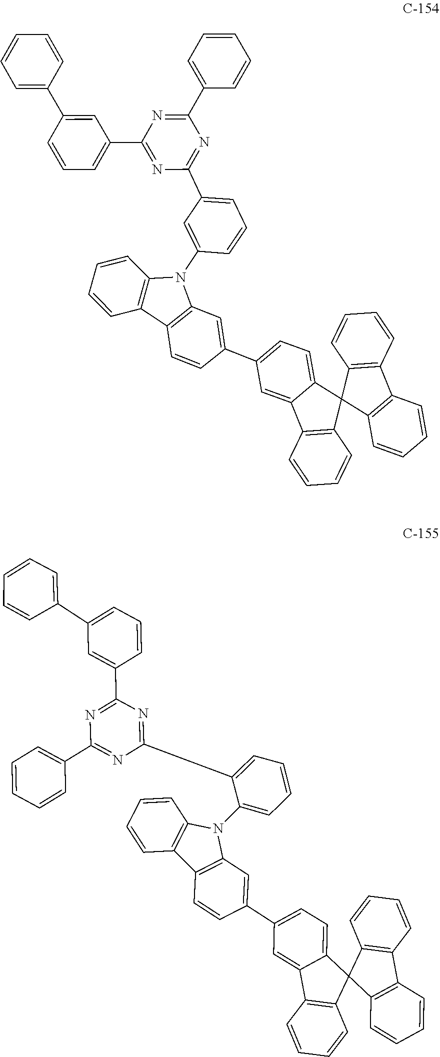 [SMILES: c1ccc(-c2cccc(-c3nc(-c4ccccc4)nc(-c4cccc(-n5c6ccccc6c6ccc(-c7ccc8c(c7)-c7ccccc7C87c8ccccc8-c8ccccc87)cc65)c4)n3)c2)cc1 ; c1ccc(-c2cccc(-c3nc(-c4ccccc4)nc(-c4ccccc4-n4c5ccccc5c5ccc(-c6ccc7c(c6)-c6ccccc6C76c7ccccc7-c7ccccc76)cc54)n3)c2)cc1]

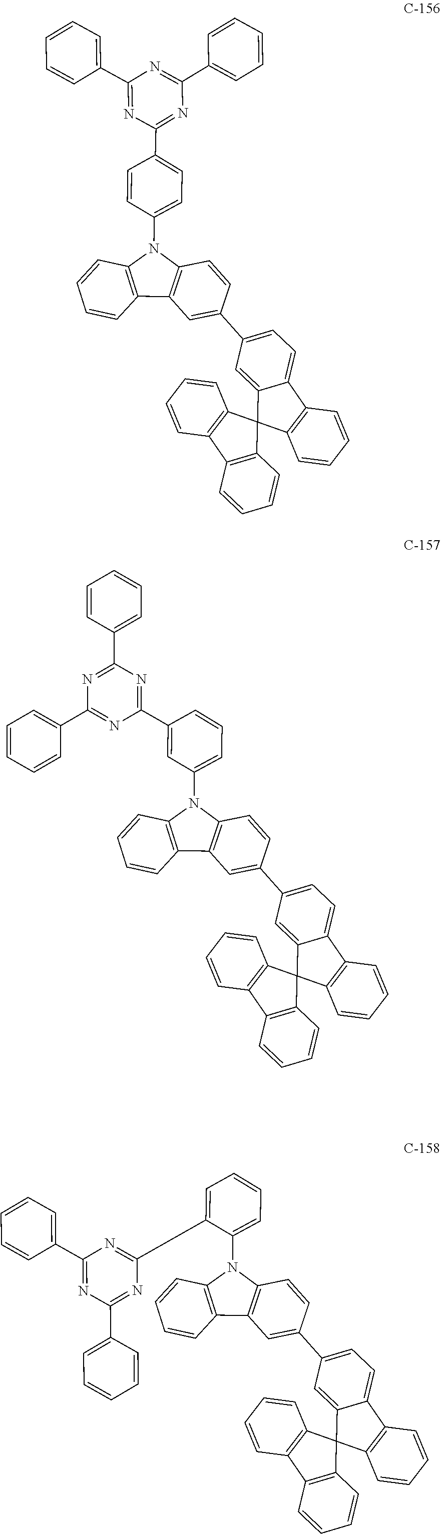 [SMILES: c1ccc(-c2nc(-c3ccccc3)nc(-c3ccc(-n4c5ccccc5c5cc(-c6ccc7c(c6)C6(c8ccccc8-c8ccccc86)c6ccccc6-7)ccc54)cc3)n2)cc1 ; c1ccc(-c2nc(-c3ccccc3)nc(-c3cccc(-n4c5ccccc5c5cc(-c6ccc7c(c6)C6(c8ccccc8-c8ccccc86)c6ccccc6-7)ccc54)c3)n2)cc1 ; c1ccc(-c2nc(-c3ccccc3)nc(-c3ccccc3-n3c4ccccc4c4cc(-c5ccc6c(c5)C5(c7ccccc7-c7ccccc75)c5ccccc5-6)ccc43)n2)cc1]

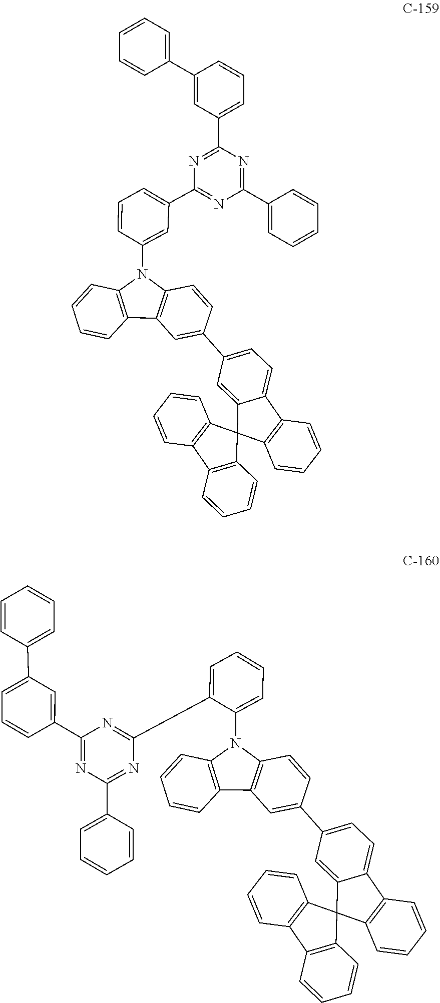 [SMILES: c1ccc(-c2cccc(-c3nc(-c4ccccc4)nc(-c4cccc(-n5c6ccccc6c6cc(-c7ccc8c(c7)C7(c9ccccc9-c9ccccc97)c7ccccc7-8)ccc65)c4)n3)c2)cc1 ; c1ccc(-c2cccc(-c3nc(-c4ccccc4)nc(-c4ccccc4-n4c5ccccc5c5cc(-c6ccc7c(c6)C6(c8ccccc8-c8ccccc86)c6ccccc6-7)ccc54)n3)c2)cc1]

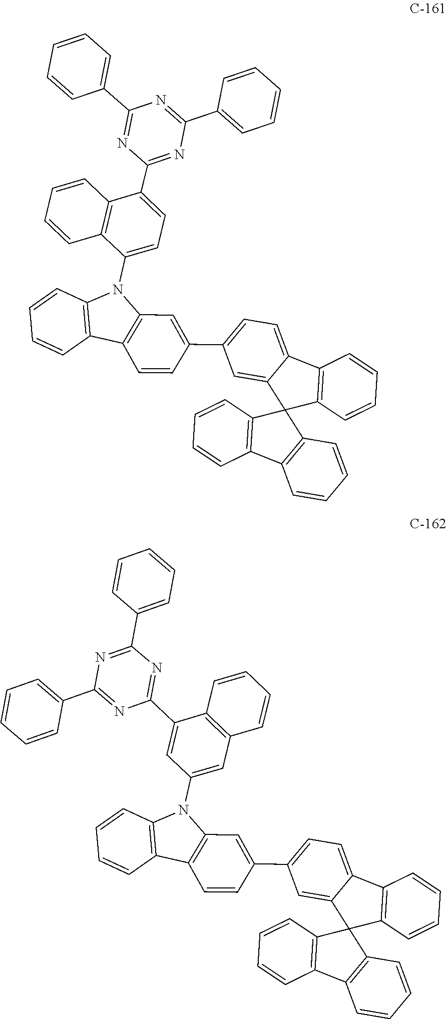 [SMILES: c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)c4ccccc34)n2)cc1 ; c1ccc(-c2nc(-c3ccccc3)nc(-c3cc(-n4c5ccccc5c5ccc(-c6ccc7c(c6)C6(c8ccccc8-c8ccccc86)c6ccccc6-7)cc54)cc4ccccc34)n2)cc1]

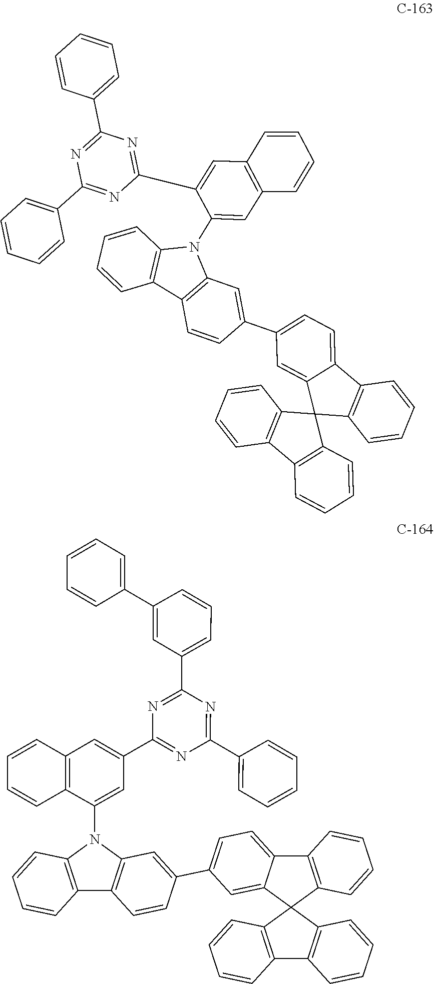 [SMILES: c1ccc(-c2nc(-c3ccccc3)nc(-c3cc4ccccc4cc3-n3c4ccccc4c4ccc(-c5ccc6c(c5)C5(c7ccccc7-c7ccccc75)c5ccccc5-6)cc43)n2)cc1 ; c1ccc(-c2cccc(-c3nc(-c4ccccc4)nc(-c4cc(-n5c6ccccc6c6ccc(-c7ccc8c(c7)C7(c9ccccc9-c9ccccc97)c7ccccc7-8)cc65)c5ccccc5c4)n3)c2)cc1]

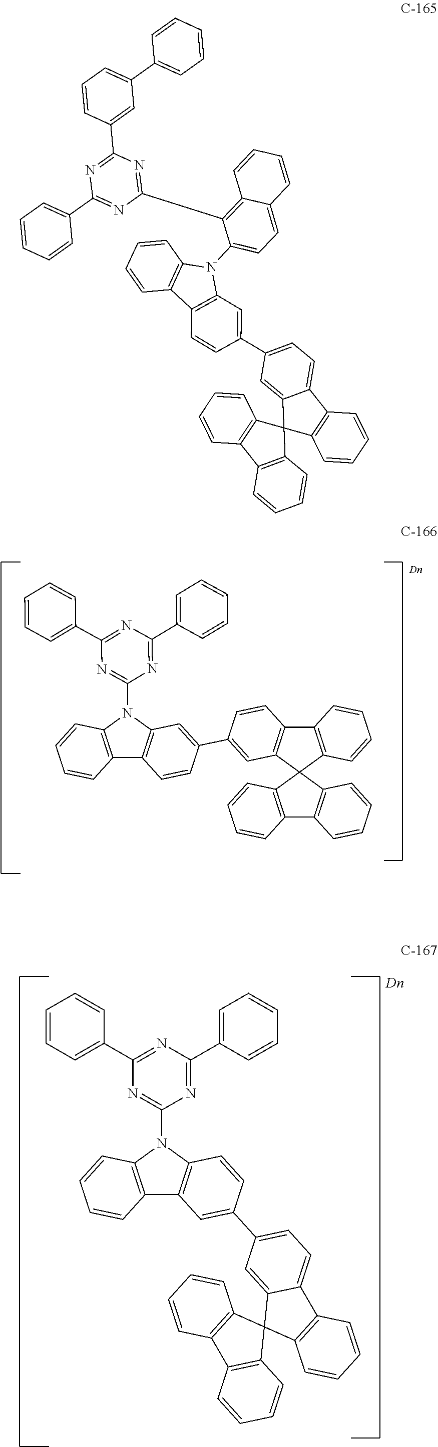

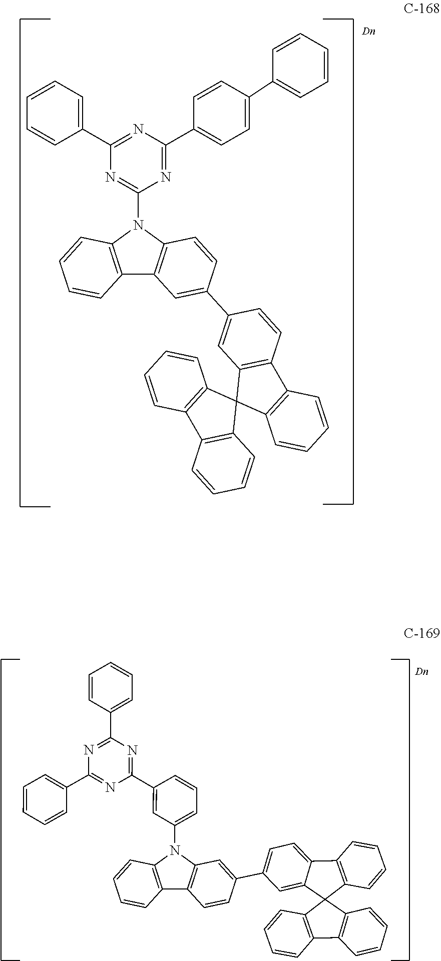

In the compounds above, Dn means that n number of hydrogens is replaced with deuterium, wherein n represents an integer of 1 or more and the upper limit of n is determined by the number of hydrogens that can be substituted in each compound.

The host compound represented by Formula 1 according to the present disclosure may be prepared by referring to the following reaction schemes 1 to 4, but is not limited thereto and may be produced by a synthetic method known to a person skilled in the art.

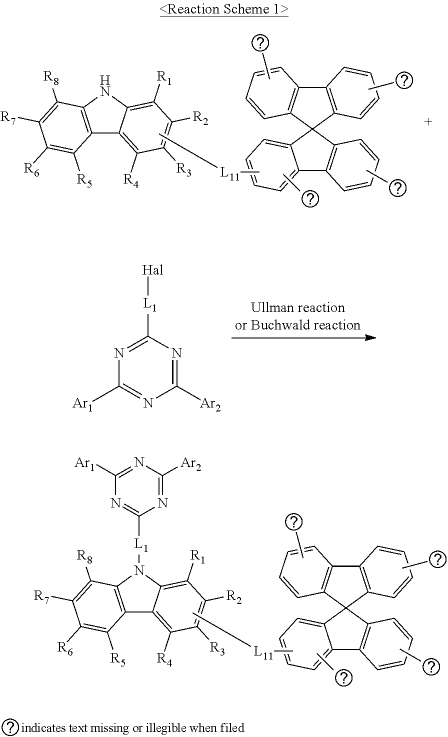

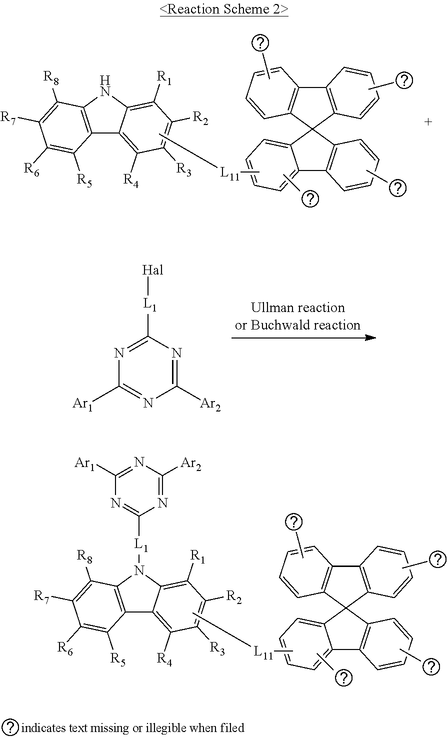

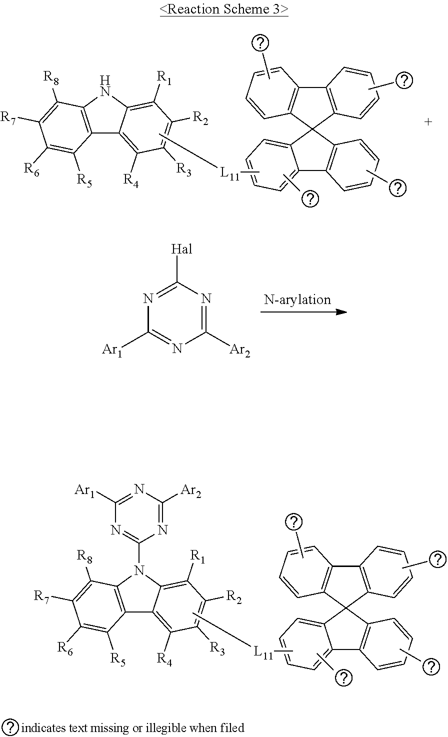

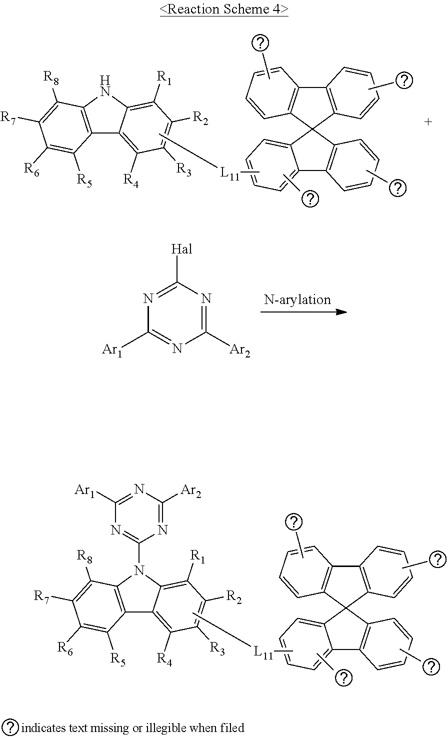

In reaction schemes 1 to 4, the definition of each of the substituents is as defined in Formula 1, and Hal represent a halogen atom.

As described above, exemplary synthesis examples of the compounds represented by Formula 1 according to the present disclosure are described, but they are based on Buchwald-Hartwig cross coupling reaction, N-arylation reaction, H-mont-mediated etherification reaction, Miyaura borylation reaction, Suzuki cross-coupling reaction, Intramolecular acid-induced cyclization reaction, Pd(II)-catalyzed oxidative cyclization reaction, Grignard reaction, Heck reaction, Cyclic Dehydration reaction, SN₁ substitution reaction, SN₂ substitution reaction, and Phosphine-mediated reductive cyclization reaction, etc. It will be understood by one skilled in the art that the above reaction proceeds even if other substituents defined in the Formula 1 other than the substituents described in the specific synthesis examples are bonded.

The second host compound as another host material according to one embodiment may be represented by the following Formula 2.

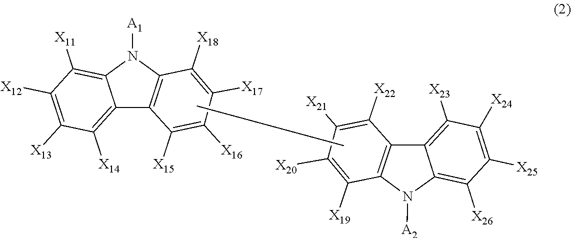

In Formula 2,
A₁ and A₂ each independently represent, (C6-C30)aryl unsubstituted or substituted with deuterium, a substituted or unsubstituted dibenzofuranyl, a substituted or unsubstituted dibenzothiophenyl, or a substituted or unsubstituted carbazolyl;
X₁₁ to X₂₆ each independently represent hydrogen, deuterium, a substituted or unsubstituted (C6-C30)aryl, or a substituted or unsubstituted (3- to 30-membered)heteroaryl; or may be linked to the adjacent substituents to form a ring(s); and
any one of X₁₅ to X₁₈ is linked to any one of X₁₉ to X₂₂.

In one embodiment, A₁ and A₂ each independently may be (C6-C30)aryl unsubstituted or substituted with deuterium, a substituted or unsubstituted dibenzofuranyl, a substituted or unsubstituted dibenzothiophenyl, or a substituted or unsubstituted carbazolyl, preferably (C6-C25)aryl unsubstituted or substituted with deuterium, dibenzofuranyl unsubstituted or substituted with deuterium or (C6-C30)aryl, dibenzothiophenyl unsubstituted or substituted with deuterium or (C6-C30)aryl, or carbazolyl unsubstituted or substituted with deuterium or (C6-C30)aryl, more preferably (C6-C18)aryl unsubstituted or substituted with deuterium, dibenzofuranyl unsubstituted or substituted with deuterium or (C6-C25)aryl, dibenzothiophenyl unsubstituted or substituted with deuterium or (C6-C25)aryl, or carbazolyl unsubstituted or substituted with deuterium or (C6-C25)aryl. For example, A₁ and A₂ each independently may be phenyl unsubstituted or substituted with deuterium, biphenyl unsubstituted or substituted with deuterium, terphenyl unsubstituted or substituted with deuterium, naphthyl unsubstituted or substituted with deuterium, fluorenyl unsubstituted or substituted with deuterium, benzofluorenyl unsubstituted or substituted with deuterium, triphenylenyl unsubstituted or substituted with deuterium, fluoranthenyl unsubstituted or substituted with deuterium, phenanthrenyl unsubstituted or substituted with deuterium, dibenzofuranyl unsubstituted or substituted with deuterium, carbazolyl unsubstituted or substituted with deuterium, dibenzothiophenyl unsubstituted or substituted with deuterium, or a combination thereof. For example, A₁ and A₂ each independently may be phenyl unsubstituted or substituted with deuterium, biphenyl unsubstituted or substituted with deuterium, terphenyl unsubstituted or substituted with deuterium, naphthyl unsubstituted or substituted with deuterium, phenylnaphthyl unsubstituted or substituted with deuterium, naphthylphenyl unsubstituted or substituted with deuterium, isochrysenyl unsubstituted or substituted with deuterium or phenyl, dibenzofuranyl unsubstituted or substituted with deuterium or phenyl, dibenzothiophenyl unsubstituted or substituted with deuterium or phenyl, or carbazolyl unsubstituted or substituted with at least one of deuterium, phenyl and naphthyl.

In one embodiment, the deuterium substitution rate of X₁₁ to X₂₆ in Formula 2 may be 25% to 100%. In one embodiment, at least four of X₁₁ to X₂₆ may be deuterium, at least one of X₁₁, X₁₈, X₁₉, and X₂₆, preferably two, more preferably three, and even more preferably four may be deuterium.

According to one embodiment, in one compound represented by Formula 2, the deuterium substitution rate may be 40% to 100%, preferably 50% to 100%, more preferably 60% to 100%, and even more preferably 75% to 100%. When deuterated with a number equal to or higher than the lower limit, the bond dissociation energy according to deuteration increases, thereby increasing the stability of the compound. When such a compound is used in an organic electroluminescent device, improved lifespan characteristics may be exhibited.

The second host compound of Formula 2 substituted with the deuterium substitution rate above can increase the stability of the compound by increasing the bond dissociation energy due to deuteration, and an organic electroluminescent device comprising the compound can exhibit improved lifespan characteristics.

The Formula 2 according to one embodiment may be represented by any one of the following formulas 2-1 to 2-8.

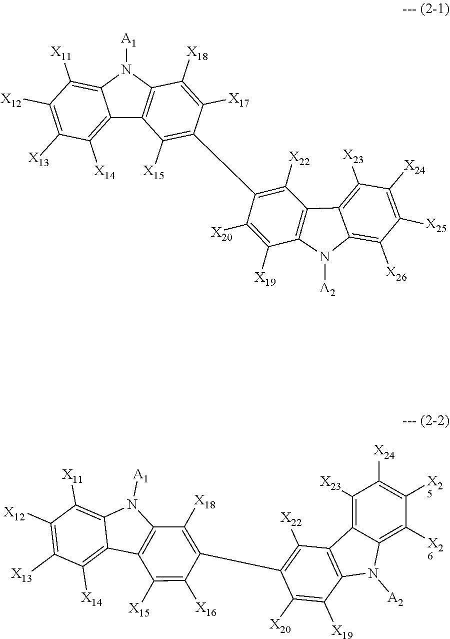

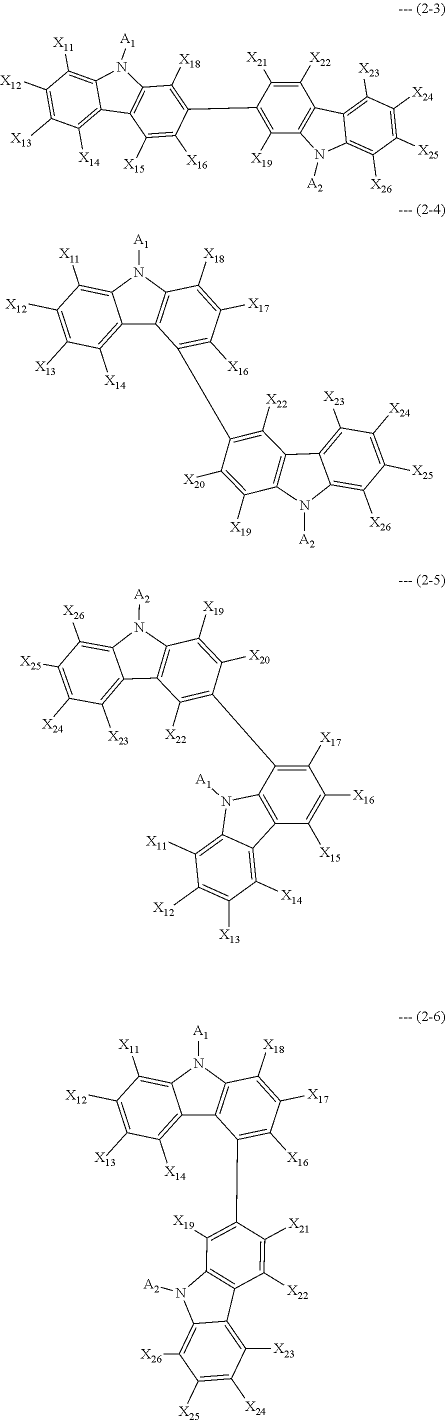

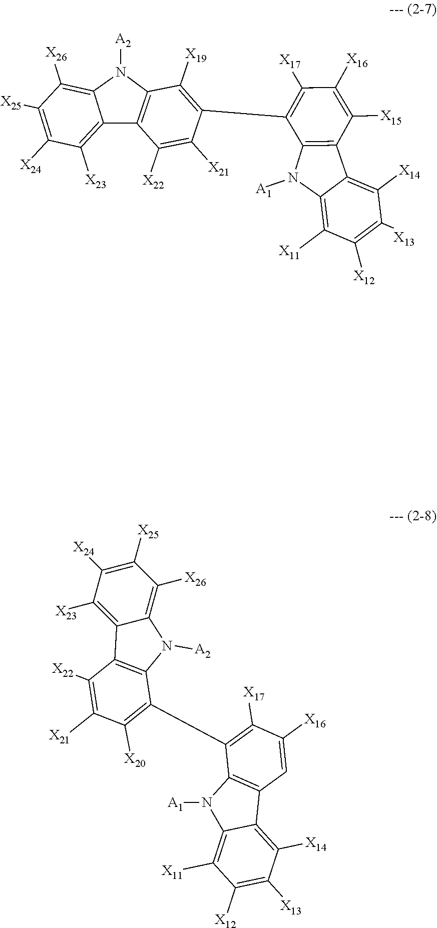

In formulas 2-1 to 2-8,
A₁, A₂ and X₁₁ to X₂₆ are as defined in Formula 2.

According to one embodiment, the compound represented by Formula 2 may be more specifically illustrated by the following compounds, but is not limited thereto.

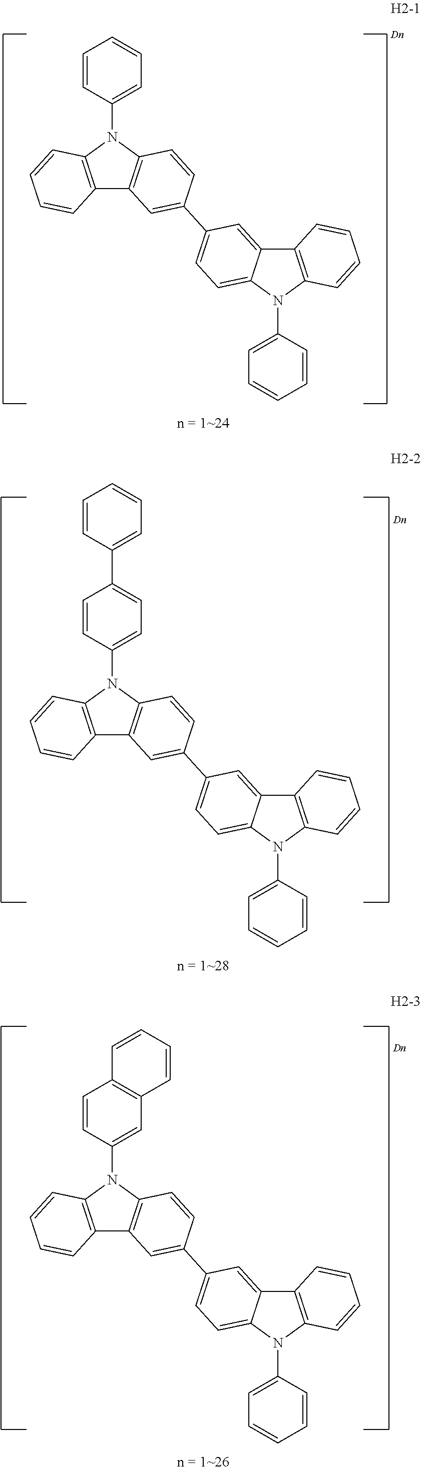

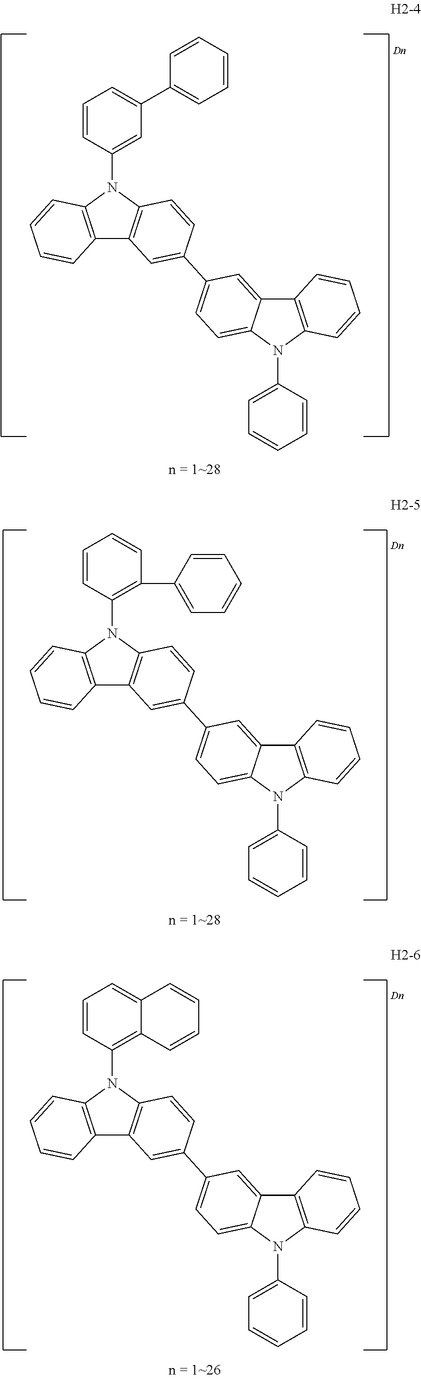

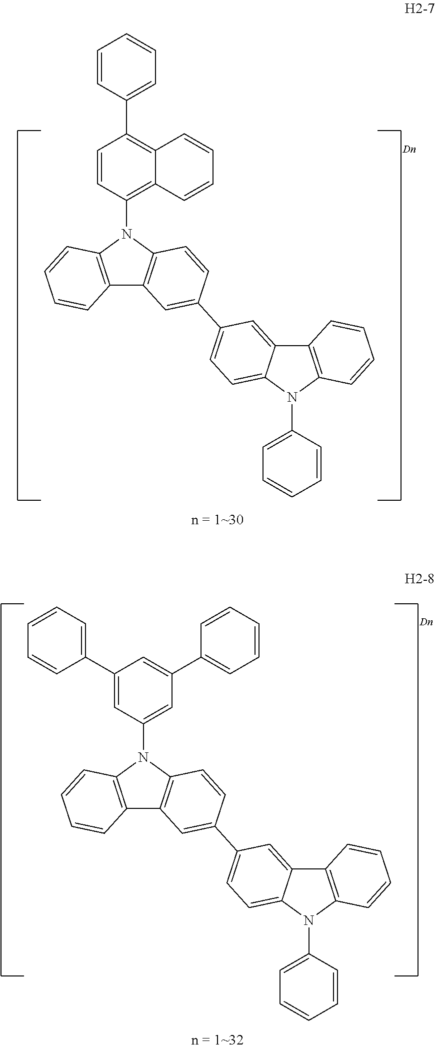

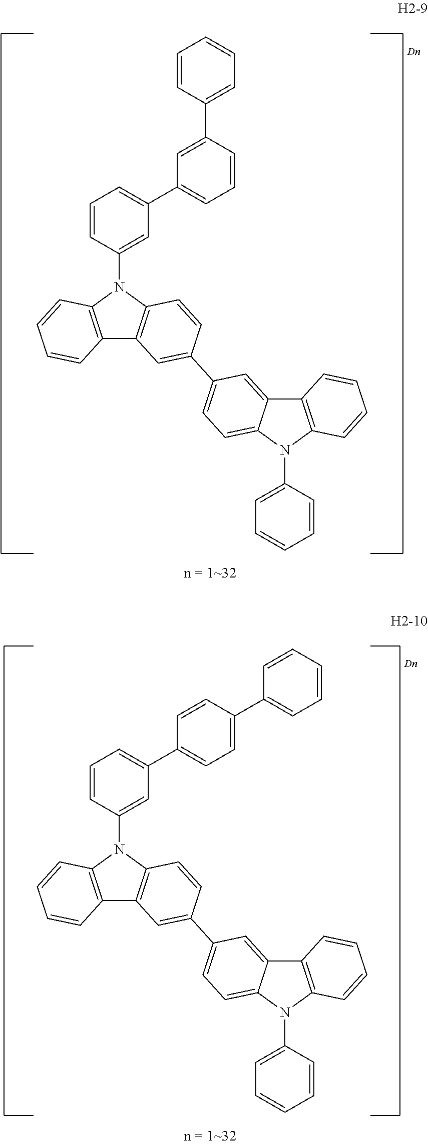

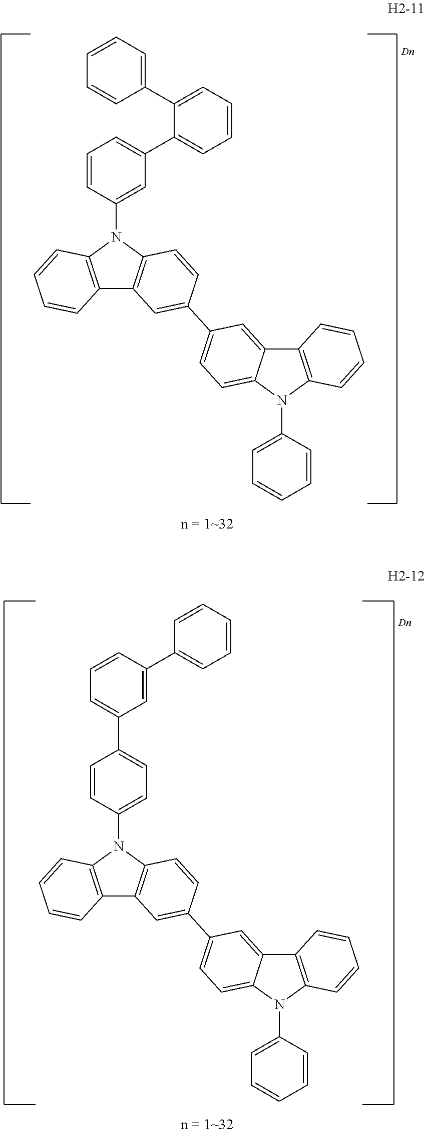

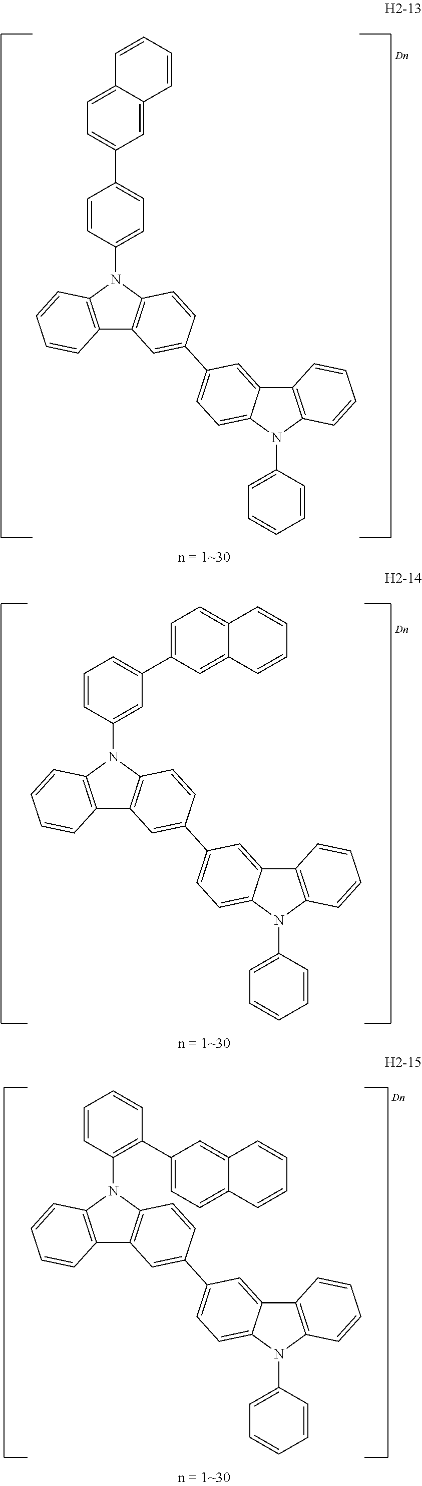

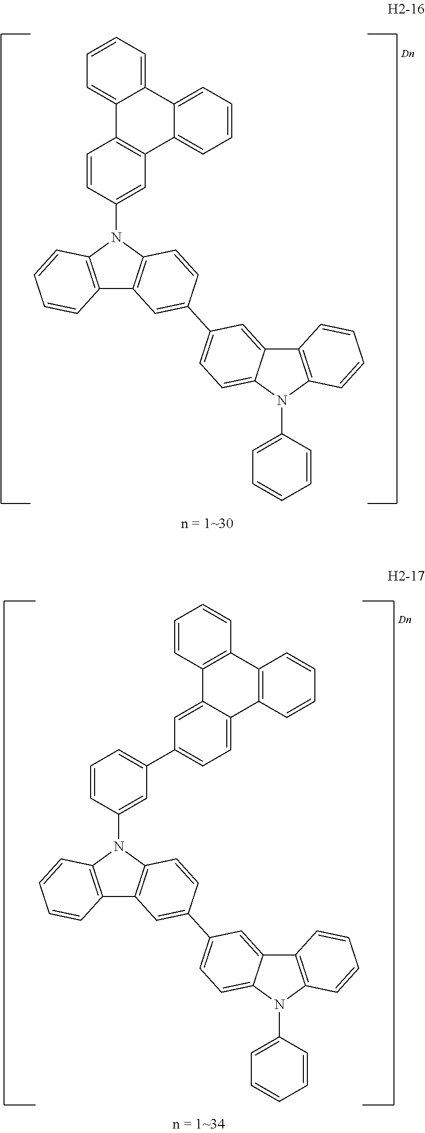

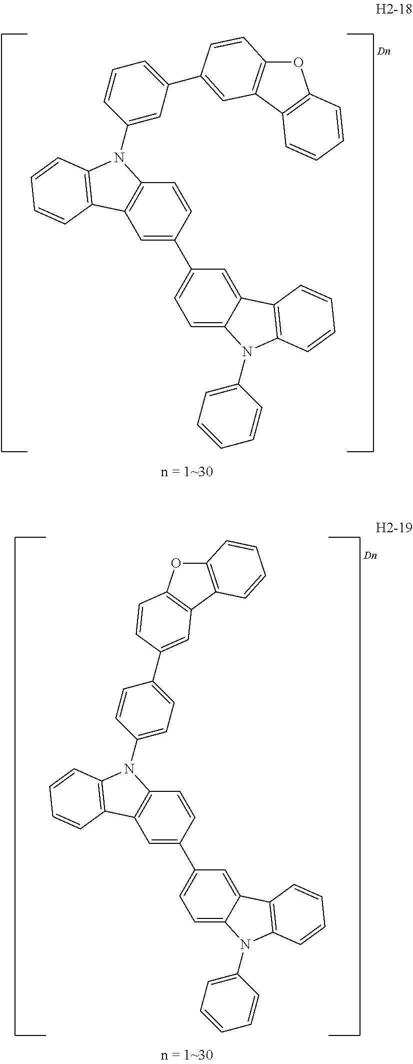

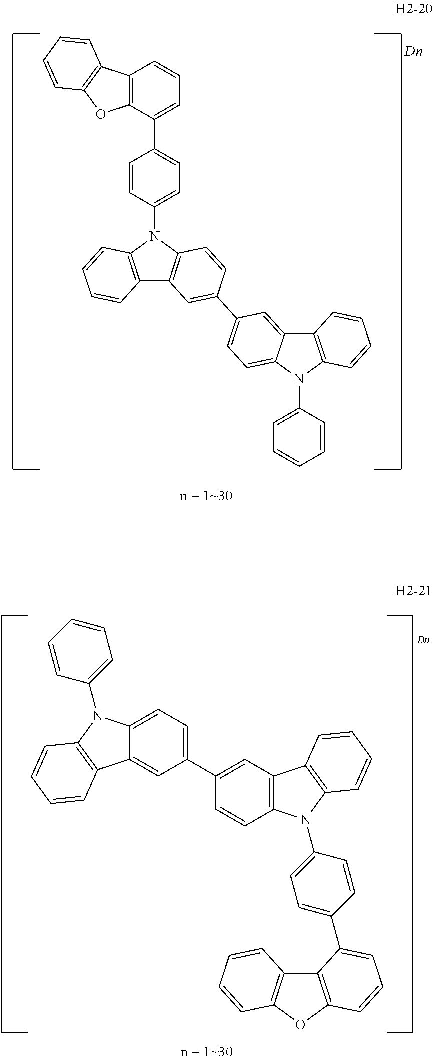

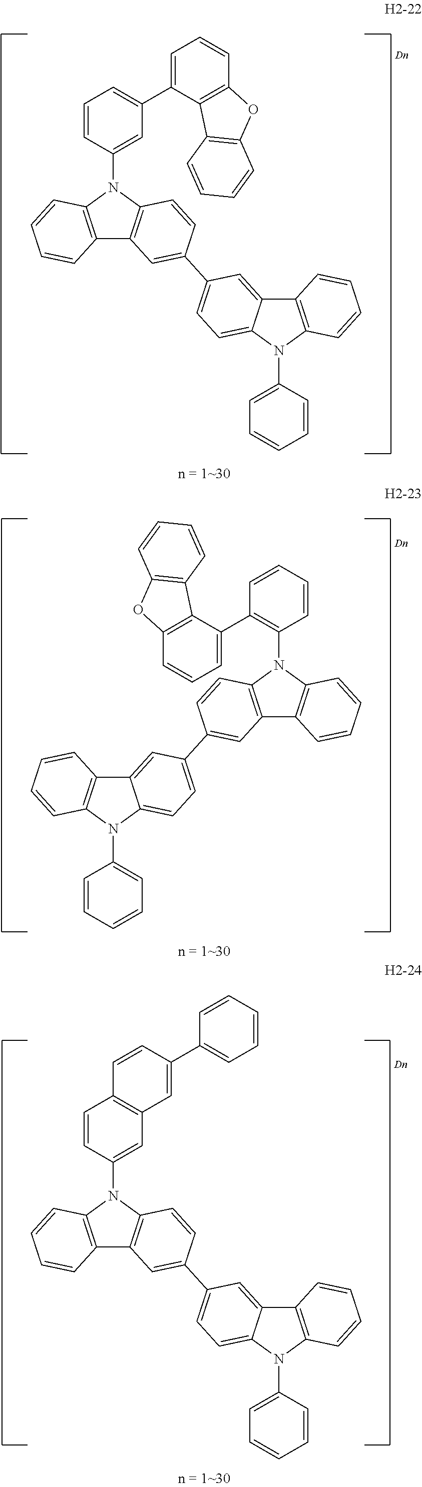

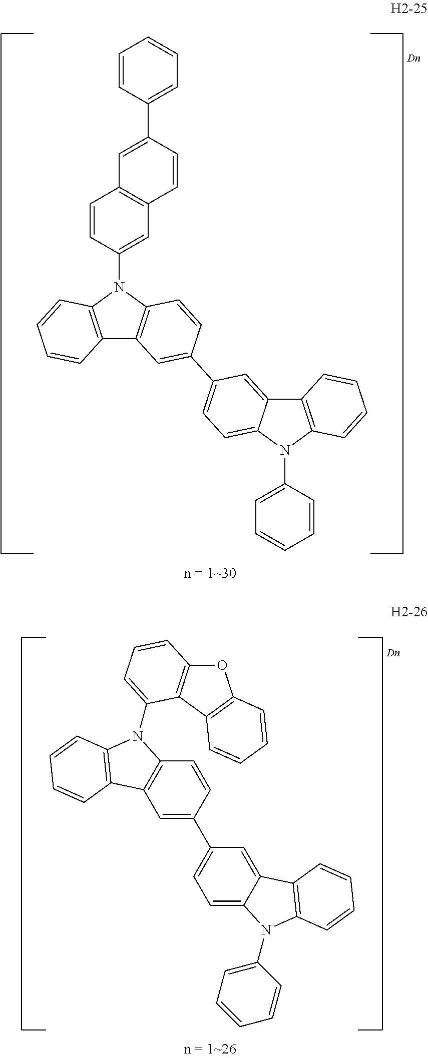

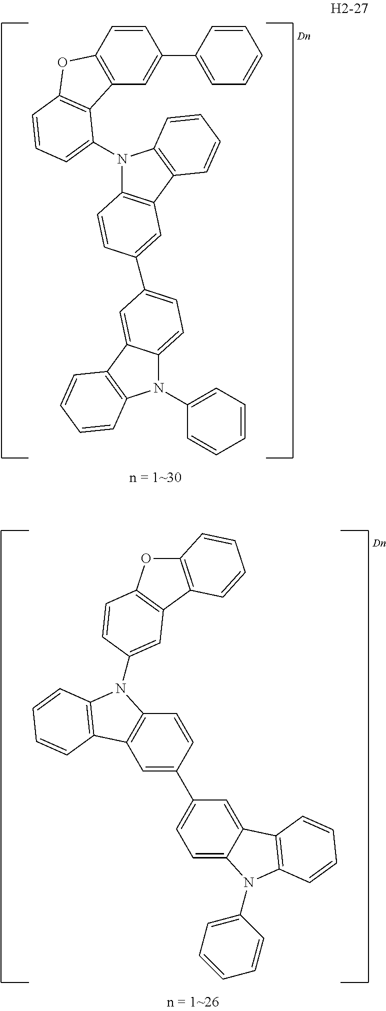

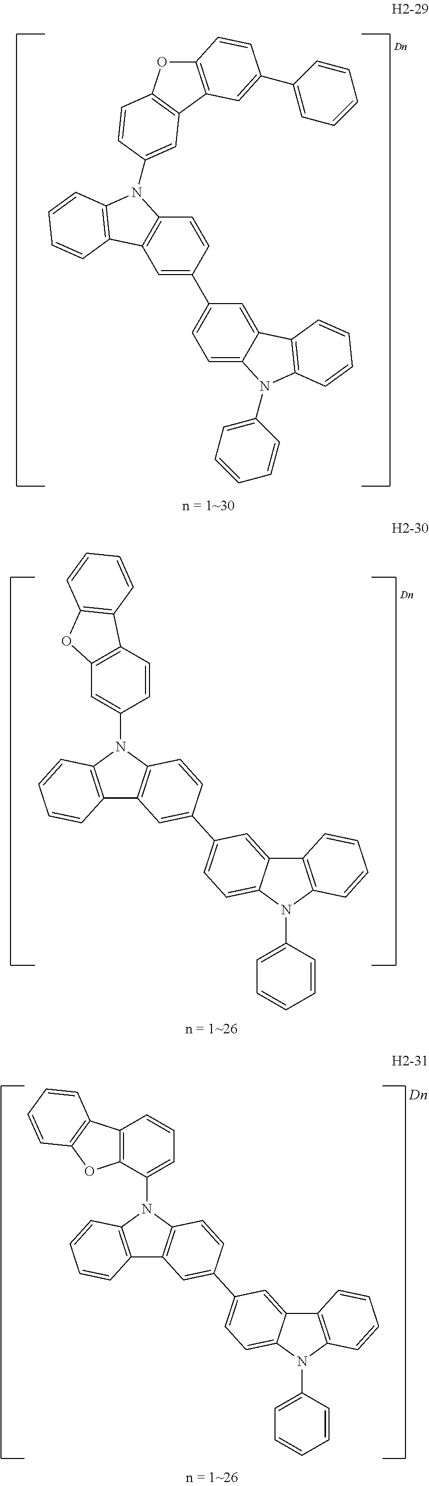

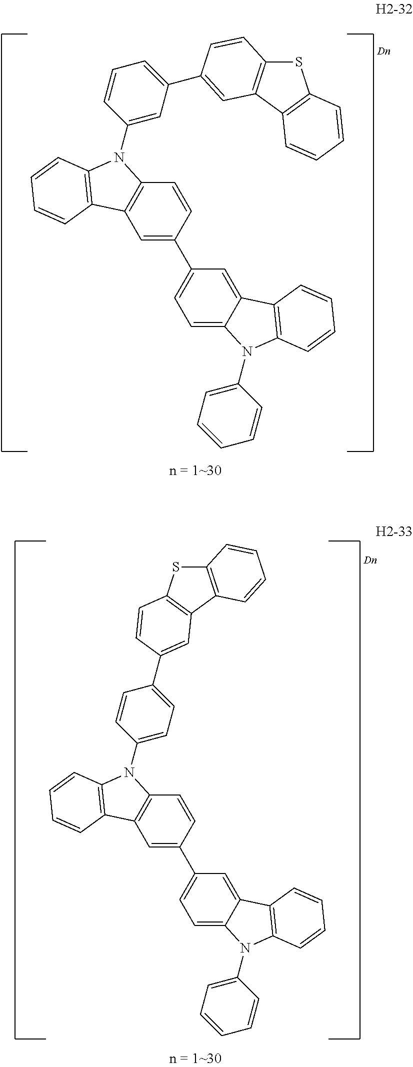

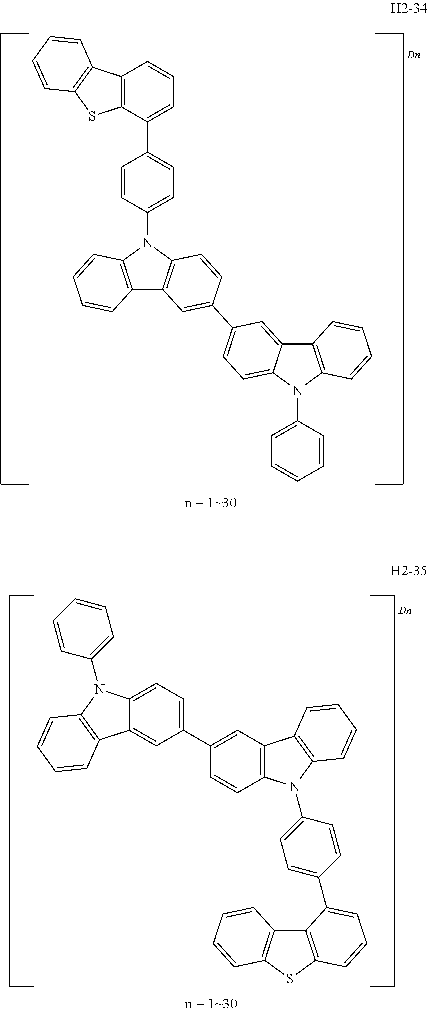

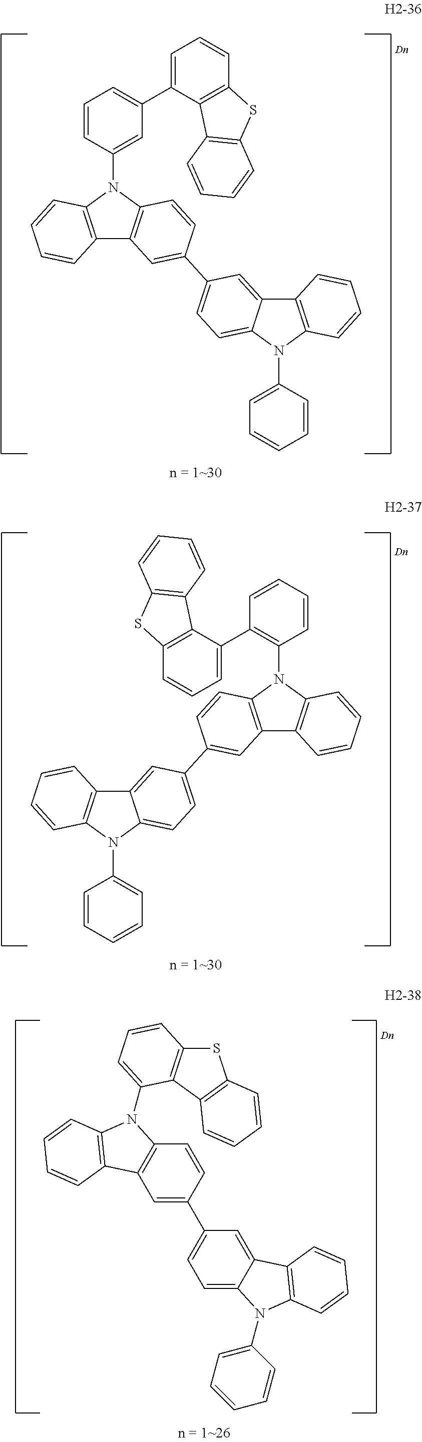

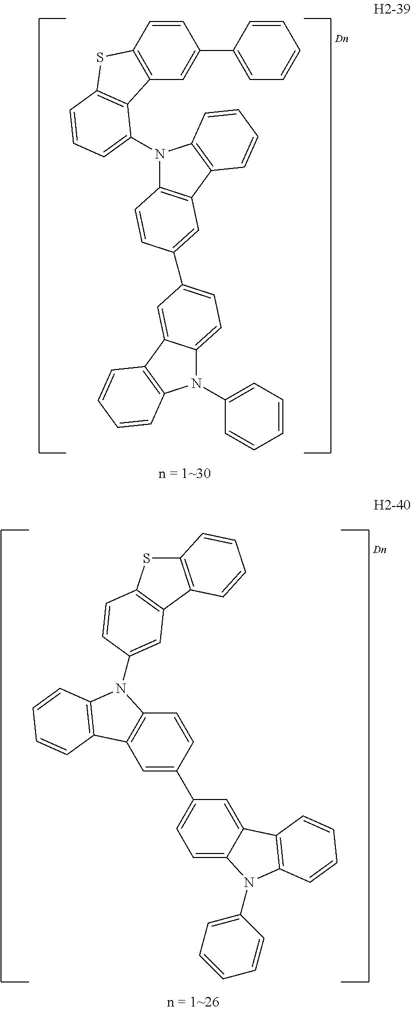

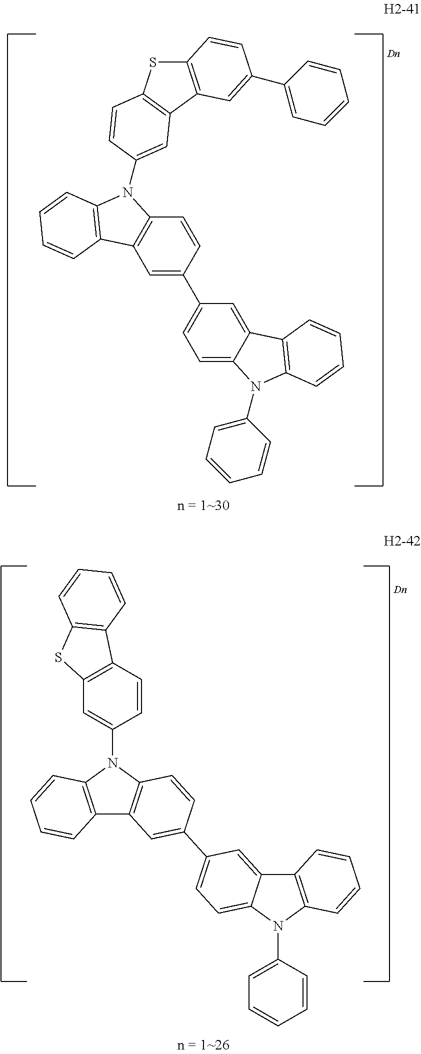

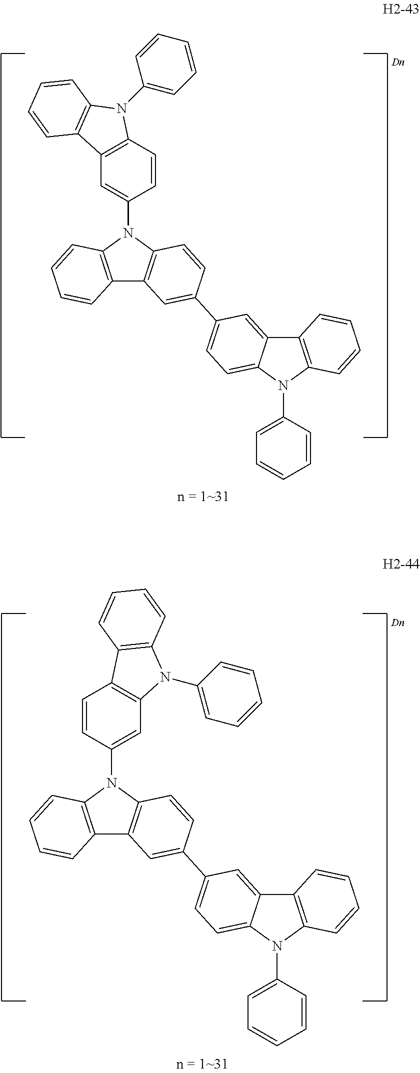

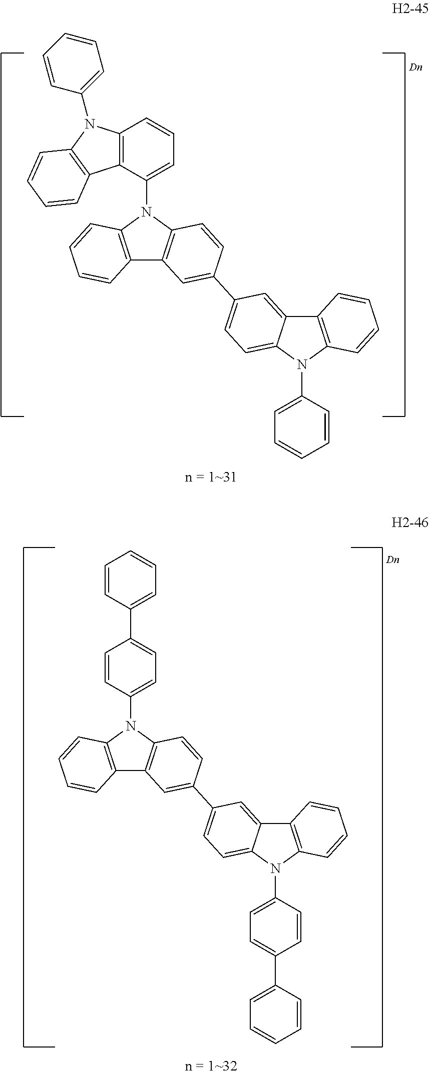

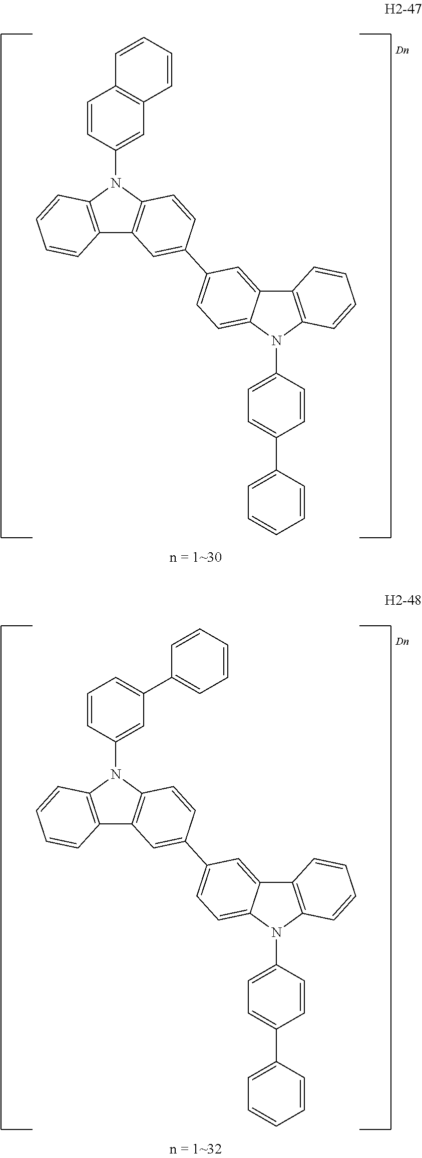

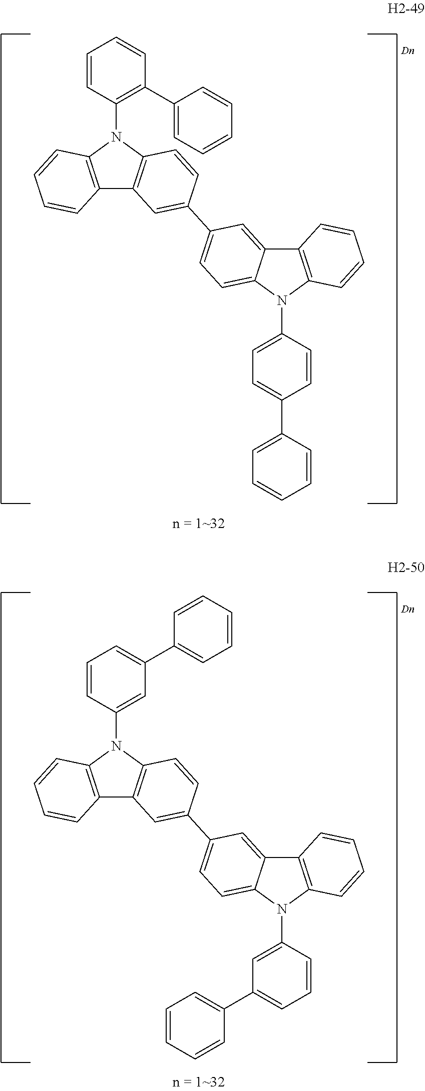

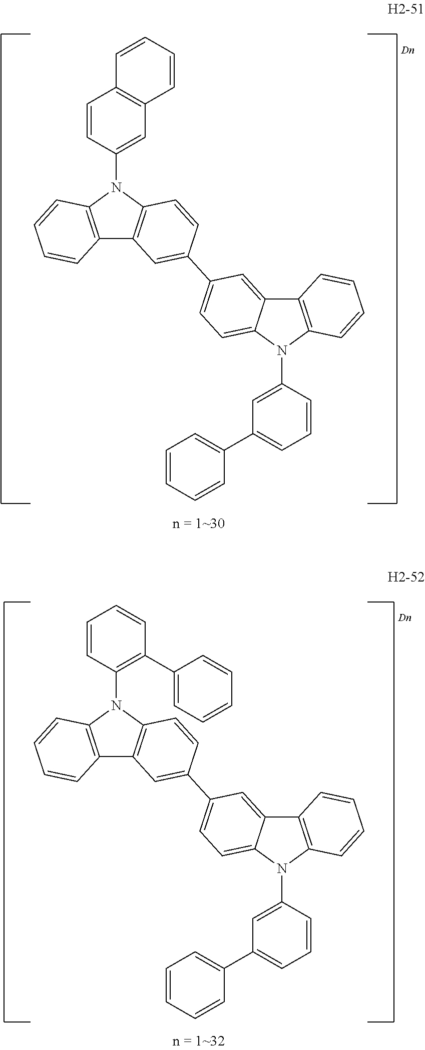

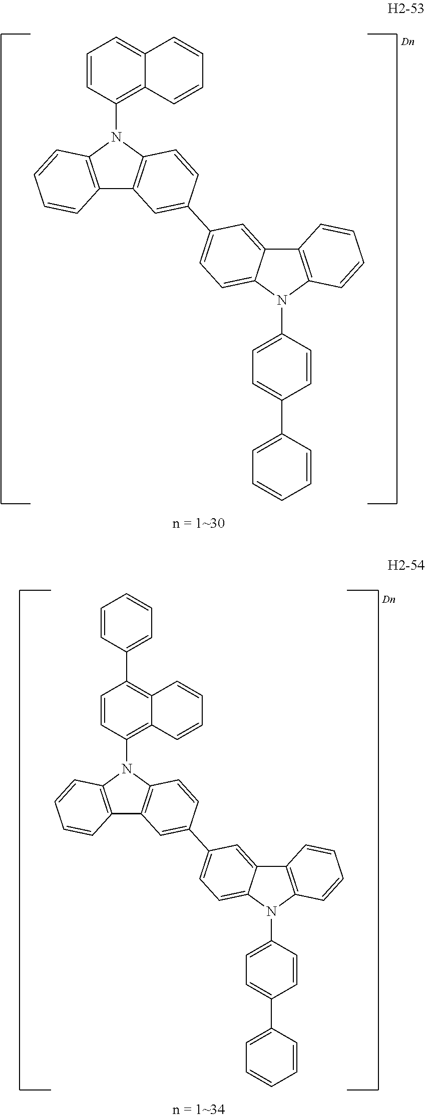

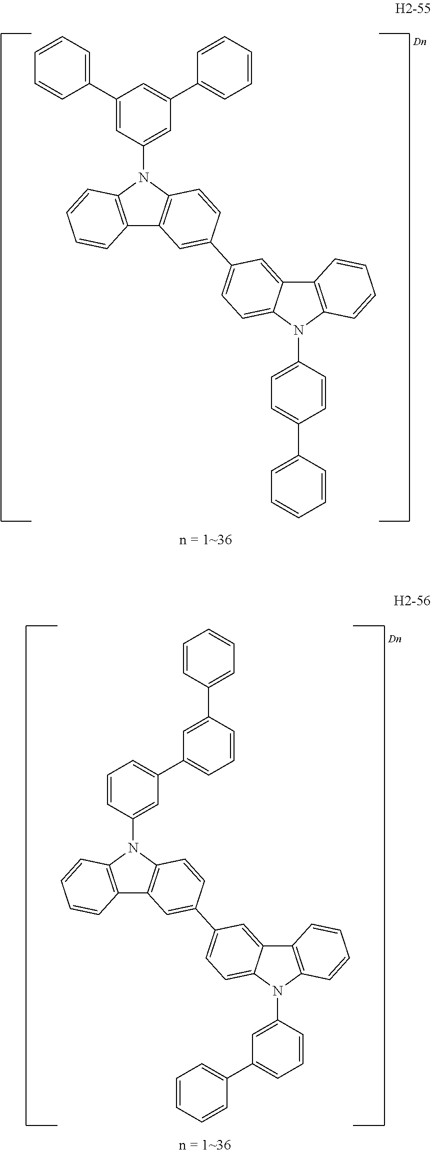

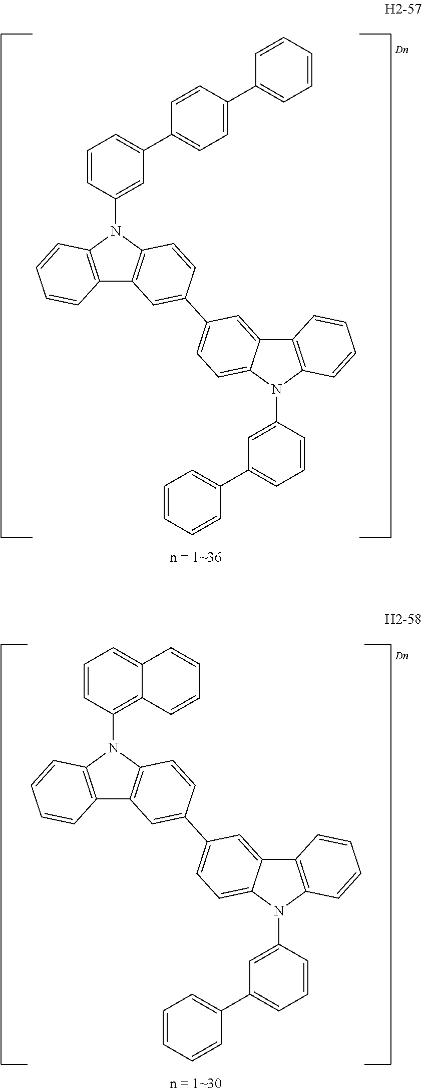

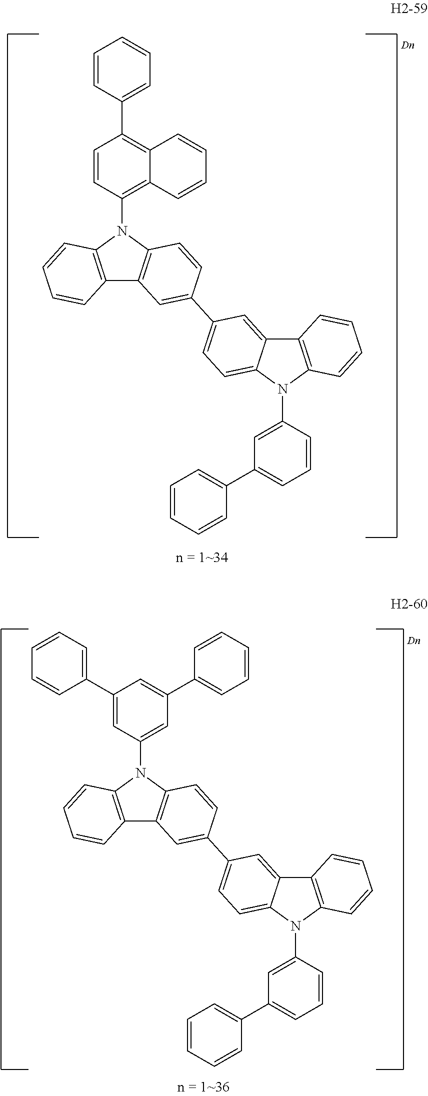

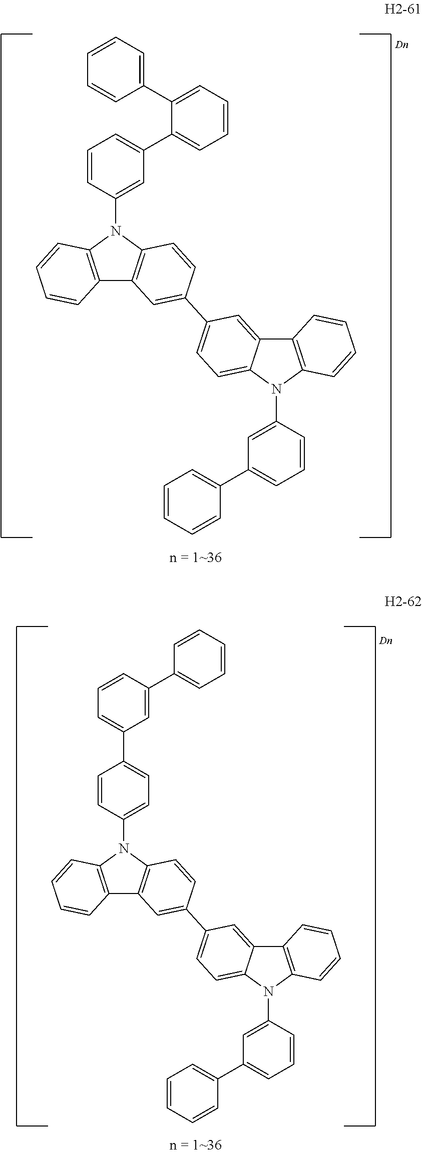

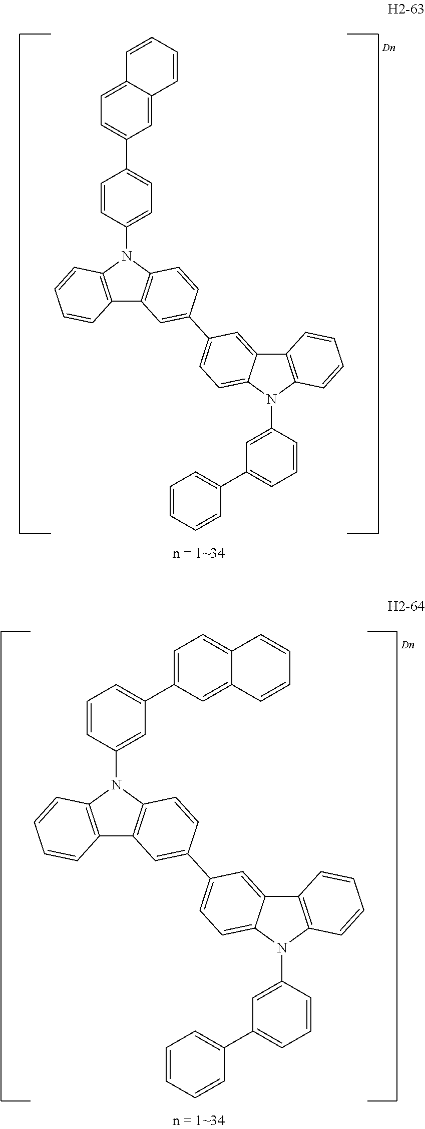

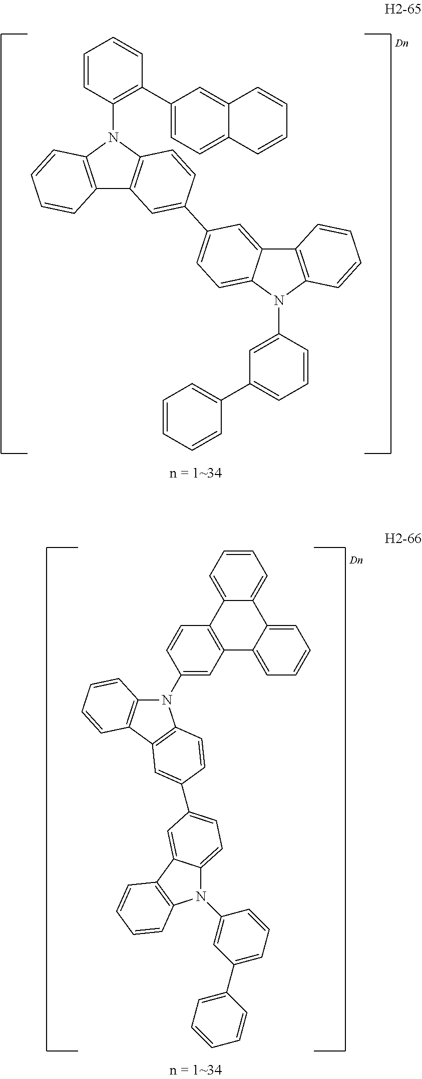

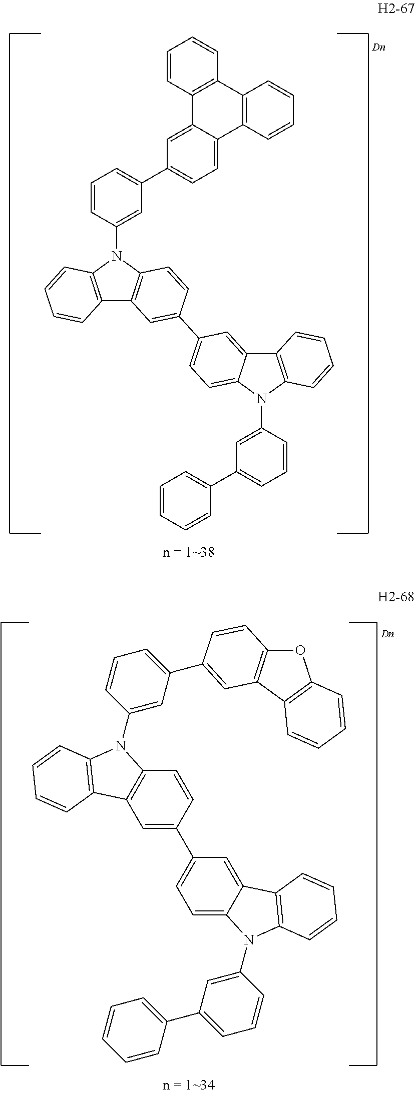

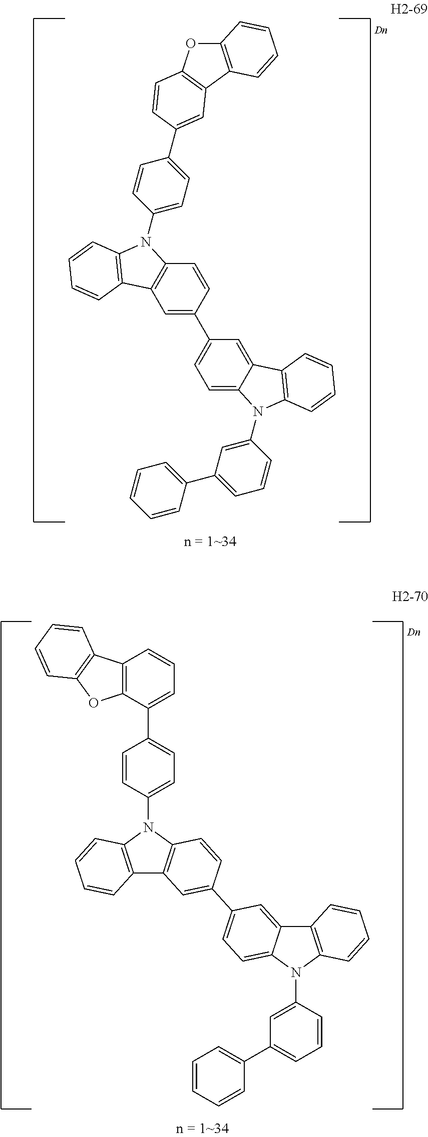

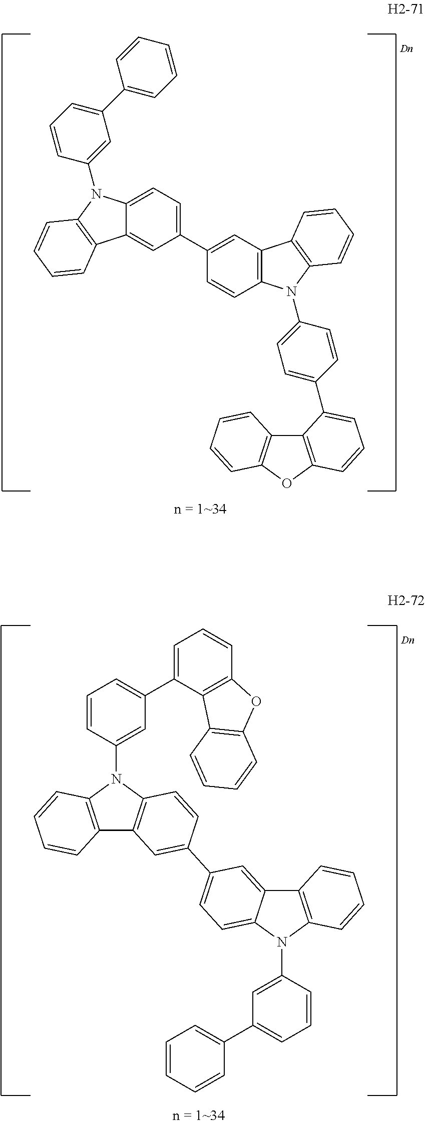

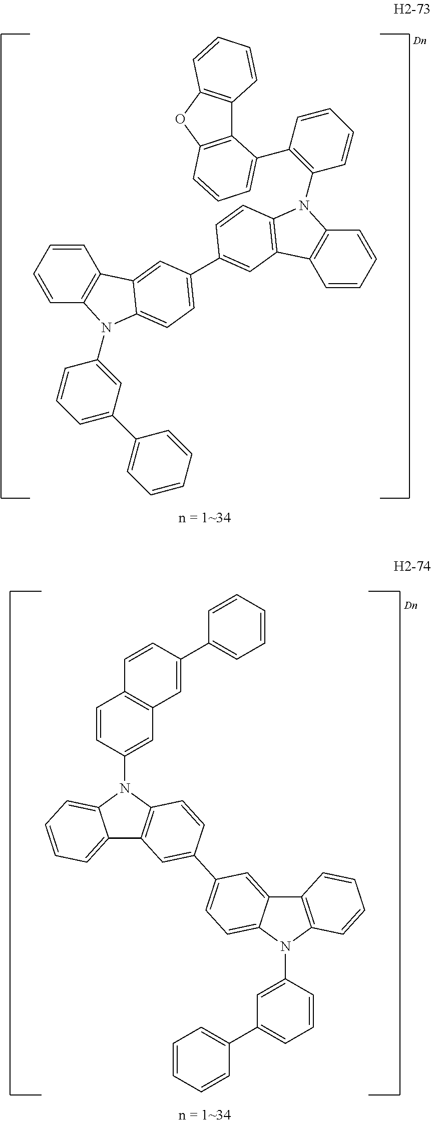

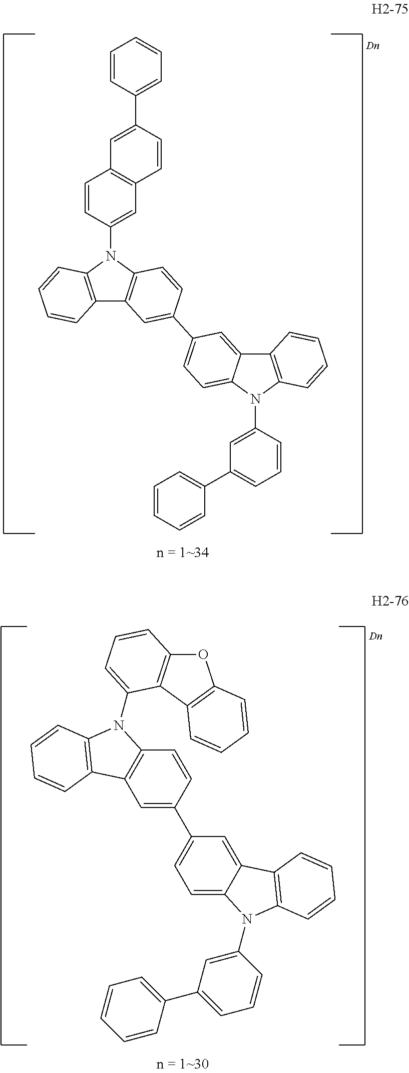

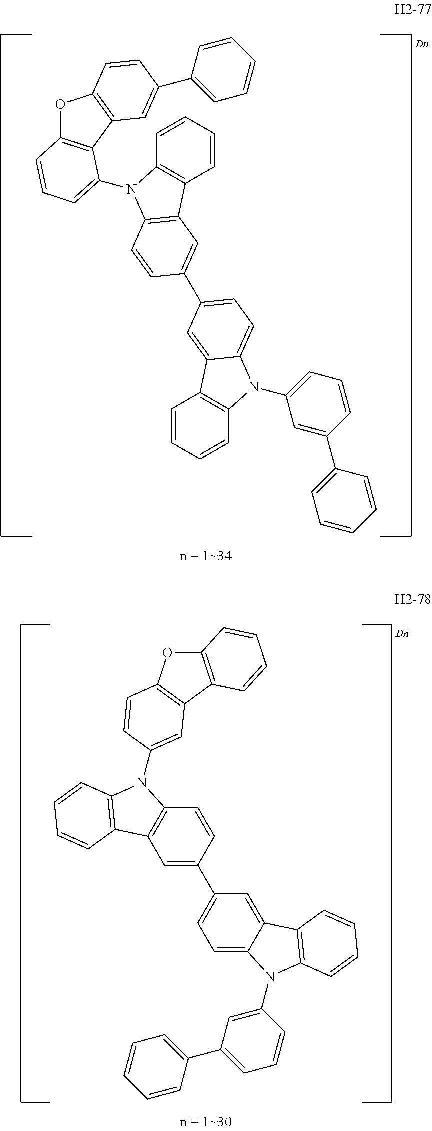

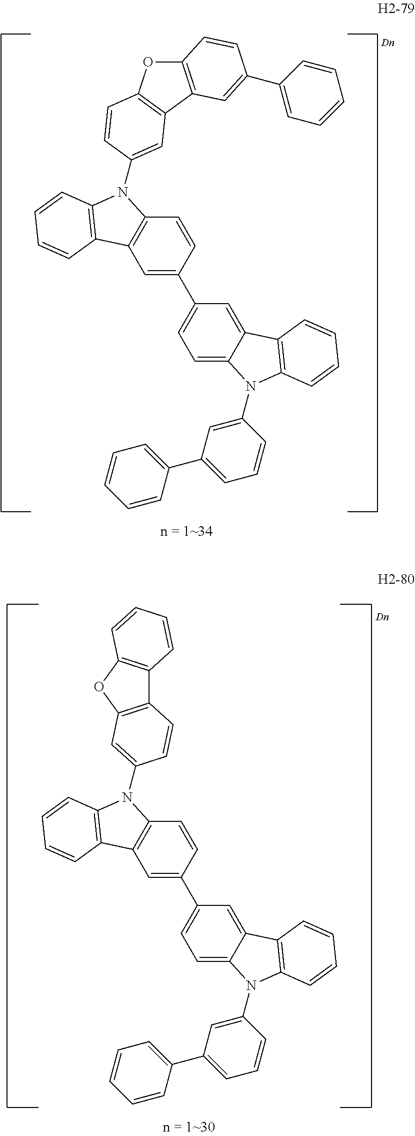

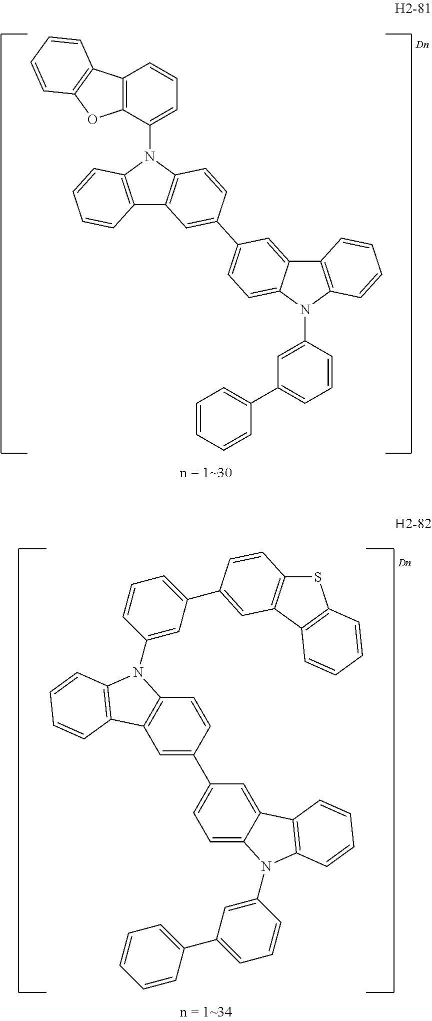

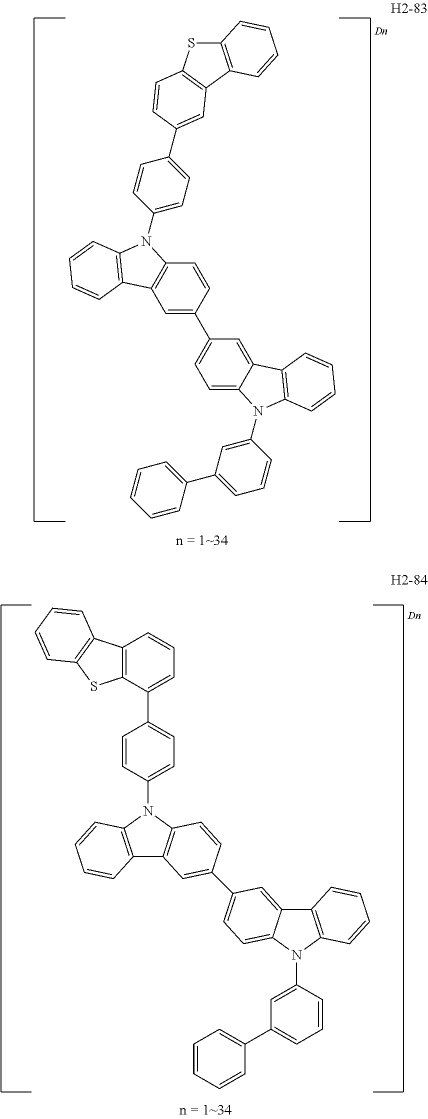

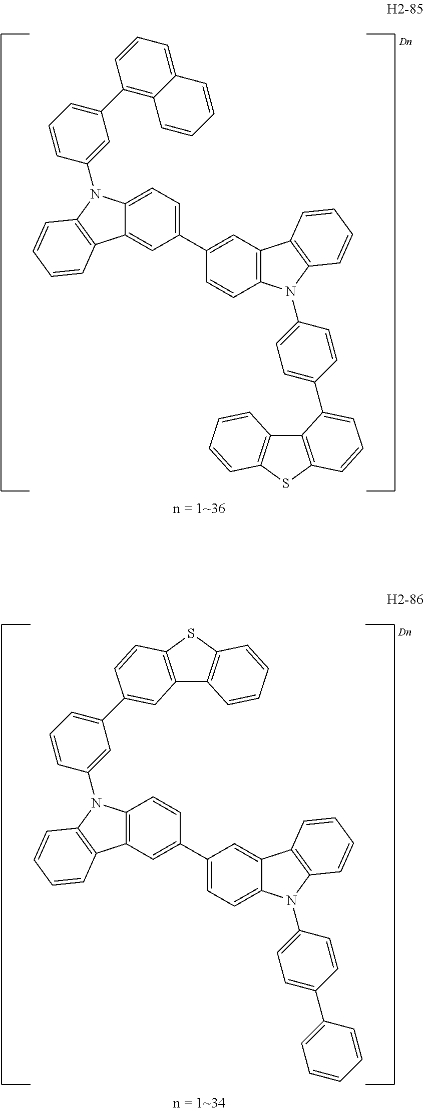

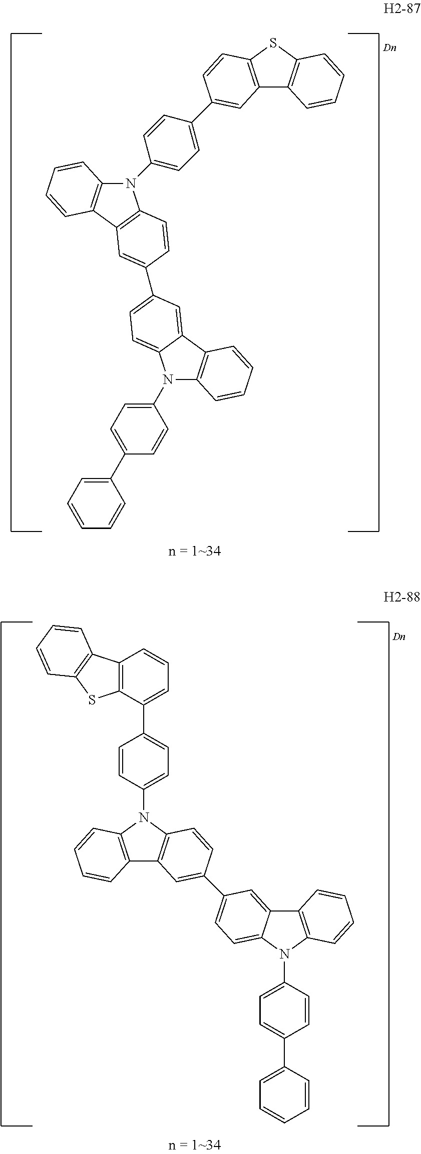

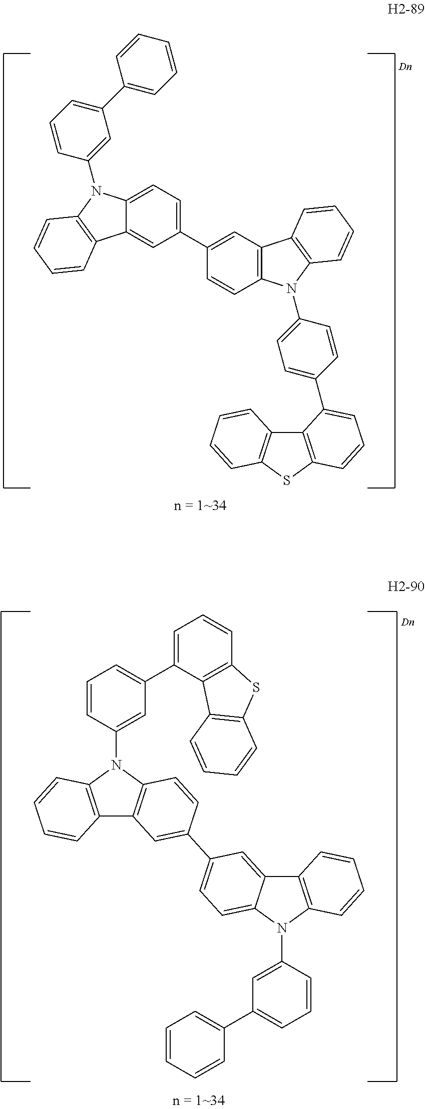

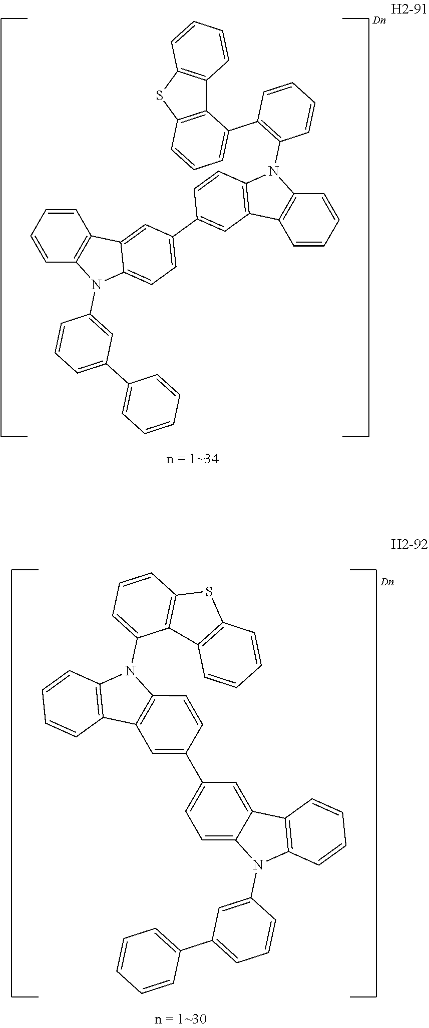

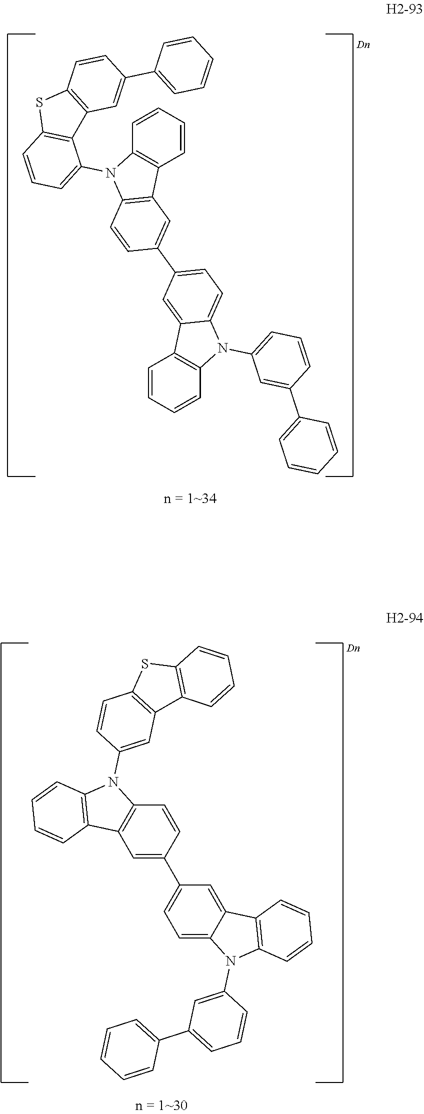

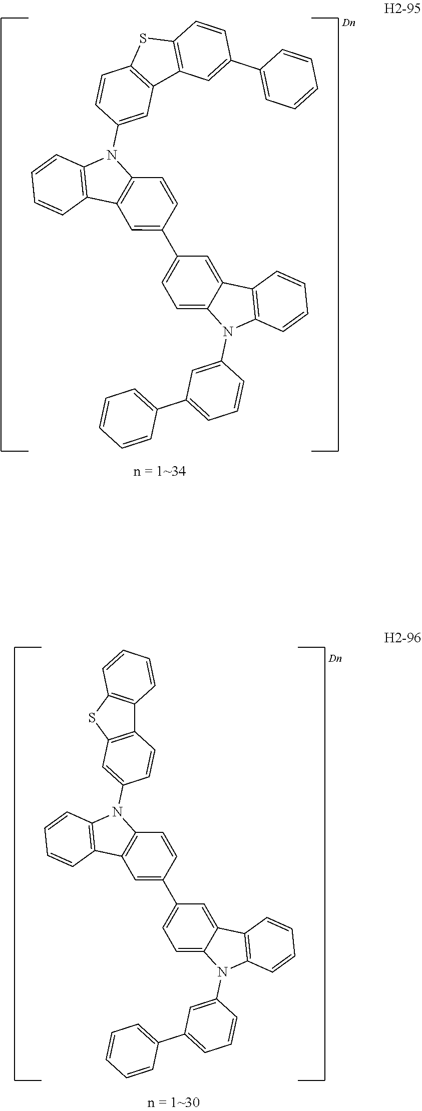

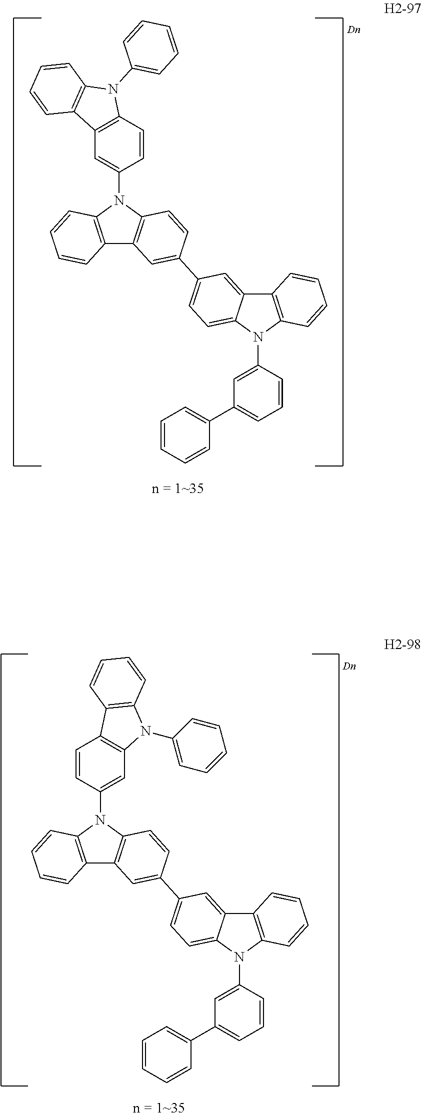

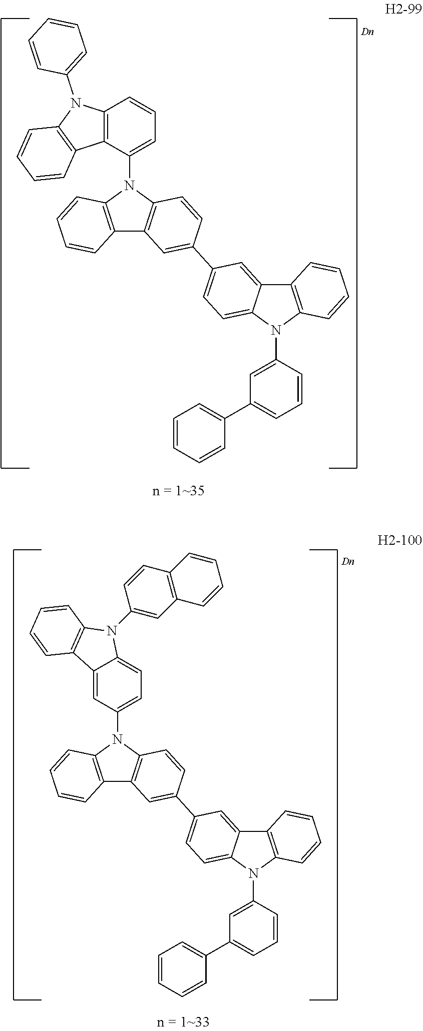

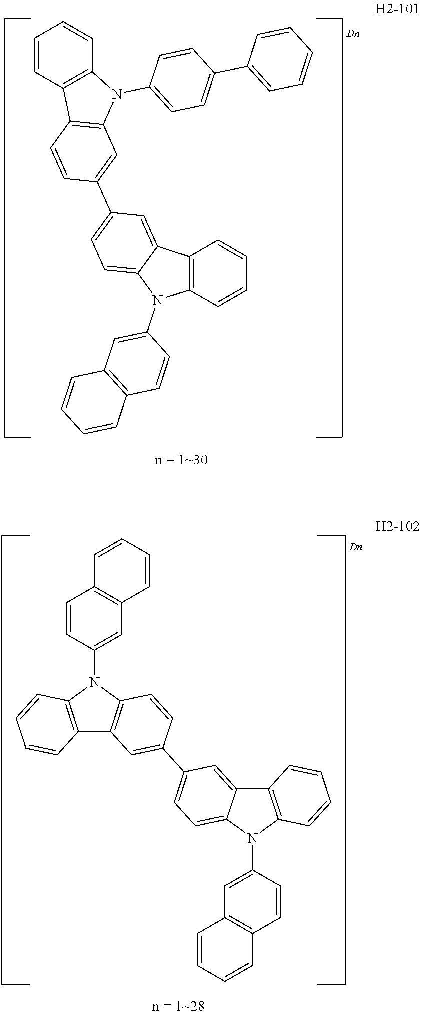

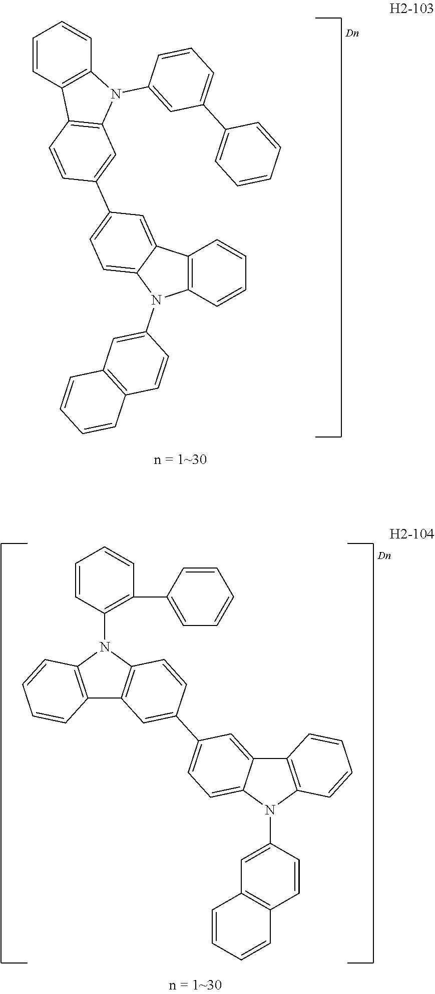

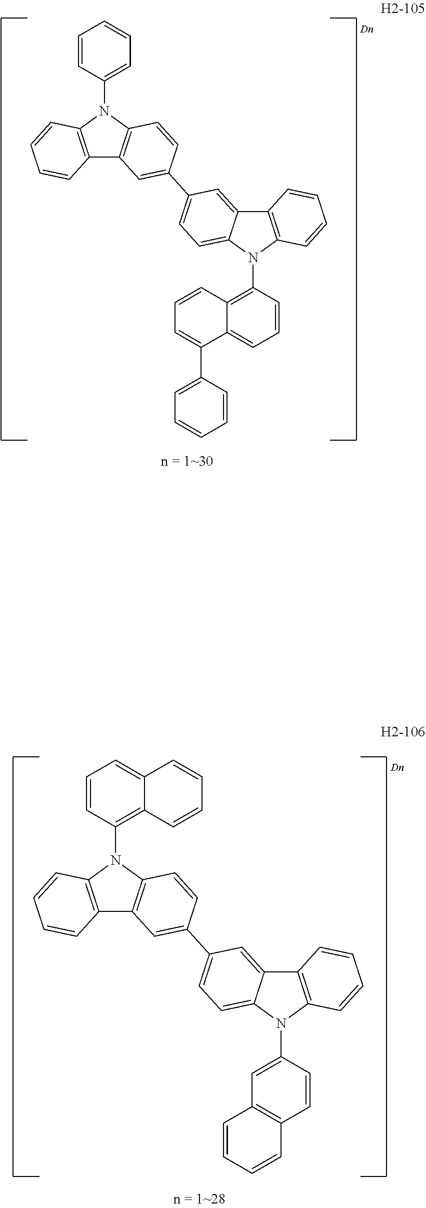

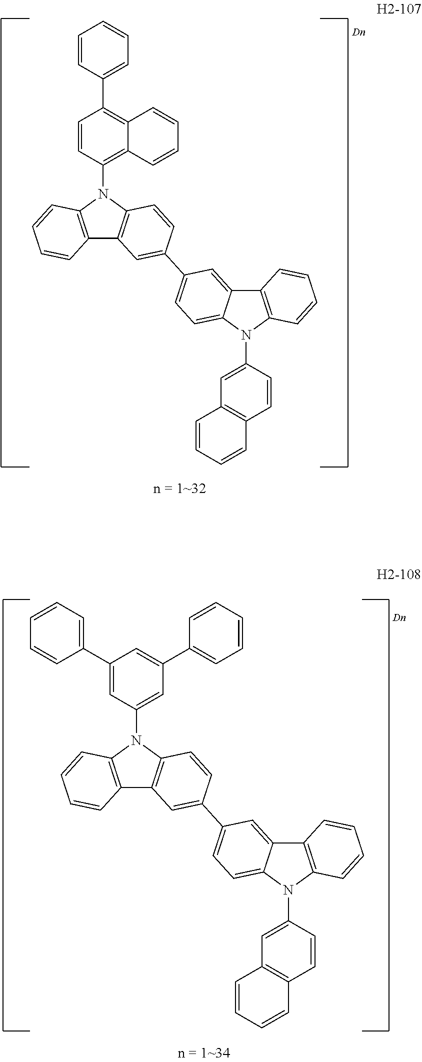

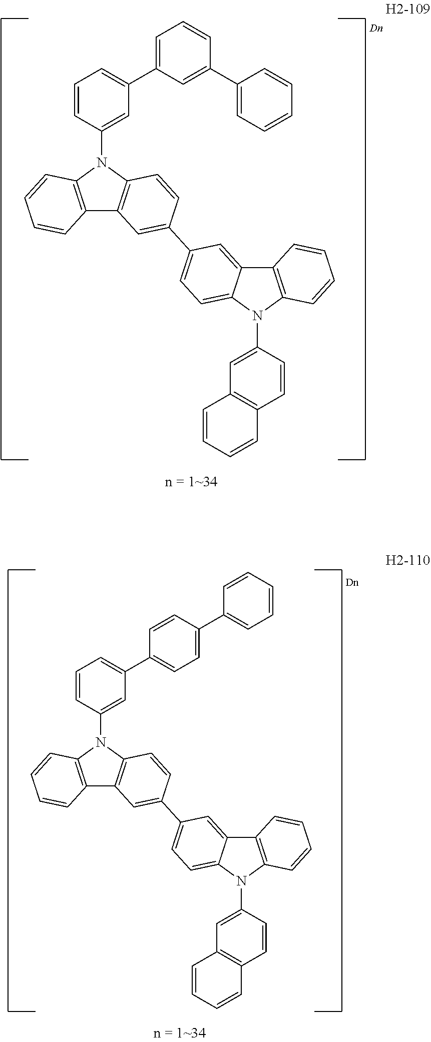

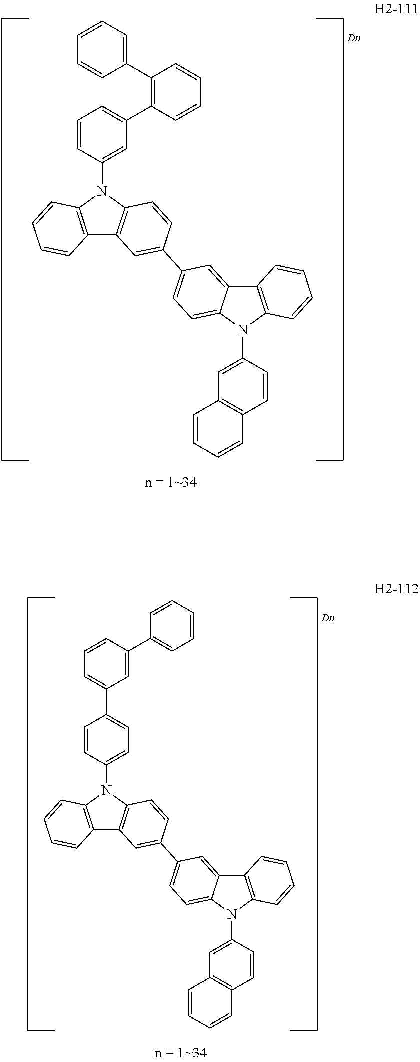

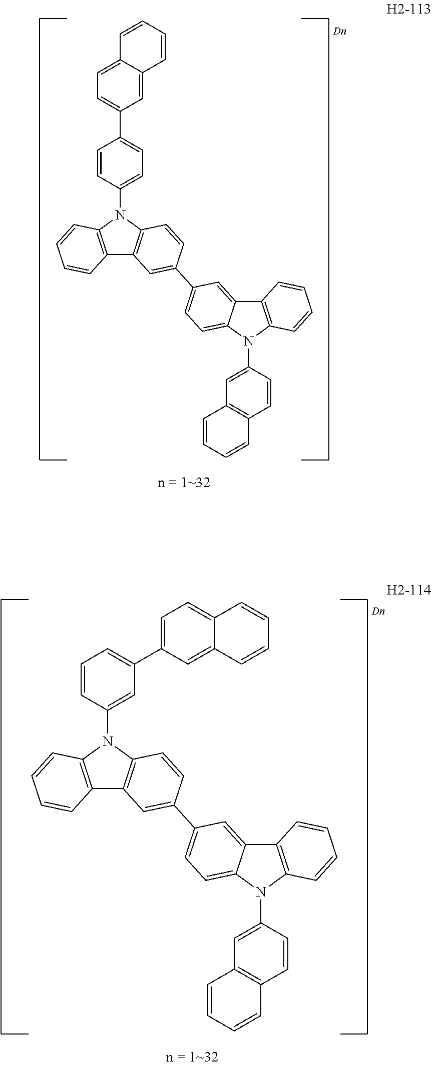

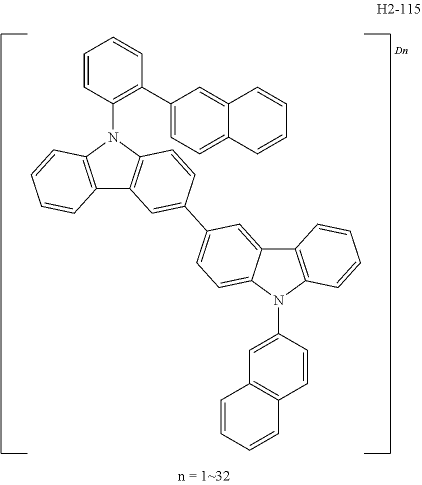

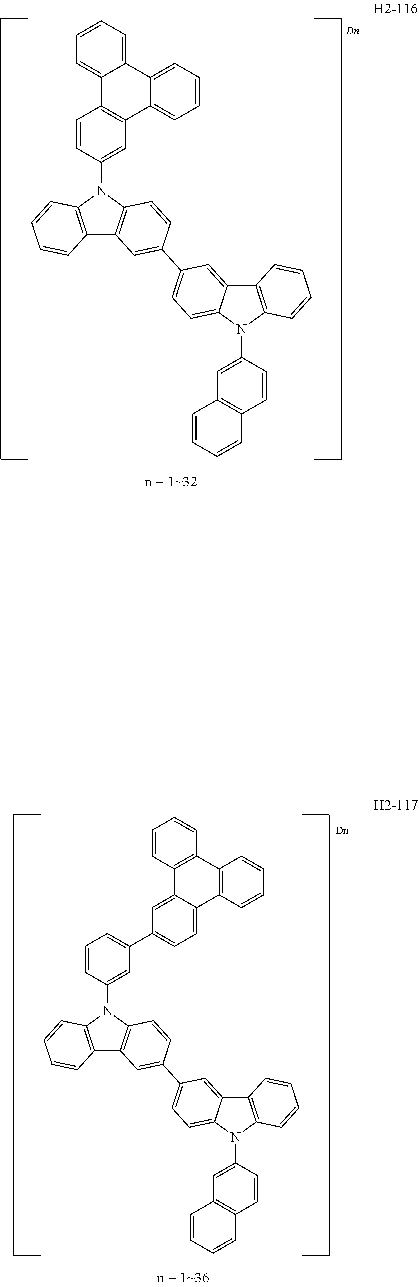

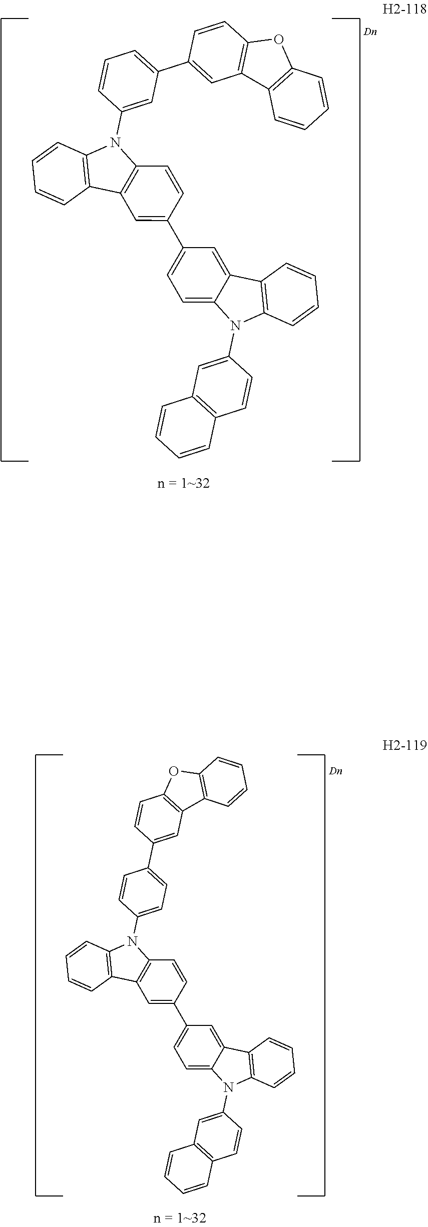

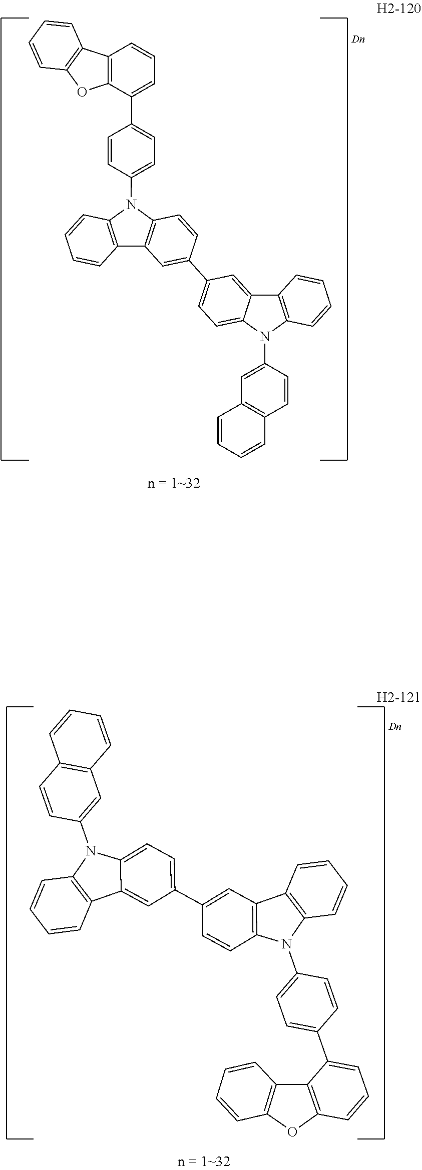

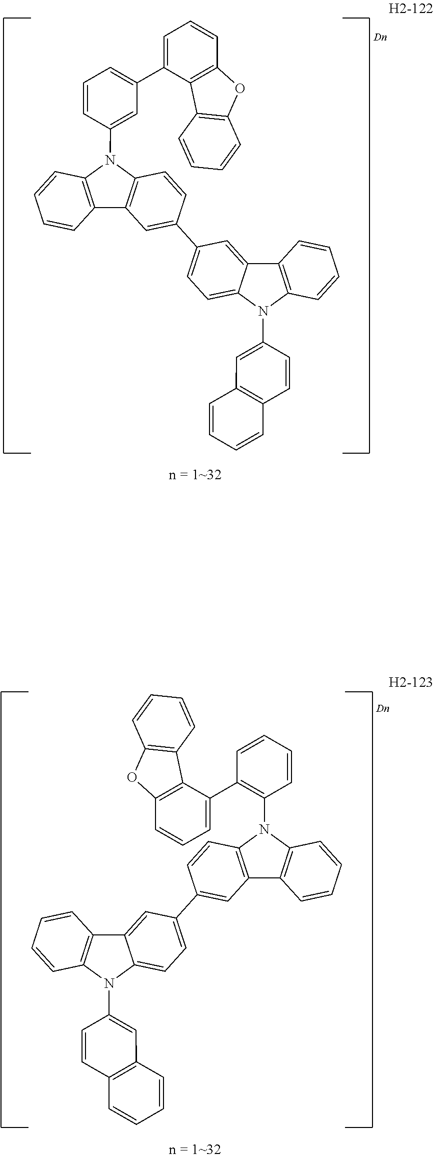

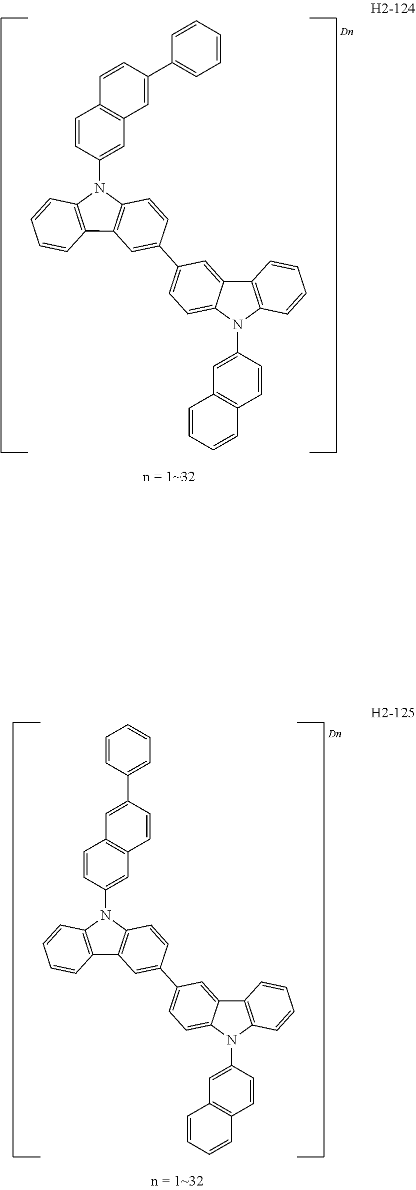

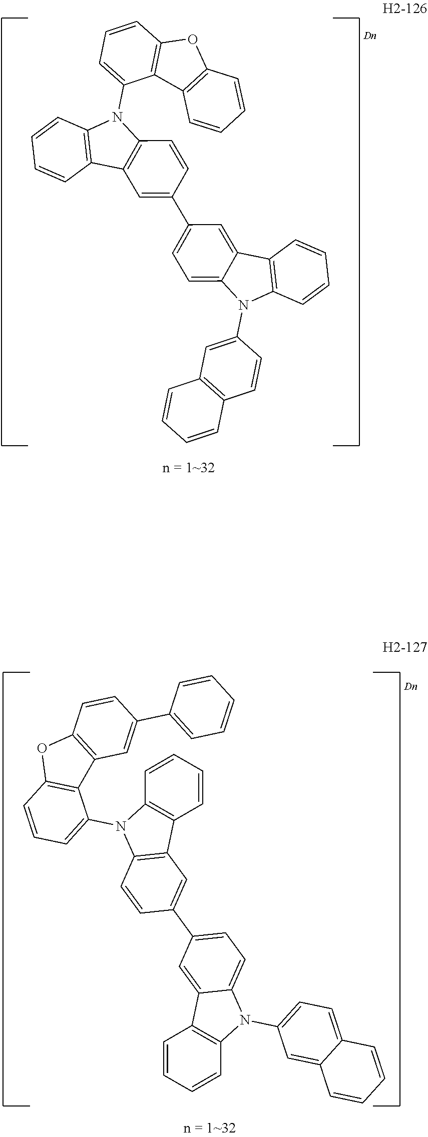

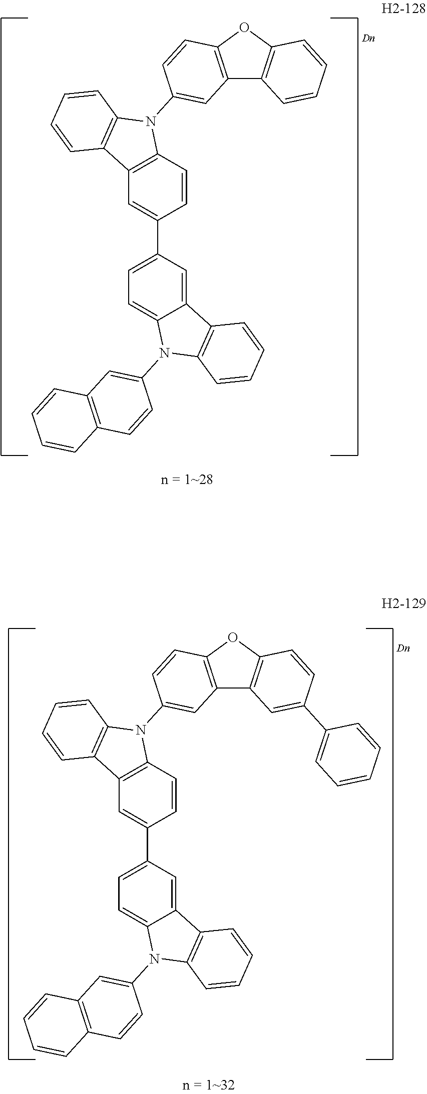

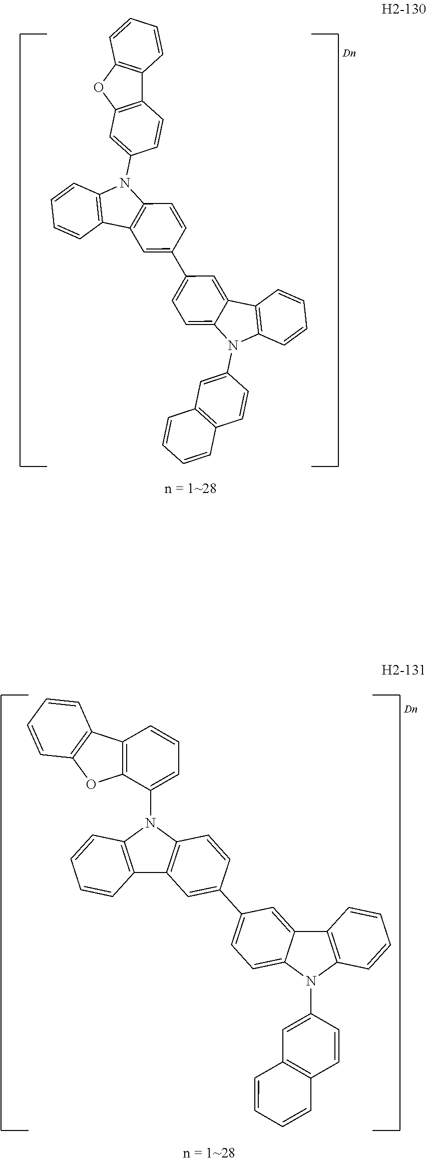

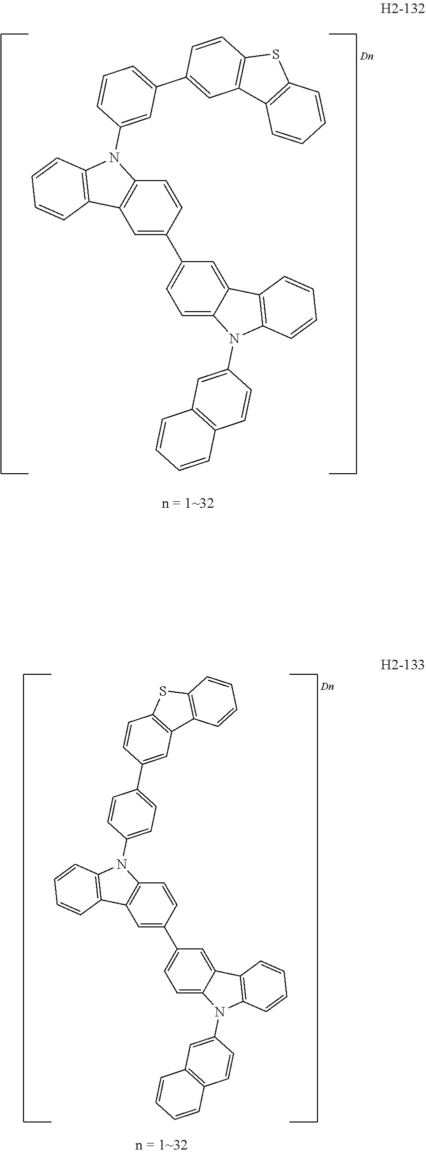

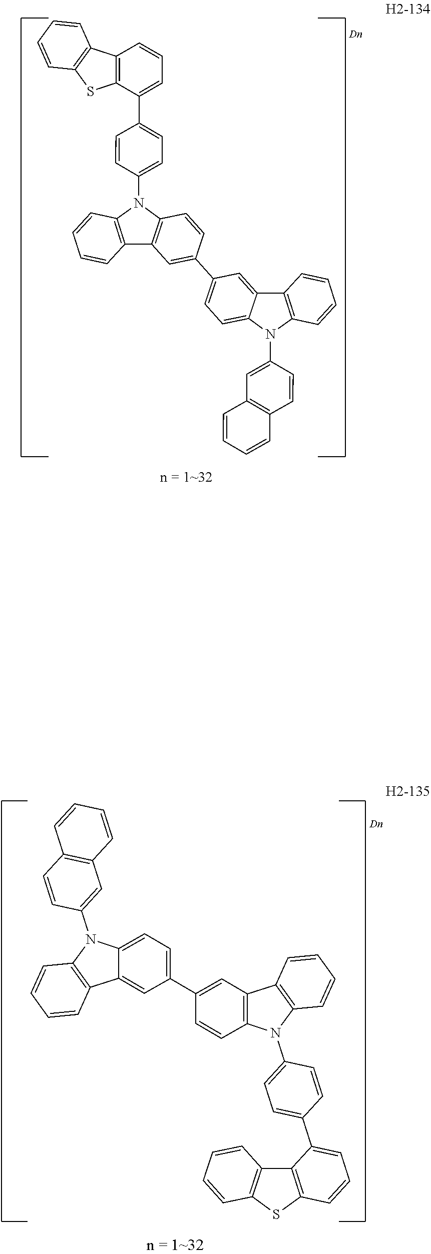

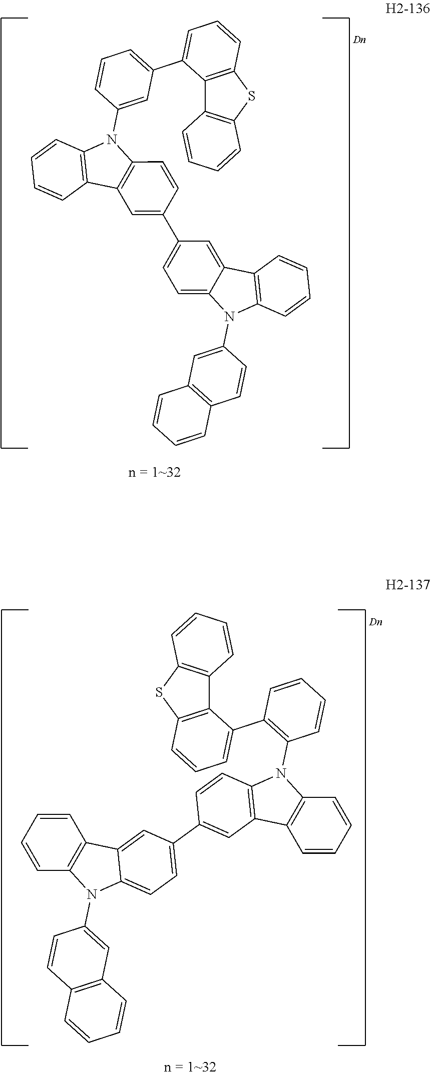

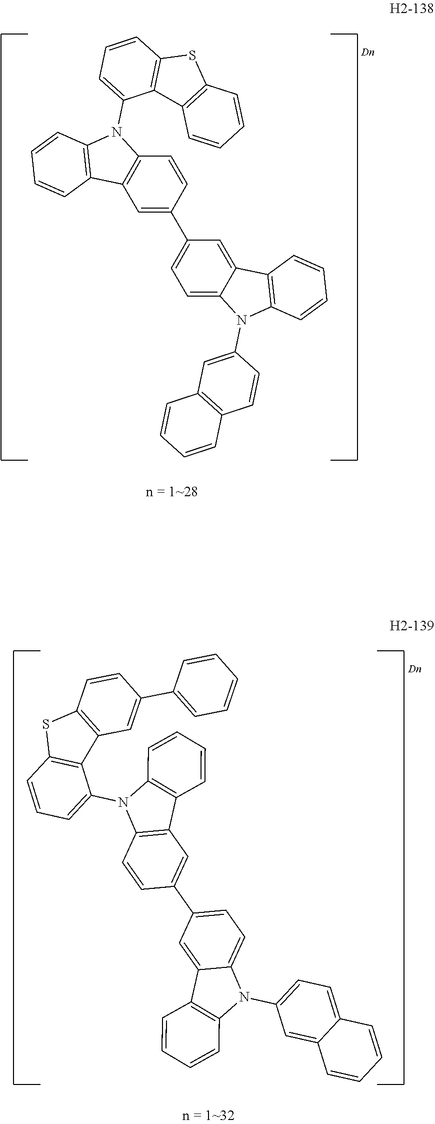

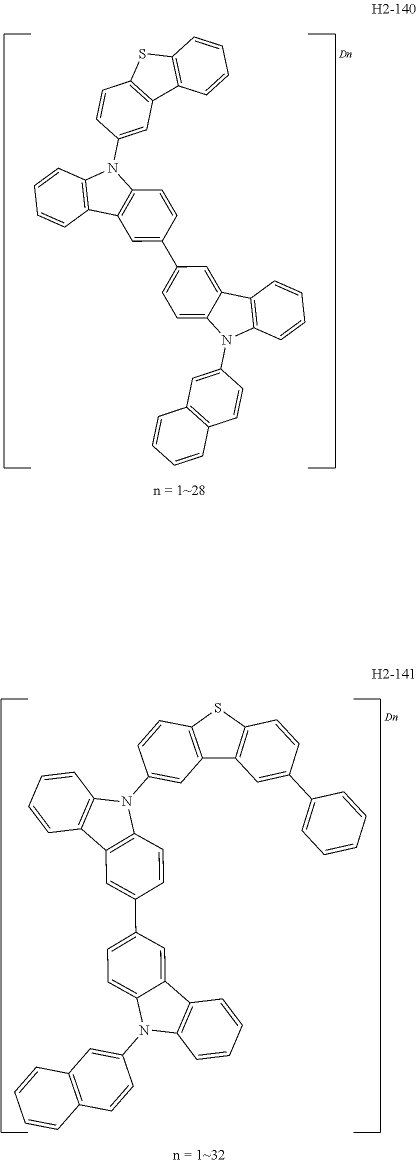

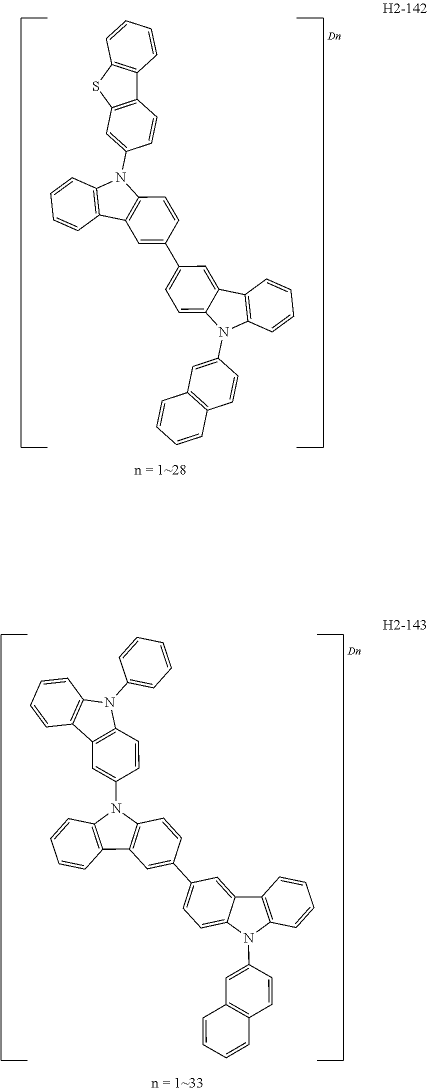

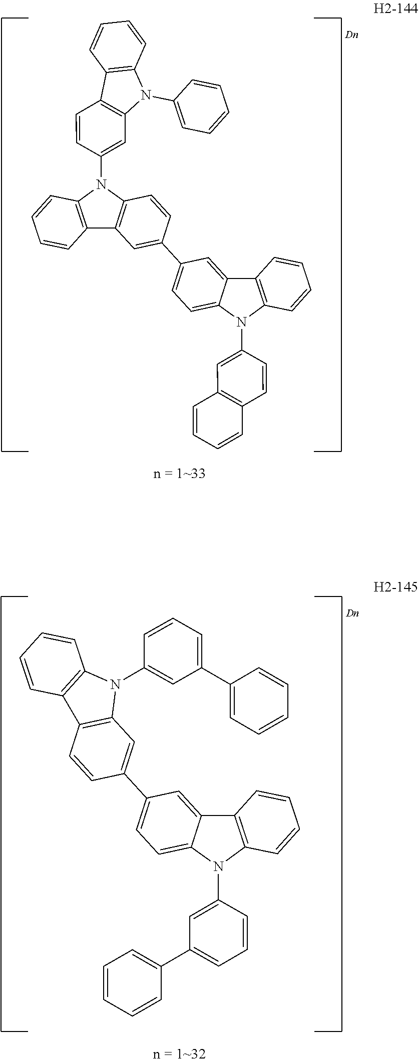

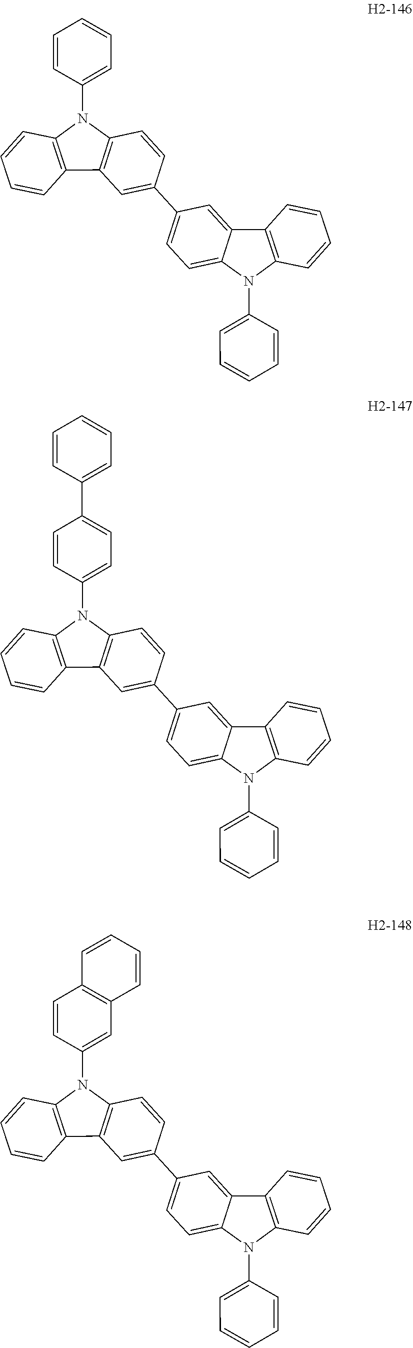 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1]

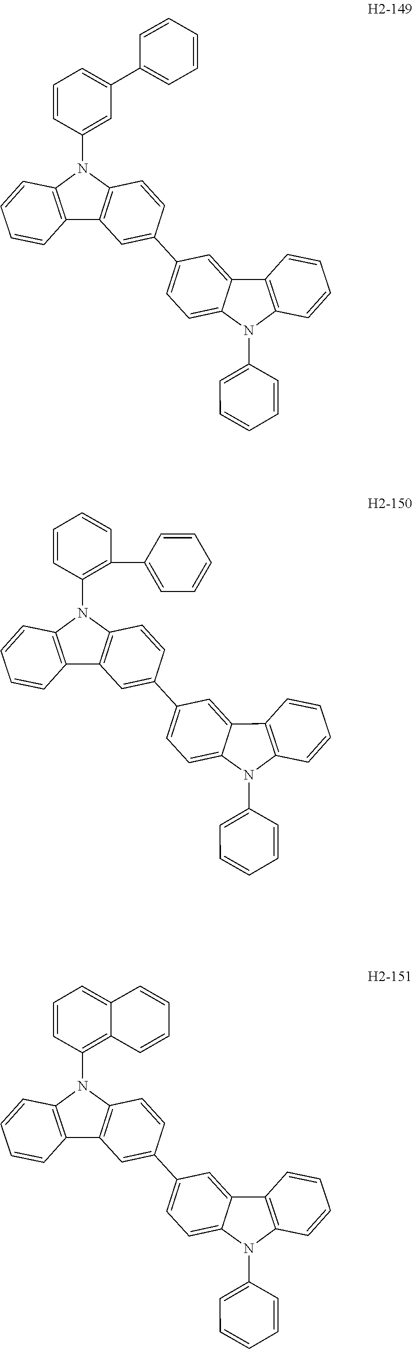 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1]

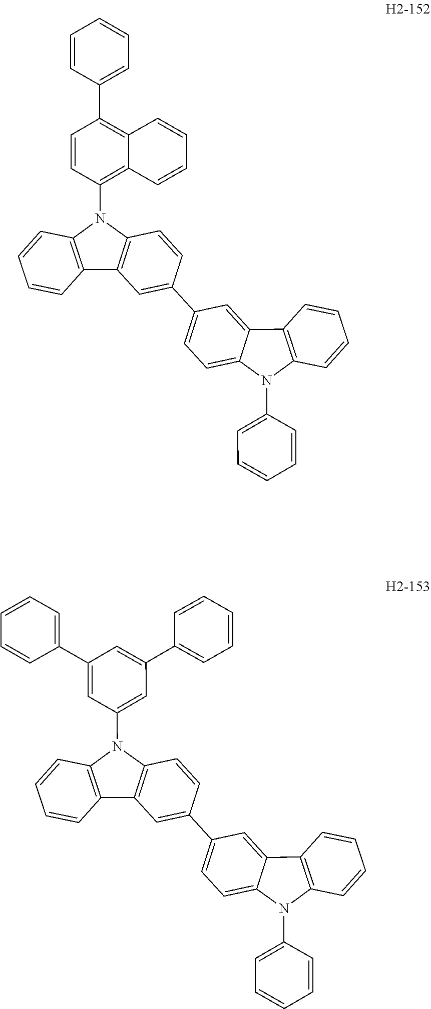 [SMILES: c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1]

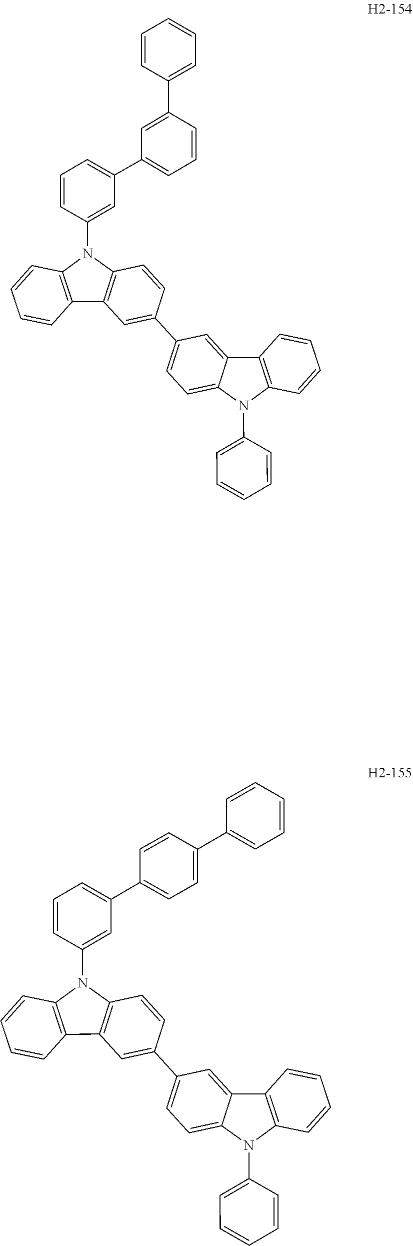 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

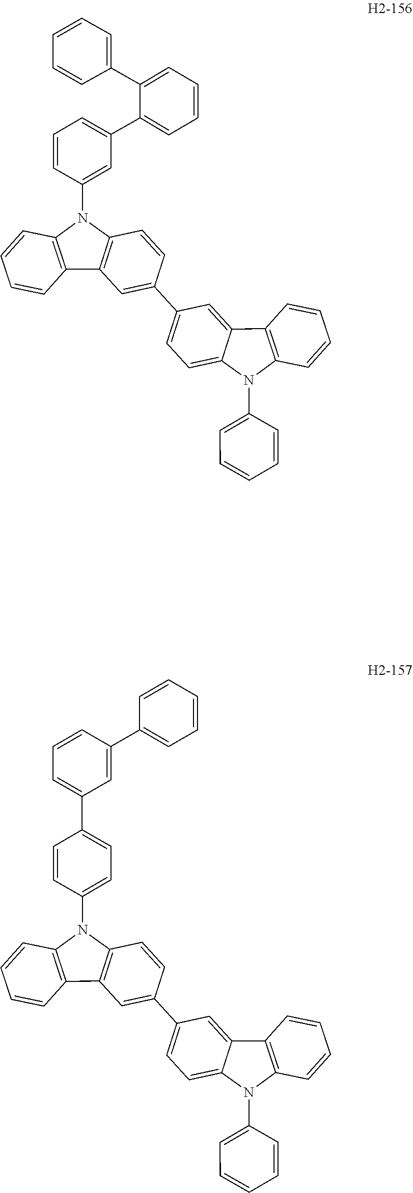 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

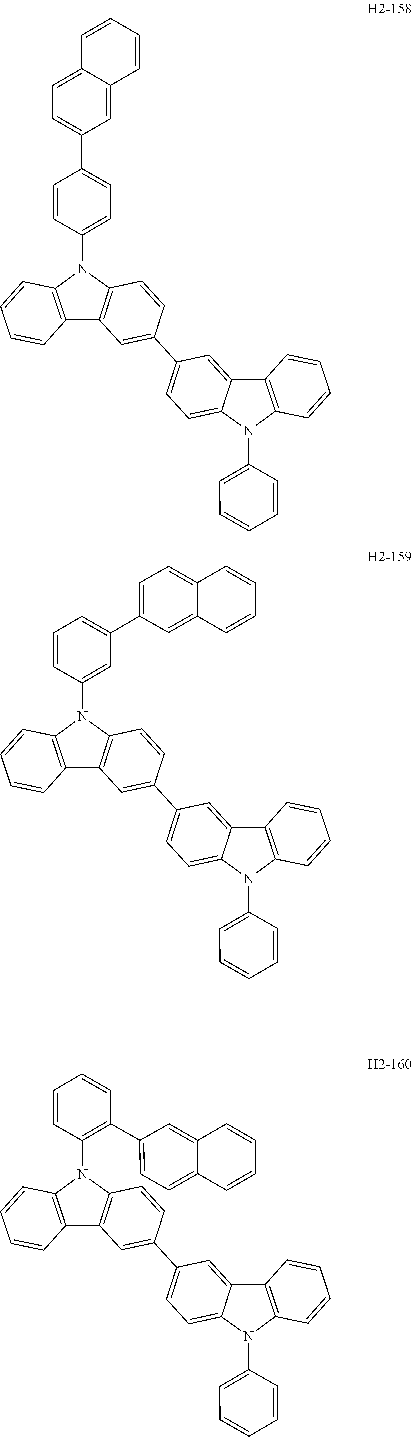

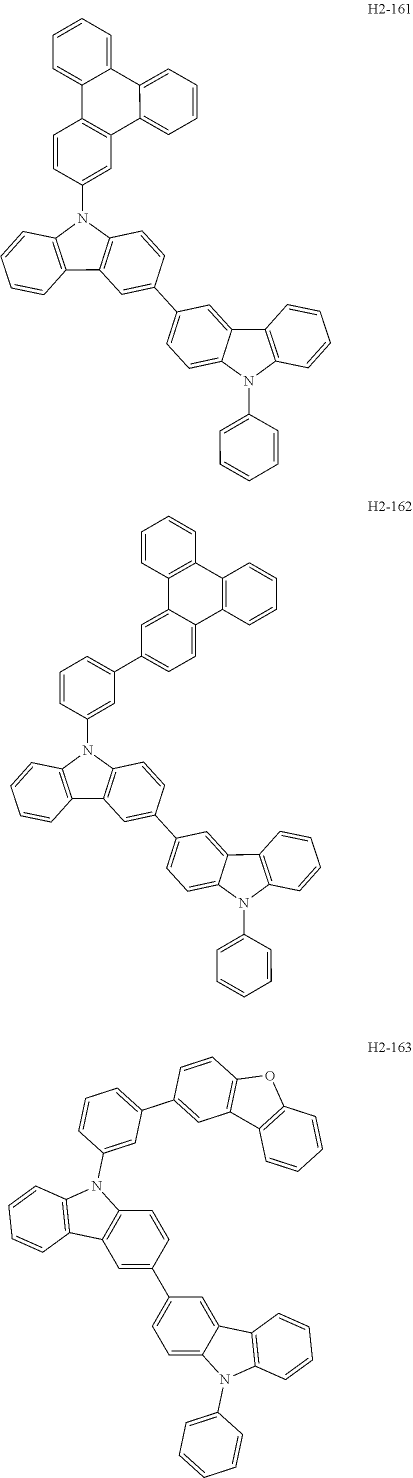 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3cc(-c4ccc5c(c4)c4ccccc4n5-c4cccc(-c5ccc6oc7ccccc7c6c5)c4)ccc32)cc1]

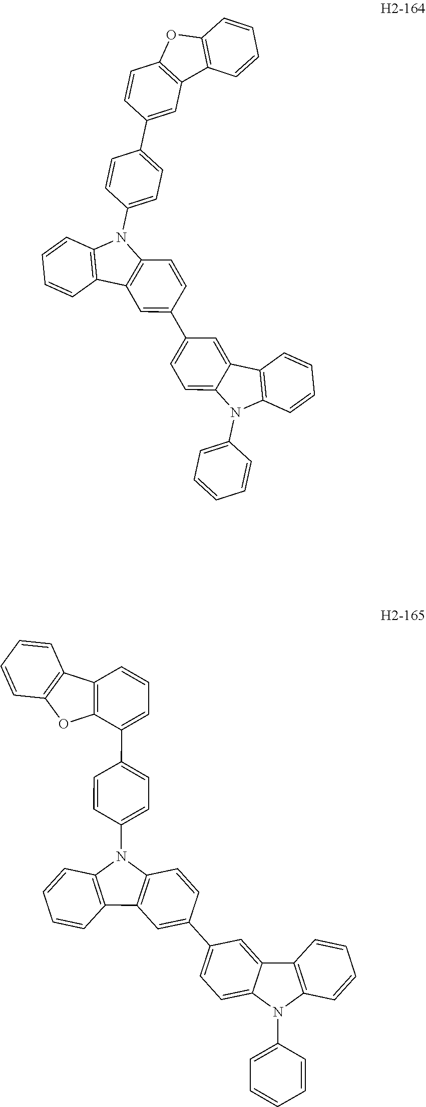 [SMILES: c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6c5oc5ccccc56)cc4)ccc32)cc1]

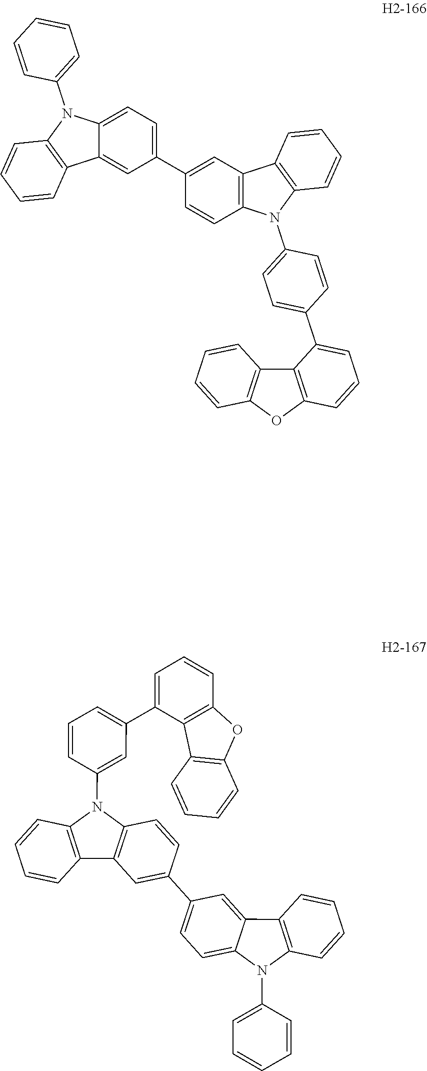 [SMILES: c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1]

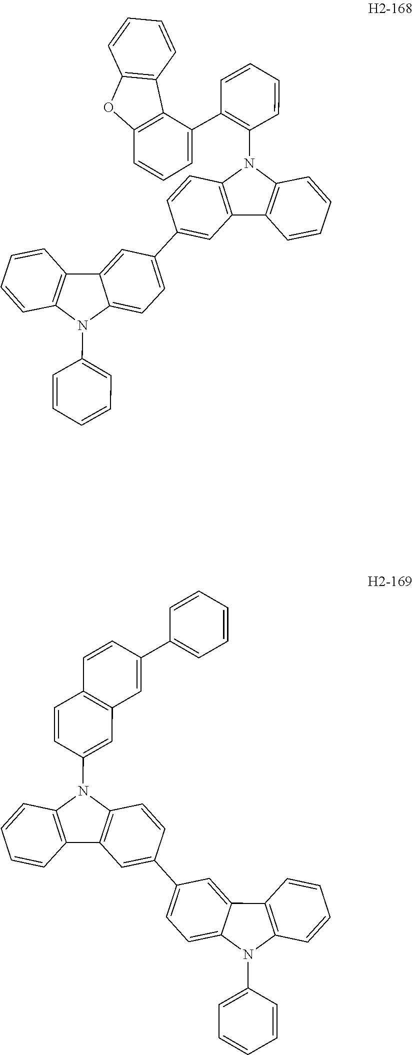 [SMILES: c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1]

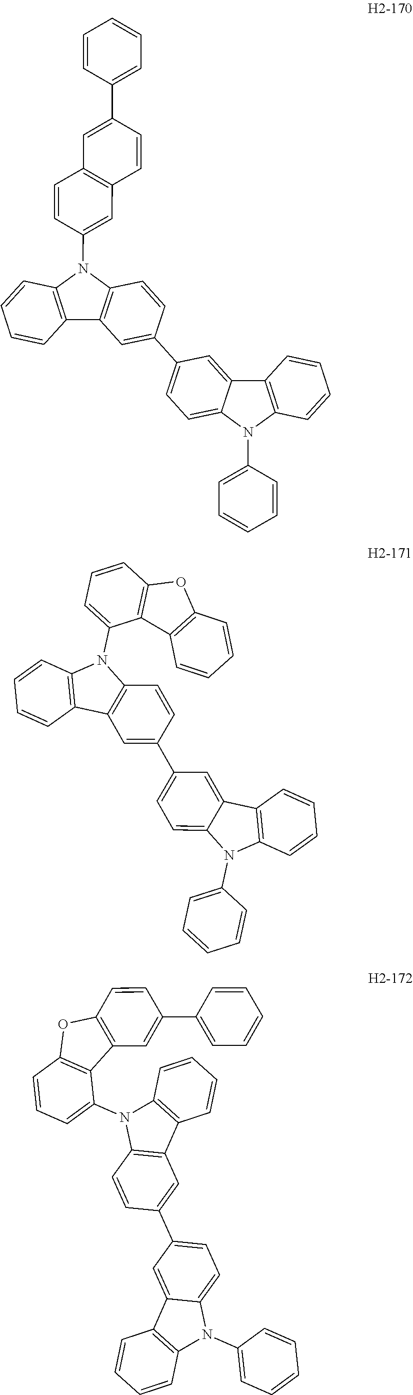

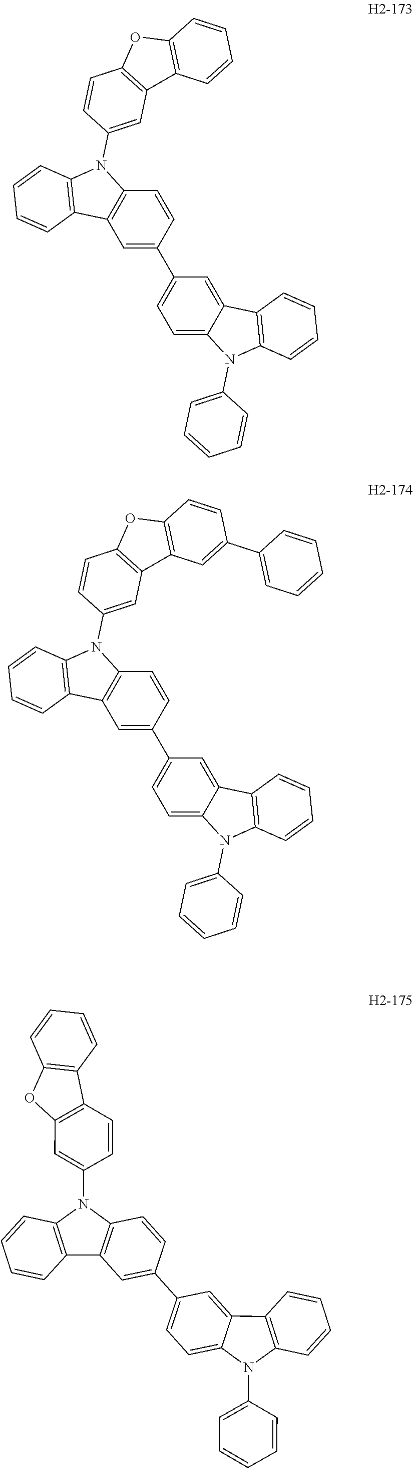 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1]

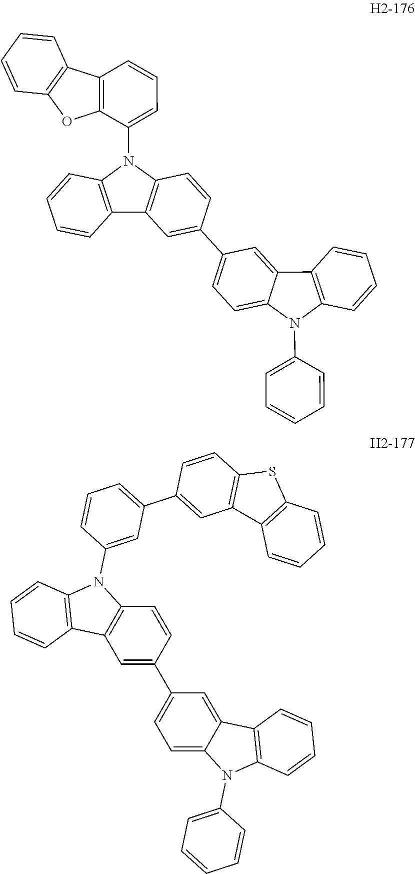 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1]

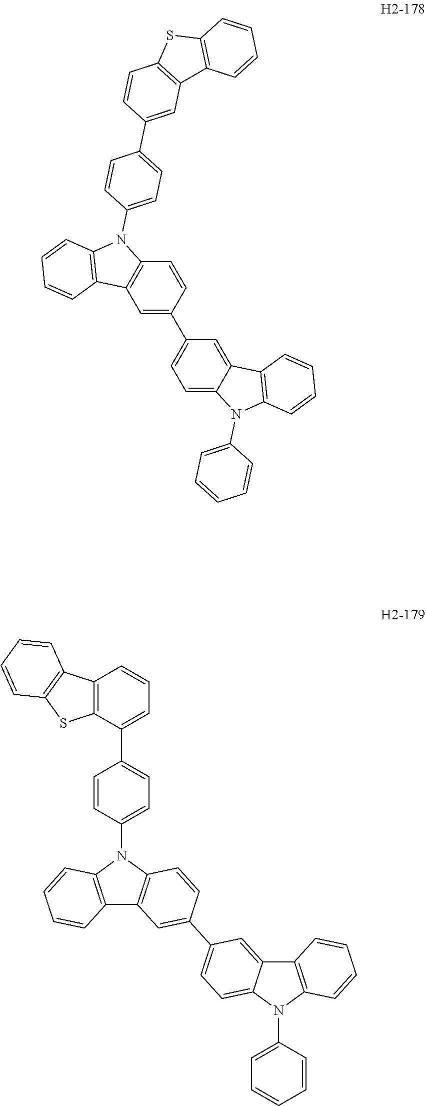 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

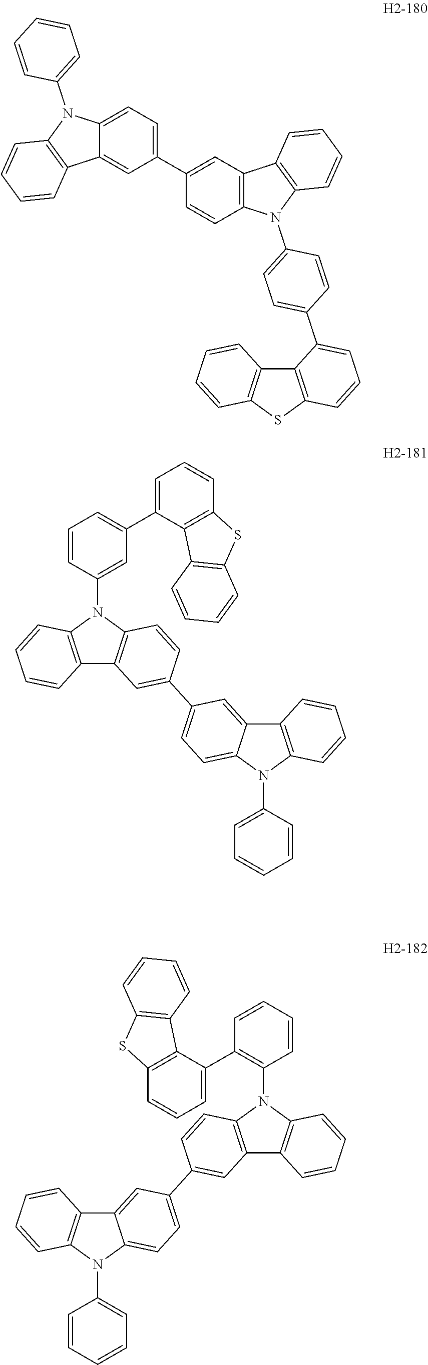 [SMILES: c1ccc(-n2c3ccccc3c3cc(-c4ccc5c(c4)c4ccccc4n5-c4ccc(-c5cccc6sc7ccccc7c56)cc4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1]

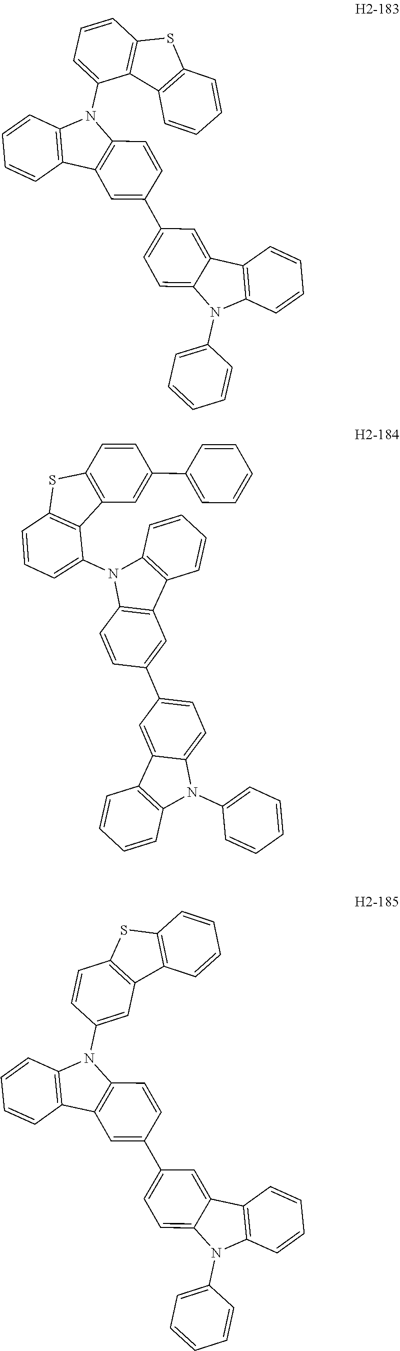

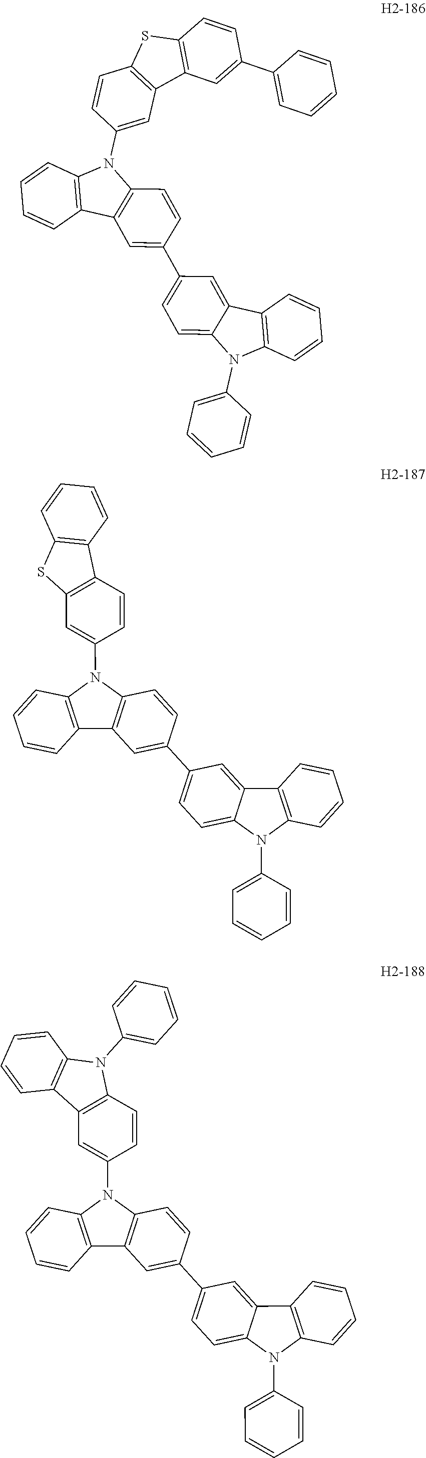 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1]

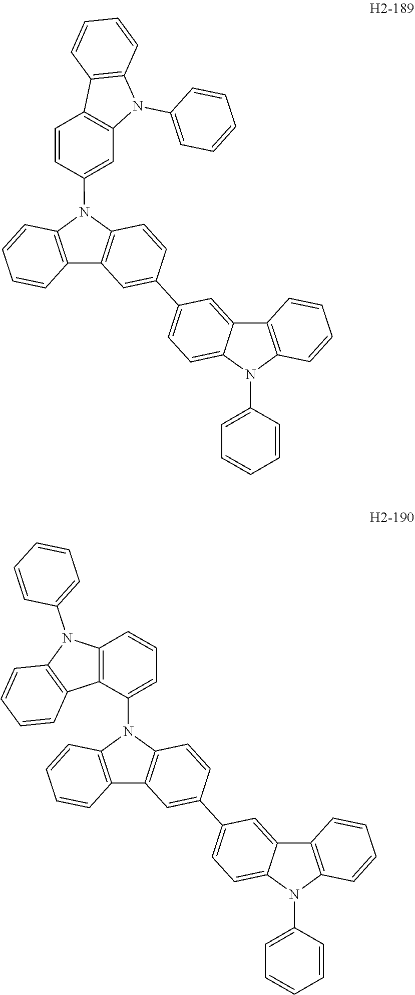 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1]

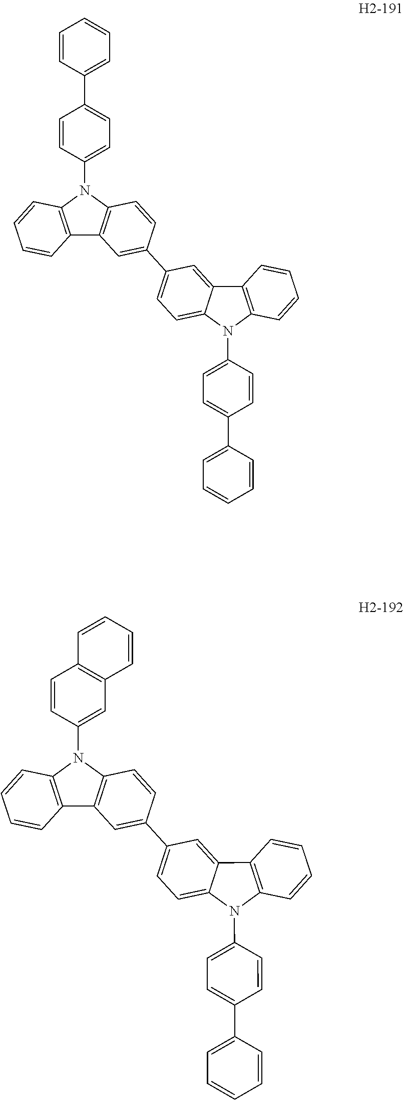 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1]

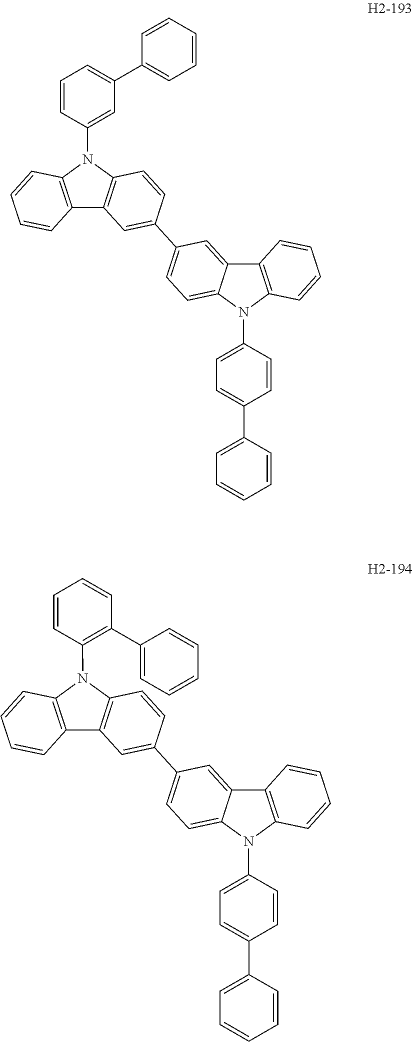 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1]

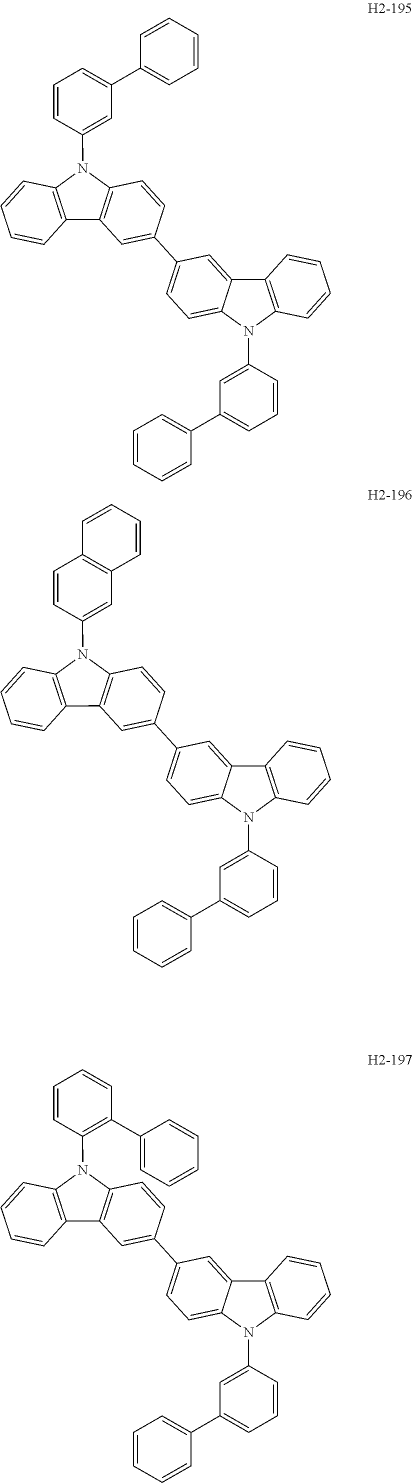

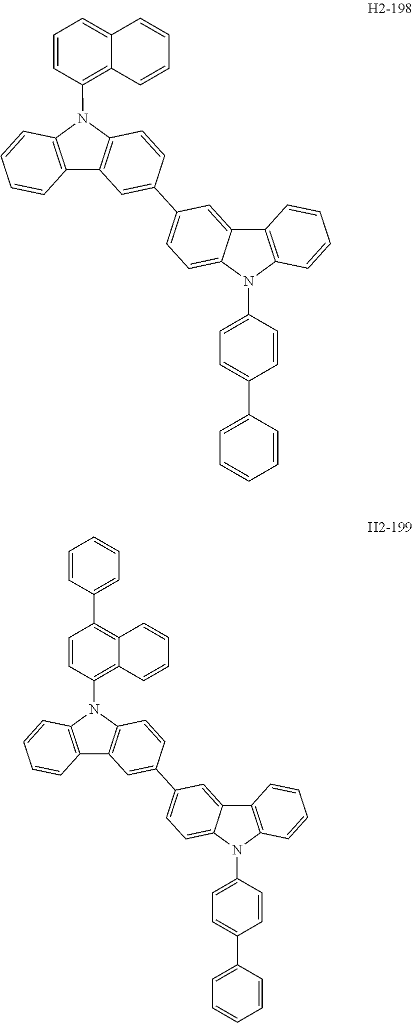 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1]

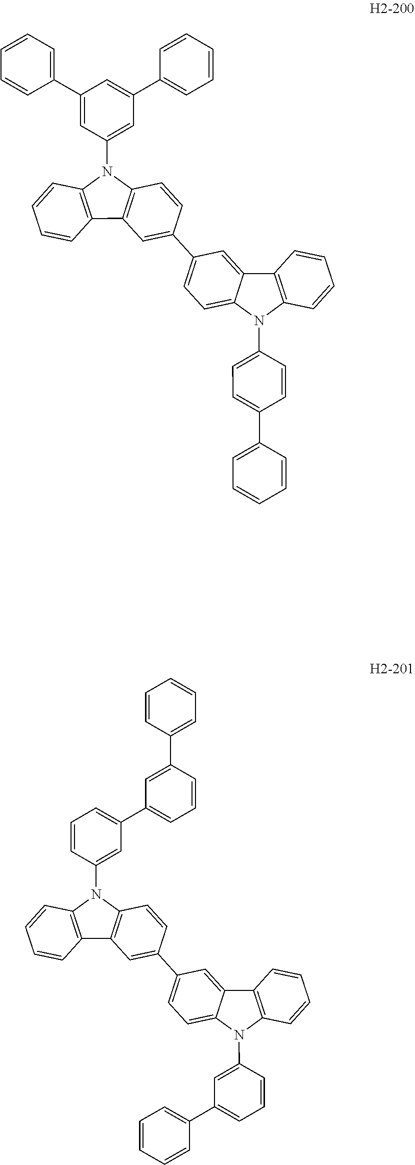 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

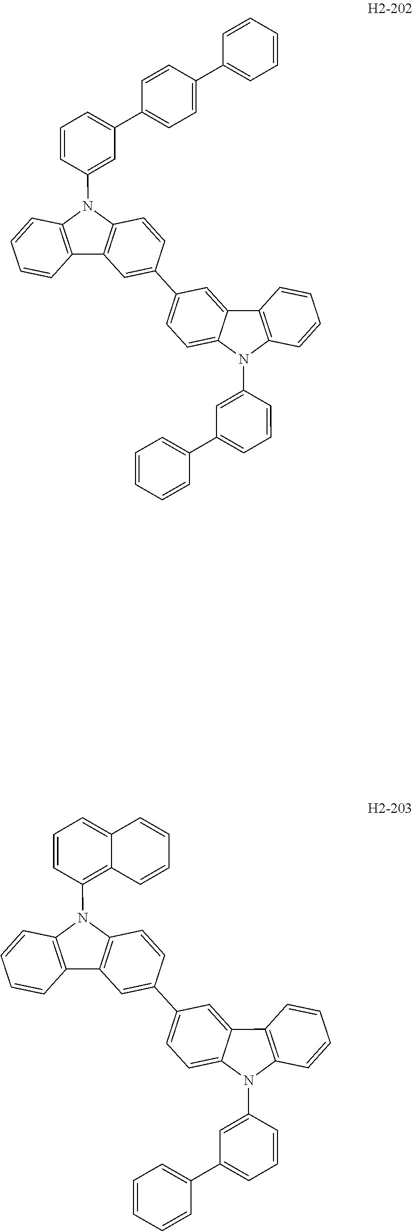 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1]

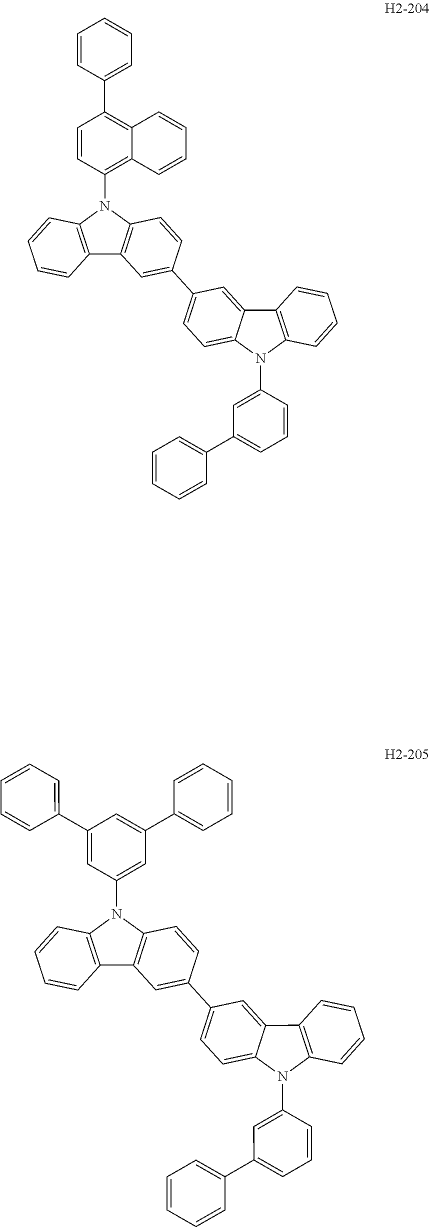 [SMILES: c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

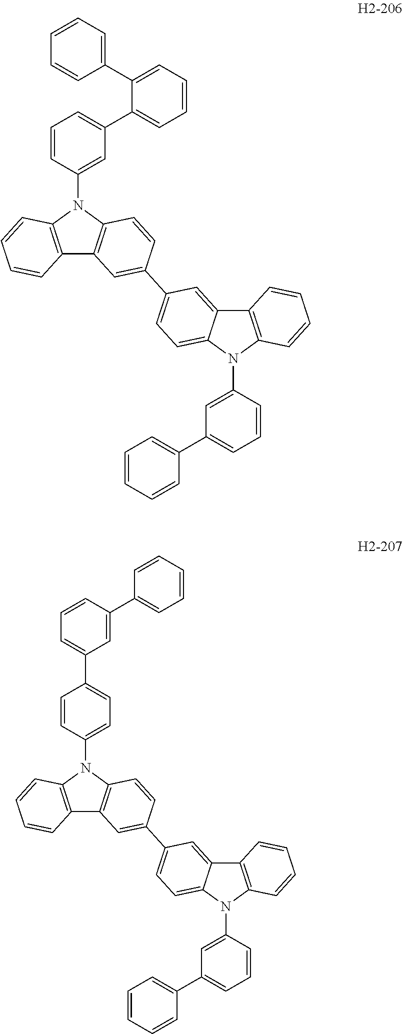 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1]

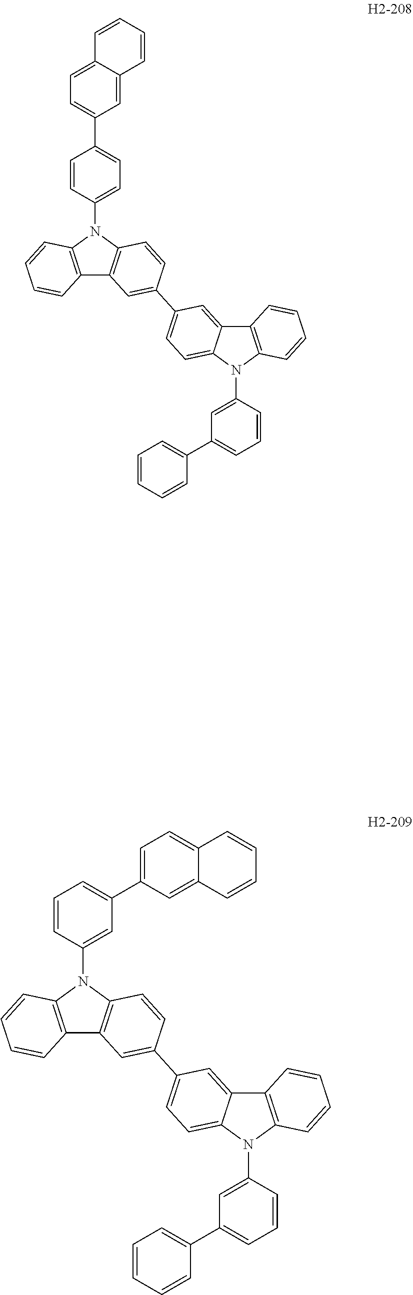 [SMILES: c1ccc(-c2cccc(-n3c4ccccc4c4cc(-c5ccc6c(c5)c5ccccc5n6-c5ccc(-c6ccc7ccccc7c6)cc5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1]

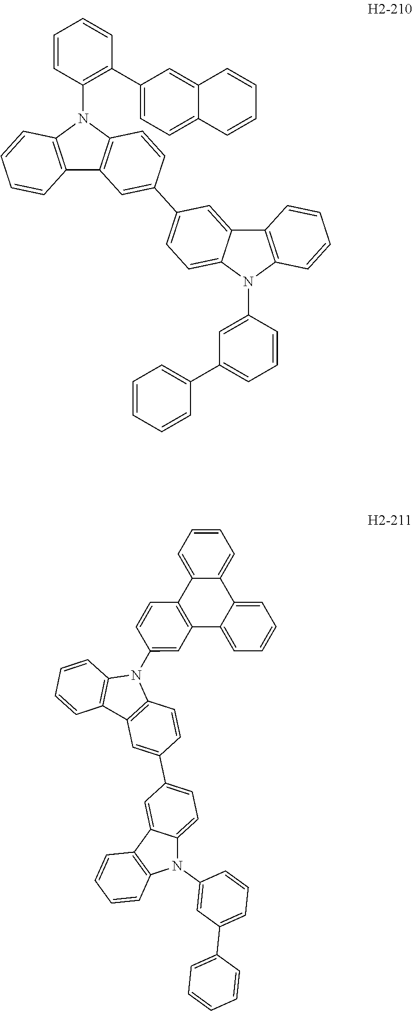 [SMILES: c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1]

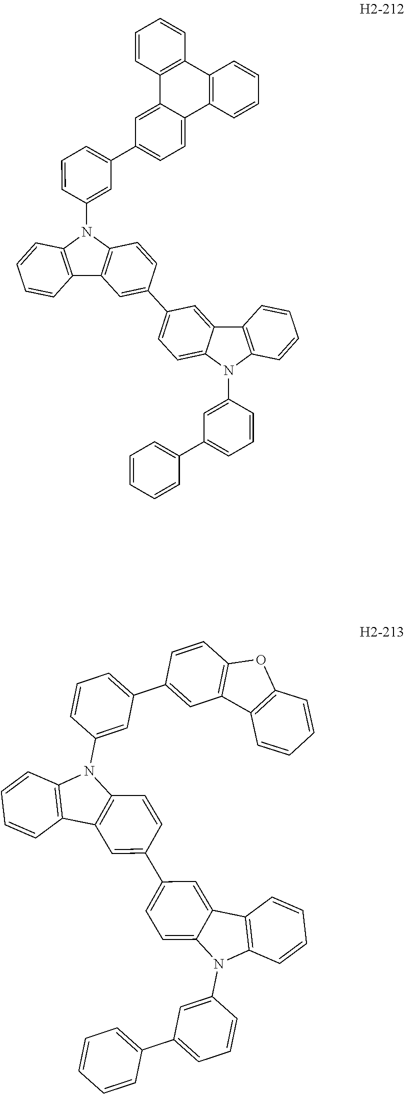 [SMILES: c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1 ; c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1]

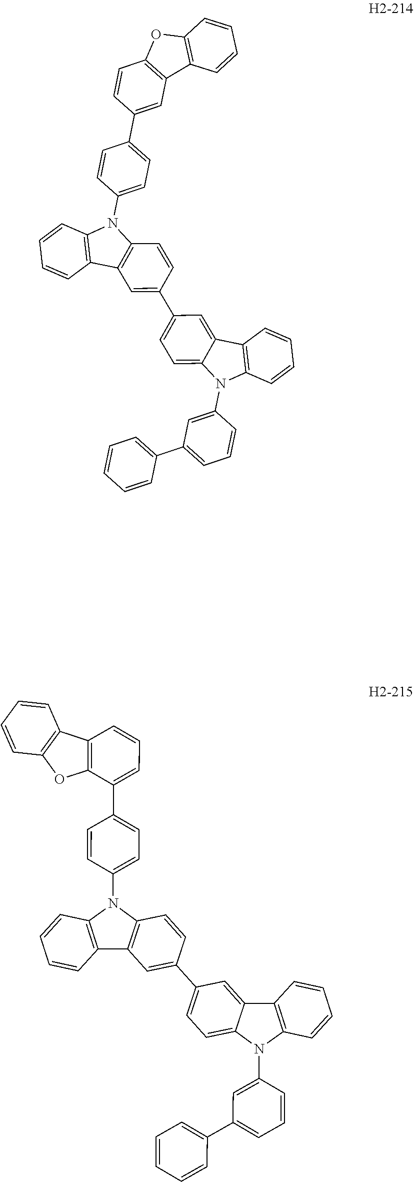 [SMILES: c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

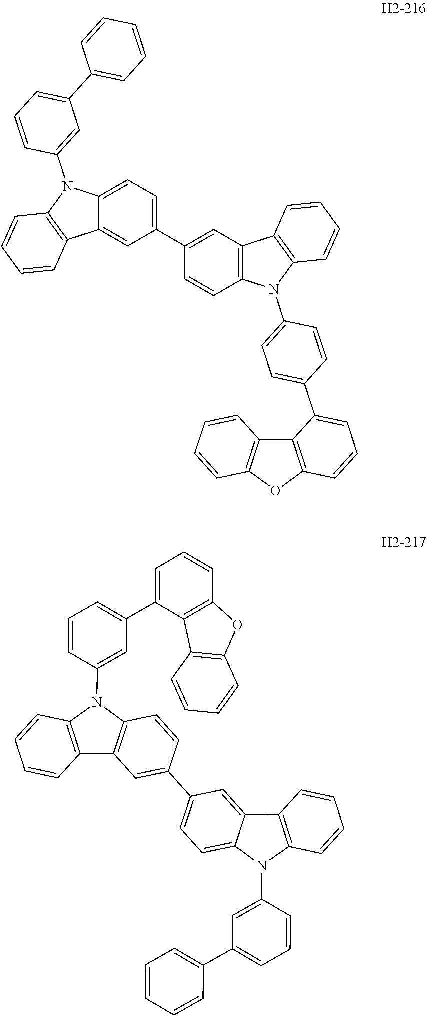 [SMILES: c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1]

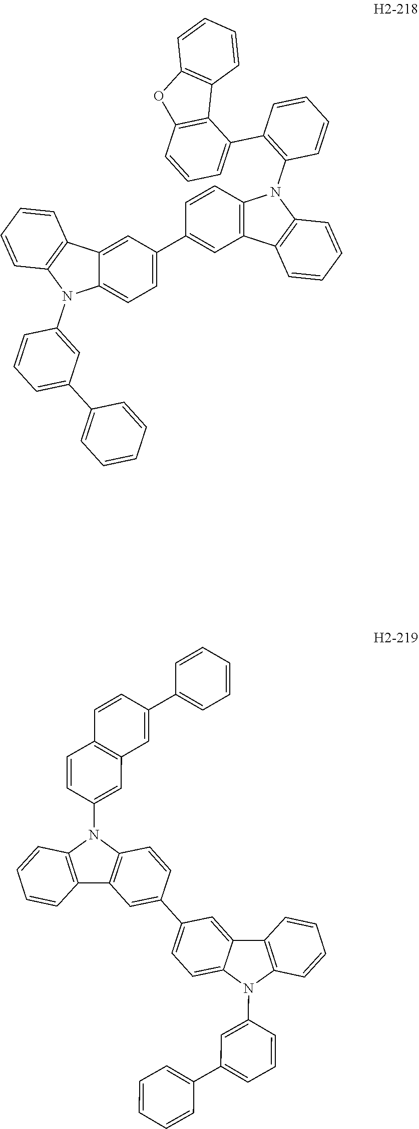 [SMILES: c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1]

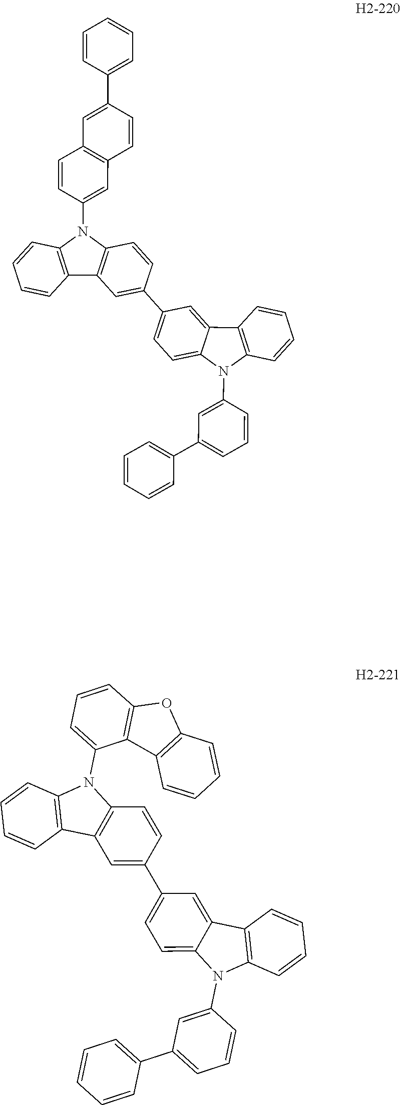 [SMILES: c1ccc(-c2cccc(-n3c4ccccc4c4cc(-c5ccc6c(c5)c5ccccc5n6-c5ccc6cc(-c7ccccc7)ccc6c5)ccc43)c2)cc1 ; c1ccc(-c2cccc(-n3c4ccccc4c4cc(-c5ccc6c(c5)c5ccccc5n6-c5cccc6oc7ccccc7c56)ccc43)c2)cc1]

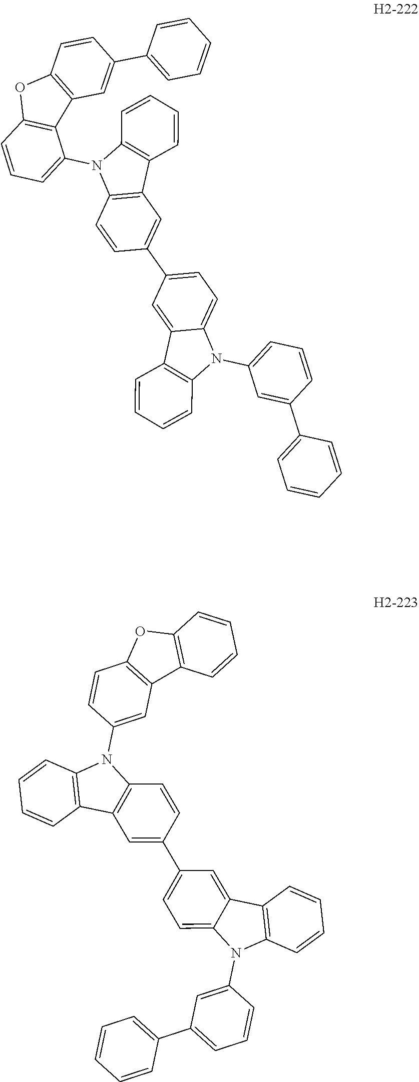 [SMILES: c1ccc(-c2cccc(-n3c4ccccc4c4cc(-c5ccc6c(c5)c5ccccc5n6-c5cccc6oc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

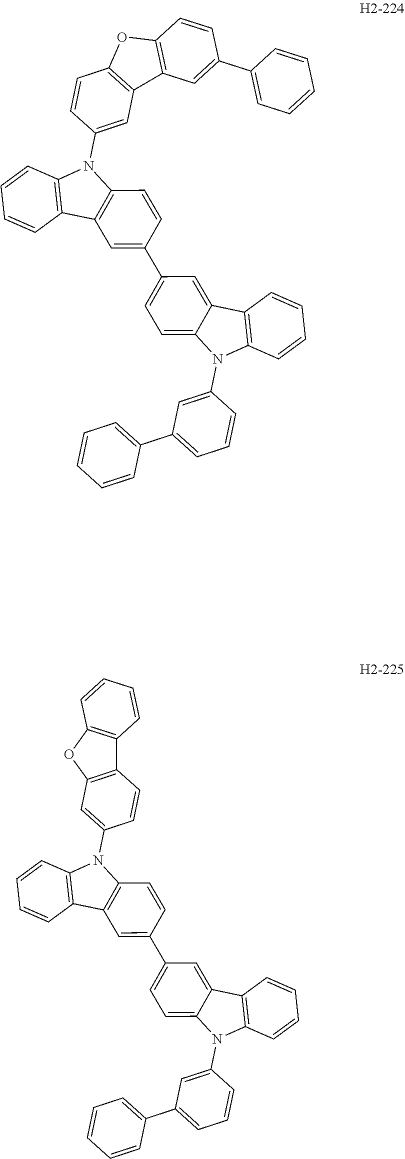 [SMILES: c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)oc5ccccc56)ccc43)c2)cc1]

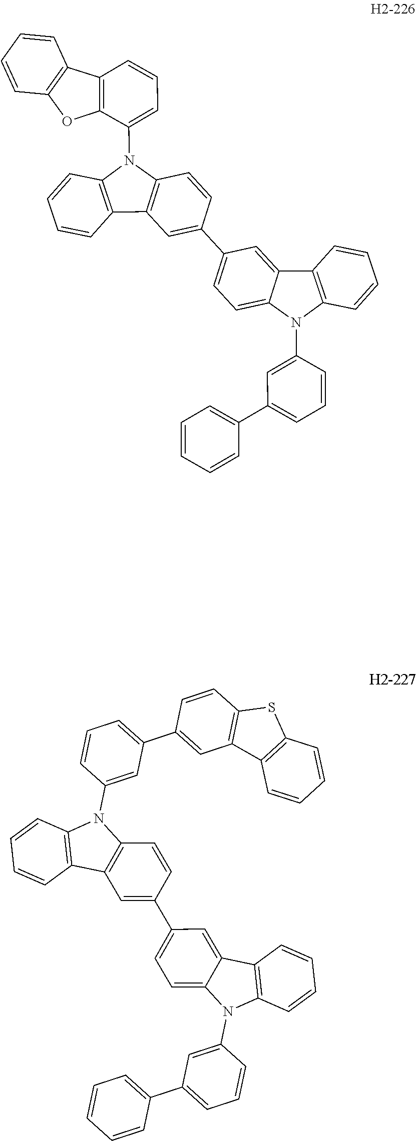 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1]

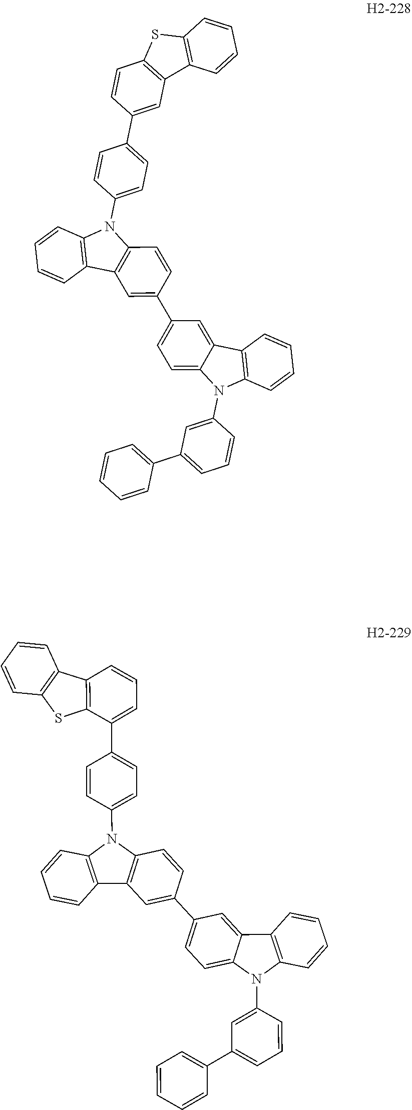 [SMILES: c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1]

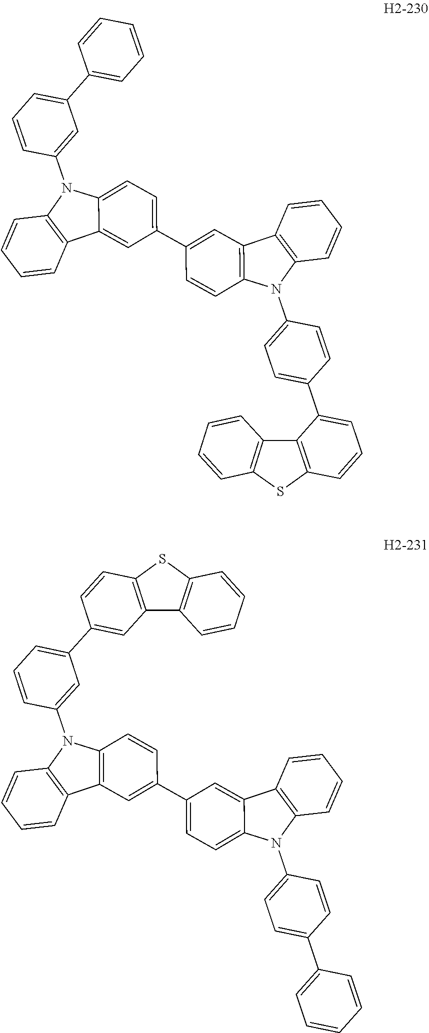 [SMILES: c1ccc(-c2cccc(-n3c4ccccc4c4cc(-c5ccc6c(c5)c5ccccc5n6-c5ccc(-c6cccc7sc8ccccc8c67)cc5)ccc43)c2)cc1 ; c1ccc(-c2ccc(-n3c4ccccc4c4cc(-c5ccc6c(c5)c5ccccc5n6-c5cccc(-c6ccc7sc8ccccc8c7c6)c5)ccc43)cc2)cc1]

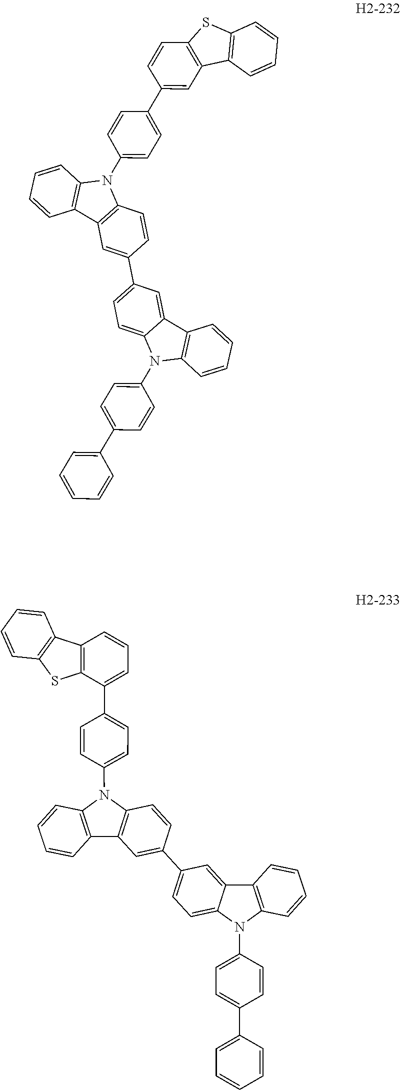 [SMILES: c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1]

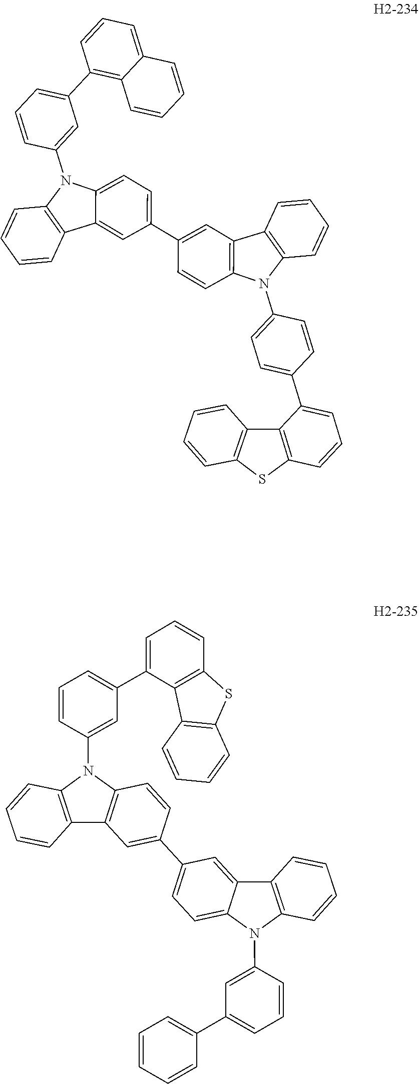 [SMILES: c1cc(-c2cccc3ccccc23)cc(-n2c3ccccc3c3cc(-c4ccc5c(c4)c4ccccc4n5-c4ccc(-c5cccc6sc7ccccc7c56)cc4)ccc32)c1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1]

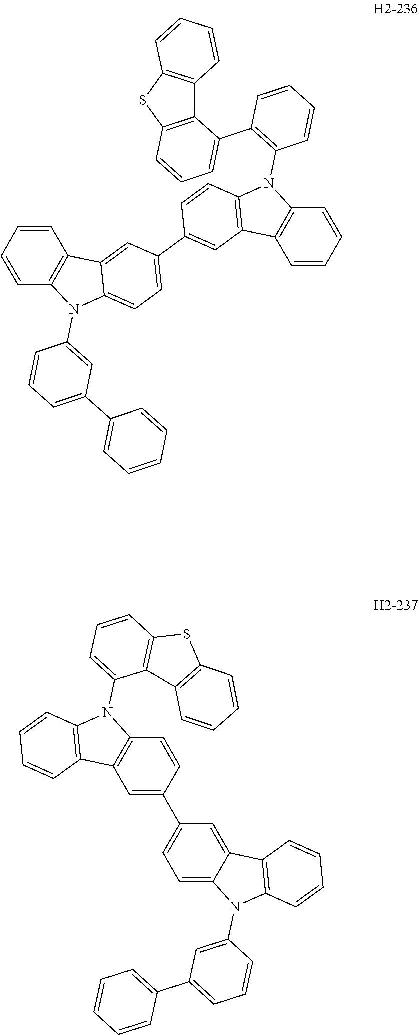 [SMILES: c1ccc(-c2cccc(-n3c4ccccc4c4cc(-c5ccc6c(c5)c5ccccc5n6-c5ccccc5-c5cccc6sc7ccccc7c56)ccc43)c2)cc1 ; c1ccc(-c2cccc(-n3c4ccccc4c4cc(-c5ccc6c(c5)c5ccccc5n6-c5cccc6sc7ccccc7c56)ccc43)c2)cc1]

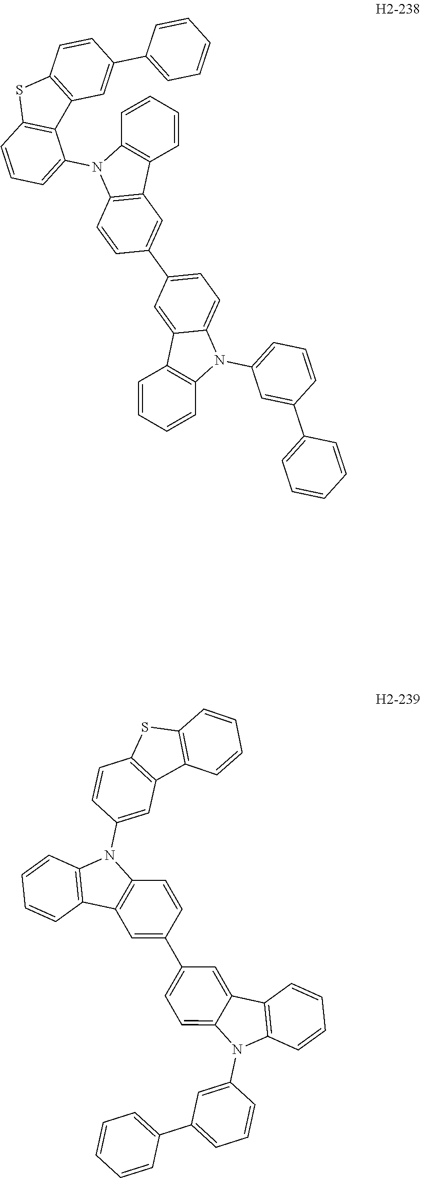 [SMILES: c1ccc(-c2cccc(-n3c4ccccc4c4cc(-c5ccc6c(c5)c5ccccc5n6-c5cccc6sc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

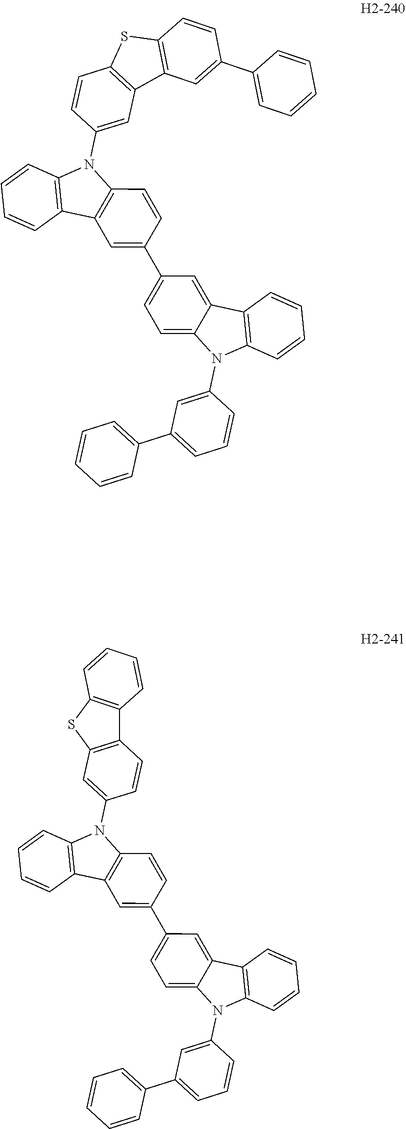 [SMILES: c1ccc(-c2cccc(-n3c4ccccc4c4cc(-c5ccc6c(c5)c5ccccc5n6-c5ccc6sc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)sc5ccccc56)ccc43)c2)cc1]

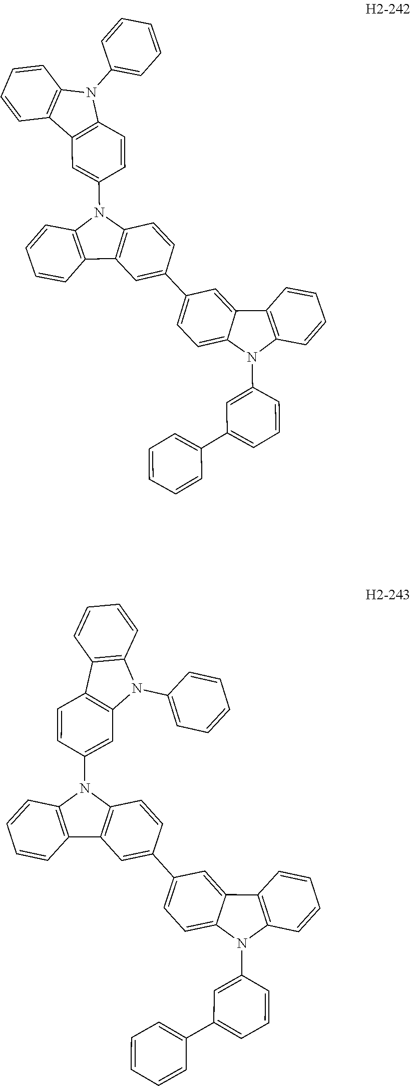 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1]

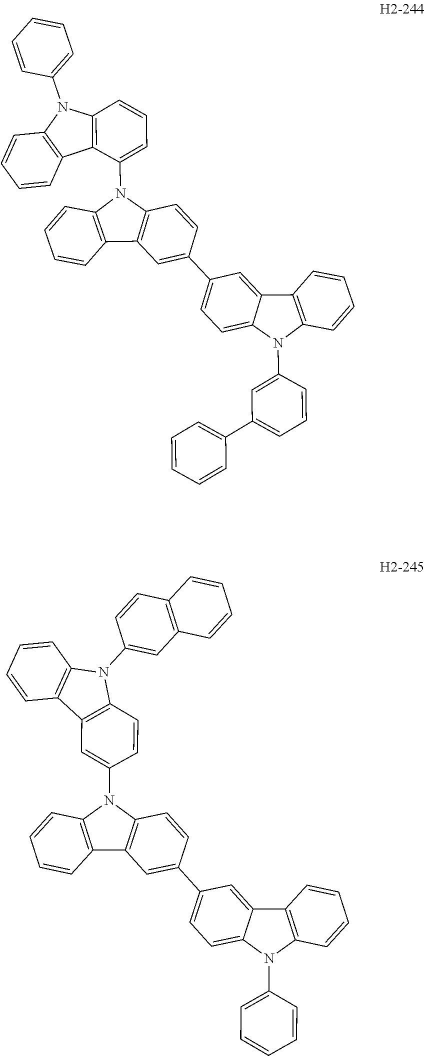 [SMILES: c1ccc(-c2cccc(-n3c4ccccc4c4cc(-c5ccc6c(c5)c5ccccc5n6-c5cccc6c5c5ccccc5n6-c5ccccc5)ccc43)c2)cc1 ; c1ccc(-n2c3ccccc3c3cc(-c4ccc5c(c4)c4ccccc4n5-c4ccc5c(c4)c4ccccc4n5-c4ccc5ccccc5c4)ccc32)cc1]

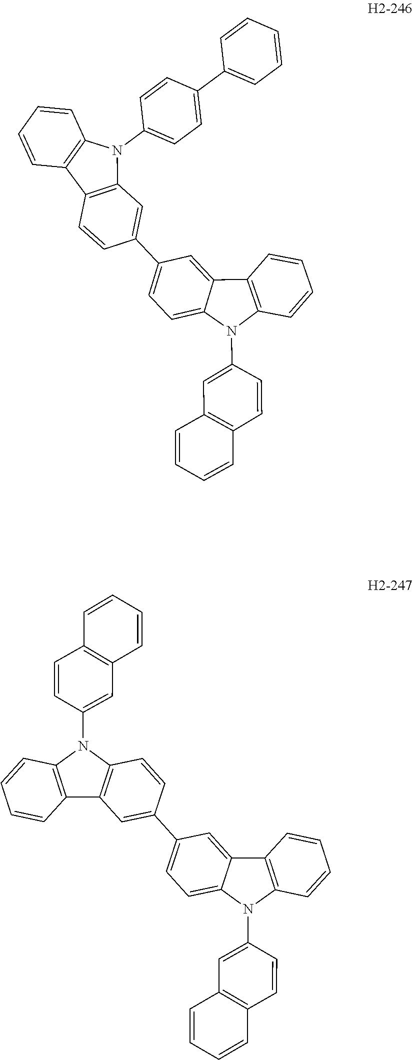 [SMILES: c1ccc(-c2ccc(-n3c4ccccc4c4ccc(-c5ccc6c(c5)c5ccccc5n6-c5ccc6ccccc6c5)cc43)cc2)cc1 ; c1ccc2cc(-n3c4ccccc4c4cc(-c5ccc6c(c5)c5ccccc5n6-c5ccc6ccccc6c5)ccc43)ccc2c1]

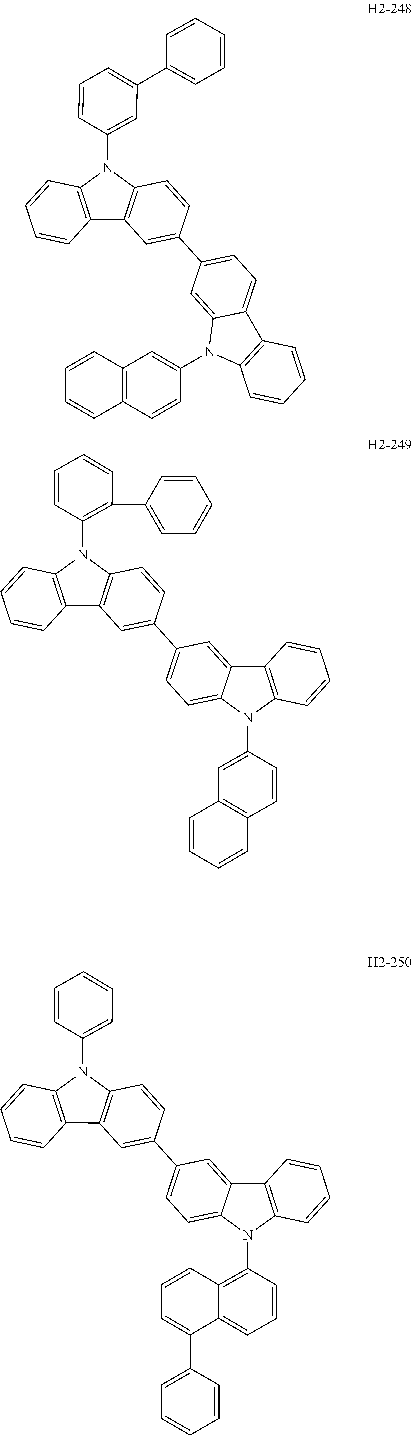

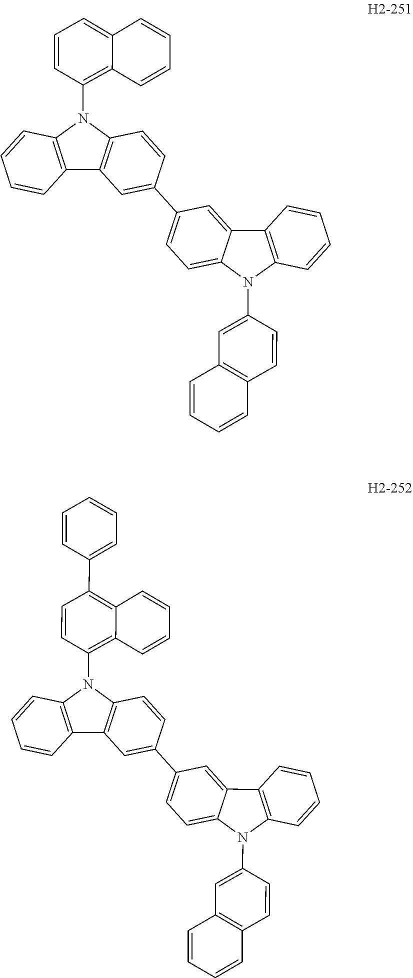 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1]

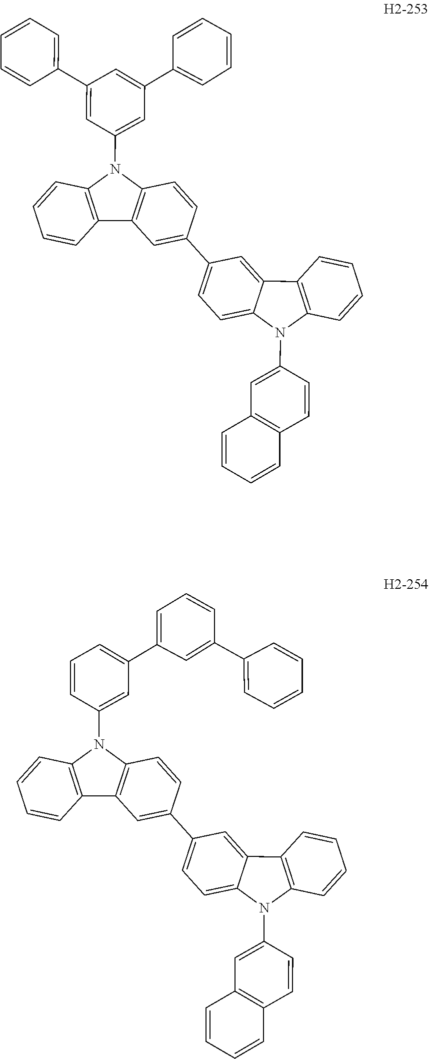 [SMILES: c1ccc(-c2cc(-c3ccccc3)cc(-n3c4ccccc4c4cc(-c5ccc6c(c5)c5ccccc5n6-c5ccc6ccccc6c5)ccc43)c2)cc1 ; c1ccc(-c2cccc(-c3cccc(-n4c5ccccc5c5cc(-c6ccc7c(c6)c6ccccc6n7-c6ccc7ccccc7c6)ccc54)c3)c2)cc1]

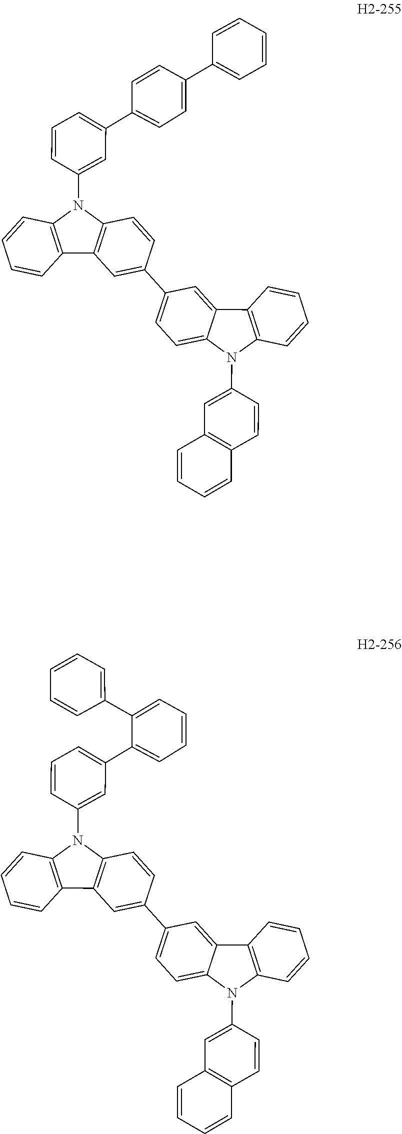 [SMILES: c1ccc(-c2ccc(-c3cccc(-n4c5ccccc5c5cc(-c6ccc7c(c6)c6ccccc6n7-c6ccc7ccccc7c6)ccc54)c3)cc2)cc1 ; c1ccc(-c2ccccc2-c2cccc(-n3c4ccccc4c4cc(-c5ccc6c(c5)c5ccccc5n6-c5ccc6ccccc6c5)ccc43)c2)cc1]

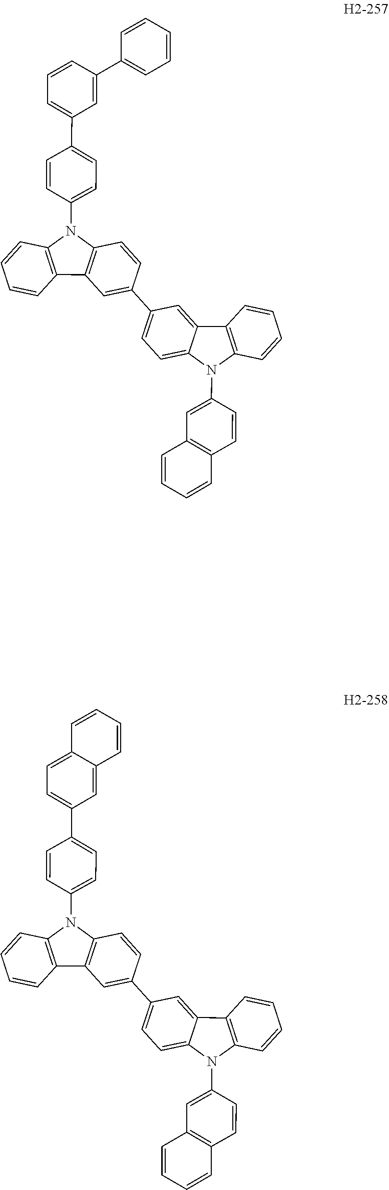 [SMILES: c1ccc(-c2cccc(-c3ccc(-n4c5ccccc5c5cc(-c6ccc7c(c6)c6ccccc6n7-c6ccc7ccccc7c6)ccc54)cc3)c2)cc1 ; c1ccc2cc(-c3ccc(-n4c5ccccc5c5cc(-c6ccc7c(c6)c6ccccc6n7-c6ccc7ccccc7c6)ccc54)cc3)ccc2c1]

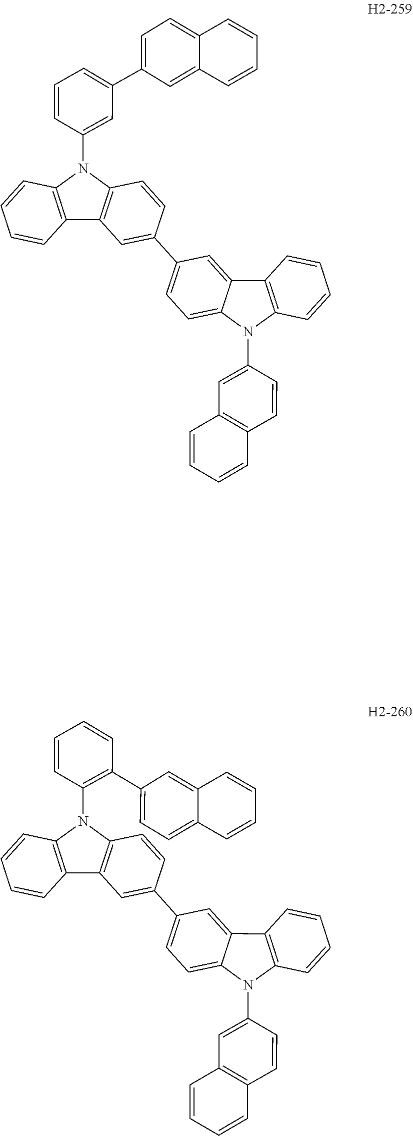 [SMILES: c1cc(-c2ccc3ccccc3c2)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2ccc3ccccc3c2)c1]

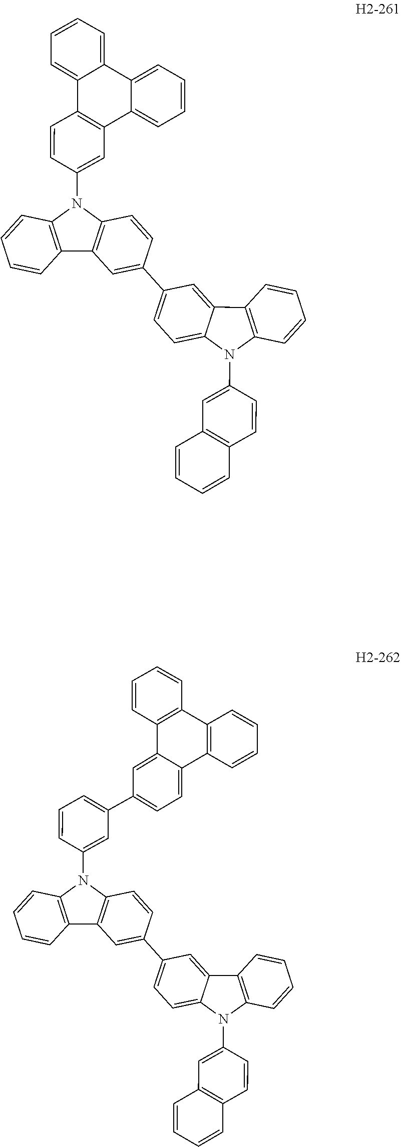 [SMILES: c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1]

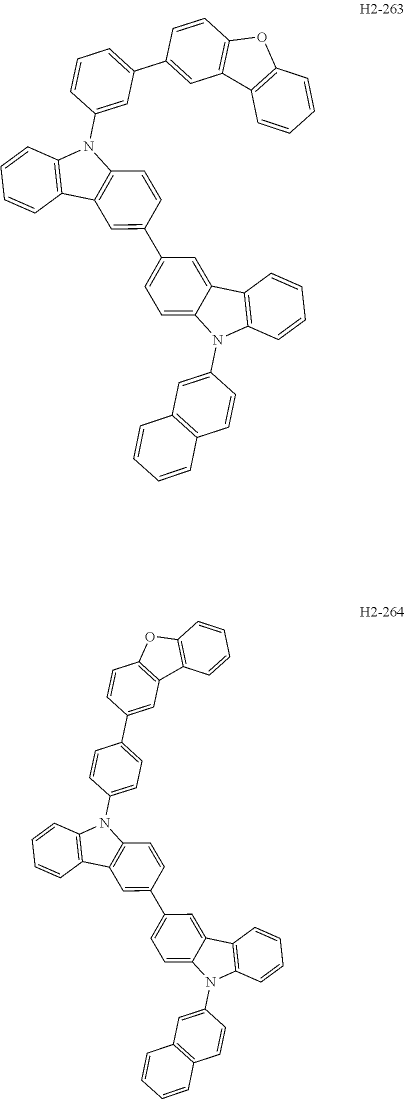 [SMILES: c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1]

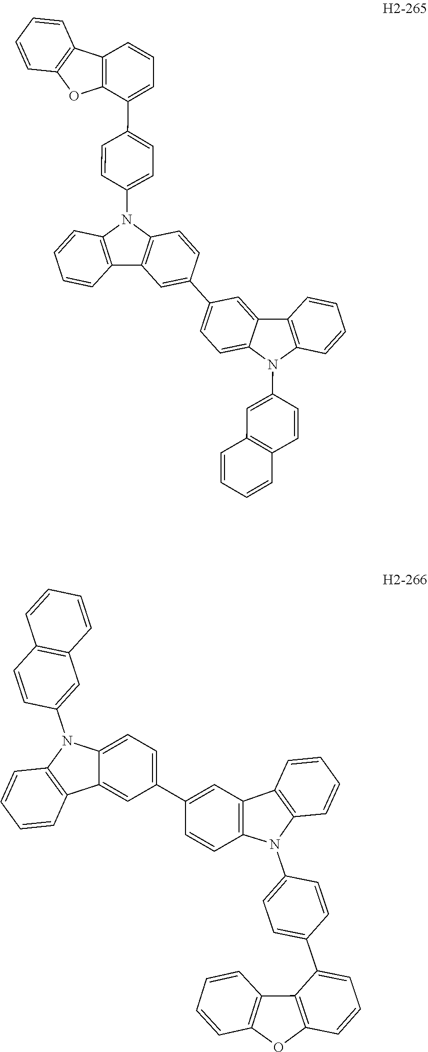 [SMILES: c1ccc2cc(-n3c4ccccc4c4cc(-c5ccc6c(c5)c5ccccc5n6-c5ccc(-c6cccc7c6oc6ccccc67)cc5)ccc43)ccc2c1 ; c1ccc2cc(-n3c4ccccc4c4cc(-c5ccc6c(c5)c5ccccc5n6-c5ccc(-c6cccc7oc8ccccc8c67)cc5)ccc43)ccc2c1]

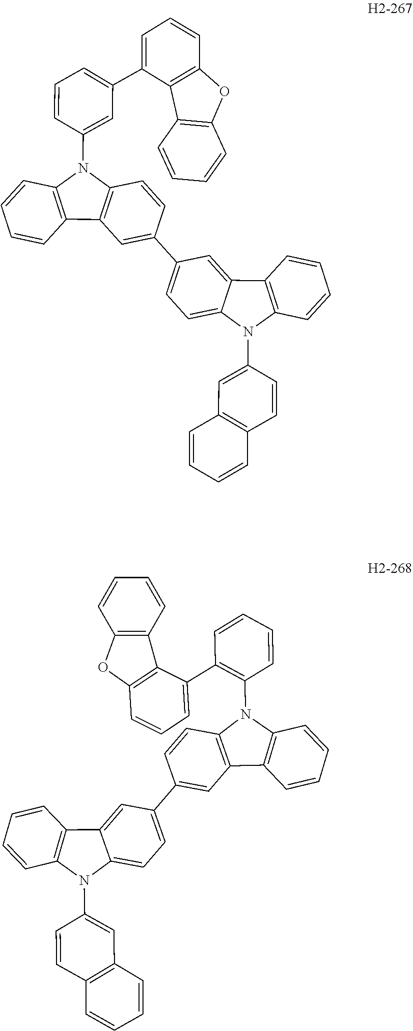 [SMILES: c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1]

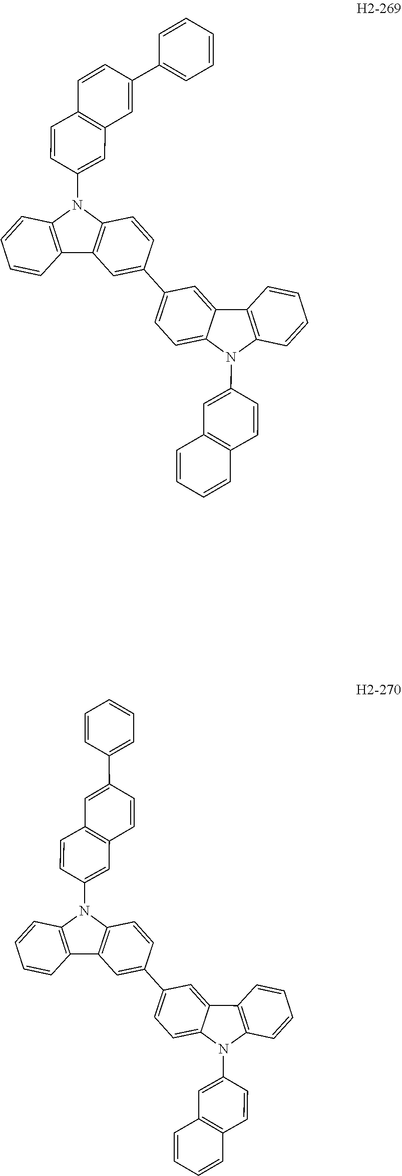 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1]

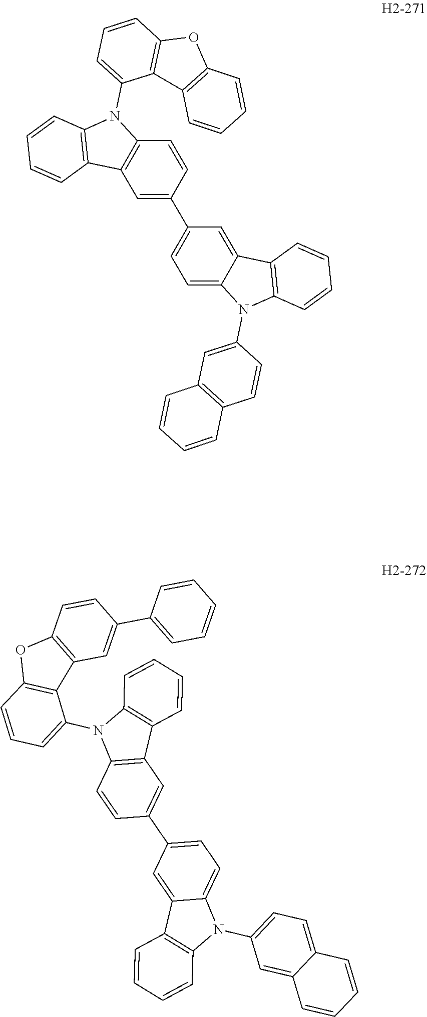 [SMILES: c1ccc2cc(-n3c4ccccc4c4cc(-c5ccc6c(c5)c5ccccc5n6-c5cccc6oc7ccccc7c56)ccc43)ccc2c1 ; c1ccc(-c2ccc3oc4cccc(-n5c6ccccc6c6cc(-c7ccc8c(c7)c7ccccc7n8-c7ccc8ccccc8c7)ccc65)c4c3c2)cc1]

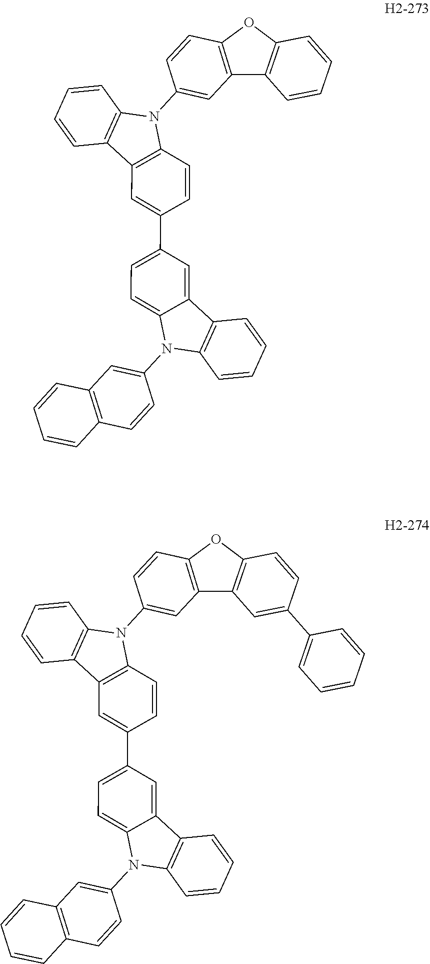 [SMILES: c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1]

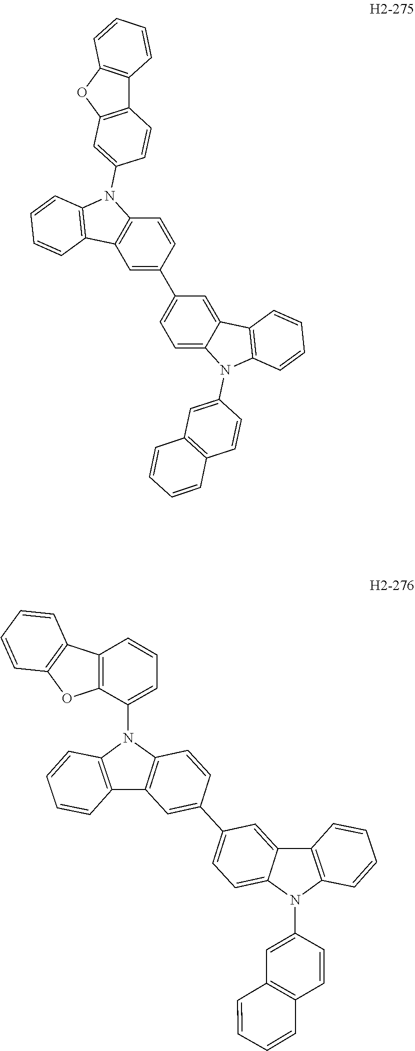 [SMILES: c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1]

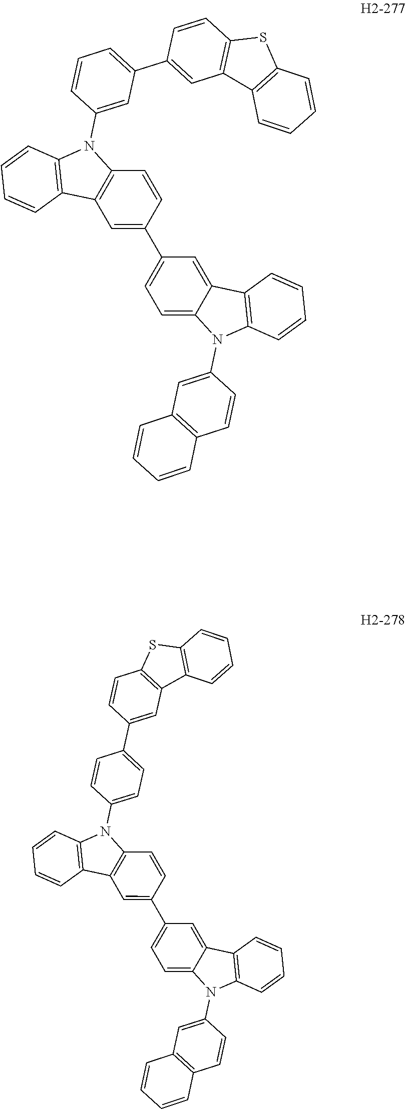 [SMILES: c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1]

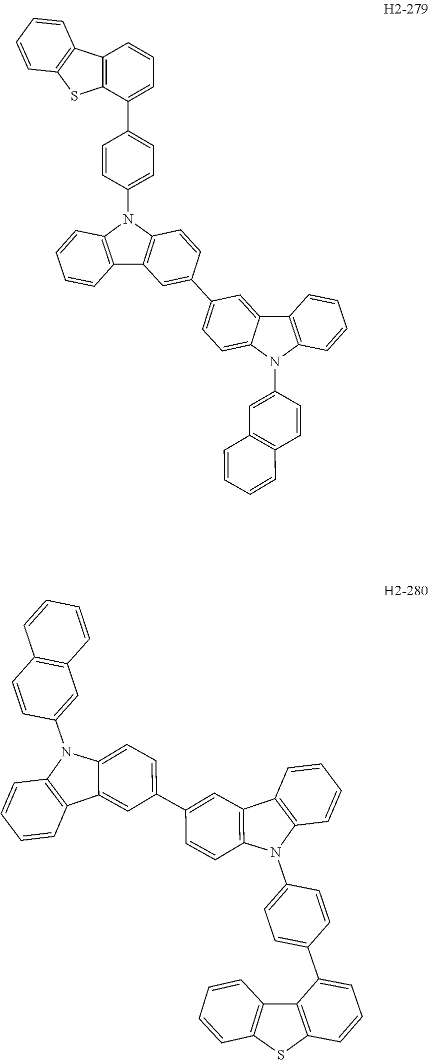 [SMILES: c1ccc2cc(-n3c4ccccc4c4cc(-c5ccc6c(c5)c5ccccc5n6-c5ccc(-c6cccc7c6sc6ccccc67)cc5)ccc43)ccc2c1 ; c1ccc2cc(-n3c4ccccc4c4cc(-c5ccc6c(c5)c5ccccc5n6-c5ccc(-c6cccc7sc8ccccc8c67)cc5)ccc43)ccc2c1]

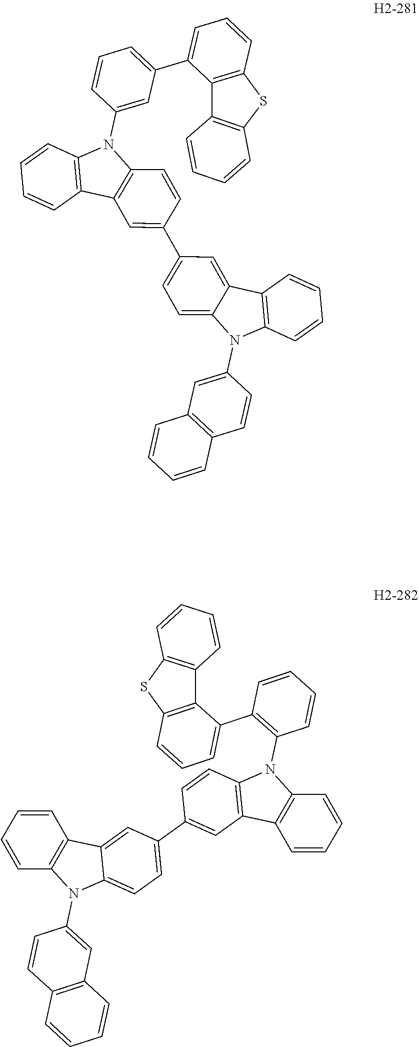 [SMILES: c1cc(-c2cccc3sc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3sc4ccccc4c23)c1]

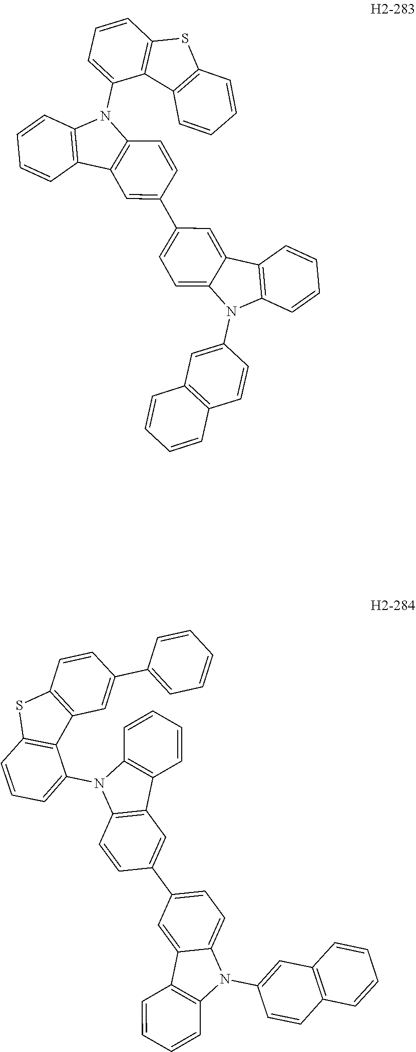 [SMILES: c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1]

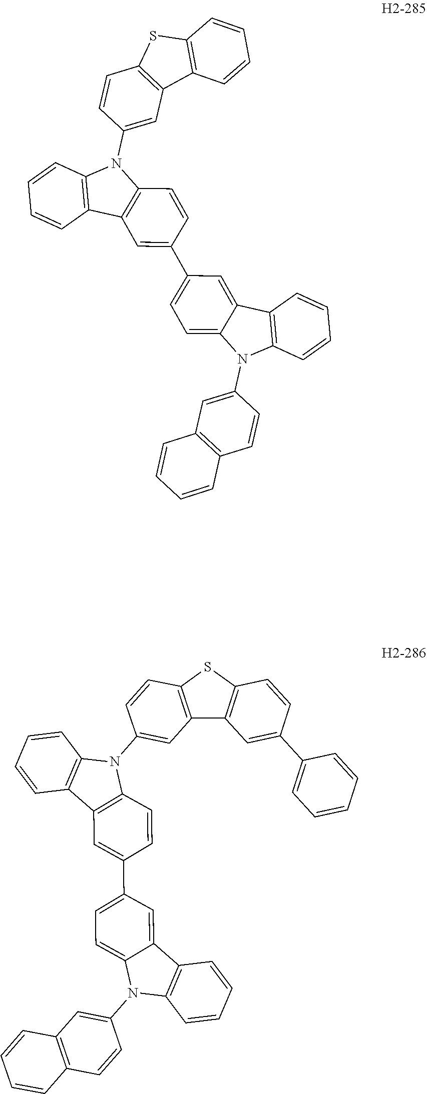 [SMILES: c1ccc2cc(-n3c4ccccc4c4cc(-c5ccc6c(c5)c5ccccc5n6-c5ccc6sc7ccccc7c6c5)ccc43)ccc2c1 ; c1ccc(-c2ccc3sc4ccc(-n5c6ccccc6c6cc(-c7ccc8c(c7)c7ccccc7n8-c7ccc8ccccc8c7)ccc65)cc4c3c2)cc1]

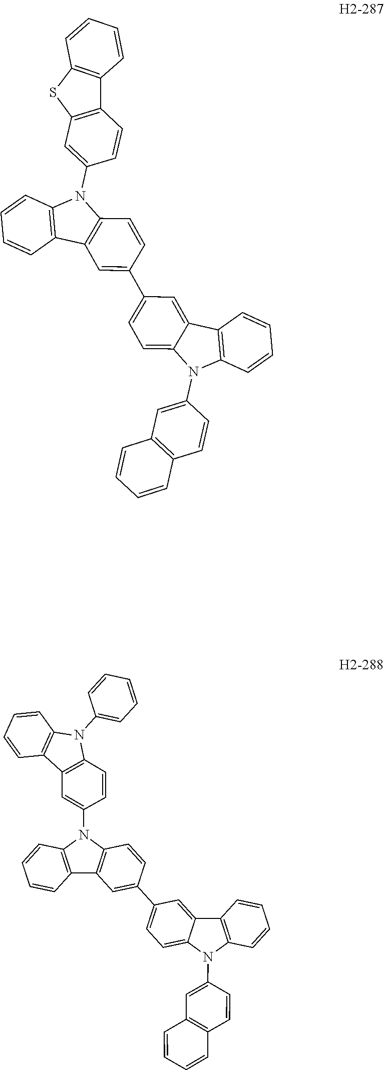 [SMILES: c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1]

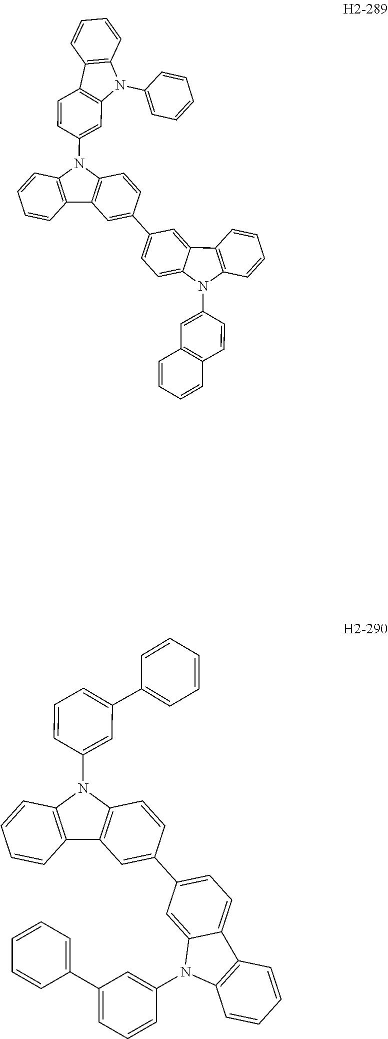 [SMILES: c1ccc(-n2c3ccccc3c3ccc(-n4c5ccccc5c5cc(-c6ccc7c(c6)c6ccccc6n7-c6ccc7ccccc7c6)ccc54)cc32)cc1 ; c1ccc(-c2cccc(-n3c4ccccc4c4cc(-c5ccc6c7ccccc7n(-c7cccc(-c8ccccc8)c7)c6c5)ccc43)c2)cc1]

In the compounds above, Dn means that n number of hydrogens is replaced with deuterium, wherein n is an integer of 1 or more and the upper limit of n is determined according to the number of hydrogens that may be substituted for each compound.

The compound represented by Formula 2 according to the present disclosure can be prepared by a synthetic method known to one skilled in the art, for example, may be prepared by referring to the following Reaction Scheme 5, but is not limited thereto.

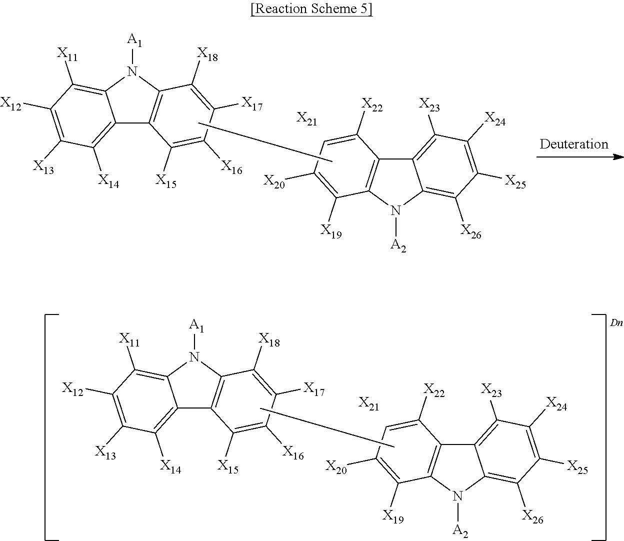

In reaction scheme 5, A₁, A₂, X₁₁ to X₂₆, and n are as defined in Formula 2, and Dn means that n number of hydrogens is replaced with deuterium.

In addition, the deuterated compound of Formula 2 can be prepared using a deuterated precursor material in a similar manner, or more generally can be prepared by treating a non-deuterated compound with a deuterated solvent, D6-benzene in the presence of a Lewis acid H/D exchange catalyst such as aluminium trichloride or ethyl aluminium chloride. In addition, the degree of deuteration can be controlled by varying reaction conditions such as reaction temperature. For example, the number of deuterium in Formula 2 can be adjusted by controlling the reaction temperature and time, the equivalent of acid, etc.

According to another embodiment of the present disclosure, the present disclosure provides an organic electroluminescent compound represented by the following Formula 1′.

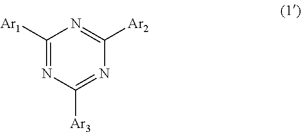

In Formula 1′,
Ar₁ and Ar₂ each independently represent, a substituted or unsubstituted (C6-C30)aryl or a substituted or unsubstituted (3- to 30-membered)heteroaryl;
Ar₃ represents a carbazole group represented by the following Formula 1-1;

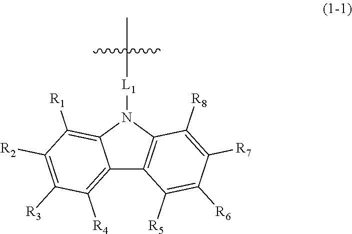

In Formula 1-1,
L₁ represents a single bond or (C6-C30)arylene unsubstituted or substituted with deuterium;
R₁ to R₈ each independently represent, hydrogen, deuterium, or (C6-C30)aryl unsubstituted or substituted with deuterium, (C1-C30)alkyl, (C6-C30)aryl, or a combination thereof; provided that at least one of R₁ to R₄ is represented by the following Formula 1-11;

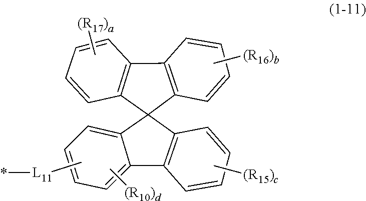

In Formula 1-11,
L₁₁ represents a single bond or (C6-C30)arylene unsubstituted or substituted with deuterium;
R₁₀ and R₁₅ to R₁₇ each independently represent, hydrogen, deuterium, or (C6-C30)aryl unsubstituted or substituted with deuterium; and
a to c each independently represent, an integer of 1 to 4; d is an integer of 1 to 3; when a to d are 2 or more, each of R₁₀ and each of R₁₅ to each of R₁₇ may be the same or different;
provided that the following compound is excluded from Formula 1′.

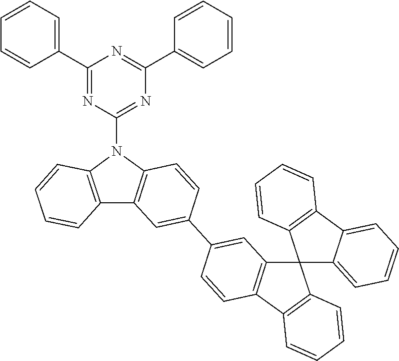 [SMILES: c1ccc(-c2nc(-c3ccccc3)nc(-n3c4ccccc4c4cc(-c5ccc6c(c5)C5(c7ccccc7-c7ccccc75)c5ccccc5-6)ccc43)n2)cc1]

In one embodiment, Ar₁ and Ar₂ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, Ar₁ and Ar₂ each independently may be a substituted or unsubstituted phenyl, a substituted or unsubstituted p-biphenyl, a substituted or unsubstituted m-biphenyl, a substituted or unsubstituted o-biphenyl, a substituted or unsubstituted naphthyl, a substituted or unsubstituted p-terphenyl, a substituted or unsubstituted m-terphenyl, or a substituted or unsubstituted dibenzofuranyl.

In one embodiment, Ar₃ may be a carbazole group represented by Formula 1-1.

In one embodiment, L₁₁ may be a single bond.

In one embodiment, R₁₀ and R₁₅ to R₁₇ each independently may be hydrogen, deuterium, or (C6-C25)aryl unsubstituted or substituted with deuterium, preferably hydrogen, deuterium, or (C6-C18)aryl unsubstituted or substituted with deuterium, more preferably hydrogen or an unsubstituted (C6-C18)aryl. For example, R₁₀ and R₁₅ to R₁₇ each independently may be hydrogen or an unsubstituted phenyl.

According to one embodiment, the Formula 1′ is a compound that satisfies the following <Condition 1> or <Condition 2>:
<Condition 1> Ar₁ and Ar₂ are asymmetric to each other.
<Condition 2> L₁ represents (C6-C30)arylene unsubstituted or substituted with deuterium.

According to one embodiment, the organic electroluminescent compound represented by Formula 1′ may be more specifically illustrated by the following compounds, but is not limited thereto.

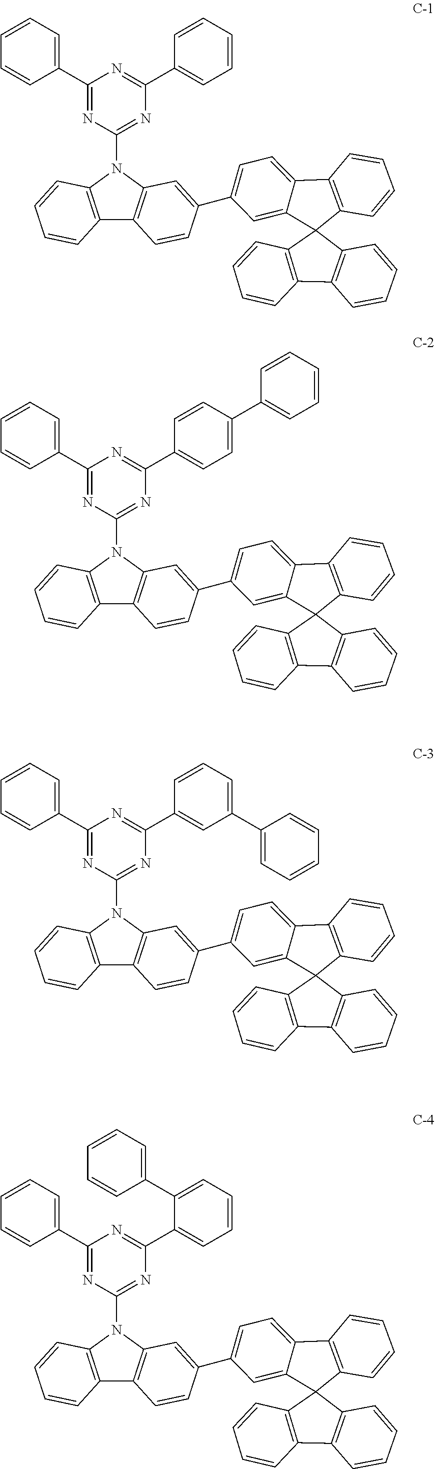 [SMILES: c1ccc(-c2nc(-c3ccccc3)nc(-n3c4ccccc4c4ccc(-c5ccc6c(c5)C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2nc(-c3ccccc3-c3ccccc3)nc(-n3c4ccccc4c4ccc(-c5ccc6c(c5)C5(c7ccccc7-c7ccccc75)c5ccccc5-6)cc43)n2)cc1]

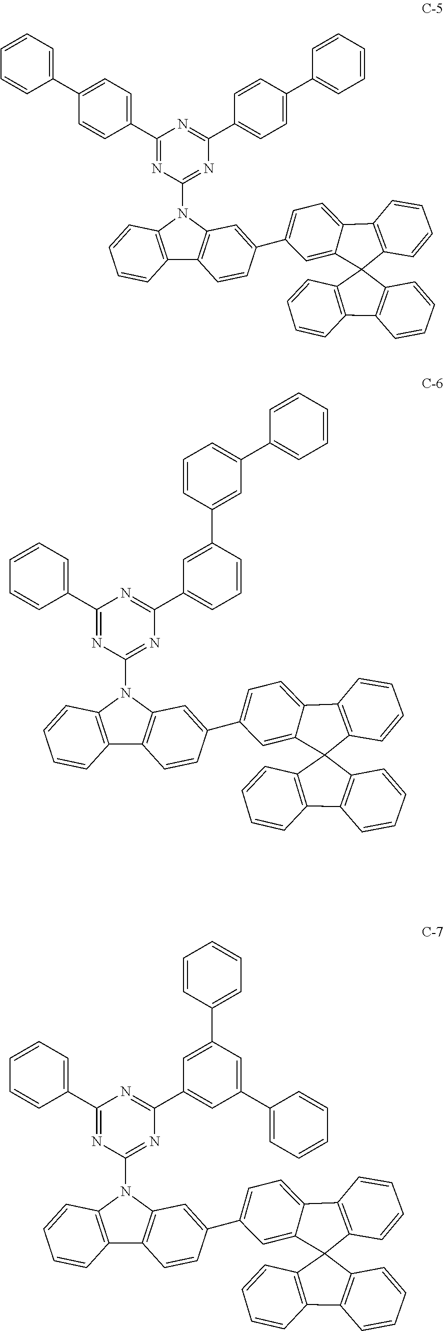 [SMILES: c1ccc(-c2ccc(-c3nc(-c4ccc(-c5ccccc5)cc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)c2)cc1 ; c1ccc(-c2cc(-c3ccccc3)cc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1]

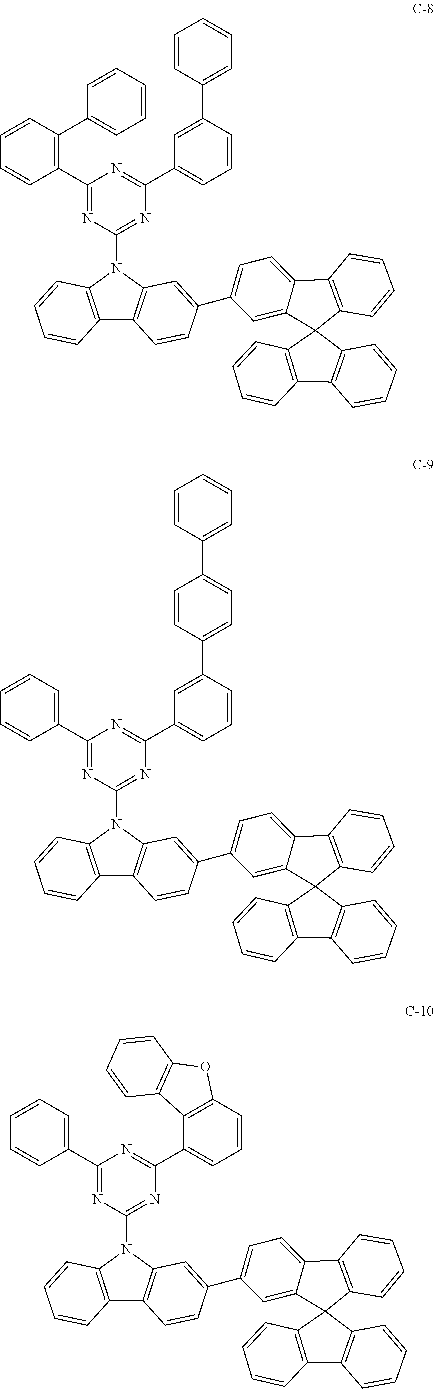 [SMILES: c1ccc(-c2cccc(-c3nc(-c4ccccc4-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2ccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)cc2)cc1 ; c1ccc(-c2nc(-c3cccc4oc5ccccc5c34)nc(-n3c4ccccc4c4ccc(-c5ccc6c(c5)C5(c7ccccc7-c7ccccc75)c5ccccc5-6)cc43)n2)cc1]

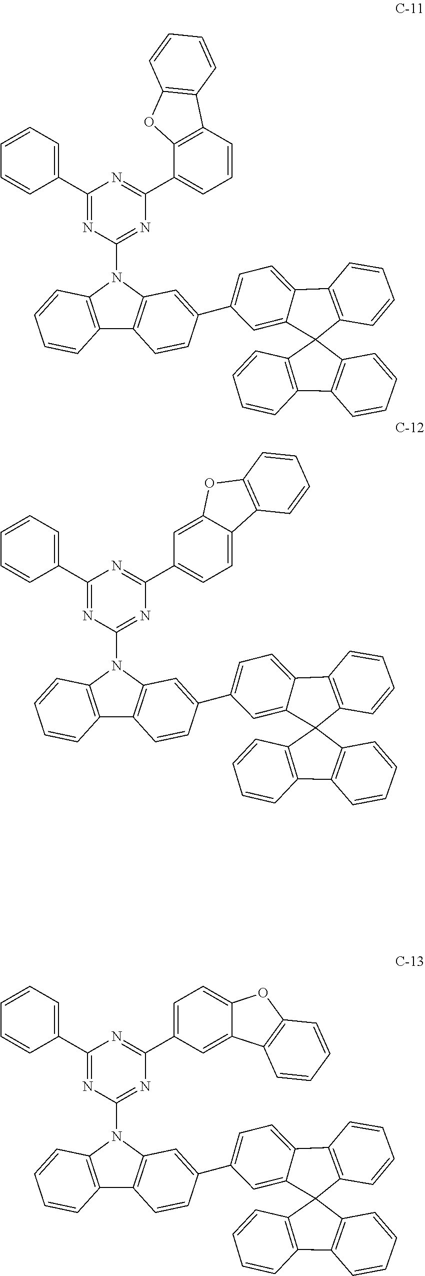

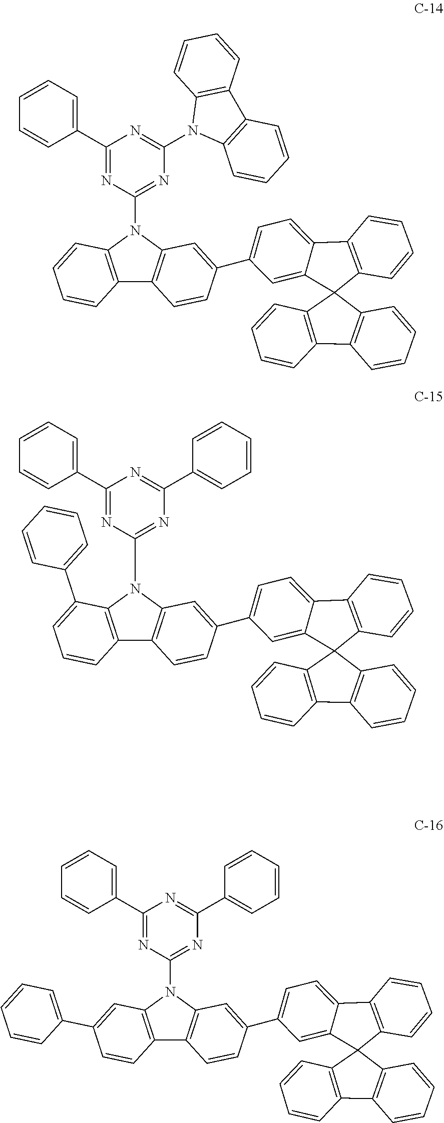 [SMILES: c1ccc(-c2nc(-n3c4ccccc4c4ccccc43)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccccc3)nc(-n3c4cc(-c5ccc6c(c5)C5(c7ccccc7-c7ccccc75)c5ccccc5-6)ccc4c4cccc(-c5ccccc5)c43)n2)cc1 ; c1ccc(-c2ccc3c4ccc(-c5ccc6c(c5)C5(c7ccccc7-c7ccccc75)c5ccccc5-6)cc4n(-c4nc(-c5ccccc5)nc(-c5ccccc5)n4)c3c2)cc1]

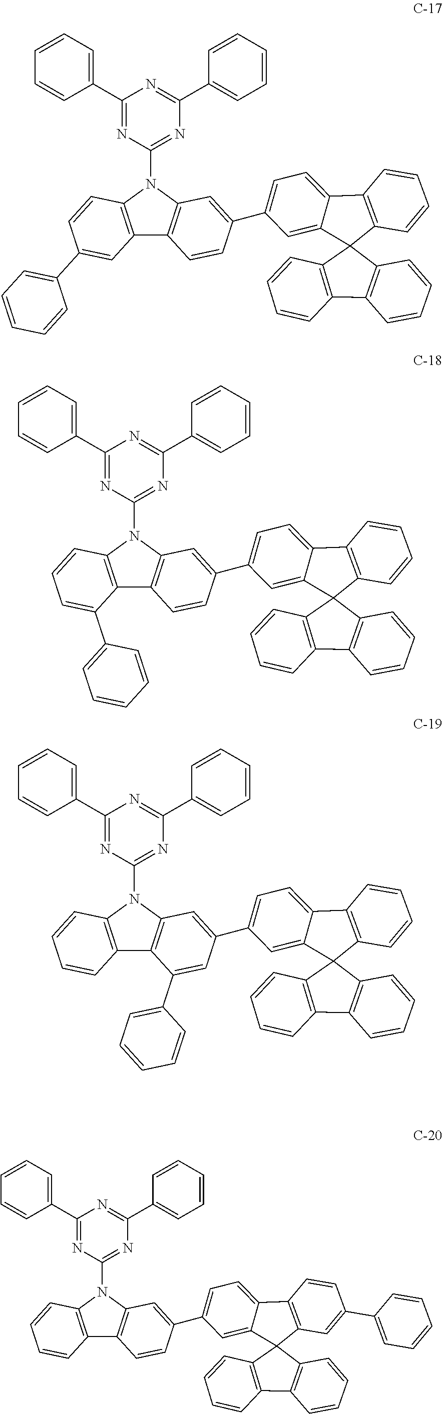 [SMILES: c1ccc(-c2ccc3c(c2)c2ccc(-c4ccc5c(c4)C4(c6ccccc6-c6ccccc64)c4ccccc4-5)cc2n3-c2nc(-c3ccccc3)nc(-c3ccccc3)n2)cc1 ; c1ccc(-c2nc(-c3ccccc3)nc(-n3c4cc(-c5ccc6c(c5)C5(c7ccccc7-c7ccccc75)c5ccccc5-6)ccc4c4c(-c5ccccc5)cccc43)n2)cc1 ; c1ccc(-c2nc(-c3ccccc3)nc(-n3c4ccccc4c4c(-c5ccccc5)cc(-c5ccc6c(c5)C5(c7ccccc7-c7ccccc75)c5ccccc5-6)cc43)n2)cc1 ; c1ccc(-c2ccc3c(c2)C2(c4ccccc4-c4ccccc42)c2cc(-c4ccc5c6ccccc6n(-c6nc(-c7ccccc7)nc(-c7ccccc7)n6)c5c4)ccc2-3)cc1]

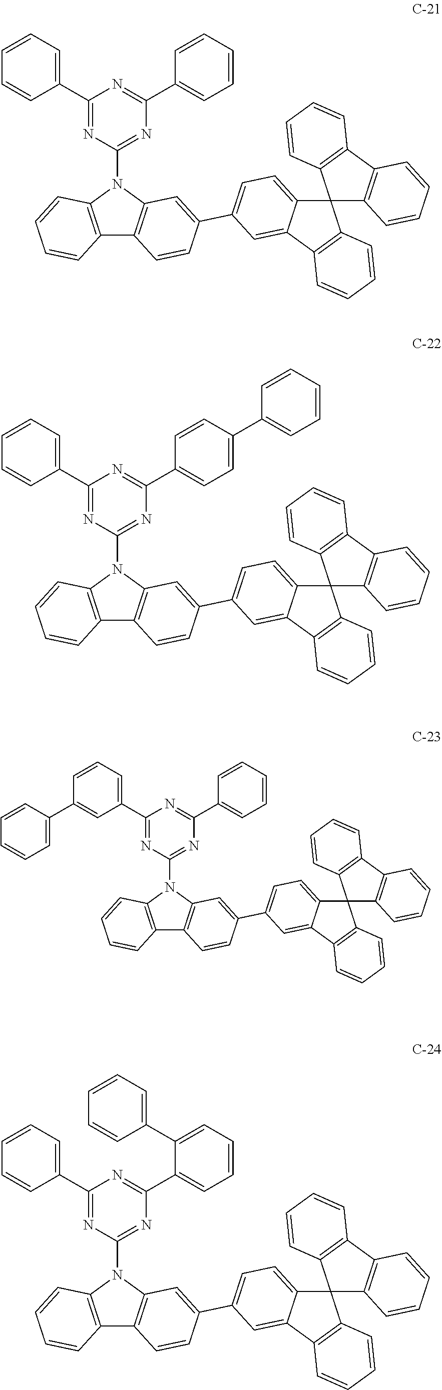 [SMILES: c1ccc(-c2nc(-c3ccccc3)nc(-n3c4ccccc4c4ccc(-c5ccc6c(c5)-c5ccccc5C65c6ccccc6-c6ccccc65)cc43)n2)cc1 ; c1ccc(-c2ccc(-c3nc(-c4ccccc4)nc(-n4c5ccccc5c5ccc(-c6ccc7c(c6)-c6ccccc6C76c7ccccc7-c7ccccc76)cc54)n3)cc2)cc1 ; c1ccc(-c2cccc(-c3nc(-c4ccccc4)nc(-n4c5ccccc5c5ccc(-c6ccc7c(c6)-c6ccccc6C76c7ccccc7-c7ccccc76)cc54)n3)c2)cc1 ; c1ccc(-c2nc(-c3ccccc3-c3ccccc3)nc(-n3c4ccccc4c4ccc(-c5ccc6c(c5)-c5ccccc5C65c6ccccc6-c6ccccc65)cc43)n2)cc1]

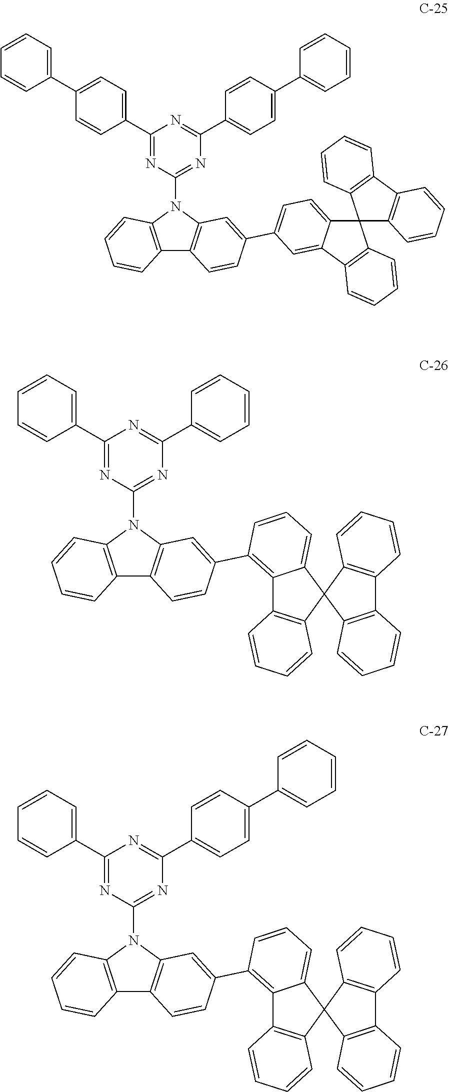 [SMILES: c1ccc(-c2ccc(-c3nc(-c4ccc(-c5ccccc5)cc4)nc(-n4c5ccccc5c5ccc(-c6ccc7c(c6)-c6ccccc6C76c7ccccc7-c7ccccc76)cc54)n3)cc2)cc1 ; c1ccc(-c2nc(-c3ccccc3)nc(-n3c4ccccc4c4ccc(-c5cccc6c5-c5ccccc5C65c6ccccc6-c6ccccc65)cc43)n2)cc1 ; c1ccc(-c2ccc(-c3nc(-c4ccccc4)nc(-n4c5ccccc5c5ccc(-c6cccc7c6-c6ccccc6C76c7ccccc7-c7ccccc76)cc54)n3)cc2)cc1]

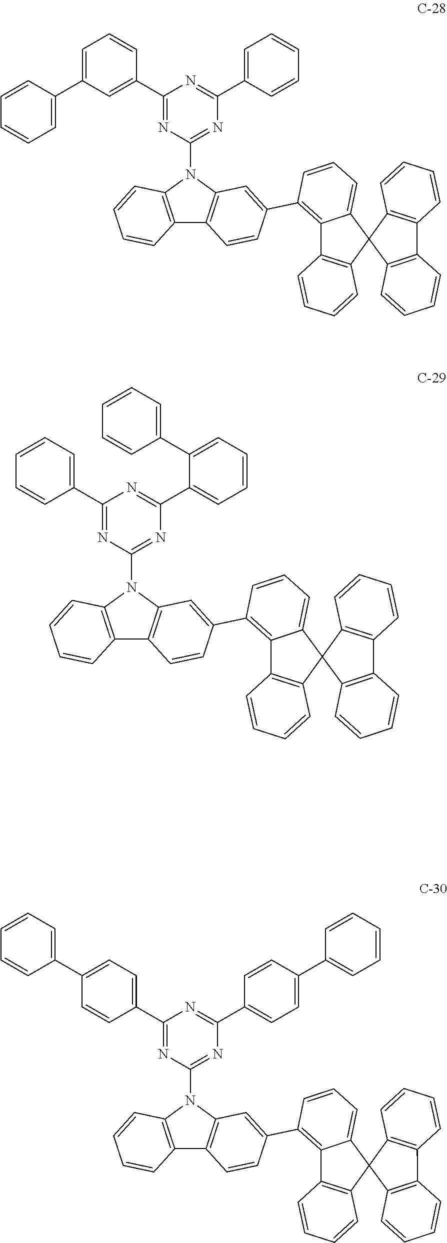 [SMILES: c1ccc(-c2cccc(-c3nc(-c4ccccc4)nc(-n4c5ccccc5c5ccc(-c6cccc7c6-c6ccccc6C76c7ccccc7-c7ccccc76)cc54)n3)c2)cc1 ; c1ccc(-c2nc(-c3ccccc3-c3ccccc3)nc(-n3c4ccccc4c4ccc(-c5cccc6c5-c5ccccc5C65c6ccccc6-c6ccccc65)cc43)n2)cc1 ; c1ccc(-c2ccc(-c3nc(-c4ccc(-c5ccccc5)cc4)nc(-n4c5ccccc5c5ccc(-c6cccc7c6-c6ccccc6C76c7ccccc7-c7ccccc76)cc54)n3)cc2)cc1]

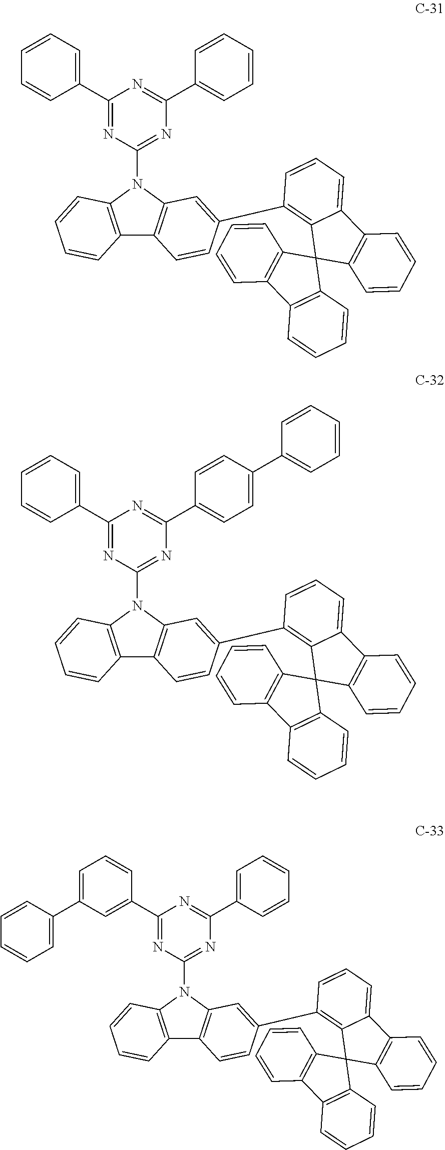 [SMILES: c1ccc(-c2nc(-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6cccc7c6C6(c8ccccc8-c8ccccc86)c6ccccc6-7)cc54)n3)cc2)cc1 ; c1ccc(-c2cccc(-c3nc(-c4ccccc4)nc(-n4c5ccccc5c5ccc(-c6cccc7c6C6(c8ccccc8-c8ccccc86)c6ccccc6-7)cc54)n3)c2)cc1]

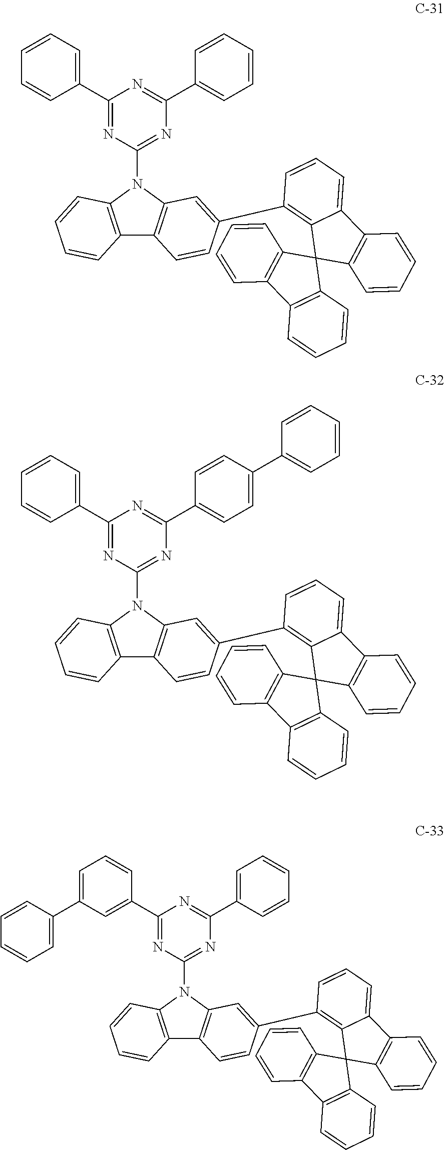 [SMILES: c1ccc(-c2nc(-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6cccc7c6C6(c8ccccc8-c8ccccc86)c6ccccc6-7)cc54)n3)cc2)cc1 ; c1ccc(-c2cccc(-c3nc(-c4ccccc4)nc(-n4c5ccccc5c5ccc(-c6cccc7c6C6(c8ccccc8-c8ccccc86)c6ccccc6-7)cc54)n3)c2)cc1]

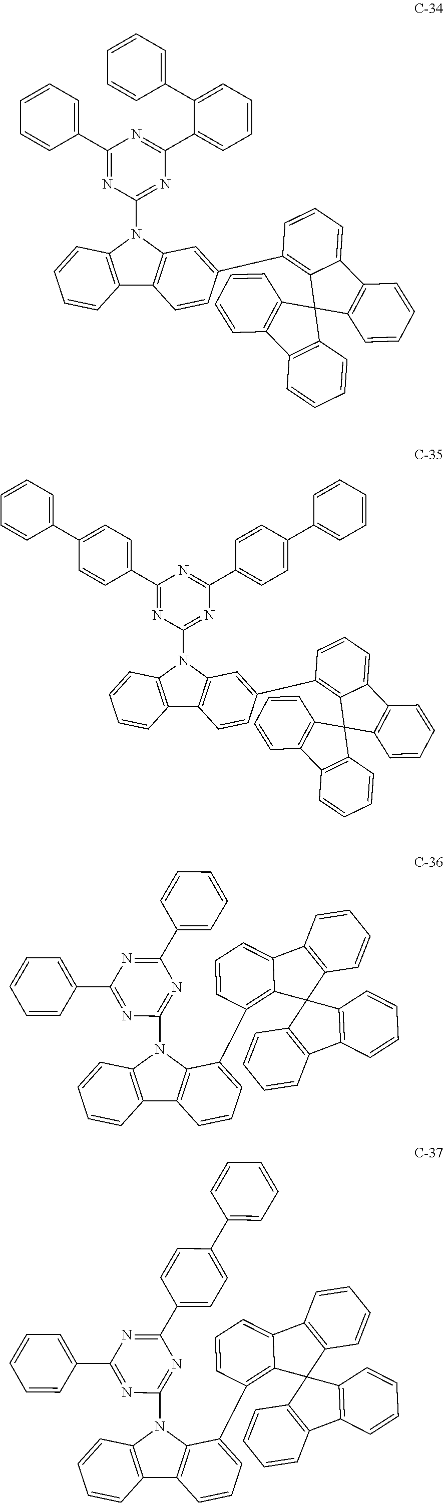

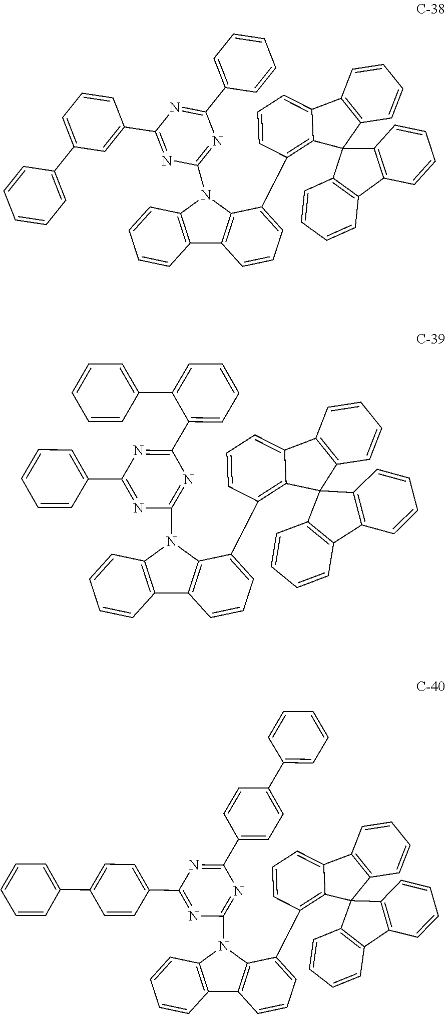 [SMILES: c1ccc(-c2cccc(-c3nc(-c4ccccc4)nc(-n4c5ccccc5c5cccc(-c6cccc7c6C6(c8ccccc8-c8ccccc86)c6ccccc6-7)c54)n3)c2)cc1 ; c1ccc(-c2nc(-c3ccccc3-c3ccccc3)nc(-n3c4ccccc4c4cccc(-c5cccc6c5C5(c7ccccc7-c7ccccc75)c5ccccc5-6)c43)n2)cc1 ; c1ccc(-c2ccc(-c3nc(-c4ccc(-c5ccccc5)cc4)nc(-n4c5ccccc5c5cccc(-c6cccc7c6C6(c8ccccc8-c8ccccc86)c6ccccc6-7)c54)n3)cc2)cc1]

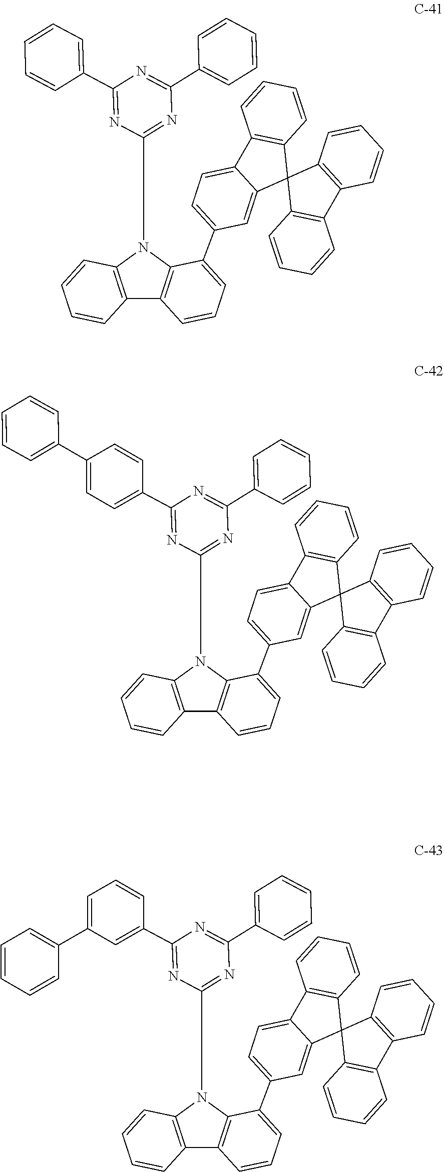 [SMILES: c1ccc(-c2nc(-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccccc4)nc(-n4c5ccccc5c5cccc(-c6ccc7c(c6)C6(c8ccccc8-c8ccccc86)c6ccccc6-7)c54)n3)cc2)cc1 ; c1ccc(-c2cccc(-c3nc(-c4ccccc4)nc(-n4c5ccccc5c5cccc(-c6ccc7c(c6)C6(c8ccccc8-c8ccccc86)c6ccccc6-7)c54)n3)c2)cc1]

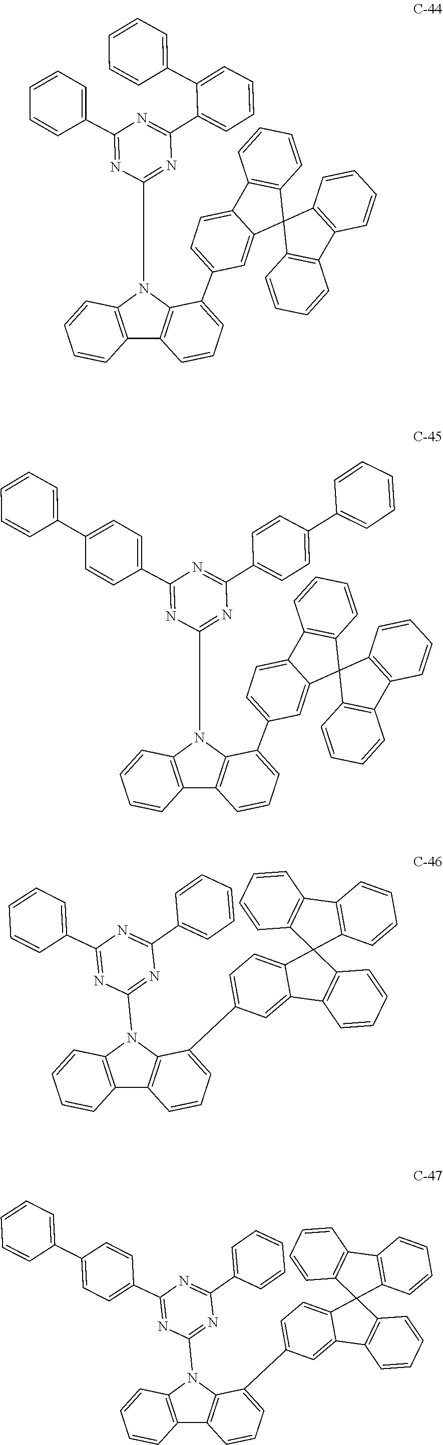 [SMILES: c1ccc(-c2nc(-c3ccccc3-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccc(-c5ccccc5)cc4)nc(-n4c5ccccc5c5cccc(-c6ccc7c(c6)C6(c8ccccc8-c8ccccc86)c6ccccc6-7)c54)n3)cc2)cc1 ; c1ccc(-c2nc(-c3ccccc3)nc(-n3c4ccccc4c4cccc(-c5ccc6c(c5)-c5ccccc5C65c6ccccc6-c6ccccc65)c43)n2)cc1 ; c1ccc(-c2ccc(-c3nc(-c4ccccc4)nc(-n4c5ccccc5c5cccc(-c6ccc7c(c6)-c6ccccc6C76c7ccccc7-c7ccccc76)c54)n3)cc2)cc1]

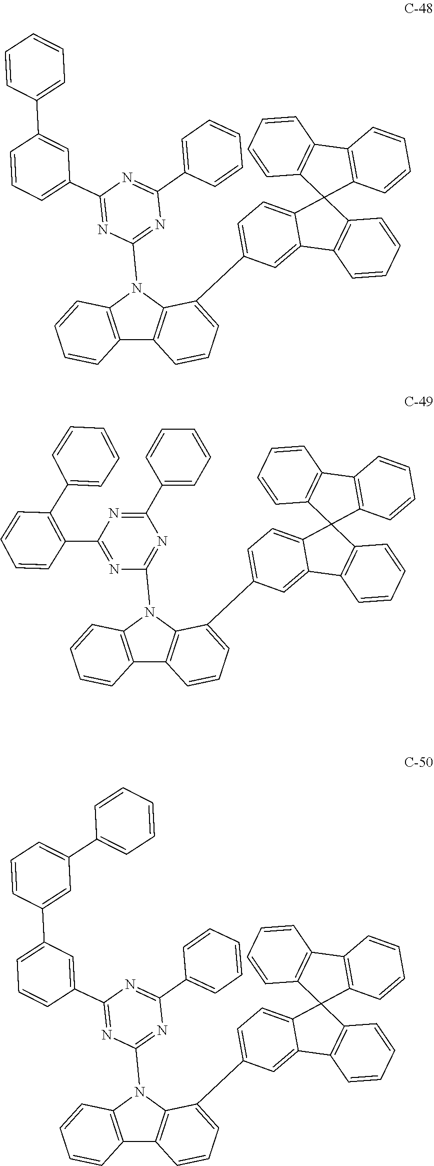 [SMILES: c1ccc(-c2cccc(-c3nc(-c4ccccc4)nc(-n4c5ccccc5c5cccc(-c6ccc7c(c6)-c6ccccc6C76c7ccccc7-c7ccccc76)c54)n3)c2)cc1 ; c1ccc(-c2nc(-c3ccccc3-c3ccccc3)nc(-n3c4ccccc4c4cccc(-c5ccc6c(c5)-c5ccccc5C65c6ccccc6-c6ccccc65)c43)n2)cc1 ; c1ccc(-c2cccc(-c3cccc(-c4nc(-c5ccccc5)nc(-n5c6ccccc6c6cccc(-c7ccc8c(c7)-c7ccccc7C87c8ccccc8-c8ccccc87)c65)n4)c3)c2)cc1]

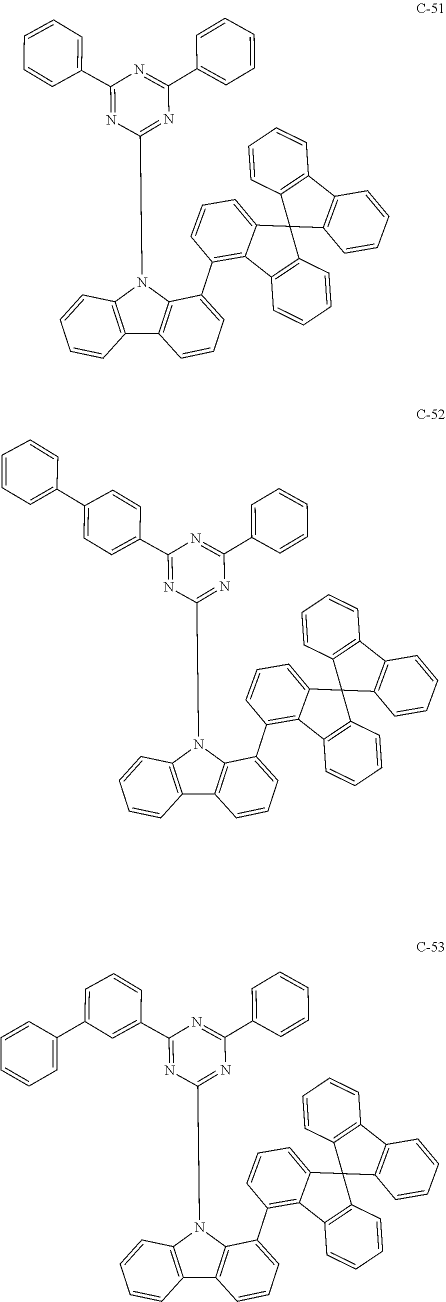 [SMILES: c1ccc(-c2nc(-c3ccccc3)nc(-n3c4ccccc4c4cccc(-c5cccc6c5-c5ccccc5C65c6ccccc6-c6ccccc65)c43)n2)cc1 ; c1ccc(-c2ccc(-c3nc(-c4ccccc4)nc(-n4c5ccccc5c5cccc(-c6cccc7c6-c6ccccc6C76c7ccccc7-c7ccccc76)c54)n3)cc2)cc1 ; c1ccc(-c2cccc(-c3nc(-c4ccccc4)nc(-n4c5ccccc5c5cccc(-c6cccc7c6-c6ccccc6C76c7ccccc7-c7ccccc76)c54)n3)c2)cc1]

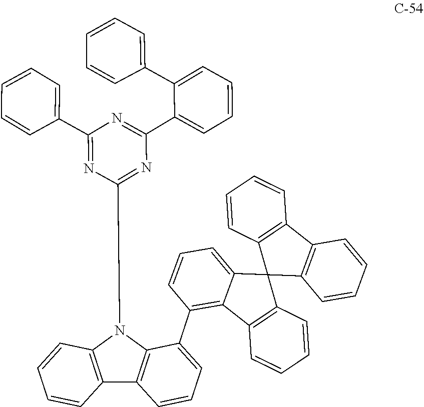 [SMILES: c1ccc(-c2nc(-c3ccccc3-c3ccccc3)nc(-n3c4ccccc4c4cccc(-c5cccc6c5-c5ccccc5C65c6ccccc6-c6ccccc65)c43)n2)cc1]

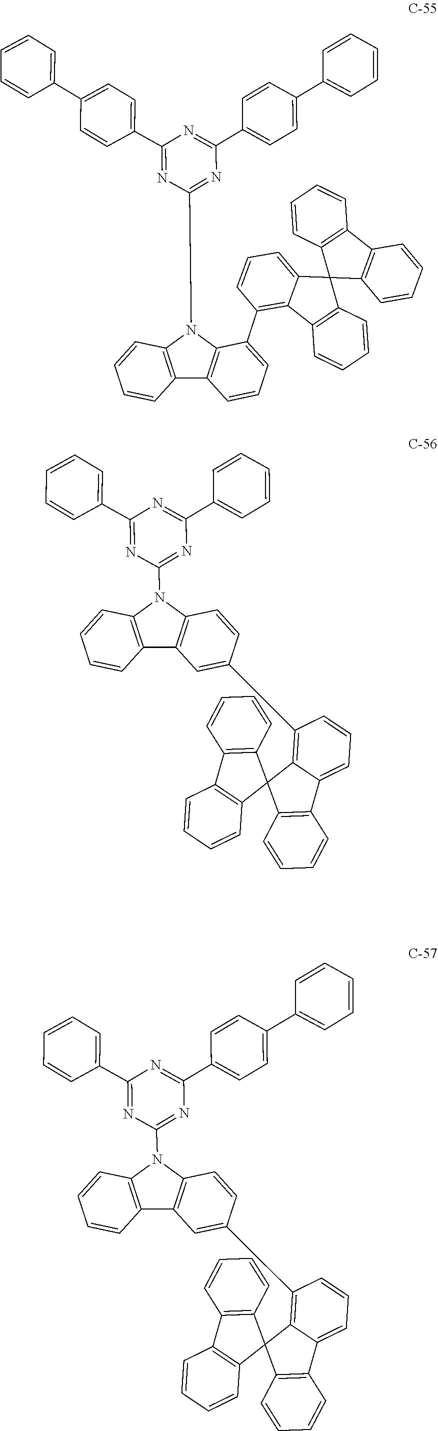 [SMILES: c1ccc(-c2ccc(-c3nc(-c4ccc(-c5ccccc5)cc4)nc(-n4c5ccccc5c5cccc(-c6cccc7c6-c6ccccc6C76c7ccccc7-c7ccccc76)c54)n3)cc2)cc1 ; c1ccc(-c2nc(-c3ccccc3)nc(-n3c4ccccc4c4cc(-c5cccc6c5C5(c7ccccc7-c7ccccc75)c5ccccc5-6)ccc43)n2)cc1 ; c1ccc(-c2ccc(-c3nc(-c4ccccc4)nc(-n4c5ccccc5c5cc(-c6cccc7c6C6(c8ccccc8-c8ccccc86)c6ccccc6-7)ccc54)n3)cc2)cc1]

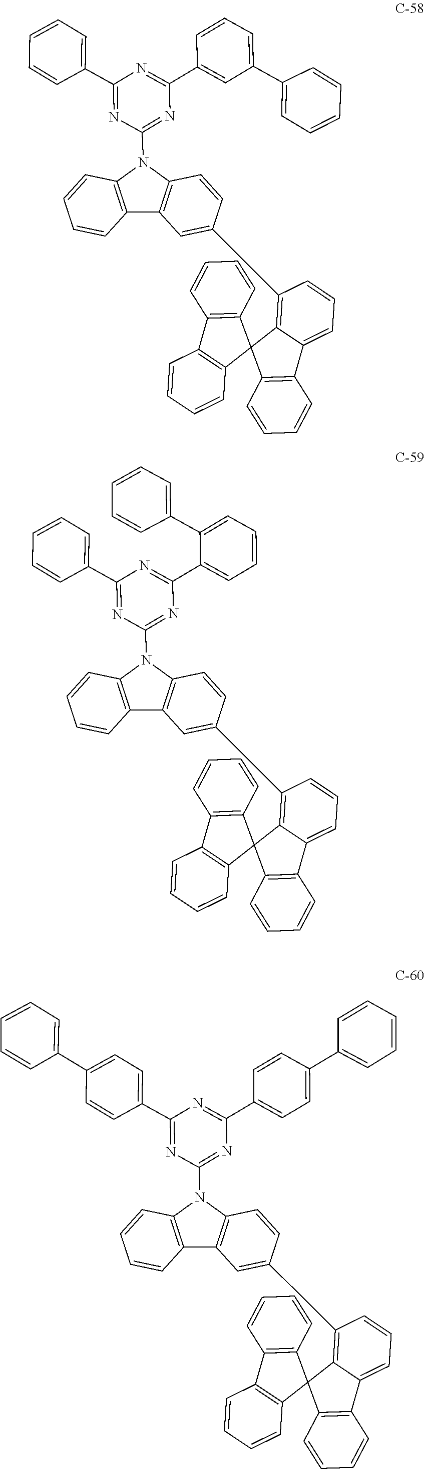 [SMILES: c1ccc(-c2cccc(-c3nc(-c4ccccc4)nc(-n4c5ccccc5c5cc(-c6cccc7c6C6(c8ccccc8-c8ccccc86)c6ccccc6-7)ccc54)n3)c2)cc1 ; c1ccc(-c2nc(-c3ccccc3-c3ccccc3)nc(-n3c4ccccc4c4cc(-c5cccc6c5C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6cccc7c6C6(c8ccccc8-c8ccccc86)c6ccccc6-7)ccc54)n3)cc2)cc1]

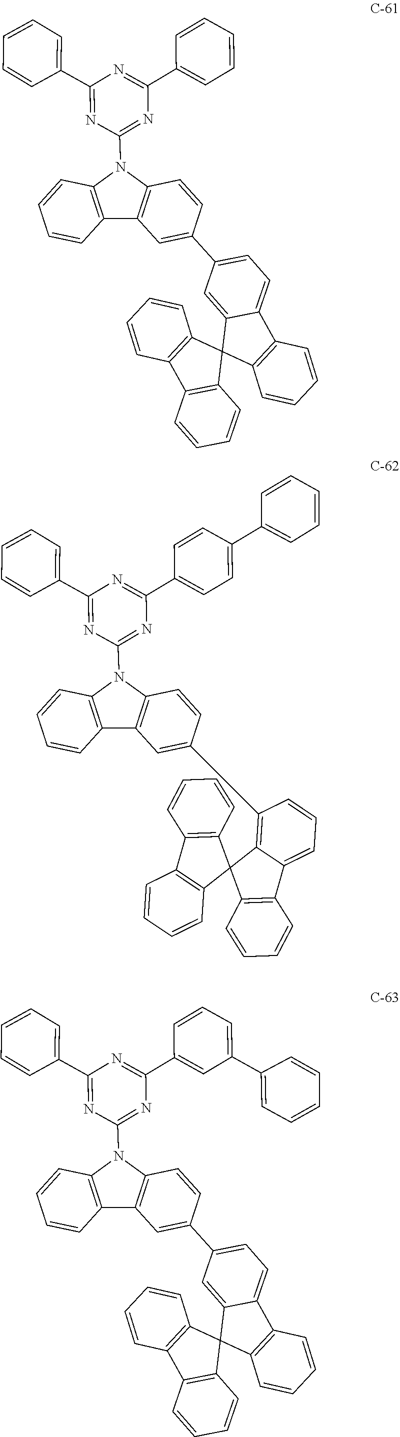 [SMILES: c1ccc(-c2nc(-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccccc4)nc(-n4c5ccccc5c5cc(-c6cccc7c6C6(c8ccccc8-c8ccccc86)c6ccccc6-7)ccc54)n3)cc2)cc1 ; c1ccc(-c2cccc(-c3nc(-c4ccccc4)nc(-n4c5ccccc5c5cc(-c6ccc7c(c6)C6(c8ccccc8-c8ccccc86)c6ccccc6-7)ccc54)n3)c2)cc1]

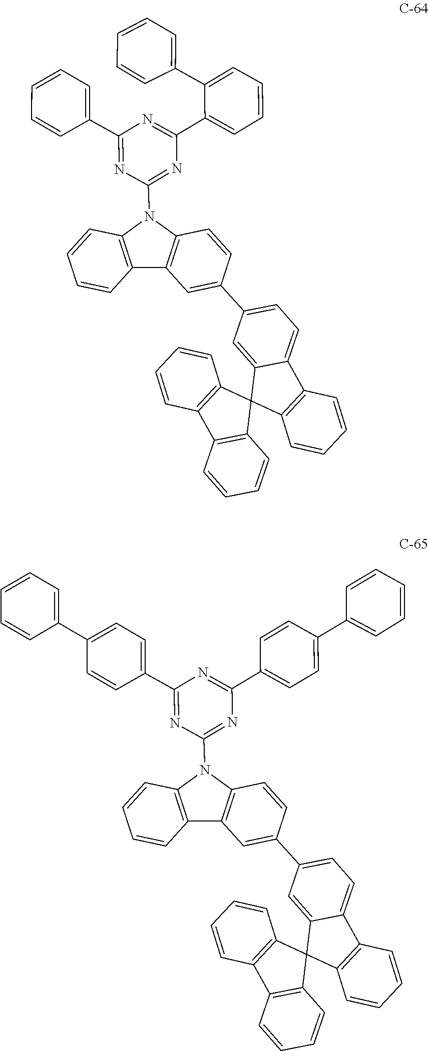 [SMILES: c1ccc(-c2nc(-c3ccccc3-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6ccc7c(c6)C6(c8ccccc8-c8ccccc86)c6ccccc6-7)ccc54)n3)cc2)cc1]

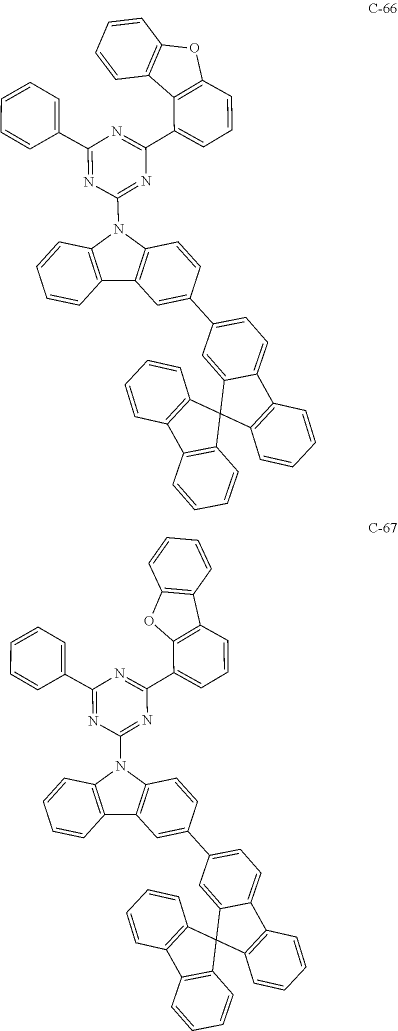

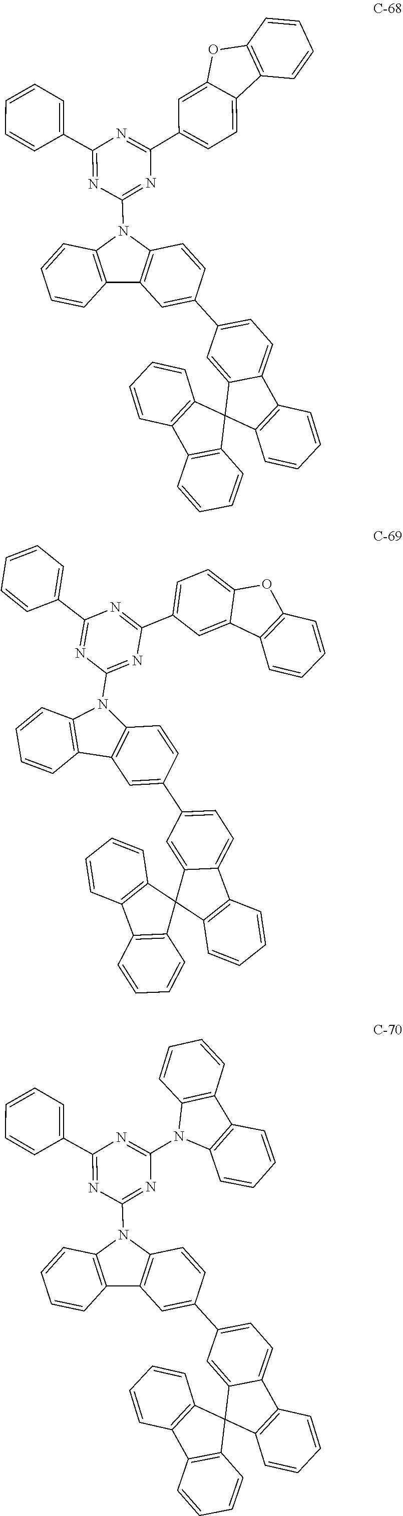 [SMILES: c1ccc(-c2nc(-c3ccc4c(c3)oc3ccccc34)nc(-n3c4ccccc4c4cc(-c5ccc6c(c5)C5(c7ccccc7-c7ccccc75)c5ccccc5-6)ccc43)n2)cc1 ; c1ccc(-c2nc(-c3ccc4oc5ccccc5c4c3)nc(-n3c4ccccc4c4cc(-c5ccc6c(c5)C5(c7ccccc7-c7ccccc75)c5ccccc5-6)ccc43)n2)cc1 ; c1ccc(-c2nc(-n3c4ccccc4c4ccccc43)nc(-n3c4ccccc4c4cc(-c5ccc6c(c5)C5(c7ccccc7-c7ccccc75)c5ccccc5-6)ccc43)n2)cc1]

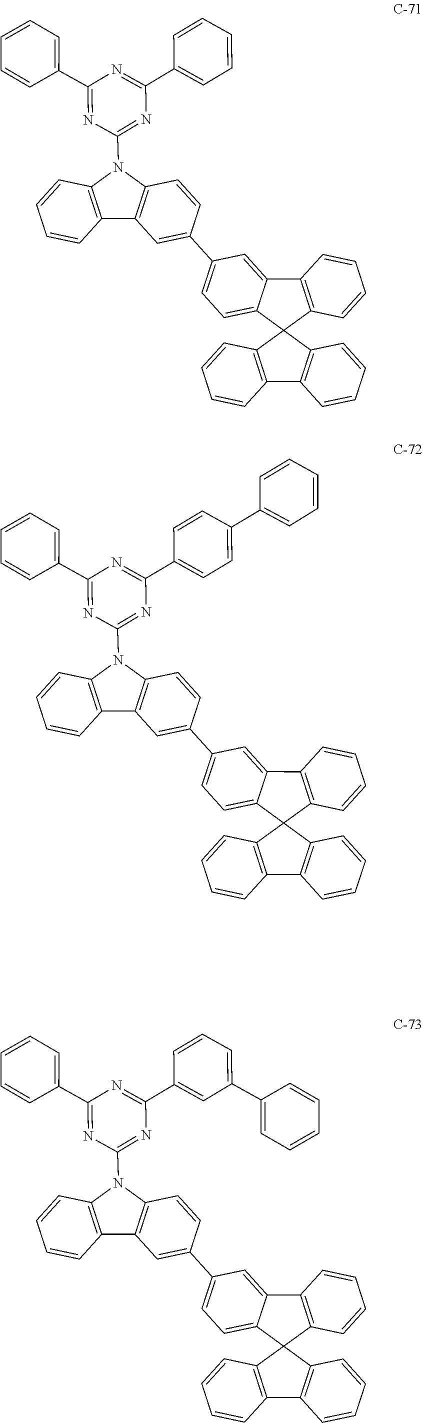 [SMILES: c1ccc(-c2nc(-c3ccccc3)nc(-n3c4ccccc4c4cc(-c5ccc6c(c5)-c5ccccc5C65c6ccccc6-c6ccccc65)ccc43)n2)cc1 ; c1ccc(-c2ccc(-c3nc(-c4ccccc4)nc(-n4c5ccccc5c5cc(-c6ccc7c(c6)-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6ccc7c(c6)-c6ccccc6C76c7ccccc7-c7ccccc76)ccc54)n3)c2)cc1]

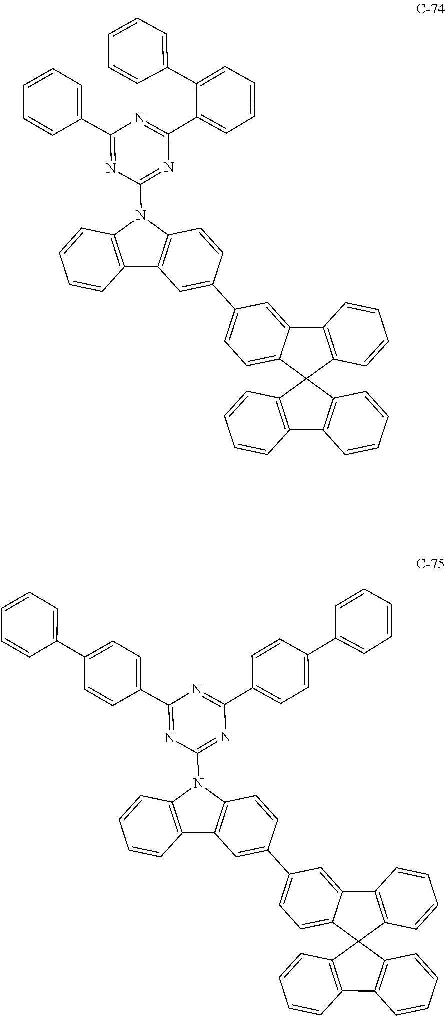 [SMILES: c1ccc(-c2nc(-c3ccccc3-c3ccccc3)nc(-n3c4ccccc4c4cc(-c5ccc6c(c5)-c5ccccc5C65c6ccccc6-c6ccccc65)ccc43)n2)cc1 ; c1ccc(-c2ccc(-c3nc(-c4ccc(-c5ccccc5)cc4)nc(-n4c5ccccc5c5cc(-c6ccc7c(c6)-c6ccccc6C76c7ccccc7-c7ccccc76)ccc54)n3)cc2)cc1]

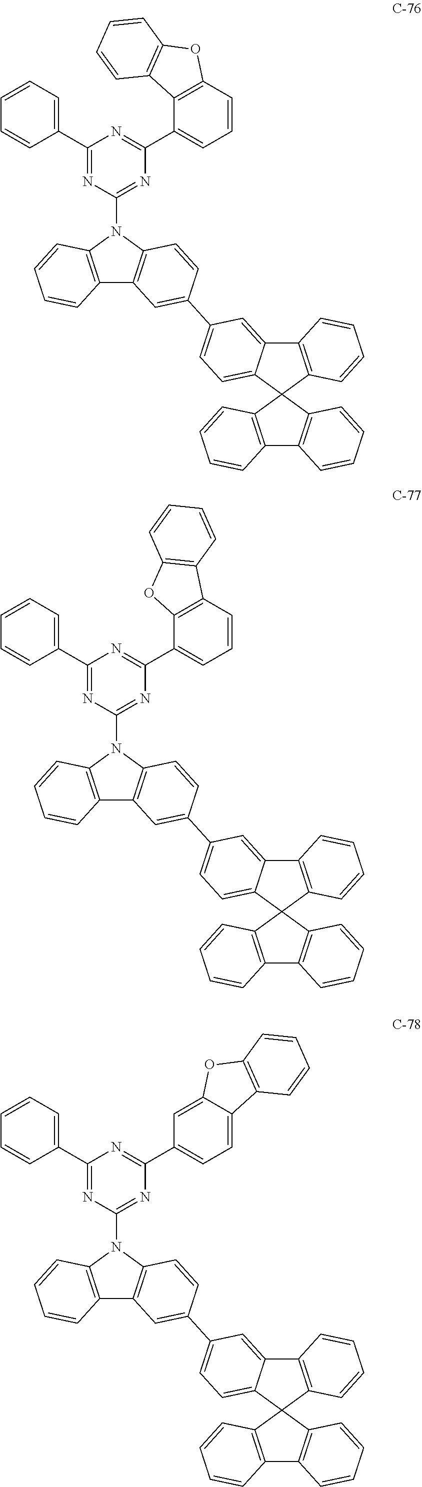

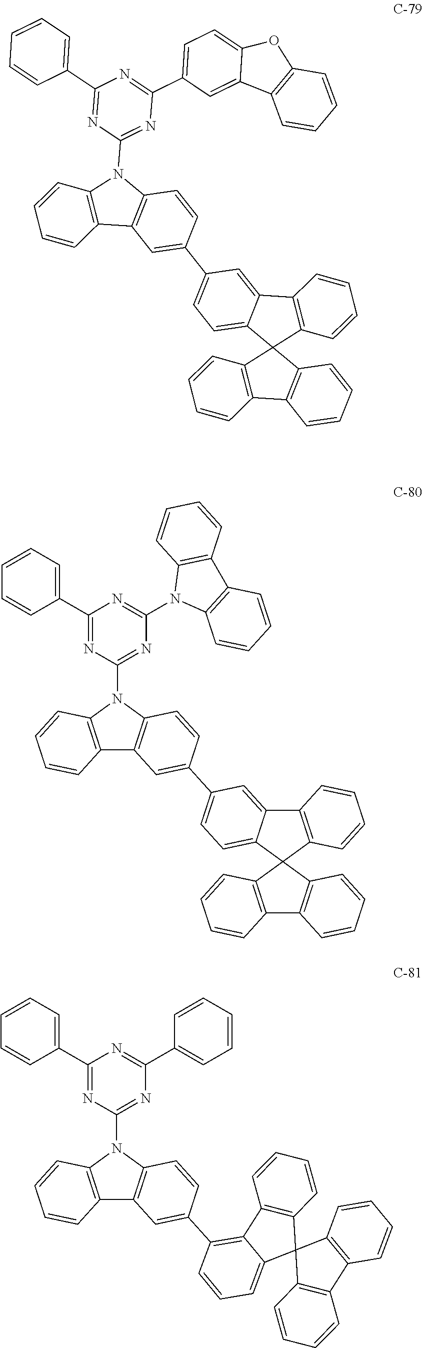 [SMILES: c1ccc(-c2nc(-c3ccc4oc5ccccc5c4c3)nc(-n3c4ccccc4c4cc(-c5ccc6c(c5)-c5ccccc5C65c6ccccc6-c6ccccc65)ccc43)n2)cc1 ; c1ccc(-c2nc(-n3c4ccccc4c4ccccc43)nc(-n3c4ccccc4c4cc(-c5ccc6c(c5)-c5ccccc5C65c6ccccc6-c6ccccc65)ccc43)n2)cc1 ; c1ccc(-c2nc(-c3ccccc3)nc(-n3c4ccccc4c4cc(-c5cccc6c5-c5ccccc5C65c6ccccc6-c6ccccc65)ccc43)n2)cc1]

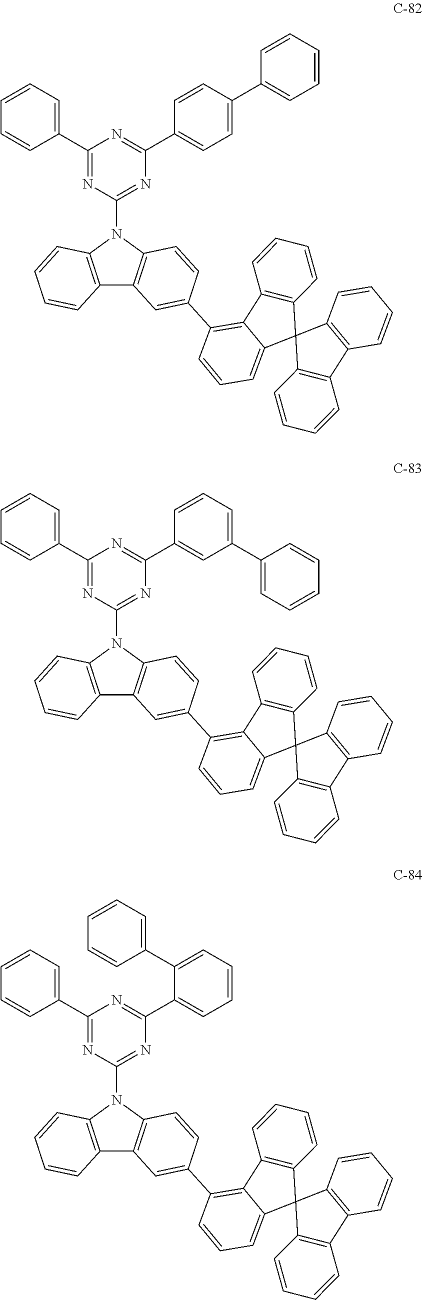 [SMILES: c1ccc(-c2ccc(-c3nc(-c4ccccc4)nc(-n4c5ccccc5c5cc(-c6cccc7c6-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6cccc7c6-c6ccccc6C76c7ccccc7-c7ccccc76)ccc54)n3)c2)cc1 ; c1ccc(-c2nc(-c3ccccc3-c3ccccc3)nc(-n3c4ccccc4c4cc(-c5cccc6c5-c5ccccc5C65c6ccccc6-c6ccccc65)ccc43)n2)cc1]

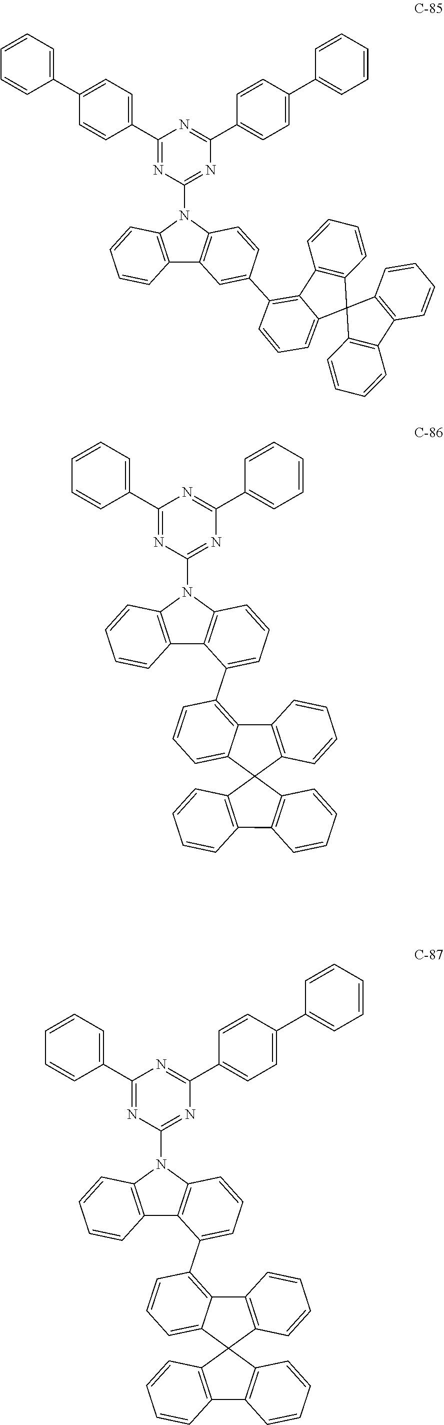 [SMILES: c1ccc(-c2ccc(-c3nc(-c4ccc(-c5ccccc5)cc4)nc(-n4c5ccccc5c5cc(-c6cccc7c6-c6ccccc6C76c7ccccc7-c7ccccc76)ccc54)n3)cc2)cc1 ; c1ccc(-c2nc(-c3ccccc3)nc(-n3c4ccccc4c4c(-c5cccc6c5-c5ccccc5C65c6ccccc6-c6ccccc65)cccc43)n2)cc1 ; c1ccc(-c2ccc(-c3nc(-c4ccccc4)nc(-n4c5ccccc5c5c(-c6cccc7c6-c6ccccc6C76c7ccccc7-c7ccccc76)cccc54)n3)cc2)cc1]

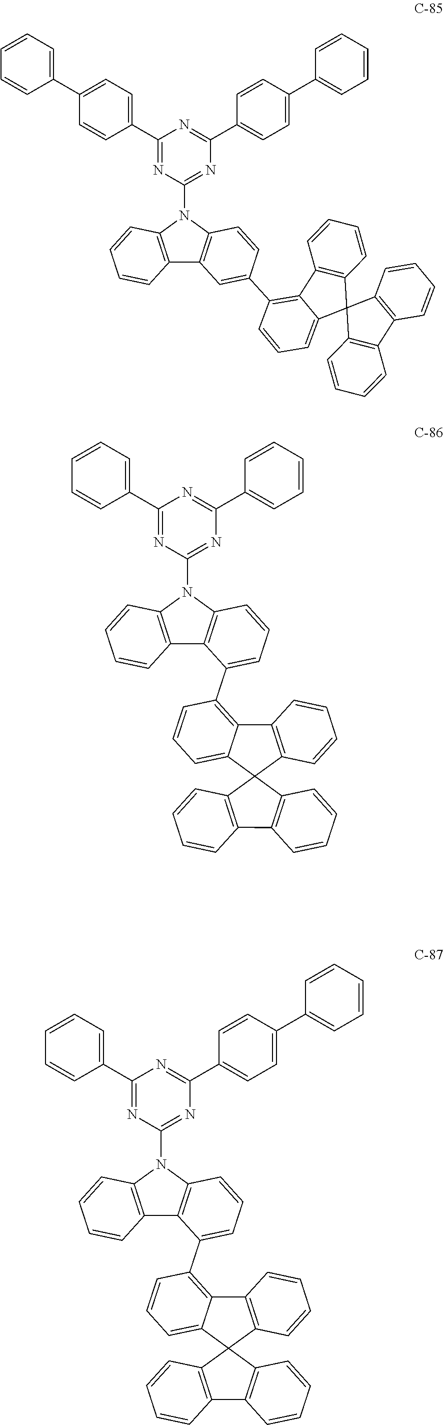 [SMILES: c1ccc(-c2ccc(-c3nc(-c4ccc(-c5ccccc5)cc4)nc(-n4c5ccccc5c5cc(-c6cccc7c6-c6ccccc6C76c7ccccc7-c7ccccc76)ccc54)n3)cc2)cc1 ; c1ccc(-c2nc(-c3ccccc3)nc(-n3c4ccccc4c4c(-c5cccc6c5-c5ccccc5C65c6ccccc6-c6ccccc65)cccc43)n2)cc1 ; c1ccc(-c2ccc(-c3nc(-c4ccccc4)nc(-n4c5ccccc5c5c(-c6cccc7c6-c6ccccc6C76c7ccccc7-c7ccccc76)cccc54)n3)cc2)cc1]

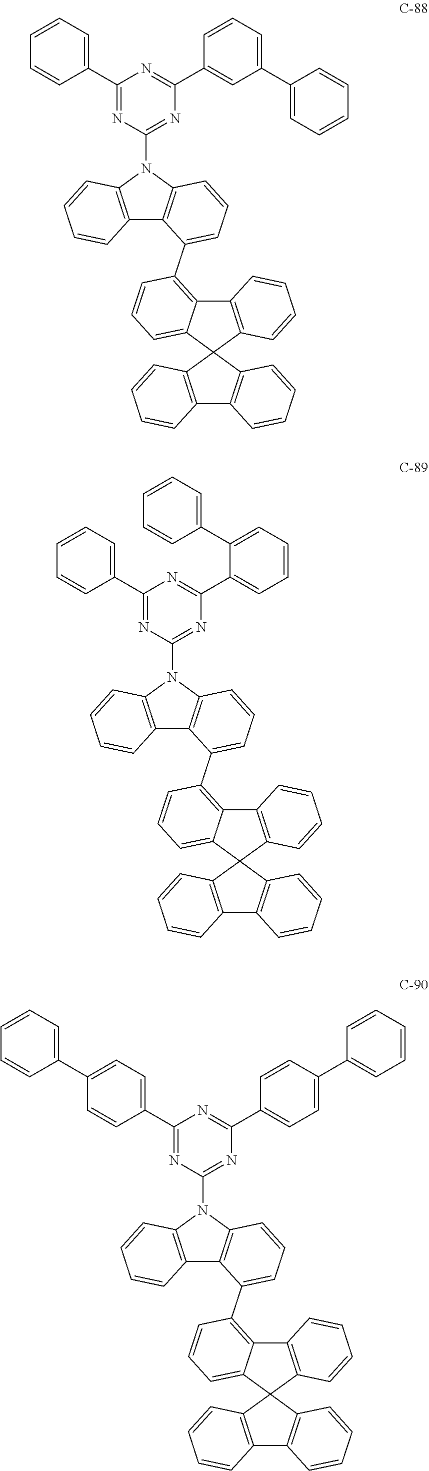 [SMILES: c1ccc(-c2cccc(-c3nc(-c4ccccc4)nc(-n4c5ccccc5c5c(-c6cccc7c6-c6ccccc6C76c7ccccc7-c7ccccc76)cccc54)n3)c2)cc1 ; c1ccc(-c2nc(-c3ccccc3-c3ccccc3)nc(-n3c4ccccc4c4c(-c5cccc6c5-c5ccccc5C65c6ccccc6-c6ccccc65)cccc43)n2)cc1 ; c1ccc(-c2ccc(-c3nc(-c4ccc(-c5ccccc5)cc4)nc(-n4c5ccccc5c5c(-c6cccc7c6-c6ccccc6C76c7ccccc7-c7ccccc76)cccc54)n3)cc2)cc1]

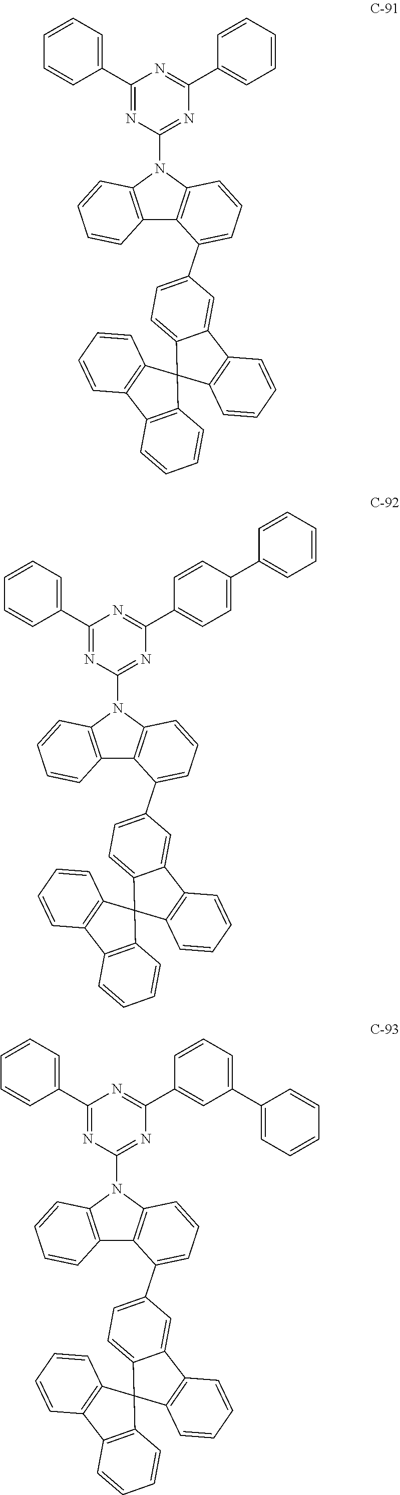 [SMILES: c1ccc(-c2nc(-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccccc4)nc(-n4c5ccccc5c5c(-c6ccc7c(c6)-c6ccccc6C76c7ccccc7-c7ccccc76)cccc54)n3)cc2)cc1 ; c1ccc(-c2cccc(-c3nc(-c4ccccc4)nc(-n4c5ccccc5c5c(-c6ccc7c(c6)-c6ccccc6C76c7ccccc7-c7ccccc76)cccc54)n3)c2)cc1]

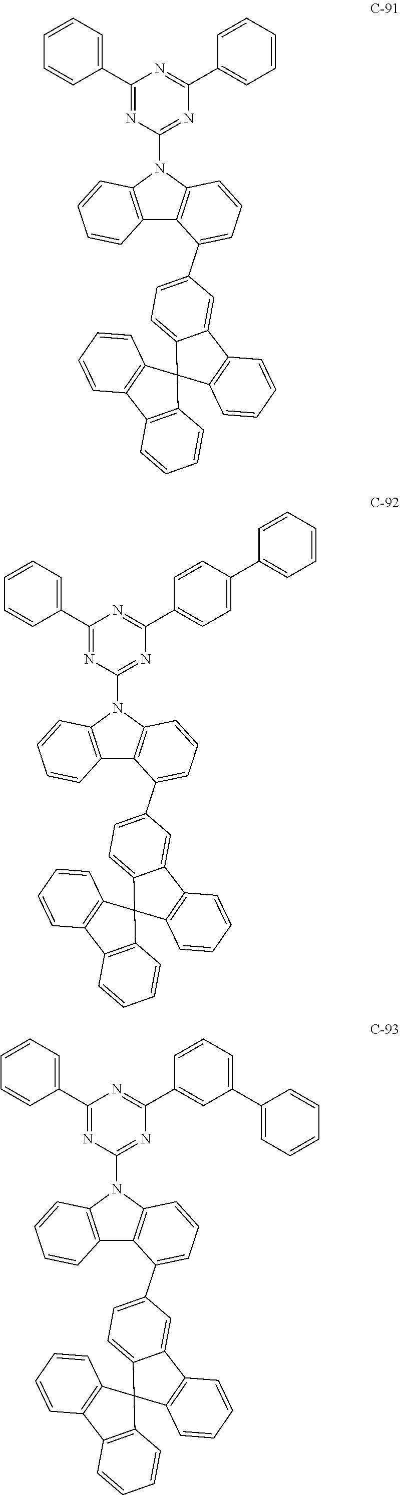 [SMILES: c1ccc(-c2nc(-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccccc4)nc(-n4c5ccccc5c5c(-c6ccc7c(c6)-c6ccccc6C76c7ccccc7-c7ccccc76)cccc54)n3)cc2)cc1 ; c1ccc(-c2cccc(-c3nc(-c4ccccc4)nc(-n4c5ccccc5c5c(-c6ccc7c(c6)-c6ccccc6C76c7ccccc7-c7ccccc76)cccc54)n3)c2)cc1]

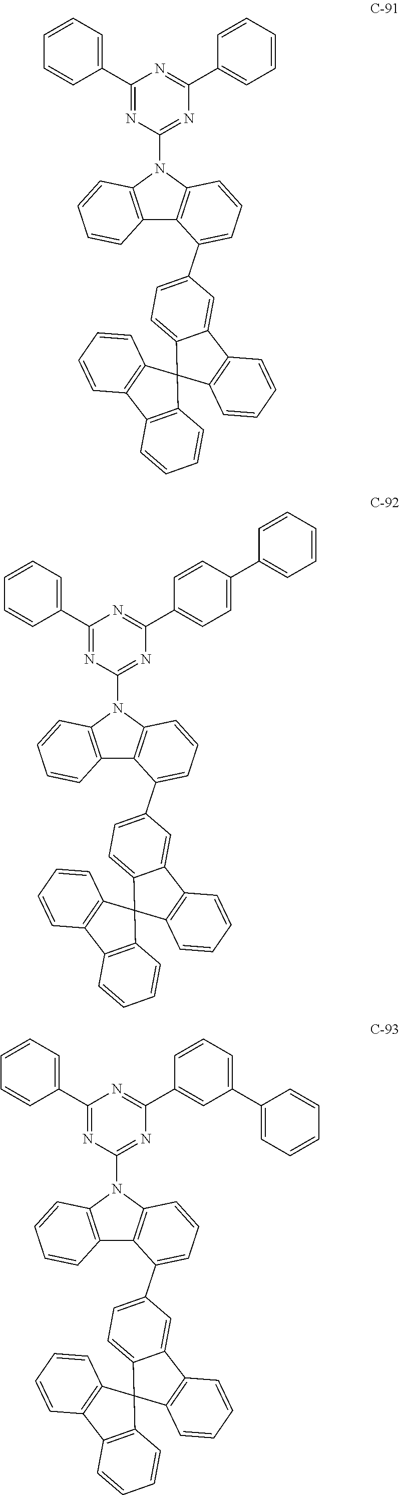 [SMILES: c1ccc(-c2nc(-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccccc4)nc(-n4c5ccccc5c5c(-c6ccc7c(c6)-c6ccccc6C76c7ccccc7-c7ccccc76)cccc54)n3)cc2)cc1 ; c1ccc(-c2cccc(-c3nc(-c4ccccc4)nc(-n4c5ccccc5c5c(-c6ccc7c(c6)-c6ccccc6C76c7ccccc7-c7ccccc76)cccc54)n3)c2)cc1]

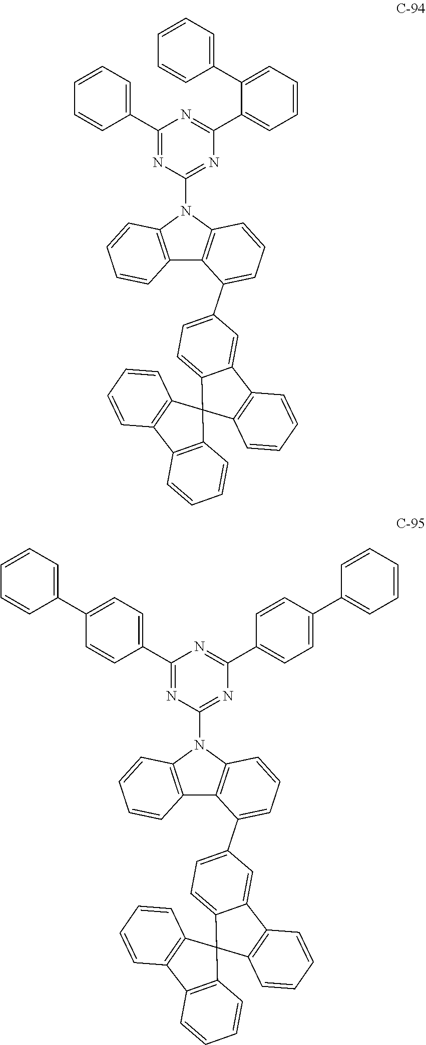 [SMILES: c1ccc(-c2nc(-c3ccccc3-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccc(-c5ccccc5)cc4)nc(-n4c5ccccc5c5c(-c6ccc7c(c6)-c6ccccc6C76c7ccccc7-c7ccccc76)cccc54)n3)cc2)cc1]

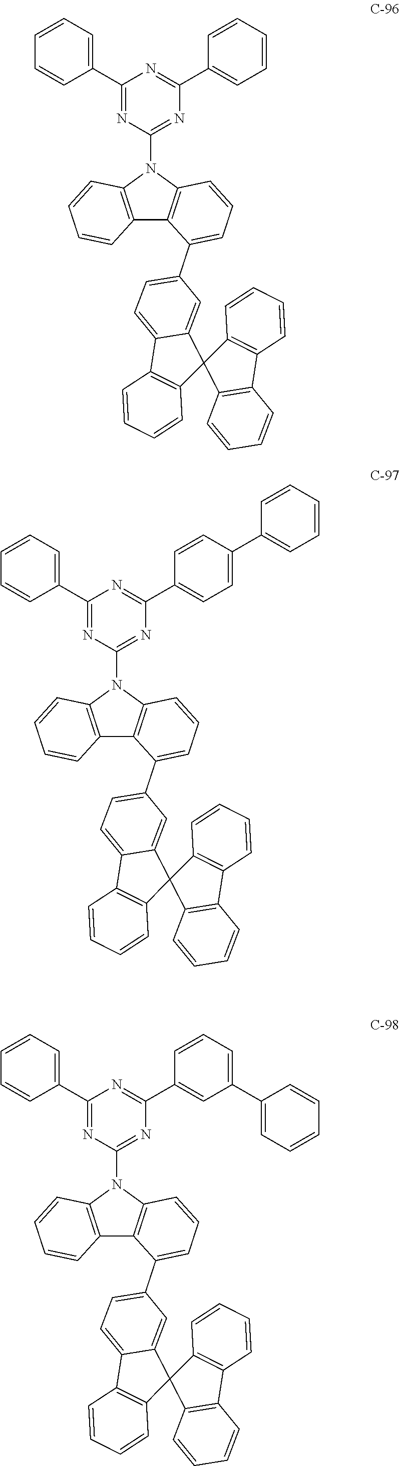 [SMILES: c1ccc(-c2nc(-c3ccccc3)nc(-n3c4ccccc4c4c(-c5ccc6c(c5)C5(c7ccccc7-c7ccccc75)c5ccccc5-6)cccc43)n2)cc1 ; c1ccc(-c2ccc(-c3nc(-c4ccccc4)nc(-n4c5ccccc5c5c(-c6ccc7c(c6)C6(c8ccccc8-c8ccccc86)c6ccccc6-7)cccc54)n3)cc2)cc1 ; c1ccc(-c2cccc(-c3nc(-c4ccccc4)nc(-n4c5ccccc5c5c(-c6ccc7c(c6)C6(c8ccccc8-c8ccccc86)c6ccccc6-7)cccc54)n3)c2)cc1]

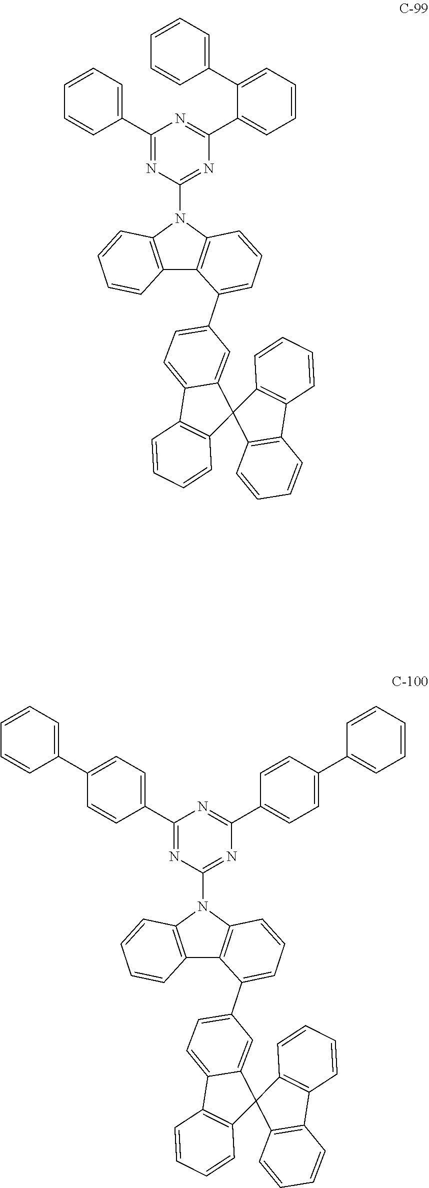 [SMILES: c1ccc(-c2nc(-c3ccccc3-c3ccccc3)nc(-n3c4ccccc4c4c(-c5ccc6c(c5)C5(c7ccccc7-c7ccccc75)c5ccccc5-6)cccc43)n2)cc1 ; c1ccc(-c2ccc(-c3nc(-c4ccc(-c5ccccc5)cc4)nc(-n4c5ccccc5c5c(-c6ccc7c(c6)C6(c8ccccc8-c8ccccc86)c6ccccc6-7)cccc54)n3)cc2)cc1]

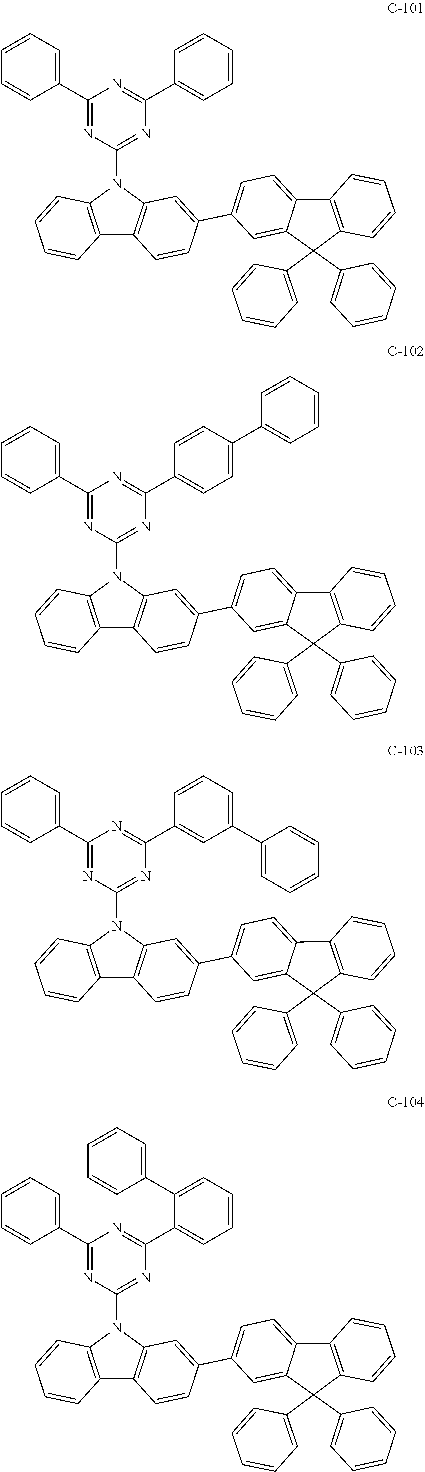 [SMILES: c1ccc(-c2nc(-c3ccccc3)nc(-n3c4ccccc4c4ccc(-c5ccc6c(c5)C(c5ccccc5)(c5ccccc5)c5ccccc5-6)cc43)n2)cc1 ; c1ccc(-c2ccc(-c3nc(-c4ccccc4)nc(-n4c5ccccc5c5ccc(-c6ccc7c(c6)C(c6ccccc6)(c6ccccc6)c6ccccc6-7)cc54)n3)cc2)cc1 ; c1ccc(-c2cccc(-c3nc(-c4ccccc4)nc(-n4c5ccccc5c5ccc(-c6ccc7c(c6)C(c6ccccc6)(c6ccccc6)c6ccccc6-7)cc54)n3)c2)cc1 ; c1ccc(-c2nc(-c3ccccc3-c3ccccc3)nc(-n3c4ccccc4c4ccc(-c5ccc6c(c5)C(c5ccccc5)(c5ccccc5)c5ccccc5-6)cc43)n2)cc1]

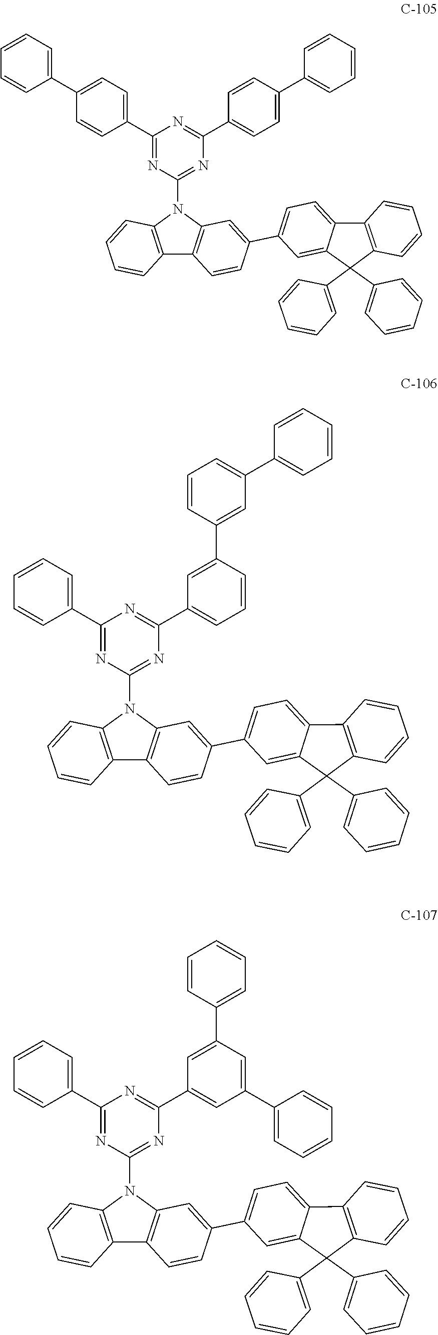 [SMILES: c1ccc(-c2ccc(-c3nc(-c4ccc(-c5ccccc5)cc4)nc(-n4c5ccccc5c5ccc(-c6ccc7c(c6)C(c6ccccc6)(c6ccccc6)c6ccccc6-7)cc54)n3)cc2)cc1 ; c1ccc(-c2cccc(-c3cccc(-c4nc(-c5ccccc5)nc(-n5c6ccccc6c6ccc(-c7ccc8c(c7)C(c7ccccc7)(c7ccccc7)c7ccccc7-8)cc65)n4)c3)c2)cc1 ; c1ccc(-c2cc(-c3ccccc3)cc(-c3nc(-c4ccccc4)nc(-n4c5ccccc5c5ccc(-c6ccc7c(c6)C(c6ccccc6)(c6ccccc6)c6ccccc6-7)cc54)n3)c2)cc1]

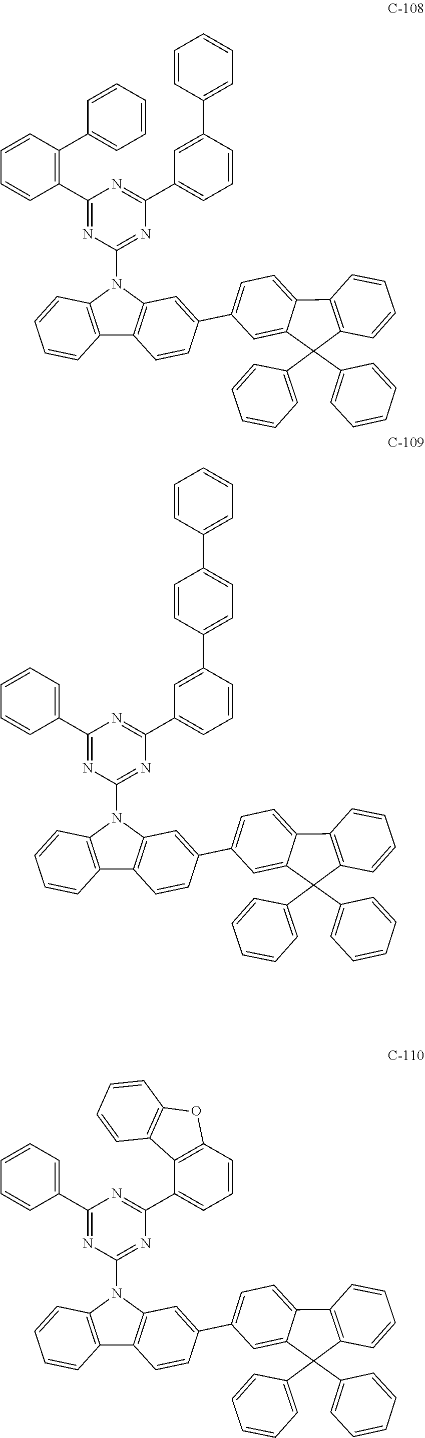 [SMILES: c1ccc(-c2cccc(-c3nc(-c4ccccc4-c4ccccc4)nc(-n4c5ccccc5c5ccc(-c6ccc7c(c6)C(c6ccccc6)(c6ccccc6)c6ccccc6-7)cc54)n3)c2)cc1 ; c1ccc(-c2ccc(-c3cccc(-c4nc(-c5ccccc5)nc(-n5c6ccccc6c6ccc(-c7ccc8c(c7)C(c7ccccc7)(c7ccccc7)c7ccccc7-8)cc65)n4)c3)cc2)cc1 ; c1ccc(-c2nc(-c3cccc4oc5ccccc5c34)nc(-n3c4ccccc4c4ccc(-c5ccc6c(c5)C(c5ccccc5)(c5ccccc5)c5ccccc5-6)cc43)n2)cc1]

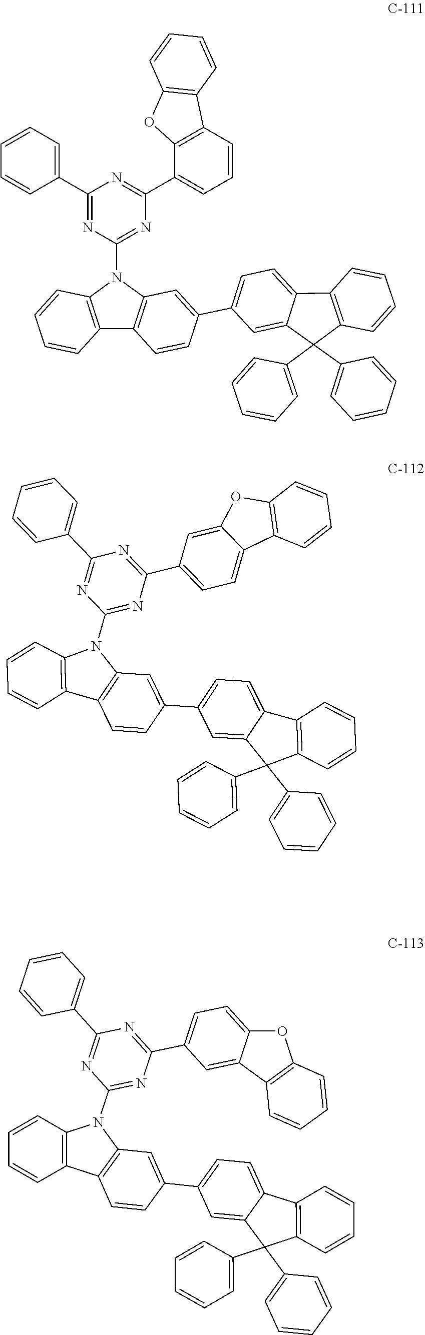 [SMILES: c1ccc(-c2nc(-c3cccc4c3oc3ccccc34)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccc4c(c3)oc3ccccc34)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccc4oc5ccccc5c4c3)nc(-n3c4ccccc4c4ccc(-c5ccc6c(c5)C(c5ccccc5)(c5ccccc5)c5ccccc5-6)cc43)n2)cc1]

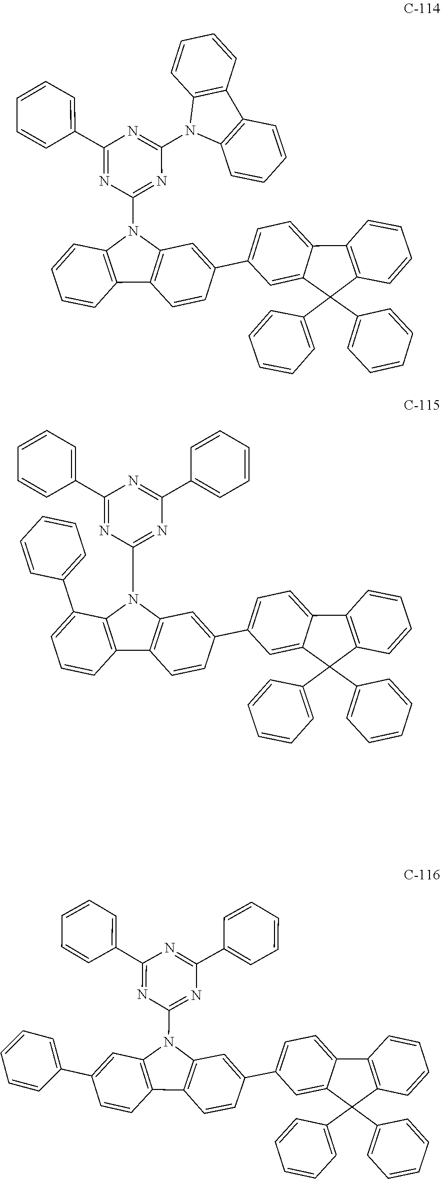 [SMILES: c1ccc(-c2nc(-n3c4ccccc4c4ccccc43)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccccc3)nc(-n3c4cc(-c5ccc6c(c5)C(c5ccccc5)(c5ccccc5)c5ccccc5-6)ccc4c4cccc(-c5ccccc5)c43)n2)cc1 ; c1ccc(-c2ccc3c4ccc(-c5ccc6c(c5)C(c5ccccc5)(c5ccccc5)c5ccccc5-6)cc4n(-c4nc(-c5ccccc5)nc(-c5ccccc5)n4)c3c2)cc1]

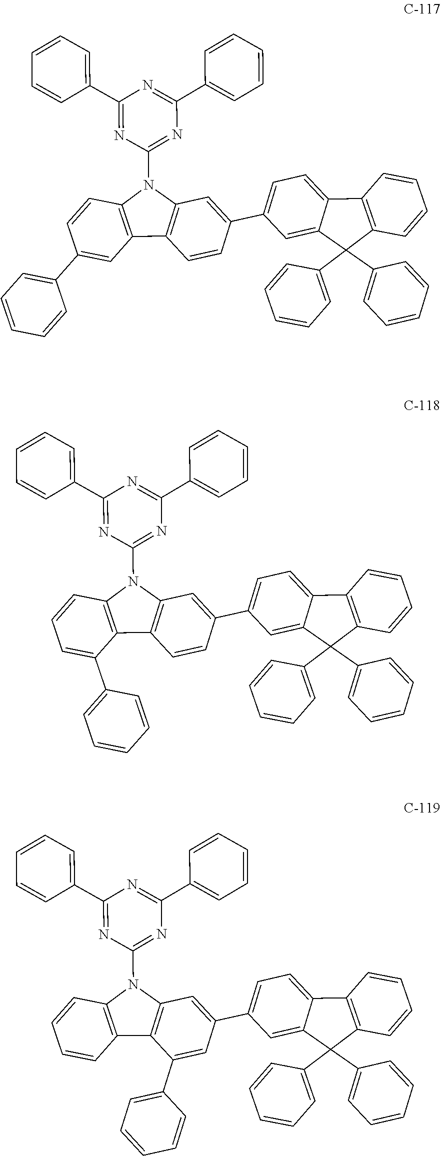 [SMILES: c1ccc(-c2ccc3c(c2)c2ccc(-c4ccc5c(c4)C(c4ccccc4)(c4ccccc4)c4ccccc4-5)cc2n3-c2nc(-c3ccccc3)nc(-c3ccccc3)n2)cc1 ; c1ccc(-c2nc(-c3ccccc3)nc(-n3c4cc(-c5ccc6c(c5)C(c5ccccc5)(c5ccccc5)c5ccccc5-6)ccc4c4c(-c5ccccc5)cccc43)n2)cc1 ; c1ccc(-c2nc(-c3ccccc3)nc(-n3c4ccccc4c4c(-c5ccccc5)cc(-c5ccc6c(c5)C(c5ccccc5)(c5ccccc5)c5ccccc5-6)cc43)n2)cc1]

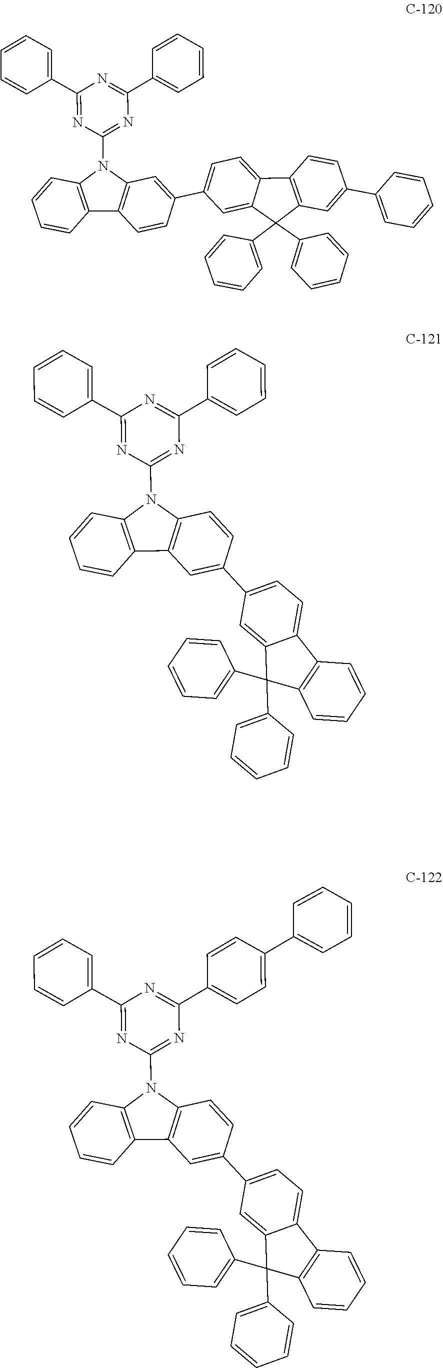 [SMILES: c1ccc(-c2ccc3c(c2)C(c2ccccc2)(c2ccccc2)c2cc(-c4ccc5c6ccccc6n(-c6nc(-c7ccccc7)nc(-c7ccccc7)n6)c5c4)ccc2-3)cc1 ; c1ccc(-c2nc(-c3ccccc3)nc(-n3c4ccccc4c4cc(-c5ccc6c(c5)C(c5ccccc5)(c5ccccc5)c5ccccc5-6)ccc43)n2)cc1 ; c1ccc(-c2ccc(-c3nc(-c4ccccc4)nc(-n4c5ccccc5c5cc(-c6ccc7c(c6)C(c6ccccc6)(c6ccccc6)c6ccccc6-7)ccc54)n3)cc2)cc1]

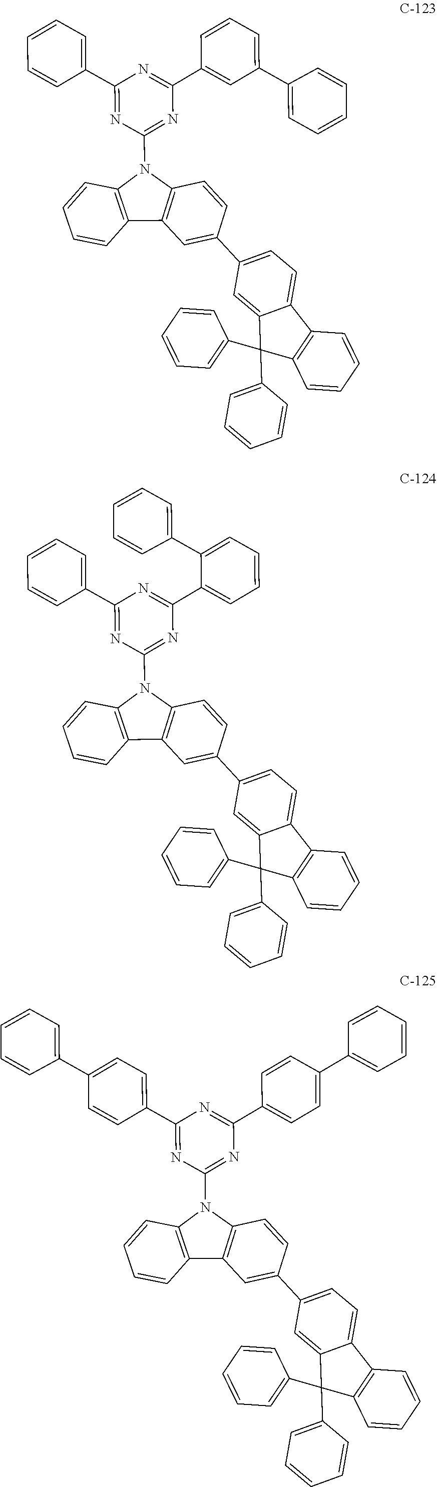 [SMILES: c1ccc(-c2cccc(-c3nc(-c4ccccc4)nc(-n4c5ccccc5c5cc(-c6ccc7c(c6)C(c6ccccc6)(c6ccccc6)c6ccccc6-7)ccc54)n3)c2)cc1 ; c1ccc(-c2nc(-c3ccccc3-c3ccccc3)nc(-n3c4ccccc4c4cc(-c5ccc6c(c5)C(c5ccccc5)(c5ccccc5)c5ccccc5-6)ccc43)n2)cc1 ; c1ccc(-c2ccc(-c3nc(-c4ccc(-c5ccccc5)cc4)nc(-n4c5ccccc5c5cc(-c6ccc7c(c6)C(c6ccccc6)(c6ccccc6)c6ccccc6-7)ccc54)n3)cc2)cc1]

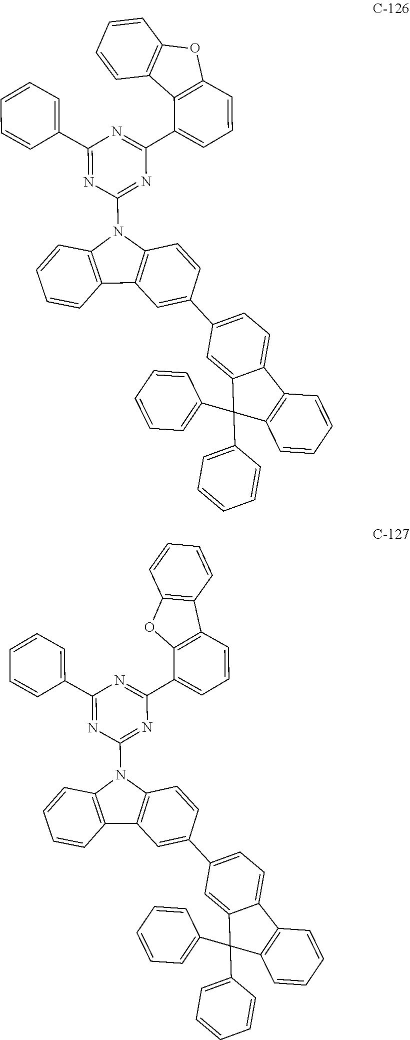

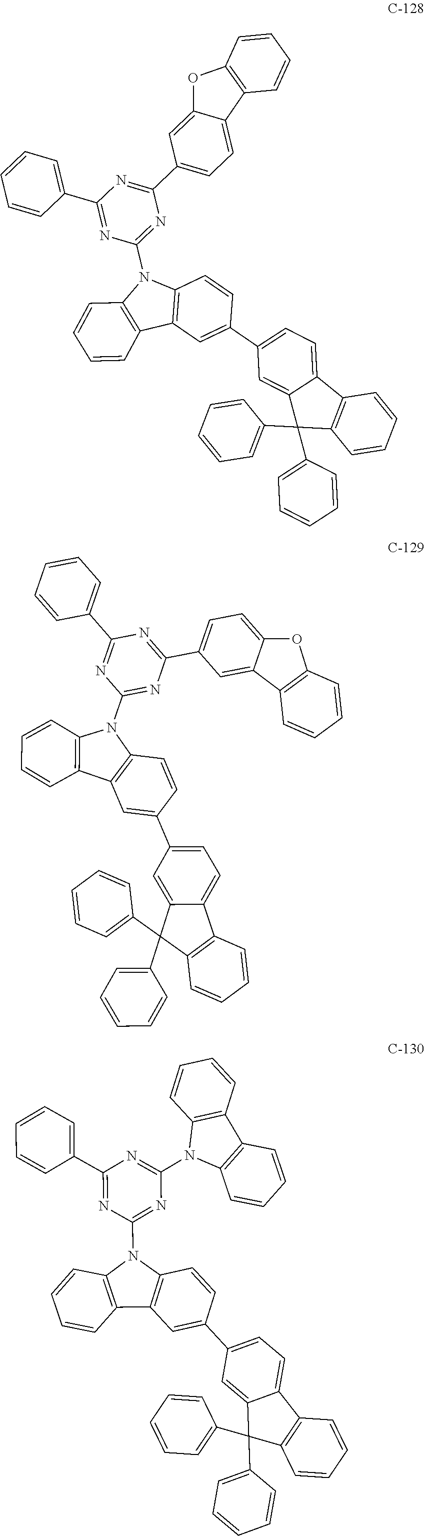 [SMILES: c1ccc(-c2nc(-c3ccc4c(c3)oc3ccccc34)nc(-n3c4ccccc4c4cc(-c5ccc6c(c5)C(c5ccccc5)(c5ccccc5)c5ccccc5-6)ccc43)n2)cc1 ; c1ccc(-c2nc(-c3ccc4oc5ccccc5c4c3)nc(-n3c4ccccc4c4cc(-c5ccc6c(c5)C(c5ccccc5)(c5ccccc5)c5ccccc5-6)ccc43)n2)cc1 ; c1ccc(-c2nc(-n3c4ccccc4c4ccccc43)nc(-n3c4ccccc4c4cc(-c5ccc6c(c5)C(c5ccccc5)(c5ccccc5)c5ccccc5-6)ccc43)n2)cc1]

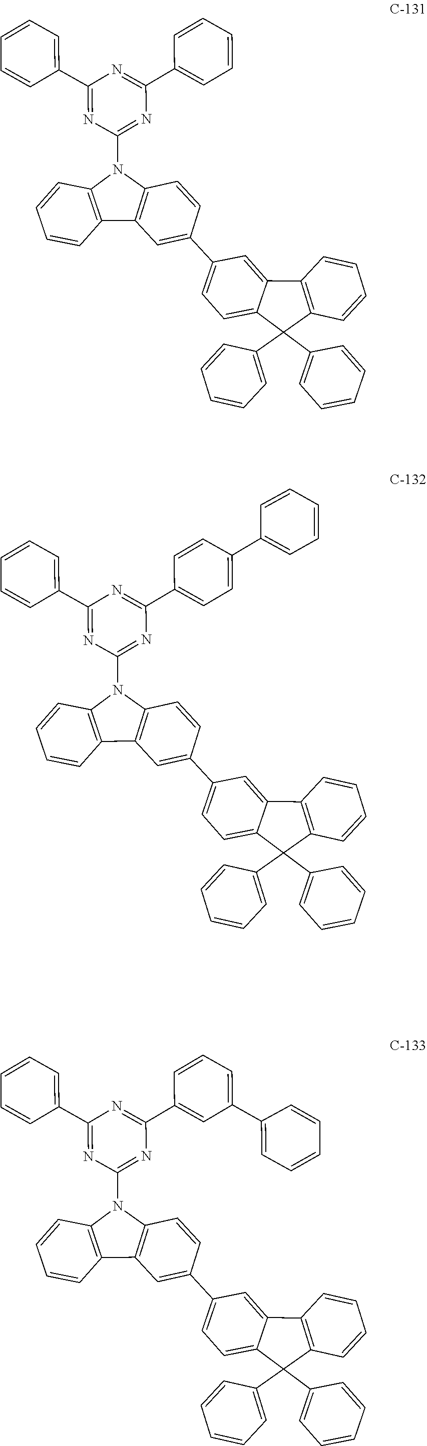 [SMILES: c1ccc(-c2nc(-c3ccccc3)nc(-n3c4ccccc4c4cc(-c5ccc6c(c5)-c5ccccc5C6(c5ccccc5)c5ccccc5)ccc43)n2)cc1 ; c1ccc(-c2ccc(-c3nc(-c4ccccc4)nc(-n4c5ccccc5c5cc(-c6ccc7c(c6)-c6ccccc6C7(c6ccccc6)c6ccccc6)ccc54)n3)cc2)cc1 ; c1ccc(-c2cccc(-c3nc(-c4ccccc4)nc(-n4c5ccccc5c5cc(-c6ccc7c(c6)-c6ccccc6C7(c6ccccc6)c6ccccc6)ccc54)n3)c2)cc1]

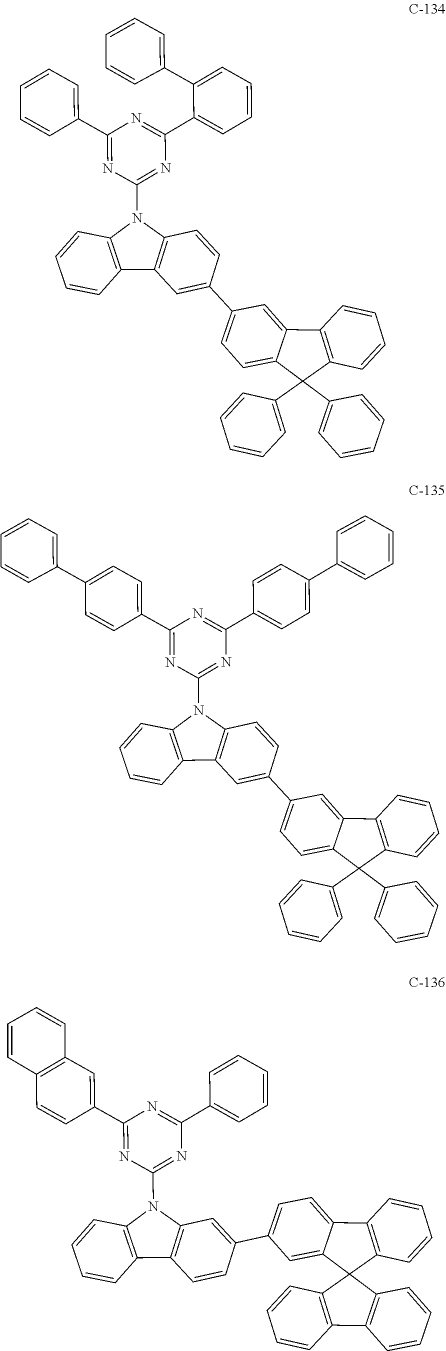 [SMILES: c1ccc(-c2nc(-c3ccccc3-c3ccccc3)nc(-n3c4ccccc4c4cc(-c5ccc6c(c5)-c5ccccc5C6(c5ccccc5)c5ccccc5)ccc43)n2)cc1 ; c1ccc(-c2ccc(-c3nc(-c4ccc(-c5ccccc5)cc4)nc(-n4c5ccccc5c5cc(-c6ccc7c(c6)-c6ccccc6C7(c6ccccc6)c6ccccc6)ccc54)n3)cc2)cc1 ; c1ccc(-c2nc(-c3ccc4ccccc4c3)nc(-n3c4ccccc4c4ccc(-c5ccc6c(c5)C5(c7ccccc7-c7ccccc75)c5ccccc5-6)cc43)n2)cc1]

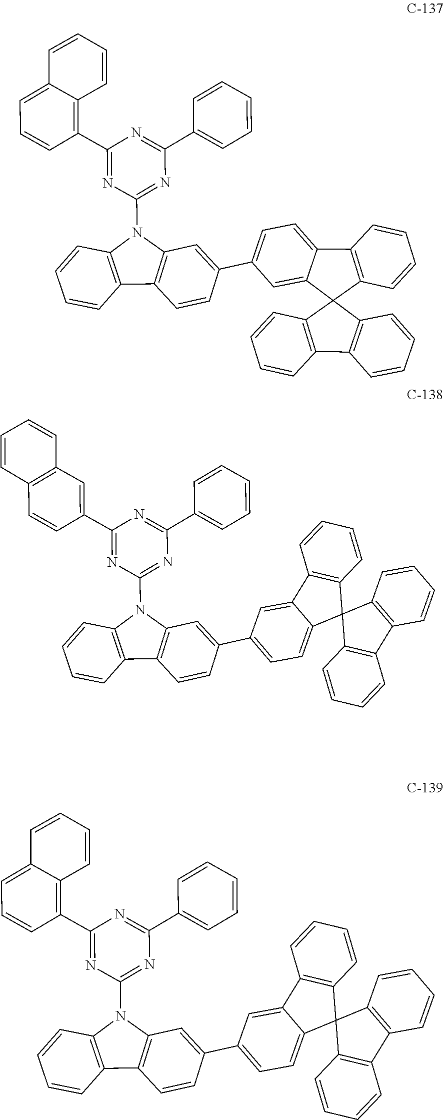 [SMILES: c1ccc(-c2nc(-c3cccc4ccccc34)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccc4ccccc4c3)nc(-n3c4ccccc4c4ccc(-c5ccc6c(c5)-c5ccccc5C65c6ccccc6-c6ccccc65)cc43)n2)cc1 ; c1ccc(-c2nc(-c3cccc4ccccc34)nc(-n3c4ccccc4c4ccc(-c5ccc6c(c5)-c5ccccc5C65c6ccccc6-c6ccccc65)cc43)n2)cc1]

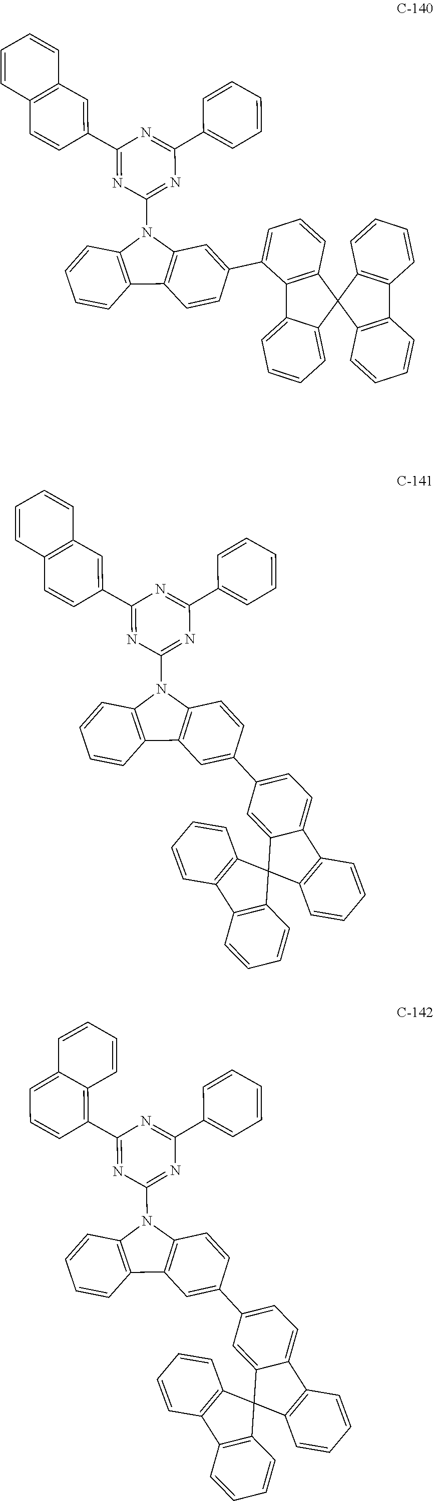 [SMILES: c1ccc(-c2nc(-c3ccc4ccccc4c3)nc(-n3c4ccccc4c4ccc(-c5cccc6c5-c5ccccc5C65c6ccccc6-c6ccccc65)cc43)n2)cc1 ; c1ccc(-c2nc(-c3ccc4ccccc4c3)nc(-n3c4ccccc4c4cc(-c5ccc6c(c5)C5(c7ccccc7-c7ccccc75)c5ccccc5-6)ccc43)n2)cc1 ; c1ccc(-c2nc(-c3cccc4ccccc34)nc(-n3c4ccccc4c4cc(-c5ccc6c(c5)C5(c7ccccc7-c7ccccc75)c5ccccc5-6)ccc43)n2)cc1]

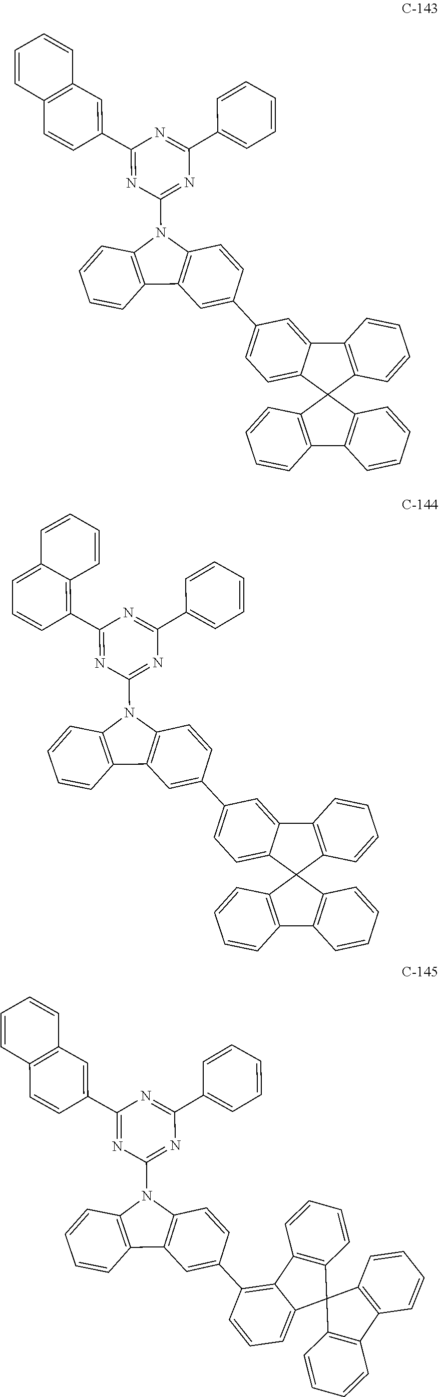 [SMILES: c1ccc(-c2nc(-c3ccc4ccccc4c3)nc(-n3c4ccccc4c4cc(-c5ccc6c(c5)-c5ccccc5C65c6ccccc6-c6ccccc65)ccc43)n2)cc1 ; c1ccc(-c2nc(-c3cccc4ccccc34)nc(-n3c4ccccc4c4cc(-c5ccc6c(c5)-c5ccccc5C65c6ccccc6-c6ccccc65)ccc43)n2)cc1 ; c1ccc(-c2nc(-c3ccc4ccccc4c3)nc(-n3c4ccccc4c4cc(-c5cccc6c5-c5ccccc5C65c6ccccc6-c6ccccc65)ccc43)n2)cc1]

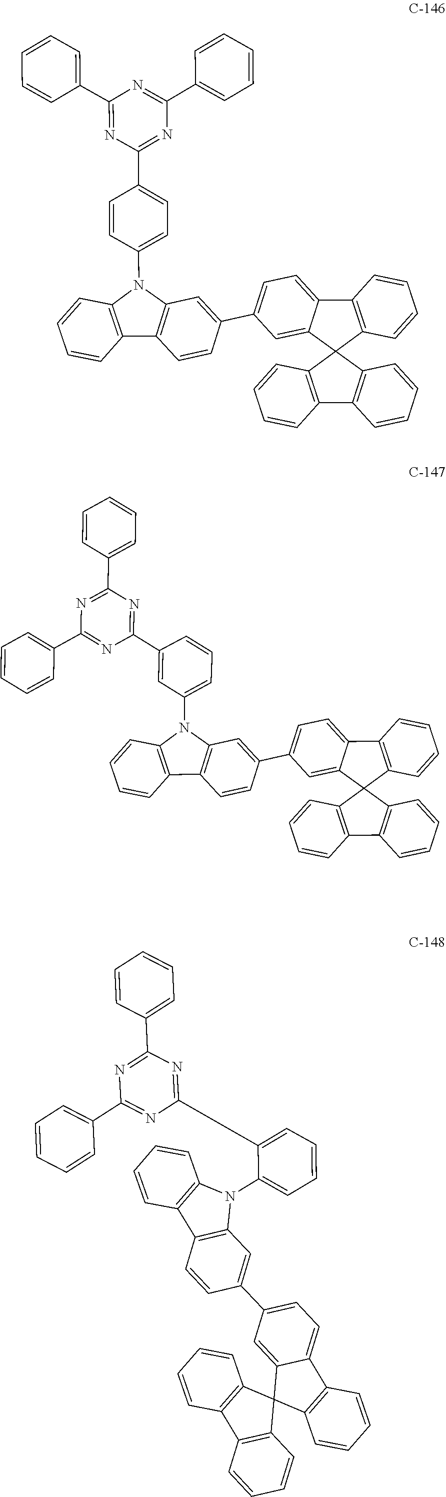 [SMILES: c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)cc3)n2)cc1 ; c1ccc(-c2nc(-c3ccccc3)nc(-c3cccc(-n4c5ccccc5c5ccc(-c6ccc7c(c6)C6(c8ccccc8-c8ccccc86)c6ccccc6-7)cc54)c3)n2)cc1 ; c1ccc(-c2nc(-c3ccccc3)nc(-c3ccccc3-n3c4ccccc4c4ccc(-c5ccc6c(c5)C5(c7ccccc7-c7ccccc75)c5ccccc5-6)cc43)n2)cc1]

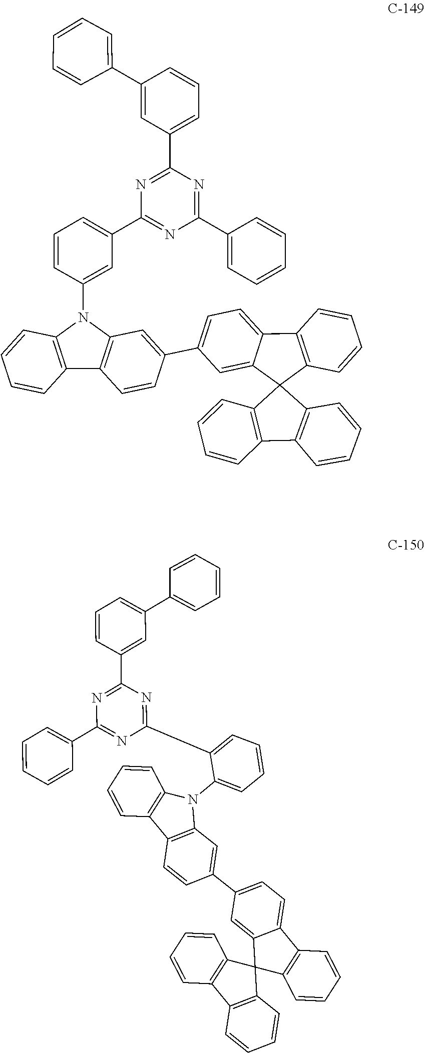 [SMILES: c1ccc(-c2cccc(-c3nc(-c4ccccc4)nc(-c4cccc(-n5c6ccccc6c6ccc(-c7ccc8c(c7)C7(c9ccccc9-c9ccccc97)c7ccccc7-8)cc65)c4)n3)c2)cc1 ; c1ccc(-c2cccc(-c3nc(-c4ccccc4)nc(-c4ccccc4-n4c5ccccc5c5ccc(-c6ccc7c(c6)C6(c8ccccc8-c8ccccc86)c6ccccc6-7)cc54)n3)c2)cc1]

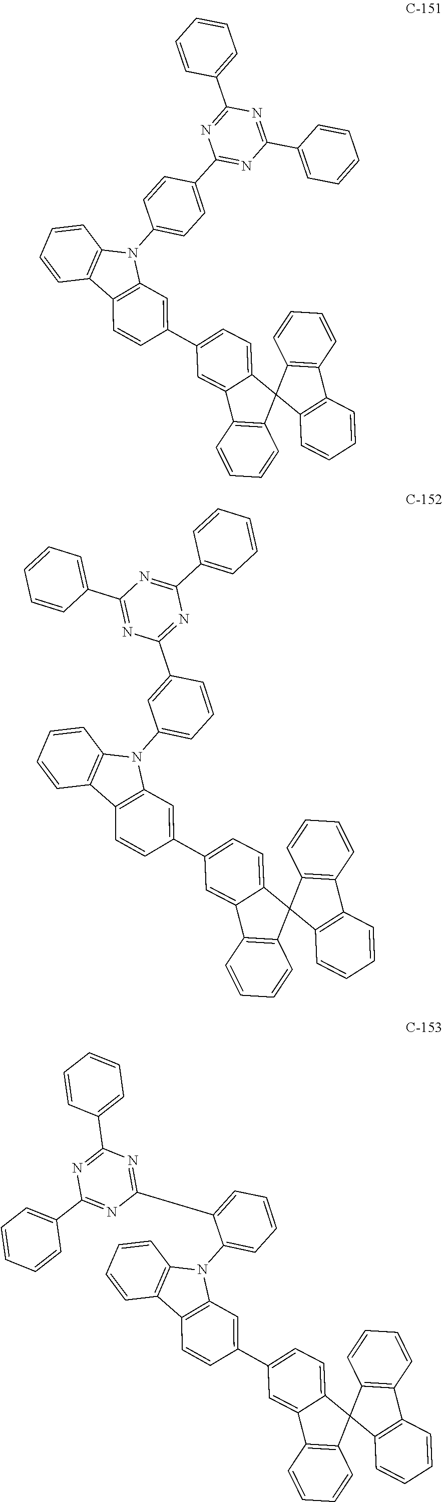

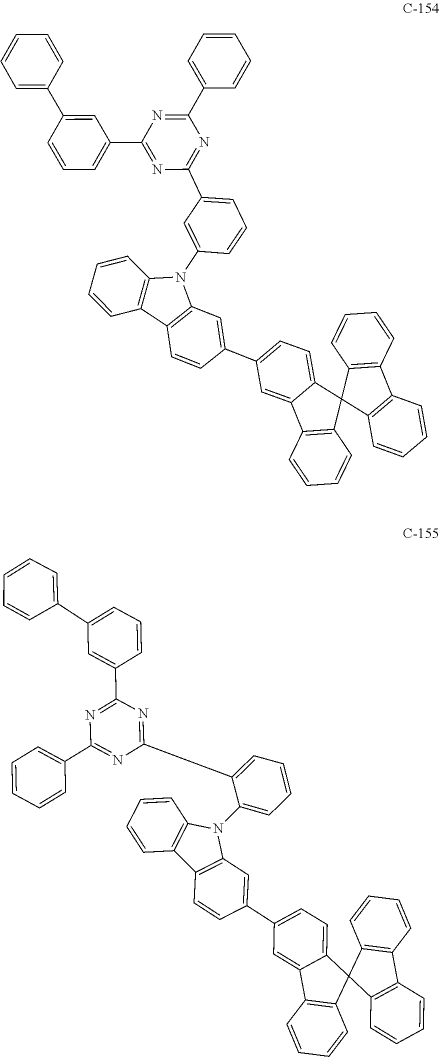 [SMILES: c1ccc(-c2cccc(-c3nc(-c4ccccc4)nc(-c4cccc(-n5c6ccccc6c6ccc(-c7ccc8c(c7)-c7ccccc7C87c8ccccc8-c8ccccc87)cc65)c4)n3)c2)cc1 ; c1ccc(-c2cccc(-c3nc(-c4ccccc4)nc(-c4ccccc4-n4c5ccccc5c5ccc(-c6ccc7c(c6)-c6ccccc6C76c7ccccc7-c7ccccc76)cc54)n3)c2)cc1]

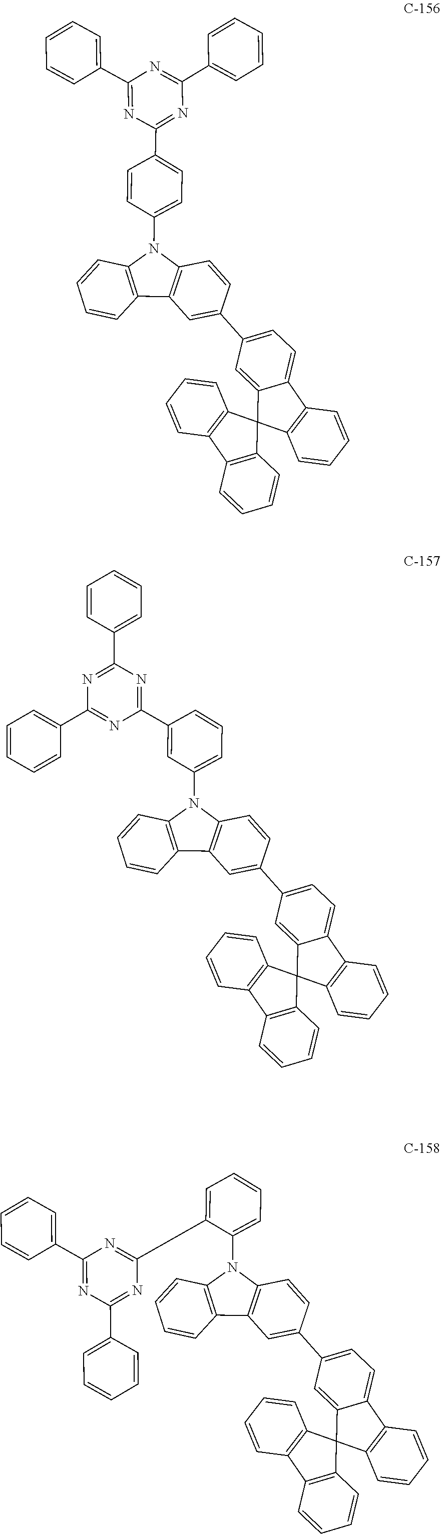 [SMILES: c1ccc(-c2nc(-c3ccccc3)nc(-c3ccc(-n4c5ccccc5c5cc(-c6ccc7c(c6)C6(c8ccccc8-c8ccccc86)c6ccccc6-7)ccc54)cc3)n2)cc1 ; c1ccc(-c2nc(-c3ccccc3)nc(-c3cccc(-n4c5ccccc5c5cc(-c6ccc7c(c6)C6(c8ccccc8-c8ccccc86)c6ccccc6-7)ccc54)c3)n2)cc1 ; c1ccc(-c2nc(-c3ccccc3)nc(-c3ccccc3-n3c4ccccc4c4cc(-c5ccc6c(c5)C5(c7ccccc7-c7ccccc75)c5ccccc5-6)ccc43)n2)cc1]

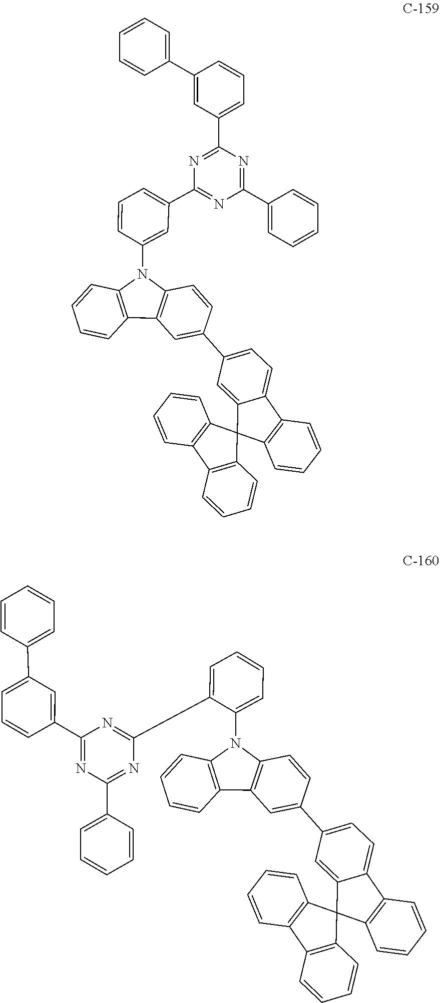 [SMILES: c1ccc(-c2cccc(-c3nc(-c4ccccc4)nc(-c4cccc(-n5c6ccccc6c6cc(-c7ccc8c(c7)C7(c9ccccc9-c9ccccc97)c7ccccc7-8)ccc65)c4)n3)c2)cc1 ; c1ccc(-c2cccc(-c3nc(-c4ccccc4)nc(-c4ccccc4-n4c5ccccc5c5cc(-c6ccc7c(c6)C6(c8ccccc8-c8ccccc86)c6ccccc6-7)ccc54)n3)c2)cc1]

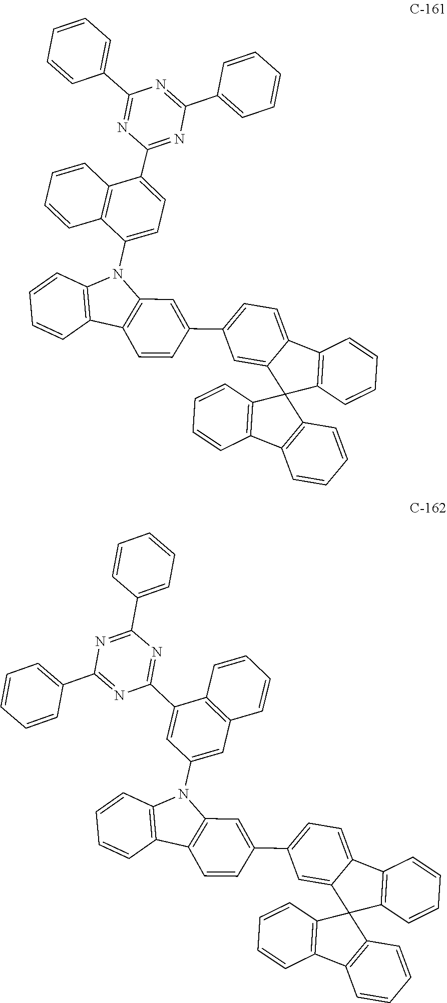 [SMILES: c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)c4ccccc34)n2)cc1 ; c1ccc(-c2nc(-c3ccccc3)nc(-c3cc(-n4c5ccccc5c5ccc(-c6ccc7c(c6)C6(c8ccccc8-c8ccccc86)c6ccccc6-7)cc54)cc4ccccc34)n2)cc1]

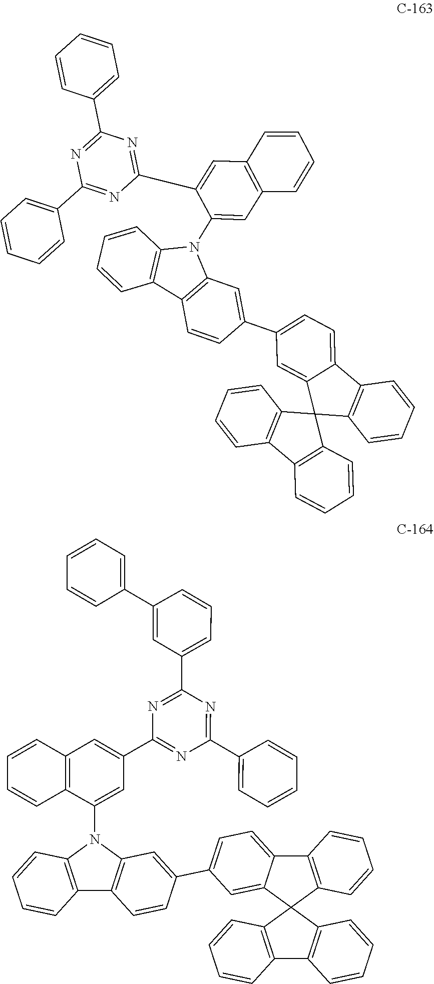 [SMILES: c1ccc(-c2nc(-c3ccccc3)nc(-c3cc4ccccc4cc3-n3c4ccccc4c4ccc(-c5ccc6c(c5)C5(c7ccccc7-c7ccccc75)c5ccccc5-6)cc43)n2)cc1 ; c1ccc(-c2cccc(-c3nc(-c4ccccc4)nc(-c4cc(-n5c6ccccc6c6ccc(-c7ccc8c(c7)C7(c9ccccc9-c9ccccc97)c7ccccc7-8)cc65)c5ccccc5c4)n3)c2)cc1]

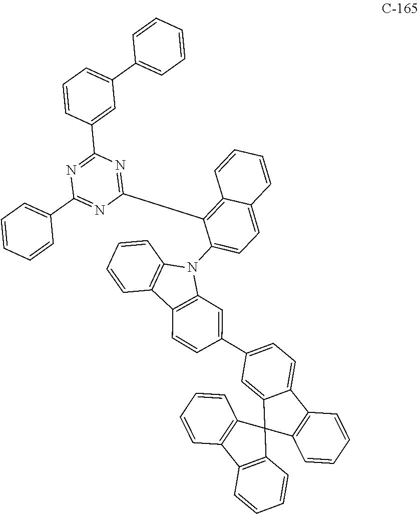 [SMILES: c1ccc(-c2cccc(-c3nc(-c4ccccc4)nc(-c4c(-n5c6ccccc6c6ccc(-c7ccc8c(c7)C7(c9ccccc9-c9ccccc97)c7ccccc7-8)cc65)ccc5ccccc45)n3)c2)cc1]

Hereinafter, an organic electroluminescent device to which the aforementioned plurality of host materials and/or organic electroluminescent compound is (are) applied, will be described.

The organic electroluminescent device according to one embodiment includes a first electrode; a second electrode; and at least one organic layer(s) interposed between the first electrode and the second electrode. The organic layer may include a light-emitting layer, and the light-emitting layer may comprise a plurality of host materials comprising at least one first host compound represented by Formula 1 and at least one second host compound represented by Formula 2. Wherein, the weight ratio of the first host compound to the second host compound may be in the range of about 1:99 to about 99:1, preferably about 10:90 to about 90:10, more preferably about 30:70 to about 70:30, more preferably about 40:60 to about 60:40, and even more preferably about 50:50 in the light-emitting layer.

According to one embodiment, the organic electroluminescent material of the present disclosure comprises at least one compound(s) of compounds C-1 to C-169, which is a first host compound, and at least one compound(s) of compounds H2-1 to H2-290, which is a second host compound. The plurality of host materials may be included in the same organic layer, for example the same light-emitting layer, or may be included in different light-emitting layers, respectively.

According to another embodiment, the organic electroluminescent material of the present disclosure comprises an organic electroluminescent compound represented by Formula 1′ alone or in combination of two or more, and such organic electroluminescent material may be included in an organic layer of an organic electroluminescent device. For example: an electron transport layer, an electron buffer layer, a hole blocking layer, or a light emitting layer.

The organic layer may further comprise at least one layer selected from a hole injection layer, a hole auxiliary layer, a light-emitting auxiliary layer, an electron transport layer, an electron injection layer, an interlayer, a hole blocking layer, an electron blocking layer and an electron buffer layer, in addition to the light-emitting layer and the hole transport layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Also, the electron transport layer, the electron injection layer, the electron buffer layer, or the hole blocking layer may contain the azine-based compound as an electron transport material, an electron injection material, an electron buffer material, or a hole blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the plurality of host materials according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X}(1≤X≤2), AlO_{X}(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation; thus, it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

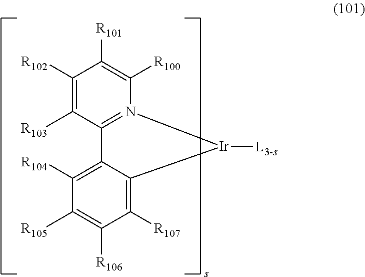

In Formula 101,
L is any one selected from the following structures 1 to 3;

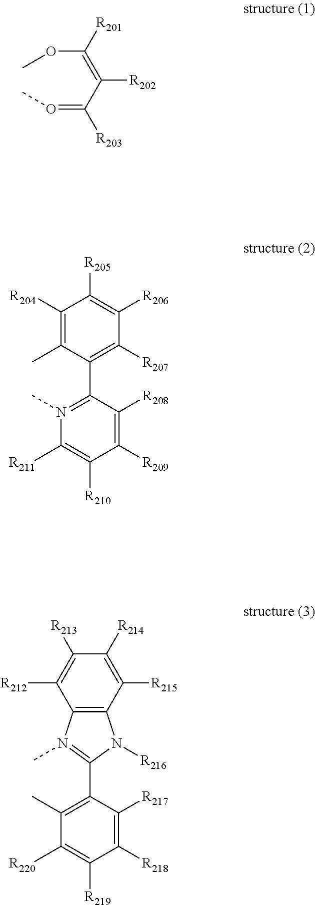

in structures 1 to 3,
R₁₀₀ to R₁₀₃ each independently represent hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to the adjacent substituents to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or may be linked to an adjacent substituent(s) to form a substituted or unsubstituted ring(s), for example, to form a substituted or unsubstituted ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to an adjacent substituent(s) to form a substituted or unsubstituted ring(s); and
s represents an integer of 1 to 3.

Specifically, the specific examples of the dopant compound include the following, but are not limited thereto.

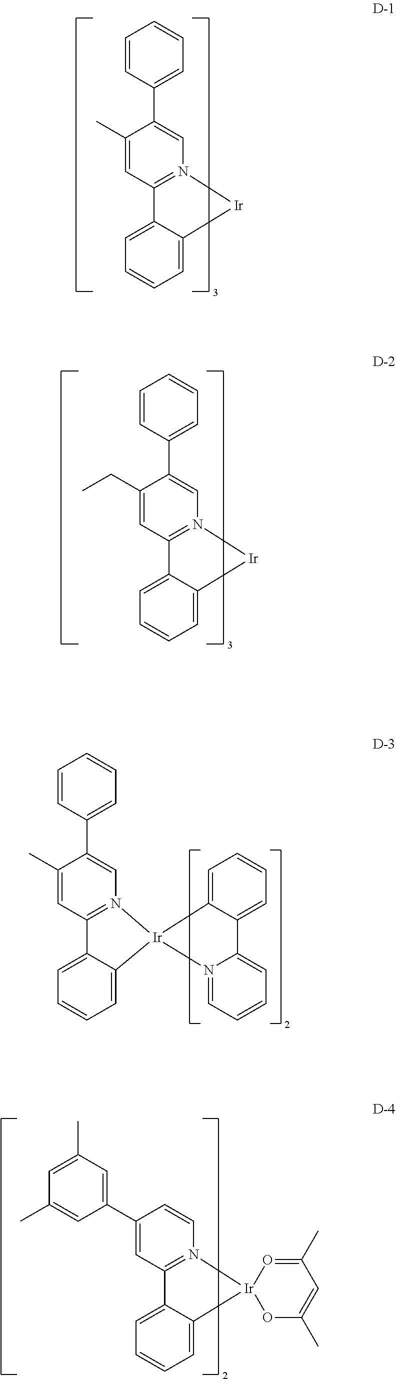

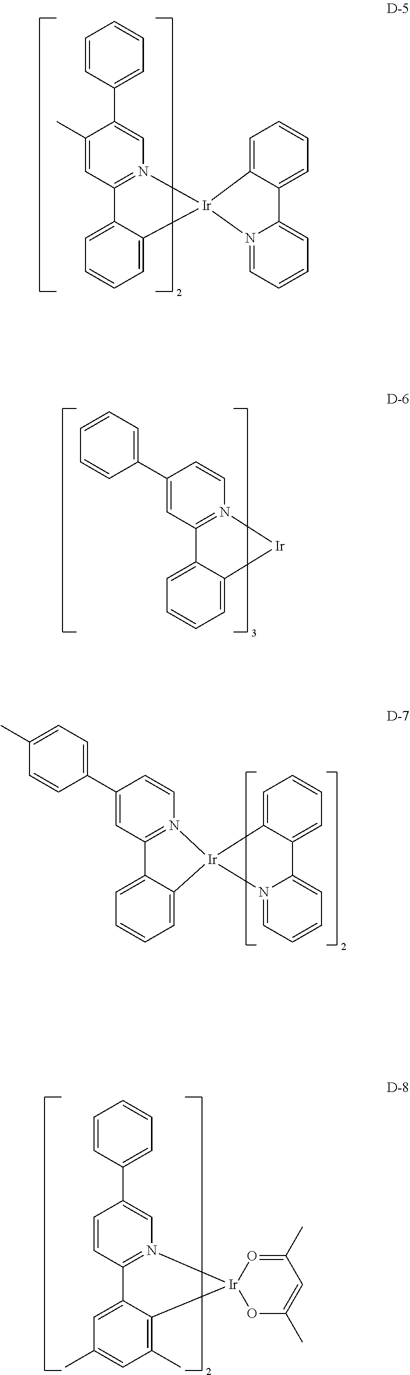

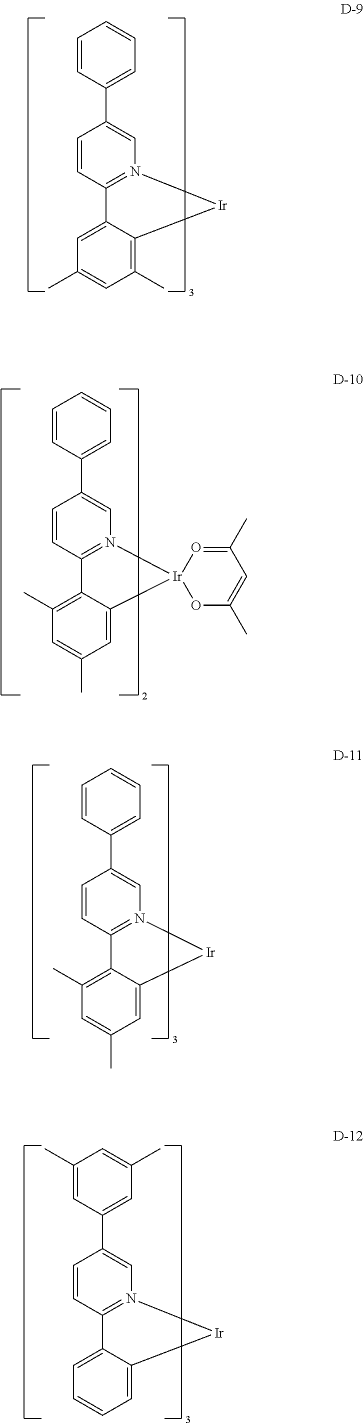

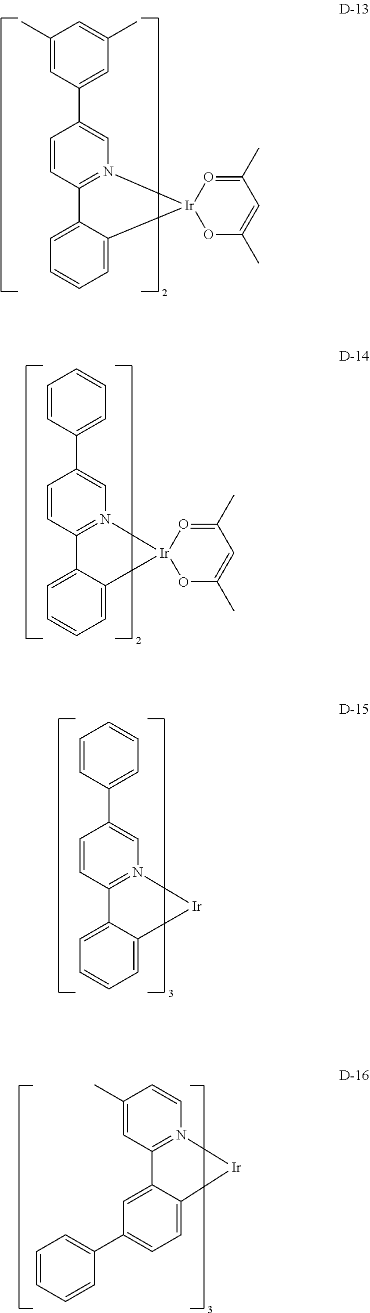

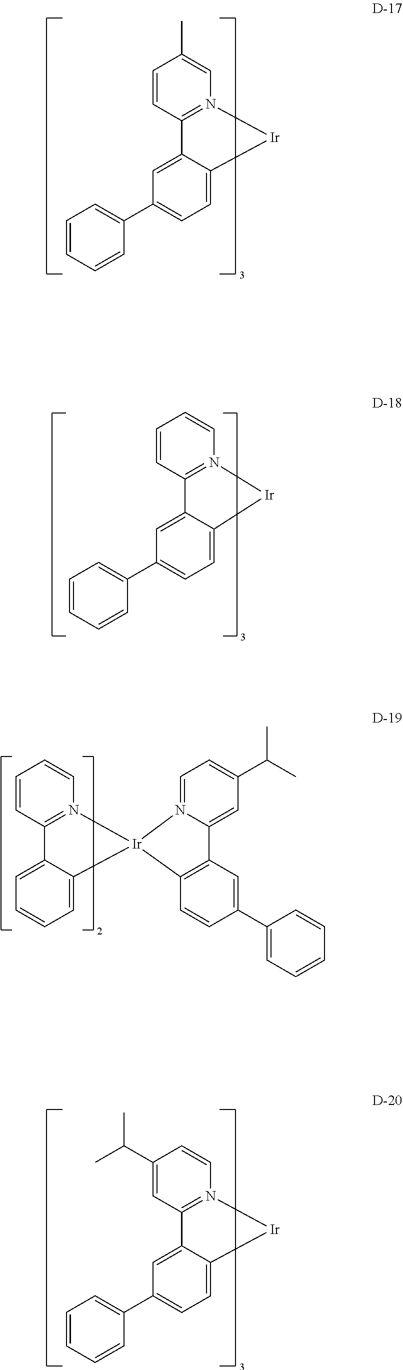

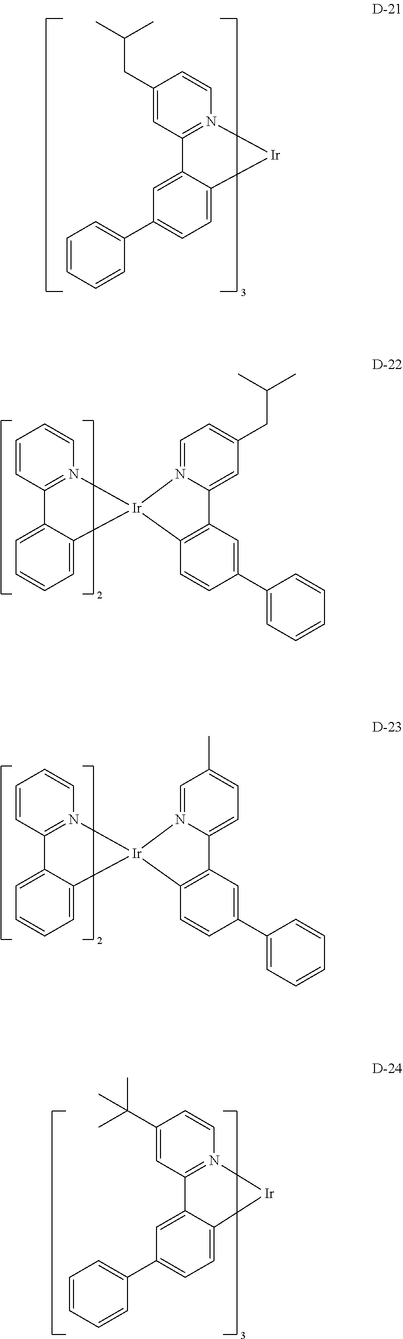

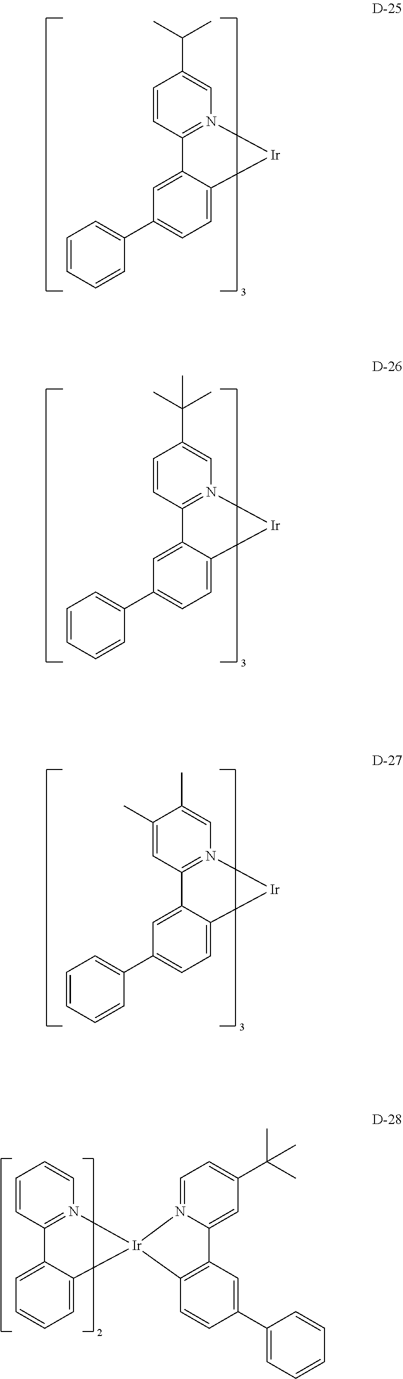

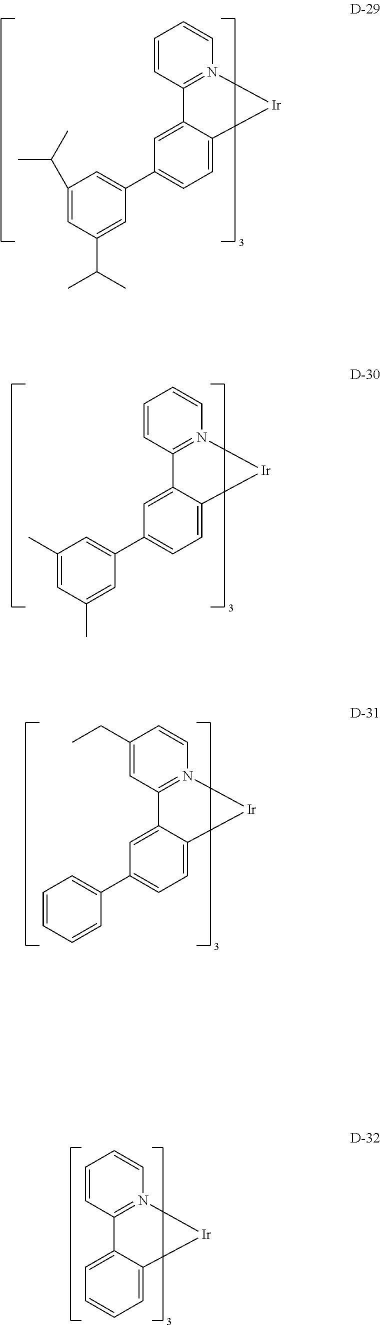

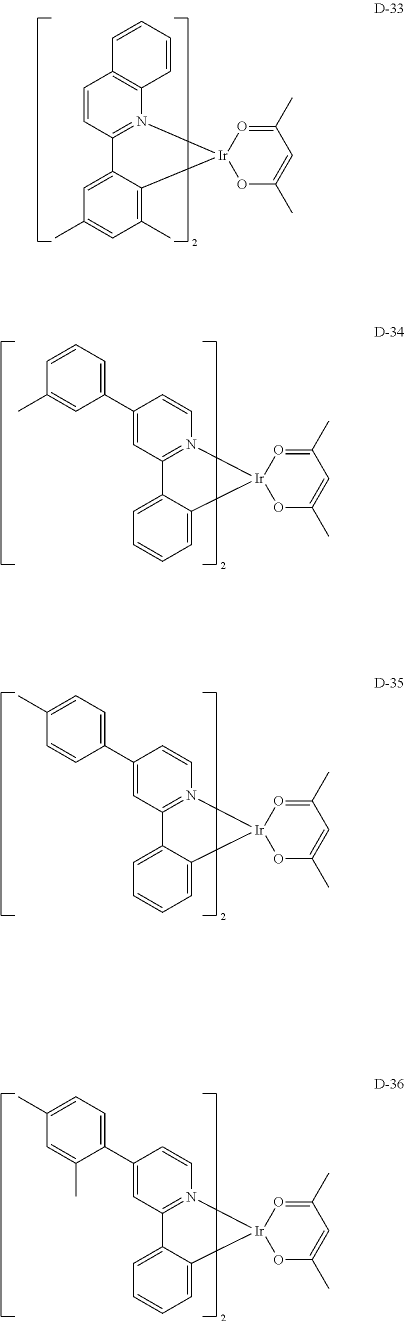

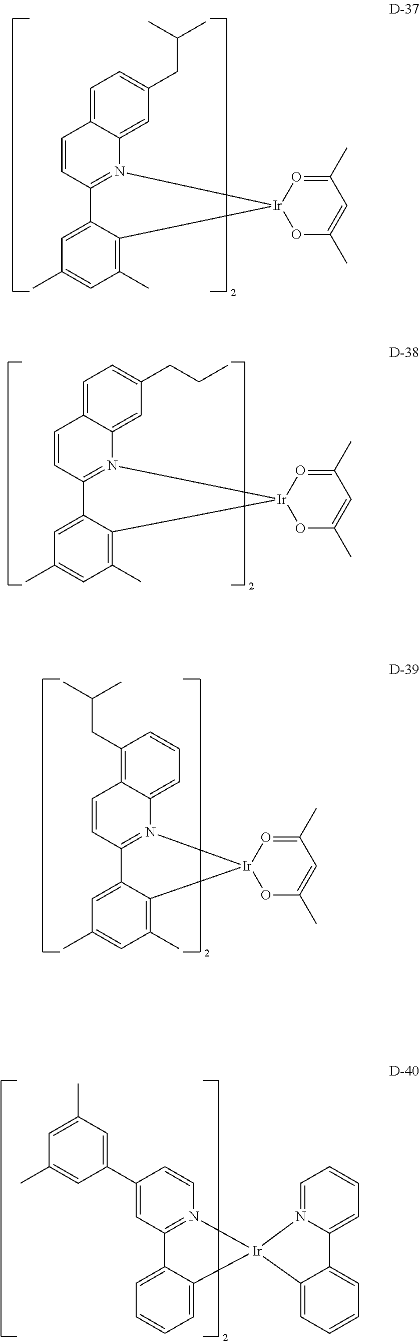

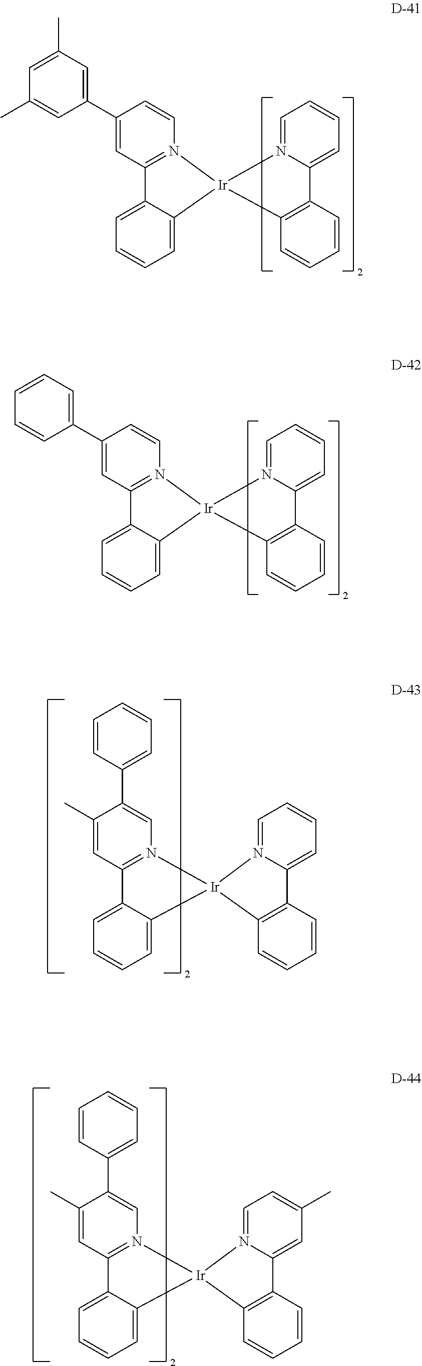

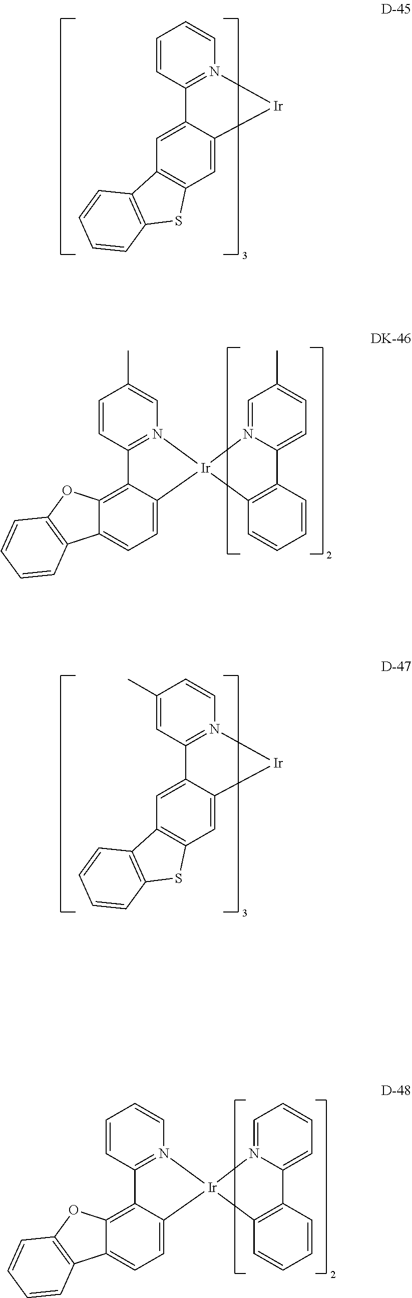

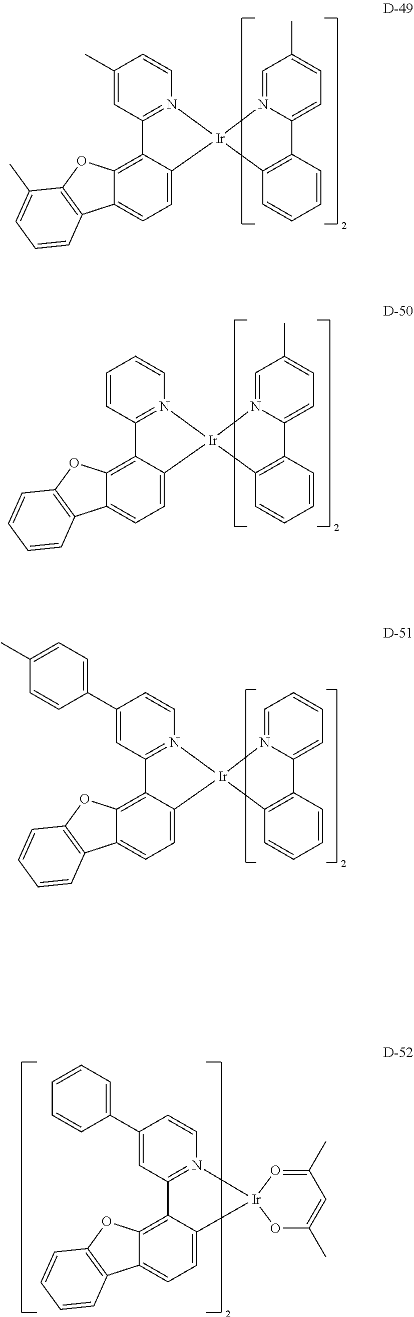

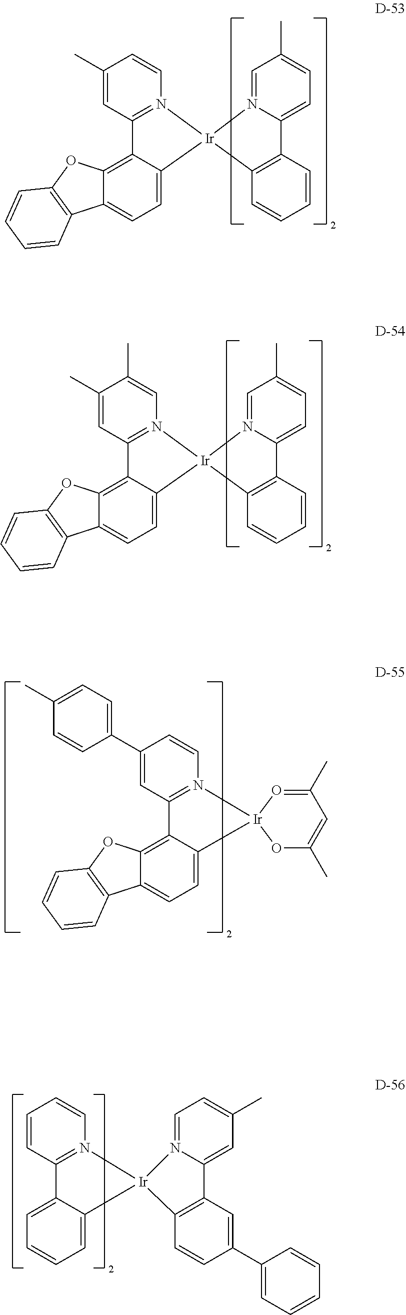

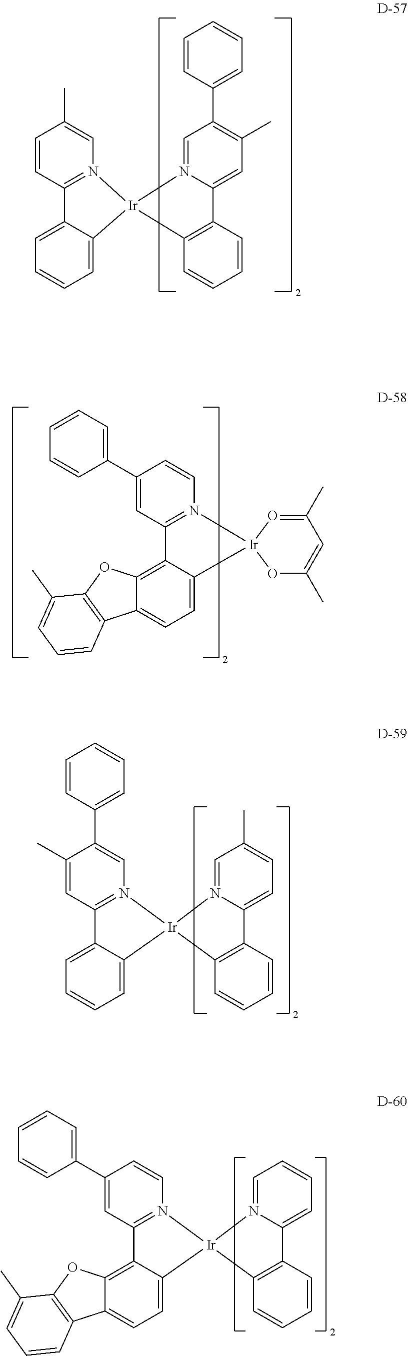

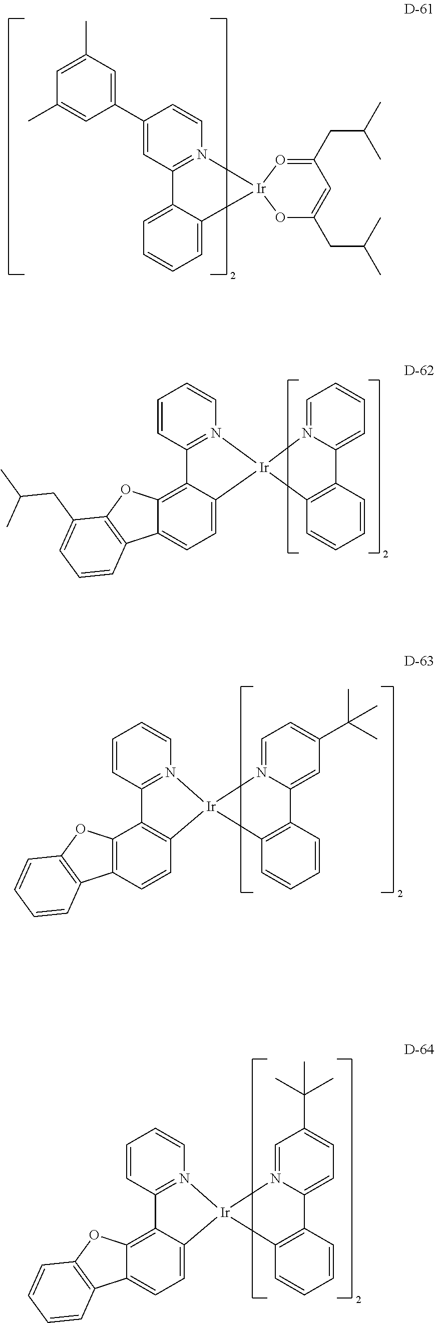

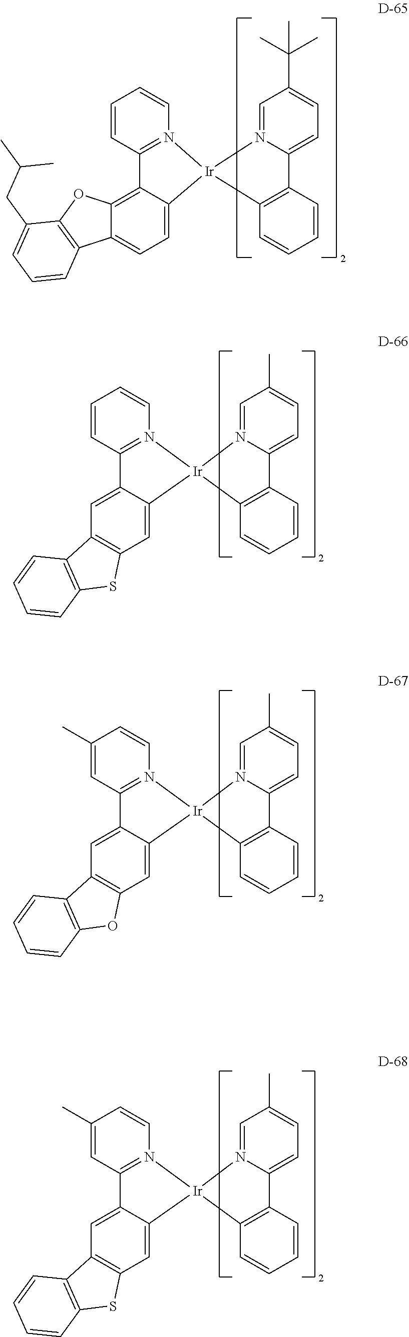

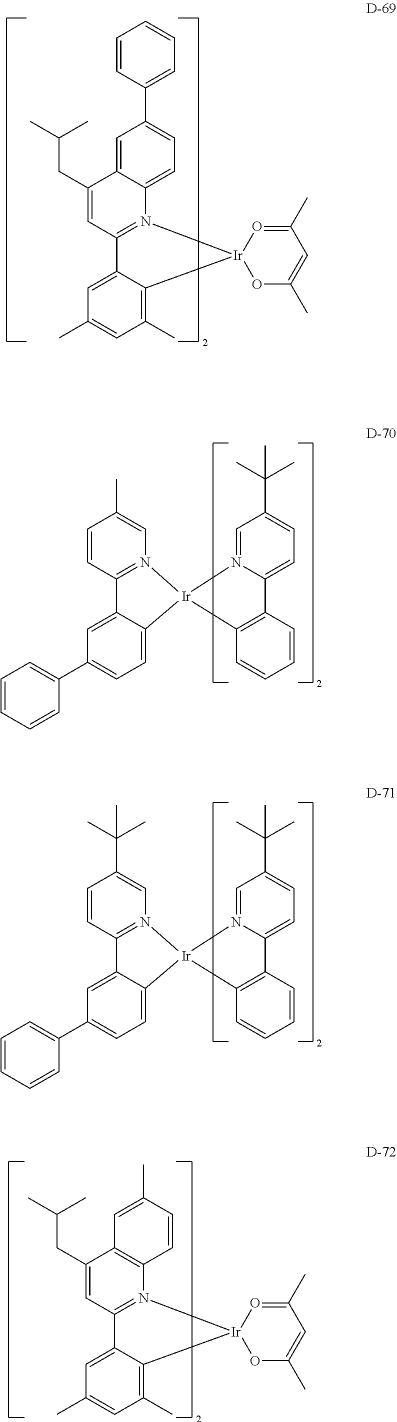

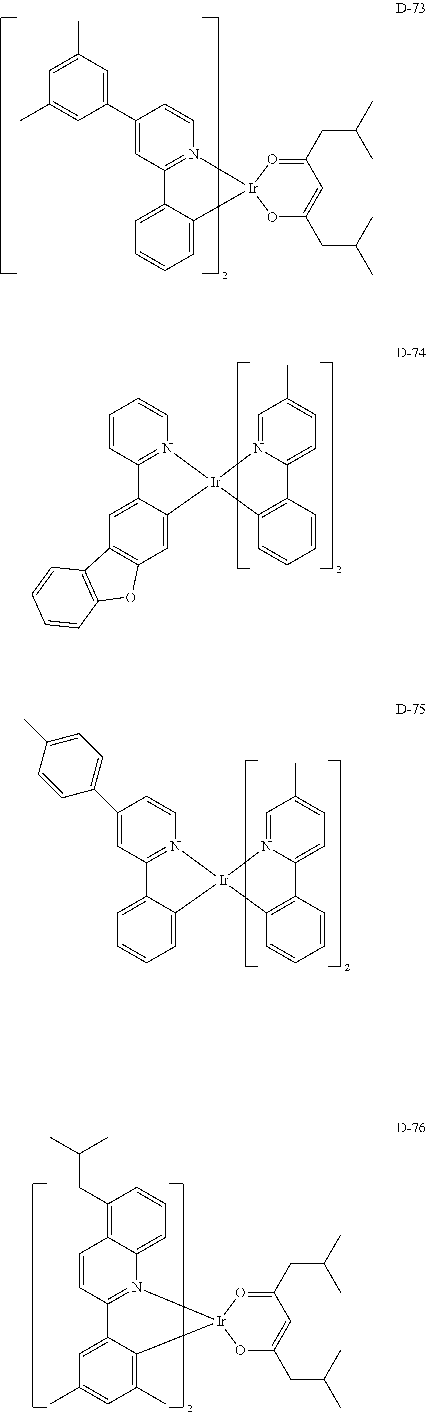

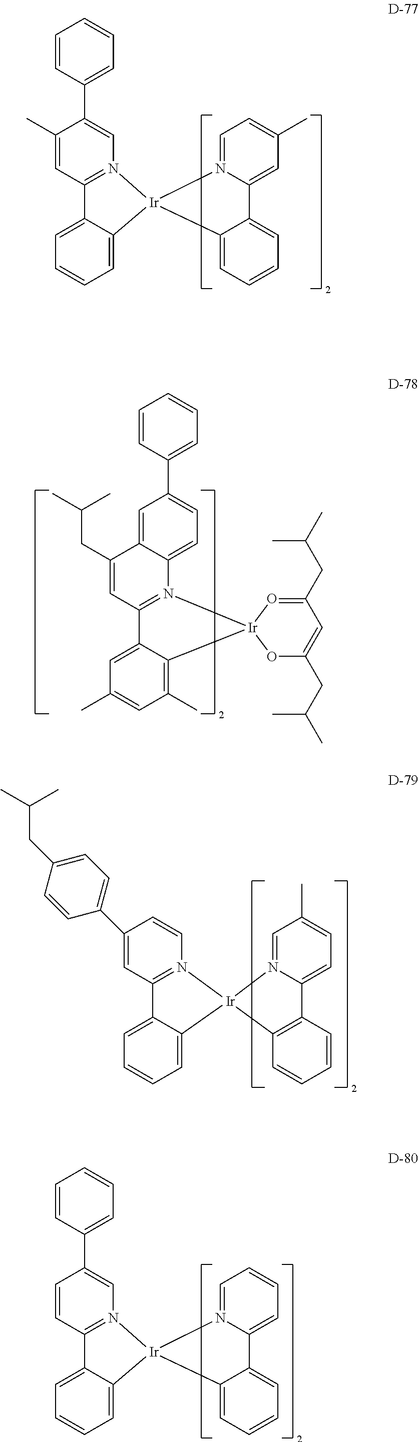

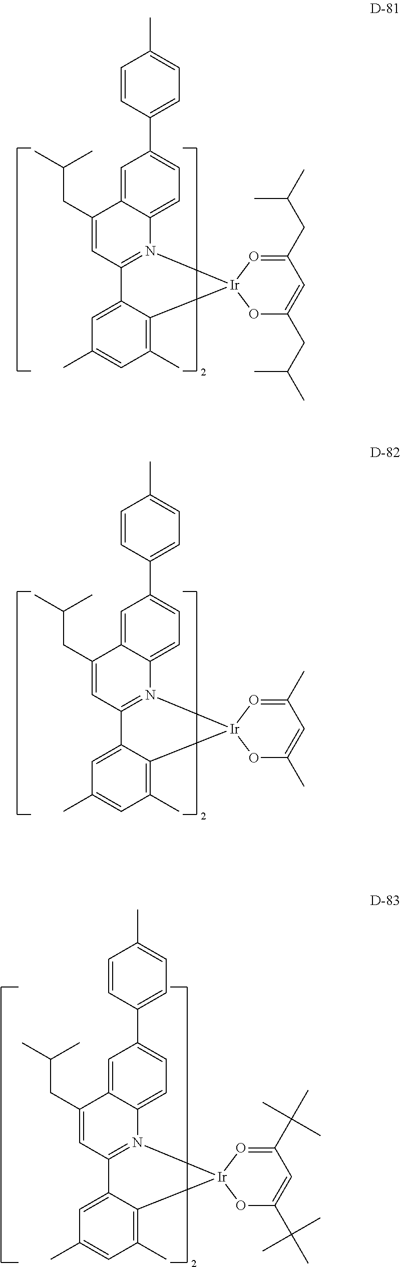

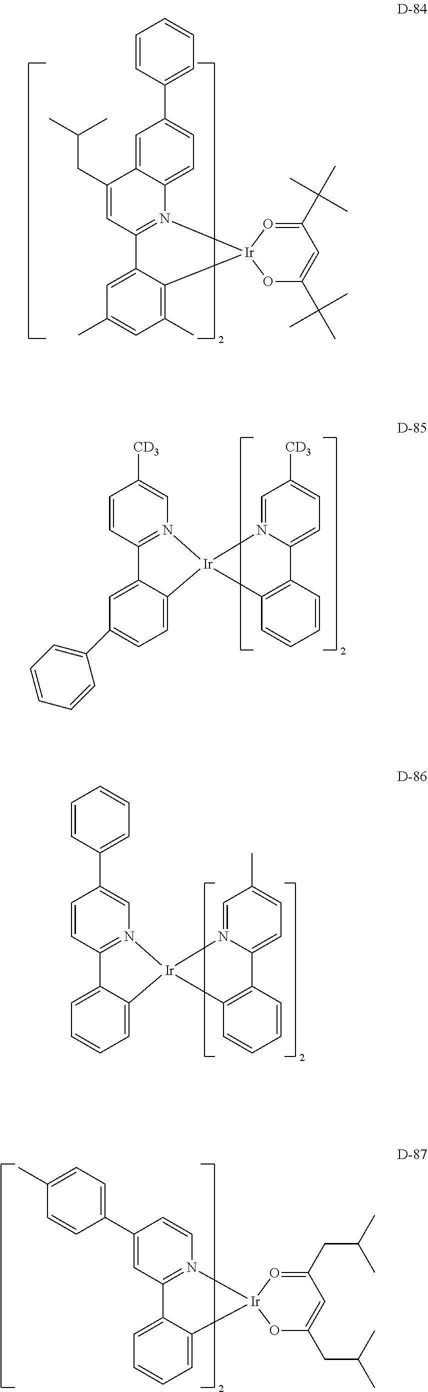

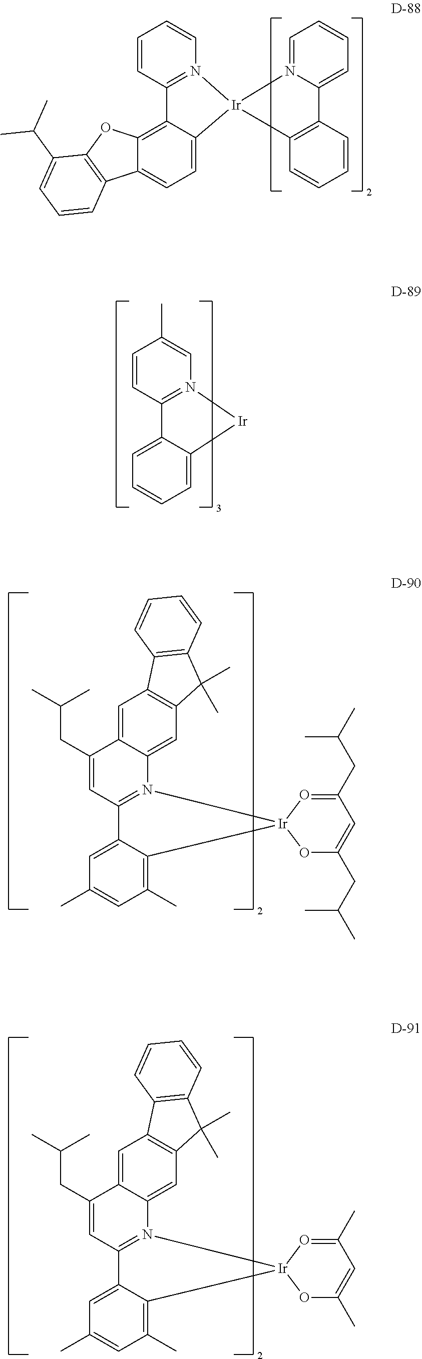

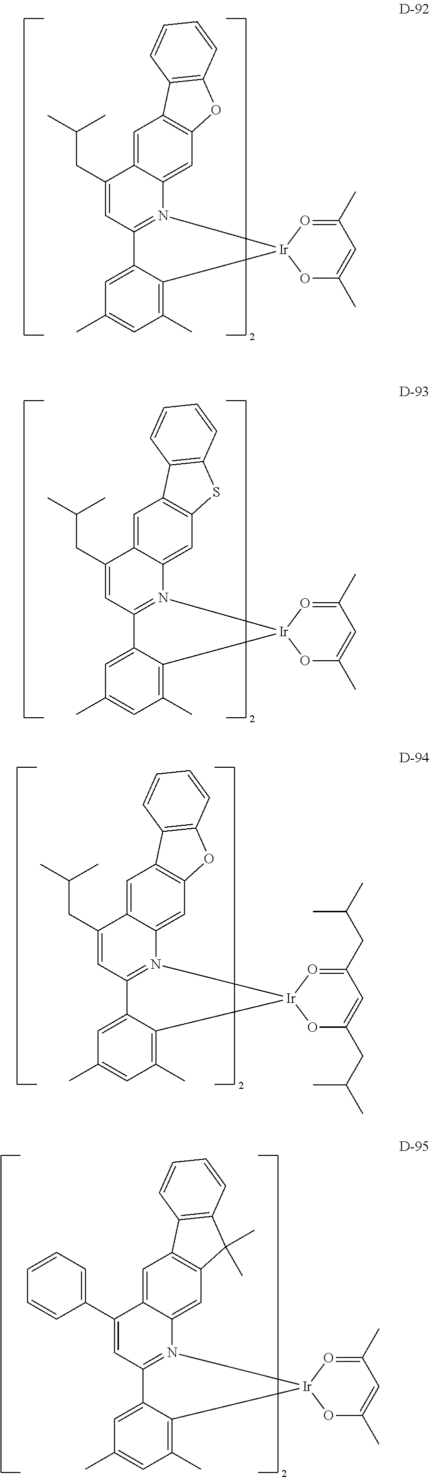

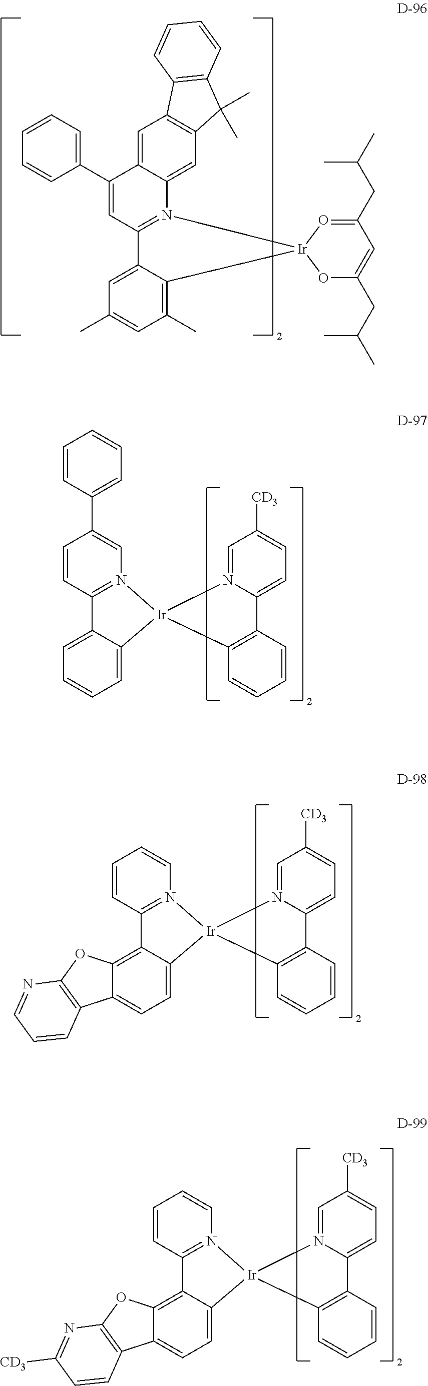

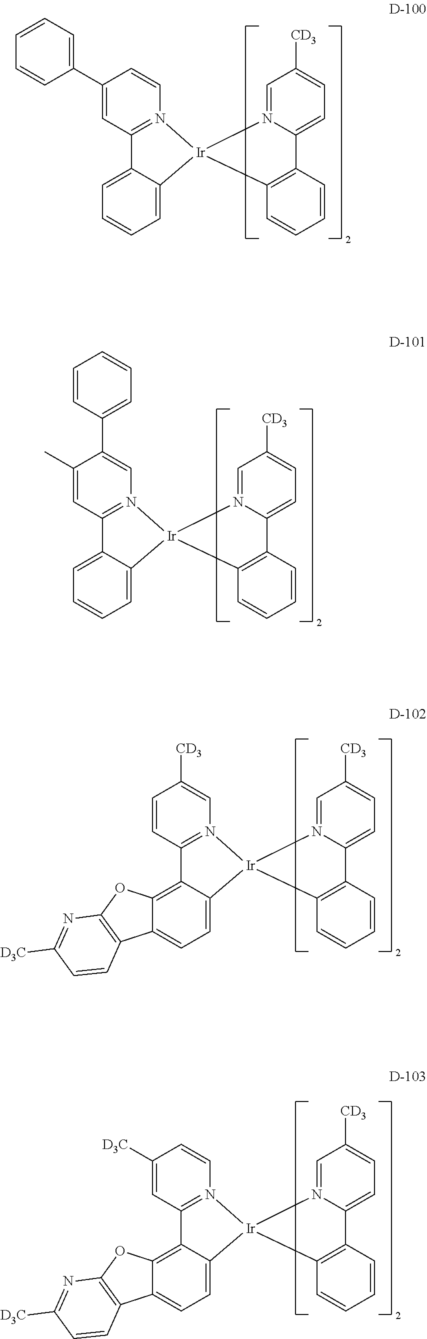

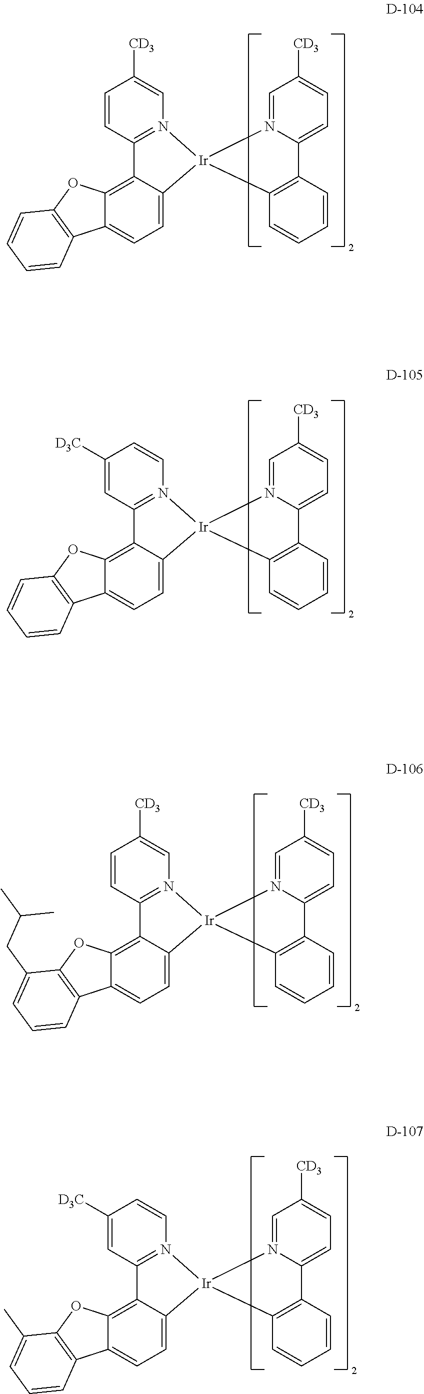

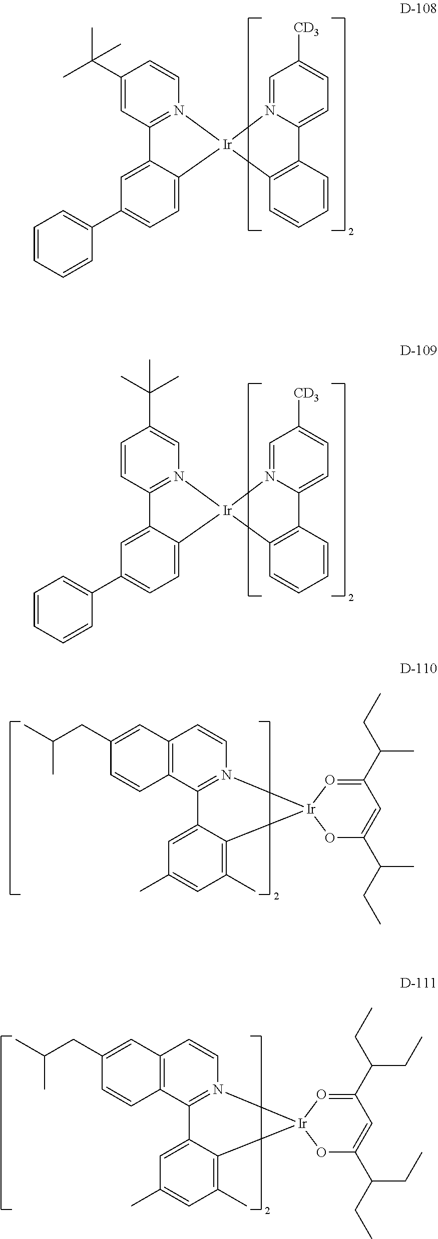

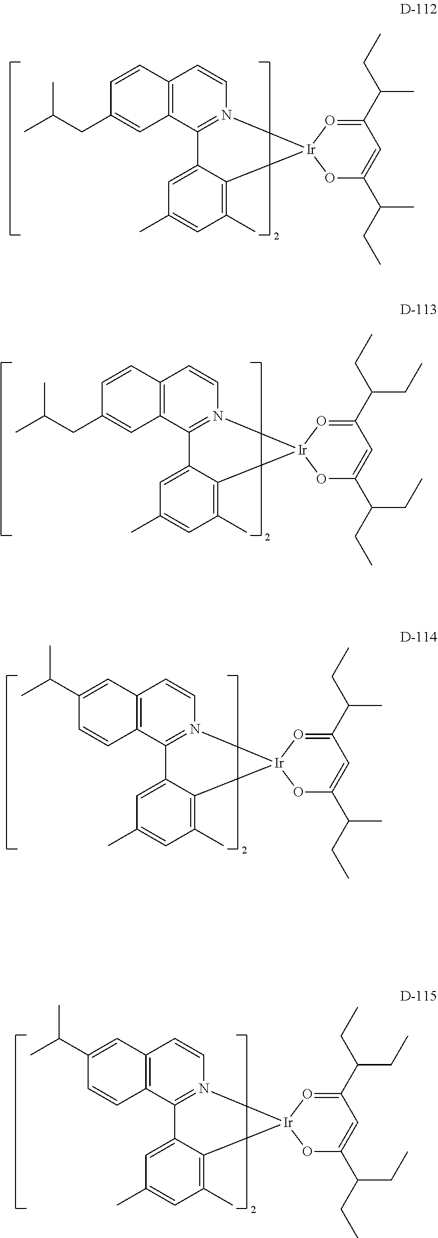

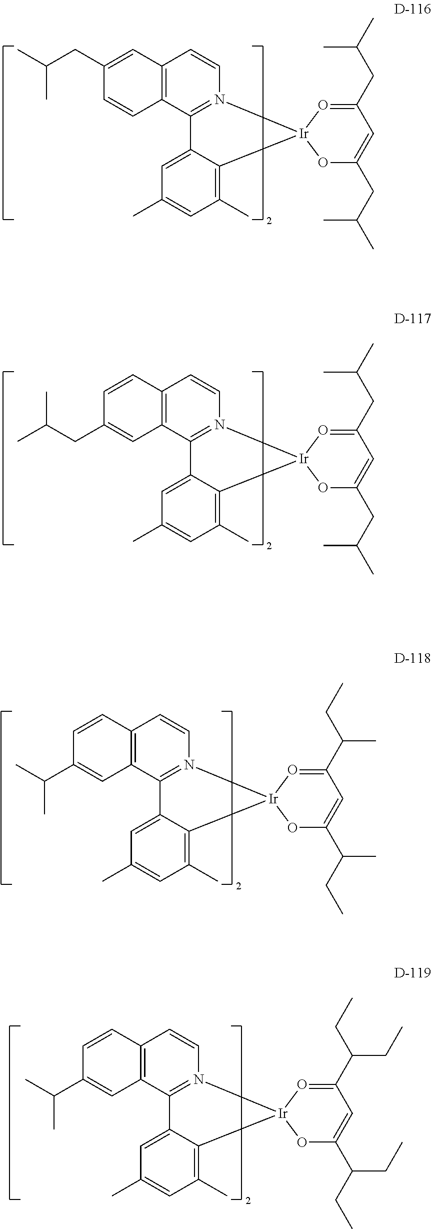

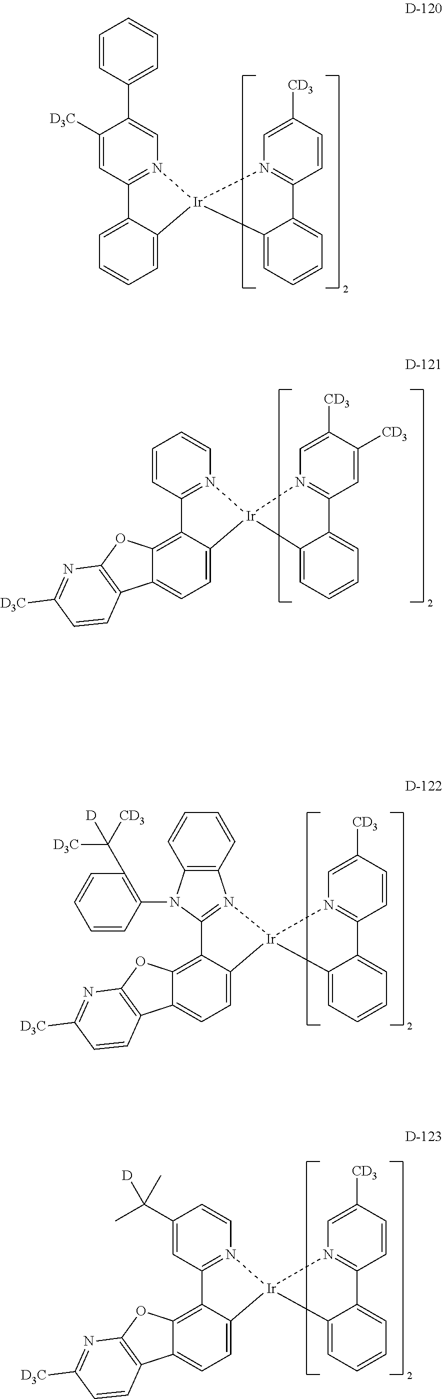

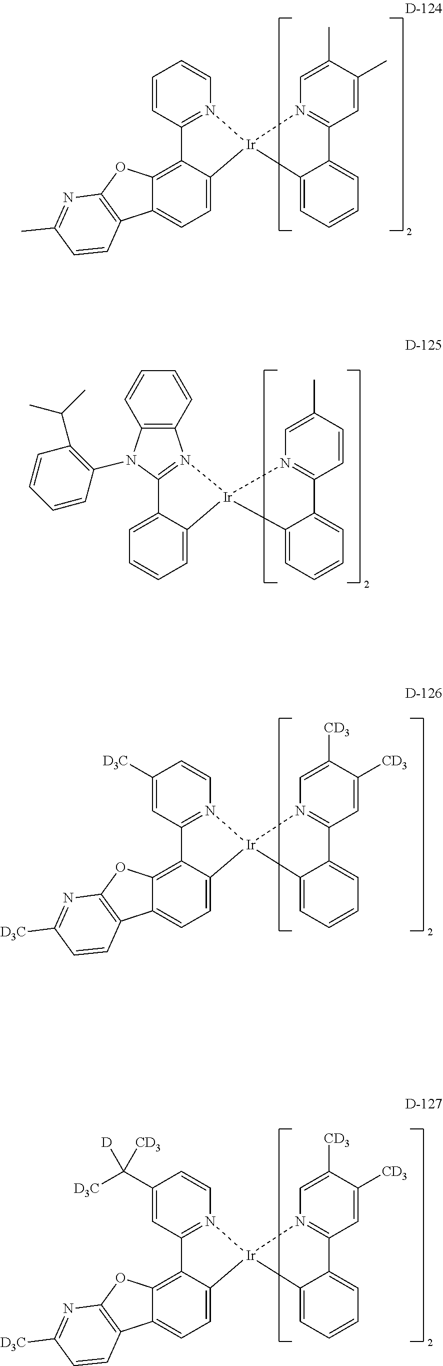

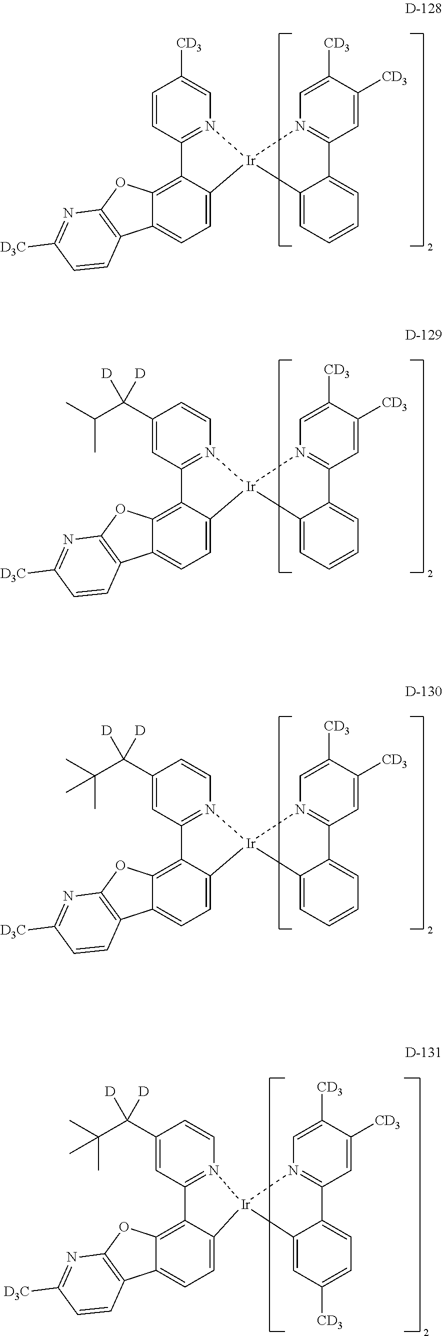

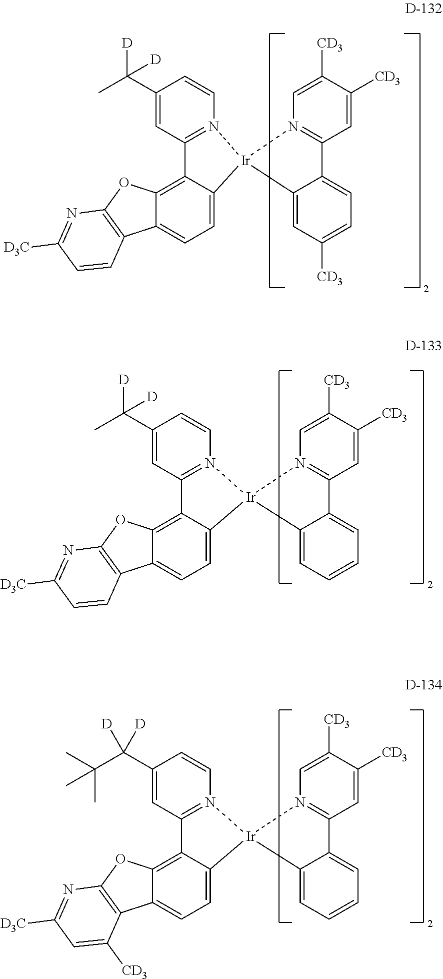

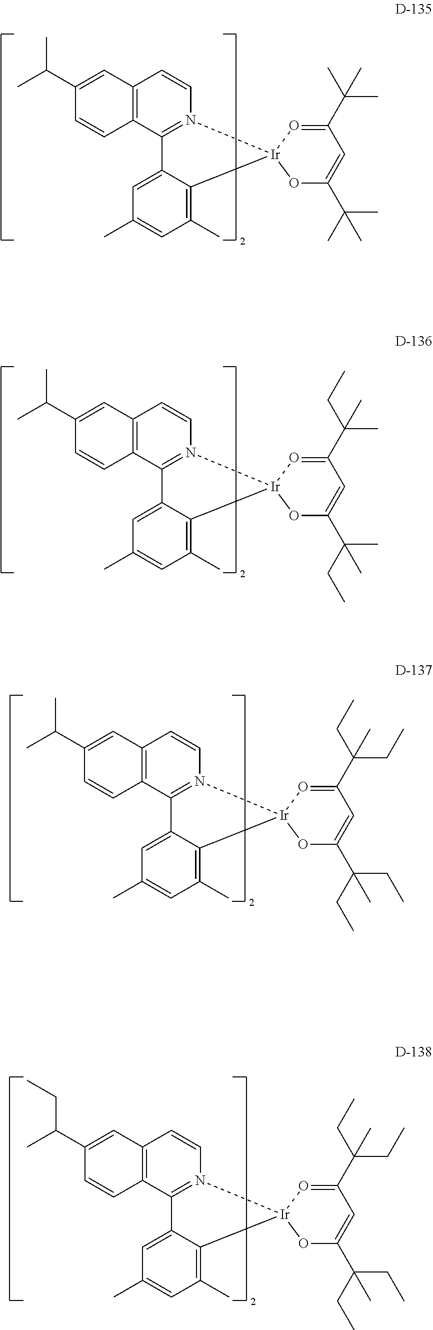

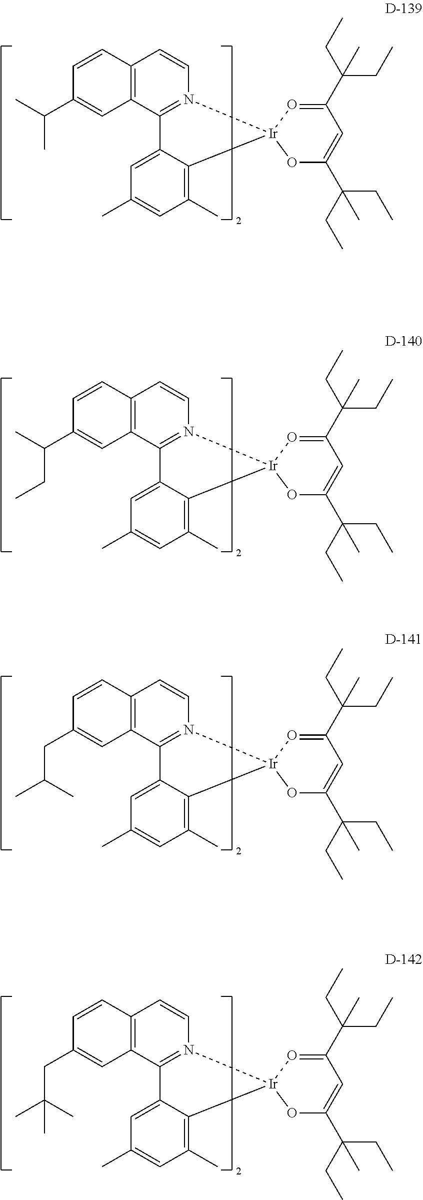

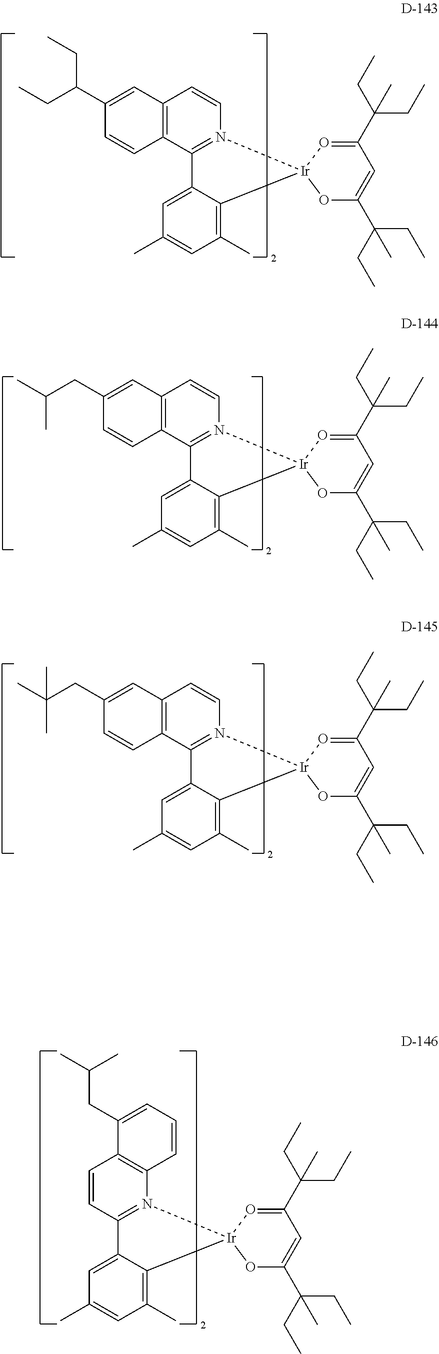

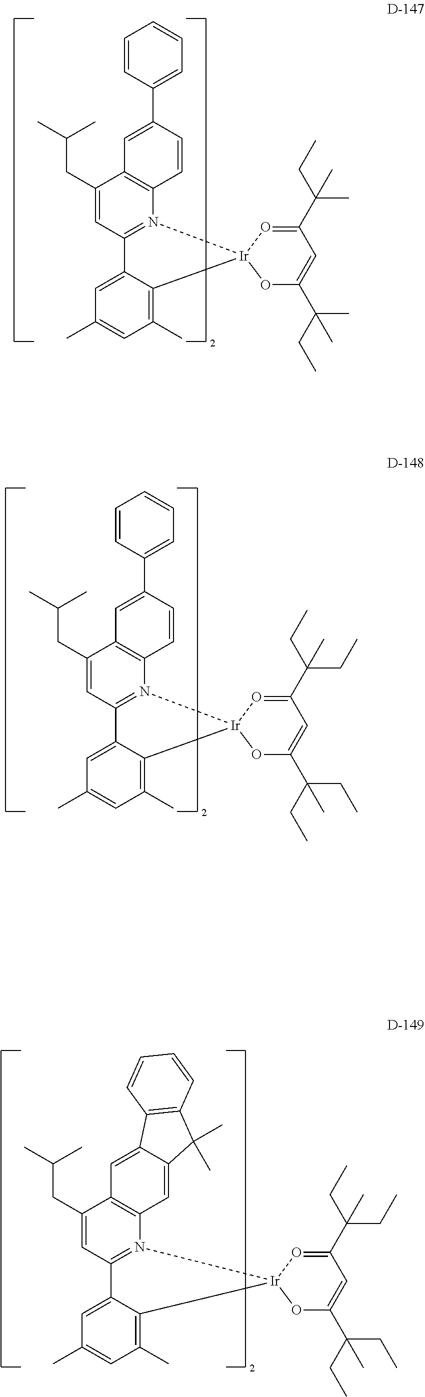

In order to form each layer of the organic electroluminescent device of the present disclosure, dry film-forming methods such as vacuum evaporation, sputtering, plasma, ion plating methods, etc., or wet film-forming methods such as spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host compound and the second host compound according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials and to perform mixed deposition; and the mixed deposition is a mixed deposition method in which two or more materials are mixed in one crucible source before deposition, and then a current is applied to one cell to evaporate the materials.

According to one embodiment, when the first host compound and the second host compound exist in the same layer or different layers in the organic electroluminescent device, the layers by the two host compounds may be separately formed. For example, after depositing the first host compound, a second host compound may be deposited.

According to one embodiment, the present disclosure can provide display devices comprising a plurality of host materials comprising a first host compound represented by Formula 1 and a second host compound represented by Formula 2. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of the host materials according to the present disclosure will be explained with reference to the synthesis method of a representative compound or intermediate compound in order to understand the present disclosure in detail.

### [Example 1] Synthesis of Compound C-62

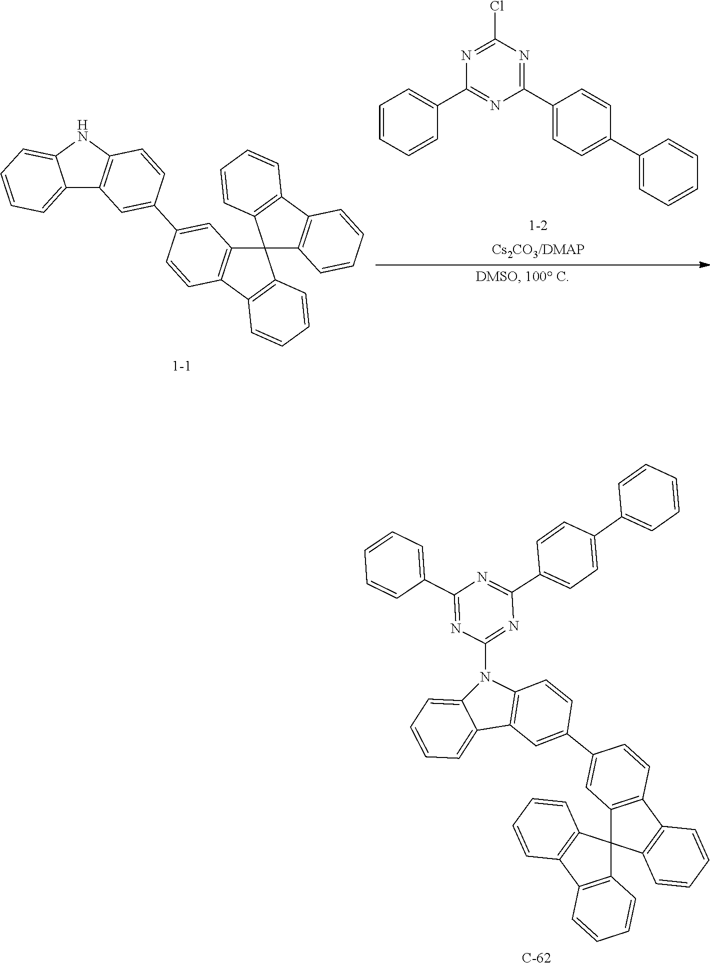

Compound 1-1 (3 g, 6.22 mmol), Compound 1-2 (2.6 g, 7.4 mmol), cesium carbonate (Cs₂CO₃) (2 g, 6.22 mmol), 4-(dimethylamino)pyridine (DMAP) (380 mg, 3.11 mmol), and 31 mL of dimethyl sulfoxide (DMSO) were added to a flask and dissolved, and then refluxed at 100° C. for 2 hours. When the reaction was completed, the mixture was cooled to room temperature, and H₂O was added to the resulting solid. Next, it was stirred for 30 minutes, followed by filtering, and then separated by column chromatography to obtain Compound C-62 (4 g, yield: 82%).

| | MW | M.P |
|---|---|---|
| C-62 | 788.95 | 323.5° C. |

### [Example 2] Synthesis of Compound C-147

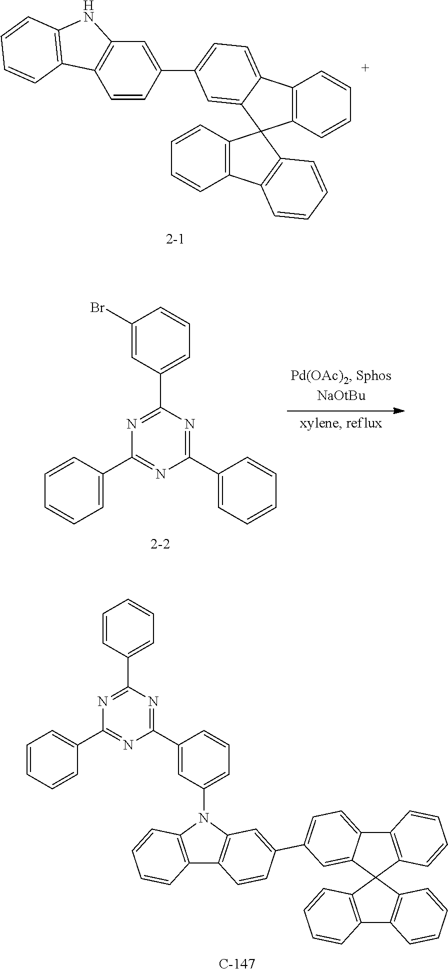 [SMILES: c1ccc2c(c1)-c1ccccc1C21c2ccccc2-c2ccc(-c3ccc4c(c3)[nH]c3ccccc34)cc21 ; Brc1cccc(-c2nc(-c3ccccc3)nc(-c3ccccc3)n2)c1 ; c1ccc(-c2nc(-c3ccccc3)nc(-c3cccc(-n4c5ccccc5c5ccc(-c6ccc7c(c6)C6(c8ccccc8-c8ccccc86)c6ccccc6-7)cc54)c3)n2)cc1]

Compound 2-1 (5 g, 10.38 mmol), Compound 2-2 (4.8 g, 12.45 mmol), Pd(OAc)₂ (233 mg, 1.03 mmol), S-Phos (852 mg, 2.07 mmol), NaOtBu (1.9 g, 20.76 mmol) in a flask were dissolved in 103 mL of o-xylene, and then stirred under reflux for 4 hours. When the reaction was completed, the mixture was cooled to room temperature, and H₂O was added to the resulting solid. Next, it was stirred for 30 minutes, followed by filtering, and then separated by column chromatography to obtain Compound C-147 (2.7 g, yield: 33%).

| | MW | M.P |
|---|---|---|
| C-147 | 788.95 | 166.3° C. |

### [Example 3] Synthesis of Compound C-1

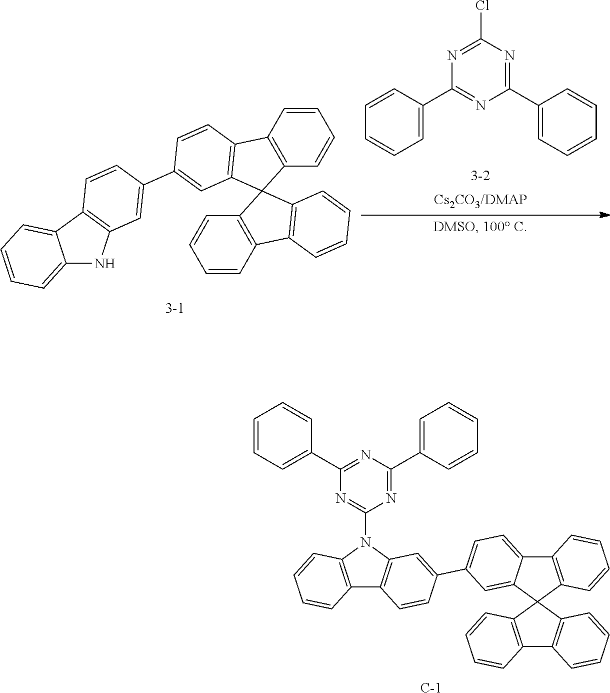

Compound 3-1 (7 g, 14.53 mmol), Compound 3-2 (4.3 g, 15.99 mmol), Cs₂CO₃ (4.7 g, 614.53 mmol), DMAP (890 mg, 7.27 mmol), and 72 mL of DMSO were added to a flask and dissolved, and then refluxed at 100° C. for 2 hours. When the reaction was completed, the mixture was cooled to room temperature, and H₂O was added to the resulting solid. Next, it was stirred for 30 minutes, followed by filtering, and then separated by column chromatography to obtain Compound C-1 (3.1 g, yield: 30%).

| | MW | M.P |
|---|---|---|
| C-1 | 712.86 | 375° C. |

Hereinafter, the preparation method of an organic electroluminescent device comprising the plurality of host materials and/or an organic electroluminescent compound according to the present disclosure, and the device property thereof will be explained in order to understand the present disclosure in detail.

### [Device Examples 1 to 4] Preparation of Green Light-Emitting OLEDs Deposited with a Plurality of Host Materials According to the Present Disclosure

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates and Compound HI-1 was deposited in a doping amount of 3 wt % based on the total amount of compounds HI-1 and HT-1 to form a hole injection layer having a thickness of 10 n. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: each of the first host compound and the second host compound described in the following Table 1 were introduced into two cells of the vacuum vapor deposition apparatus as hosts, respectively, and Compound D-130 was introduced into another cell as a dopant. The two host materials were evaporated at a rate of 1:2 (the first host: the second host) and the dopant material was evaporated at a different rate, simultaneously, and was deposited in a doping amount of 10 wt % based on the total amount of the hosts and dopant to form a light-emitting layer having a thickness of 40 nm on the second hole transport layer. Next, compounds ETL-1 and EIL-1 as electron transport materials were deposited at a weight ratio of 40:60 to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Comparative Example 1] Preparation of an OLED Comprising the Conventional Compound as a Host

An OLED was manufactured in the same manner as in Device Example 1, except that Compound C-ref as the first host compound was used as the host of the light-emitting layer.

The driving voltage and the luminous color at a luminance of 1,000 nits and the time taken for luminance to decrease from 100% to 95% at a luminance of 20,000 nits (lifespan: T95) of the OLEDs of Device Examples 1 to 4 and Comparative Example 1 produced as described above, are measured, and the results thereof are shown in the following Table 1.

TABLE 1

| | | | Driving | | |
|---|---|---|---|---|---|
| | First | Second | Voltage | Luminous | Lifespan |
| | Host | Host | (V) | Color | (T95, hr) |
| Device | C-1 | H2-147 | 3.4 | Green | 91.9 |
| Example 1 | | | | | |
| Device | C-62 | H2-147 | 3.2 | Green | 208.0 |
| Example 2 | | | | | |
| Device | C-147 | H2-147 | 3.2 | Green | 131.9 |
| Example 3 | | | | | |
| Device | C-61 | H2-147 | 3.4 | Green | 63.3 |
| Example 4 | | | | | |
| Comparative | C-ref | H2-147 | 3.4 | Green | 3.1 |
| Example 1 | | | | | |

As shown in Table 1 above, it can be confirmed that the organic electroluminescent device deposited with a plurality of host materials according to the present disclosure exhibits the same or lower voltage characteristics and especially significantly improved characteristics in terms of lifespan, compared to the organic electroluminescent device including a conventional host combination.

### [Device Examples 5 to 8] Preparation of Green Light-Emitting OLEDs Deposited with a Plurality of Host Materials According to the Present Disclosure

OLEDs were manufactured in the same manner as in Device Examples 1 to 4, except that the second host compound substituted with deuterium was used.

The driving voltage and the luminous color at a luminance of 1,000 nits and the time taken for luminance to decrease from 100% to 95% at a luminance of 20,000 nits (lifespan: T95) of the OLED devices of Device Examples 5 to 8 produced as described above, are measured, and the results thereof are shown in the following Table 2.

TABLE 2

| | First Host | Second Host | Luminous | Lifespan |
|---|---|---|---|---|
| | Compound | Compound | Color | (T95, hr) |
| Device Example 5 | C-1 | H2-2-D22 | Green | 101.2 |
| Device Example 6 | C-62 | H2-2-D22 | Green | 252.0 |
| Device Example 7 | C-147 | H2-2-D22 | Green | 217.7 |
| Device Example 8 | C-61 | H2-2-D22 | Green | 67.8 |

As shown in Table 2 above, it can be confirmed that the lifespan is further improved by using the second host compound substituted with deuterium according to the present disclosure, compared to the device characteristics shown in Table 1 above. This can be expected to be because the stability of the deuterated organic electroluminescent compounds is increased by lowering the zero point vibration energy of the deuterated organic electroluminescent compounds and then increasing the bond dissociation energy (BDE) of the deuterated organic electroluminescent compounds.

### [Device Examples 9 and 10] Preparation of Green Light-Emitting OLEDs Deposited with a Single Host Material According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates and Compound HI-1 was deposited in a doping amount of 3 wt % based on the total amount of compounds HI-1 and HT-1 to form a hole injection layer having a thickness of 10 nm. Next, Compound HT-1 was deposited as a first hole transport layer, having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: the host compound described in the following Table 3 was introduced into a cell of the vacuum vapor deposition apparatus as a host, and Compound D-130 was introduced into another cell as a dopant. The dopant material was evaporated at a different rate, simultaneously, and was deposited in a doping amount of 10 wt % based on the total amount of the host and dopant to form a light-emitting layer having a thickness of 40 nm on the second hole transport layer. Next, compounds ETL-1 and EIL-1 as electron transport materials were deposited at a weight ratio of 40:60 to form an electron transport layer having a thickness of 35 nm on the light-emitting layer. After depositing Compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Comparative Example 2] Preparation of an OLED Comprising the Conventional Compound as a Host

An OLED was manufactured in the same manner as in Device Example 9, except that compound C-61 was used as the host of the light-emitting layer.

The driving voltage and the luminous color at a luminance of 1,000 nits and the time taken for luminance to decrease from 100% to 95% at a luminance of 20,000 nits (lifespan: T95) of the OLED devices of Device Examples 9 and 10 and Comparative Example 2 produced as described above, are measured, and the results thereof are shown in the following Table 3.

TABLE 3

| | | Driving Voltage | Luminous | Lifespan |
|---|---|---|---|---|
| | Host | (V) | Color | (T95, hr) |
| Device | C-62 | 2.7 | Green | 16.5 |
| Example 9 | | | | |
| Device | C-147 | 2.7 | Green | 8.0 |
| Example 10 | | | | |
| Comparative | C-61 | 2.9 | Green | 5.3 |
| Example 2 | | | | |

As shown in Table 3 above, it can be confirmed that the organic electroluminescent device containing the organic electroluminescent compound according to the present disclosure as a host material shows excellent lifespan characteristics, compared to the organic electroluminescent device containing the conventional host material.

The compounds used in Device Examples and Comparative Examples above are specifically shown in the following Table 4.

TABLE 4

| | |
|---|---|
| Hole Injection Layer/Hole Transport Layer | 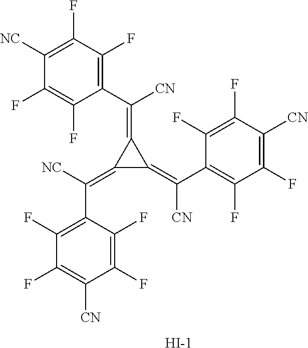 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 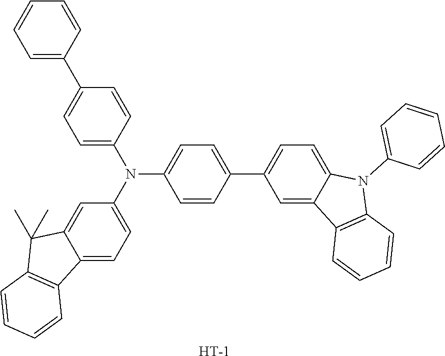 |
| | 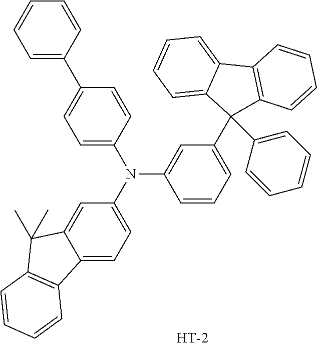 |
| Light-Emitting Layer | 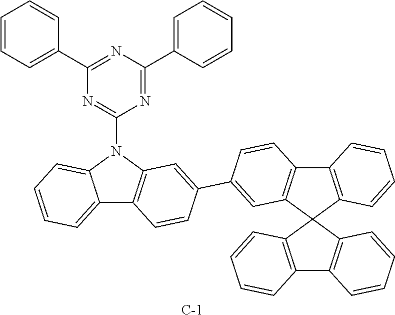 |
| | 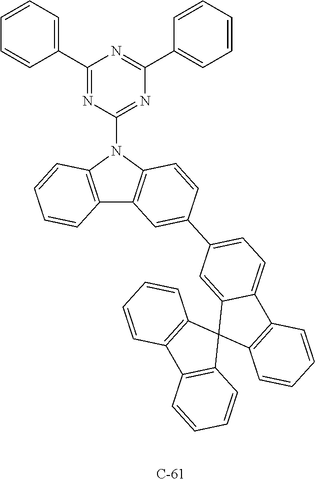 |
| | 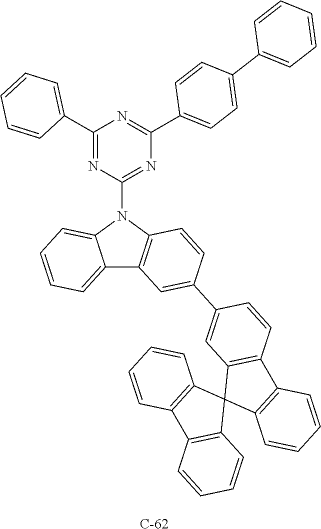 |
| | 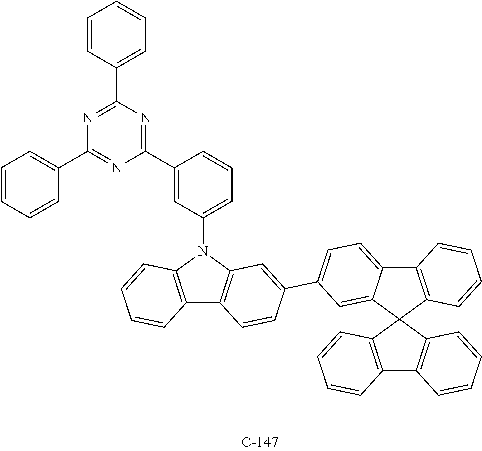 [SMILES: c1ccc(-c2nc(-c3ccccc3)nc(-c3cccc(-n4c5ccccc5c5ccc(-c6ccc7c(c6)C6(c8ccccc8-c8ccccc86)c6ccccc6-7)cc54)c3)n2)cc1] |
| | 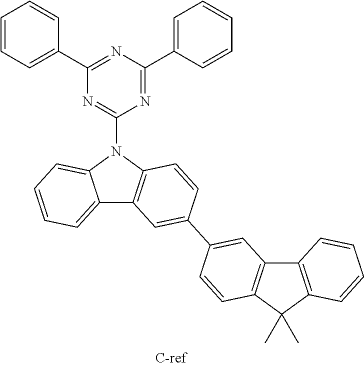 [SMILES: CC1(C)c2ccccc2-c2cc(-c3ccc4c(c3)c3ccccc3n4-c3nc(-c4ccccc4)nc(-c4ccccc4)n3)ccc21] |
| | 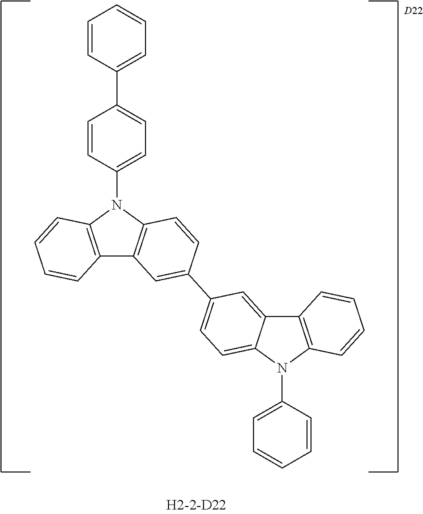 |
| | 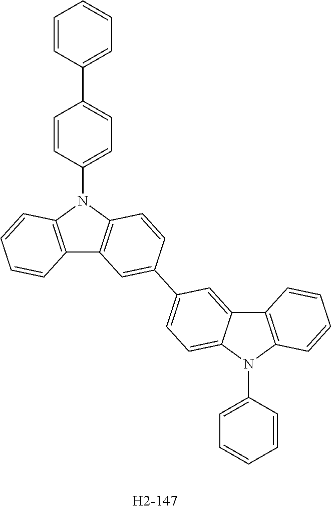 [SMILES: c1ccc(-c2ccc(-n3c4ccccc4c4cc(-c5ccc6c(c5)c5ccccc5n6-c5ccccc5)ccc43)cc2)cc1] |
| | 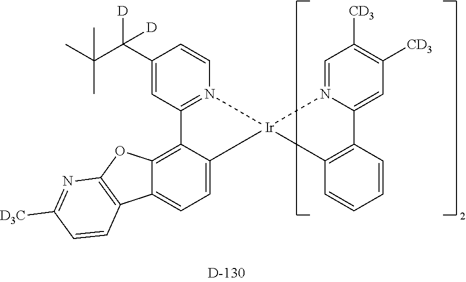 |
| Electron Transport Layer/ Electron Injection Layer | 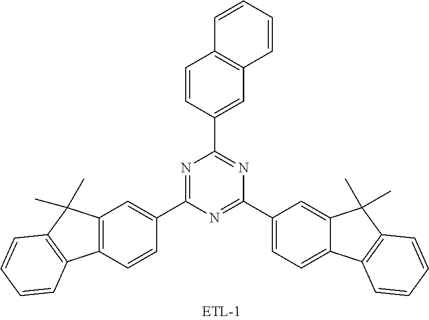 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 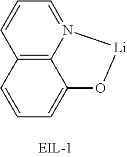 |

1. A plurality of host materials comprising at least one first host compound and at least one second host compound, wherein the first host compound is represented by the following Formula 1, the second host compound is represented by the following Formula 2, and at least one of the first host compound and the second host compound includes at least one deuterium. 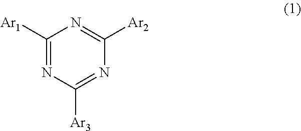
wherein,
Ar₁ to Ar₃ each independently represent, a substituted or unsubstituted (C6-C30)aryl or a substituted or unsubstituted (3- to 30-membered)heteroaryl; provided that at least one of Ar₁ to Ar₃ is a carbazole group represented by the following Formula 1-1; 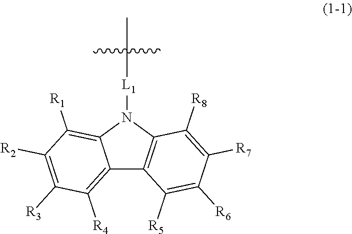
wherein,
L₁ represents a single bond or (C6-C30)arylene unsubstituted or substituted with deuterium;
R₁ to R₈ each independently represent, hydrogen, deuterium, or (C6-C30)aryl unsubstituted or substituted with deuterium, (C1-C30)alkyl, (C6-C30)aryl, or a combination thereof; provided that at least one of R₁ to R₄ is represented by the following formulas 1-11 or 1-12; 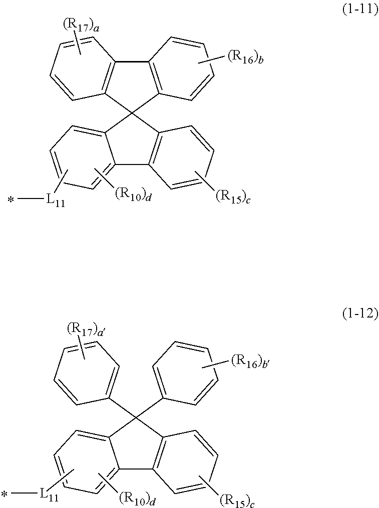
wherein,
L₁₁ represents a single bond or (C6-C30)arylene unsubstituted or substituted with deuterium;
R₁₀ and R₁₅ to R₁₇ each independently represent, hydrogen, deuterium, or (C6-C30)aryl unsubstituted or substituted with deuterium;
a to c each independently represent, an integer of 1 to 4; d is an integer of 1 to 3; a′ and b′ each independently represent, an integer of 1 to 5; when a to d, a′, and b′ are 2 or more, each of R₁₀ and each of R₁₅ to each of R₁ may be the same or different;
* represents a linking site with R₁ to R₄; 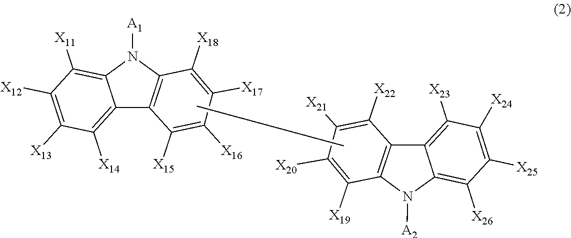
wherein,
A₁ and A₂ each independently represent, (C6-C30)aryl unsubstituted or substituted with deuterium, a substituted or unsubstituted dibenzofuranyl, a substituted or unsubstituted dibenzothiophenyl, or a substituted or unsubstituted carbazolyl;
X₁₁ to X₂₆ each independently represent hydrogen, deuterium, a substituted or unsubstituted (C6-C30)aryl, or a substituted or unsubstituted (3- to 30-membered)heteroaryl; or may be linked to the adjacent substituents to form a ring(s); and
any one of X₁₅ to X₁₈ is linked to any one of X₁₉ to X₂₂.

2. The plurality of host materials according to claim 1, wherein Ar₁ to Ar₃ in Formula 1 each independently represent, phenyl unsubstituted or substituted with deuterium, biphenyl unsubstituted or substituted with deuterium, terphenyl unsubstituted or substituted with deuterium, quarterphenyl unsubstituted or substituted with deuterium, naphthyl unsubstituted or substituted with deuterium, phenylnaphthyl unsubstituted or substituted with deuterium, naphthylphenyl unsubstituted or substituted with deuterium, dibenzofuranyl unsubstituted or substituted with deuterium, dibenzothiophenyl unsubstituted or substituted with deuterium, carbazolyl unsubstituted or substituted with deuterium, or a combination thereof.

3. The plurality of host materials according to claim 1, wherein R₁ to R₈ in Formula 1-1 each independently represent, hydrogen, deuterium, phenyl unsubstituted or substituted with deuterium, biphenyl unsubstituted or substituted with deuterium, terphenyl unsubstituted or substituted with deuterium, quarterphenyl unsubstituted or substituted with deuterium, naphthyl unsubstituted or substituted with deuterium, phenylnaphthyl unsubstituted or substituted with deuterium, naphthylphenyl unsubstituted or substituted with deuterium, or a combination thereof.

4. The plurality of host materials according to claim 1, wherein R₂ in Formula 1-1 is represented by the following formulas 1-11-1 or 1-12-1. 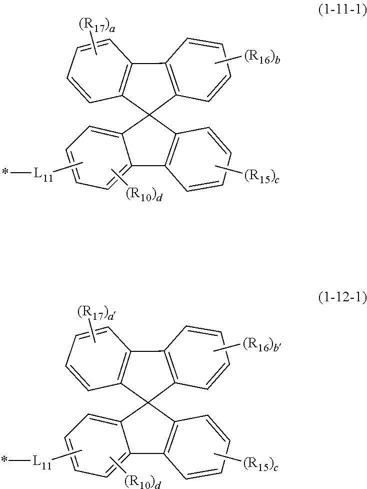
wherein,
L₁₁, R₁₀, R₁₅ to R₁₇, a to d, a′, and b′ are as defined in claim 1.

5. The plurality of host materials according to claim 1, wherein R₃ in Formula 1-1 is represented by the following formulas 1-11-1 or 1-12-1. 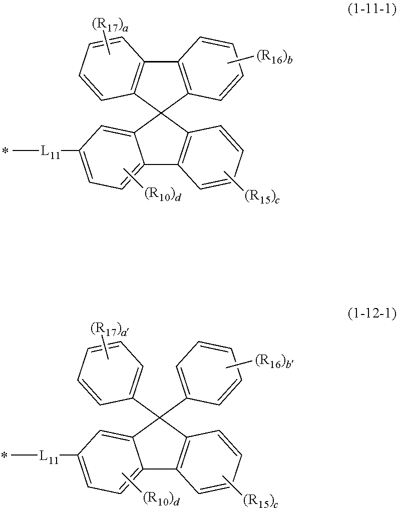
wherein,
L₁₁, R₁₀, R₁₅ to R₁₇, a to d, a′, and b′ are as defined in claim 1.

6. The plurality of host materials according to claim 1, wherein at least one of L₁ and R₁ to R₈ in Formula 1-1 includes deuterium.

7. The plurality of host materials according to claim 1, wherein at least one of X₁₁, X₁₈, X₁₉ and X₂₆ in Formula 2 is deuterium.

8. The plurality of host materials according to claim 1, wherein the deuterium substitution rate in Formula 2 is 40% to 100%.

9. The plurality of host materials according to claim 1, wherein the deuterium substitution rate of X₁₁ to X₂₆ in Formula 2 is 25% to 100%.

10. The plurality of host materials according to claim 1, wherein the Formula 2 is represented by any one of the following formulas 2-1 to 2-8: 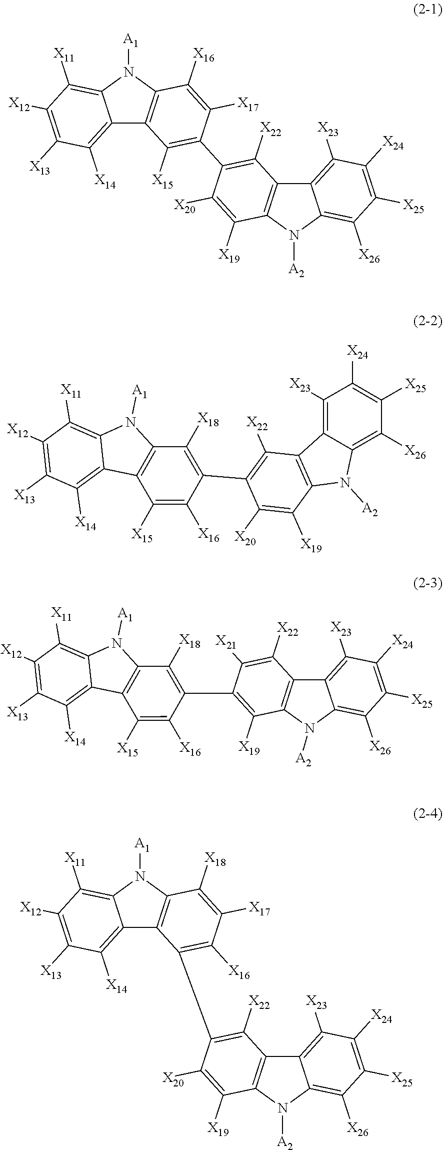 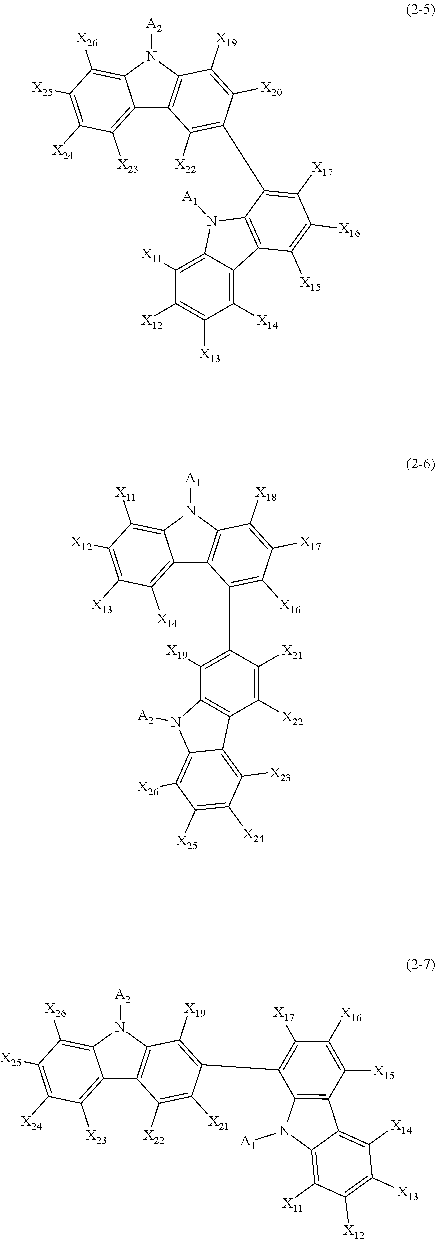 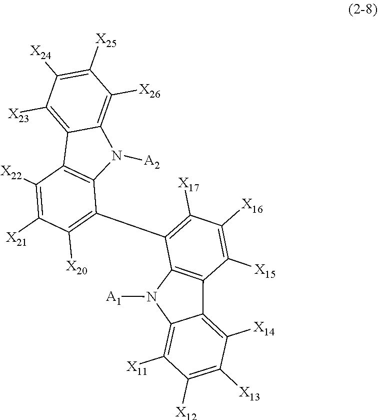
wherein,
A₁, A₂ and X₁₁ to X₂₆ are as defined in claim 1.

11. The plurality of host materials according to claim 1, wherein A₁ and A₂ in Formula 2 each independently represent, phenyl unsubstituted or substituted with deuterium, biphenyl unsubstituted or substituted with deuterium, terphenyl unsubstituted or substituted with deuterium, naphthyl unsubstituted or substituted with deuterium, fluorenyl unsubstituted or substituted with deuterium, benzofluorenyl unsubstituted or substituted with deuterium, triphenylenyl unsubstituted or substituted with deuterium, fluoranthenyl unsubstituted or substituted with deuterium, phenanthrenyl unsubstituted or substituted with deuterium, dibenzofuranyl unsubstituted or substituted with deuterium, carbazolyl unsubstituted or substituted with deuterium, dibenzothiophenyl unsubstituted or substituted with deuterium, or a combination thereof.

12. The plurality of host materials according to claim 1, wherein the first host compound represented by the Formula 1 is selected from the following compounds: 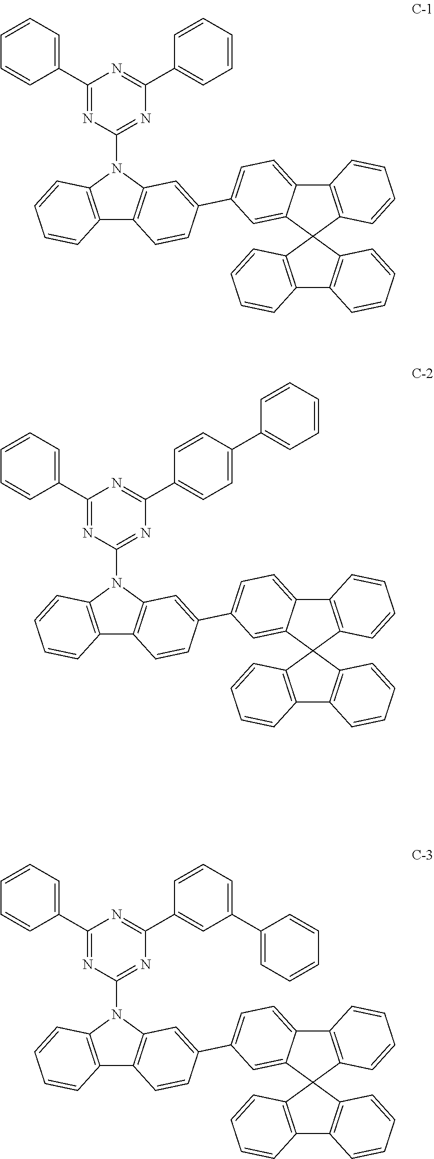 [SMILES: c1ccc(-c2nc(-c3ccccc3)nc(-n3c4ccccc4c4ccc(-c5ccc6c(c5)C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1] 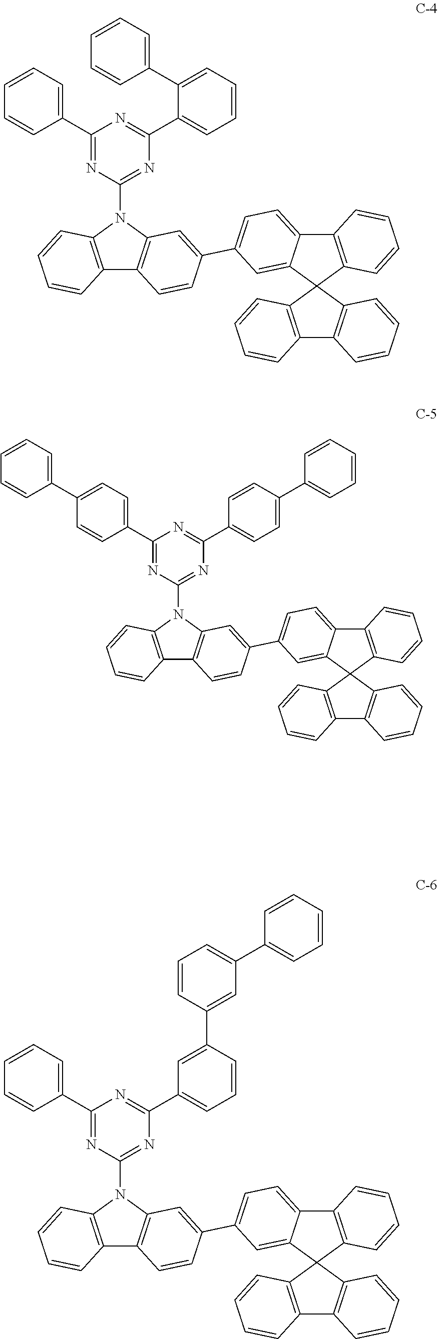 [SMILES: c1ccc(-c2nc(-c3ccccc3-c3ccccc3)nc(-n3c4ccccc4c4ccc(-c5ccc6c(c5)C5(c7ccccc7-c7ccccc75)c5ccccc5-6)cc43)n2)cc1 ; c1ccc(-c2ccc(-c3nc(-c4ccc(-c5ccccc5)cc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)c2)cc1] 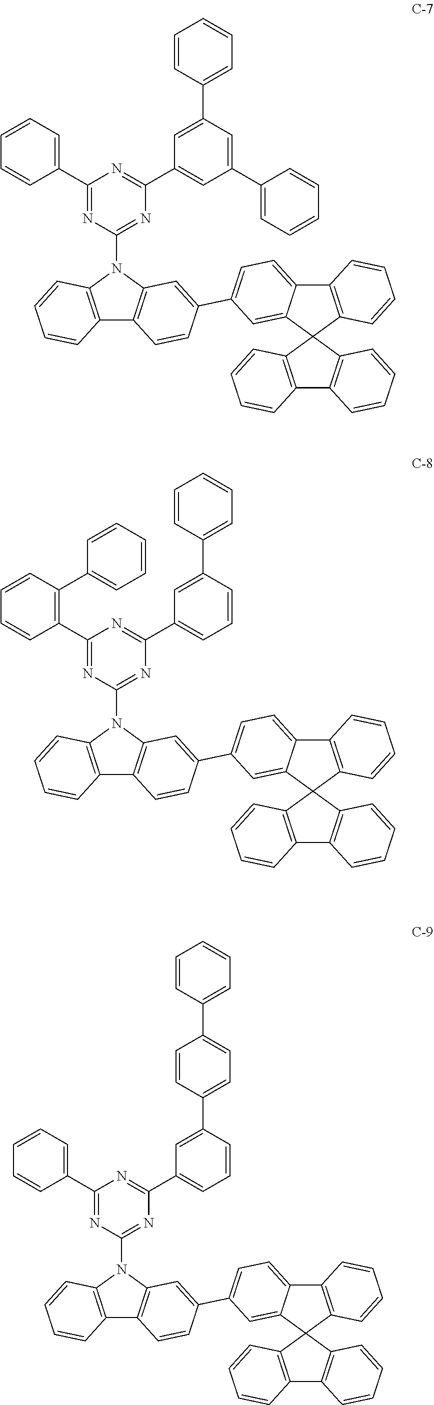 [SMILES: c1ccc(-c2cc(-c3ccccc3)cc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2cccc(-c3nc(-c4ccccc4-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2ccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)cc2)cc1] 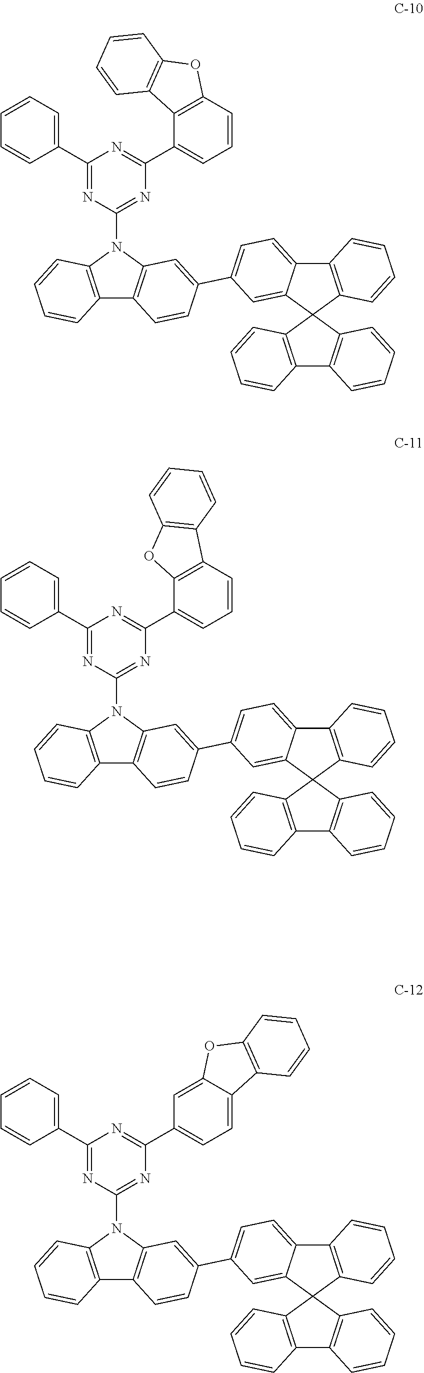 [SMILES: c1ccc(-c2nc(-c3cccc4oc5ccccc5c34)nc(-n3c4ccccc4c4ccc(-c5ccc6c(c5)C5(c7ccccc7-c7ccccc75)c5ccccc5-6)cc43)n2)cc1 ; c1ccc(-c2nc(-c3cccc4c3oc3ccccc34)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccc4c(c3)oc3ccccc34)nc(-n3c4ccccc4c4ccc(-c5ccc6c(c5)C5(c7ccccc7-c7ccccc75)c5ccccc5-6)cc43)n2)cc1] 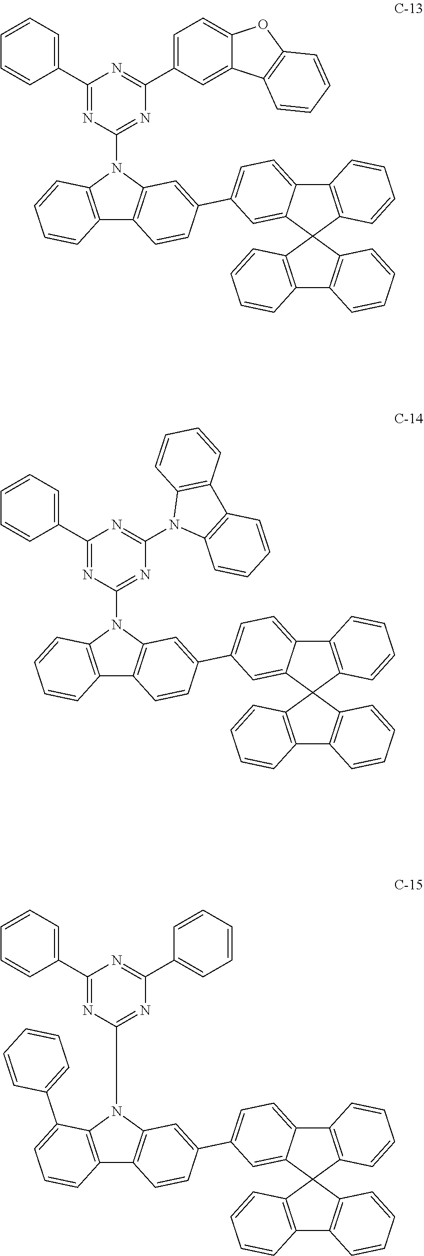 [SMILES: c1ccc(-c2nc(-c3ccc4oc5ccccc5c4c3)nc(-n3c4ccccc4c4ccc(-c5ccc6c(c5)C5(c7ccccc7-c7ccccc75)c5ccccc5-6)cc43)n2)cc1 ; c1ccc(-c2nc(-n3c4ccccc4c4ccccc43)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccccc3)nc(-n3c4cc(-c5ccc6c(c5)C5(c7ccccc7-c7ccccc75)c5ccccc5-6)ccc4c4cccc(-c5ccccc5)c43)n2)cc1] 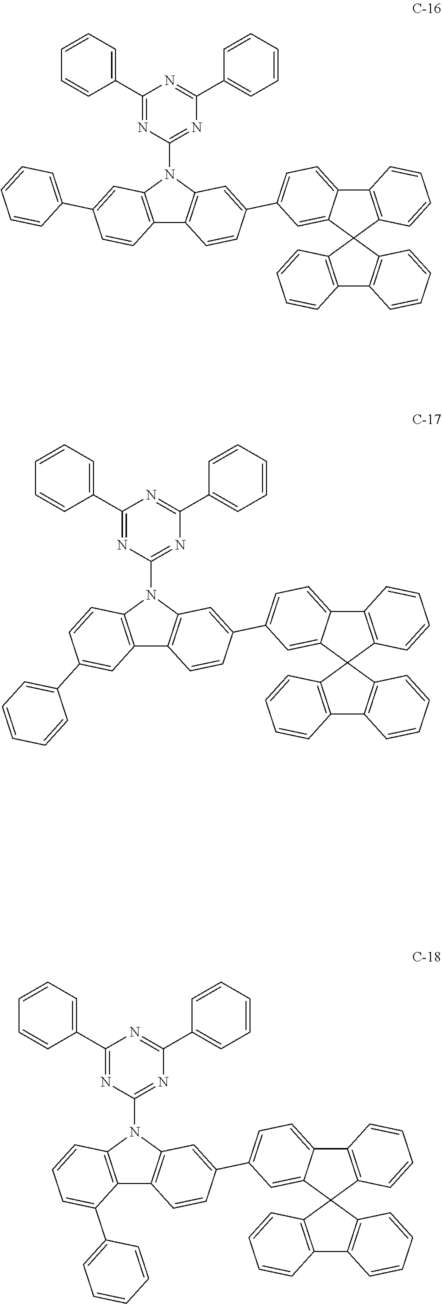 [SMILES: c1ccc(-c2ccc3c4ccc(-c5ccc6c(c5)C5(c7ccccc7-c7ccccc75)c5ccccc5-6)cc4n(-c4nc(-c5ccccc5)nc(-c5ccccc5)n4)c3c2)cc1 ; c1ccc(-c2ccc3c(c2)c2ccc(-c4ccc5c(c4)C4(c6ccccc6-c6ccccc64)c4ccccc4-5)cc2n3-c2nc(-c3ccccc3)nc(-c3ccccc3)n2)cc1 ; c1ccc(-c2nc(-c3ccccc3)nc(-n3c4cc(-c5ccc6c(c5)C5(c7ccccc7-c7ccccc75)c5ccccc5-6)ccc4c4c(-c5ccccc5)cccc43)n2)cc1] 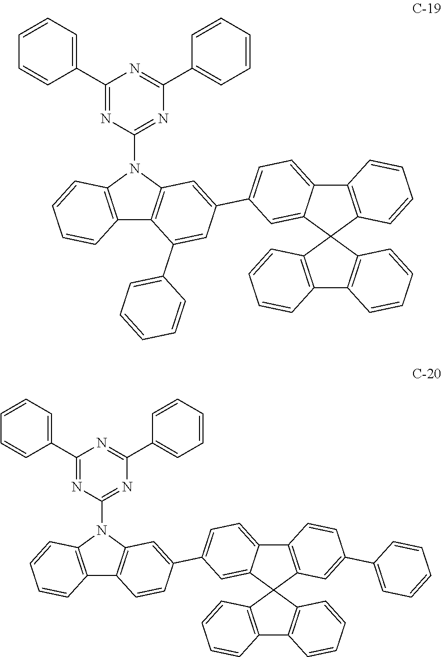 [SMILES: c1ccc(-c2nc(-c3ccccc3)nc(-n3c4ccccc4c4c(-c5ccccc5)cc(-c5ccc6c(c5)C5(c7ccccc7-c7ccccc75)c5ccccc5-6)cc43)n2)cc1 ; c1ccc(-c2ccc3c(c2)C2(c4ccccc4-c4ccccc42)c2cc(-c4ccc5c6ccccc6n(-c6nc(-c7ccccc7)nc(-c7ccccc7)n6)c5c4)ccc2-3)cc1] 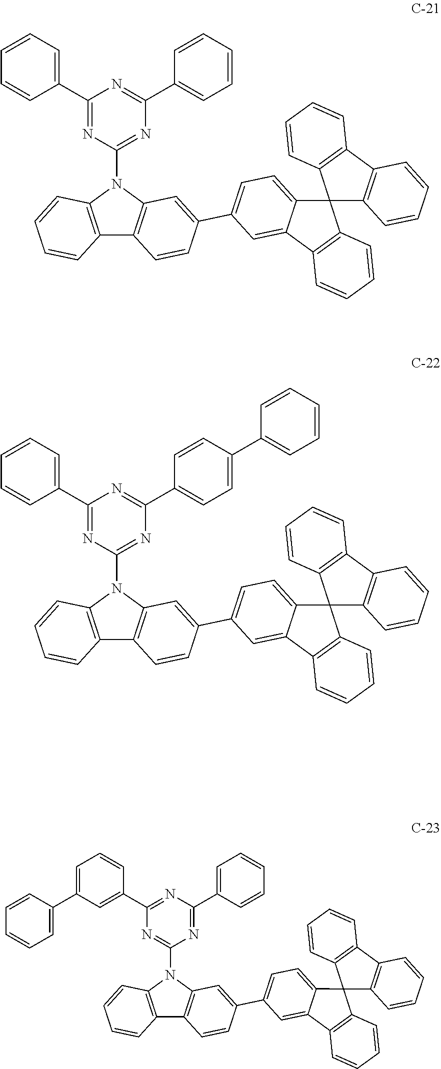 [SMILES: c1ccc(-c2nc(-c3ccccc3)nc(-n3c4ccccc4c4ccc(-c5ccc6c(c5)-c5ccccc5C65c6ccccc6-c6ccccc65)cc43)n2)cc1 ; c1ccc(-c2ccc(-c3nc(-c4ccccc4)nc(-n4c5ccccc5c5ccc(-c6ccc7c(c6)-c6ccccc6C76c7ccccc7-c7ccccc76)cc54)n3)cc2)cc1 ; c1ccc(-c2cccc(-c3nc(-c4ccccc4)nc(-n4c5ccccc5c5ccc(-c6ccc7c(c6)-c6ccccc6C76c7ccccc7-c7ccccc76)cc54)n3)c2)cc1] 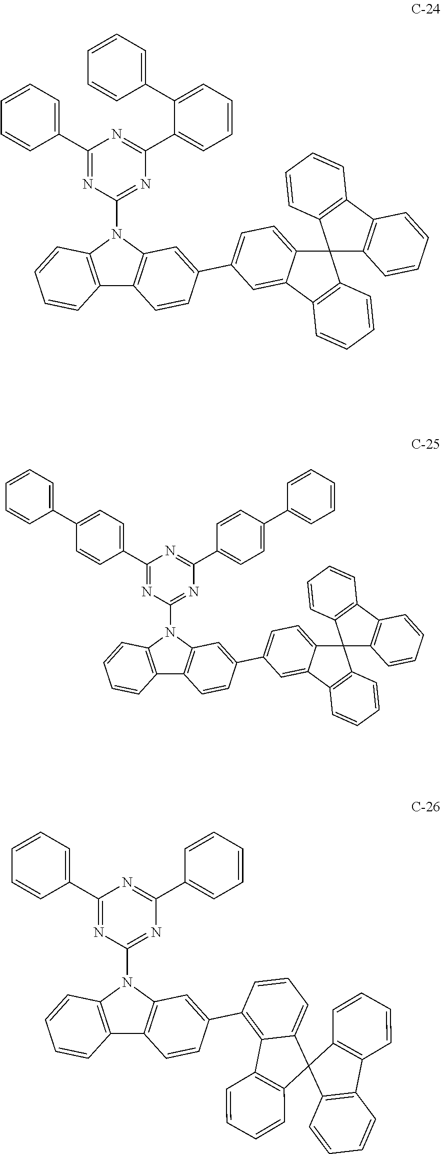 [SMILES: c1ccc(-c2nc(-c3ccccc3-c3ccccc3)nc(-n3c4ccccc4c4ccc(-c5ccc6c(c5)-c5ccccc5C65c6ccccc6-c6ccccc65)cc43)n2)cc1 ; c1ccc(-c2ccc(-c3nc(-c4ccc(-c5ccccc5)cc4)nc(-n4c5ccccc5c5ccc(-c6ccc7c(c6)-c6ccccc6C76c7ccccc7-c7ccccc76)cc54)n3)cc2)cc1 ; c1ccc(-c2nc(-c3ccccc3)nc(-n3c4ccccc4c4ccc(-c5cccc6c5-c5ccccc5C65c6ccccc6-c6ccccc65)cc43)n2)cc1] 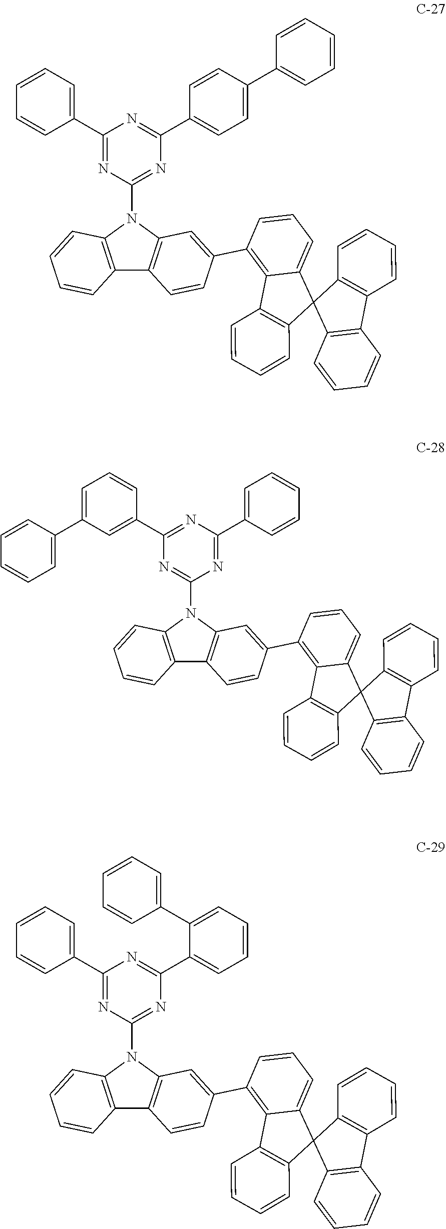 [SMILES: c1ccc(-c2ccc(-c3nc(-c4ccccc4)nc(-n4c5ccccc5c5ccc(-c6cccc7c6-c6ccccc6C76c7ccccc7-c7ccccc76)cc54)n3)cc2)cc1 ; c1ccc(-c2cccc(-c3nc(-c4ccccc4)nc(-n4c5ccccc5c5ccc(-c6cccc7c6-c6ccccc6C76c7ccccc7-c7ccccc76)cc54)n3)c2)cc1 ; c1ccc(-c2nc(-c3ccccc3-c3ccccc3)nc(-n3c4ccccc4c4ccc(-c5cccc6c5-c5ccccc5C65c6ccccc6-c6ccccc65)cc43)n2)cc1] 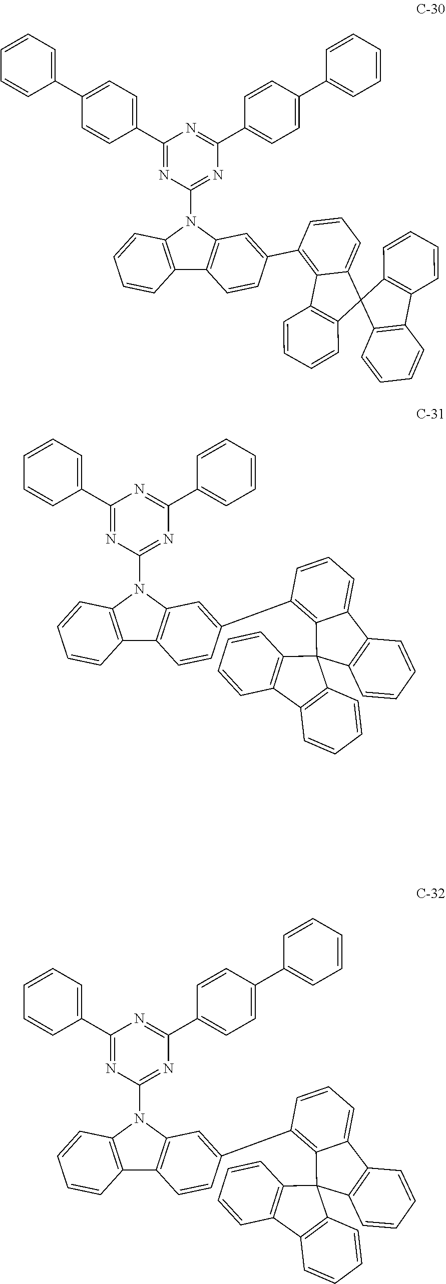 [SMILES: c1ccc(-c2ccc(-c3nc(-c4ccc(-c5ccccc5)cc4)nc(-n4c5ccccc5c5ccc(-c6cccc7c6-c6ccccc6C76c7ccccc7-c7ccccc76)cc54)n3)cc2)cc1 ; c1ccc(-c2nc(-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6cccc7c6C6(c8ccccc8-c8ccccc86)c6ccccc6-7)cc54)n3)cc2)cc1] 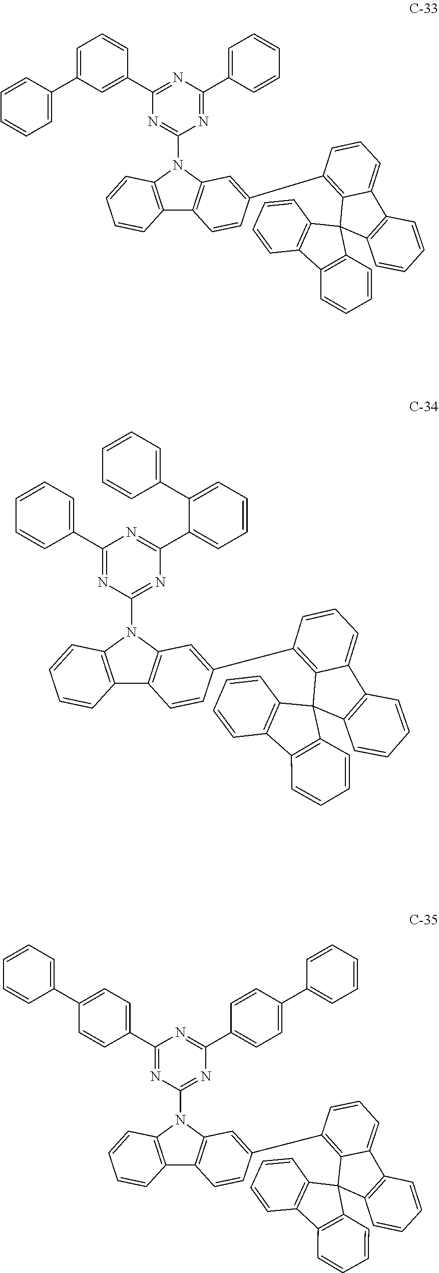 [SMILES: c1ccc(-c2cccc(-c3nc(-c4ccccc4)nc(-n4c5ccccc5c5ccc(-c6cccc7c6C6(c8ccccc8-c8ccccc86)c6ccccc6-7)cc54)n3)c2)cc1 ; c1ccc(-c2nc(-c3ccccc3-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccc(-c5ccccc5)cc4)nc(-n4c5ccccc5c5ccc(-c6cccc7c6C6(c8ccccc8-c8ccccc86)c6ccccc6-7)cc54)n3)cc2)cc1] 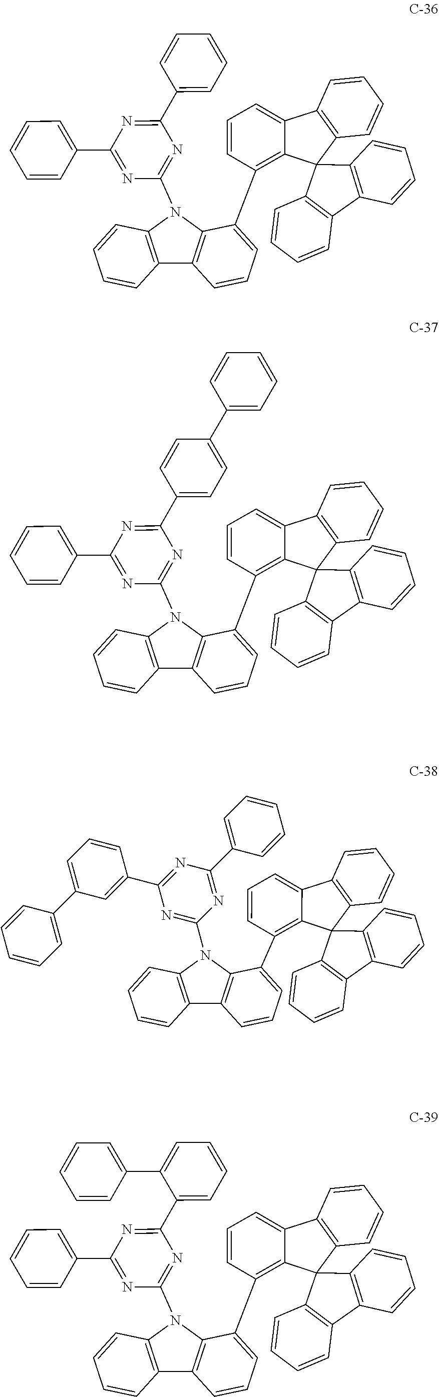 [SMILES: c1ccc(-c2nc(-c3ccccc3)nc(-n3c4ccccc4c4cccc(-c5cccc6c5C5(c7ccccc7-c7ccccc75)c5ccccc5-6)c43)n2)cc1 ; c1ccc(-c2ccc(-c3nc(-c4ccccc4)nc(-n4c5ccccc5c5cccc(-c6cccc7c6C6(c8ccccc8-c8ccccc86)c6ccccc6-7)c54)n3)cc2)cc1 ; c1ccc(-c2cccc(-c3nc(-c4ccccc4)nc(-n4c5ccccc5c5cccc(-c6cccc7c6C6(c8ccccc8-c8ccccc86)c6ccccc6-7)c54)n3)c2)cc1 ; c1ccc(-c2nc(-c3ccccc3-c3ccccc3)nc(-n3c4ccccc4c4cccc(-c5cccc6c5C5(c7ccccc7-c7ccccc75)c5ccccc5-6)c43)n2)cc1] 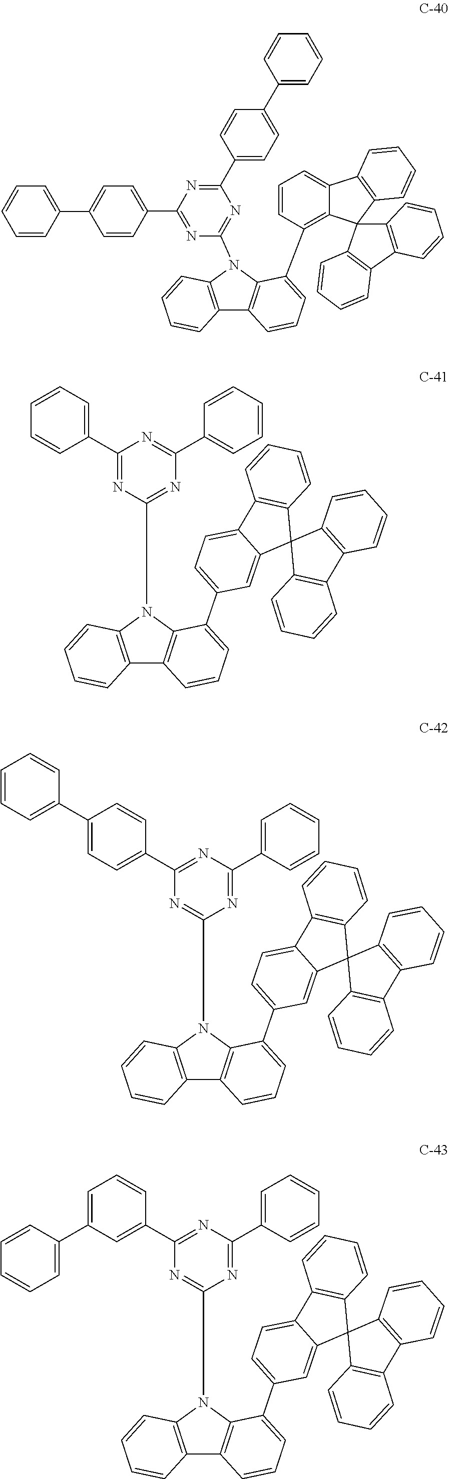 [SMILES: c1ccc(-c2ccc(-c3nc(-c4ccc(-c5ccccc5)cc4)nc(-n4c5ccccc5c5cccc(-c6cccc7c6C6(c8ccccc8-c8ccccc86)c6ccccc6-7)c54)n3)cc2)cc1 ; c1ccc(-c2nc(-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccccc4)nc(-n4c5ccccc5c5cccc(-c6ccc7c(c6)C6(c8ccccc8-c8ccccc86)c6ccccc6-7)c54)n3)cc2)cc1 ; c1ccc(-c2cccc(-c3nc(-c4ccccc4)nc(-n4c5ccccc5c5cccc(-c6ccc7c(c6)C6(c8ccccc8-c8ccccc86)c6ccccc6-7)c54)n3)c2)cc1] 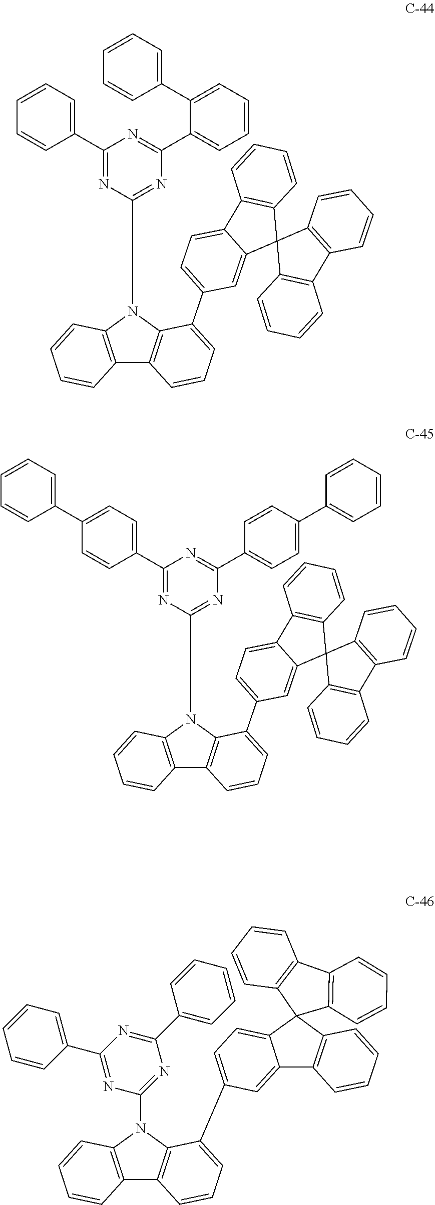 [SMILES: c1ccc(-c2nc(-c3ccccc3-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccc(-c5ccccc5)cc4)nc(-n4c5ccccc5c5cccc(-c6ccc7c(c6)C6(c8ccccc8-c8ccccc86)c6ccccc6-7)c54)n3)cc2)cc1 ; c1ccc(-c2nc(-c3ccccc3)nc(-n3c4ccccc4c4cccc(-c5ccc6c(c5)-c5ccccc5C65c6ccccc6-c6ccccc65)c43)n2)cc1] 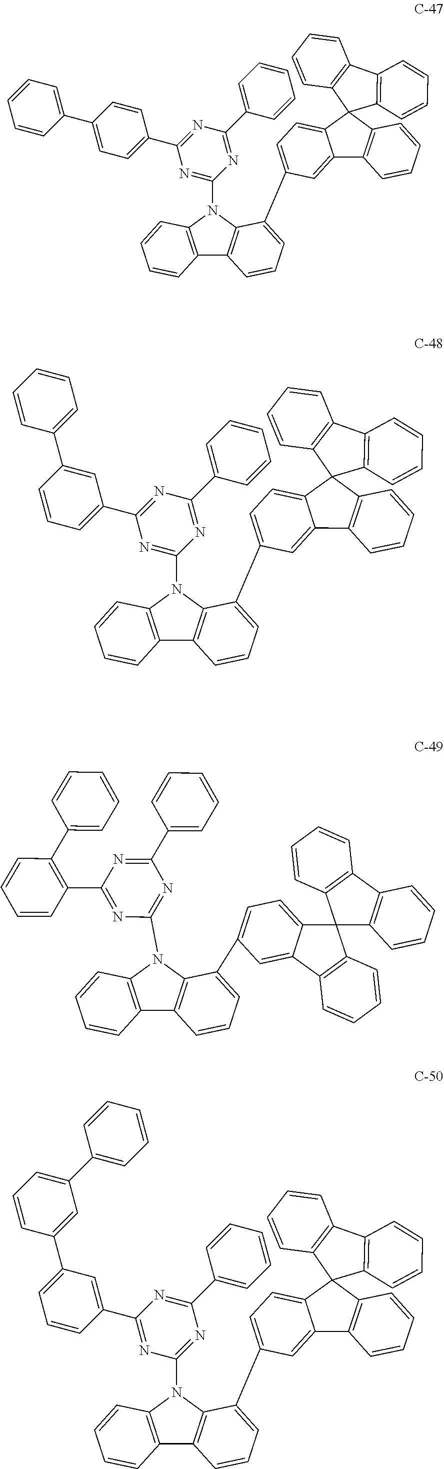 [SMILES: c1ccc(-c2ccc(-c3nc(-c4ccccc4)nc(-n4c5ccccc5c5cccc(-c6ccc7c(c6)-c6ccccc6C76c7ccccc7-c7ccccc76)c54)n3)cc2)cc1 ; c1ccc(-c2cccc(-c3nc(-c4ccccc4)nc(-n4c5ccccc5c5cccc(-c6ccc7c(c6)-c6ccccc6C76c7ccccc7-c7ccccc76)c54)n3)c2)cc1 ; c1ccc(-c2nc(-c3ccccc3-c3ccccc3)nc(-n3c4ccccc4c4cccc(-c5ccc6c(c5)-c5ccccc5C65c6ccccc6-c6ccccc65)c43)n2)cc1 ; c1ccc(-c2cccc(-c3cccc(-c4nc(-c5ccccc5)nc(-n5c6ccccc6c6cccc(-c7ccc8c(c7)-c7ccccc7C87c8ccccc8-c8ccccc87)c65)n4)c3)c2)cc1] 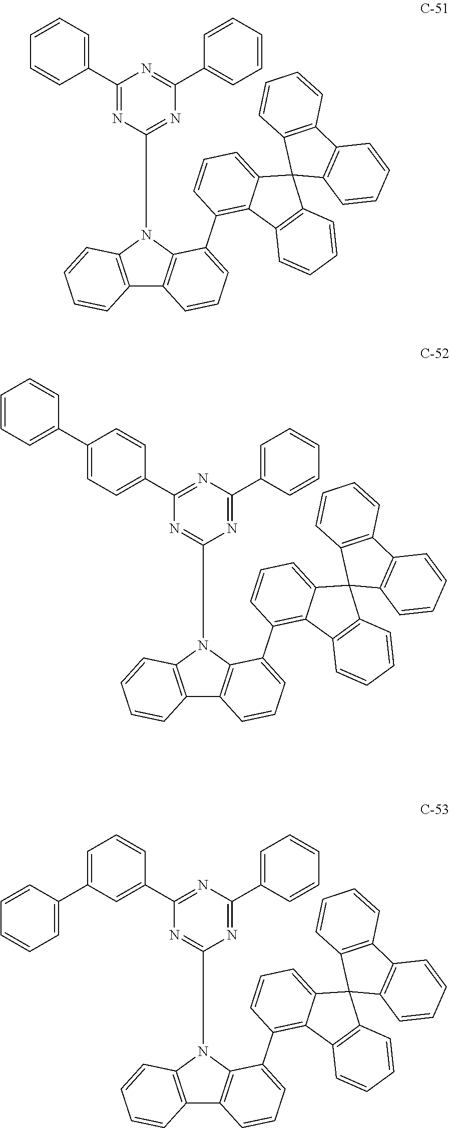 [SMILES: c1ccc(-c2nc(-c3ccccc3)nc(-n3c4ccccc4c4cccc(-c5cccc6c5-c5ccccc5C65c6ccccc6-c6ccccc65)c43)n2)cc1 ; c1ccc(-c2ccc(-c3nc(-c4ccccc4)nc(-n4c5ccccc5c5cccc(-c6cccc7c6-c6ccccc6C76c7ccccc7-c7ccccc76)c54)n3)cc2)cc1 ; c1ccc(-c2cccc(-c3nc(-c4ccccc4)nc(-n4c5ccccc5c5cccc(-c6cccc7c6-c6ccccc6C76c7ccccc7-c7ccccc76)c54)n3)c2)cc1] 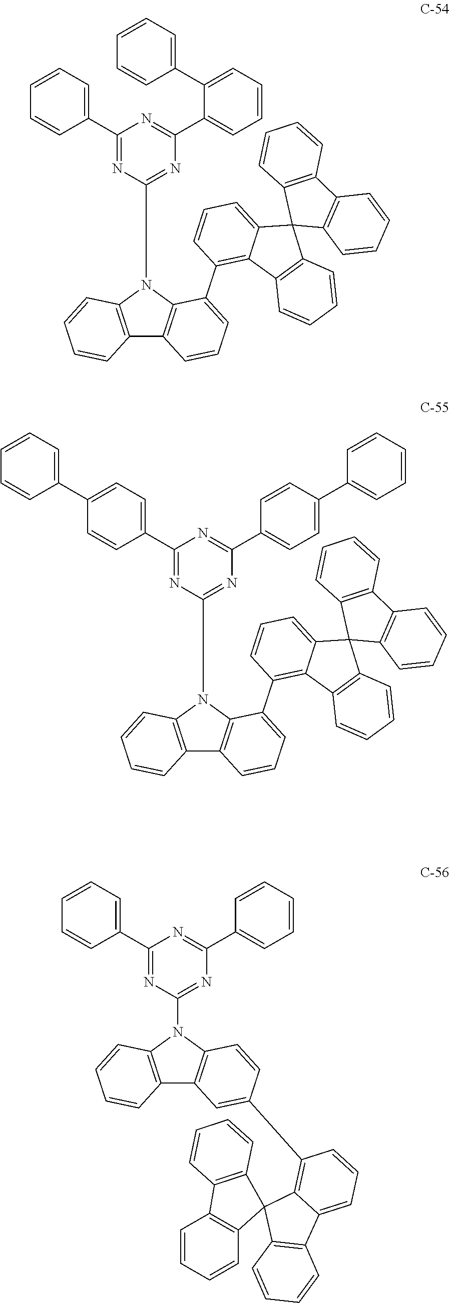 [SMILES: c1ccc(-c2nc(-c3ccccc3-c3ccccc3)nc(-n3c4ccccc4c4cccc(-c5cccc6c5-c5ccccc5C65c6ccccc6-c6ccccc65)c43)n2)cc1 ; c1ccc(-c2ccc(-c3nc(-c4ccc(-c5ccccc5)cc4)nc(-n4c5ccccc5c5cccc(-c6cccc7c6-c6ccccc6C76c7ccccc7-c7ccccc76)c54)n3)cc2)cc1 ; c1ccc(-c2nc(-c3ccccc3)nc(-n3c4ccccc4c4cc(-c5cccc6c5C5(c7ccccc7-c7ccccc75)c5ccccc5-6)ccc43)n2)cc1] 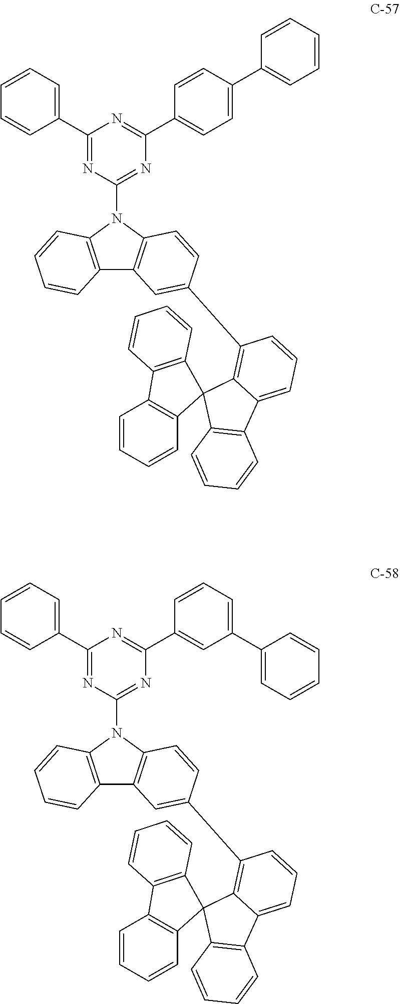 [SMILES: c1ccc(-c2ccc(-c3nc(-c4ccccc4)nc(-n4c5ccccc5c5cc(-c6cccc7c6C6(c8ccccc8-c8ccccc86)c6ccccc6-7)ccc54)n3)cc2)cc1 ; c1ccc(-c2cccc(-c3nc(-c4ccccc4)nc(-n4c5ccccc5c5cc(-c6cccc7c6C6(c8ccccc8-c8ccccc86)c6ccccc6-7)ccc54)n3)c2)cc1] 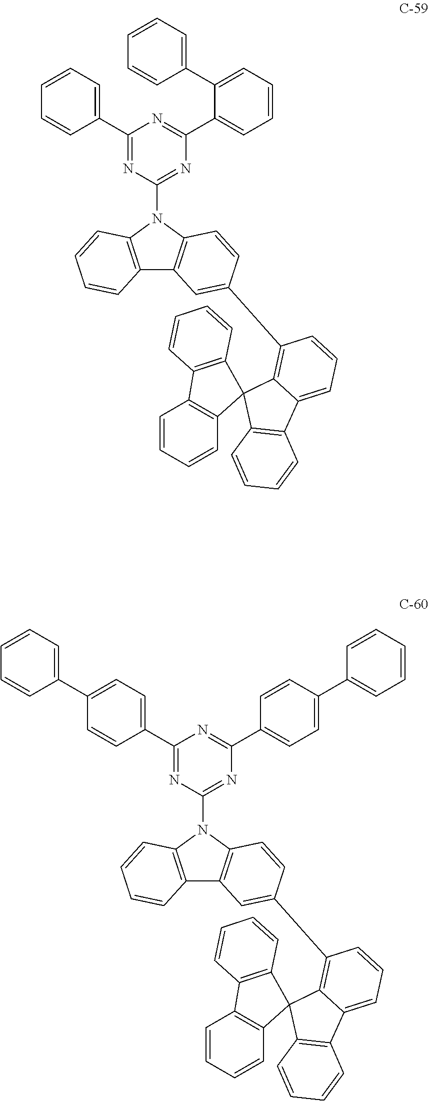 [SMILES: c1ccc(-c2nc(-c3ccccc3-c3ccccc3)nc(-n3c4ccccc4c4cc(-c5cccc6c5C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6cccc7c6C6(c8ccccc8-c8ccccc86)c6ccccc6-7)ccc54)n3)cc2)cc1] 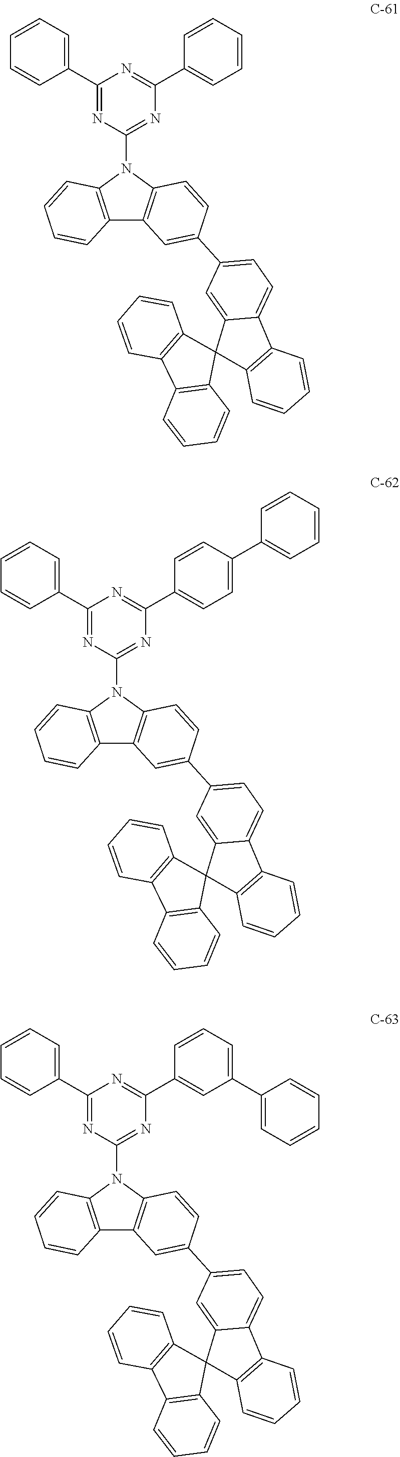 [SMILES: c1ccc(-c2nc(-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccccc4)nc(-n4c5ccccc5c5cc(-c6ccc7c(c6)C6(c8ccccc8-c8ccccc86)c6ccccc6-7)ccc54)n3)cc2)cc1 ; c1ccc(-c2cccc(-c3nc(-c4ccccc4)nc(-n4c5ccccc5c5cc(-c6ccc7c(c6)C6(c8ccccc8-c8ccccc86)c6ccccc6-7)ccc54)n3)c2)cc1] 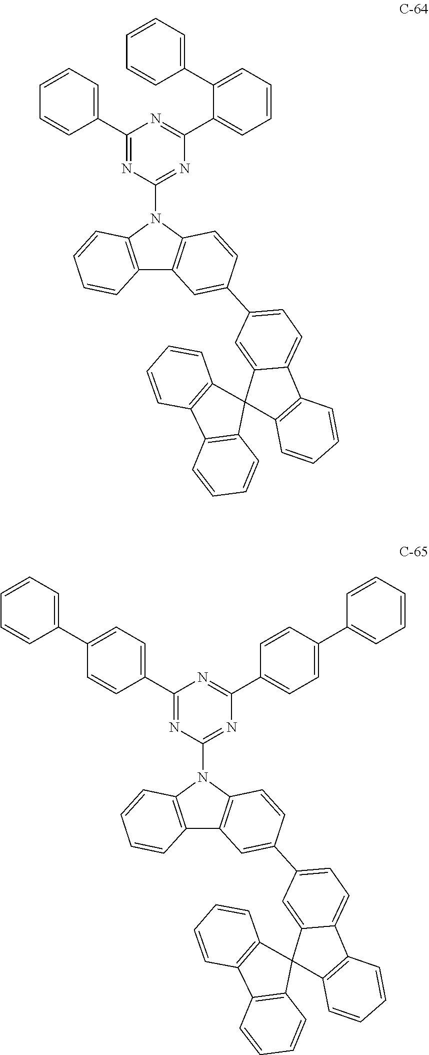 [SMILES: c1ccc(-c2nc(-c3ccccc3-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6ccc7c(c6)C6(c8ccccc8-c8ccccc86)c6ccccc6-7)ccc54)n3)cc2)cc1] 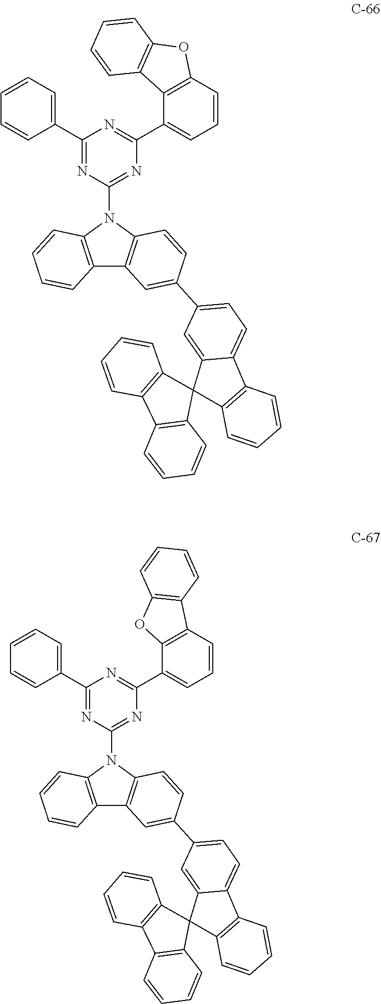 [SMILES: c1ccc(-c2nc(-c3cccc4oc5ccccc5c34)nc(-n3c4ccccc4c4cc(-c5ccc6c(c5)C5(c7ccccc7-c7ccccc75)c5ccccc5-6)ccc43)n2)cc1 ; c1ccc(-c2nc(-c3cccc4c3oc3ccccc34)nc(-n3c4ccccc4c4cc(-c5ccc6c(c5)C5(c7ccccc7-c7ccccc75)c5ccccc5-6)ccc43)n2)cc1] 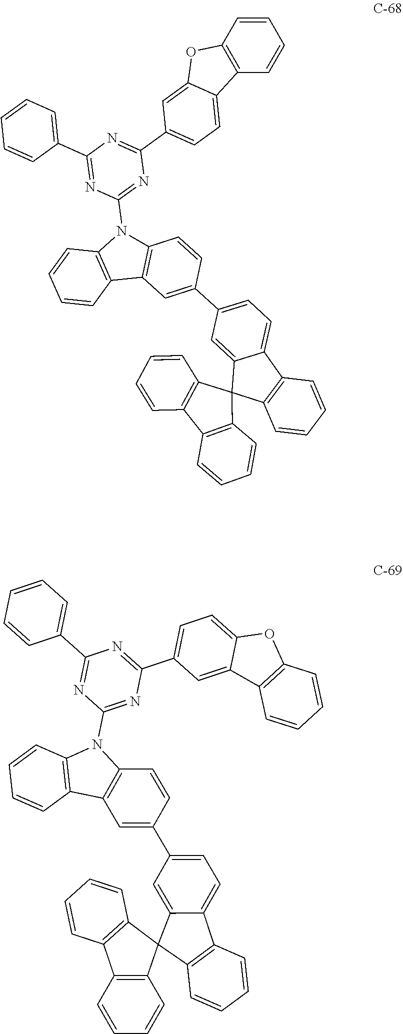 [SMILES: c1ccc(-c2nc(-c3ccc4c(c3)oc3ccccc34)nc(-n3c4ccccc4c4cc(-c5ccc6c(c5)C5(c7ccccc7-c7ccccc75)c5ccccc5-6)ccc43)n2)cc1 ; c1ccc(-c2nc(-c3ccc4oc5ccccc5c4c3)nc(-n3c4ccccc4c4cc(-c5ccc6c(c5)C5(c7ccccc7-c7ccccc75)c5ccccc5-6)ccc43)n2)cc1] 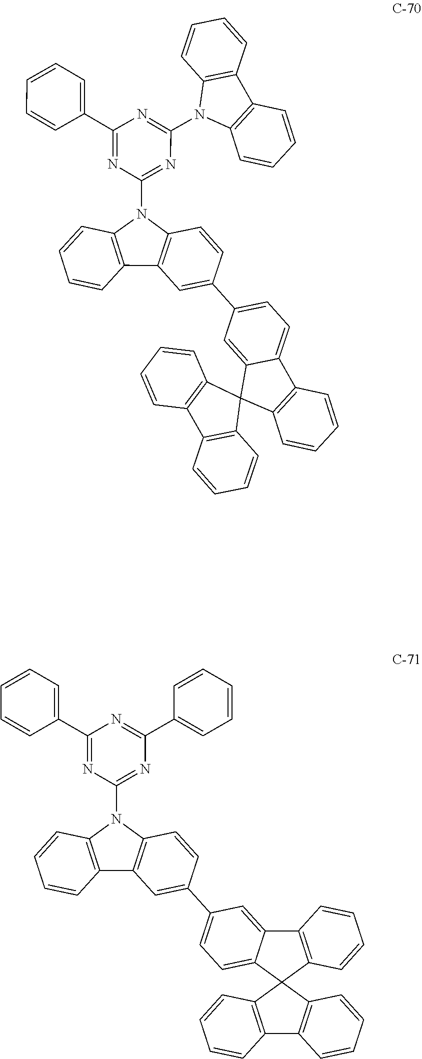 [SMILES: c1ccc(-c2nc(-n3c4ccccc4c4ccccc43)nc(-n3c4ccccc4c4cc(-c5ccc6c(c5)C5(c7ccccc7-c7ccccc75)c5ccccc5-6)ccc43)n2)cc1 ; c1ccc(-c2nc(-c3ccccc3)nc(-n3c4ccccc4c4cc(-c5ccc6c(c5)-c5ccccc5C65c6ccccc6-c6ccccc65)ccc43)n2)cc1] 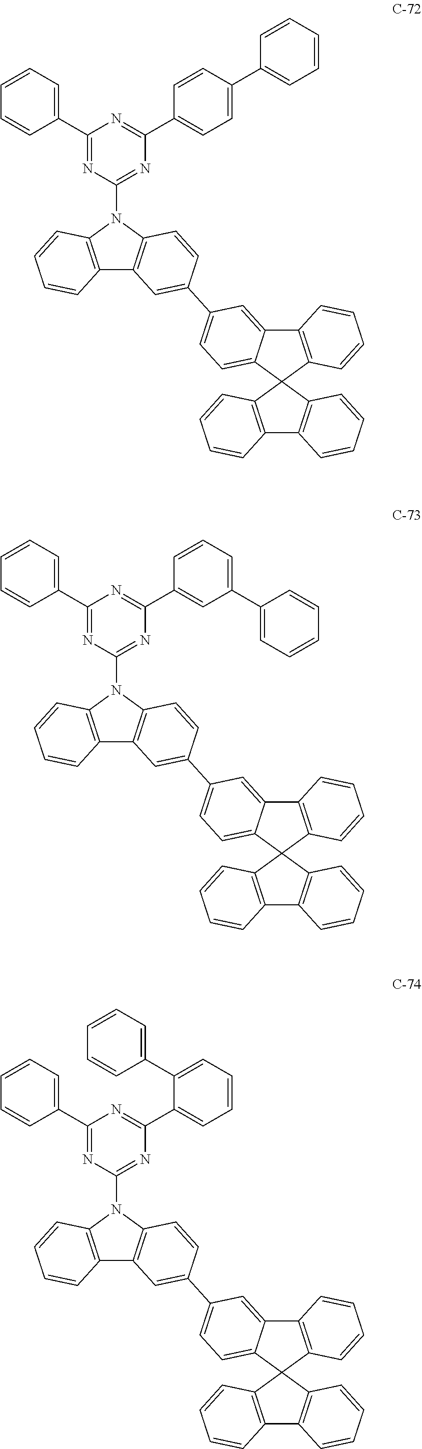 [SMILES: c1ccc(-c2ccc(-c3nc(-c4ccccc4)nc(-n4c5ccccc5c5cc(-c6ccc7c(c6)-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6ccc7c(c6)-c6ccccc6C76c7ccccc7-c7ccccc76)ccc54)n3)c2)cc1 ; c1ccc(-c2nc(-c3ccccc3-c3ccccc3)nc(-n3c4ccccc4c4cc(-c5ccc6c(c5)-c5ccccc5C65c6ccccc6-c6ccccc65)ccc43)n2)cc1] 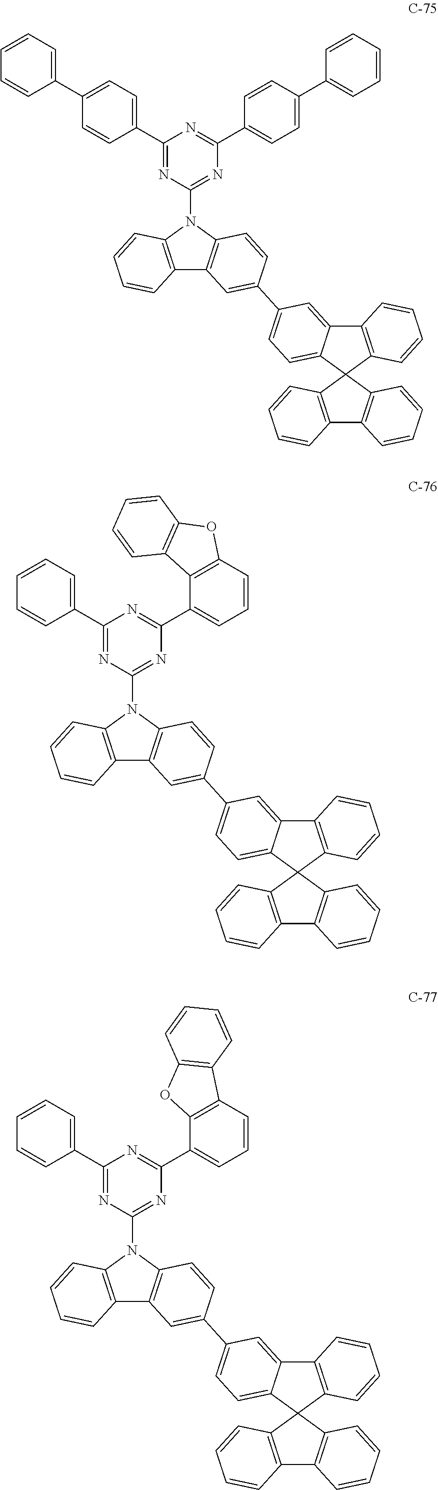 [SMILES: c1ccc(-c2ccc(-c3nc(-c4ccc(-c5ccccc5)cc4)nc(-n4c5ccccc5c5cc(-c6ccc7c(c6)-c6ccccc6C76c7ccccc7-c7ccccc76)ccc54)n3)cc2)cc1 ; c1ccc(-c2nc(-c3cccc4oc5ccccc5c34)nc(-n3c4ccccc4c4cc(-c5ccc6c(c5)-c5ccccc5C65c6ccccc6-c6ccccc65)ccc43)n2)cc1 ; c1ccc(-c2nc(-c3cccc4c3oc3ccccc34)nc(-n3c4ccccc4c4cc(-c5ccc6c(c5)-c5ccccc5C65c6ccccc6-c6ccccc65)ccc43)n2)cc1] 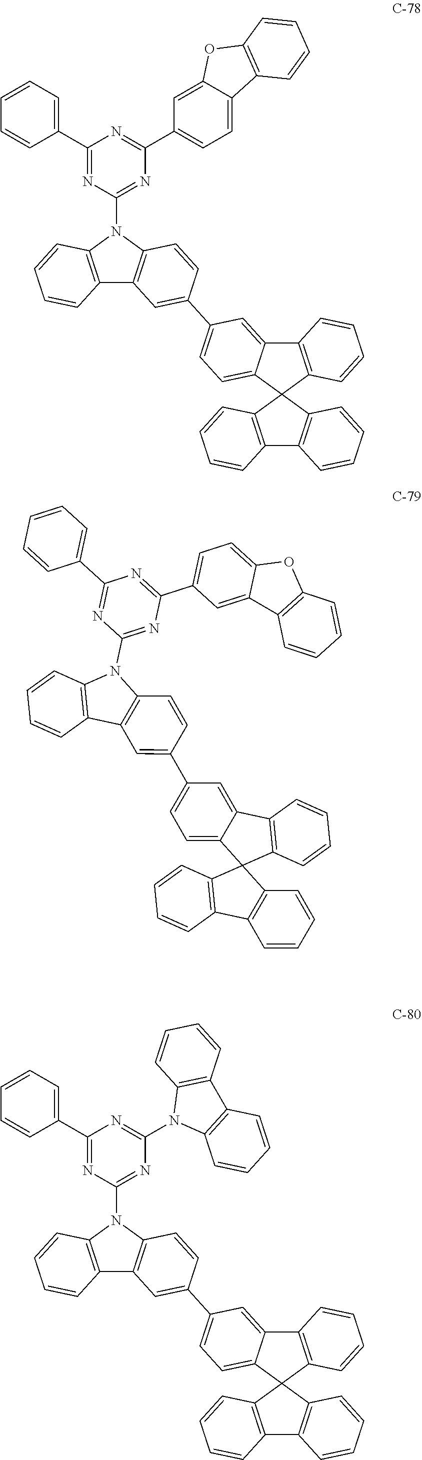 [SMILES: c1ccc(-c2nc(-c3ccc4c(c3)oc3ccccc34)nc(-n3c4ccccc4c4cc(-c5ccc6c(c5)-c5ccccc5C65c6ccccc6-c6ccccc65)ccc43)n2)cc1 ; c1ccc(-c2nc(-c3ccc4oc5ccccc5c4c3)nc(-n3c4ccccc4c4cc(-c5ccc6c(c5)-c5ccccc5C65c6ccccc6-c6ccccc65)ccc43)n2)cc1 ; c1ccc(-c2nc(-n3c4ccccc4c4ccccc43)nc(-n3c4ccccc4c4cc(-c5ccc6c(c5)-c5ccccc5C65c6ccccc6-c6ccccc65)ccc43)n2)cc1] 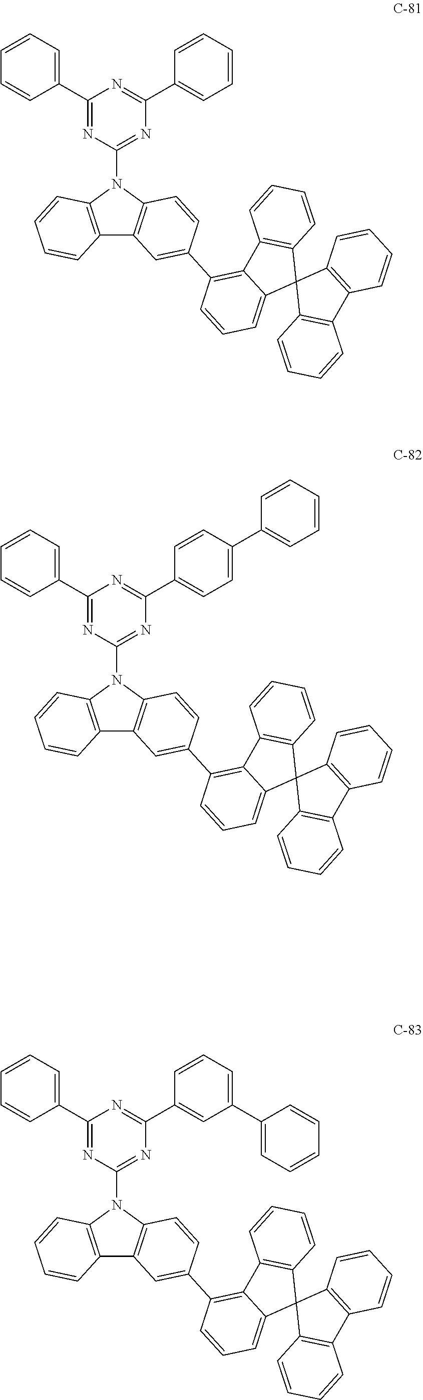 [SMILES: c1ccc(-c2nc(-c3ccccc3)nc(-n3c4ccccc4c4cc(-c5cccc6c5-c5ccccc5C65c6ccccc6-c6ccccc65)ccc43)n2)cc1 ; c1ccc(-c2ccc(-c3nc(-c4ccccc4)nc(-n4c5ccccc5c5cc(-c6cccc7c6-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6cccc7c6-c6ccccc6C76c7ccccc7-c7ccccc76)ccc54)n3)c2)cc1] 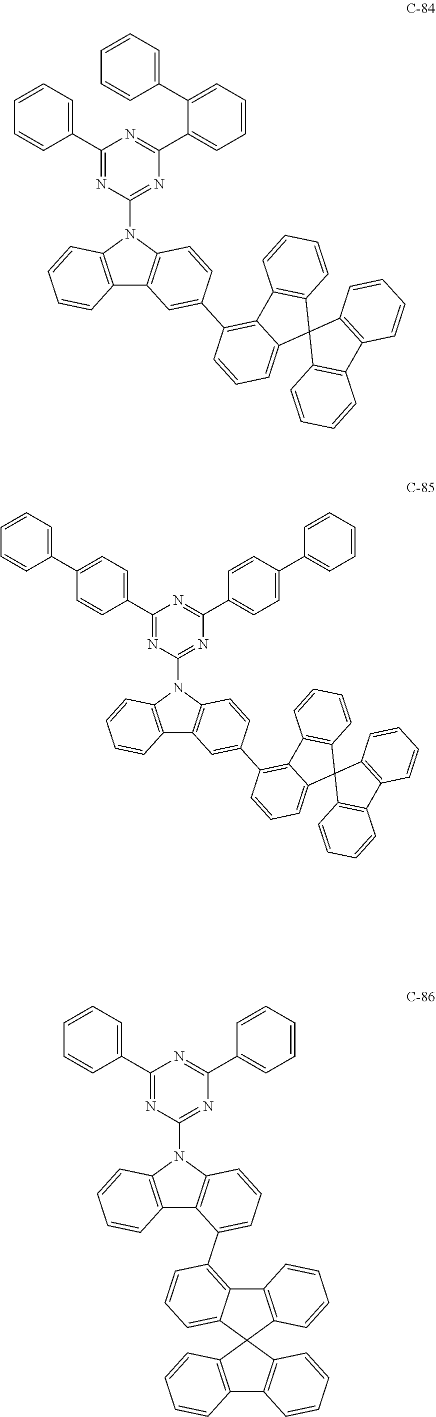 [SMILES: c1ccc(-c2nc(-c3ccccc3-c3ccccc3)nc(-n3c4ccccc4c4cc(-c5cccc6c5-c5ccccc5C65c6ccccc6-c6ccccc65)ccc43)n2)cc1 ; c1ccc(-c2ccc(-c3nc(-c4ccc(-c5ccccc5)cc4)nc(-n4c5ccccc5c5cc(-c6cccc7c6-c6ccccc6C76c7ccccc7-c7ccccc76)ccc54)n3)cc2)cc1 ; c1ccc(-c2nc(-c3ccccc3)nc(-n3c4ccccc4c4c(-c5cccc6c5-c5ccccc5C65c6ccccc6-c6ccccc65)cccc43)n2)cc1] 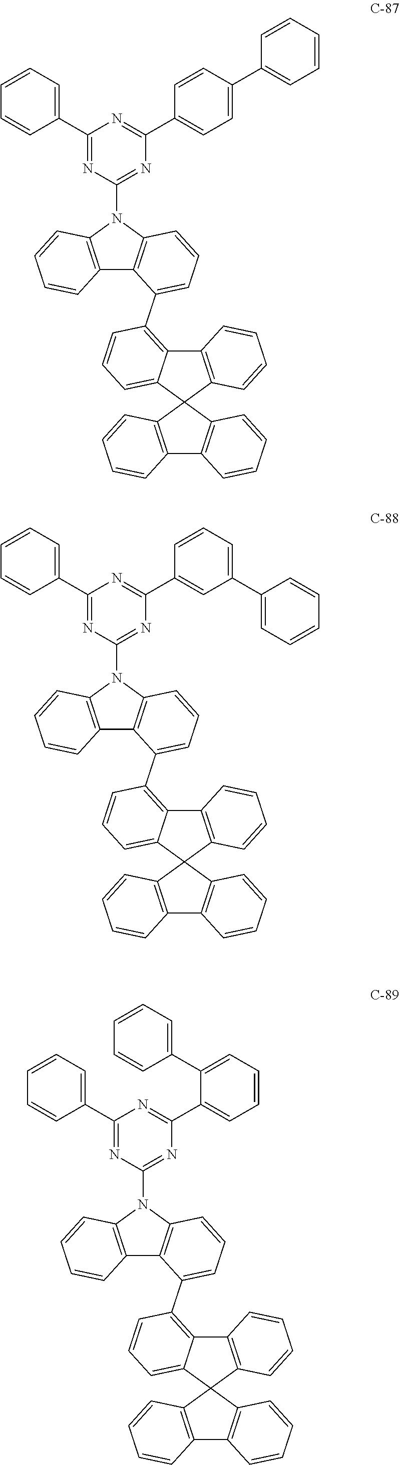 [SMILES: c1ccc(-c2ccc(-c3nc(-c4ccccc4)nc(-n4c5ccccc5c5c(-c6cccc7c6-c6ccccc6C76c7ccccc7-c7ccccc76)cccc54)n3)cc2)cc1 ; c1ccc(-c2cccc(-c3nc(-c4ccccc4)nc(-n4c5ccccc5c5c(-c6cccc7c6-c6ccccc6C76c7ccccc7-c7ccccc76)cccc54)n3)c2)cc1 ; c1ccc(-c2nc(-c3ccccc3-c3ccccc3)nc(-n3c4ccccc4c4c(-c5cccc6c5-c5ccccc5C65c6ccccc6-c6ccccc65)cccc43)n2)cc1] 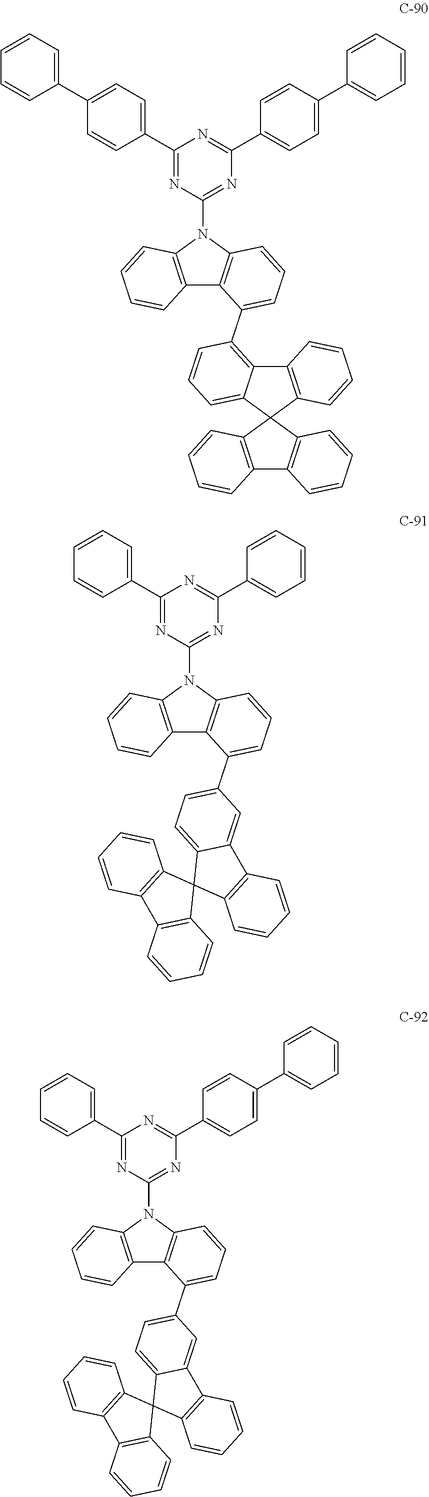 [SMILES: c1ccc(-c2ccc(-c3nc(-c4ccc(-c5ccccc5)cc4)nc(-n4c5ccccc5c5c(-c6cccc7c6-c6ccccc6C76c7ccccc7-c7ccccc76)cccc54)n3)cc2)cc1 ; c1ccc(-c2nc(-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccccc4)nc(-n4c5ccccc5c5c(-c6ccc7c(c6)-c6ccccc6C76c7ccccc7-c7ccccc76)cccc54)n3)cc2)cc1] 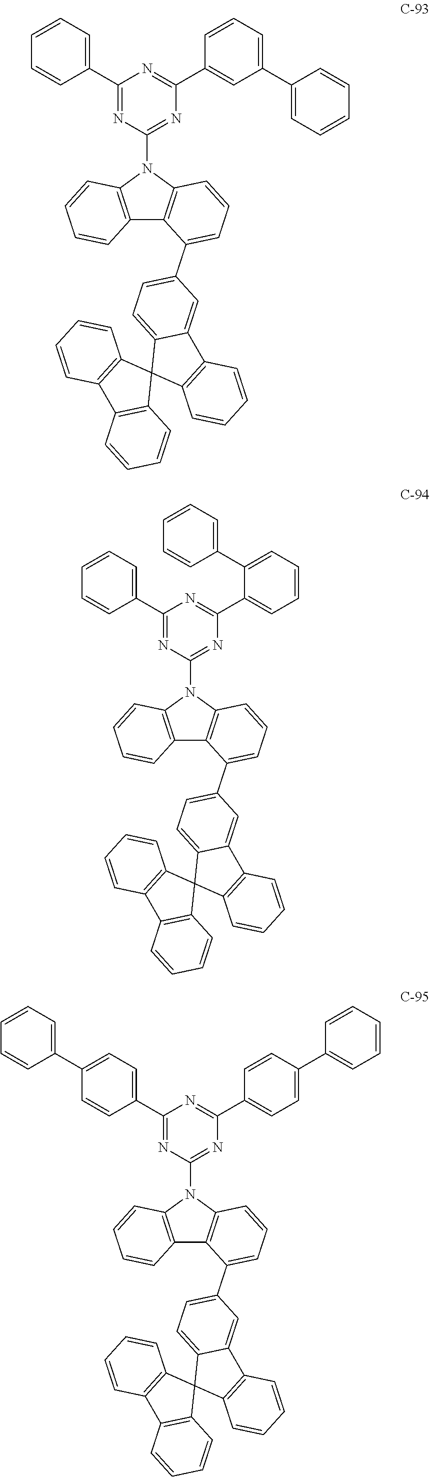 [SMILES: c1ccc(-c2cccc(-c3nc(-c4ccccc4)nc(-n4c5ccccc5c5c(-c6ccc7c(c6)-c6ccccc6C76c7ccccc7-c7ccccc76)cccc54)n3)c2)cc1 ; c1ccc(-c2nc(-c3ccccc3-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccc(-c5ccccc5)cc4)nc(-n4c5ccccc5c5c(-c6ccc7c(c6)-c6ccccc6C76c7ccccc7-c7ccccc76)cccc54)n3)cc2)cc1] 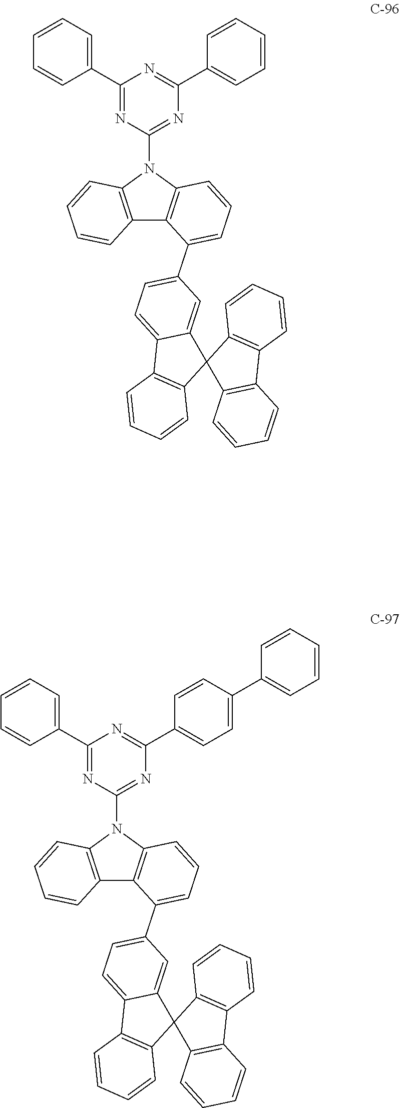 [SMILES: c1ccc(-c2nc(-c3ccccc3)nc(-n3c4ccccc4c4c(-c5ccc6c(c5)C5(c7ccccc7-c7ccccc75)c5ccccc5-6)cccc43)n2)cc1 ; c1ccc(-c2ccc(-c3nc(-c4ccccc4)nc(-n4c5ccccc5c5c(-c6ccc7c(c6)C6(c8ccccc8-c8ccccc86)c6ccccc6-7)cccc54)n3)cc2)cc1] 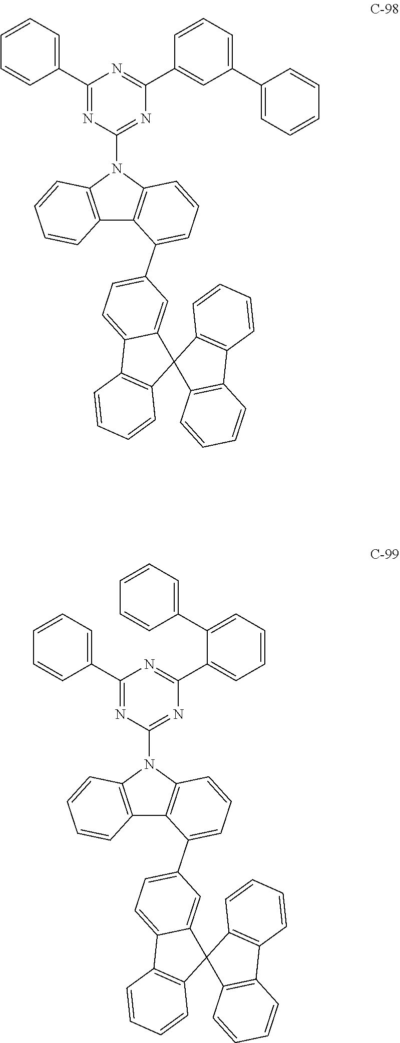 [SMILES: c1ccc(-c2cccc(-c3nc(-c4ccccc4)nc(-n4c5ccccc5c5c(-c6ccc7c(c6)C6(c8ccccc8-c8ccccc86)c6ccccc6-7)cccc54)n3)c2)cc1 ; c1ccc(-c2nc(-c3ccccc3-c3ccccc3)nc(-n3c4ccccc4c4c(-c5ccc6c(c5)C5(c7ccccc7-c7ccccc75)c5ccccc5-6)cccc43)n2)cc1] 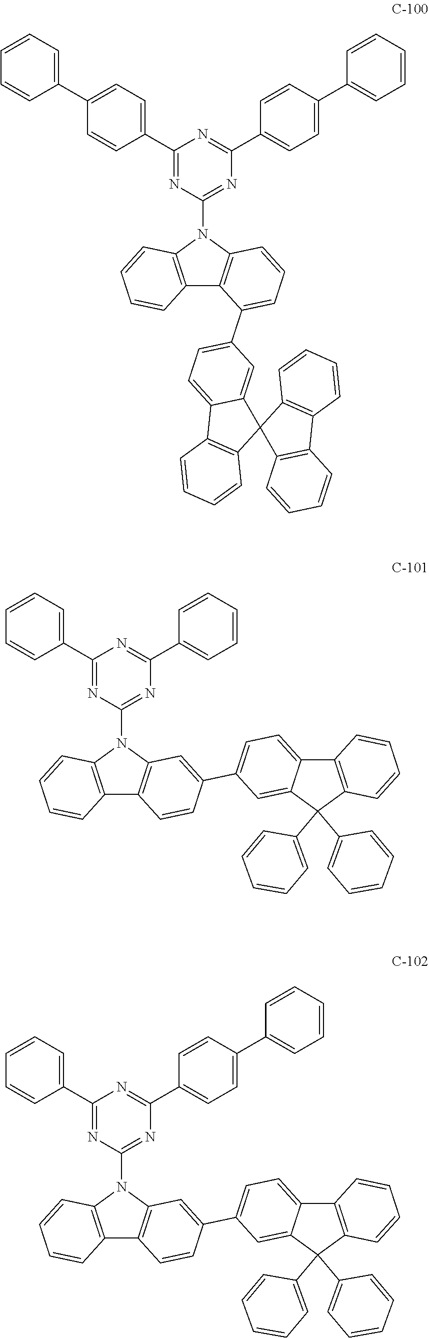 [SMILES: c1ccc(-c2ccc(-c3nc(-c4ccc(-c5ccccc5)cc4)nc(-n4c5ccccc5c5c(-c6ccc7c(c6)C6(c8ccccc8-c8ccccc86)c6ccccc6-7)cccc54)n3)cc2)cc1 ; c1ccc(-c2nc(-c3ccccc3)nc(-n3c4ccccc4c4ccc(-c5ccc6c(c5)C(c5ccccc5)(c5ccccc5)c5ccccc5-6)cc43)n2)cc1 ; c1ccc(-c2ccc(-c3nc(-c4ccccc4)nc(-n4c5ccccc5c5ccc(-c6ccc7c(c6)C(c6ccccc6)(c6ccccc6)c6ccccc6-7)cc54)n3)cc2)cc1] 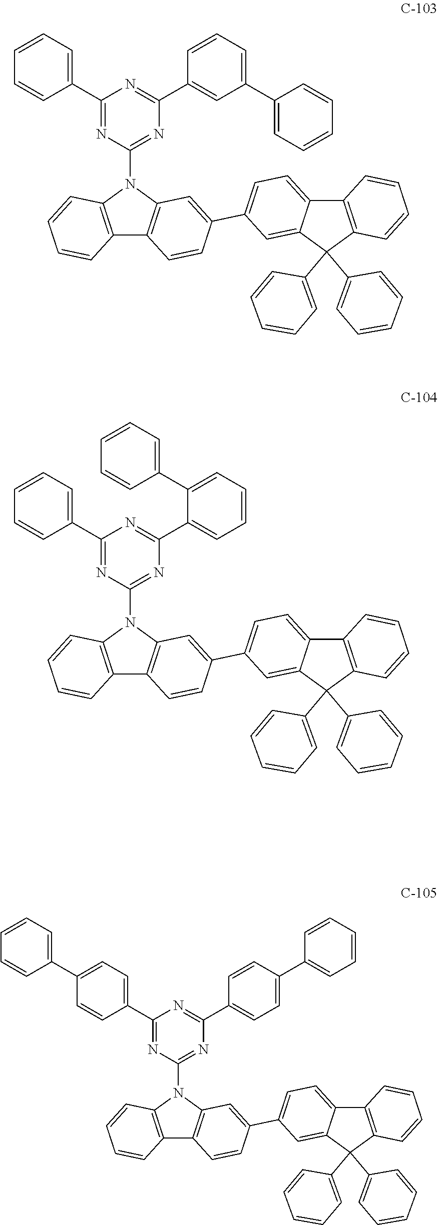 [SMILES: c1ccc(-c2cccc(-c3nc(-c4ccccc4)nc(-n4c5ccccc5c5ccc(-c6ccc7c(c6)C(c6ccccc6)(c6ccccc6)c6ccccc6-7)cc54)n3)c2)cc1 ; c1ccc(-c2nc(-c3ccccc3-c3ccccc3)nc(-n3c4ccccc4c4ccc(-c5ccc6c(c5)C(c5ccccc5)(c5ccccc5)c5ccccc5-6)cc43)n2)cc1 ; c1ccc(-c2ccc(-c3nc(-c4ccc(-c5ccccc5)cc4)nc(-n4c5ccccc5c5ccc(-c6ccc7c(c6)C(c6ccccc6)(c6ccccc6)c6ccccc6-7)cc54)n3)cc2)cc1] 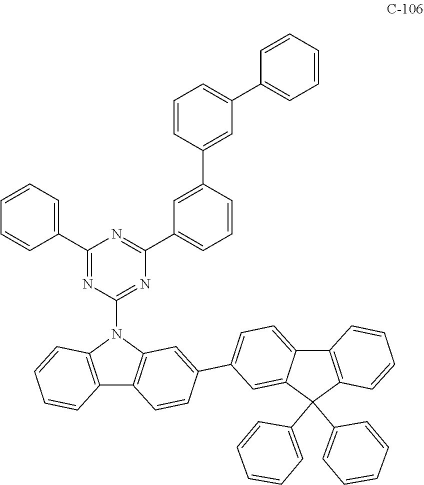 [SMILES: c1ccc(-c2cccc(-c3cccc(-c4nc(-c5ccccc5)nc(-n5c6ccccc6c6ccc(-c7ccc8c(c7)C(c7ccccc7)(c7ccccc7)c7ccccc7-8)cc65)n4)c3)c2)cc1] 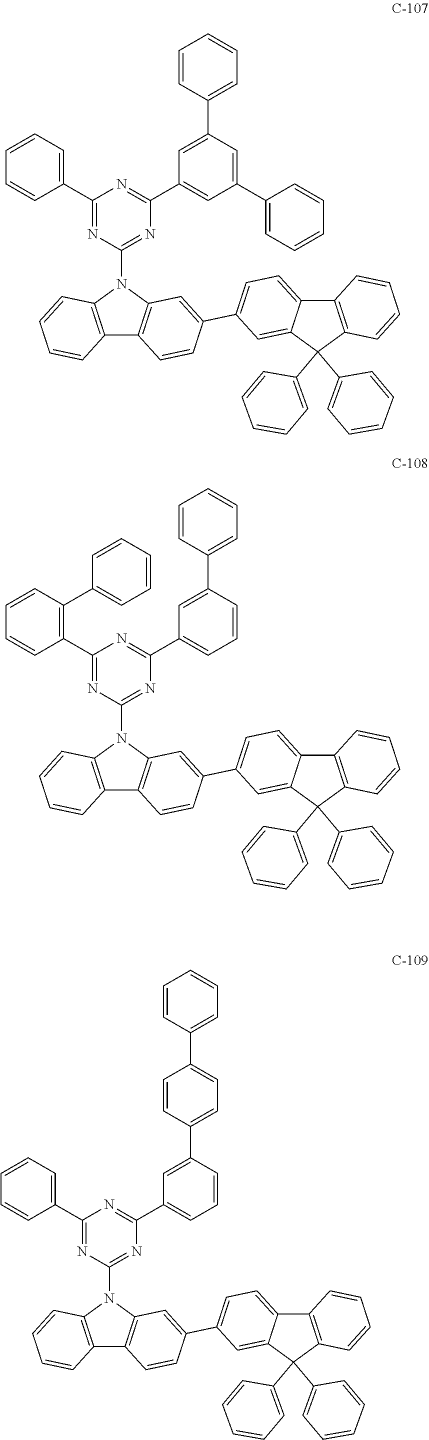 [SMILES: c1ccc(-c2cc(-c3ccccc3)cc(-c3nc(-c4ccccc4)nc(-n4c5ccccc5c5ccc(-c6ccc7c(c6)C(c6ccccc6)(c6ccccc6)c6ccccc6-7)cc54)n3)c2)cc1 ; c1ccc(-c2cccc(-c3nc(-c4ccccc4-c4ccccc4)nc(-n4c5ccccc5c5ccc(-c6ccc7c(c6)C(c6ccccc6)(c6ccccc6)c6ccccc6-7)cc54)n3)c2)cc1 ; c1ccc(-c2ccc(-c3cccc(-c4nc(-c5ccccc5)nc(-n5c6ccccc6c6ccc(-c7ccc8c(c7)C(c7ccccc7)(c7ccccc7)c7ccccc7-8)cc65)n4)c3)cc2)cc1] 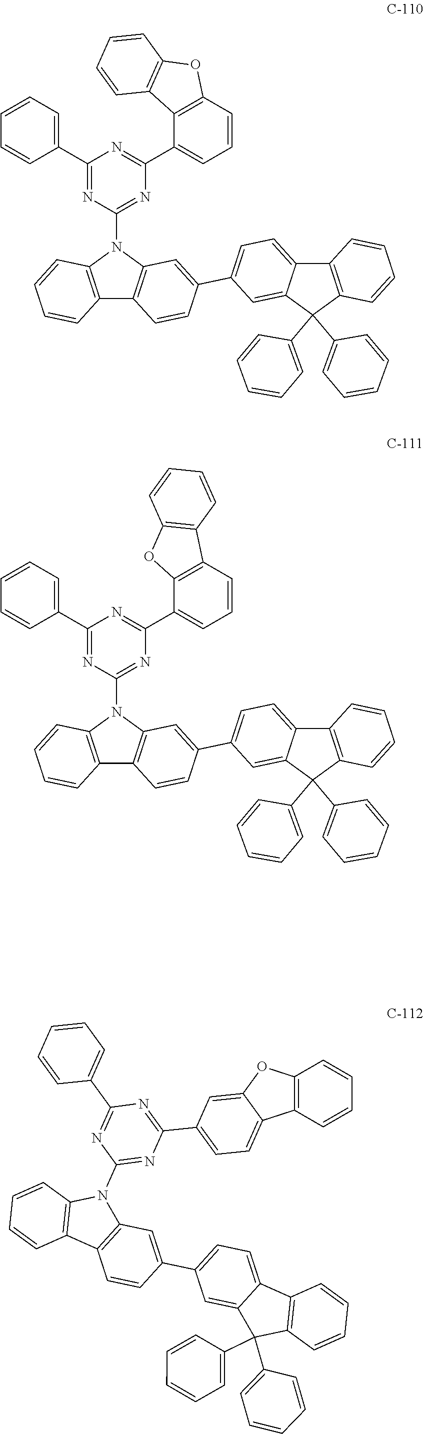 [SMILES: c1ccc(-c2nc(-c3cccc4oc5ccccc5c34)nc(-n3c4ccccc4c4ccc(-c5ccc6c(c5)C(c5ccccc5)(c5ccccc5)c5ccccc5-6)cc43)n2)cc1 ; c1ccc(-c2nc(-c3cccc4c3oc3ccccc34)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccc4c(c3)oc3ccccc34)nc(-n3c4ccccc4c4ccc(-c5ccc6c(c5)C(c5ccccc5)(c5ccccc5)c5ccccc5-6)cc43)n2)cc1] 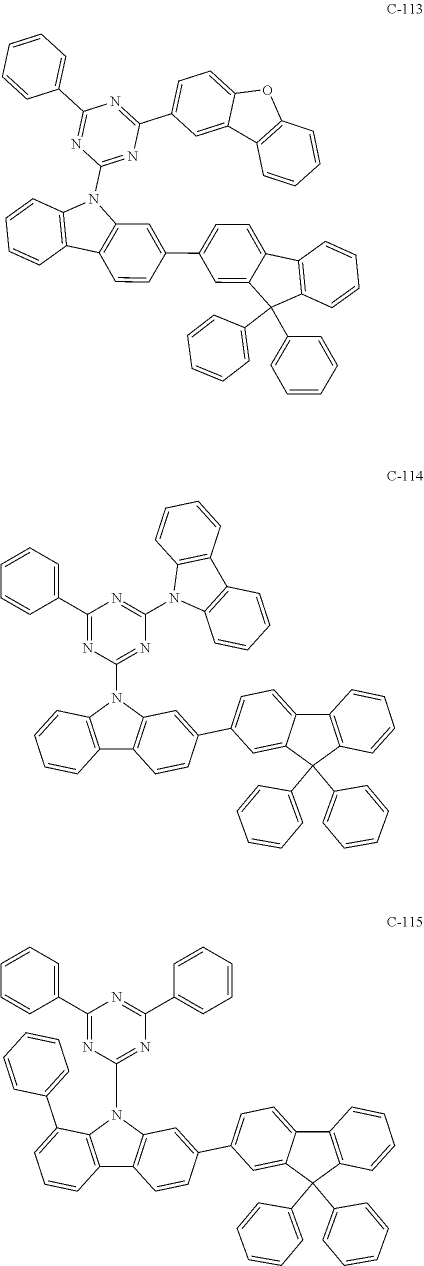 [SMILES: c1ccc(-c2nc(-c3ccc4oc5ccccc5c4c3)nc(-n3c4ccccc4c4ccc(-c5ccc6c(c5)C(c5ccccc5)(c5ccccc5)c5ccccc5-6)cc43)n2)cc1 ; c1ccc(-c2nc(-n3c4ccccc4c4ccccc43)nc(-n3c4ccccc4c4ccc(-c5ccc6c(c5)C(c5ccccc5)(c5ccccc5)c5ccccc5-6)cc43)n2)cc1 ; c1ccc(-c2nc(-c3ccccc3)nc(-n3c4cc(-c5ccc6c(c5)C(c5ccccc5)(c5ccccc5)c5ccccc5-6)ccc4c4cccc(-c5ccccc5)c43)n2)cc1] 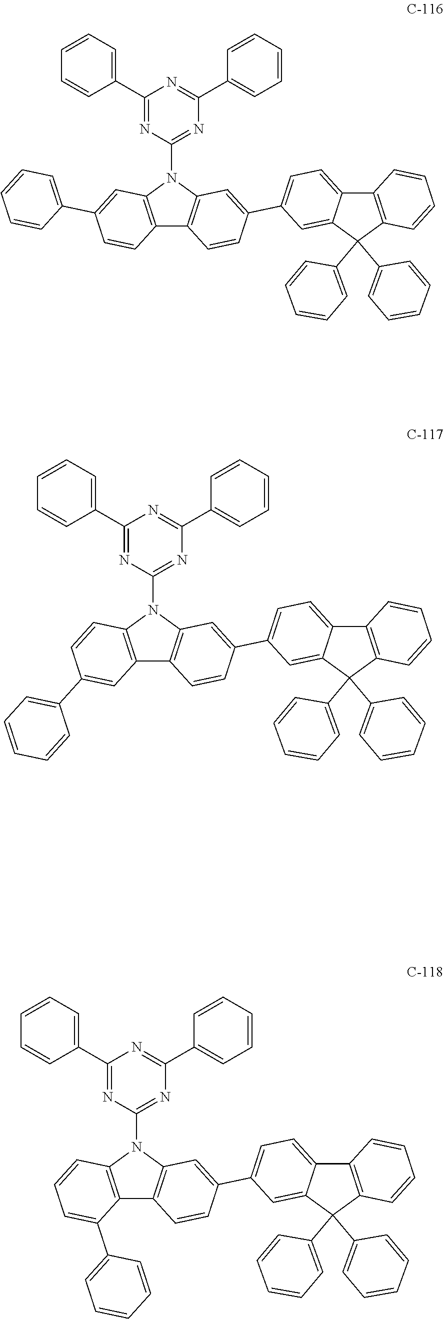 [SMILES: c1ccc(-c2ccc3c4ccc(-c5ccc6c(c5)C(c5ccccc5)(c5ccccc5)c5ccccc5-6)cc4n(-c4nc(-c5ccccc5)nc(-c5ccccc5)n4)c3c2)cc1 ; c1ccc(-c2ccc3c(c2)c2ccc(-c4ccc5c(c4)C(c4ccccc4)(c4ccccc4)c4ccccc4-5)cc2n3-c2nc(-c3ccccc3)nc(-c3ccccc3)n2)cc1 ; c1ccc(-c2nc(-c3ccccc3)nc(-n3c4cc(-c5ccc6c(c5)C(c5ccccc5)(c5ccccc5)c5ccccc5-6)ccc4c4c(-c5ccccc5)cccc43)n2)cc1] 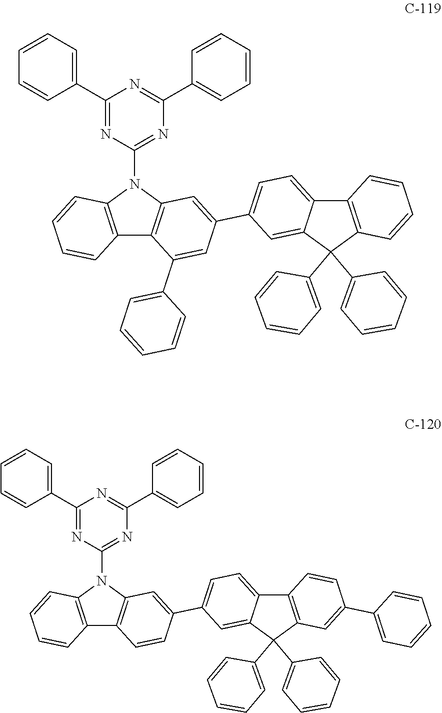 [SMILES: c1ccc(-c2nc(-c3ccccc3)nc(-n3c4ccccc4c4c(-c5ccccc5)cc(-c5ccc6c(c5)C(c5ccccc5)(c5ccccc5)c5ccccc5-6)cc43)n2)cc1 ; c1ccc(-c2ccc3c(c2)C(c2ccccc2)(c2ccccc2)c2cc(-c4ccc5c6ccccc6n(-c6nc(-c7ccccc7)nc(-c7ccccc7)n6)c5c4)ccc2-3)cc1] 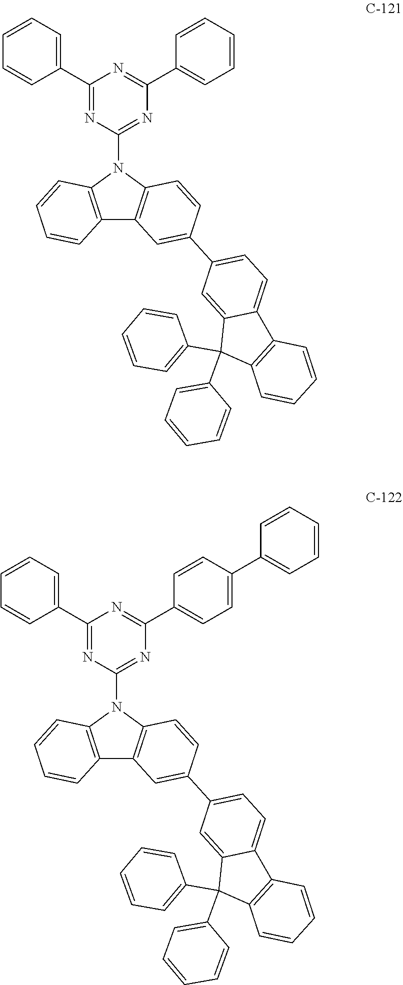 [SMILES: c1ccc(-c2nc(-c3ccccc3)nc(-n3c4ccccc4c4cc(-c5ccc6c(c5)C(c5ccccc5)(c5ccccc5)c5ccccc5-6)ccc43)n2)cc1 ; c1ccc(-c2ccc(-c3nc(-c4ccccc4)nc(-n4c5ccccc5c5cc(-c6ccc7c(c6)C(c6ccccc6)(c6ccccc6)c6ccccc6-7)ccc54)n3)cc2)cc1] 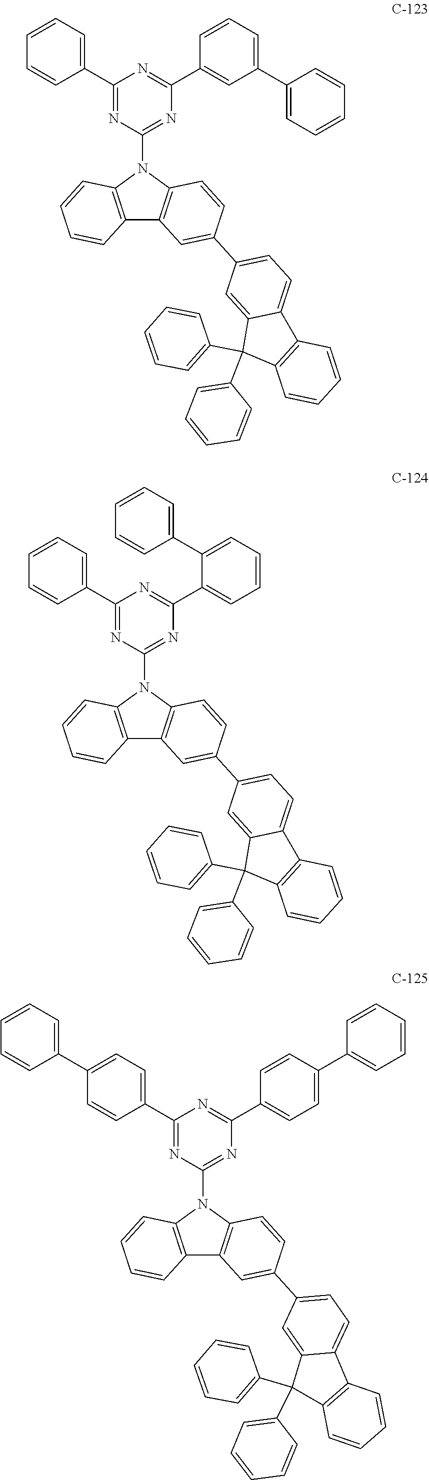 [SMILES: c1ccc(-c2cccc(-c3nc(-c4ccccc4)nc(-n4c5ccccc5c5cc(-c6ccc7c(c6)C(c6ccccc6)(c6ccccc6)c6ccccc6-7)ccc54)n3)c2)cc1 ; c1ccc(-c2nc(-c3ccccc3-c3ccccc3)nc(-n3c4ccccc4c4cc(-c5ccc6c(c5)C(c5ccccc5)(c5ccccc5)c5ccccc5-6)ccc43)n2)cc1 ; c1ccc(-c2ccc(-c3nc(-c4ccc(-c5ccccc5)cc4)nc(-n4c5ccccc5c5cc(-c6ccc7c(c6)C(c6ccccc6)(c6ccccc6)c6ccccc6-7)ccc54)n3)cc2)cc1] 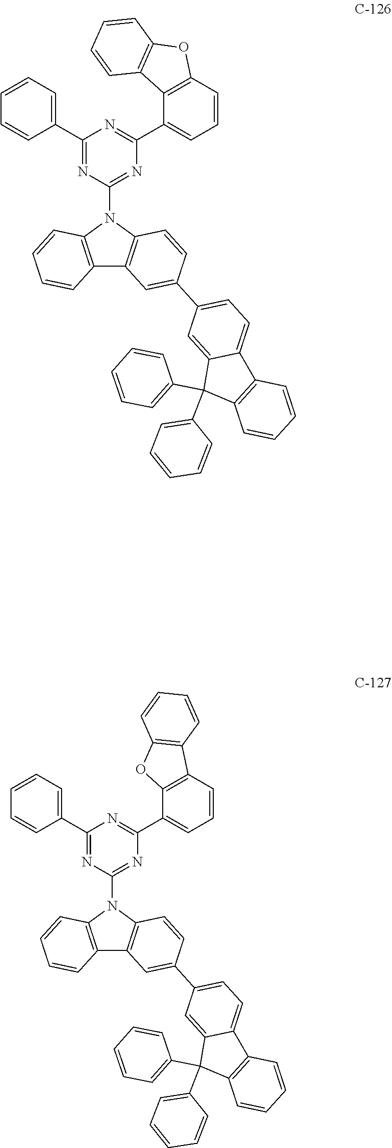 [SMILES: c1ccc(-c2nc(-c3cccc4oc5ccccc5c34)nc(-n3c4ccccc4c4cc(-c5ccc6c(c5)C(c5ccccc5)(c5ccccc5)c5ccccc5-6)ccc43)n2)cc1 ; c1ccc(-c2nc(-c3cccc4c3oc3ccccc34)nc(-n3c4ccccc4c4cc(-c5ccc6c(c5)C(c5ccccc5)(c5ccccc5)c5ccccc5-6)ccc43)n2)cc1] 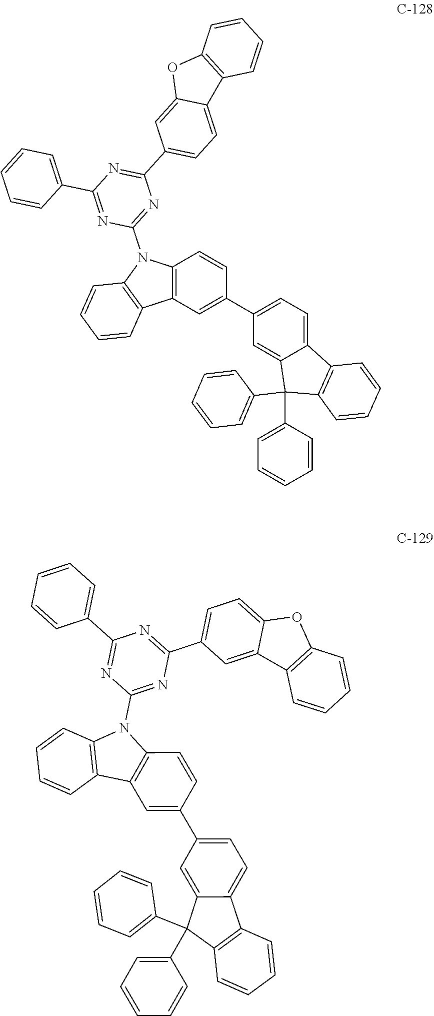 [SMILES: c1ccc(-c2nc(-c3ccc4c(c3)oc3ccccc34)nc(-n3c4ccccc4c4cc(-c5ccc6c(c5)C(c5ccccc5)(c5ccccc5)c5ccccc5-6)ccc43)n2)cc1 ; c1ccc(-c2nc(-c3ccc4oc5ccccc5c4c3)nc(-n3c4ccccc4c4cc(-c5ccc6c(c5)C(c5ccccc5)(c5ccccc5)c5ccccc5-6)ccc43)n2)cc1] 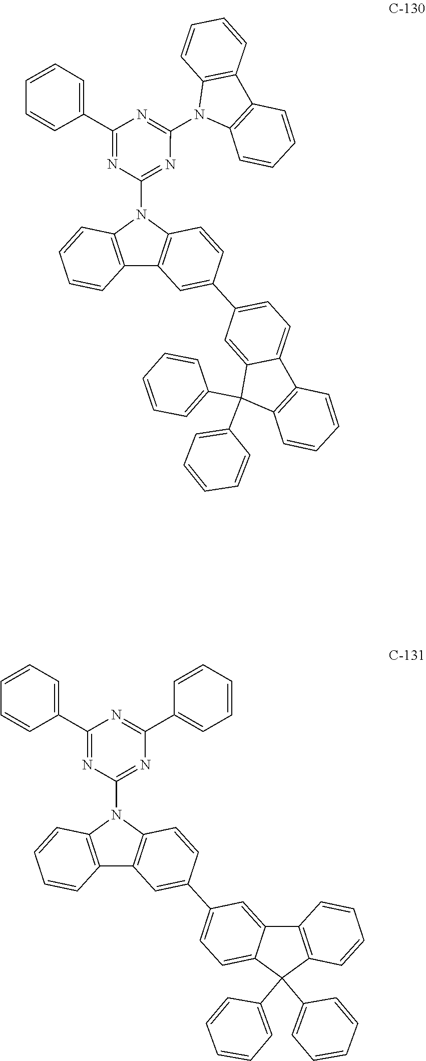 [SMILES: c1ccc(-c2nc(-n3c4ccccc4c4ccccc43)nc(-n3c4ccccc4c4cc(-c5ccc6c(c5)C(c5ccccc5)(c5ccccc5)c5ccccc5-6)ccc43)n2)cc1 ; c1ccc(-c2nc(-c3ccccc3)nc(-n3c4ccccc4c4cc(-c5ccc6c(c5)-c5ccccc5C6(c5ccccc5)c5ccccc5)ccc43)n2)cc1] 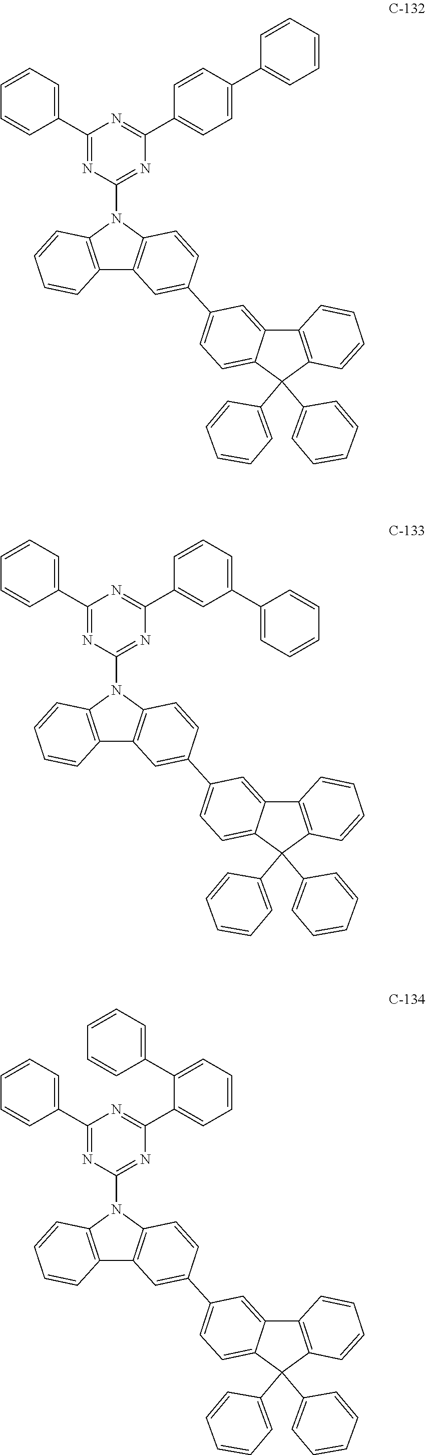 [SMILES: c1ccc(-c2ccc(-c3nc(-c4ccccc4)nc(-n4c5ccccc5c5cc(-c6ccc7c(c6)-c6ccccc6C7(c6ccccc6)c6ccccc6)ccc54)n3)cc2)cc1 ; c1ccc(-c2cccc(-c3nc(-c4ccccc4)nc(-n4c5ccccc5c5cc(-c6ccc7c(c6)-c6ccccc6C7(c6ccccc6)c6ccccc6)ccc54)n3)c2)cc1 ; c1ccc(-c2nc(-c3ccccc3-c3ccccc3)nc(-n3c4ccccc4c4cc(-c5ccc6c(c5)-c5ccccc5C6(c5ccccc5)c5ccccc5)ccc43)n2)cc1] 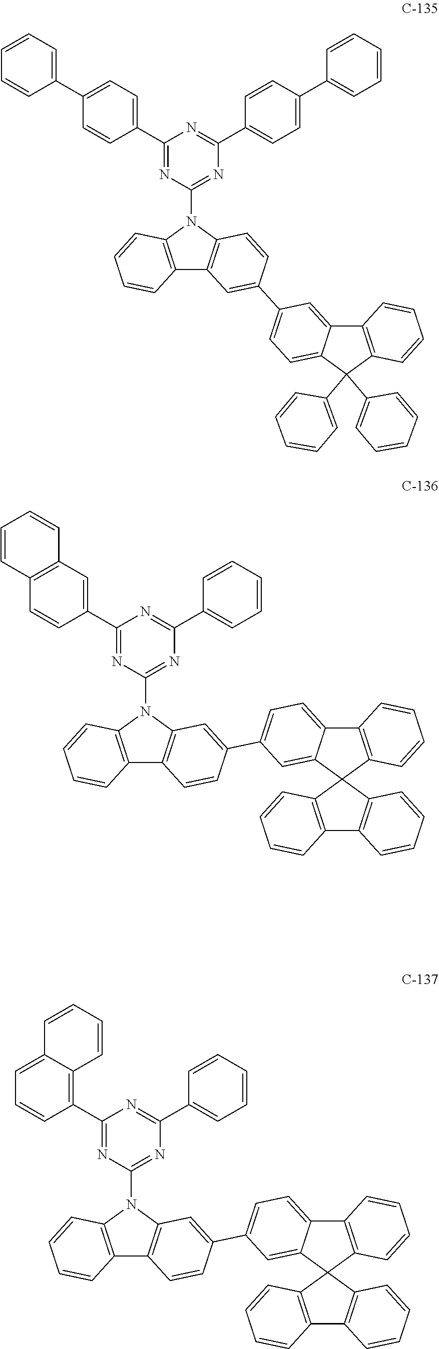 [SMILES: c1ccc(-c2ccc(-c3nc(-c4ccc(-c5ccccc5)cc4)nc(-n4c5ccccc5c5cc(-c6ccc7c(c6)-c6ccccc6C7(c6ccccc6)c6ccccc6)ccc54)n3)cc2)cc1 ; c1ccc(-c2nc(-c3ccc4ccccc4c3)nc(-n3c4ccccc4c4ccc(-c5ccc6c(c5)C5(c7ccccc7-c7ccccc75)c5ccccc5-6)cc43)n2)cc1 ; c1ccc(-c2nc(-c3cccc4ccccc34)nc(-n3c4ccccc4c4ccc(-c5ccc6c(c5)C5(c7ccccc7-c7ccccc75)c5ccccc5-6)cc43)n2)cc1] 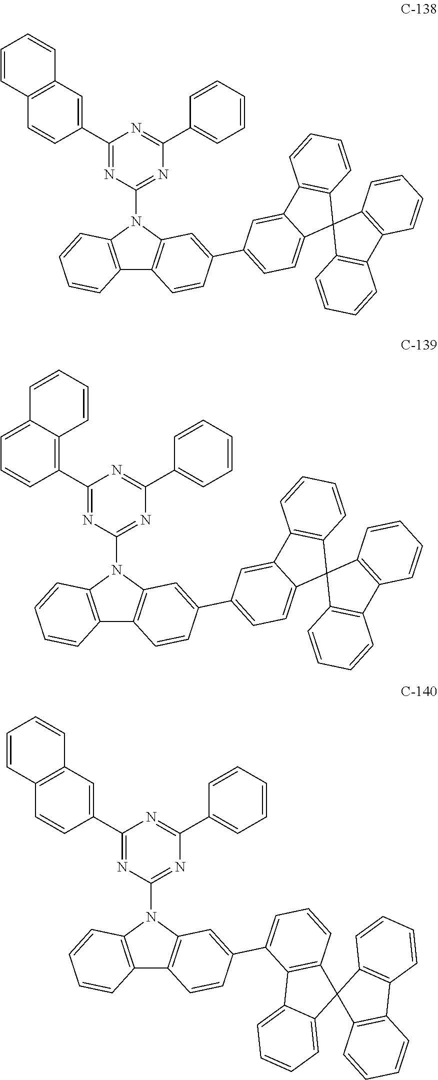 [SMILES: c1ccc(-c2nc(-c3ccc4ccccc4c3)nc(-n3c4ccccc4c4ccc(-c5ccc6c(c5)-c5ccccc5C65c6ccccc6-c6ccccc65)cc43)n2)cc1 ; c1ccc(-c2nc(-c3cccc4ccccc34)nc(-n3c4ccccc4c4ccc(-c5ccc6c(c5)-c5ccccc5C65c6ccccc6-c6ccccc65)cc43)n2)cc1 ; c1ccc(-c2nc(-c3ccc4ccccc4c3)nc(-n3c4ccccc4c4ccc(-c5cccc6c5-c5ccccc5C65c6ccccc6-c6ccccc65)cc43)n2)cc1] 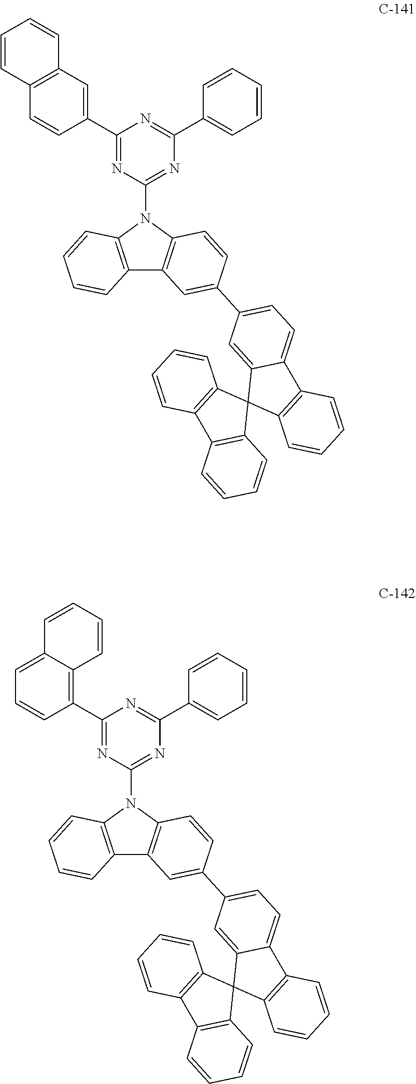 [SMILES: c1ccc(-c2nc(-c3ccc4ccccc4c3)nc(-n3c4ccccc4c4cc(-c5ccc6c(c5)C5(c7ccccc7-c7ccccc75)c5ccccc5-6)ccc43)n2)cc1 ; c1ccc(-c2nc(-c3cccc4ccccc34)nc(-n3c4ccccc4c4cc(-c5ccc6c(c5)C5(c7ccccc7-c7ccccc75)c5ccccc5-6)ccc43)n2)cc1] 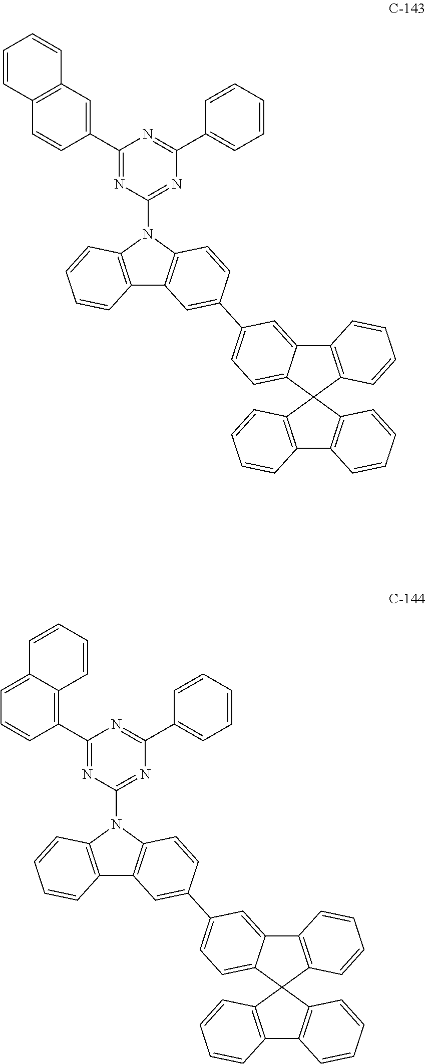 [SMILES: c1ccc(-c2nc(-c3ccc4ccccc4c3)nc(-n3c4ccccc4c4cc(-c5ccc6c(c5)-c5ccccc5C65c6ccccc6-c6ccccc65)ccc43)n2)cc1 ; c1ccc(-c2nc(-c3cccc4ccccc34)nc(-n3c4ccccc4c4cc(-c5ccc6c(c5)-c5ccccc5C65c6ccccc6-c6ccccc65)ccc43)n2)cc1] 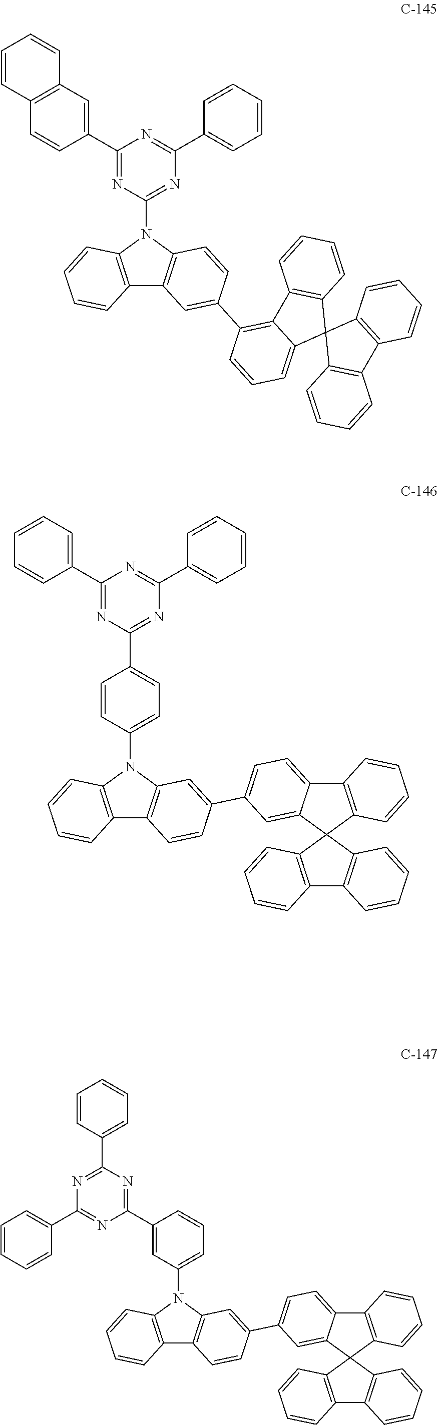 [SMILES: c1ccc(-c2nc(-c3ccc4ccccc4c3)nc(-n3c4ccccc4c4cc(-c5cccc6c5-c5ccccc5C65c6ccccc6-c6ccccc65)ccc43)n2)cc1 ; c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)cc3)n2)cc1 ; c1ccc(-c2nc(-c3ccccc3)nc(-c3cccc(-n4c5ccccc5c5ccc(-c6ccc7c(c6)C6(c8ccccc8-c8ccccc86)c6ccccc6-7)cc54)c3)n2)cc1] 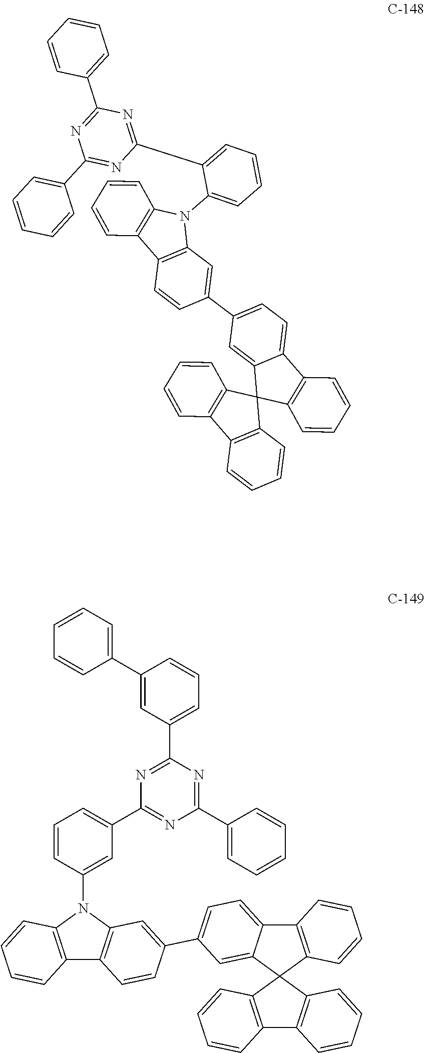 [SMILES: c1ccc(-c2nc(-c3ccccc3)nc(-c3ccccc3-n3c4ccccc4c4ccc(-c5ccc6c(c5)C5(c7ccccc7-c7ccccc75)c5ccccc5-6)cc43)n2)cc1 ; c1ccc(-c2cccc(-c3nc(-c4ccccc4)nc(-c4cccc(-n5c6ccccc6c6ccc(-c7ccc8c(c7)C7(c9ccccc9-c9ccccc97)c7ccccc7-8)cc65)c4)n3)c2)cc1] 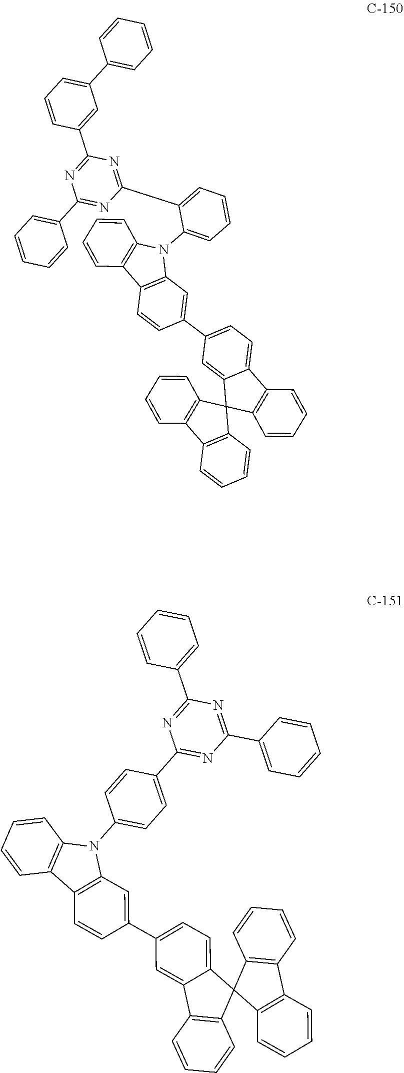 [SMILES: c1ccc(-c2cccc(-c3nc(-c4ccccc4)nc(-c4ccccc4-n4c5ccccc5c5ccc(-c6ccc7c(c6)C6(c8ccccc8-c8ccccc86)c6ccccc6-7)cc54)n3)c2)cc1 ; c1ccc(-c2nc(-c3ccccc3)nc(-c3ccc(-n4c5ccccc5c5ccc(-c6ccc7c(c6)-c6ccccc6C76c7ccccc7-c7ccccc76)cc54)cc3)n2)cc1] 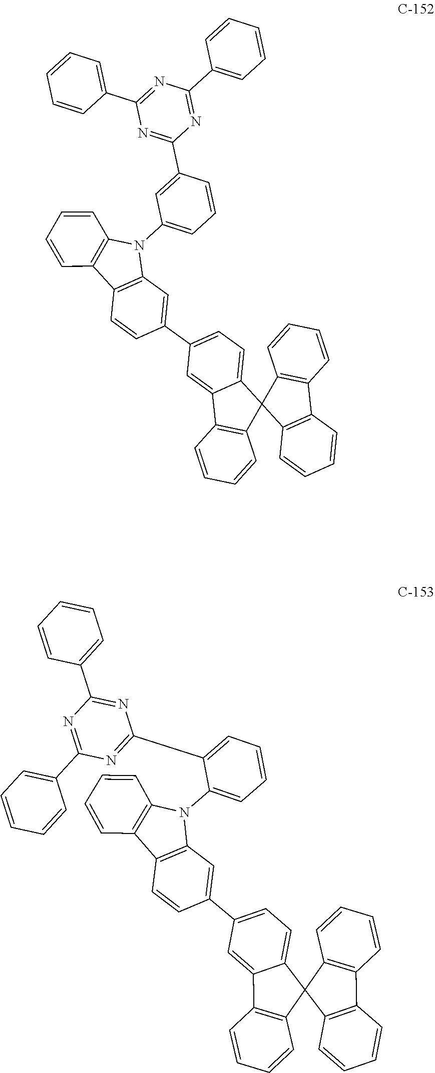 [SMILES: c1ccc(-c2nc(-c3ccccc3)nc(-c3cccc(-n4c5ccccc5c5ccc(-c6ccc7c(c6)-c6ccccc6C76c7ccccc7-c7ccccc76)cc54)c3)n2)cc1 ; c1ccc(-c2nc(-c3ccccc3)nc(-c3ccccc3-n3c4ccccc4c4ccc(-c5ccc6c(c5)-c5ccccc5C65c6ccccc6-c6ccccc65)cc43)n2)cc1] 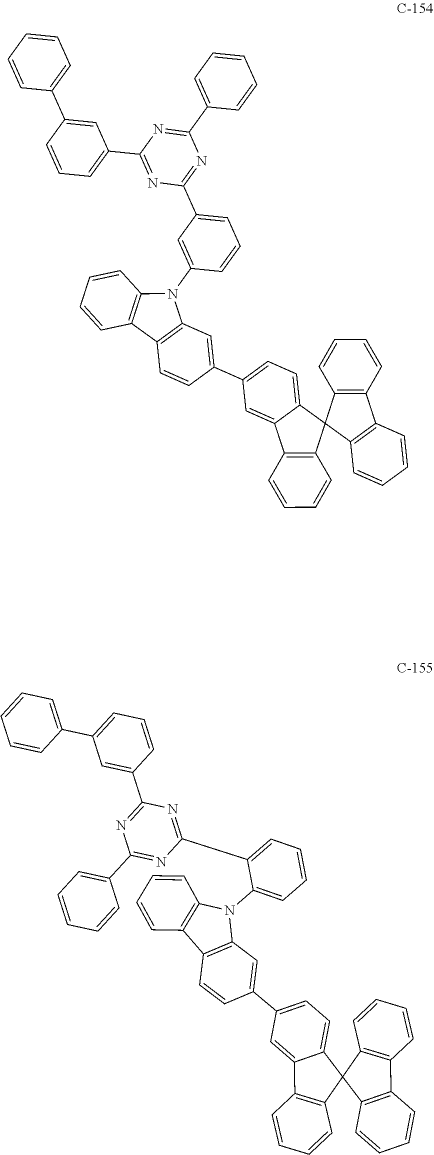 [SMILES: c1ccc(-c2cccc(-c3nc(-c4ccccc4)nc(-c4cccc(-n5c6ccccc6c6ccc(-c7ccc8c(c7)-c7ccccc7C87c8ccccc8-c8ccccc87)cc65)c4)n3)c2)cc1 ; c1ccc(-c2cccc(-c3nc(-c4ccccc4)nc(-c4ccccc4-n4c5ccccc5c5ccc(-c6ccc7c(c6)-c6ccccc6C76c7ccccc7-c7ccccc76)cc54)n3)c2)cc1] 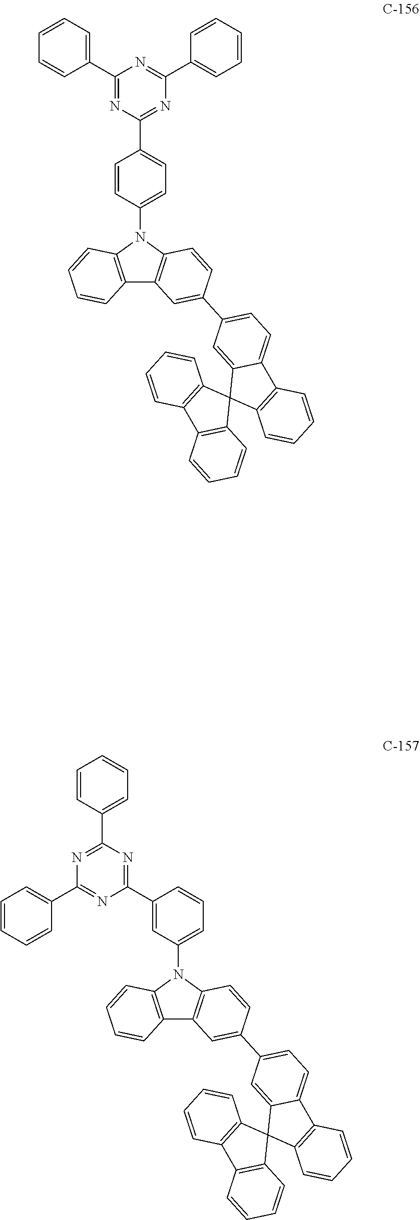 [SMILES: c1ccc(-c2nc(-c3ccccc3)nc(-c3ccc(-n4c5ccccc5c5cc(-c6ccc7c(c6)C6(c8ccccc8-c8ccccc86)c6ccccc6-7)ccc54)cc3)n2)cc1 ; c1ccc(-c2nc(-c3ccccc3)nc(-c3cccc(-n4c5ccccc5c5cc(-c6ccc7c(c6)C6(c8ccccc8-c8ccccc86)c6ccccc6-7)ccc54)c3)n2)cc1] 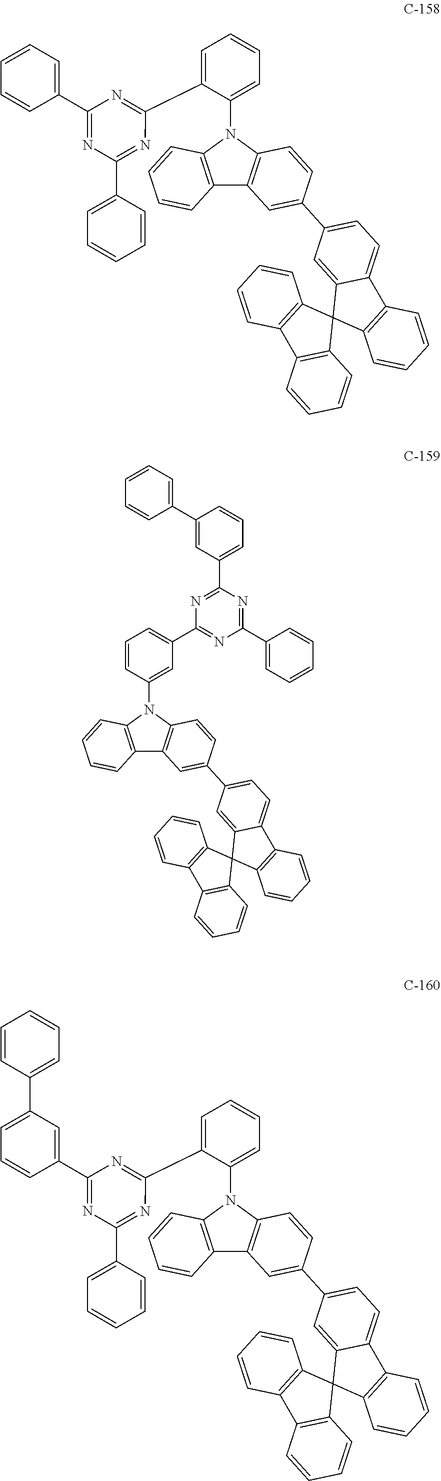 [SMILES: c1ccc(-c2nc(-c3ccccc3)nc(-c3ccccc3-n3c4ccccc4c4cc(-c5ccc6c(c5)C5(c7ccccc7-c7ccccc75)c5ccccc5-6)ccc43)n2)cc1 ; c1ccc(-c2cccc(-c3nc(-c4ccccc4)nc(-c4cccc(-n5c6ccccc6c6cc(-c7ccc8c(c7)C7(c9ccccc9-c9ccccc97)c7ccccc7-8)ccc65)c4)n3)c2)cc1 ; c1ccc(-c2cccc(-c3nc(-c4ccccc4)nc(-c4ccccc4-n4c5ccccc5c5cc(-c6ccc7c(c6)C6(c8ccccc8-c8ccccc86)c6ccccc6-7)ccc54)n3)c2)cc1] 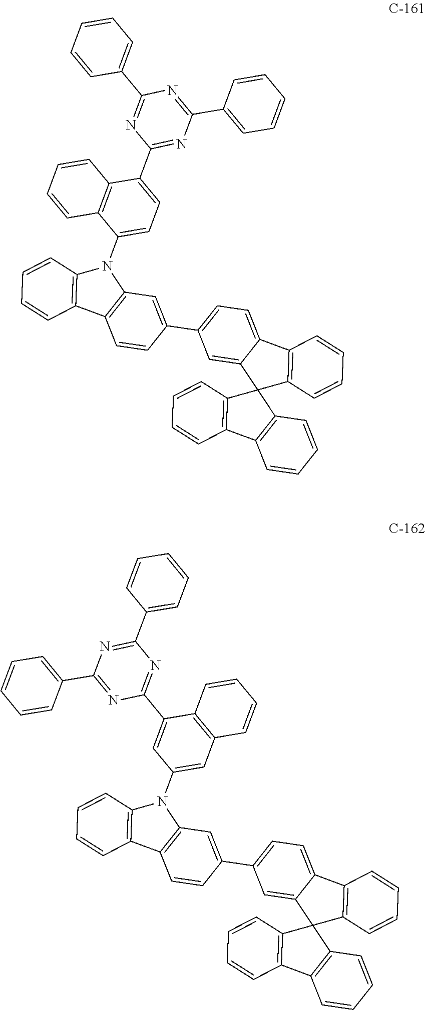 [SMILES: c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)c4ccccc34)n2)cc1 ; c1ccc(-c2nc(-c3ccccc3)nc(-c3cc(-n4c5ccccc5c5ccc(-c6ccc7c(c6)C6(c8ccccc8-c8ccccc86)c6ccccc6-7)cc54)cc4ccccc34)n2)cc1] 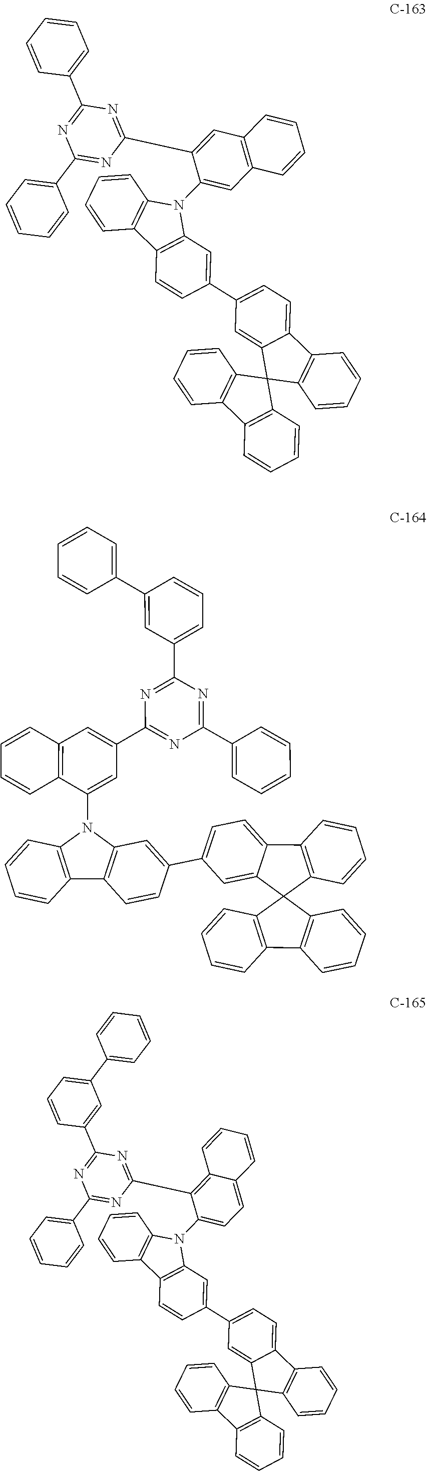 [SMILES: c1ccc(-c2nc(-c3ccccc3)nc(-c3cc4ccccc4cc3-n3c4ccccc4c4ccc(-c5ccc6c(c5)C5(c7ccccc7-c7ccccc75)c5ccccc5-6)cc43)n2)cc1 ; c1ccc(-c2cccc(-c3nc(-c4ccccc4)nc(-c4cc(-n5c6ccccc6c6ccc(-c7ccc8c(c7)C7(c9ccccc9-c9ccccc97)c7ccccc7-8)cc65)c5ccccc5c4)n3)c2)cc1 ; c1ccc(-c2cccc(-c3nc(-c4ccccc4)nc(-c4c(-n5c6ccccc6c6ccc(-c7ccc8c(c7)C7(c9ccccc9-c9ccccc97)c7ccccc7-8)cc65)ccc5ccccc45)n3)c2)cc1] 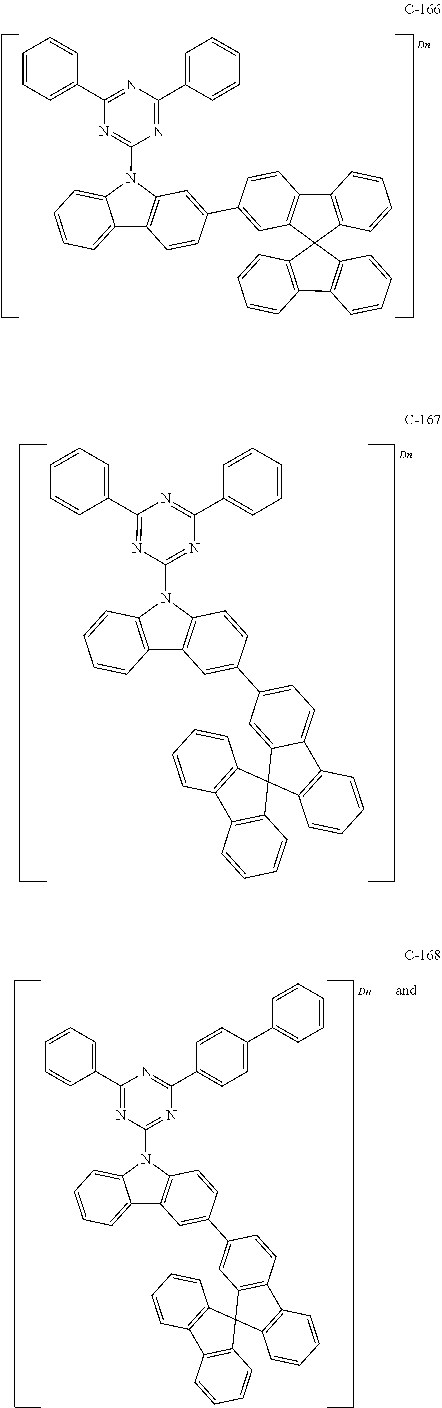 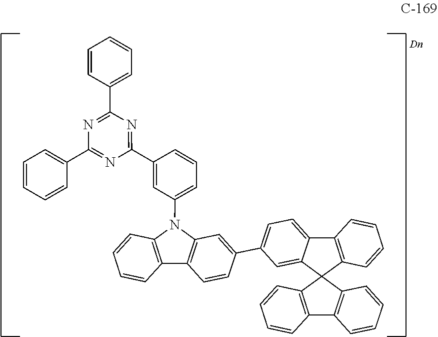
In the compounds above, Dn means that n number of hydrogens is replaced with deuterium, wherein n represents an integer of 1 or more and the upper limit of n is determined by the number of hydrogens that can be substituted in each compound.

13. The plurality of host materials according to claim 1, wherein the compound represented by the Formula 2 is selected from the following compounds: 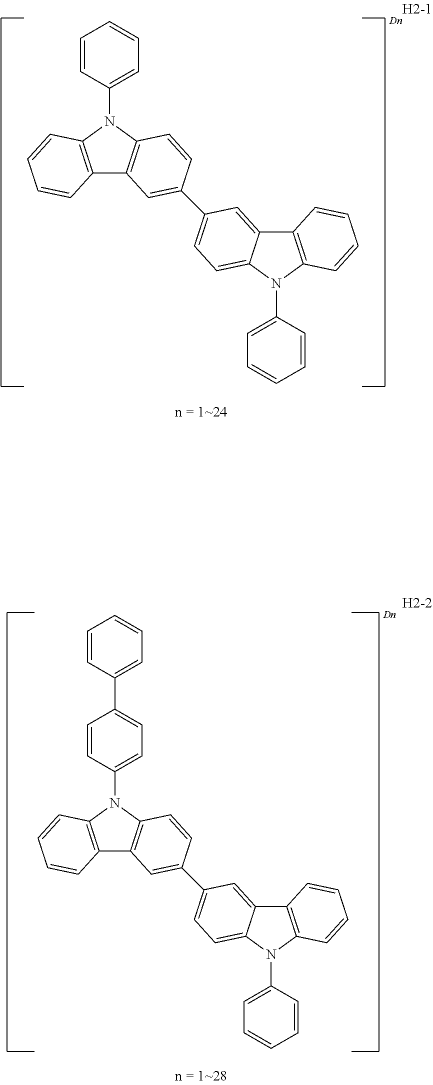 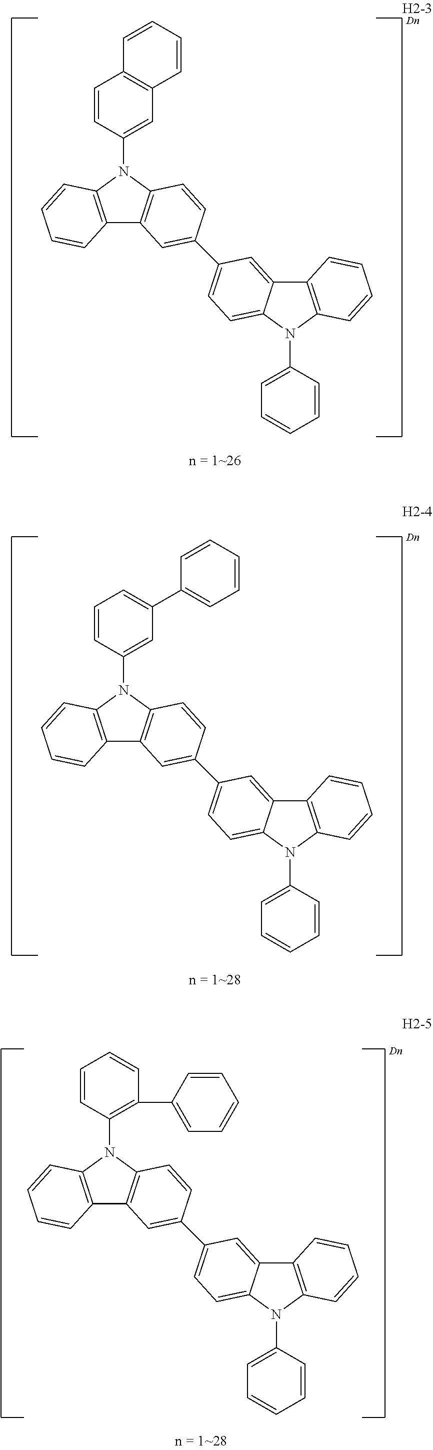 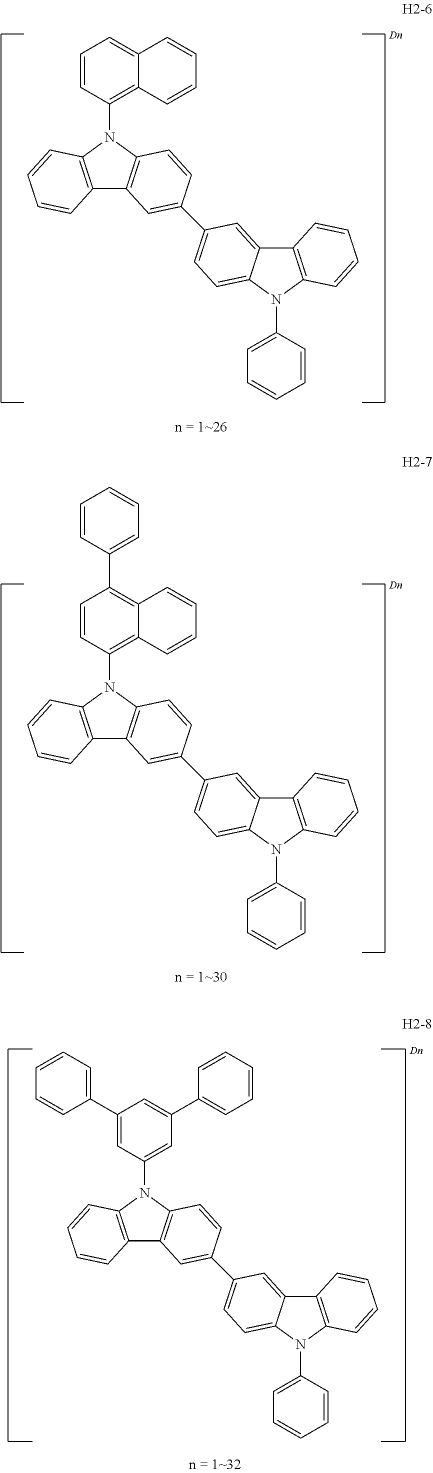 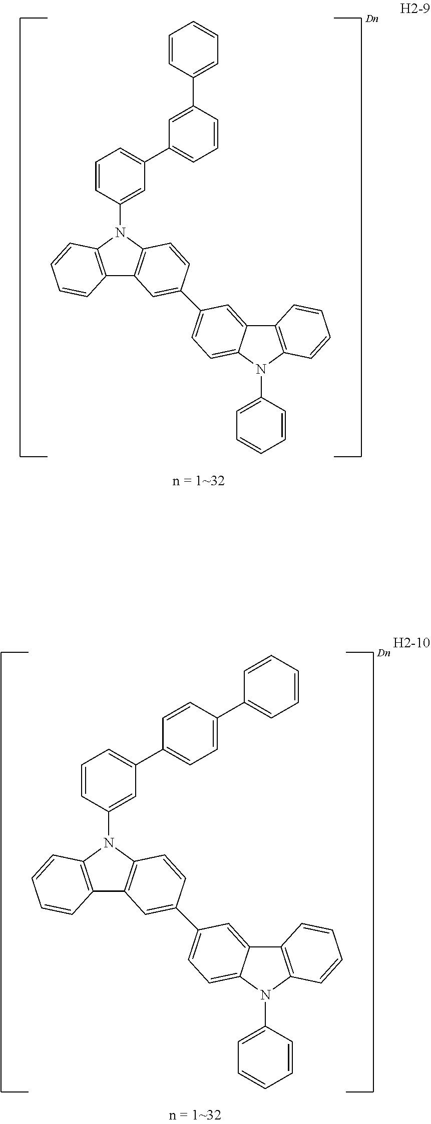 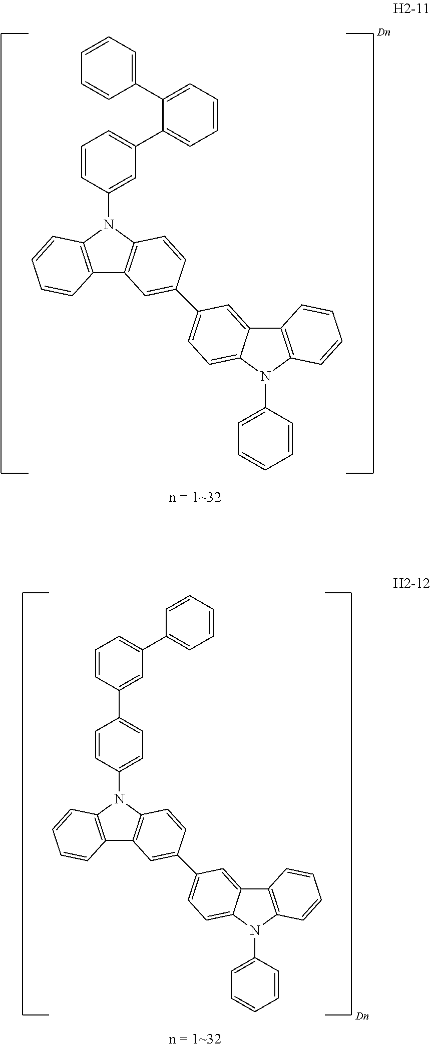 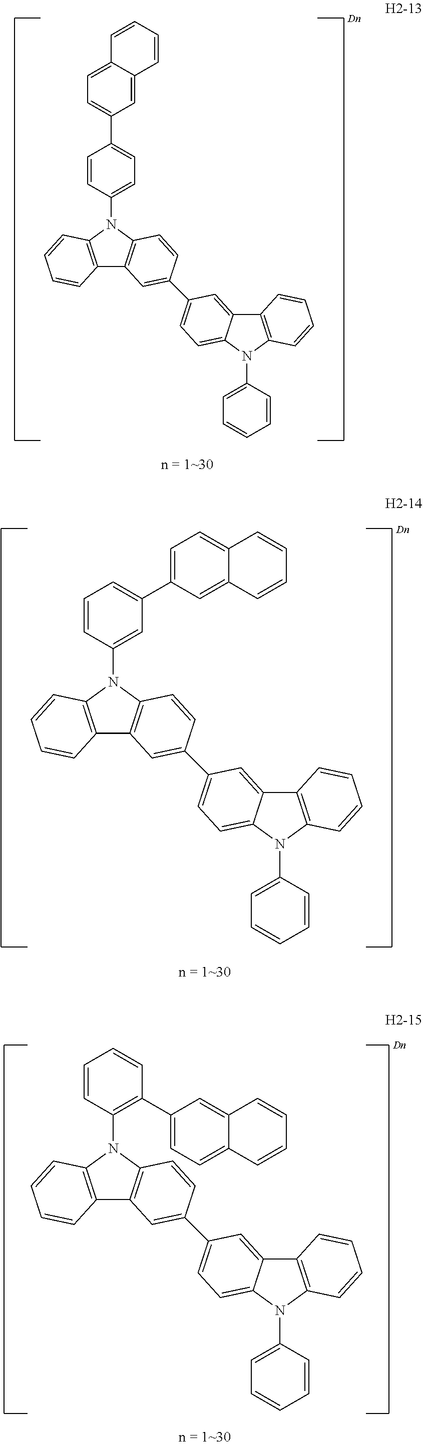 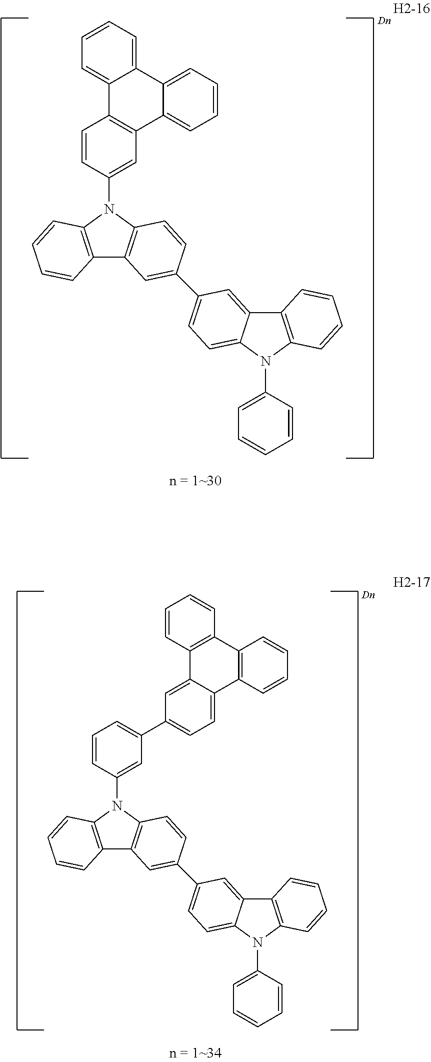 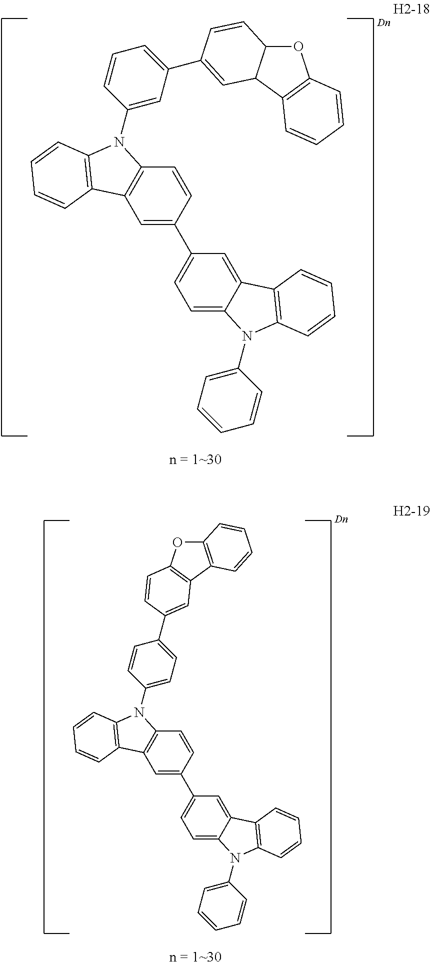 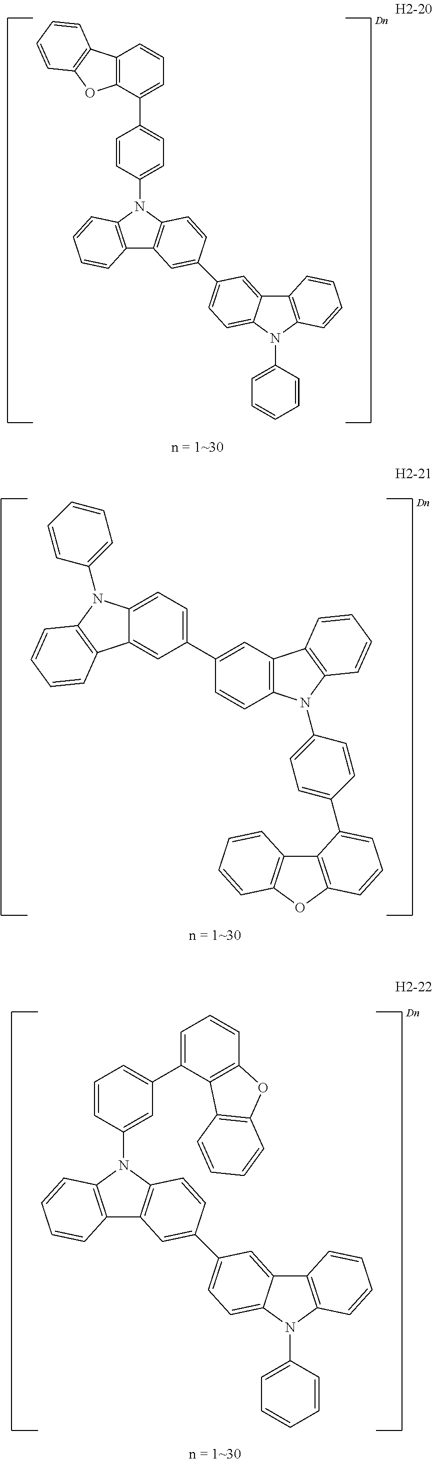 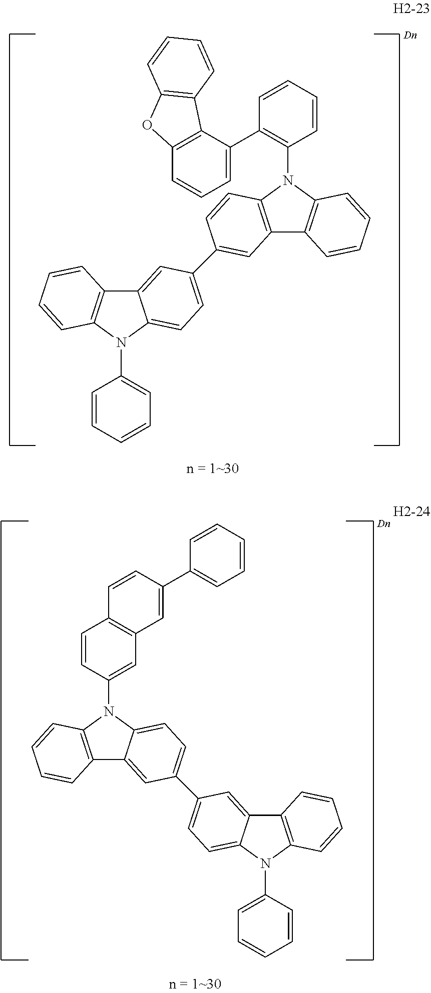 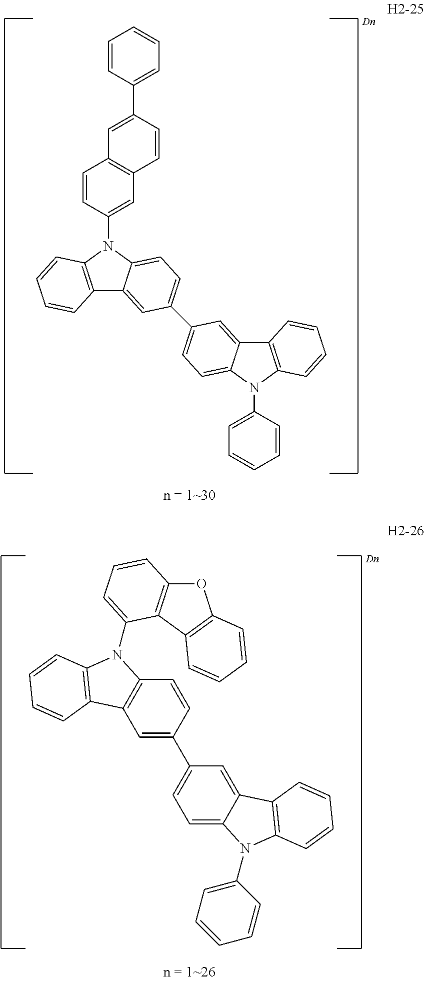 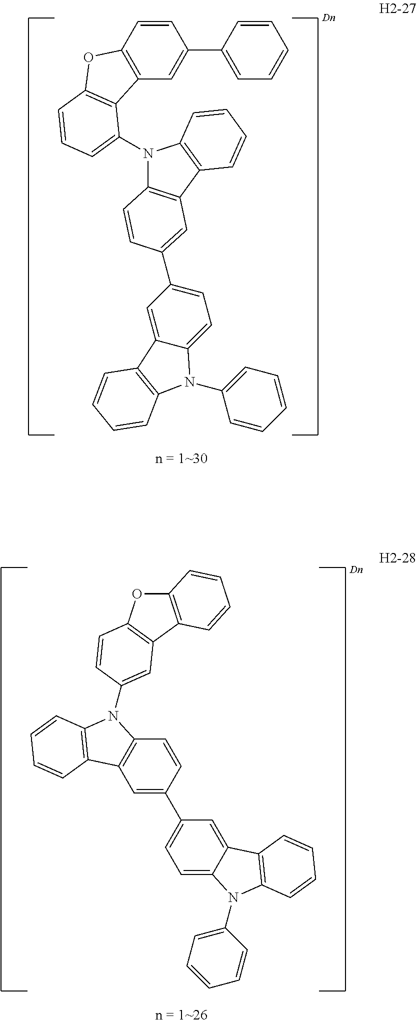 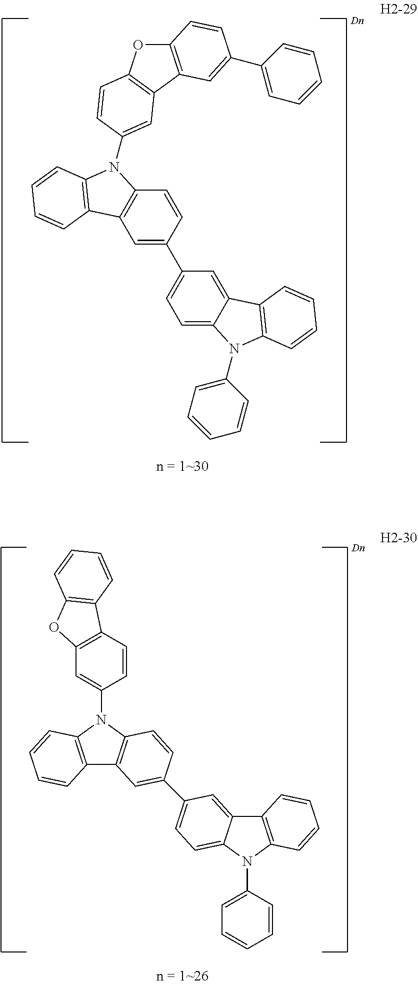 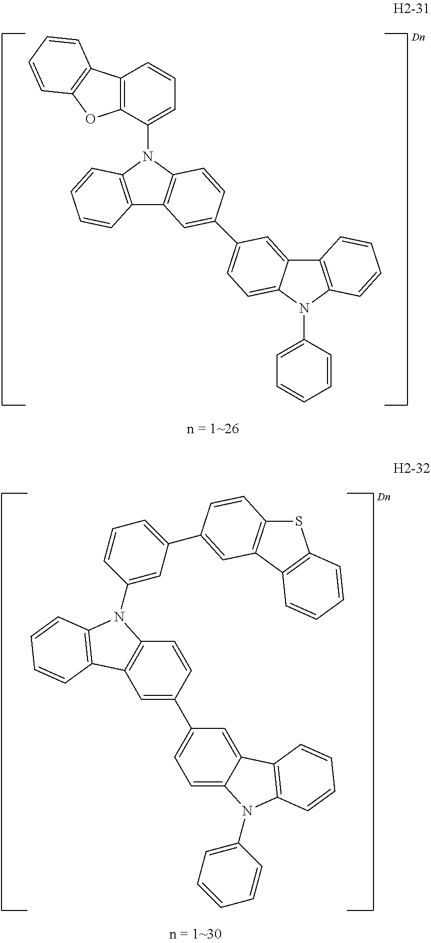 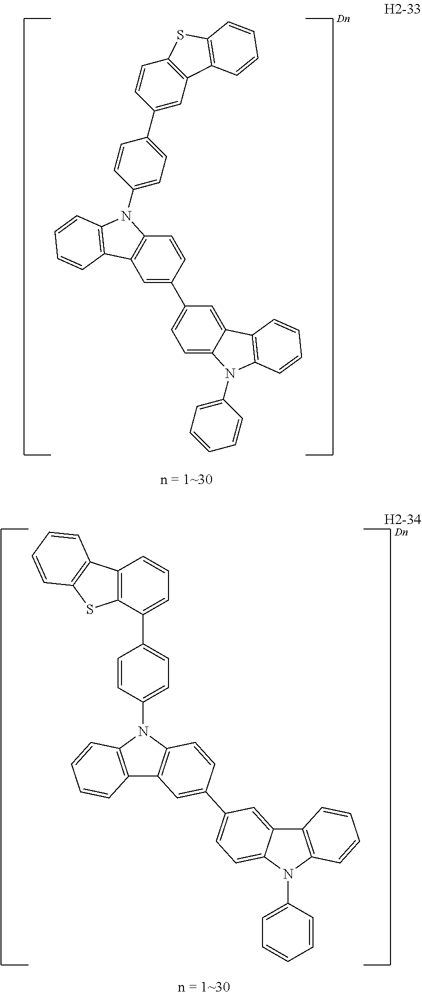 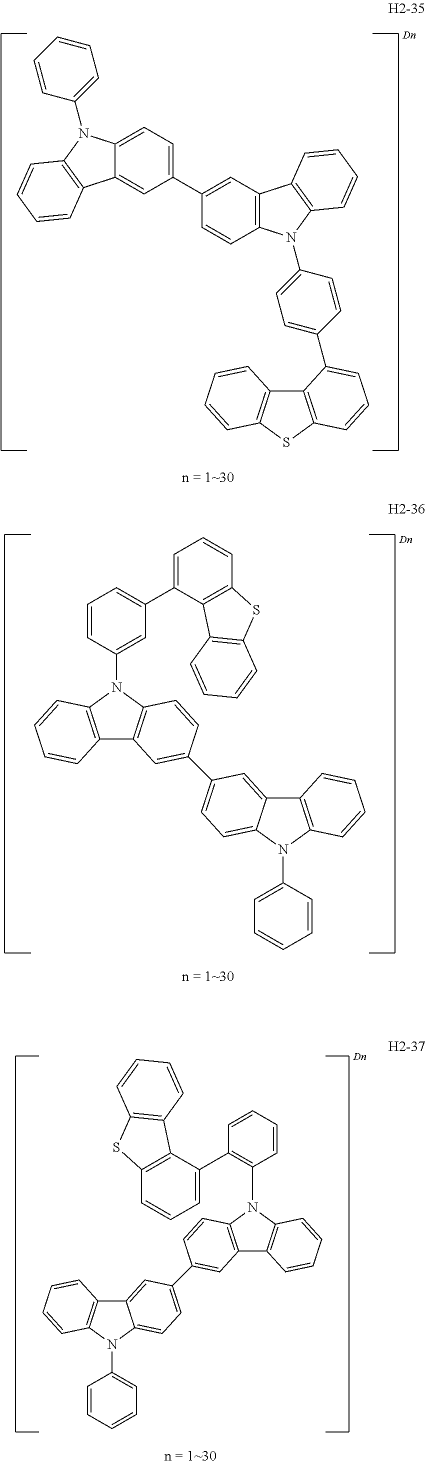 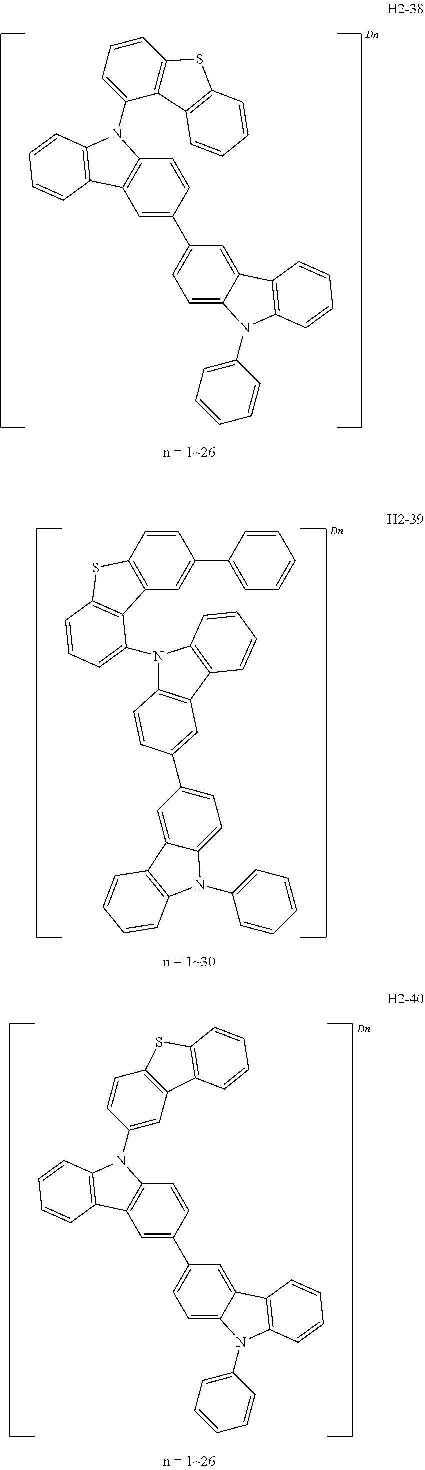 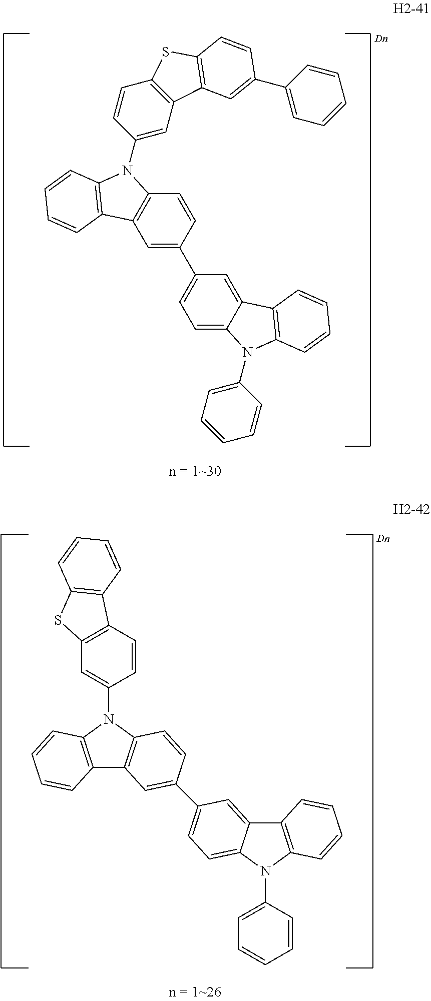 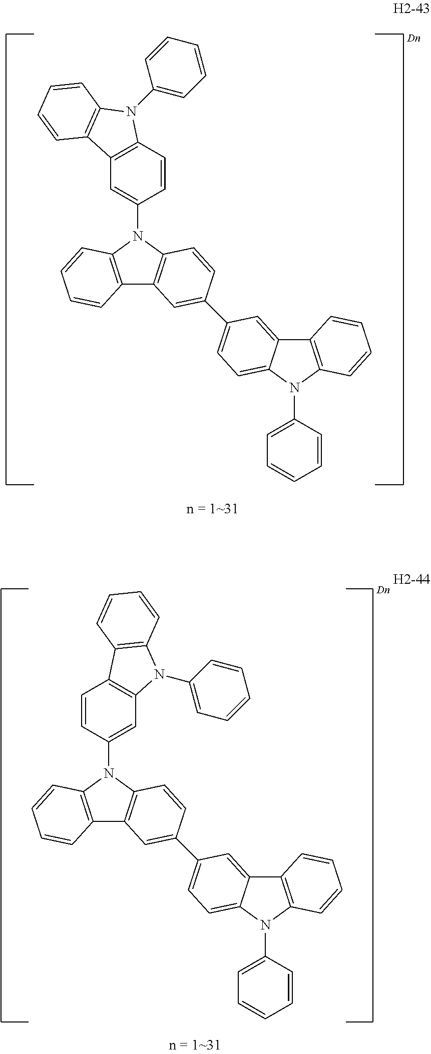 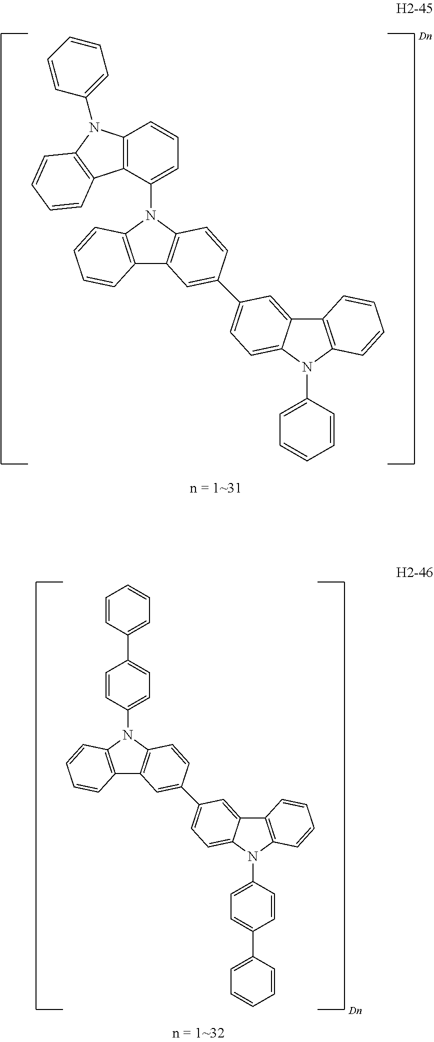 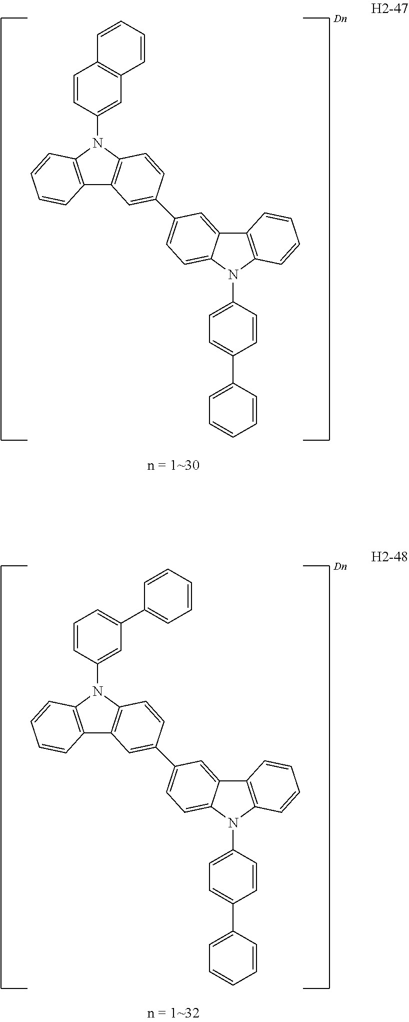 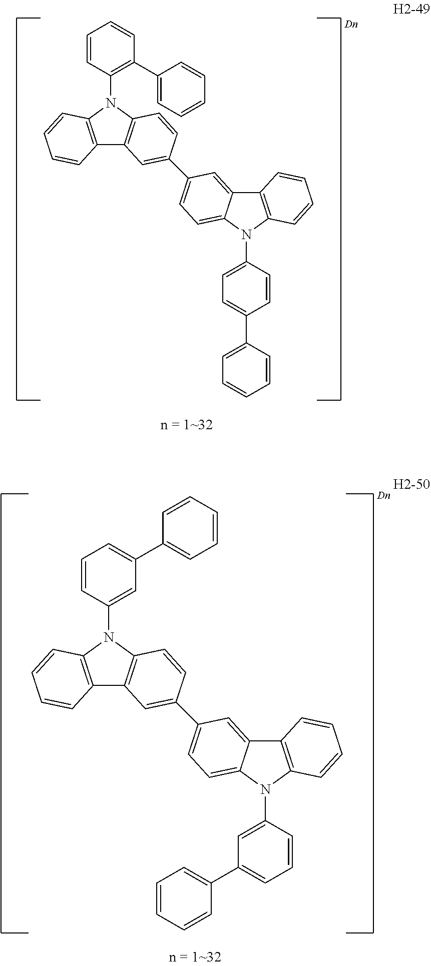 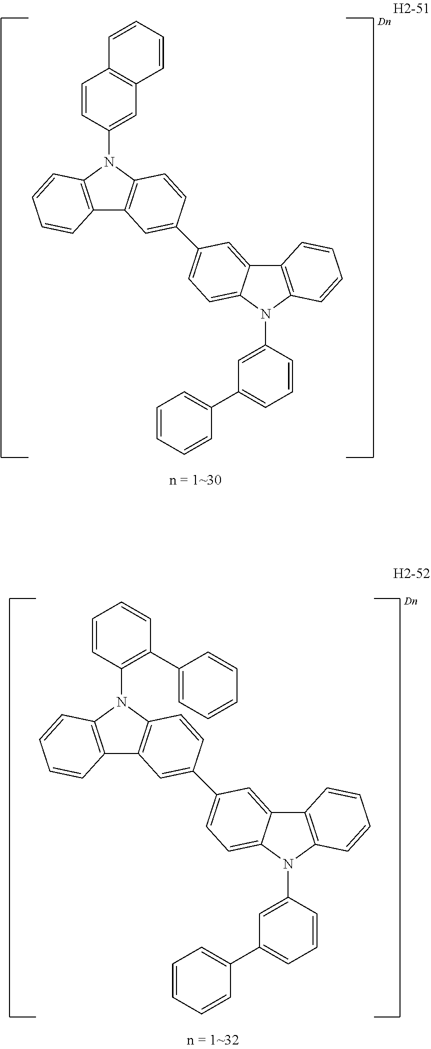 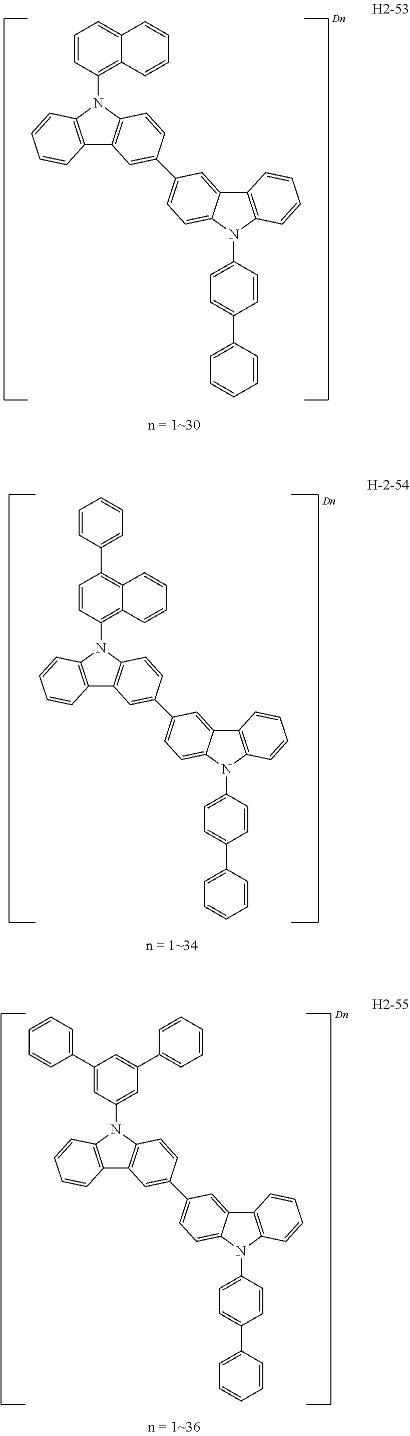 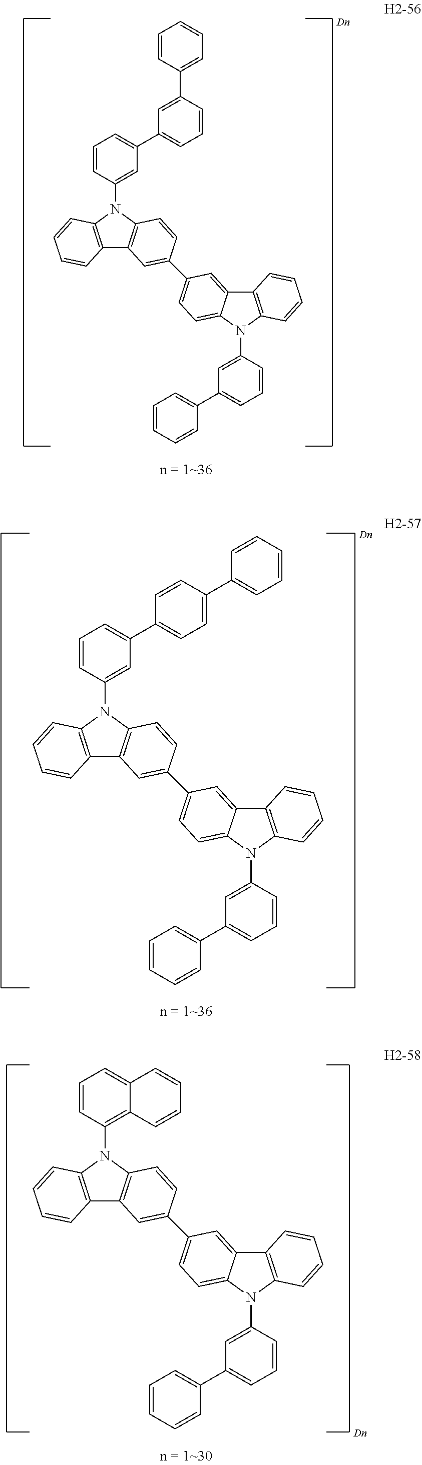 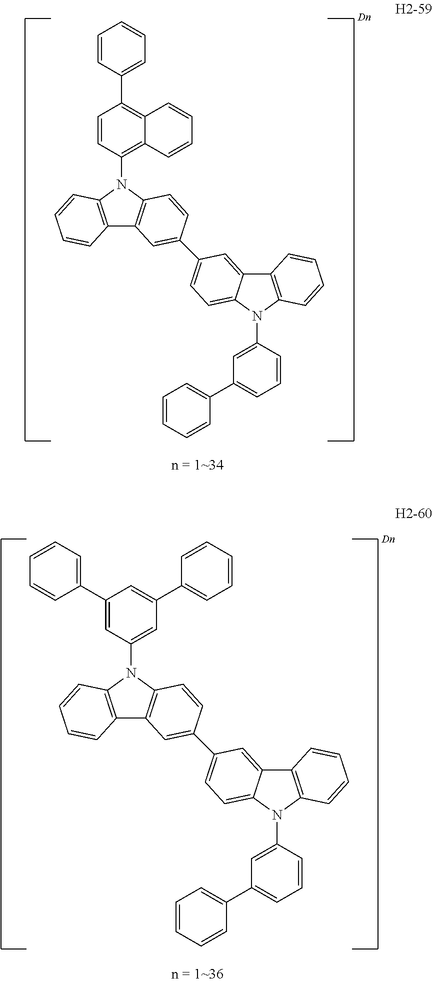 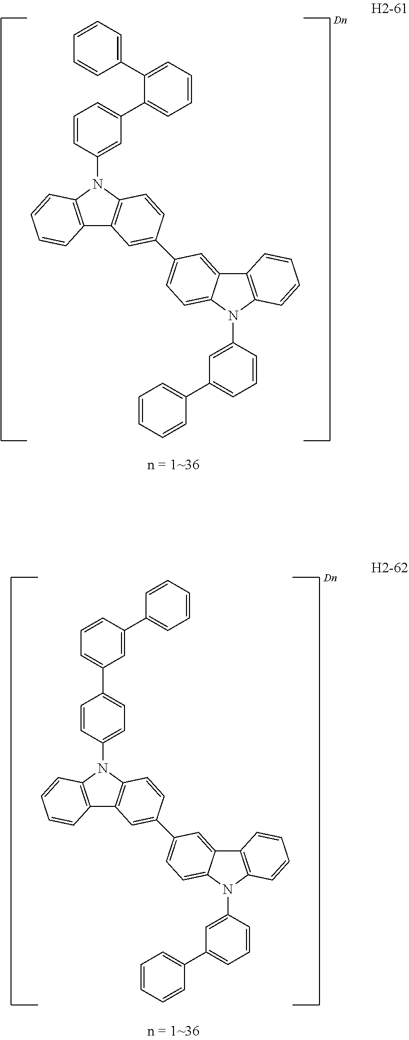 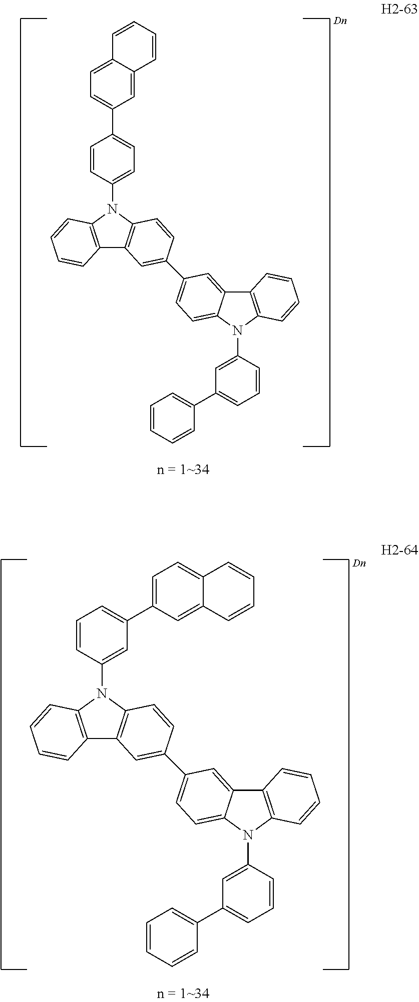 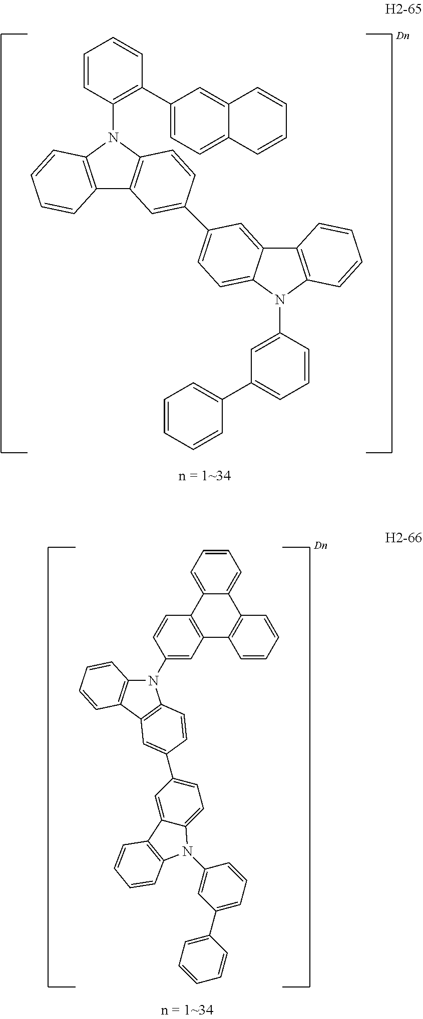 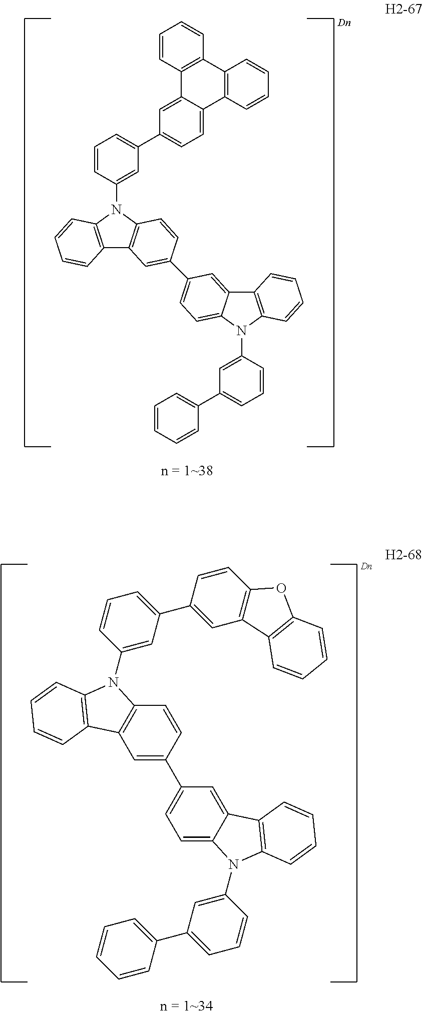 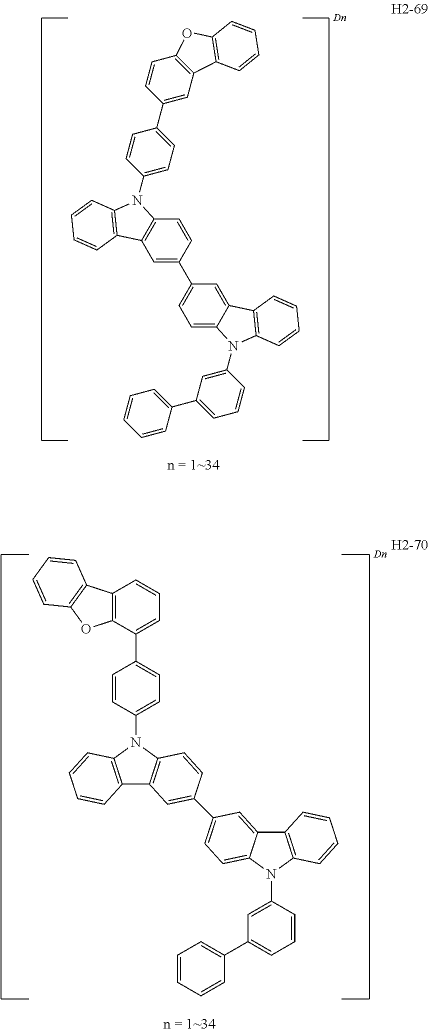 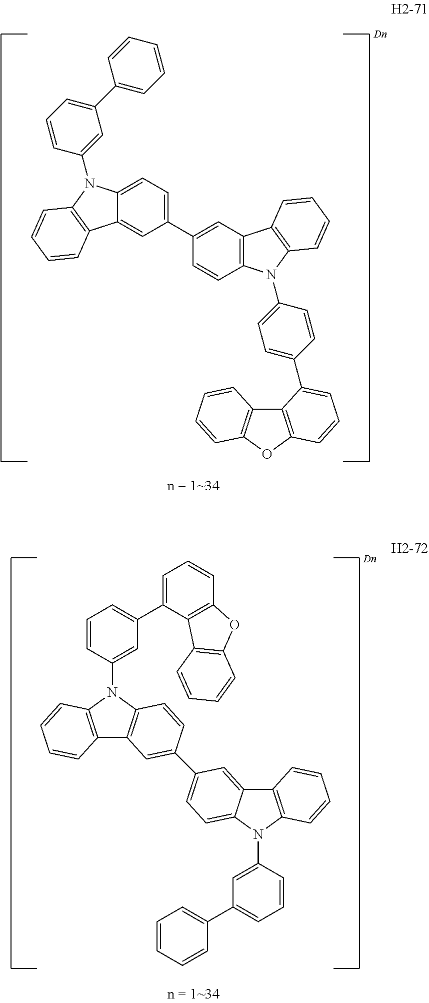 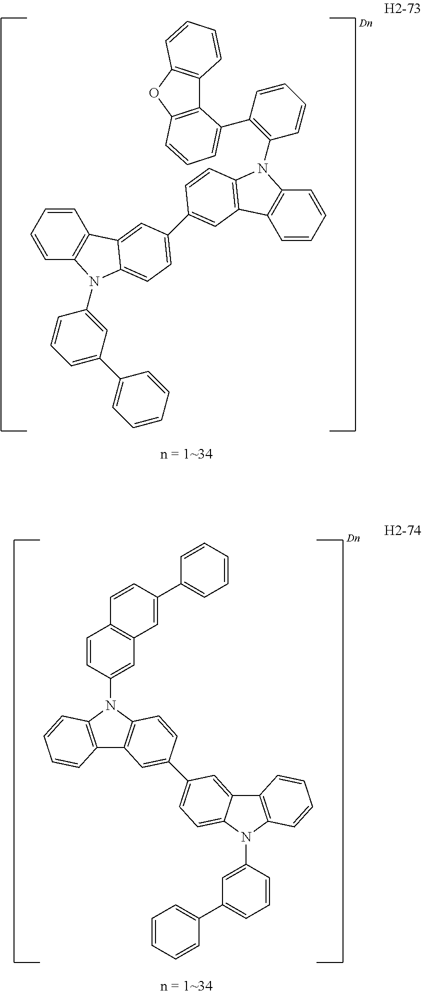 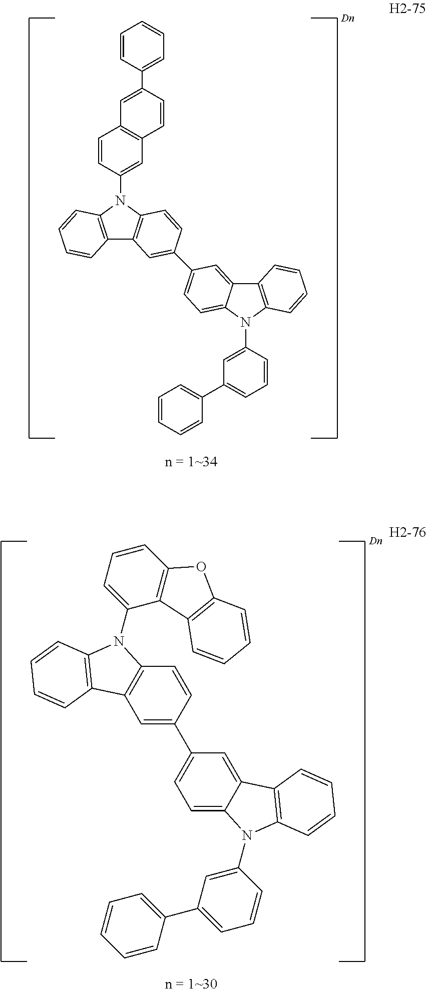 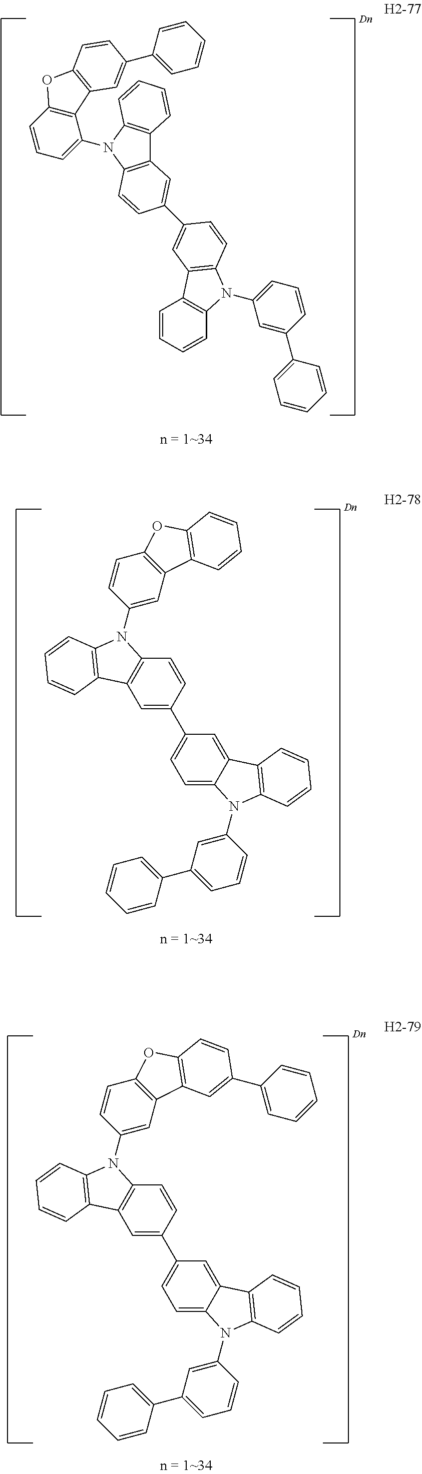 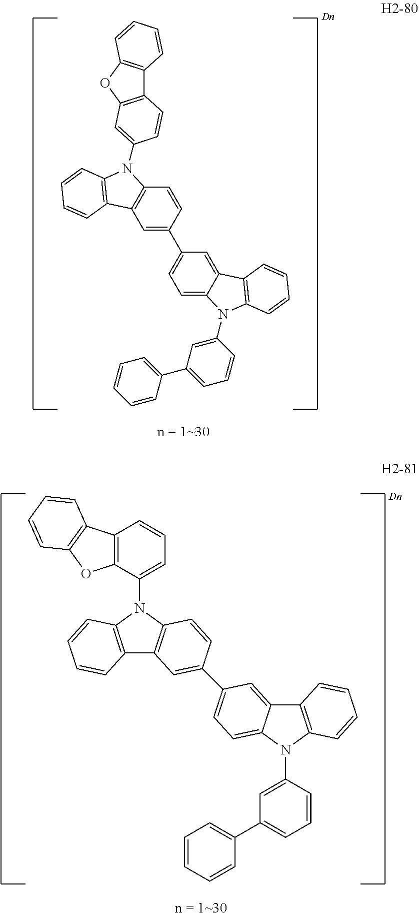 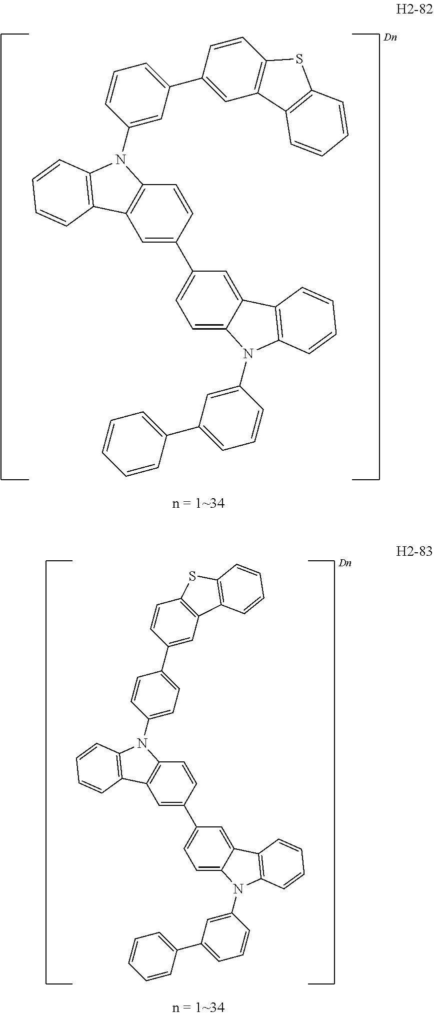 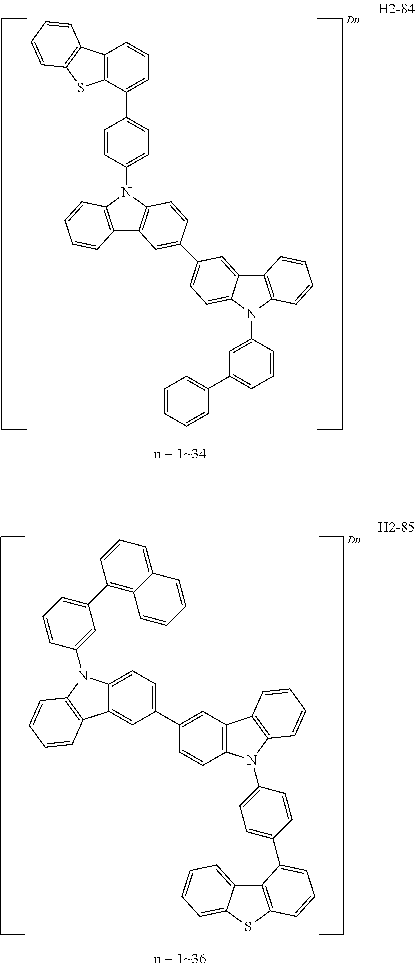 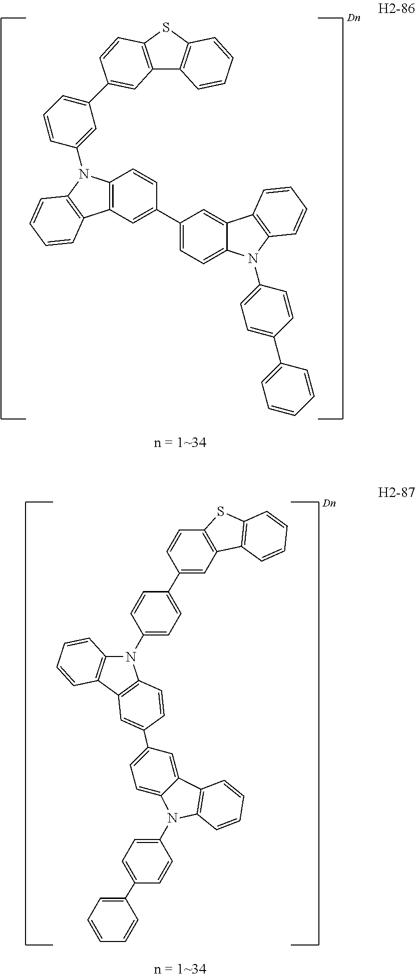 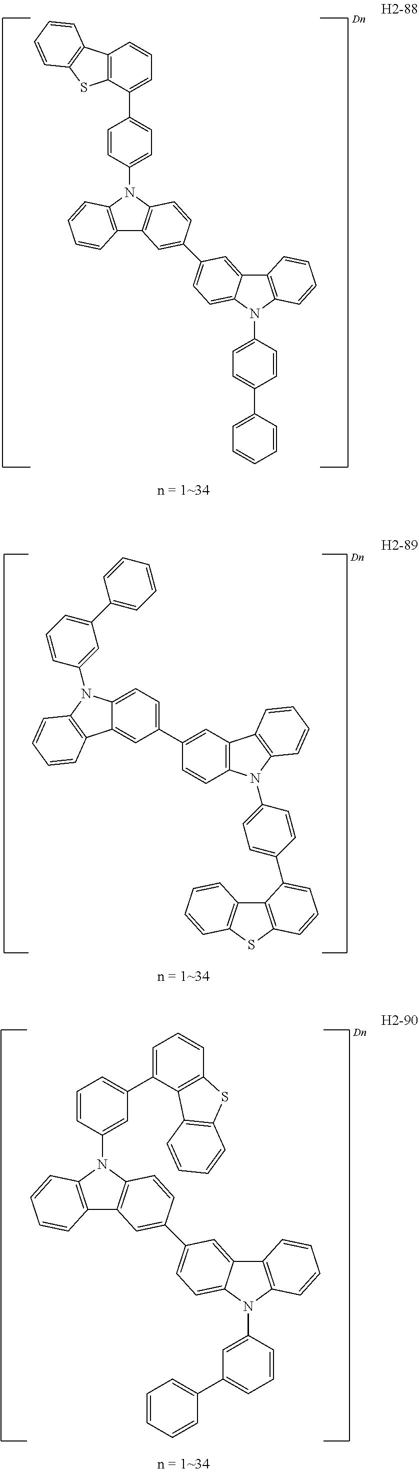 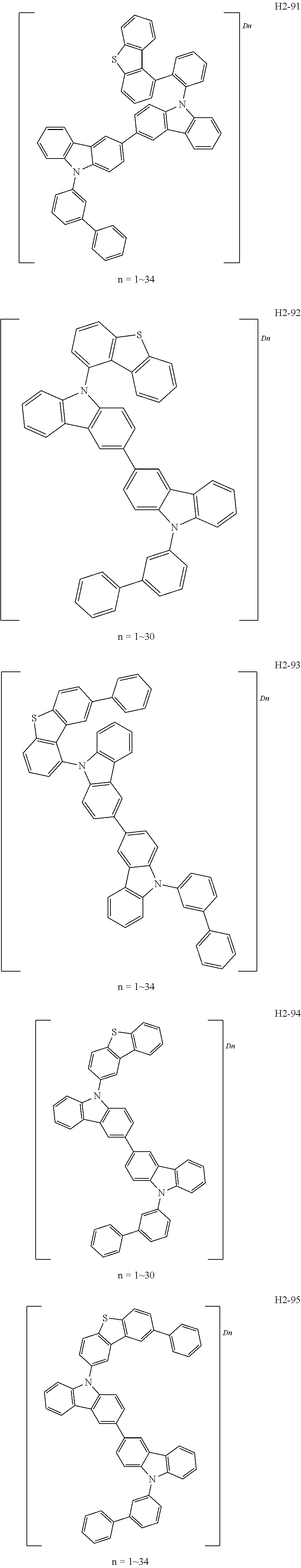 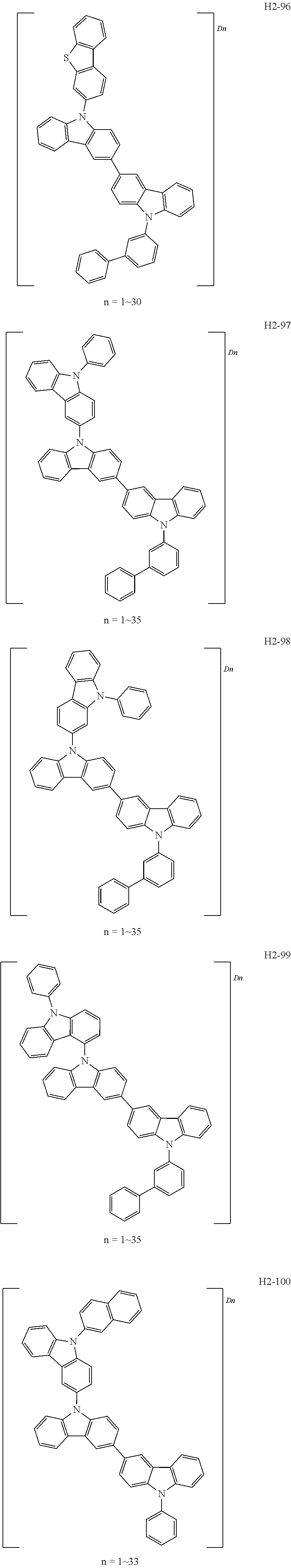 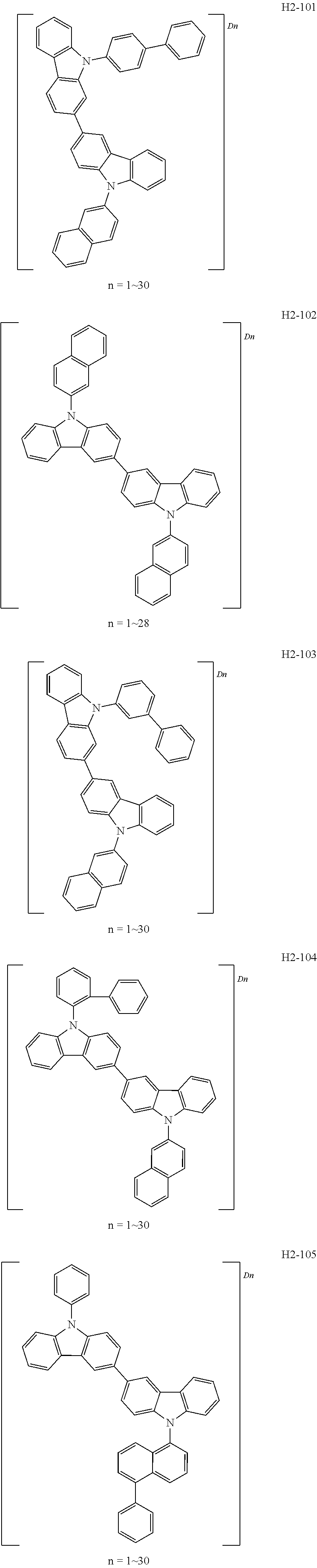 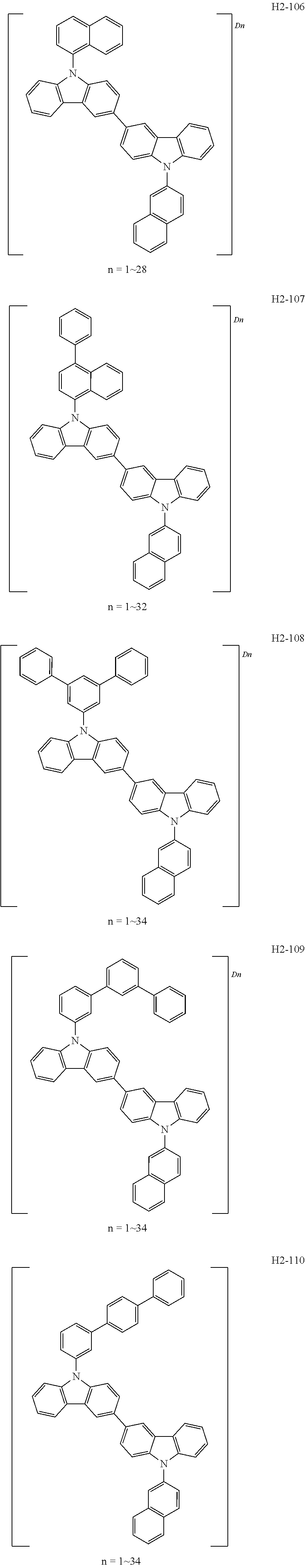 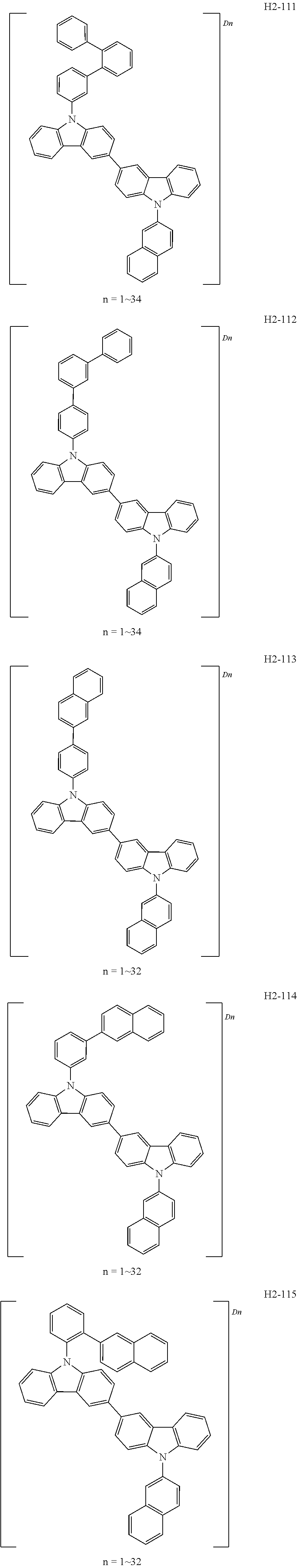 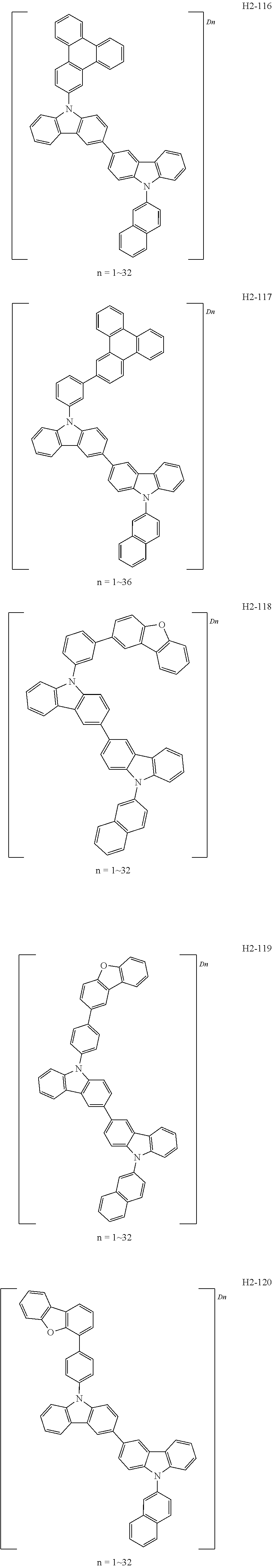 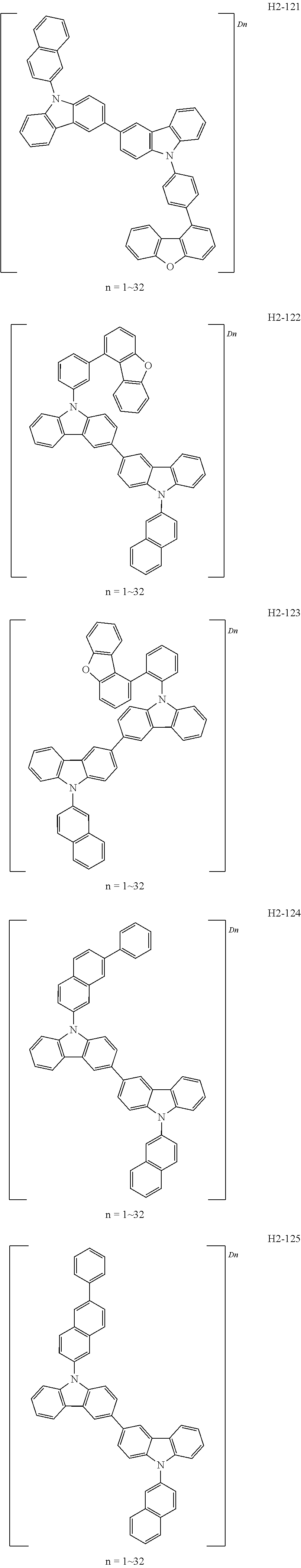 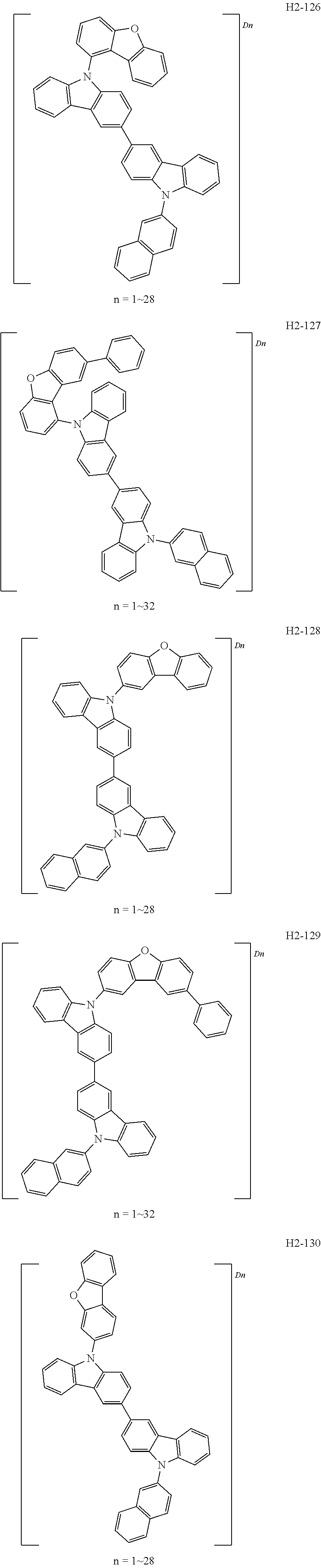 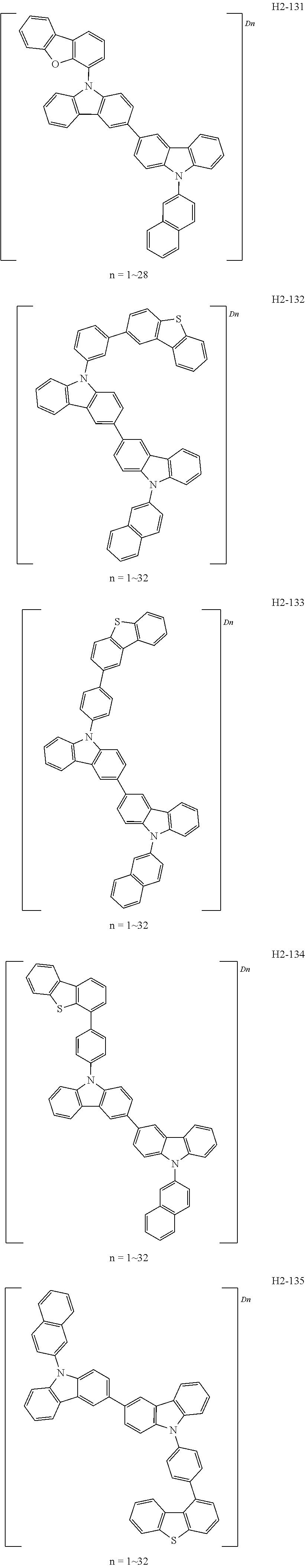 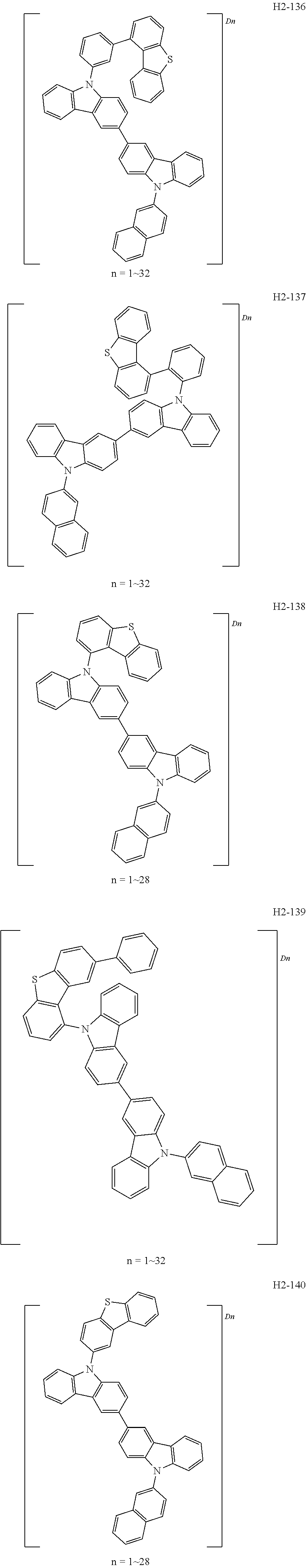 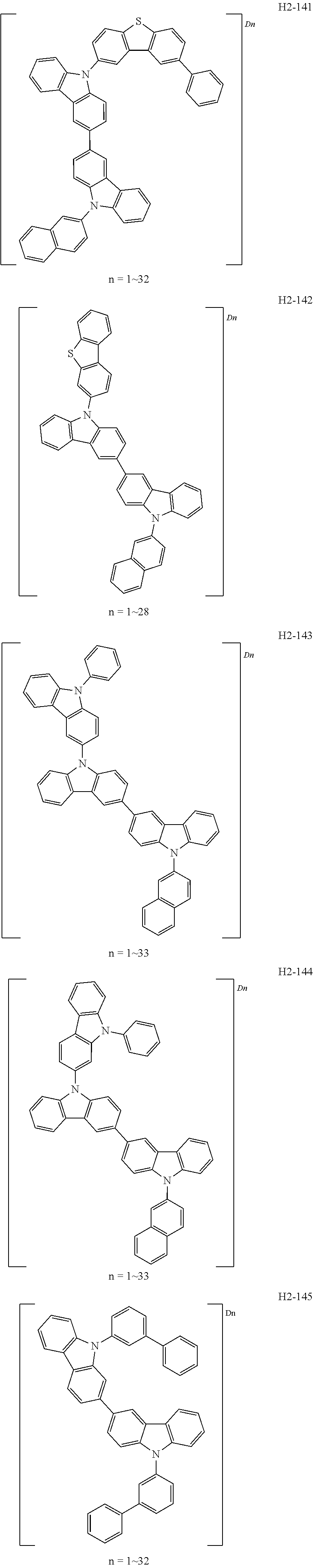 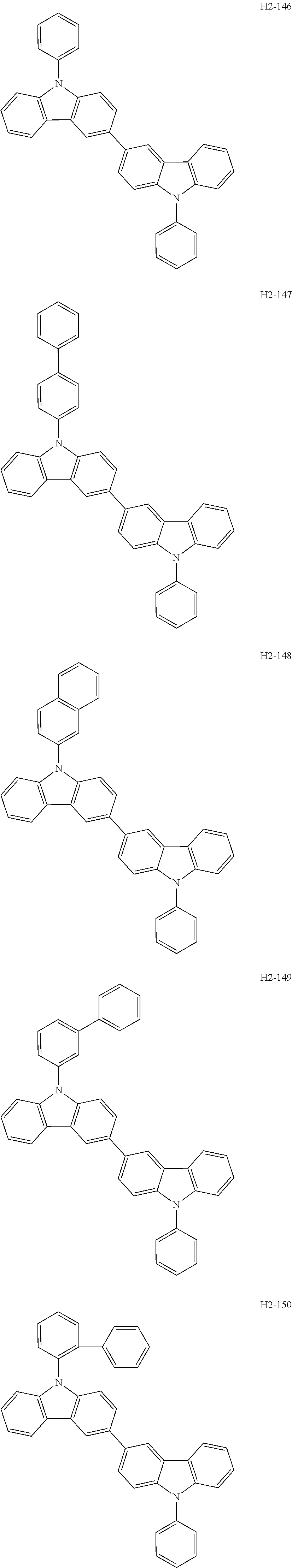 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1] 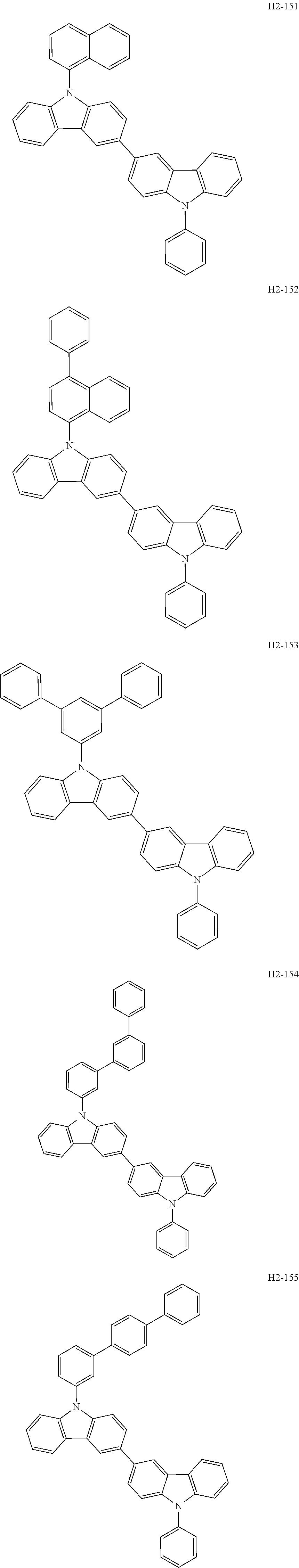 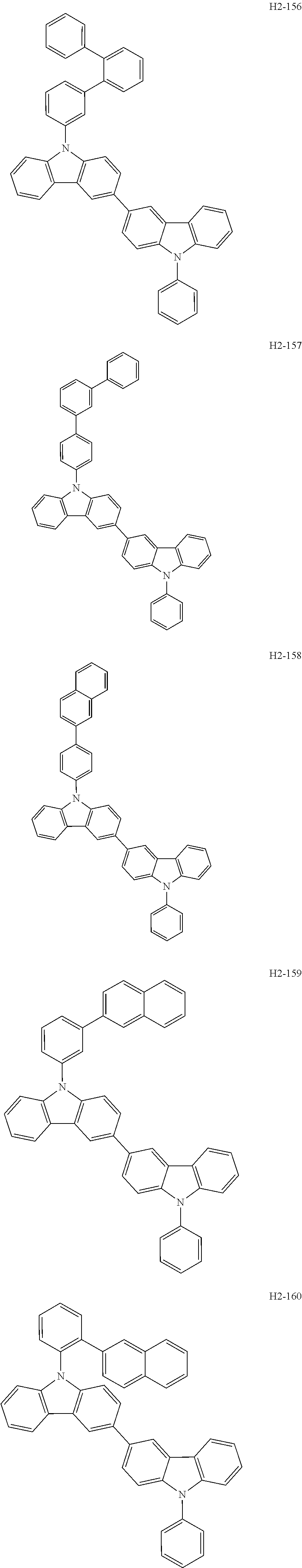 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1] 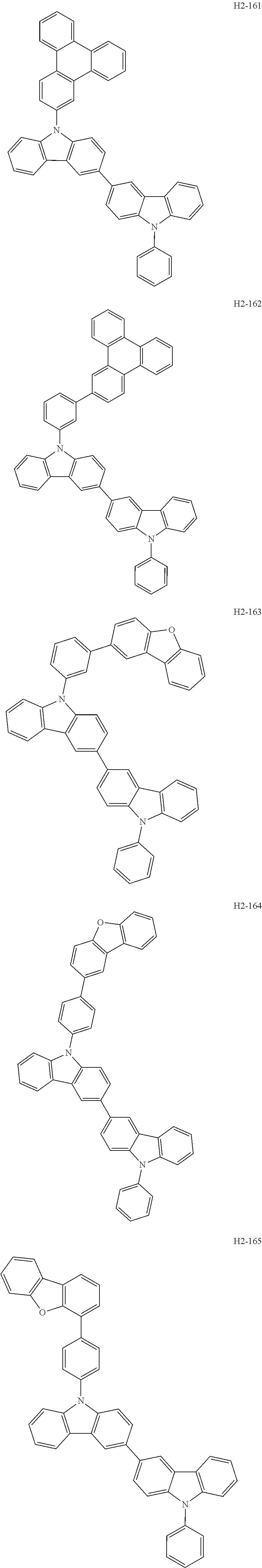 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6c5oc5ccccc56)cc4)ccc32)cc1] 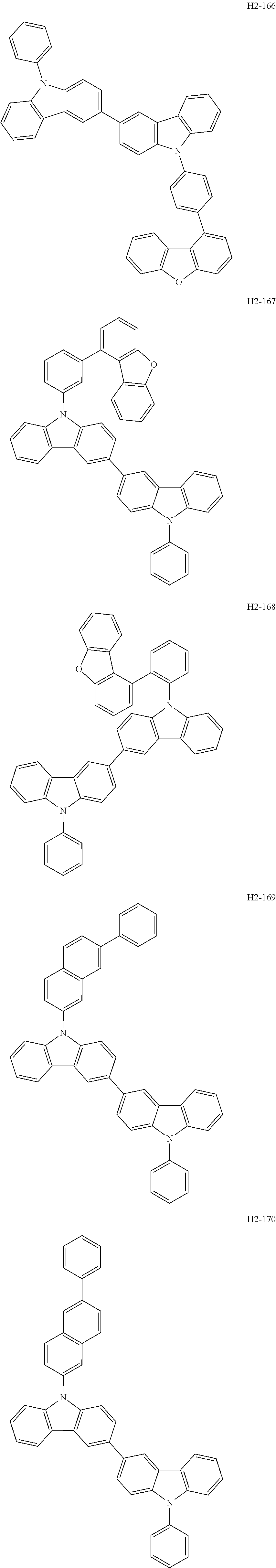 [SMILES: c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1 ; c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1 ; c1ccc(-c2ccc3cc(-n4c5ccccc5c5cc(-c6ccc7c(c6)c6ccccc6n7-c6ccccc6)ccc54)ccc3c2)cc1] 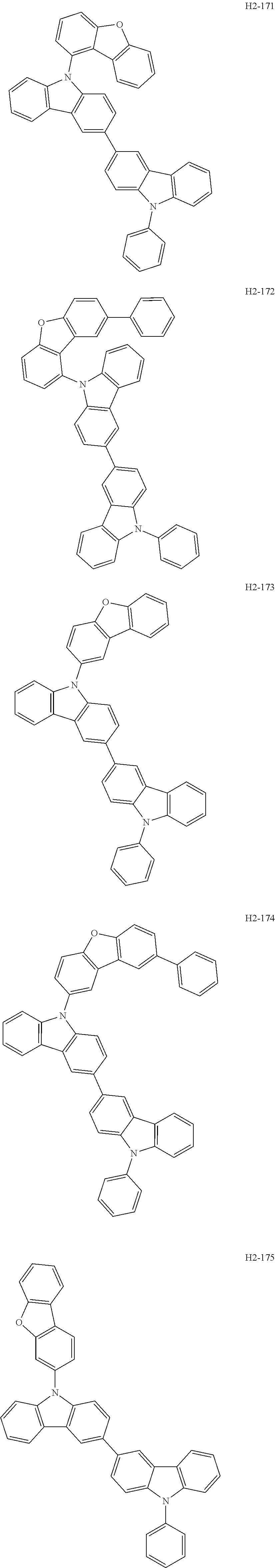 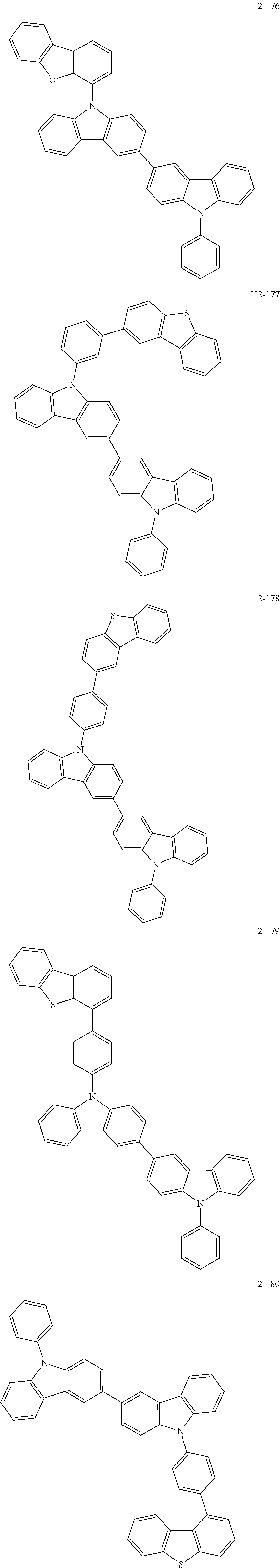 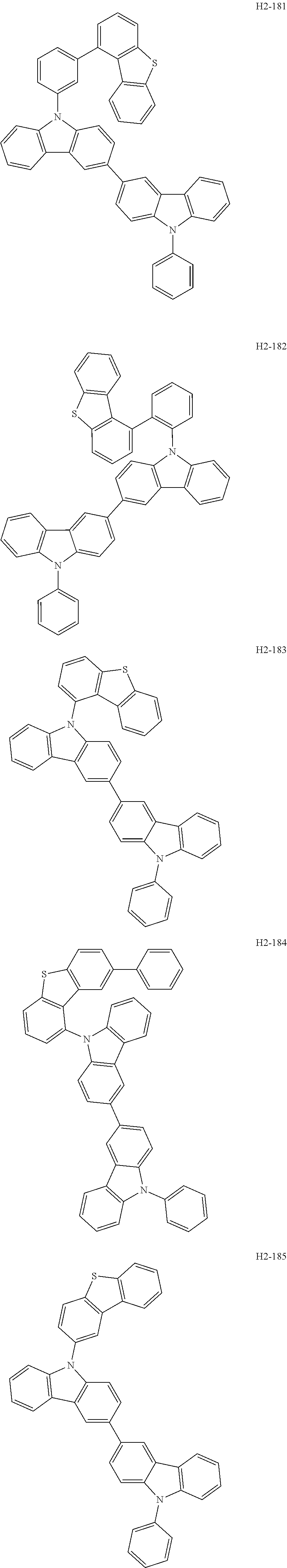 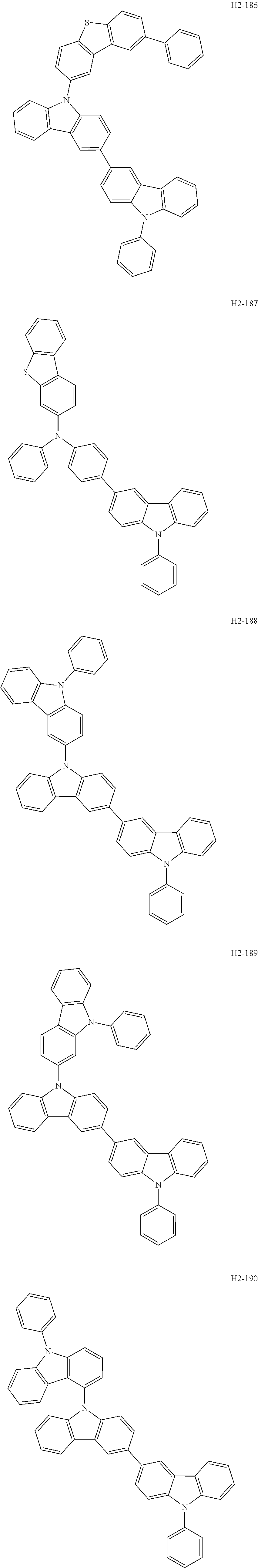 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1] 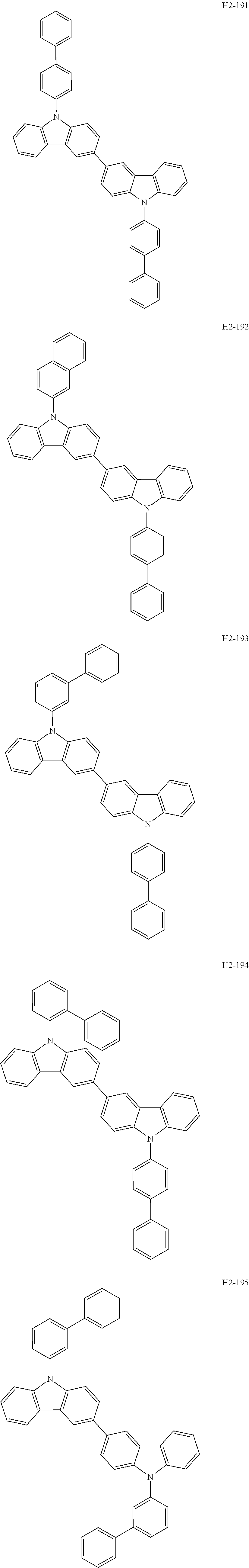 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1] 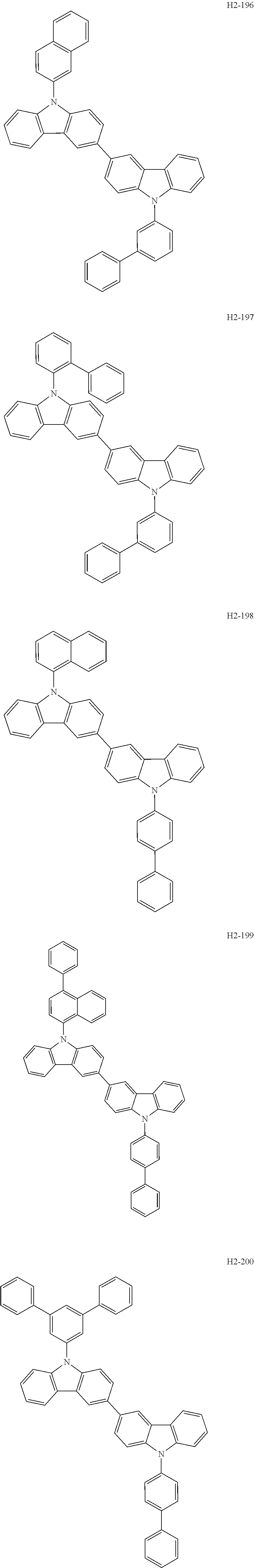 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1] 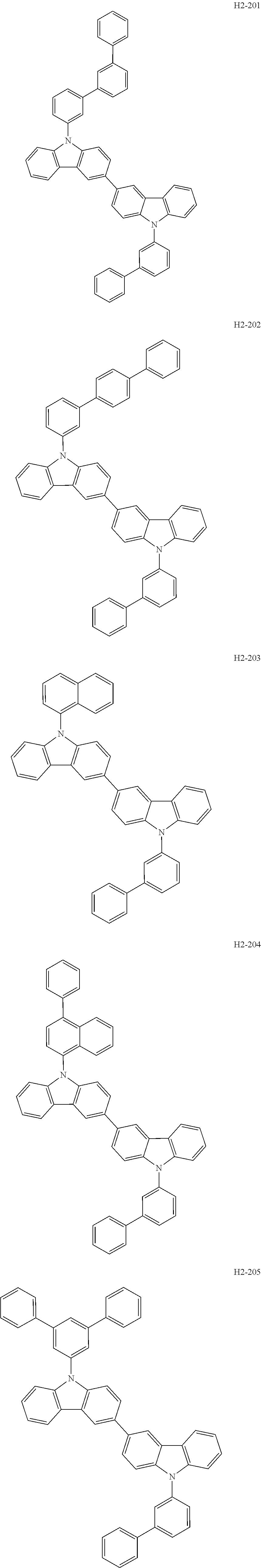 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] 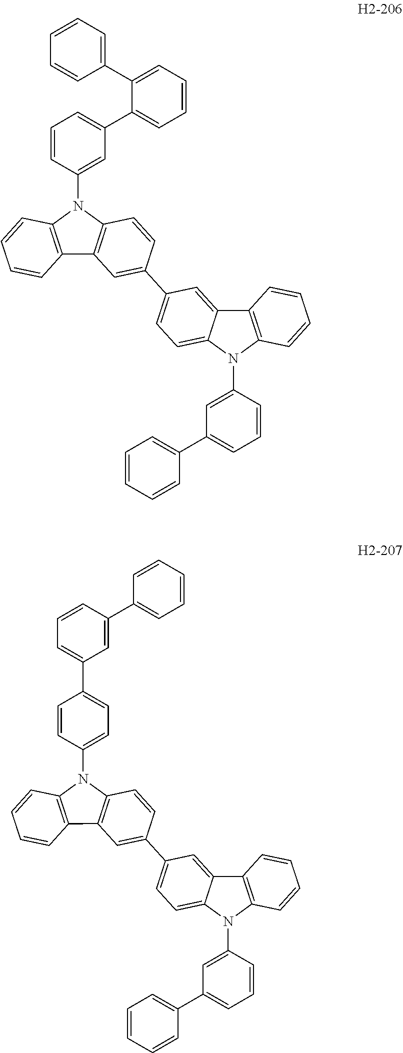 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1] 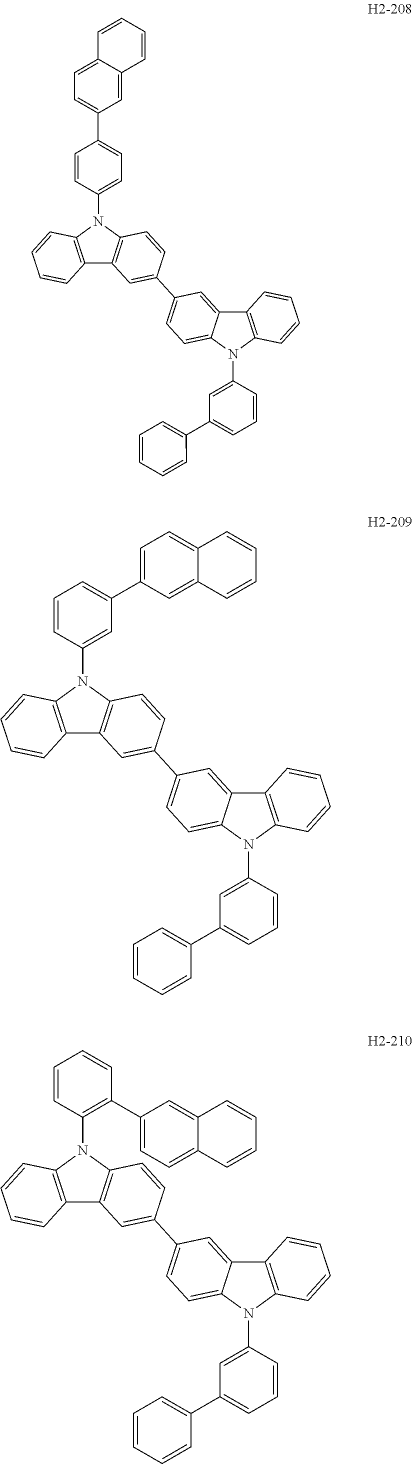 [SMILES: c1ccc(-c2cccc(-n3c4ccccc4c4cc(-c5ccc6c(c5)c5ccccc5n6-c5ccc(-c6ccc7ccccc7c6)cc5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1 ; c1ccc(-c2cccc(-n3c4ccccc4c4cc(-c5ccc6c(c5)c5ccccc5n6-c5ccccc5-c5ccc6ccccc6c5)ccc43)c2)cc1] 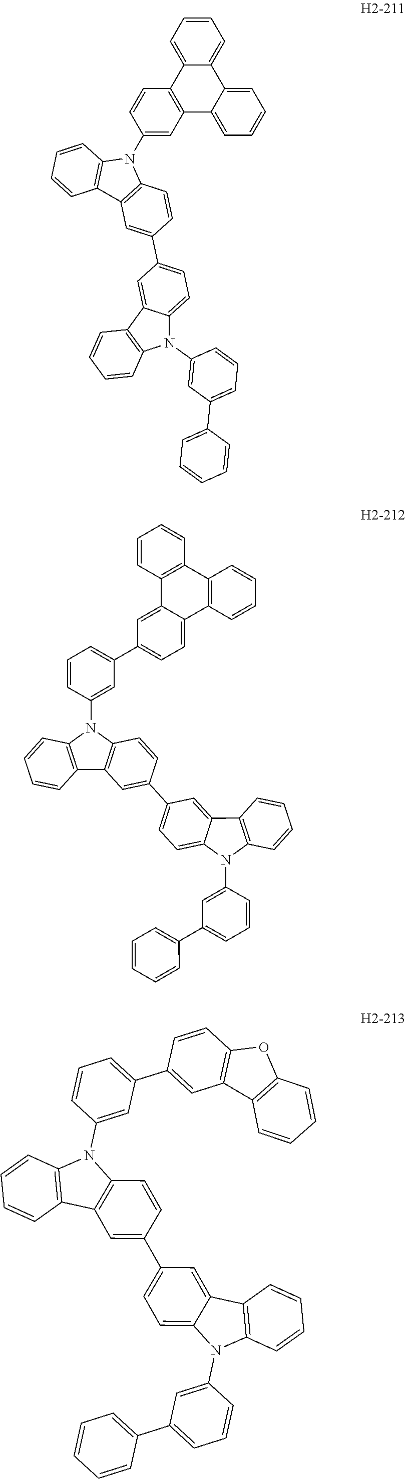 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1 ; c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1] 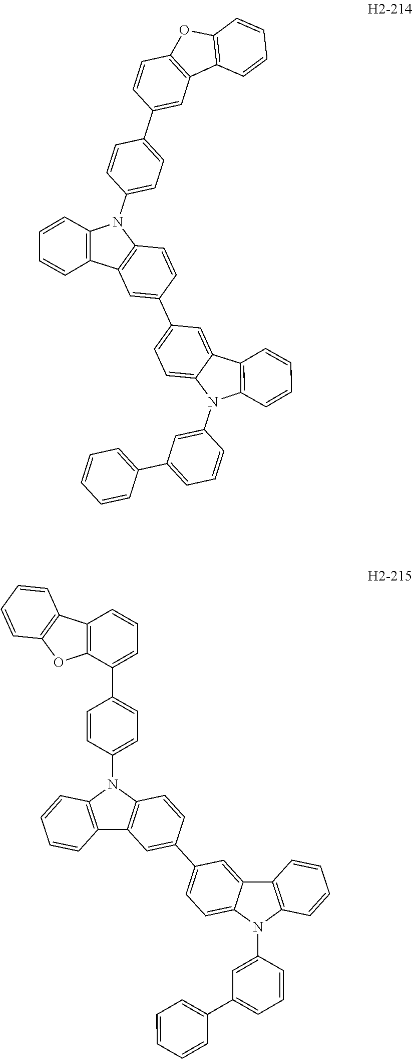 [SMILES: c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1] 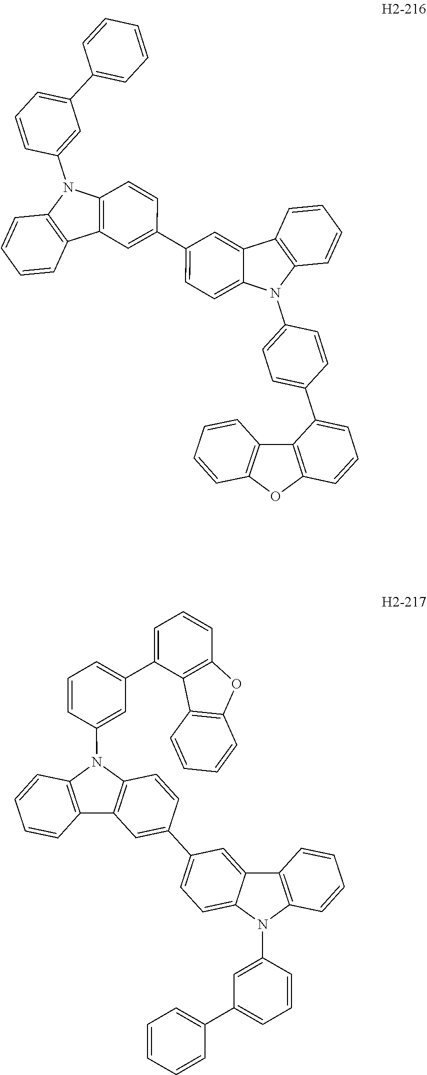 [SMILES: c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1] 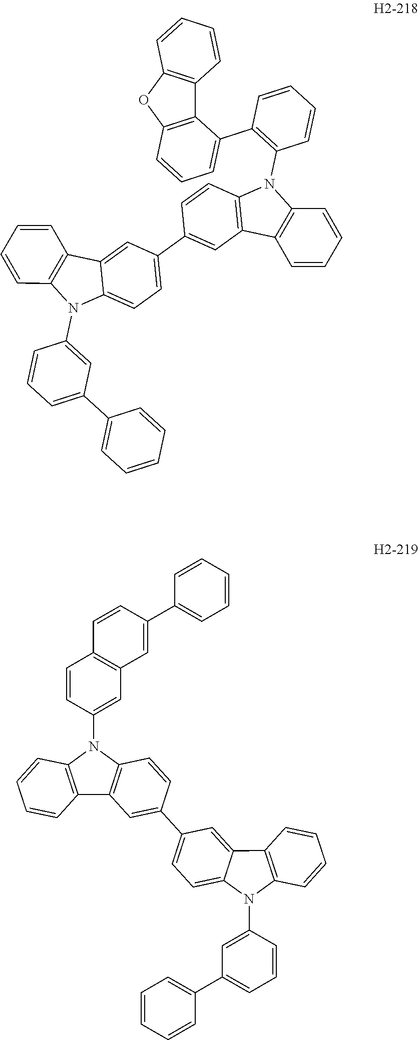 [SMILES: c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1] 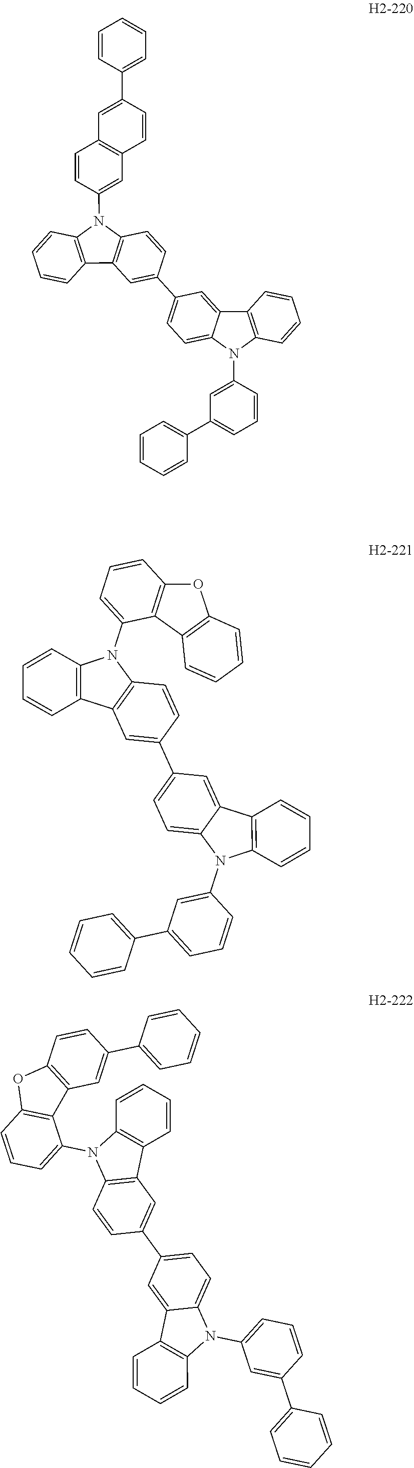 [SMILES: c1ccc(-c2cccc(-n3c4ccccc4c4cc(-c5ccc6c(c5)c5ccccc5n6-c5ccc6cc(-c7ccccc7)ccc6c5)ccc43)c2)cc1 ; c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2cccc(-n3c4ccccc4c4cc(-c5ccc6c(c5)c5ccccc5n6-c5cccc6oc7ccc(-c8ccccc8)cc7c56)ccc43)c2)cc1] 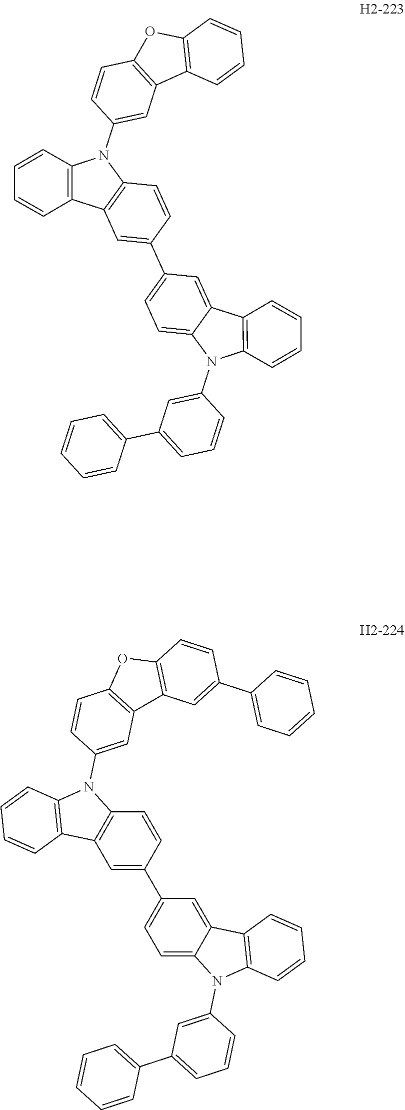 [SMILES: c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1] 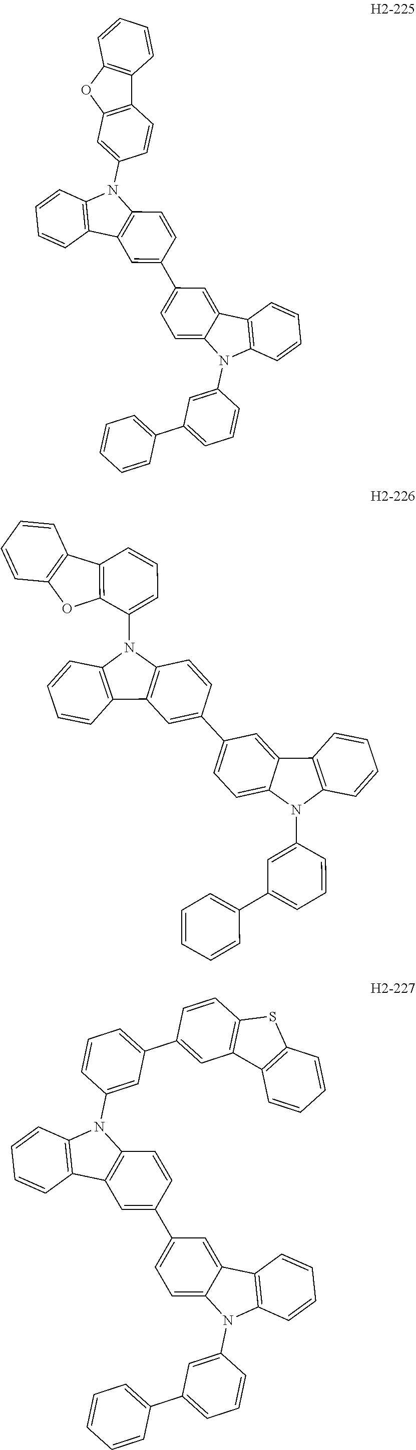 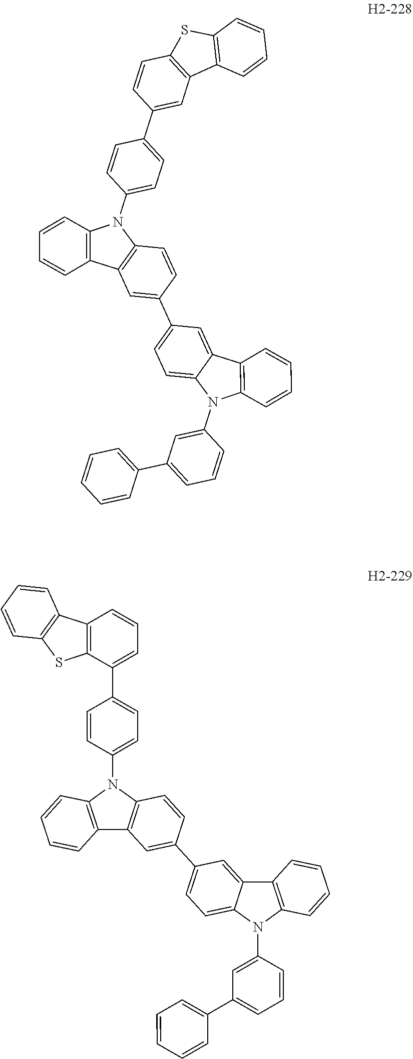 [SMILES: c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1] 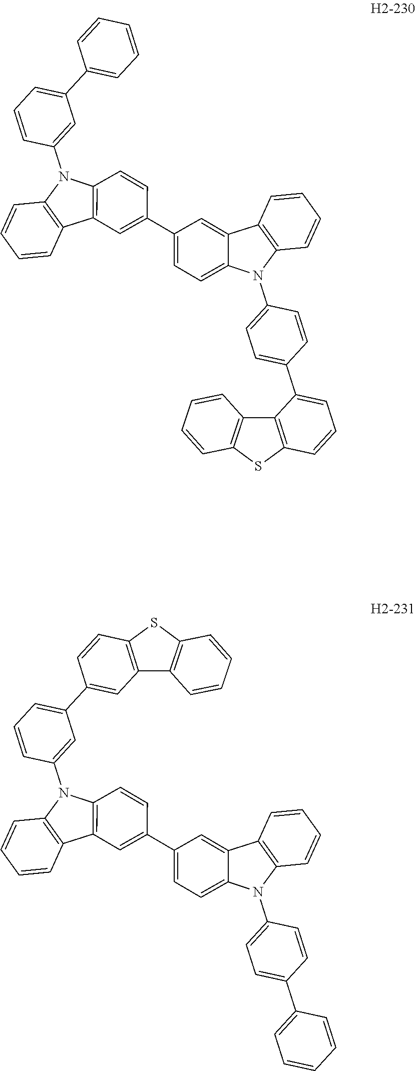 [SMILES: c1ccc(-c2cccc(-n3c4ccccc4c4cc(-c5ccc6c(c5)c5ccccc5n6-c5ccc(-c6cccc7sc8ccccc8c67)cc5)ccc43)c2)cc1 ; c1ccc(-c2ccc(-n3c4ccccc4c4cc(-c5ccc6c(c5)c5ccccc5n6-c5cccc(-c6ccc7sc8ccccc8c7c6)c5)ccc43)cc2)cc1] 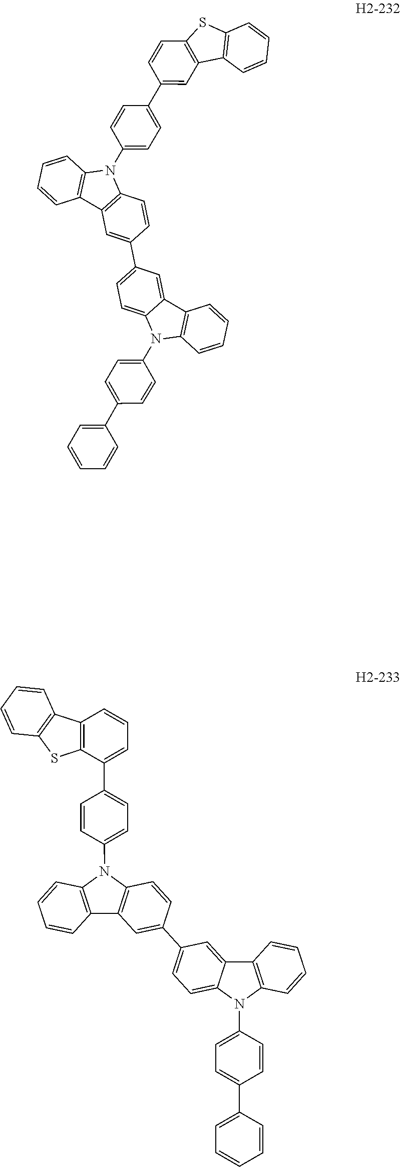 [SMILES: c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1] 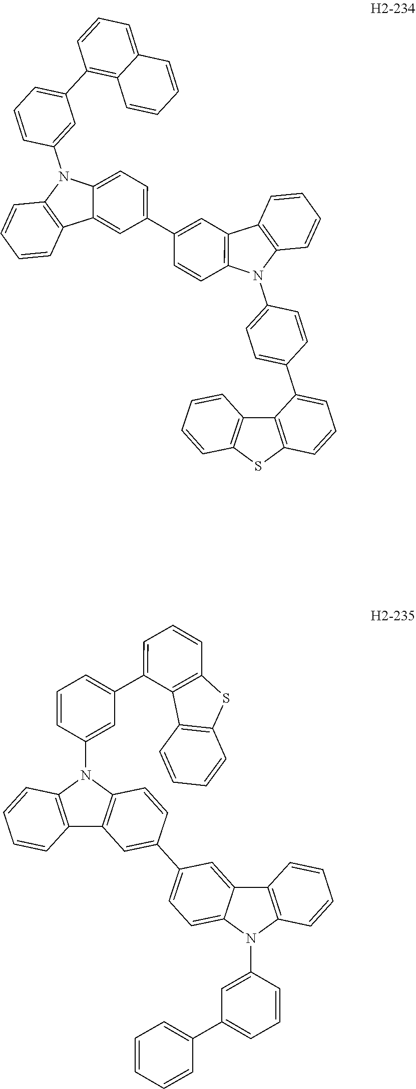 [SMILES: c1cc(-c2cccc3ccccc23)cc(-n2c3ccccc3c3cc(-c4ccc5c(c4)c4ccccc4n5-c4ccc(-c5cccc6sc7ccccc7c56)cc4)ccc32)c1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1] 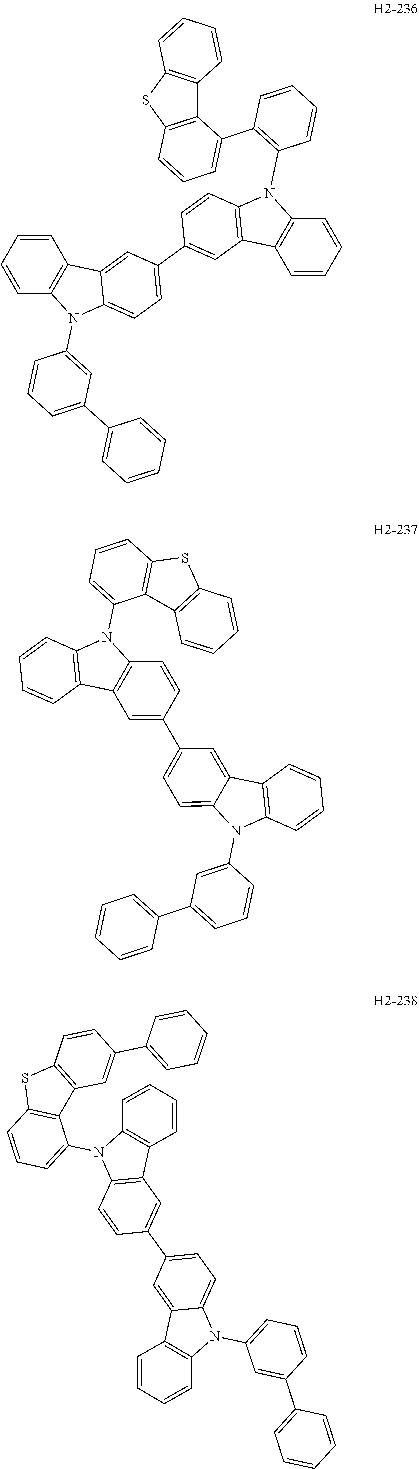 [SMILES: c1ccc(-c2cccc(-n3c4ccccc4c4cc(-c5ccc6c(c5)c5ccccc5n6-c5ccccc5-c5cccc6sc7ccccc7c56)ccc43)c2)cc1 ; c1ccc(-c2cccc(-n3c4ccccc4c4cc(-c5ccc6c(c5)c5ccccc5n6-c5cccc6sc7ccccc7c56)ccc43)c2)cc1 ; c1ccc(-c2cccc(-n3c4ccccc4c4cc(-c5ccc6c(c5)c5ccccc5n6-c5cccc6sc7ccc(-c8ccccc8)cc7c56)ccc43)c2)cc1] 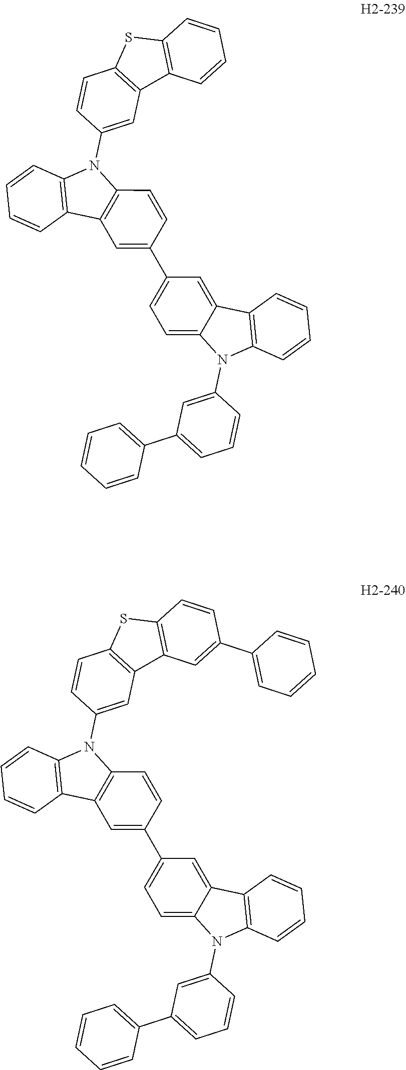 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-c2cccc(-n3c4ccccc4c4cc(-c5ccc6c(c5)c5ccccc5n6-c5ccc6sc7ccc(-c8ccccc8)cc7c6c5)ccc43)c2)cc1] 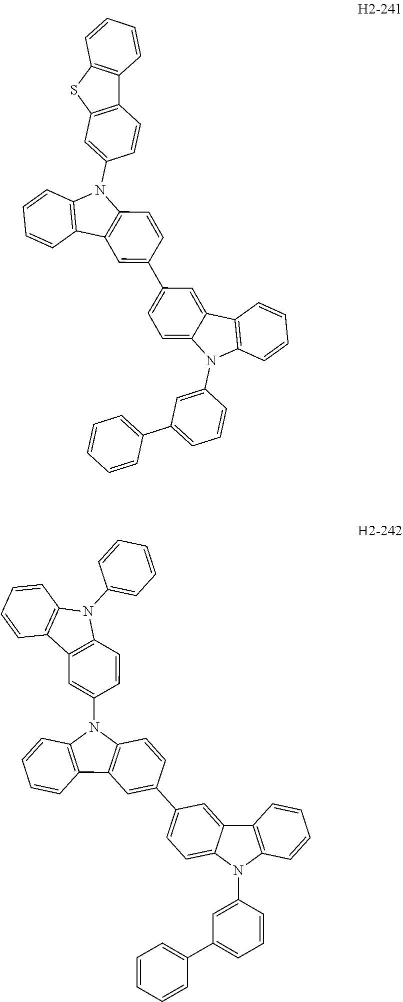 [SMILES: c1ccc(-c2cccc(-n3c4ccccc4c4cc(-c5ccc6c(c5)c5ccccc5n6-c5ccc6c(c5)sc5ccccc56)ccc43)c2)cc1 ; c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1] 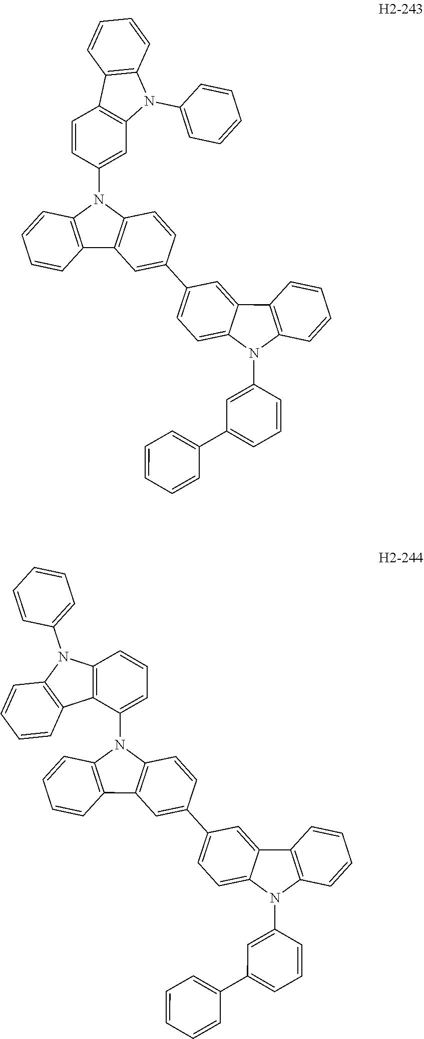 [SMILES: c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1 ; c1ccc(-c2cccc(-n3c4ccccc4c4cc(-c5ccc6c(c5)c5ccccc5n6-c5cccc6c5c5ccccc5n6-c5ccccc5)ccc43)c2)cc1] 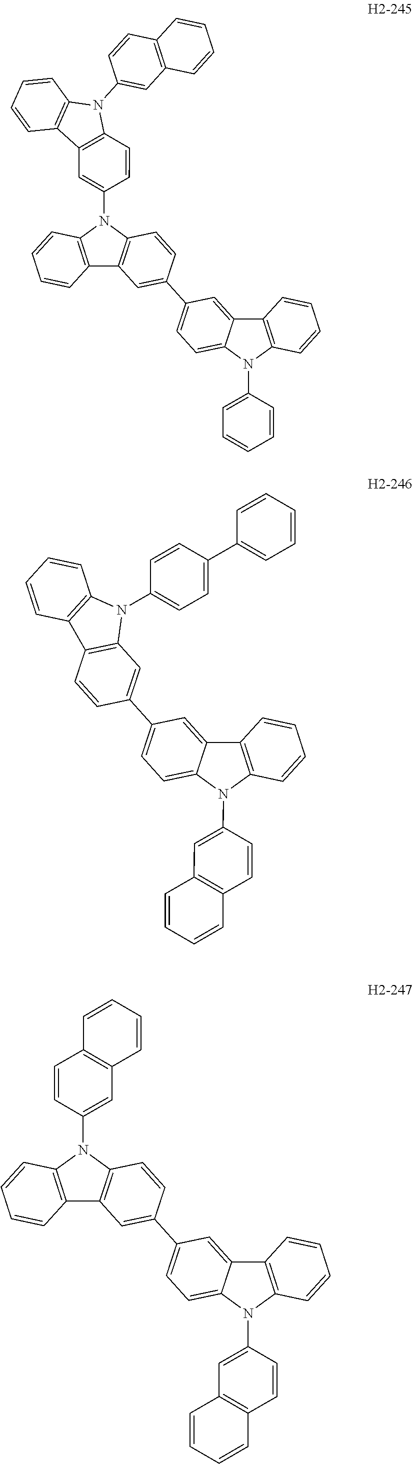 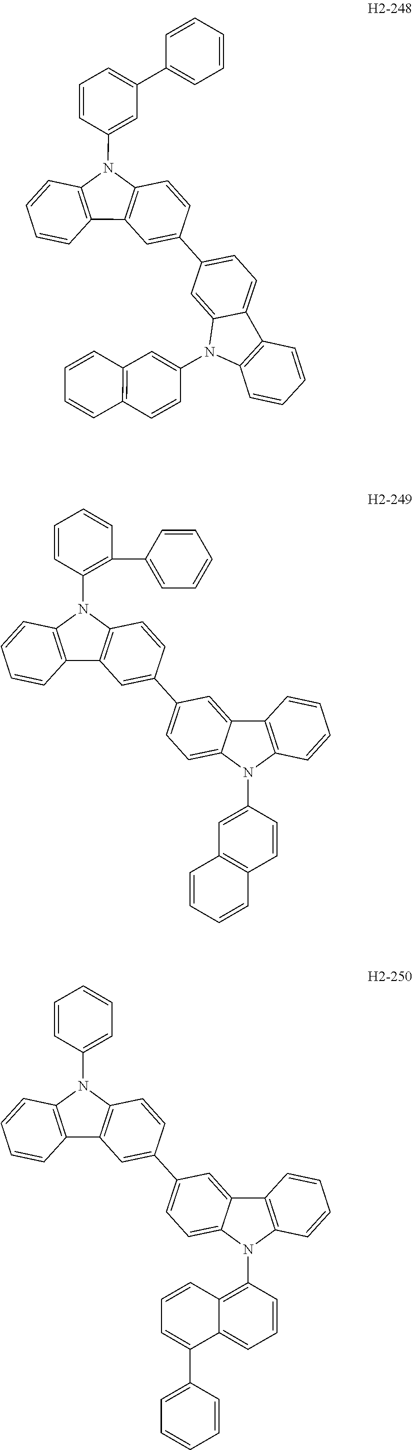 [SMILES: c1ccc(-c2cccc(-n3c4ccccc4c4cc(-c5ccc6c7ccccc7n(-c7ccc8ccccc8c7)c6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1] 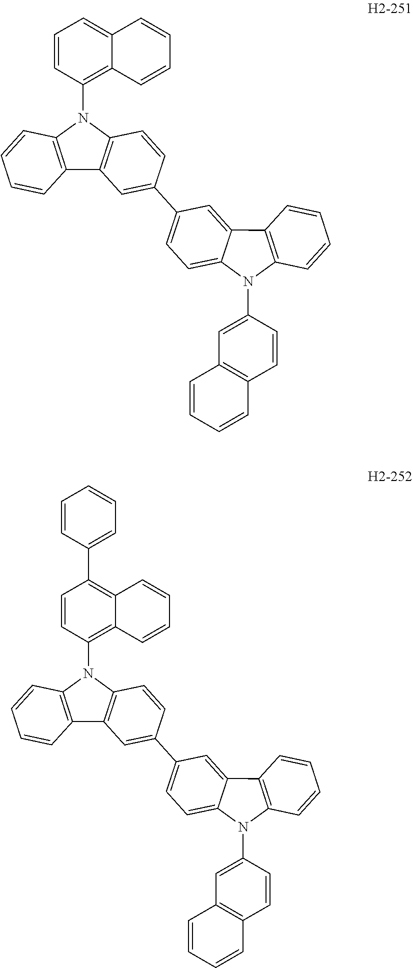 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1] 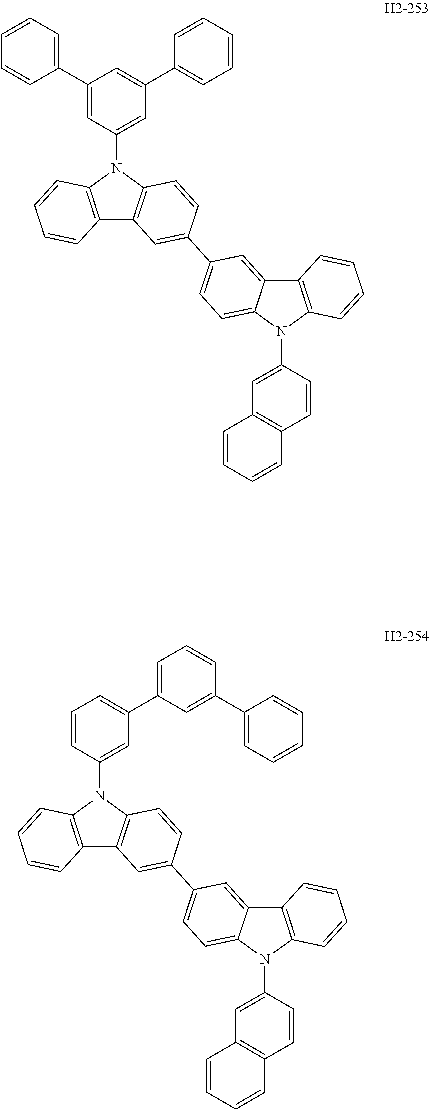 [SMILES: c1ccc(-c2cc(-c3ccccc3)cc(-n3c4ccccc4c4cc(-c5ccc6c(c5)c5ccccc5n6-c5ccc6ccccc6c5)ccc43)c2)cc1 ; c1ccc(-c2cccc(-c3cccc(-n4c5ccccc5c5cc(-c6ccc7c(c6)c6ccccc6n7-c6ccc7ccccc7c6)ccc54)c3)c2)cc1] 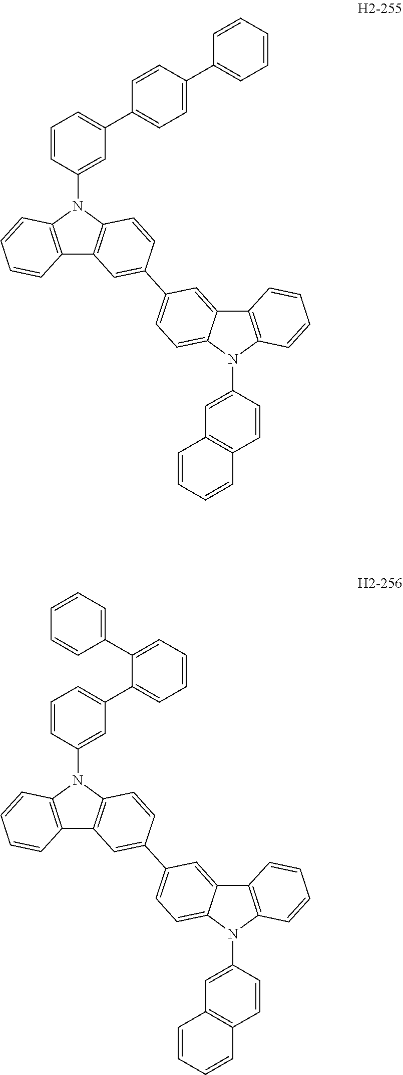 [SMILES: c1ccc(-c2ccc(-c3cccc(-n4c5ccccc5c5cc(-c6ccc7c(c6)c6ccccc6n7-c6ccc7ccccc7c6)ccc54)c3)cc2)cc1 ; c1ccc(-c2ccccc2-c2cccc(-n3c4ccccc4c4cc(-c5ccc6c(c5)c5ccccc5n6-c5ccc6ccccc6c5)ccc43)c2)cc1] 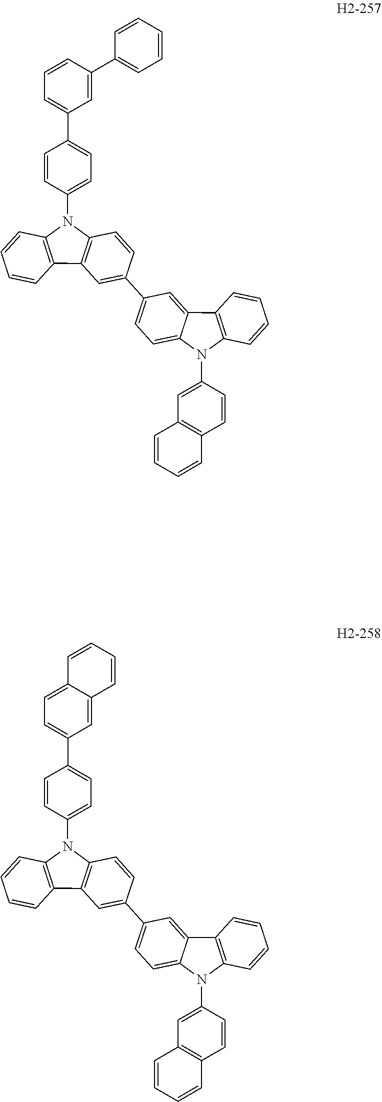 [SMILES: c1ccc(-c2cccc(-c3ccc(-n4c5ccccc5c5cc(-c6ccc7c(c6)c6ccccc6n7-c6ccc7ccccc7c6)ccc54)cc3)c2)cc1 ; c1ccc2cc(-c3ccc(-n4c5ccccc5c5cc(-c6ccc7c(c6)c6ccccc6n7-c6ccc7ccccc7c6)ccc54)cc3)ccc2c1] 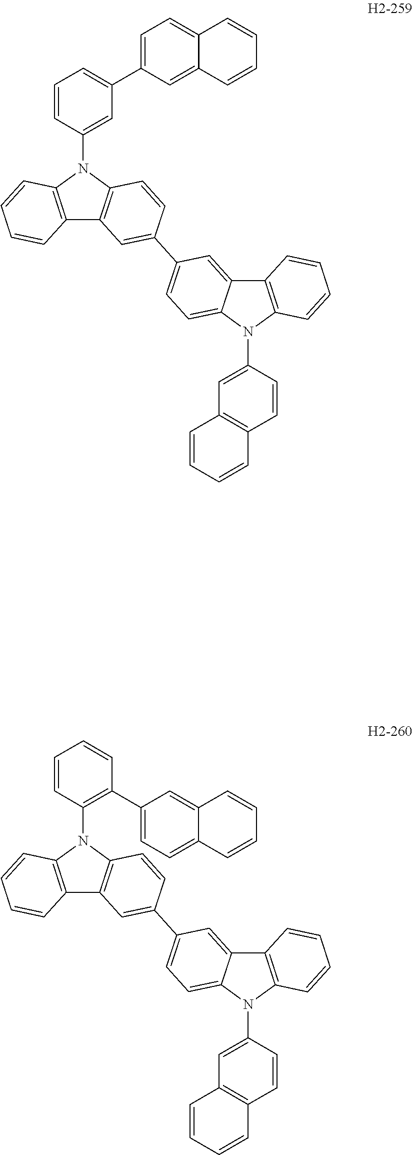 [SMILES: c1cc(-c2ccc3ccccc3c2)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2ccc3ccccc3c2)c1] 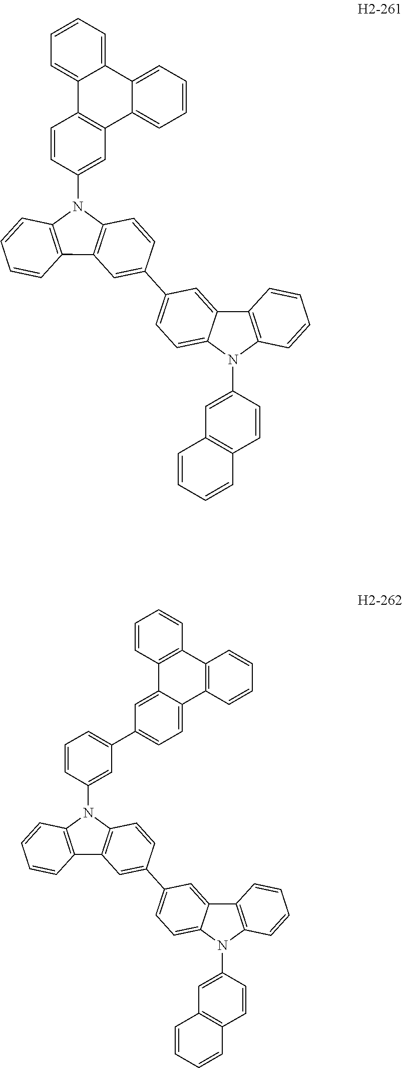 [SMILES: c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1] 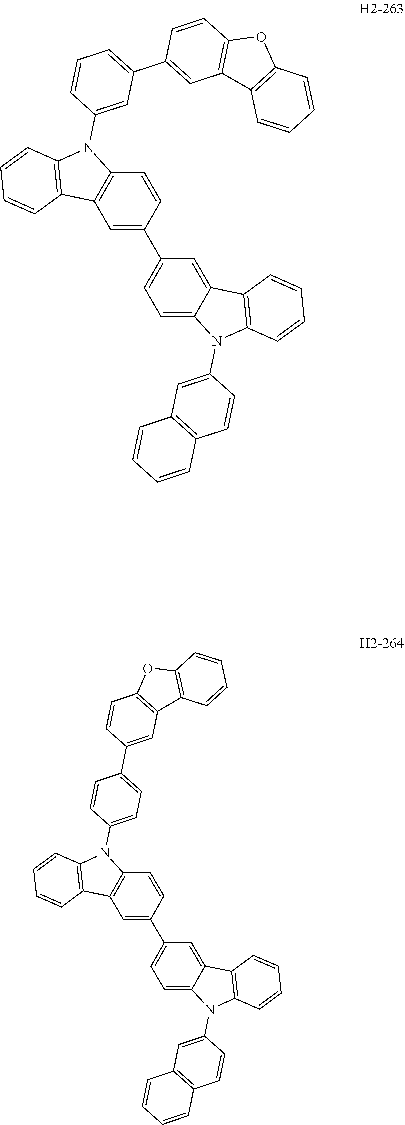 [SMILES: c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1] 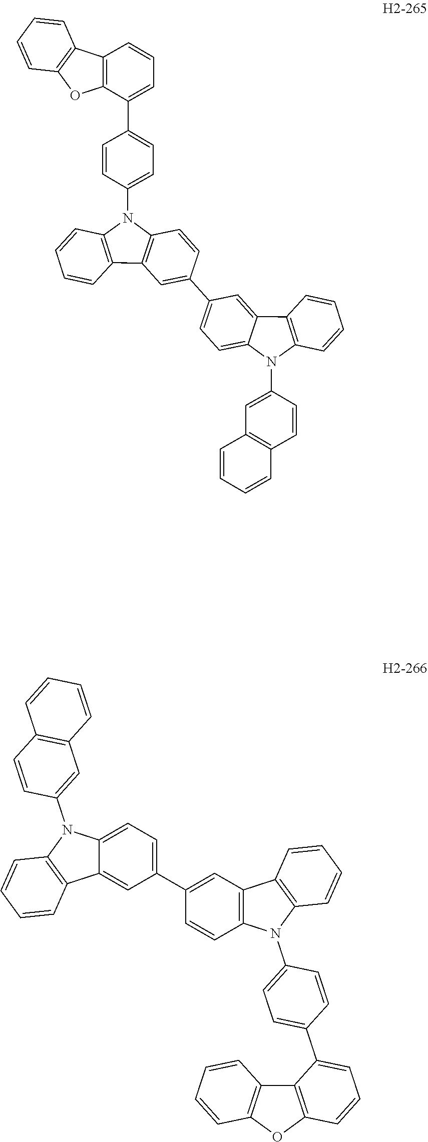 [SMILES: c1ccc2cc(-n3c4ccccc4c4cc(-c5ccc6c(c5)c5ccccc5n6-c5ccc(-c6cccc7c6oc6ccccc67)cc5)ccc43)ccc2c1 ; c1ccc2cc(-n3c4ccccc4c4cc(-c5ccc6c(c5)c5ccccc5n6-c5ccc(-c6cccc7oc8ccccc8c67)cc5)ccc43)ccc2c1] 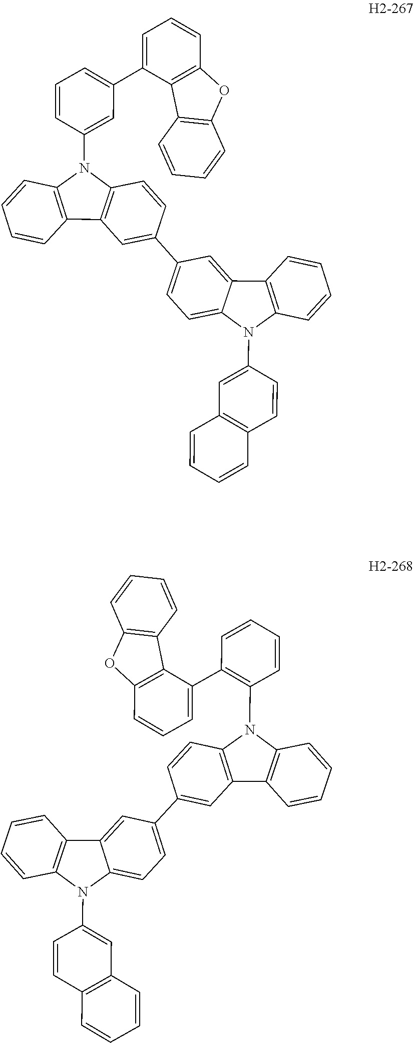 [SMILES: c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1] 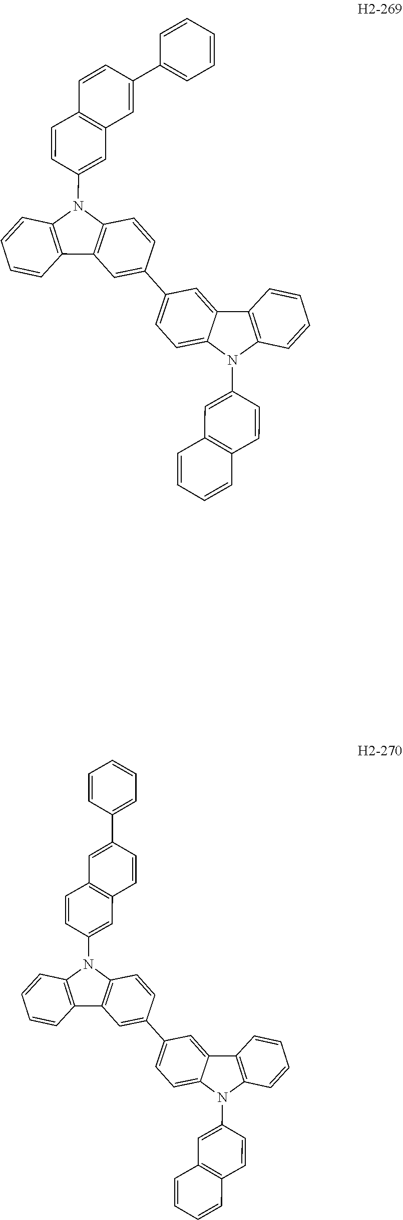 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1] 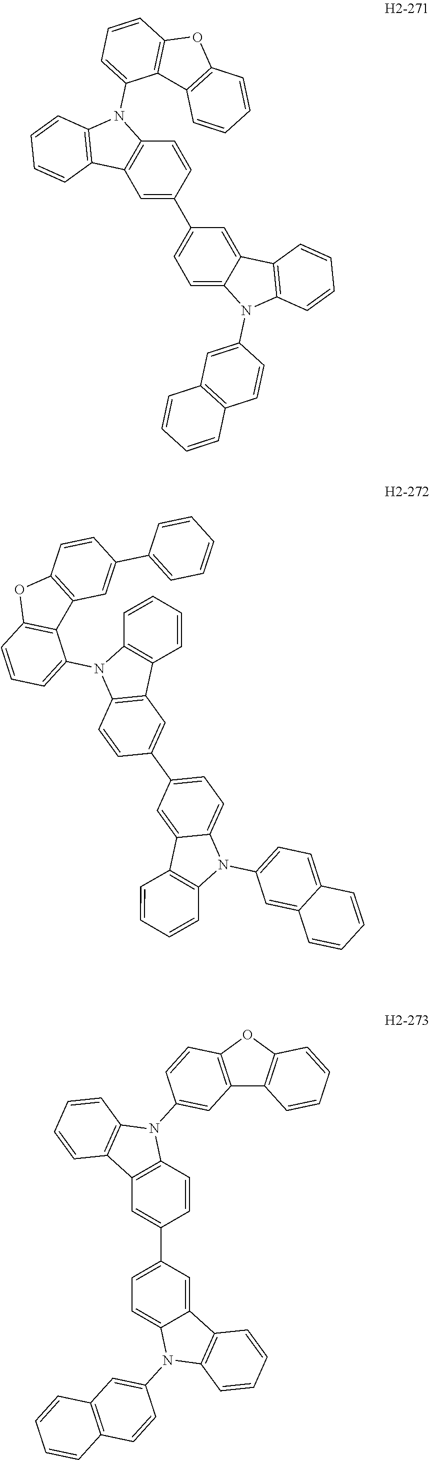 [SMILES: c1ccc2cc(-n3c4ccccc4c4cc(-c5ccc6c(c5)c5ccccc5n6-c5cccc6oc7ccccc7c56)ccc43)ccc2c1 ; c1ccc(-c2ccc3oc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6oc7ccccc7c6c5)ccc43)ccc2c1] 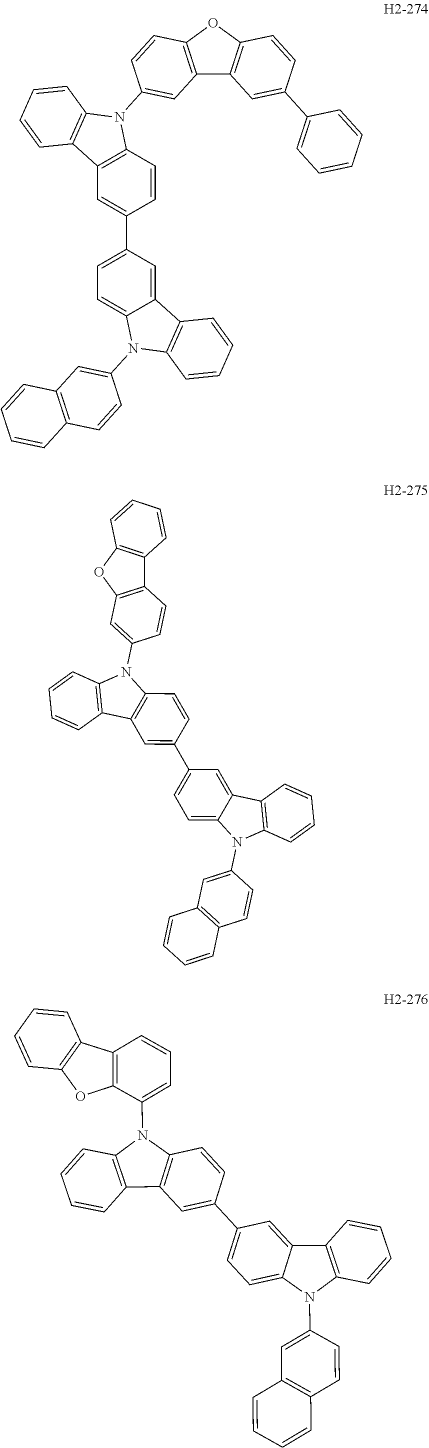 [SMILES: c1ccc(-c2ccc3oc4ccc(-n5c6ccccc6c6cc(-c7ccc8c(c7)c7ccccc7n8-c7ccc8ccccc8c7)ccc65)cc4c3c2)cc1 ; c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1] 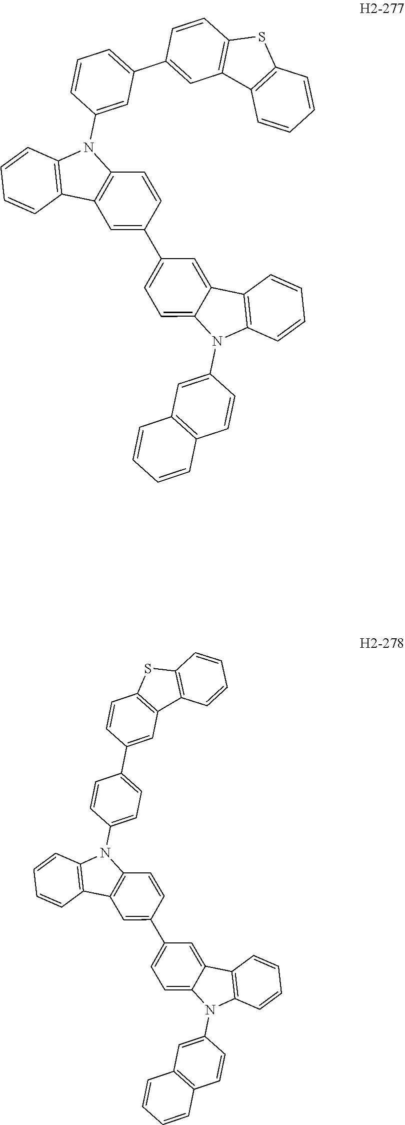 [SMILES: c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1] 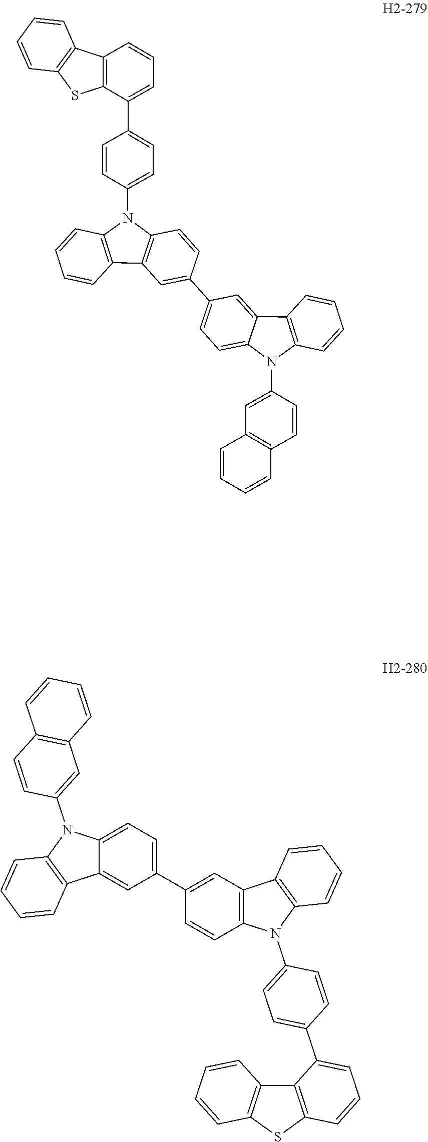 [SMILES: c1ccc2cc(-n3c4ccccc4c4cc(-c5ccc6c(c5)c5ccccc5n6-c5ccc(-c6cccc7c6sc6ccccc67)cc5)ccc43)ccc2c1 ; c1ccc2cc(-n3c4ccccc4c4cc(-c5ccc6c(c5)c5ccccc5n6-c5ccc(-c6cccc7sc8ccccc8c67)cc5)ccc43)ccc2c1] 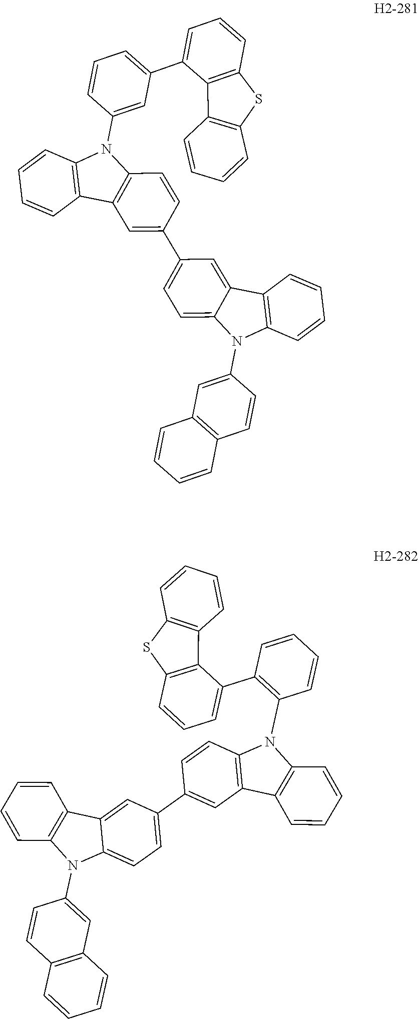 [SMILES: c1cc(-c2cccc3sc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3sc4ccccc4c23)c1] 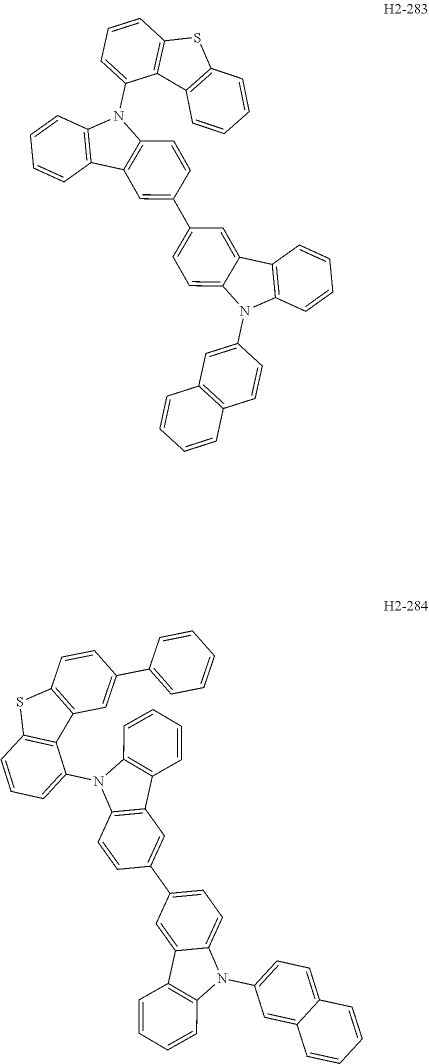 [SMILES: c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1] 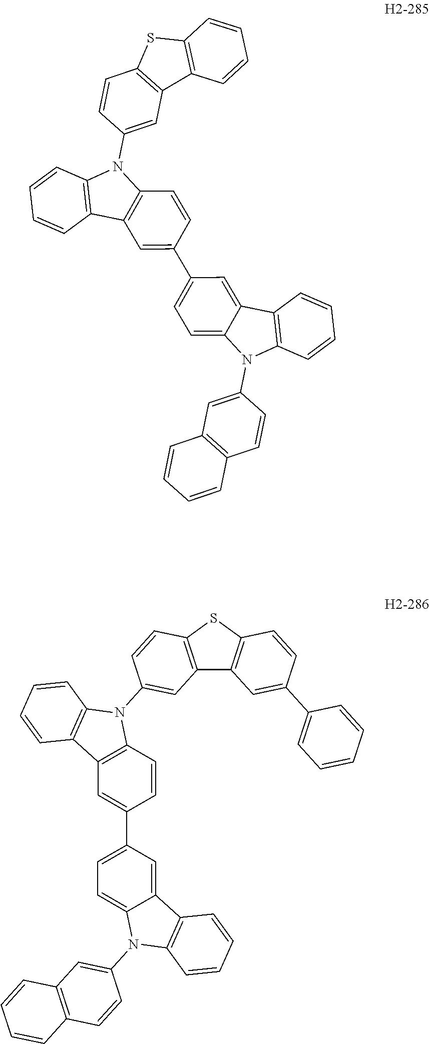 [SMILES: c1ccc2cc(-n3c4ccccc4c4cc(-c5ccc6c(c5)c5ccccc5n6-c5ccc6sc7ccccc7c6c5)ccc43)ccc2c1 ; c1ccc(-c2ccc3sc4ccc(-n5c6ccccc6c6cc(-c7ccc8c(c7)c7ccccc7n8-c7ccc8ccccc8c7)ccc65)cc4c3c2)cc1] 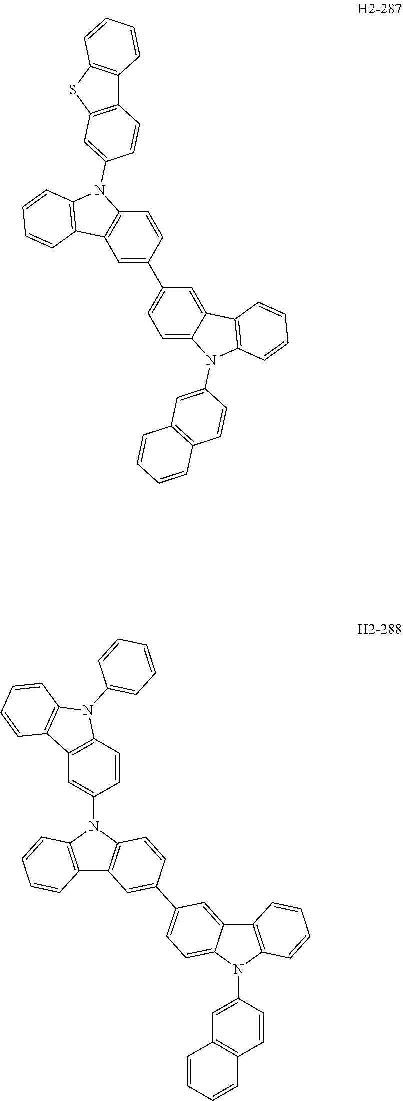 [SMILES: c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1] 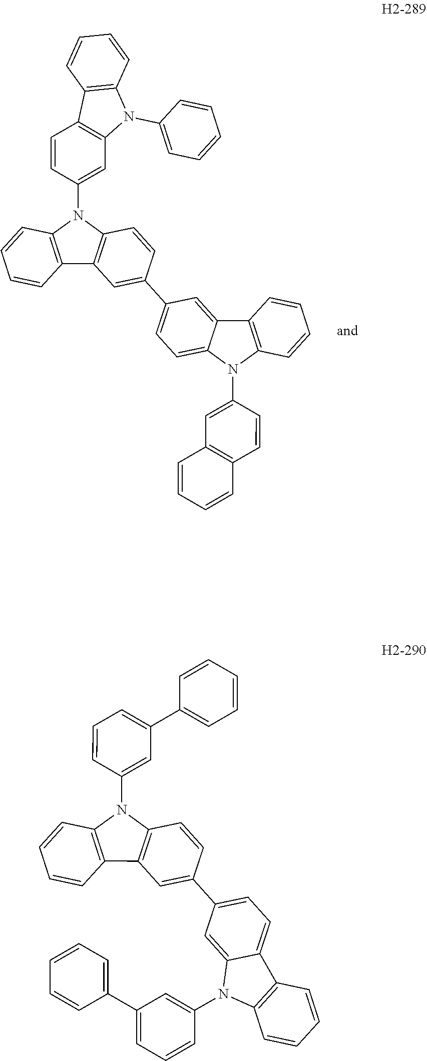 [SMILES: c1ccc(-n2c3ccccc3c3ccc(-n4c5ccccc5c5cc(-c6ccc7c(c6)c6ccccc6n7-c6ccc7ccccc7c6)ccc54)cc32)cc1 ; c1ccc(-c2cccc(-n3c4ccccc4c4cc(-c5ccc6c7ccccc7n(-c7cccc(-c8ccccc8)c7)c6c5)ccc43)c2)cc1]
In the compounds above, Dn means that n number of hydrogens is replaced with deuterium, wherein n represents an integer of 1 or more and the upper limit of n is determined by the number of hydrogens that can be substituted in each compound.

14. An organic electroluminescent device comprising: a first electrode; a second electrode; and at least one light-emitting layer(s) between the first electrode and the second electrode, wherein the at least one light-emitting layer(s) comprises the plurality of host materials according to claim 1.

15. An organic electroluminescent compound represented by the following Formula 1′: 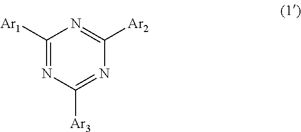
wherein,
Ar₁ and Ar₂ each independently represent, a substituted or unsubstituted (C6-C30)aryl or a substituted or unsubstituted (3- to 30-membered)heteroaryl;
Ar₃ is a carbazole group represented by the following Formula 1-1; 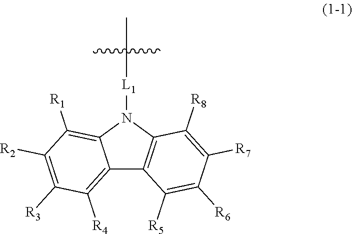
wherein,
L₁ represents a single bond or (C6-C30)arylene unsubstituted or substituted with deuterium;
R₁ to R₈ each independently represent, hydrogen, deuterium, or (C6-C30)aryl unsubstituted or substituted with deuterium, (C1-C30)alkyl, (C6-C30)aryl, or a combination thereof; provided that at least one of R₁ to R₄ is represented by the following Formula 1-11; 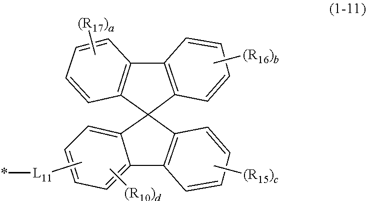
wherein,
L₁₁ represents a single bond or (C6-C30)arylene unsubstituted or substituted with deuterium;
R₁₀ and R₁₅ to R₁₇ each independently represent, hydrogen, deuterium, or (C6-C30)aryl unsubstituted or substituted with deuterium; and
a to c each independently represent, an integer of 1 to 4; and d is an integer of 1 to 3;
when a to d are 2 or more, each of R₁₀ and each of R₁₅ to each of R₁₇ may be the same or different;
provided that the following compound is excluded from the Formula 1′.

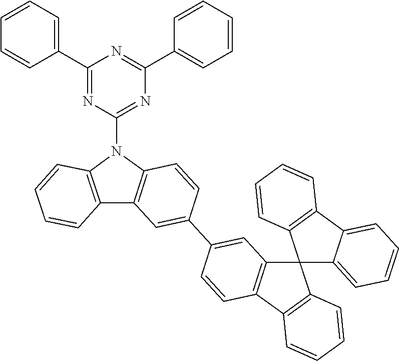 [SMILES: c1ccc(-c2nc(-c3ccccc3)nc(-n3c4ccccc4c4cc(-c5ccc6c(c5)C5(c7ccccc7-c7ccccc75)c5ccccc5-6)ccc43)n2)cc1]

16. The organic electroluminescent compound according to claim 15, wherein the organic electroluminescent compound represented by Formula 1′ satisfies the following <Condition 1> or <Condition 2>:
<Condition 1> Ar₁ and Ar₂ are asymmetric to each other;
<Condition 2> L₁ represents (C6-C30)arylene unsubstituted or substituted with deuterium.

17. The organic electroluminescent compound according to claim 15, wherein the compound represented by Formula 1′ is selected from the following compounds: 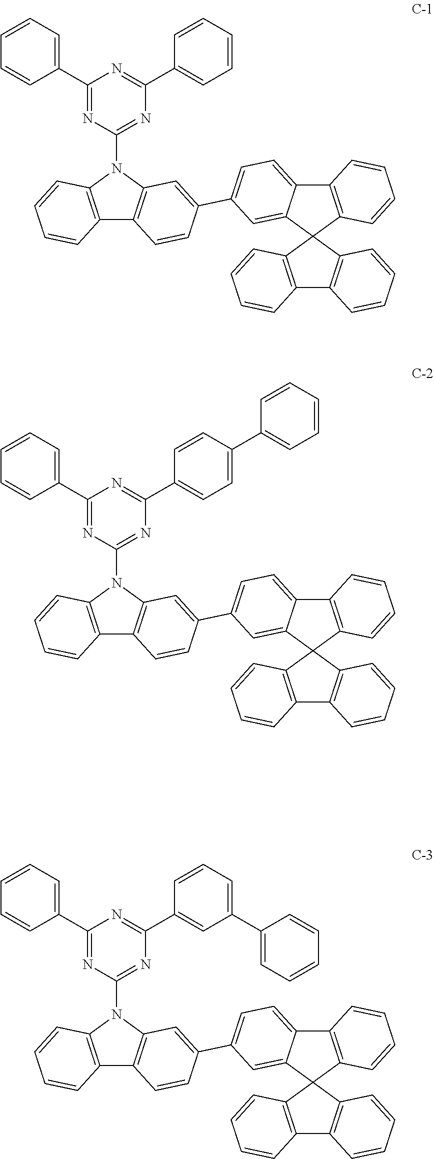 [SMILES: c1ccc(-c2nc(-c3ccccc3)nc(-n3c4ccccc4c4ccc(-c5ccc6c(c5)C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1] 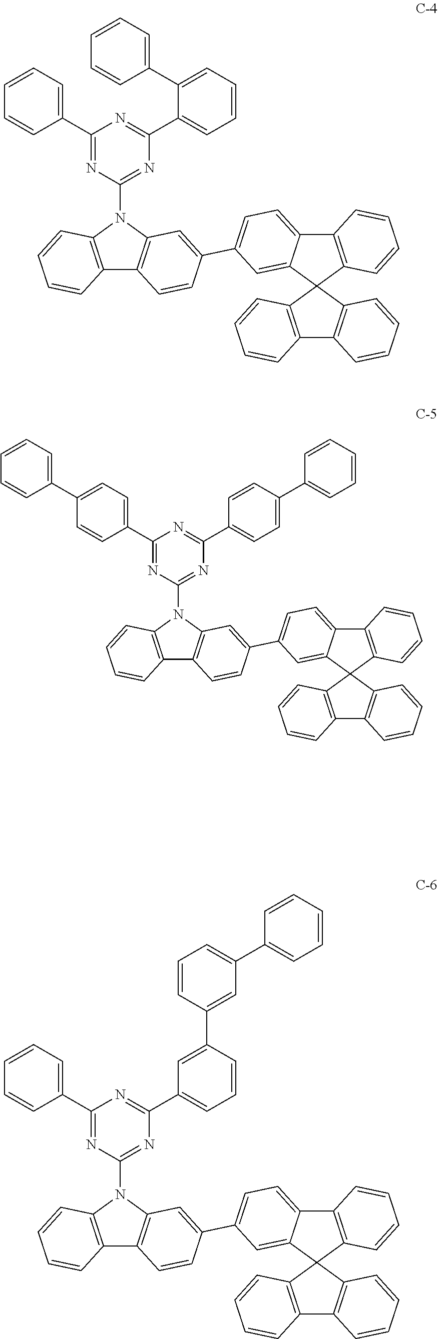 [SMILES: c1ccc(-c2nc(-c3ccccc3-c3ccccc3)nc(-n3c4ccccc4c4ccc(-c5ccc6c(c5)C5(c7ccccc7-c7ccccc75)c5ccccc5-6)cc43)n2)cc1 ; c1ccc(-c2ccc(-c3nc(-c4ccc(-c5ccccc5)cc4)nc(-n4c5ccccc5c5ccc(-c6ccc7c(c6)C6(c8ccccc8-c8ccccc86)c6ccccc6-7)cc54)n3)cc2)cc1 ; c1ccc(-c2cccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)c2)cc1] 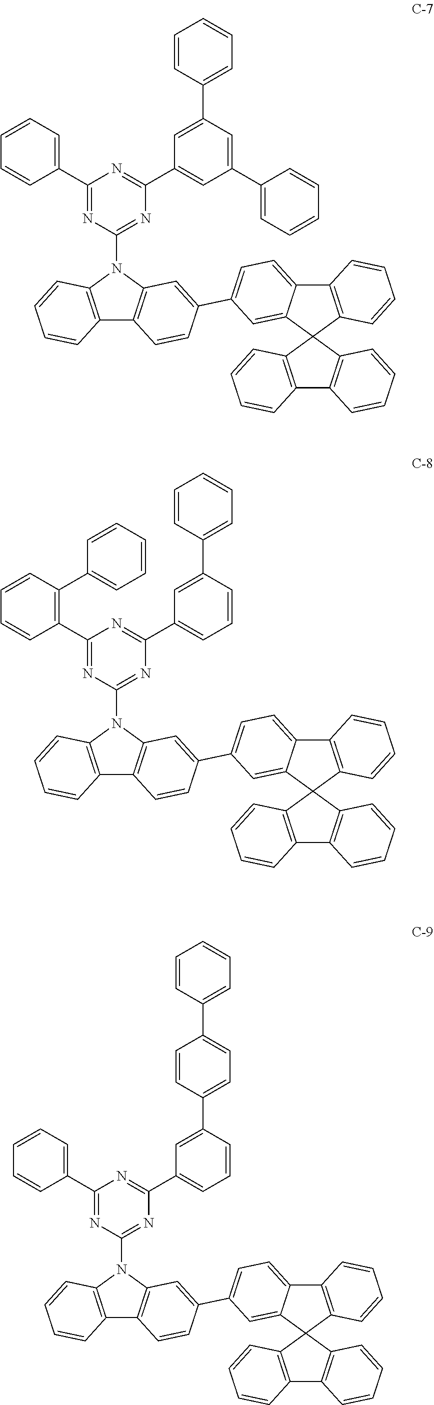 [SMILES: c1ccc(-c2cc(-c3ccccc3)cc(-c3nc(-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2cccc(-c3nc(-c4ccccc4-c4ccccc4)nc(-n4c5ccccc5c5ccc(-c6ccc7c(c6)C6(c8ccccc8-c8ccccc86)c6ccccc6-7)cc54)n3)c2)cc1 ; c1ccc(-c2ccc(-c3cccc(-c4nc(-c5ccccc5)nc(-n5c6ccccc6c6ccc(-c7ccc8c(c7)C7(c9ccccc9-c9ccccc97)c7ccccc7-8)cc65)n4)c3)cc2)cc1] 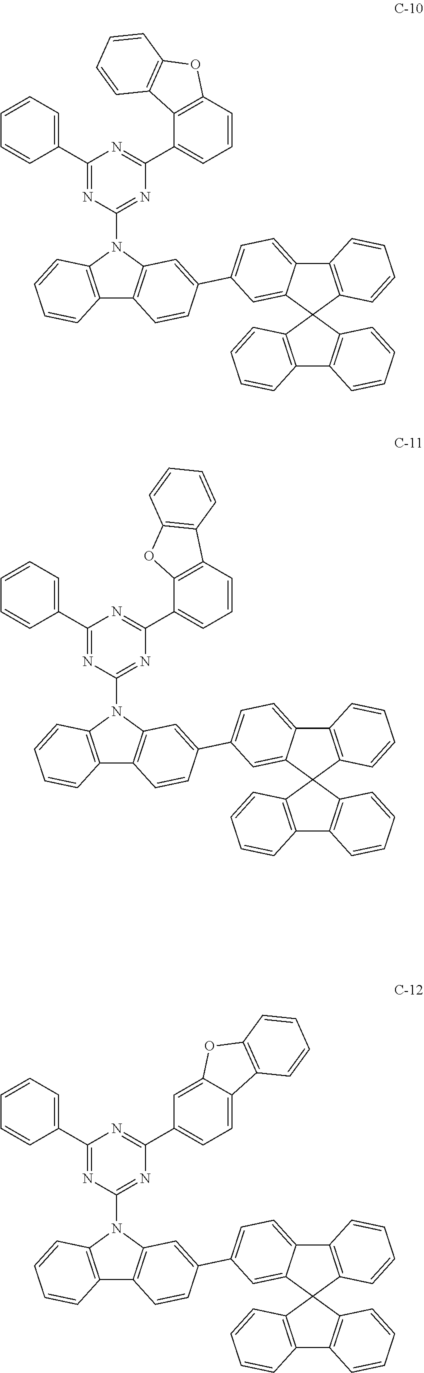 [SMILES: c1ccc(-c2nc(-c3cccc4oc5ccccc5c34)nc(-n3c4ccccc4c4ccc(-c5ccc6c(c5)C5(c7ccccc7-c7ccccc75)c5ccccc5-6)cc43)n2)cc1 ; c1ccc(-c2nc(-c3cccc4c3oc3ccccc34)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccc4c(c3)oc3ccccc34)nc(-n3c4ccccc4c4ccc(-c5ccc6c(c5)C5(c7ccccc7-c7ccccc75)c5ccccc5-6)cc43)n2)cc1] 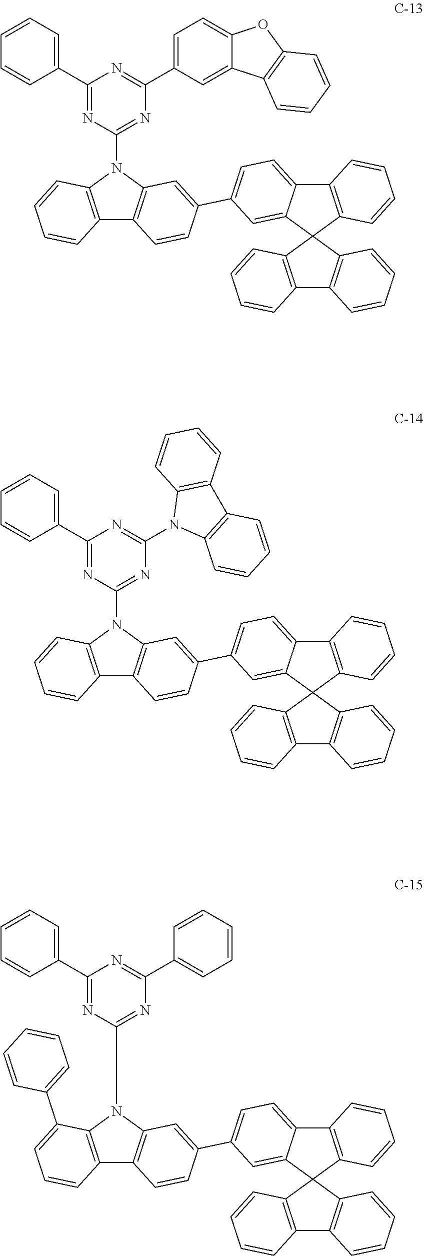 [SMILES: c1ccc(-c2nc(-c3ccc4oc5ccccc5c4c3)nc(-n3c4ccccc4c4ccc(-c5ccc6c(c5)C5(c7ccccc7-c7ccccc75)c5ccccc5-6)cc43)n2)cc1 ; c1ccc(-c2nc(-n3c4ccccc4c4ccccc43)nc(-n3c4ccccc4c4ccc(-c5ccc6c(c5)C5(c7ccccc7-c7ccccc75)c5ccccc5-6)cc43)n2)cc1 ; c1ccc(-c2nc(-c3ccccc3)nc(-n3c4cc(-c5ccc6c(c5)C5(c7ccccc7-c7ccccc75)c5ccccc5-6)ccc4c4cccc(-c5ccccc5)c43)n2)cc1] 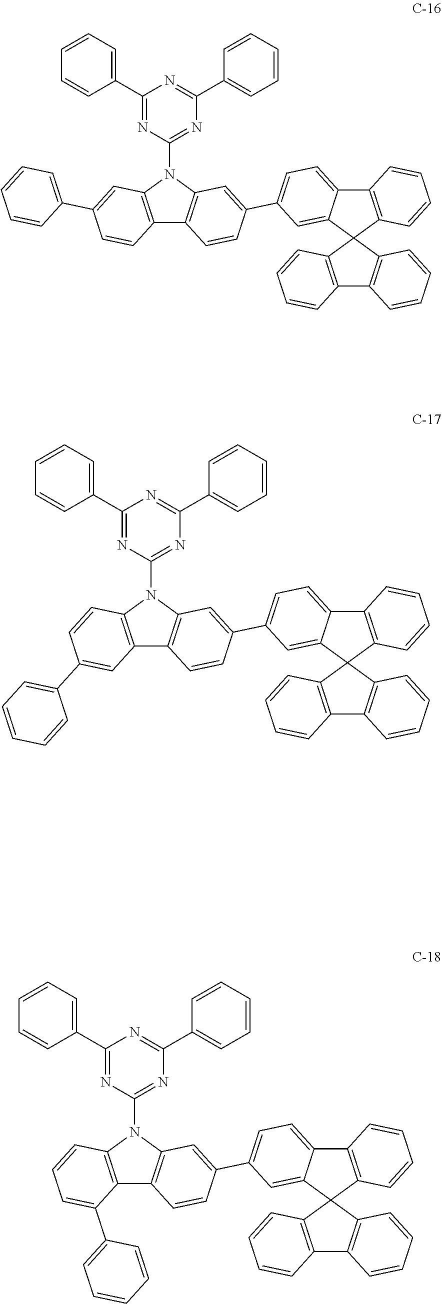 [SMILES: c1ccc(-c2ccc3c4ccc(-c5ccc6c(c5)C5(c7ccccc7-c7ccccc75)c5ccccc5-6)cc4n(-c4nc(-c5ccccc5)nc(-c5ccccc5)n4)c3c2)cc1 ; c1ccc(-c2ccc3c(c2)c2ccc(-c4ccc5c(c4)C4(c6ccccc6-c6ccccc64)c4ccccc4-5)cc2n3-c2nc(-c3ccccc3)nc(-c3ccccc3)n2)cc1 ; c1ccc(-c2nc(-c3ccccc3)nc(-n3c4cc(-c5ccc6c(c5)C5(c7ccccc7-c7ccccc75)c5ccccc5-6)ccc4c4c(-c5ccccc5)cccc43)n2)cc1] 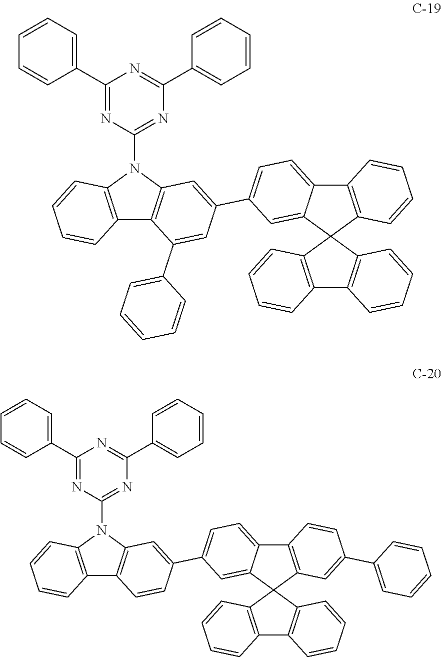 [SMILES: c1ccc(-c2nc(-c3ccccc3)nc(-n3c4ccccc4c4c(-c5ccccc5)cc(-c5ccc6c(c5)C5(c7ccccc7-c7ccccc75)c5ccccc5-6)cc43)n2)cc1 ; c1ccc(-c2ccc3c(c2)C2(c4ccccc4-c4ccccc42)c2cc(-c4ccc5c6ccccc6n(-c6nc(-c7ccccc7)nc(-c7ccccc7)n6)c5c4)ccc2-3)cc1] 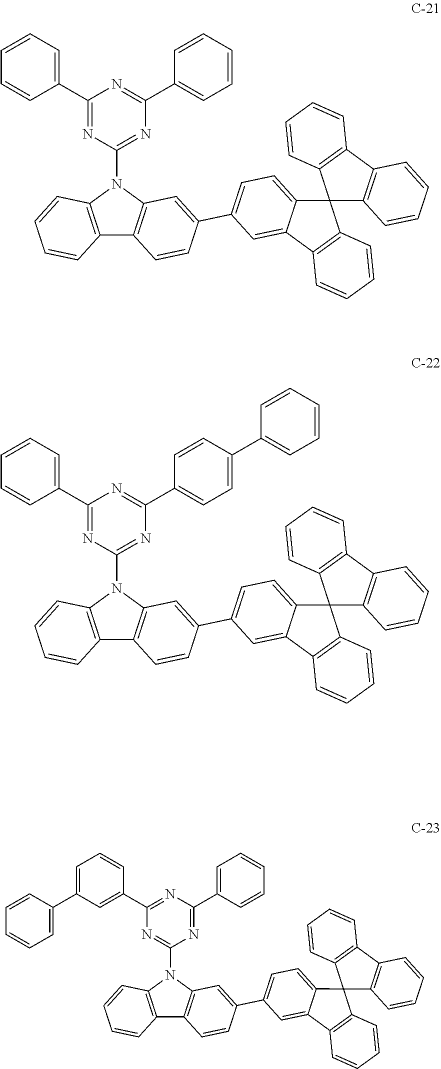 [SMILES: c1ccc(-c2nc(-c3ccccc3)nc(-n3c4ccccc4c4ccc(-c5ccc6c(c5)-c5ccccc5C65c6ccccc6-c6ccccc65)cc43)n2)cc1 ; c1ccc(-c2ccc(-c3nc(-c4ccccc4)nc(-n4c5ccccc5c5ccc(-c6ccc7c(c6)-c6ccccc6C76c7ccccc7-c7ccccc76)cc54)n3)cc2)cc1 ; c1ccc(-c2cccc(-c3nc(-c4ccccc4)nc(-n4c5ccccc5c5ccc(-c6ccc7c(c6)-c6ccccc6C76c7ccccc7-c7ccccc76)cc54)n3)c2)cc1] 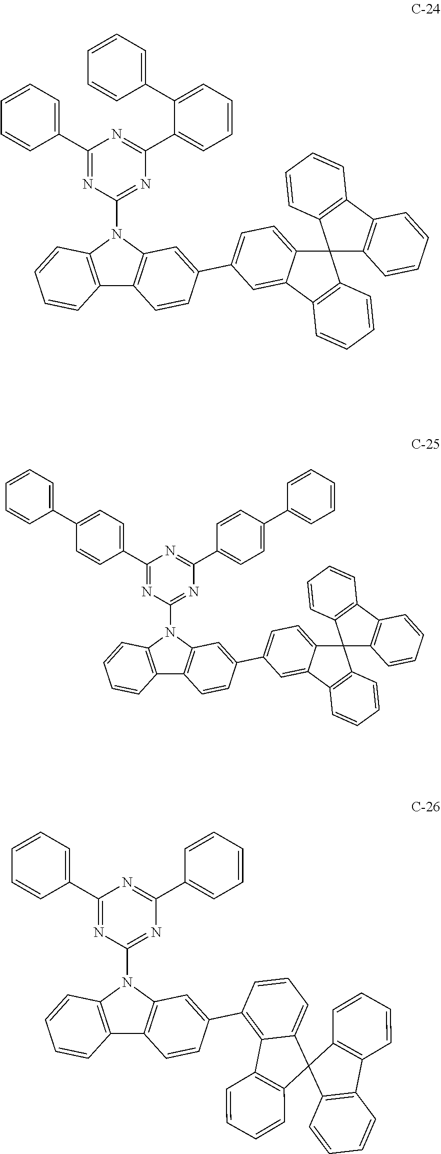 [SMILES: c1ccc(-c2nc(-c3ccccc3-c3ccccc3)nc(-n3c4ccccc4c4ccc(-c5ccc6c(c5)-c5ccccc5C65c6ccccc6-c6ccccc65)cc43)n2)cc1 ; c1ccc(-c2ccc(-c3nc(-c4ccc(-c5ccccc5)cc4)nc(-n4c5ccccc5c5ccc(-c6ccc7c(c6)-c6ccccc6C76c7ccccc7-c7ccccc76)cc54)n3)cc2)cc1 ; c1ccc(-c2nc(-c3ccccc3)nc(-n3c4ccccc4c4ccc(-c5cccc6c5-c5ccccc5C65c6ccccc6-c6ccccc65)cc43)n2)cc1] 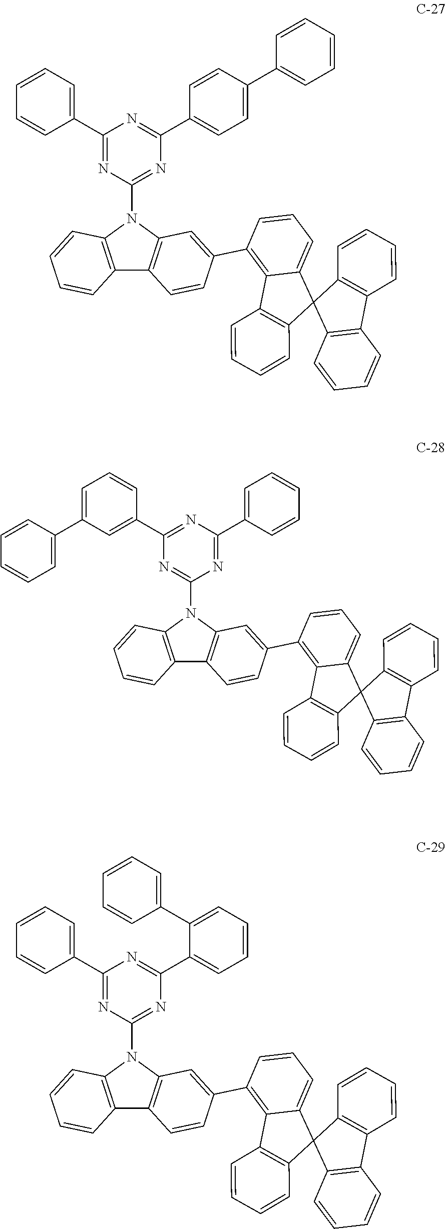 [SMILES: c1ccc(-c2ccc(-c3nc(-c4ccccc4)nc(-n4c5ccccc5c5ccc(-c6cccc7c6-c6ccccc6C76c7ccccc7-c7ccccc76)cc54)n3)cc2)cc1 ; c1ccc(-c2cccc(-c3nc(-c4ccccc4)nc(-n4c5ccccc5c5ccc(-c6cccc7c6-c6ccccc6C76c7ccccc7-c7ccccc76)cc54)n3)c2)cc1 ; c1ccc(-c2nc(-c3ccccc3-c3ccccc3)nc(-n3c4ccccc4c4ccc(-c5cccc6c5-c5ccccc5C65c6ccccc6-c6ccccc65)cc43)n2)cc1] 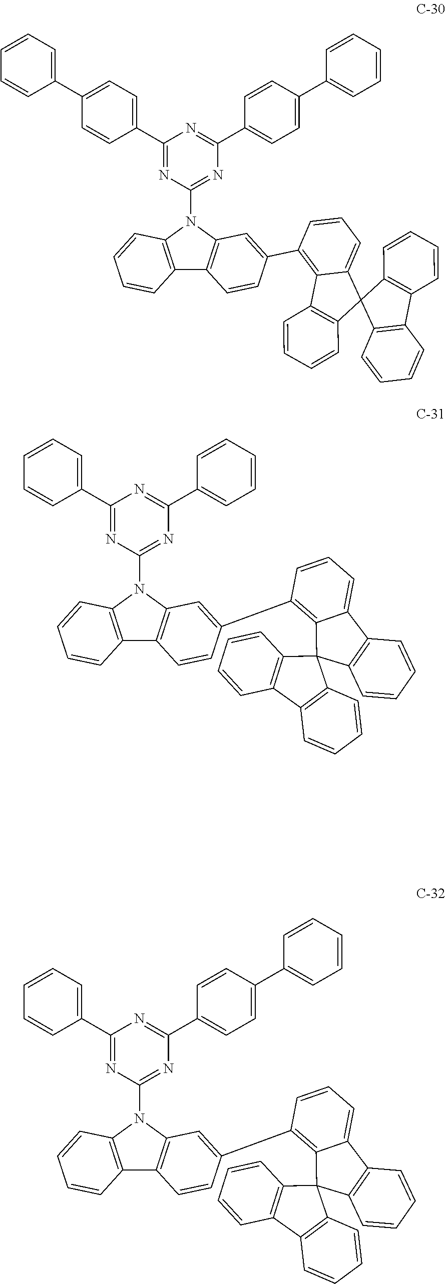 [SMILES: c1ccc(-c2ccc(-c3nc(-c4ccc(-c5ccccc5)cc4)nc(-n4c5ccccc5c5ccc(-c6cccc7c6-c6ccccc6C76c7ccccc7-c7ccccc76)cc54)n3)cc2)cc1 ; c1ccc(-c2nc(-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccccc4)nc(-n4c5ccccc5c5ccc(-c6cccc7c6C6(c8ccccc8-c8ccccc86)c6ccccc6-7)cc54)n3)cc2)cc1] 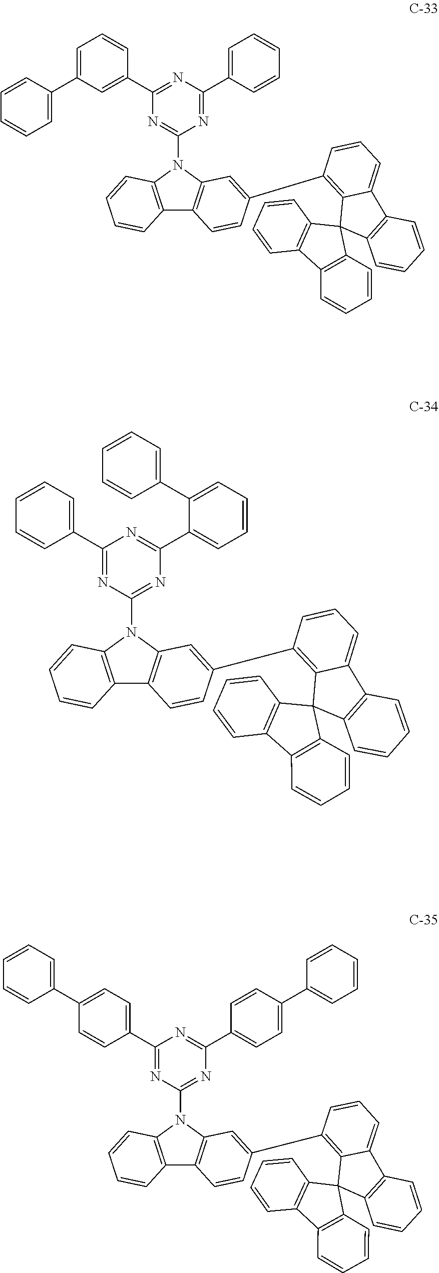 [SMILES: c1ccc(-c2cccc(-c3nc(-c4ccccc4)nc(-n4c5ccccc5c5ccc(-c6cccc7c6C6(c8ccccc8-c8ccccc86)c6ccccc6-7)cc54)n3)c2)cc1 ; c1ccc(-c2nc(-c3ccccc3-c3ccccc3)nc(-n3c4ccccc4c4ccc(-c5cccc6c5C5(c7ccccc7-c7ccccc75)c5ccccc5-6)cc43)n2)cc1 ; c1ccc(-c2ccc(-c3nc(-c4ccc(-c5ccccc5)cc4)nc(-n4c5ccccc5c5ccc(-c6cccc7c6C6(c8ccccc8-c8ccccc86)c6ccccc6-7)cc54)n3)cc2)cc1] 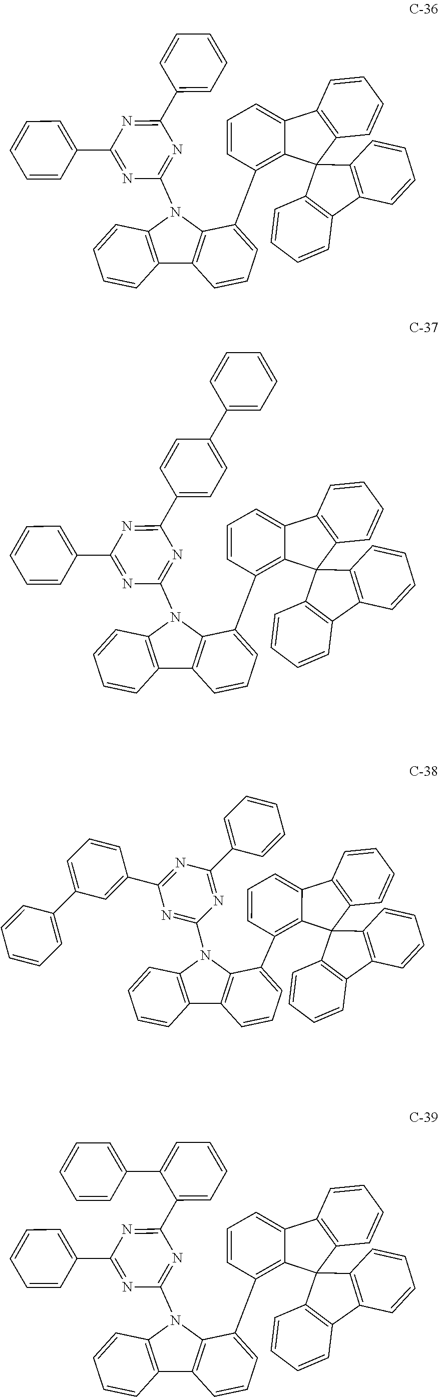 [SMILES: c1ccc(-c2nc(-c3ccccc3)nc(-n3c4ccccc4c4cccc(-c5cccc6c5C5(c7ccccc7-c7ccccc75)c5ccccc5-6)c43)n2)cc1 ; c1ccc(-c2ccc(-c3nc(-c4ccccc4)nc(-n4c5ccccc5c5cccc(-c6cccc7c6C6(c8ccccc8-c8ccccc86)c6ccccc6-7)c54)n3)cc2)cc1 ; c1ccc(-c2cccc(-c3nc(-c4ccccc4)nc(-n4c5ccccc5c5cccc(-c6cccc7c6C6(c8ccccc8-c8ccccc86)c6ccccc6-7)c54)n3)c2)cc1 ; c1ccc(-c2nc(-c3ccccc3-c3ccccc3)nc(-n3c4ccccc4c4cccc(-c5cccc6c5C5(c7ccccc7-c7ccccc75)c5ccccc5-6)c43)n2)cc1] 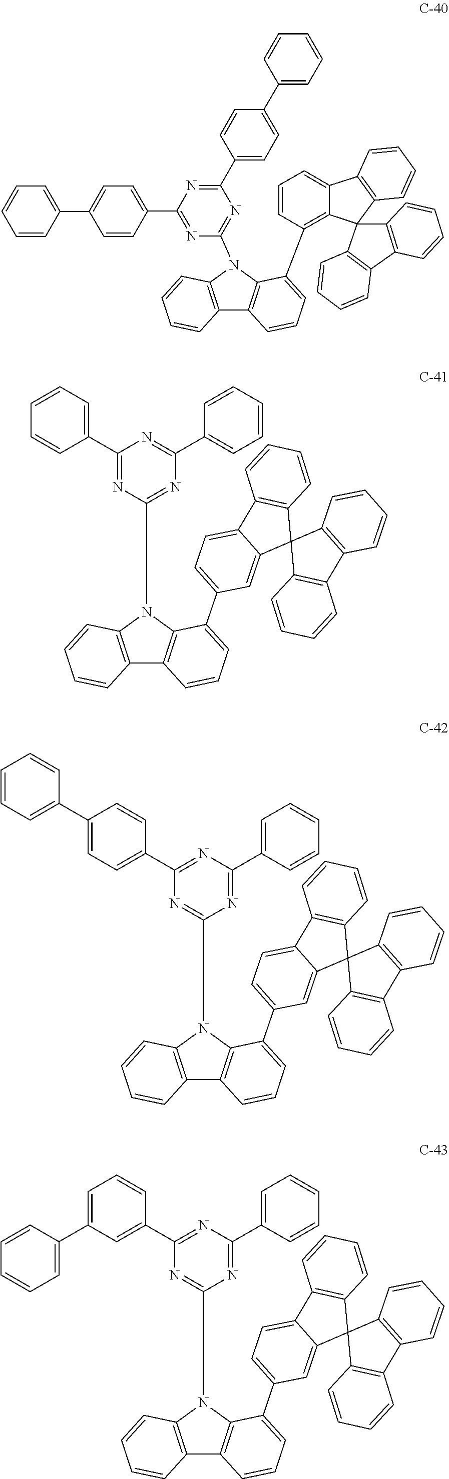 [SMILES: c1ccc(-c2ccc(-c3nc(-c4ccc(-c5ccccc5)cc4)nc(-n4c5ccccc5c5cccc(-c6cccc7c6C6(c8ccccc8-c8ccccc86)c6ccccc6-7)c54)n3)cc2)cc1 ; c1ccc(-c2nc(-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccccc4)nc(-n4c5ccccc5c5cccc(-c6ccc7c(c6)C6(c8ccccc8-c8ccccc86)c6ccccc6-7)c54)n3)cc2)cc1 ; c1ccc(-c2cccc(-c3nc(-c4ccccc4)nc(-n4c5ccccc5c5cccc(-c6ccc7c(c6)C6(c8ccccc8-c8ccccc86)c6ccccc6-7)c54)n3)c2)cc1] 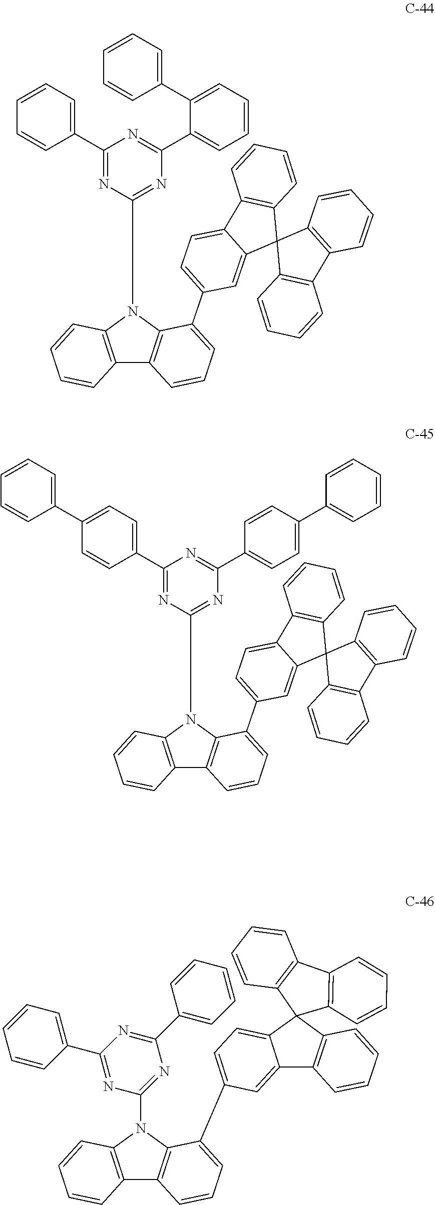 [SMILES: c1ccc(-c2nc(-c3ccccc3-c3ccccc3)nc(-n3c4ccccc4c4cccc(-c5ccc6c(c5)C5(c7ccccc7-c7ccccc75)c5ccccc5-6)c43)n2)cc1 ; c1ccc(-c2ccc(-c3nc(-c4ccc(-c5ccccc5)cc4)nc(-n4c5ccccc5c5cccc(-c6ccc7c(c6)C6(c8ccccc8-c8ccccc86)c6ccccc6-7)c54)n3)cc2)cc1 ; c1ccc(-c2nc(-c3ccccc3)nc(-n3c4ccccc4c4cccc(-c5ccc6c(c5)-c5ccccc5C65c6ccccc6-c6ccccc65)c43)n2)cc1] 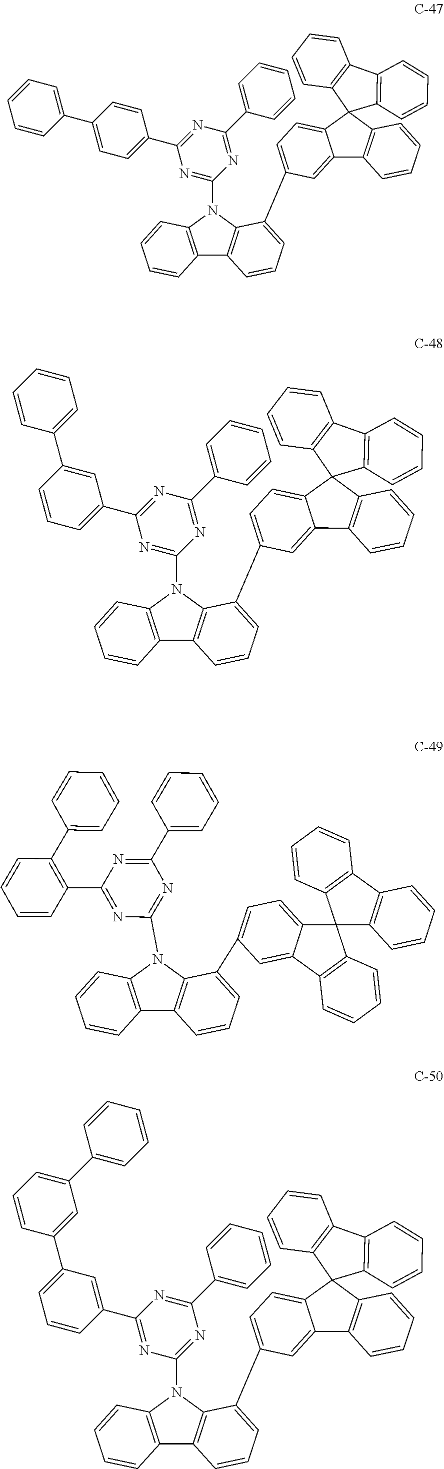 [SMILES: c1ccc(-c2ccc(-c3nc(-c4ccccc4)nc(-n4c5ccccc5c5cccc(-c6ccc7c(c6)-c6ccccc6C76c7ccccc7-c7ccccc76)c54)n3)cc2)cc1 ; c1ccc(-c2cccc(-c3nc(-c4ccccc4)nc(-n4c5ccccc5c5cccc(-c6ccc7c(c6)-c6ccccc6C76c7ccccc7-c7ccccc76)c54)n3)c2)cc1 ; c1ccc(-c2nc(-c3ccccc3-c3ccccc3)nc(-n3c4ccccc4c4cccc(-c5ccc6c(c5)-c5ccccc5C65c6ccccc6-c6ccccc65)c43)n2)cc1 ; c1ccc(-c2cccc(-c3cccc(-c4nc(-c5ccccc5)nc(-n5c6ccccc6c6cccc(-c7ccc8c(c7)-c7ccccc7C87c8ccccc8-c8ccccc87)c65)n4)c3)c2)cc1] 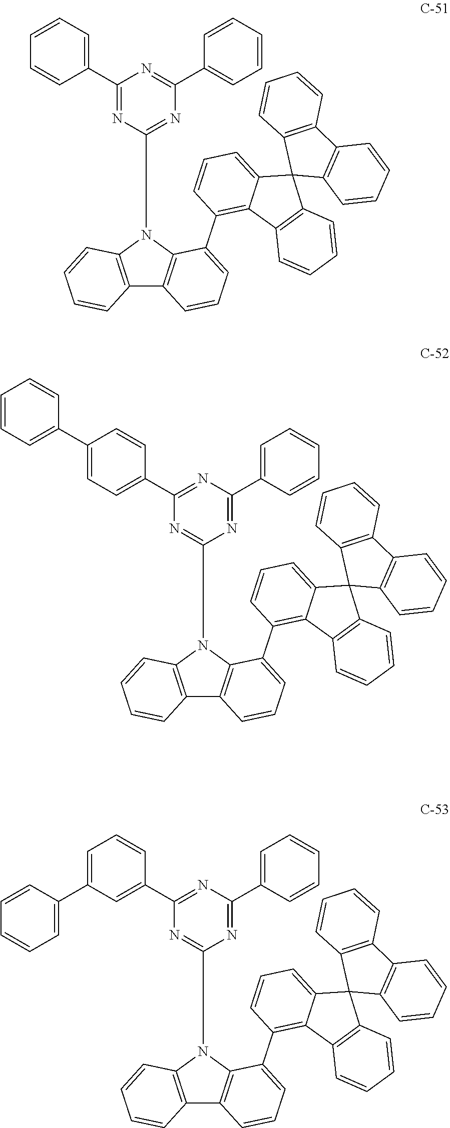 [SMILES: c1ccc(-c2nc(-c3ccccc3)nc(-n3c4ccccc4c4cccc(-c5cccc6c5-c5ccccc5C65c6ccccc6-c6ccccc65)c43)n2)cc1 ; c1ccc(-c2ccc(-c3nc(-c4ccccc4)nc(-n4c5ccccc5c5cccc(-c6cccc7c6-c6ccccc6C76c7ccccc7-c7ccccc76)c54)n3)cc2)cc1 ; c1ccc(-c2cccc(-c3nc(-c4ccccc4)nc(-n4c5ccccc5c5cccc(-c6cccc7c6-c6ccccc6C76c7ccccc7-c7ccccc76)c54)n3)c2)cc1] 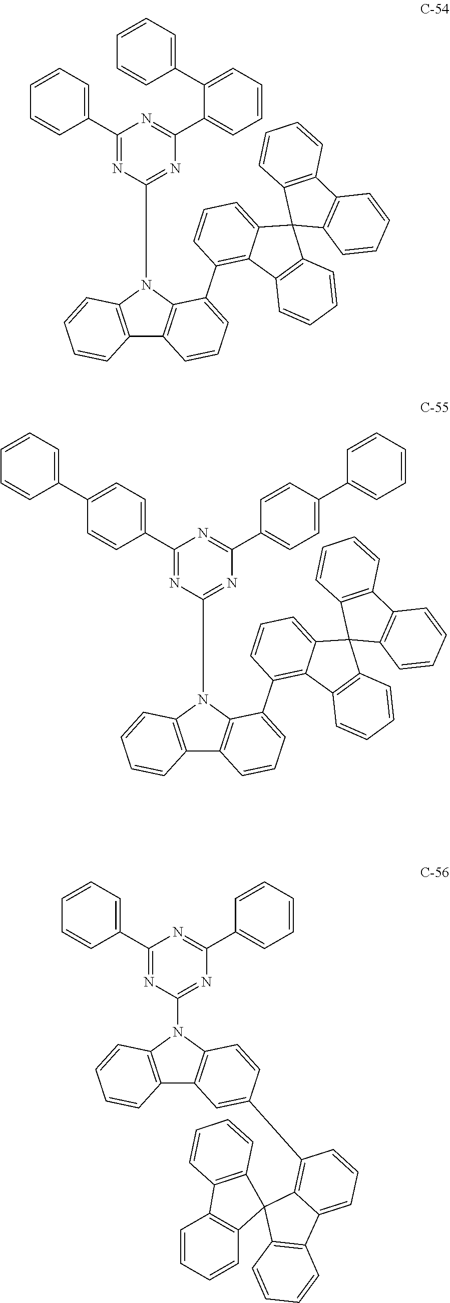 [SMILES: c1ccc(-c2nc(-c3ccccc3-c3ccccc3)nc(-n3c4ccccc4c4cccc(-c5cccc6c5-c5ccccc5C65c6ccccc6-c6ccccc65)c43)n2)cc1 ; c1ccc(-c2ccc(-c3nc(-c4ccc(-c5ccccc5)cc4)nc(-n4c5ccccc5c5cccc(-c6cccc7c6-c6ccccc6C76c7ccccc7-c7ccccc76)c54)n3)cc2)cc1 ; c1ccc(-c2nc(-c3ccccc3)nc(-n3c4ccccc4c4cc(-c5cccc6c5C5(c7ccccc7-c7ccccc75)c5ccccc5-6)ccc43)n2)cc1] 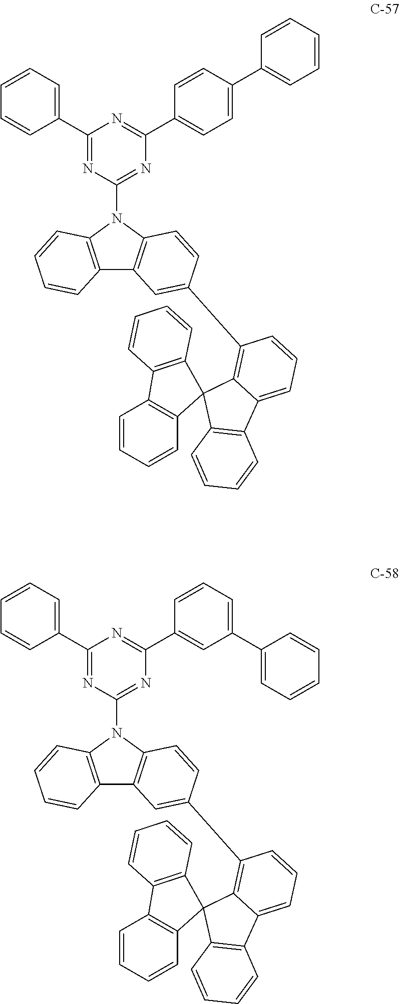 [SMILES: c1ccc(-c2ccc(-c3nc(-c4ccccc4)nc(-n4c5ccccc5c5cc(-c6cccc7c6C6(c8ccccc8-c8ccccc86)c6ccccc6-7)ccc54)n3)cc2)cc1 ; c1ccc(-c2cccc(-c3nc(-c4ccccc4)nc(-n4c5ccccc5c5cc(-c6cccc7c6C6(c8ccccc8-c8ccccc86)c6ccccc6-7)ccc54)n3)c2)cc1] 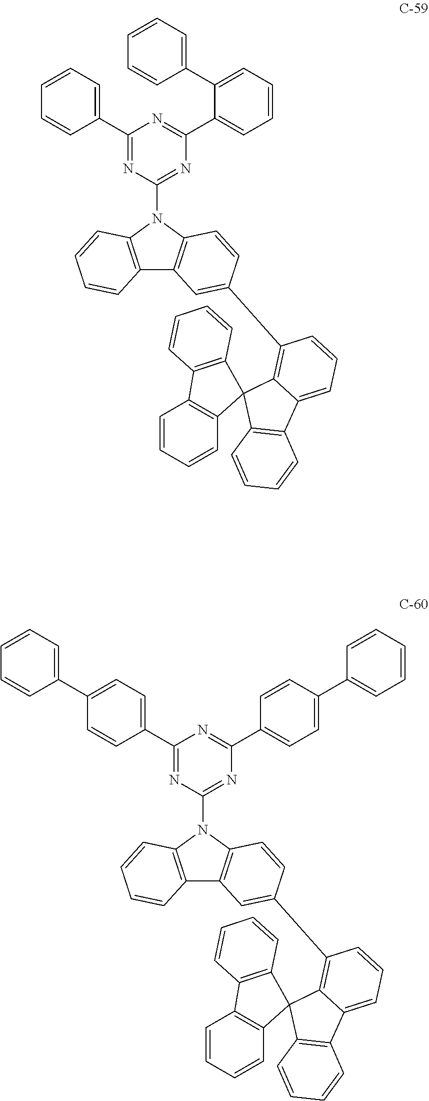 [SMILES: c1ccc(-c2nc(-c3ccccc3-c3ccccc3)nc(-n3c4ccccc4c4cc(-c5cccc6c5C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6cccc7c6C6(c8ccccc8-c8ccccc86)c6ccccc6-7)ccc54)n3)cc2)cc1] 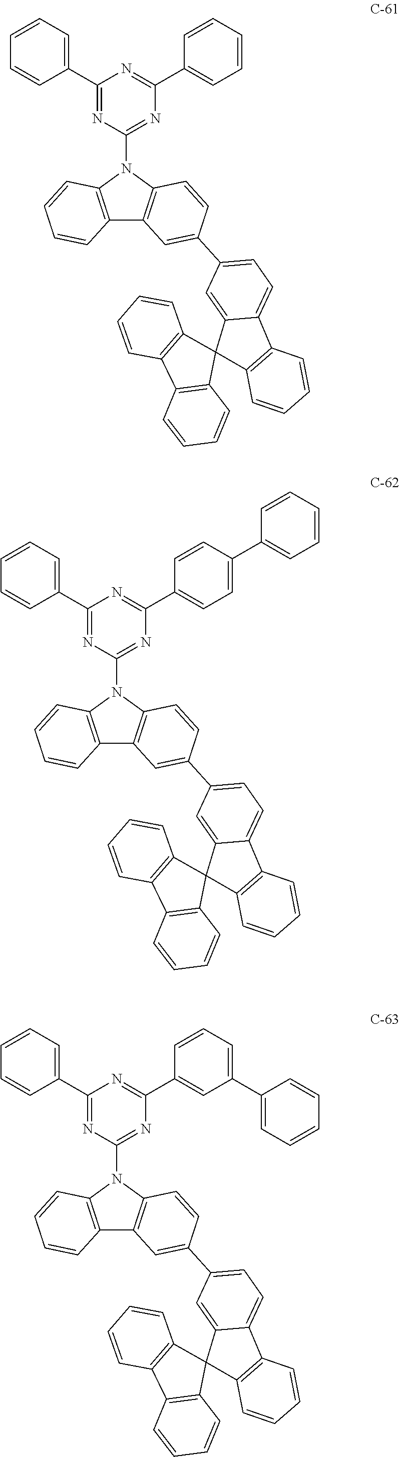 [SMILES: c1ccc(-c2nc(-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccccc4)nc(-n4c5ccccc5c5cc(-c6ccc7c(c6)C6(c8ccccc8-c8ccccc86)c6ccccc6-7)ccc54)n3)cc2)cc1 ; c1ccc(-c2cccc(-c3nc(-c4ccccc4)nc(-n4c5ccccc5c5cc(-c6ccc7c(c6)C6(c8ccccc8-c8ccccc86)c6ccccc6-7)ccc54)n3)c2)cc1] 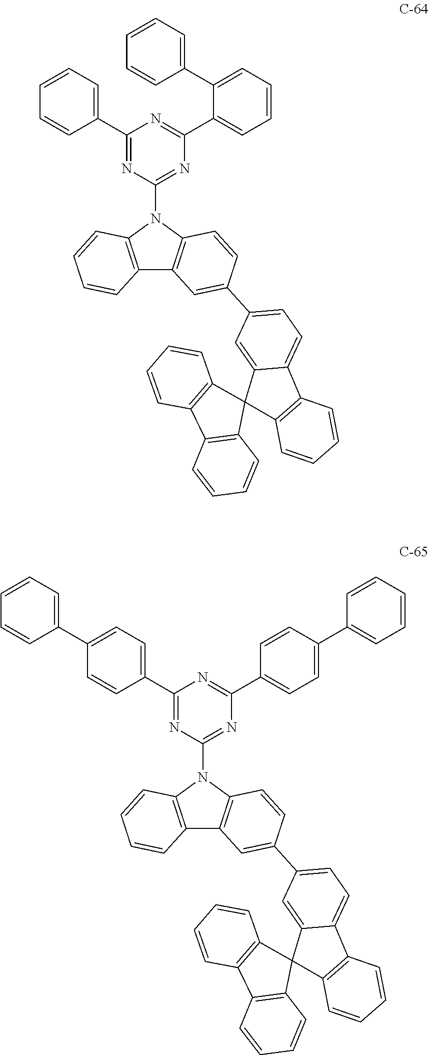 [SMILES: c1ccc(-c2nc(-c3ccccc3-c3ccccc3)nc(-n3c4ccccc4c4cc(-c5ccc6c(c5)C5(c7ccccc7-c7ccccc75)c5ccccc5-6)ccc43)n2)cc1 ; c1ccc(-c2ccc(-c3nc(-c4ccc(-c5ccccc5)cc4)nc(-n4c5ccccc5c5cc(-c6ccc7c(c6)C6(c8ccccc8-c8ccccc86)c6ccccc6-7)ccc54)n3)cc2)cc1] 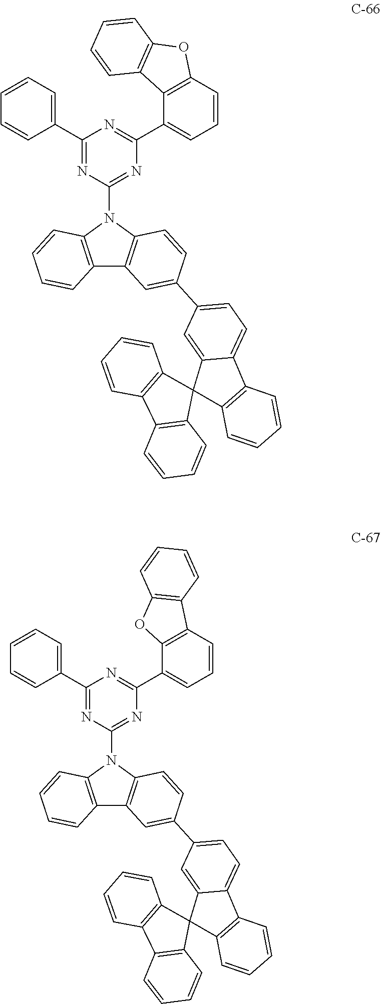 [SMILES: c1ccc(-c2nc(-c3cccc4oc5ccccc5c34)nc(-n3c4ccccc4c4cc(-c5ccc6c(c5)C5(c7ccccc7-c7ccccc75)c5ccccc5-6)ccc43)n2)cc1 ; c1ccc(-c2nc(-c3cccc4c3oc3ccccc34)nc(-n3c4ccccc4c4cc(-c5ccc6c(c5)C5(c7ccccc7-c7ccccc75)c5ccccc5-6)ccc43)n2)cc1] 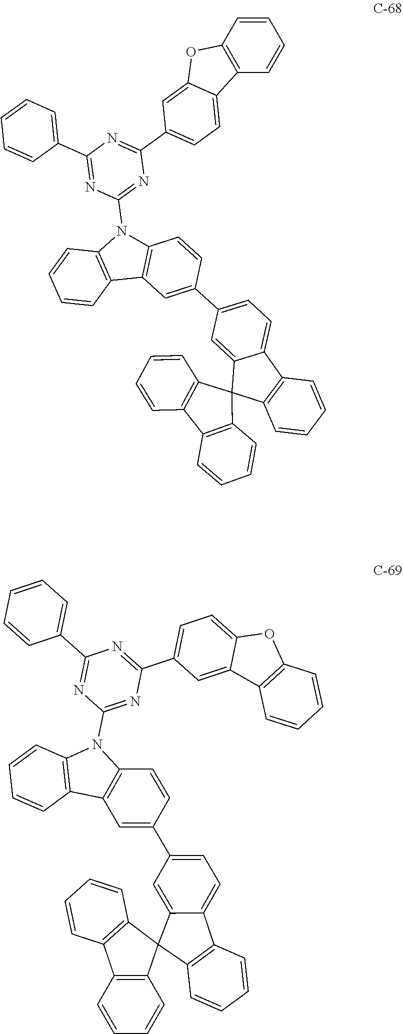 [SMILES: c1ccc(-c2nc(-c3ccc4c(c3)oc3ccccc34)nc(-n3c4ccccc4c4cc(-c5ccc6c(c5)C5(c7ccccc7-c7ccccc75)c5ccccc5-6)ccc43)n2)cc1 ; c1ccc(-c2nc(-c3ccc4oc5ccccc5c4c3)nc(-n3c4ccccc4c4cc(-c5ccc6c(c5)C5(c7ccccc7-c7ccccc75)c5ccccc5-6)ccc43)n2)cc1] 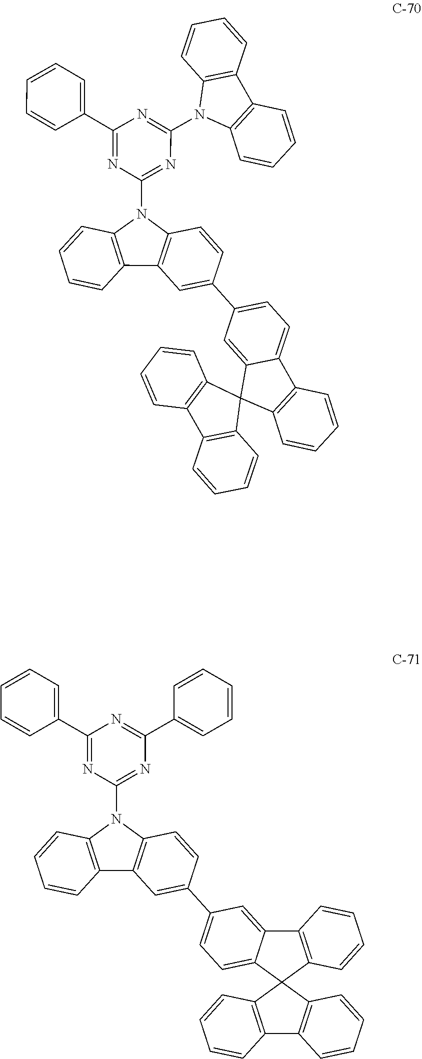 [SMILES: c1ccc(-c2nc(-n3c4ccccc4c4ccccc43)nc(-n3c4ccccc4c4cc(-c5ccc6c(c5)C5(c7ccccc7-c7ccccc75)c5ccccc5-6)ccc43)n2)cc1 ; c1ccc(-c2nc(-c3ccccc3)nc(-n3c4ccccc4c4cc(-c5ccc6c(c5)-c5ccccc5C65c6ccccc6-c6ccccc65)ccc43)n2)cc1] 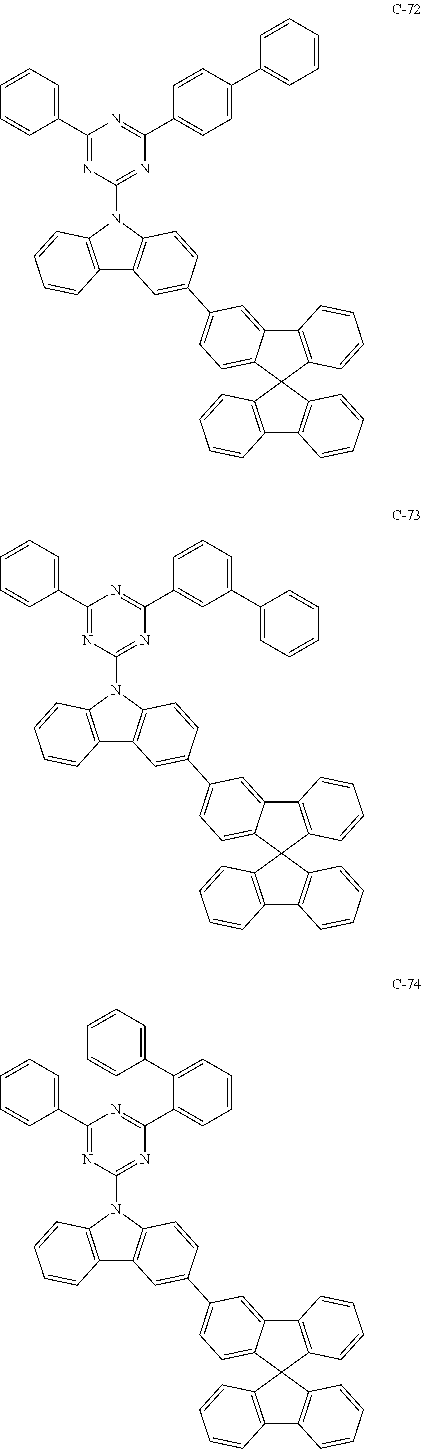 [SMILES: c1ccc(-c2ccc(-c3nc(-c4ccccc4)nc(-n4c5ccccc5c5cc(-c6ccc7c(c6)-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6ccc7c(c6)-c6ccccc6C76c7ccccc7-c7ccccc76)ccc54)n3)c2)cc1 ; c1ccc(-c2nc(-c3ccccc3-c3ccccc3)nc(-n3c4ccccc4c4cc(-c5ccc6c(c5)-c5ccccc5C65c6ccccc6-c6ccccc65)ccc43)n2)cc1] 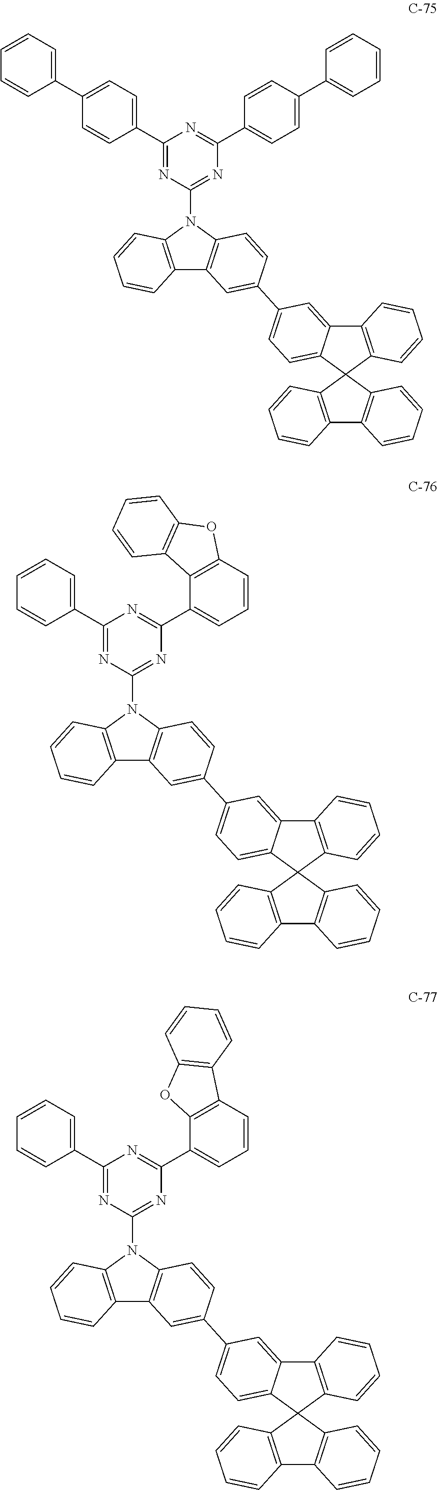 [SMILES: c1ccc(-c2ccc(-c3nc(-c4ccc(-c5ccccc5)cc4)nc(-n4c5ccccc5c5cc(-c6ccc7c(c6)-c6ccccc6C76c7ccccc7-c7ccccc76)ccc54)n3)cc2)cc1 ; c1ccc(-c2nc(-c3cccc4oc5ccccc5c34)nc(-n3c4ccccc4c4cc(-c5ccc6c(c5)-c5ccccc5C65c6ccccc6-c6ccccc65)ccc43)n2)cc1 ; c1ccc(-c2nc(-c3cccc4c3oc3ccccc34)nc(-n3c4ccccc4c4cc(-c5ccc6c(c5)-c5ccccc5C65c6ccccc6-c6ccccc65)ccc43)n2)cc1] 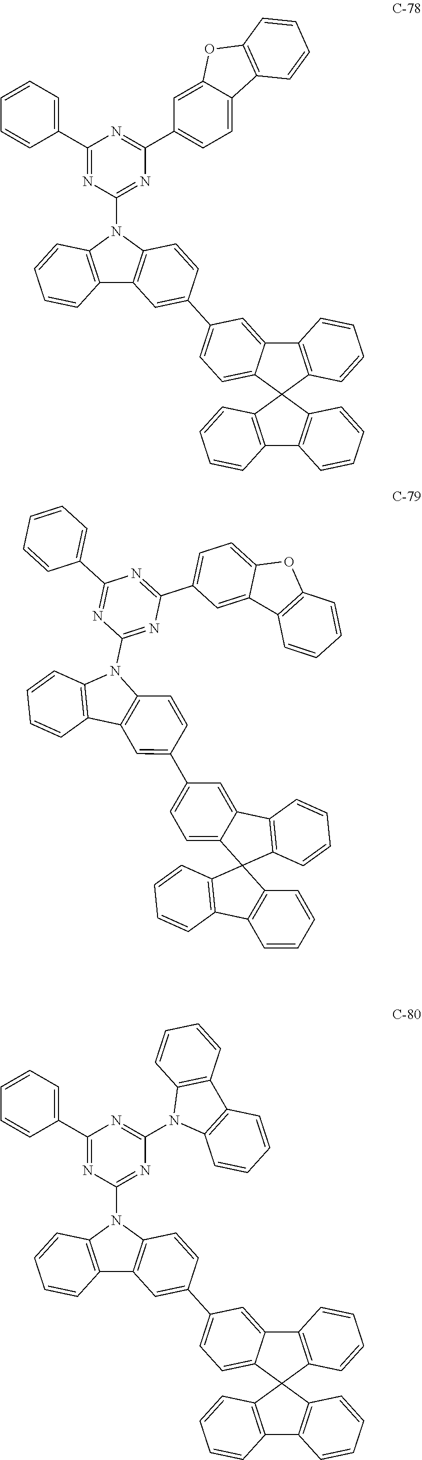 [SMILES: c1ccc(-c2nc(-c3ccc4c(c3)oc3ccccc34)nc(-n3c4ccccc4c4cc(-c5ccc6c(c5)-c5ccccc5C65c6ccccc6-c6ccccc65)ccc43)n2)cc1 ; c1ccc(-c2nc(-c3ccc4oc5ccccc5c4c3)nc(-n3c4ccccc4c4cc(-c5ccc6c(c5)-c5ccccc5C65c6ccccc6-c6ccccc65)ccc43)n2)cc1 ; c1ccc(-c2nc(-n3c4ccccc4c4ccccc43)nc(-n3c4ccccc4c4cc(-c5ccc6c(c5)-c5ccccc5C65c6ccccc6-c6ccccc65)ccc43)n2)cc1] 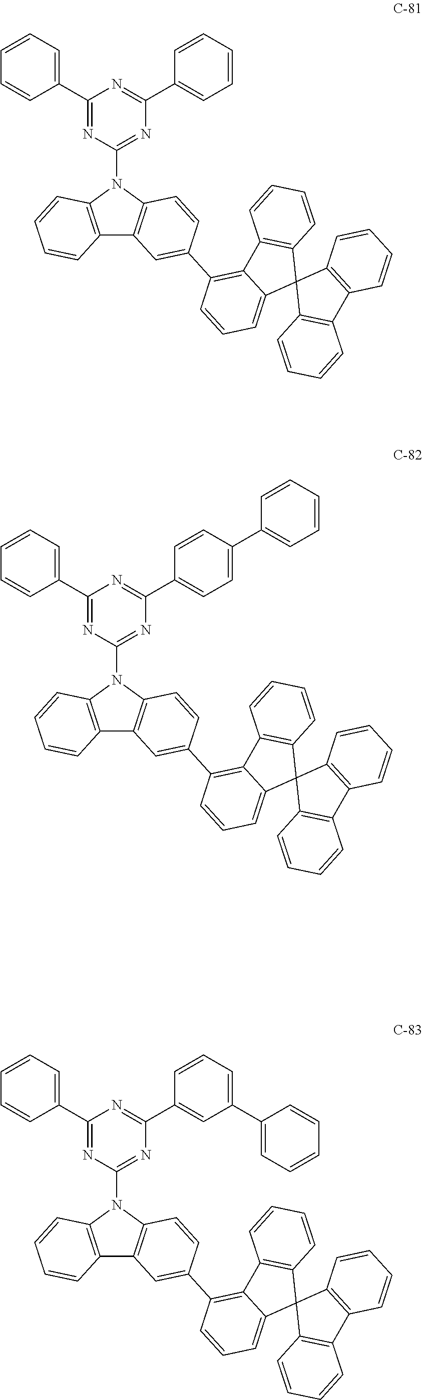 [SMILES: c1ccc(-c2nc(-c3ccccc3)nc(-n3c4ccccc4c4cc(-c5cccc6c5-c5ccccc5C65c6ccccc6-c6ccccc65)ccc43)n2)cc1 ; c1ccc(-c2ccc(-c3nc(-c4ccccc4)nc(-n4c5ccccc5c5cc(-c6cccc7c6-c6ccccc6C76c7ccccc7-c7ccccc76)ccc54)n3)cc2)cc1 ; c1ccc(-c2cccc(-c3nc(-c4ccccc4)nc(-n4c5ccccc5c5cc(-c6cccc7c6-c6ccccc6C76c7ccccc7-c7ccccc76)ccc54)n3)c2)cc1] 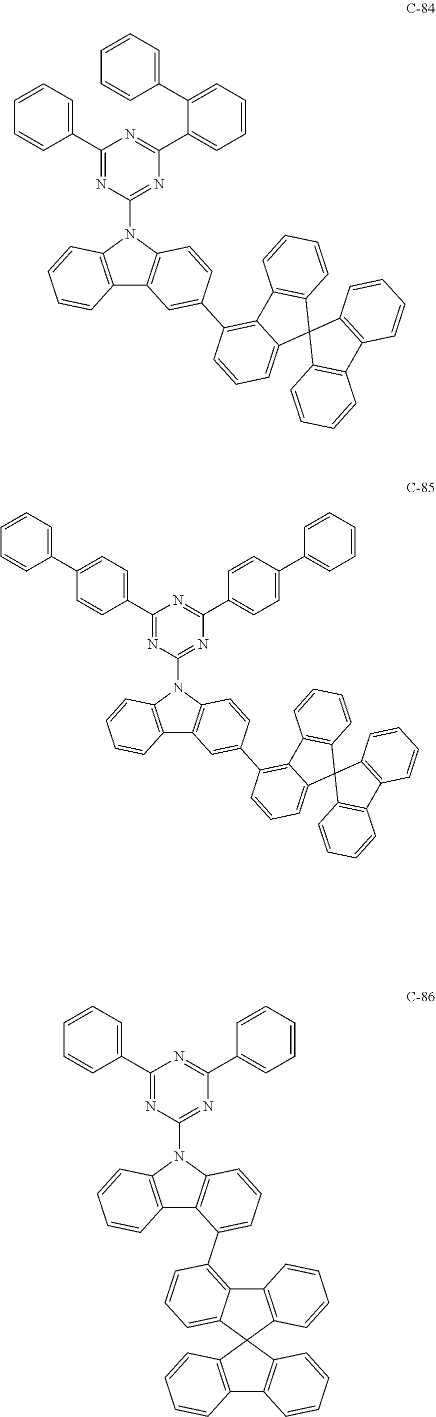 [SMILES: c1ccc(-c2nc(-c3ccccc3-c3ccccc3)nc(-n3c4ccccc4c4cc(-c5cccc6c5-c5ccccc5C65c6ccccc6-c6ccccc65)ccc43)n2)cc1 ; c1ccc(-c2ccc(-c3nc(-c4ccc(-c5ccccc5)cc4)nc(-n4c5ccccc5c5cc(-c6cccc7c6-c6ccccc6C76c7ccccc7-c7ccccc76)ccc54)n3)cc2)cc1 ; c1ccc(-c2nc(-c3ccccc3)nc(-n3c4ccccc4c4c(-c5cccc6c5-c5ccccc5C65c6ccccc6-c6ccccc65)cccc43)n2)cc1] 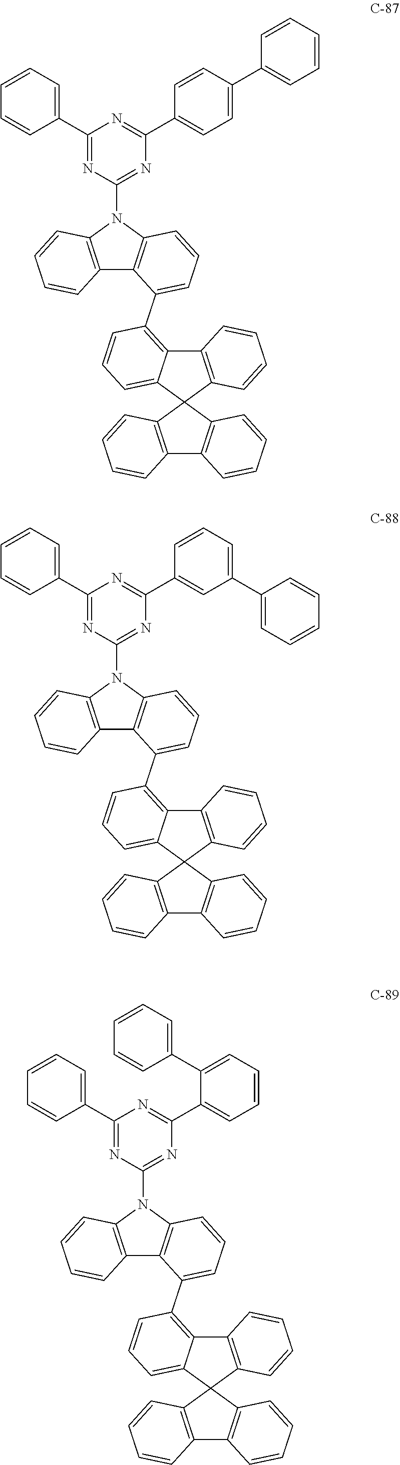 [SMILES: c1ccc(-c2ccc(-c3nc(-c4ccccc4)nc(-n4c5ccccc5c5c(-c6cccc7c6-c6ccccc6C76c7ccccc7-c7ccccc76)cccc54)n3)cc2)cc1 ; c1ccc(-c2cccc(-c3nc(-c4ccccc4)nc(-n4c5ccccc5c5c(-c6cccc7c6-c6ccccc6C76c7ccccc7-c7ccccc76)cccc54)n3)c2)cc1 ; c1ccc(-c2nc(-c3ccccc3-c3ccccc3)nc(-n3c4ccccc4c4c(-c5cccc6c5-c5ccccc5C65c6ccccc6-c6ccccc65)cccc43)n2)cc1] 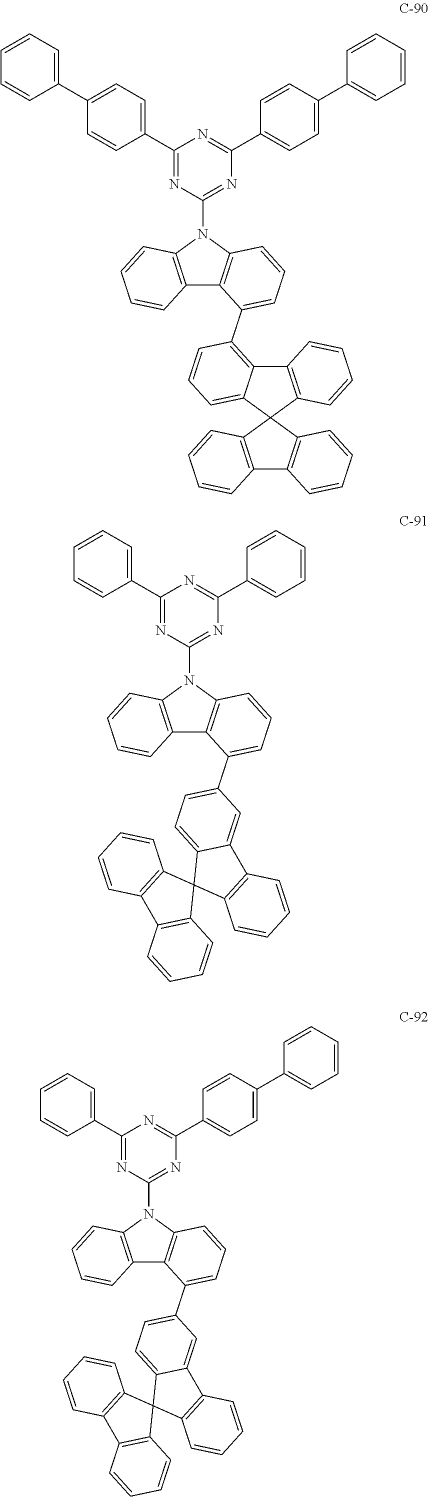 [SMILES: c1ccc(-c2ccc(-c3nc(-c4ccc(-c5ccccc5)cc4)nc(-n4c5ccccc5c5c(-c6cccc7c6-c6ccccc6C76c7ccccc7-c7ccccc76)cccc54)n3)cc2)cc1 ; c1ccc(-c2nc(-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccccc4)nc(-n4c5ccccc5c5c(-c6ccc7c(c6)-c6ccccc6C76c7ccccc7-c7ccccc76)cccc54)n3)cc2)cc1] 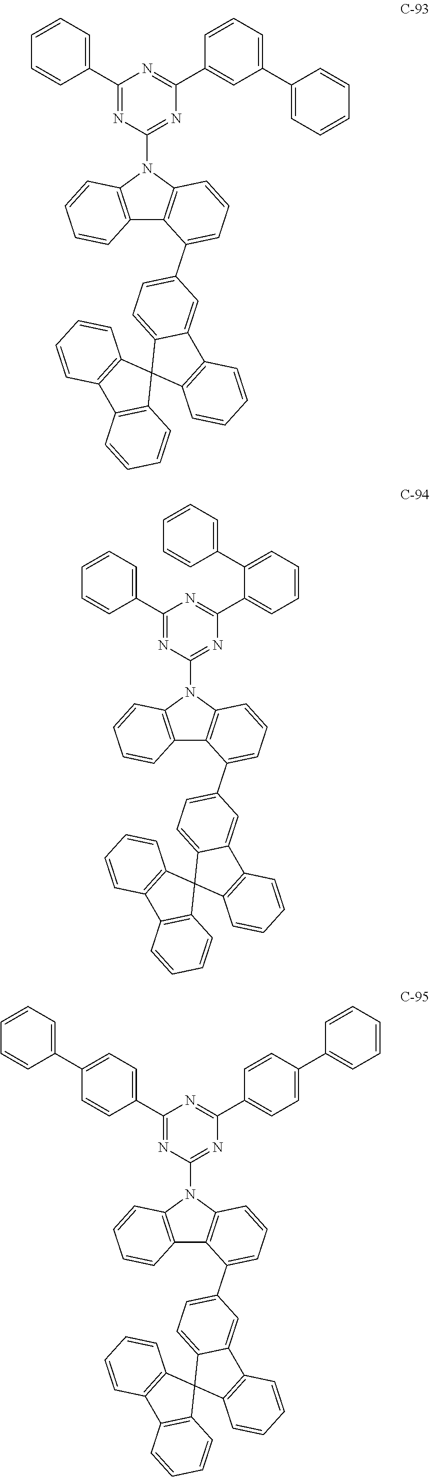 [SMILES: c1ccc(-c2cccc(-c3nc(-c4ccccc4)nc(-n4c5ccccc5c5c(-c6ccc7c(c6)-c6ccccc6C76c7ccccc7-c7ccccc76)cccc54)n3)c2)cc1 ; c1ccc(-c2nc(-c3ccccc3-c3ccccc3)nc(-n3c4ccccc4c4c(-c5ccc6c(c5)-c5ccccc5C65c6ccccc6-c6ccccc65)cccc43)n2)cc1 ; c1ccc(-c2ccc(-c3nc(-c4ccc(-c5ccccc5)cc4)nc(-n4c5ccccc5c5c(-c6ccc7c(c6)-c6ccccc6C76c7ccccc7-c7ccccc76)cccc54)n3)cc2)cc1] 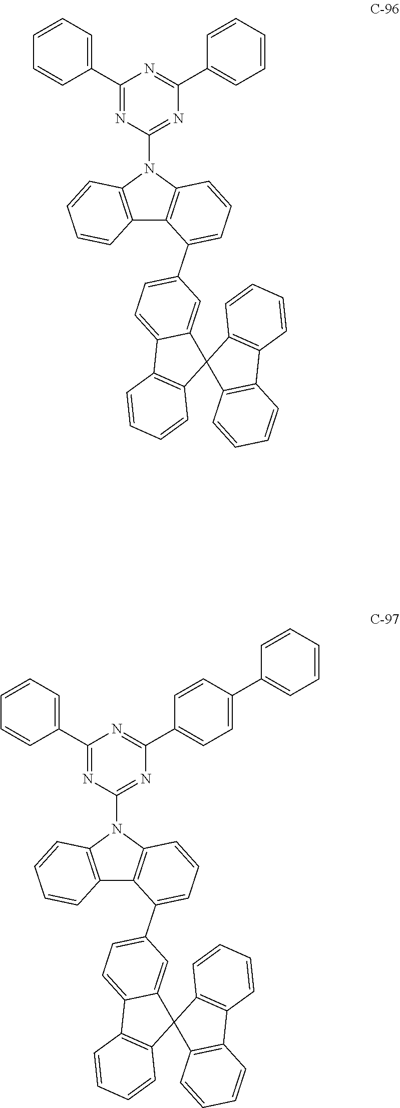 [SMILES: c1ccc(-c2nc(-c3ccccc3)nc(-n3c4ccccc4c4c(-c5ccc6c(c5)C5(c7ccccc7-c7ccccc75)c5ccccc5-6)cccc43)n2)cc1 ; c1ccc(-c2ccc(-c3nc(-c4ccccc4)nc(-n4c5ccccc5c5c(-c6ccc7c(c6)C6(c8ccccc8-c8ccccc86)c6ccccc6-7)cccc54)n3)cc2)cc1] 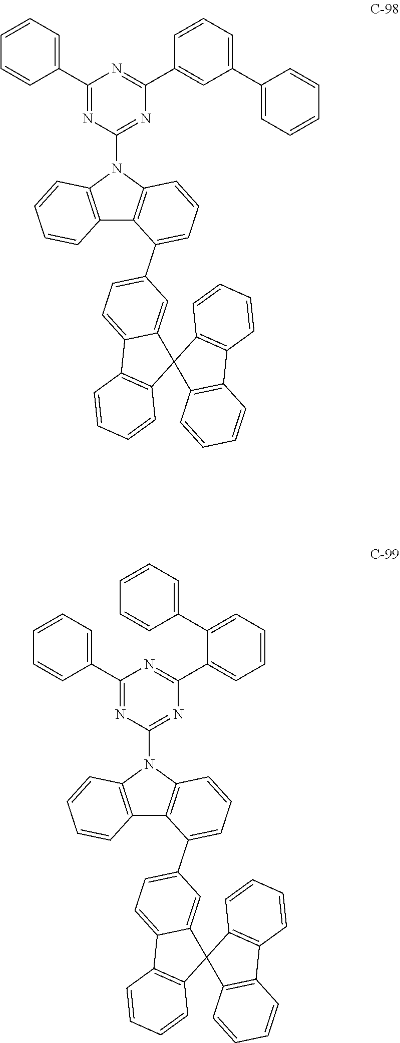 [SMILES: c1ccc(-c2cccc(-c3nc(-c4ccccc4)nc(-n4c5ccccc5c5c(-c6ccc7c(c6)C6(c8ccccc8-c8ccccc86)c6ccccc6-7)cccc54)n3)c2)cc1 ; c1ccc(-c2nc(-c3ccccc3-c3ccccc3)nc(-n3c4ccccc4c4c(-c5ccc6c(c5)C5(c7ccccc7-c7ccccc75)c5ccccc5-6)cccc43)n2)cc1] 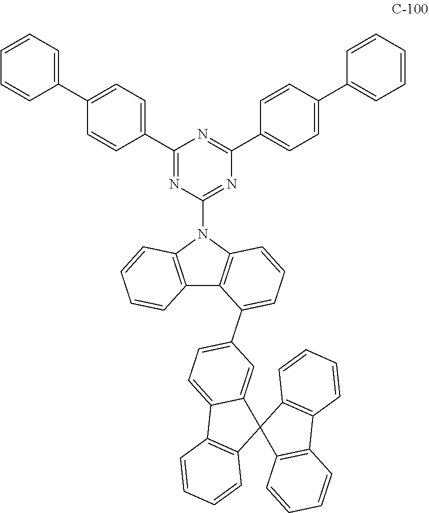 [SMILES: c1ccc(-c2ccc(-c3nc(-c4ccc(-c5ccccc5)cc4)nc(-n4c5ccccc5c5c(-c6ccc7c(c6)C6(c8ccccc8-c8ccccc86)c6ccccc6-7)cccc54)n3)cc2)cc1] 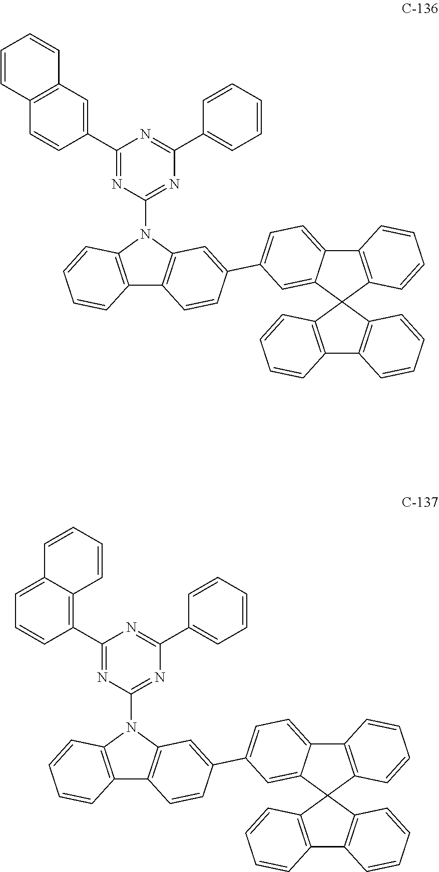 [SMILES: c1ccc(-c2nc(-c3ccc4ccccc4c3)nc(-n3c4ccccc4c4ccc(-c5ccc6c(c5)C5(c7ccccc7-c7ccccc75)c5ccccc5-6)cc43)n2)cc1 ; c1ccc(-c2nc(-c3cccc4ccccc34)nc(-n3c4ccccc4c4ccc(-c5ccc6c(c5)C5(c7ccccc7-c7ccccc75)c5ccccc5-6)cc43)n2)cc1] 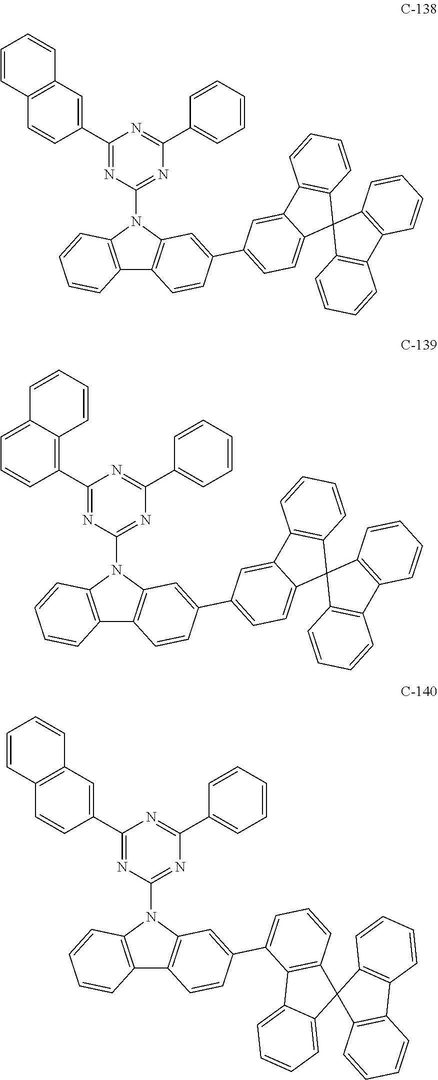 [SMILES: c1ccc(-c2nc(-c3ccc4ccccc4c3)nc(-n3c4ccccc4c4ccc(-c5ccc6c(c5)-c5ccccc5C65c6ccccc6-c6ccccc65)cc43)n2)cc1 ; c1ccc(-c2nc(-c3cccc4ccccc34)nc(-n3c4ccccc4c4ccc(-c5ccc6c(c5)-c5ccccc5C65c6ccccc6-c6ccccc65)cc43)n2)cc1 ; c1ccc(-c2nc(-c3ccc4ccccc4c3)nc(-n3c4ccccc4c4ccc(-c5cccc6c5-c5ccccc5C65c6ccccc6-c6ccccc65)cc43)n2)cc1] 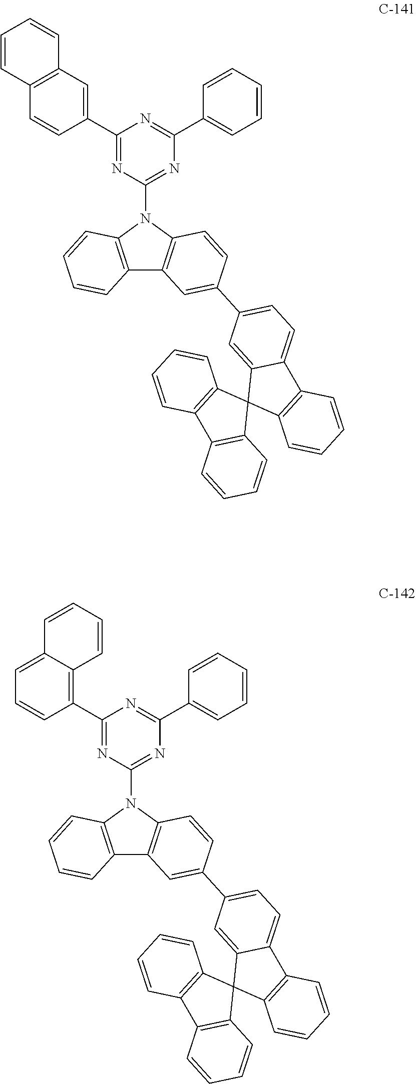 [SMILES: c1ccc(-c2nc(-c3ccc4ccccc4c3)nc(-n3c4ccccc4c4cc(-c5ccc6c(c5)C5(c7ccccc7-c7ccccc75)c5ccccc5-6)ccc43)n2)cc1 ; c1ccc(-c2nc(-c3cccc4ccccc34)nc(-n3c4ccccc4c4cc(-c5ccc6c(c5)C5(c7ccccc7-c7ccccc75)c5ccccc5-6)ccc43)n2)cc1] 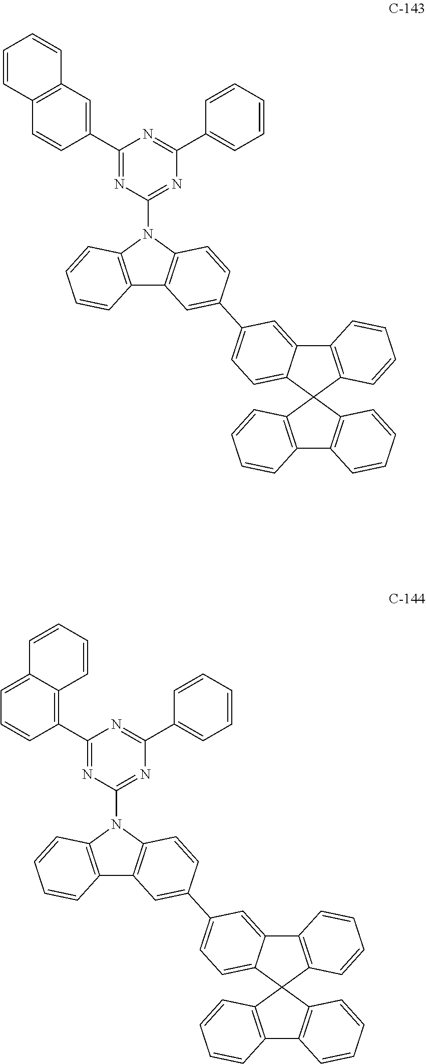 [SMILES: c1ccc(-c2nc(-c3ccc4ccccc4c3)nc(-n3c4ccccc4c4cc(-c5ccc6c(c5)-c5ccccc5C65c6ccccc6-c6ccccc65)ccc43)n2)cc1 ; c1ccc(-c2nc(-c3cccc4ccccc34)nc(-n3c4ccccc4c4cc(-c5ccc6c(c5)-c5ccccc5C65c6ccccc6-c6ccccc65)ccc43)n2)cc1] 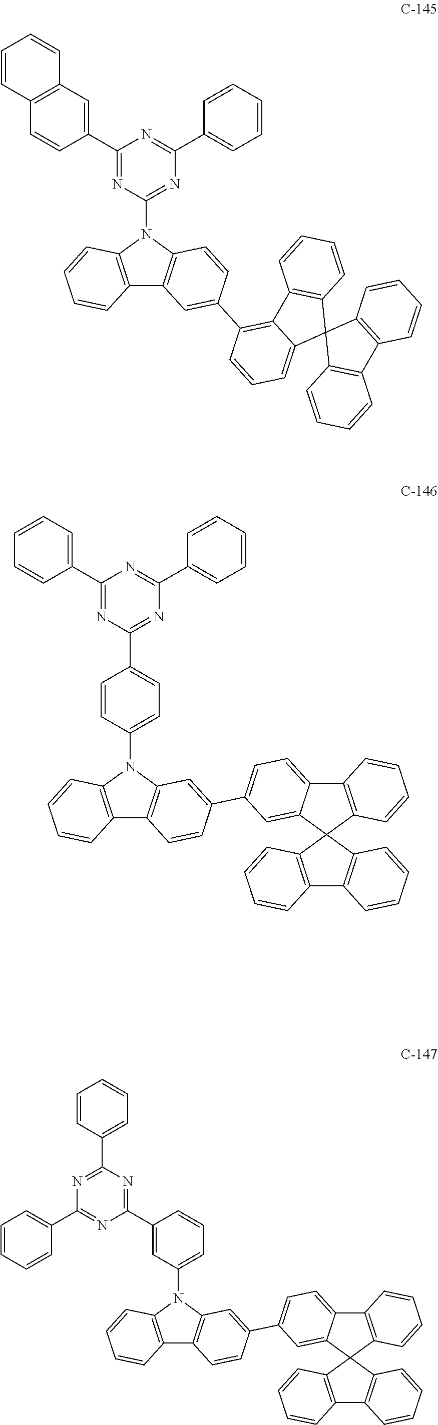 [SMILES: c1ccc(-c2nc(-c3ccc4ccccc4c3)nc(-n3c4ccccc4c4cc(-c5cccc6c5-c5ccccc5C65c6ccccc6-c6ccccc65)ccc43)n2)cc1 ; c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)cc3)n2)cc1 ; c1ccc(-c2nc(-c3ccccc3)nc(-c3cccc(-n4c5ccccc5c5ccc(-c6ccc7c(c6)C6(c8ccccc8-c8ccccc86)c6ccccc6-7)cc54)c3)n2)cc1] 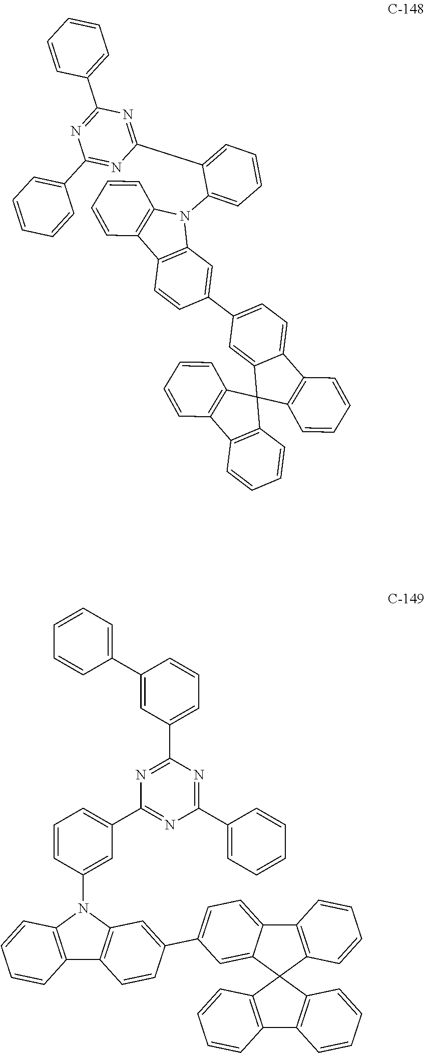 [SMILES: c1ccc(-c2nc(-c3ccccc3)nc(-c3ccccc3-n3c4ccccc4c4ccc(-c5ccc6c(c5)C5(c7ccccc7-c7ccccc75)c5ccccc5-6)cc43)n2)cc1 ; c1ccc(-c2cccc(-c3nc(-c4ccccc4)nc(-c4cccc(-n5c6ccccc6c6ccc(-c7ccc8c(c7)C7(c9ccccc9-c9ccccc97)c7ccccc7-8)cc65)c4)n3)c2)cc1] 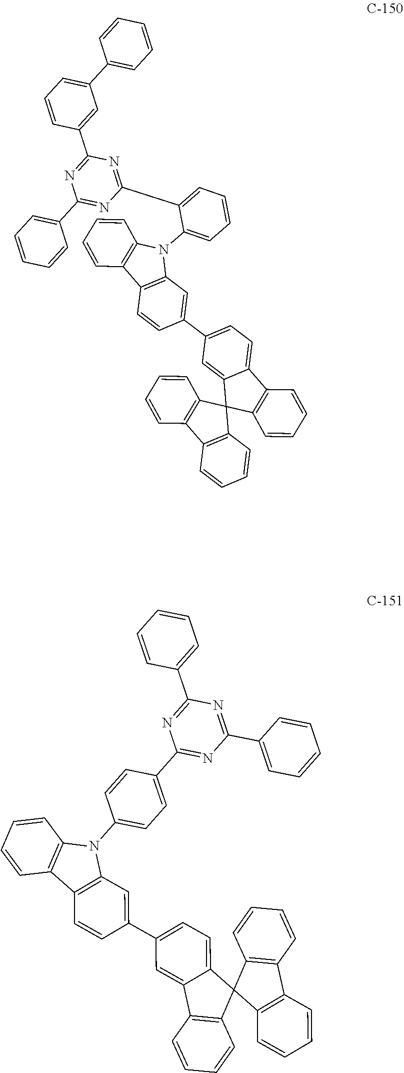 [SMILES: c1ccc(-c2cccc(-c3nc(-c4ccccc4)nc(-c4ccccc4-n4c5ccccc5c5ccc(-c6ccc7c(c6)C6(c8ccccc8-c8ccccc86)c6ccccc6-7)cc54)n3)c2)cc1 ; c1ccc(-c2nc(-c3ccccc3)nc(-c3ccc(-n4c5ccccc5c5ccc(-c6ccc7c(c6)-c6ccccc6C76c7ccccc7-c7ccccc76)cc54)cc3)n2)cc1] 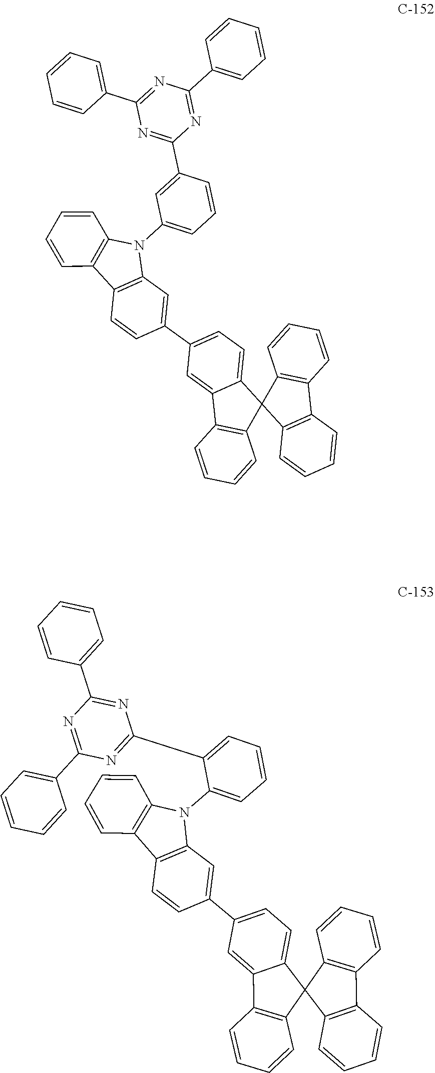 [SMILES: c1ccc(-c2nc(-c3ccccc3)nc(-c3cccc(-n4c5ccccc5c5ccc(-c6ccc7c(c6)-c6ccccc6C76c7ccccc7-c7ccccc76)cc54)c3)n2)cc1 ; c1ccc(-c2nc(-c3ccccc3)nc(-c3ccccc3-n3c4ccccc4c4ccc(-c5ccc6c(c5)-c5ccccc5C65c6ccccc6-c6ccccc65)cc43)n2)cc1] 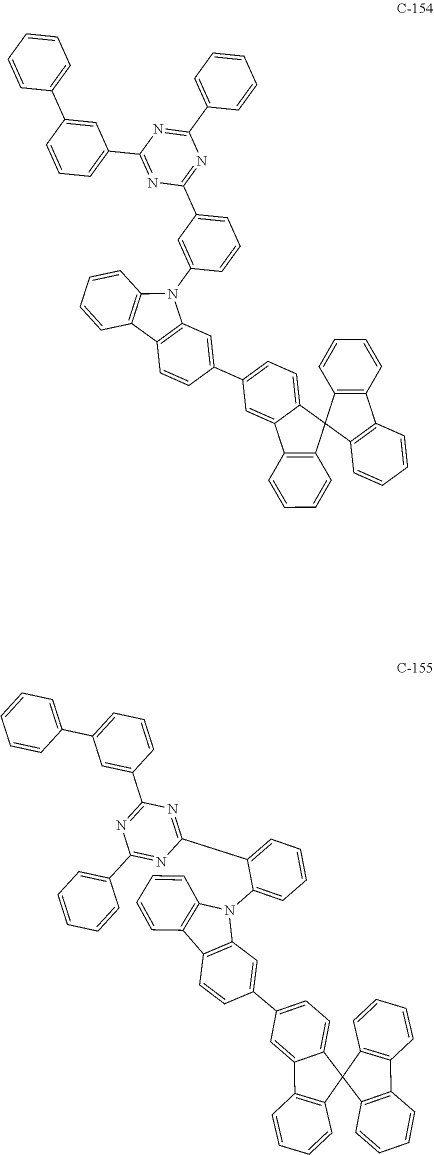 [SMILES: c1ccc(-c2cccc(-c3nc(-c4ccccc4)nc(-c4cccc(-n5c6ccccc6c6ccc(-c7ccc8c(c7)-c7ccccc7C87c8ccccc8-c8ccccc87)cc65)c4)n3)c2)cc1 ; c1ccc(-c2cccc(-c3nc(-c4ccccc4)nc(-c4ccccc4-n4c5ccccc5c5ccc(-c6ccc7c(c6)-c6ccccc6C76c7ccccc7-c7ccccc76)cc54)n3)c2)cc1] 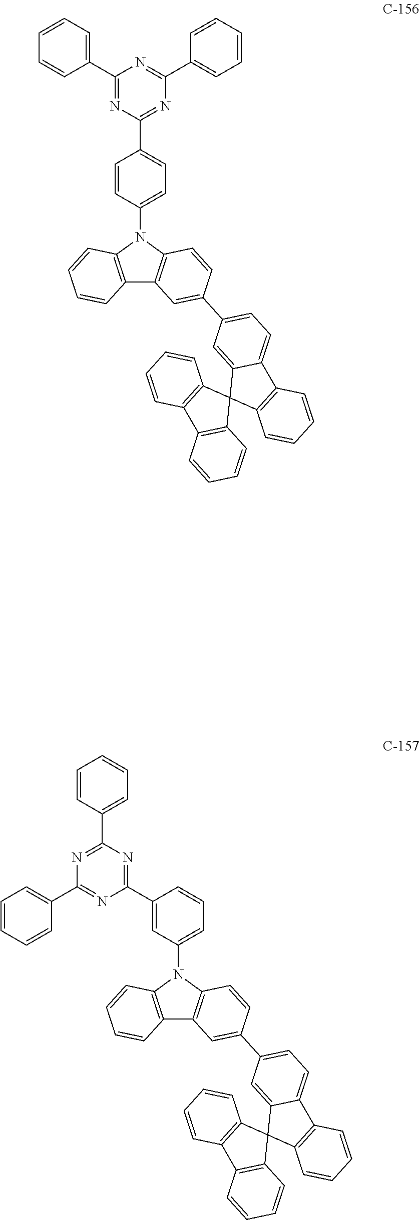 [SMILES: c1ccc(-c2nc(-c3ccccc3)nc(-c3ccc(-n4c5ccccc5c5cc(-c6ccc7c(c6)C6(c8ccccc8-c8ccccc86)c6ccccc6-7)ccc54)cc3)n2)cc1 ; c1ccc(-c2nc(-c3ccccc3)nc(-c3cccc(-n4c5ccccc5c5cc(-c6ccc7c(c6)C6(c8ccccc8-c8ccccc86)c6ccccc6-7)ccc54)c3)n2)cc1] 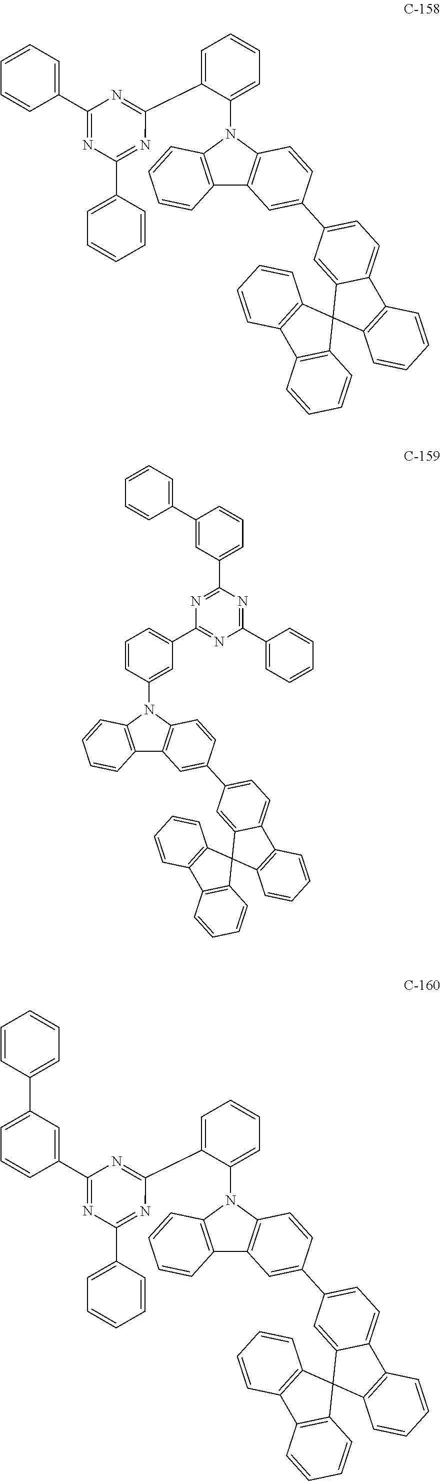 [SMILES: c1ccc(-c2nc(-c3ccccc3)nc(-c3ccccc3-n3c4ccccc4c4cc(-c5ccc6c(c5)C5(c7ccccc7-c7ccccc75)c5ccccc5-6)ccc43)n2)cc1 ; c1ccc(-c2cccc(-c3nc(-c4ccccc4)nc(-c4cccc(-n5c6ccccc6c6cc(-c7ccc8c(c7)C7(c9ccccc9-c9ccccc97)c7ccccc7-8)ccc65)c4)n3)c2)cc1 ; c1ccc(-c2cccc(-c3nc(-c4ccccc4)nc(-c4ccccc4-n4c5ccccc5c5cc(-c6ccc7c(c6)C6(c8ccccc8-c8ccccc86)c6ccccc6-7)ccc54)n3)c2)cc1] 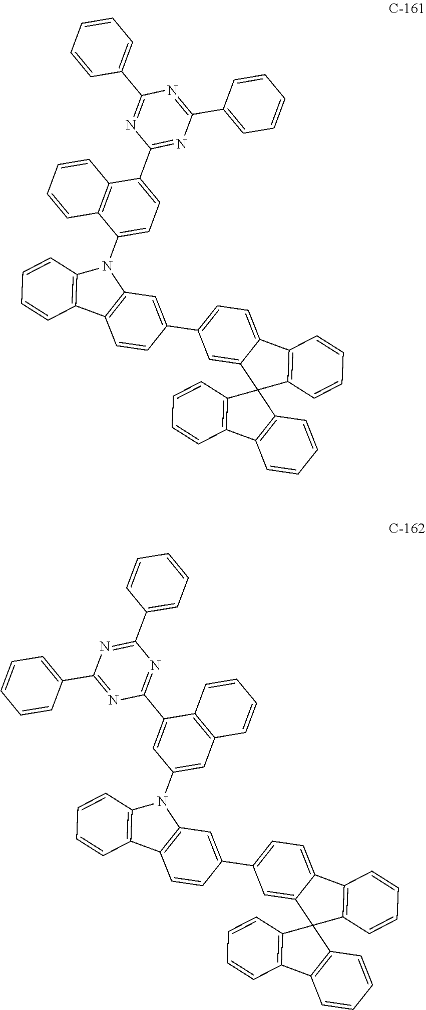 [SMILES: c1ccc(-c2nc(-c3ccccc3)nc(-c3ccc(-n4c5ccccc5c5ccc(-c6ccc7c(c6)C6(c8ccccc8-c8ccccc86)c6ccccc6-7)cc54)c4ccccc34)n2)cc1 ; c1ccc(-c2nc(-c3ccccc3)nc(-c3cc(-n4c5ccccc5c5ccc(-c6ccc7c(c6)C6(c8ccccc8-c8ccccc86)c6ccccc6-7)cc54)cc4ccccc34)n2)cc1] 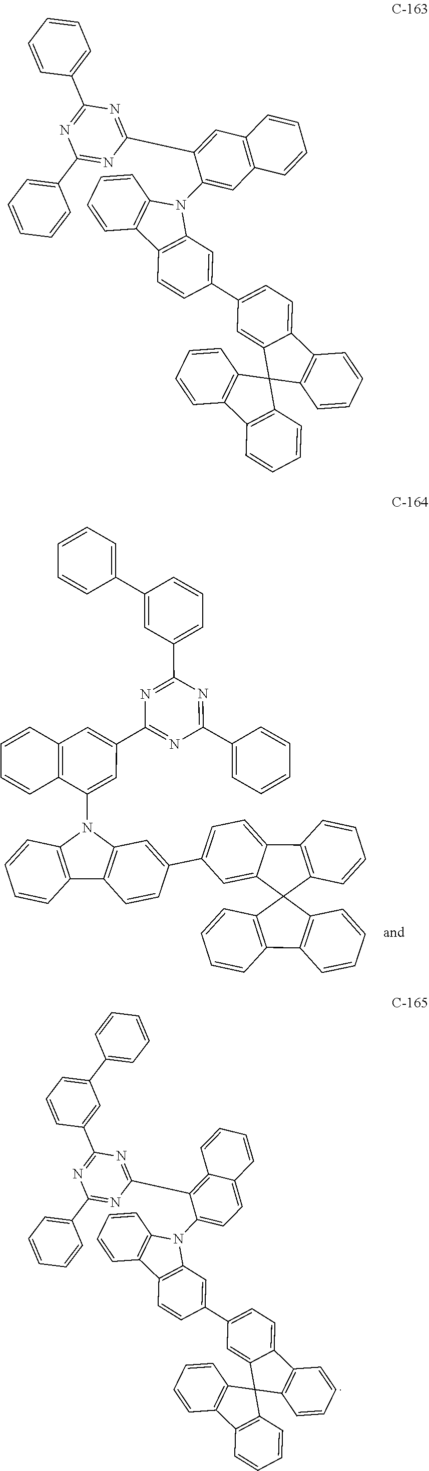 [SMILES: c1ccc(-c2nc(-c3ccccc3)nc(-c3cc4ccccc4cc3-n3c4ccccc4c4ccc(-c5ccc6c(c5)C5(c7ccccc7-c7ccccc75)c5ccccc5-6)cc43)n2)cc1 ; c1ccc(-c2cccc(-c3nc(-c4ccccc4)nc(-c4cc(-n5c6ccccc6c6ccc(-c7ccc8c(c7)C7(c9ccccc9-c9ccccc97)c7ccccc7-8)cc65)c5ccccc5c4)n3)c2)cc1 ; c1ccc(-c2cccc(-c3nc(-c4ccccc4)nc(-c4c(-n5c6ccccc6c6ccc(-c7ccc8c(c7)C7(c9ccccc9-c9ccccc97)c7ccccc7-8)cc65)ccc5ccccc45)n3)c2)cc1]
